(19) United States
(12) Patent Application Publication
(10) Pub. No.: US 2021/0107993 A1
(43) Pub. Date: Apr. 15, 2021

(54) CARTYRIN COMPOSITIONS AND METHODS FOR USE

(71) Applicant: Poseida Therapeutics, Inc., San Diego, CA (US)

(72) Inventors: Eric M. OSTERTAG, San Diego, CA (US); Devon SHEDLOCK, San Diego, CA (US)

(21) Appl. No.: 16/977,547

(22) PCT Filed: Mar. 7, 2019
(86) PCT No.: PCT/US2019/021224

§ 371 Date: Sep. 2, 2020

Related U.S. Application Data
(60) Provisional application No. 62/783,140, filed on Dec. 20, 2018.
(60) Provisional application No. 62/745,151, filed on Oct. 12, 2018.
(60) Provisional application No. 62/639,978, filed on Mar. 7, 2018.

(51) Int. Cl. C07K 16/30; C07K 14/705; C07K 14/725; C12N 5/0783

(52) U.S. Cl. CPC C07K 16/3069; C07K 14/70517; C07K 14/7051; C07K 14/70575; C07K 2319/02; C07K 2318/20; C07K 2319/03; C07K 2319/33; C12N 5/0636

(57) ABSTRACT

Disclosed are Centryin chimeric antigen receptors (CARTyrins), CARTyrin transposons encoding CARTyrins of the disclosure, cells modified to express CARTyrins of the disclosure, as well as methods of making and methods of using the same for adoptive cell therapy. In preferred embodiments, CARTyrins of the disclosure specifically bind to a sequence of prostate specific membrane antigen (PSMA).

### RELATED APPLICATIONS

This application claims the priority benefit of U.S. provisional application No. 62/639,978 filed Mar. 7, 2018, U.S. provisional application No. 62/745,151 filed Oct. 12, 2018 and U.S. provisional application No. 62/783,140 filed Dec. 20, 2018, the contents of each of which are herein incorporated by reference in their entirety.

### INCORPORATION-BY-REFERENCE OF SEQUENCE LISTING

The contents of the file named “POTH-033_001WO_SequenceListing_ST25_R.txt”, which was created on Mar. 7, 2019, and is 6 KB in size are hereby incorporated by reference in their entirety.

### FIELD OF THE DISCLOSURE

The disclosure is directed to molecular biology, and more, specifically, to chimeric antigen receptors, and to transposons containing one or more CARTyrins, as well as methods of making and using the same.

### BACKGROUND

There has been a long-felt but unmet need in the art for a method of directing the specificity of an immune cell without using traditional antibody sequences or fragments thereof. The disclosure provides a superior chimeric antigen receptor.

### SUMMARY

The disclosure provides a chimeric antigen receptor (CAR) comprising: (a) an ectodomain comprising an antigen recognition region, wherein the antigen recognition region comprises at least one Centyrin; (b) a transmembrane domain, and (c) an endodomain comprising at least one costimulatory domain. As used throughout the disclosure, a CAR comprising a Centryin is referred to as a CARTyrin. In certain embodiments, the antigen recognition region may comprise two Centyrins to produce a bi-specific or tandem CARTyrin. In certain embodiments, the antigen recognition region may comprise three Centyrins to produce a tri-specific CARTyrin. In certain embodiments, the ectodomain may further comprise a signal peptide. Alternatively, or in addition, in certain embodiments, the ectodomain may further comprise a hinge between the antigen recognition region and the transmembrane domain. In certain embodiments, the ectodomain may further comprise a signal peptide. Alternatively, or in addition, in certain embodiments, the ectodomain may further comprise a hinge between the antigen recognition region and the transmembrane domain.

The disclosure provides a chimeric antigen receptor (CAR) comprising: (a) an ectodomain comprising an antigen recognition region, wherein the antigen recognition region comprises at least one Centyrin and wherein the at least one Centyrin specifically binds to a sequence of Prostate-Specific Membrane Antigen (PSMA); (b) a transmembrane domain, and (c) an endodomain comprising at least one costimulatory domain. As used throughout the disclosure, a CAR comprising a Centryin is referred to as a CARTyrin. In certain embodiments, the antigen recognition region may comprise two Centyrins to produce a bi-specific or tandem CARTyrin. In certain embodiments, including those wherein the antigen recognition region may comprise two Centyrins to produce a bi-specific or tandem CARTyrin, one or both of the two Centyrins specifically bind(s) to a sequence of PSMA. In some embodiments, a first Centyrin may specifically bind to a first sequence of PSMA and a second Centyrin may specifically bind to a second sequence of PSMA. In some embodiments, the first sequence of PSMA and the second sequence of PSMA are identical. In some embodiments, the first sequence of PSMA and the second sequence of PSMA are not identical. In certain embodiments, the antigen recognition region may comprise three Centyrins to produce a tri-specific CARTyrin. In certain embodiments, including those wherein the antigen recognition region may comprise three Centyrins to produce a tri-specific or tandem CARTyrin, one, two, or three of the three Centyrins specifically bind(s) to a sequence of PSMA. In certain embodiments, a first Centyrin may specifically bind to a first sequence of PSMA, a second Centyrin may specifically bind to a second sequence of PSMA and a third Centyrin may specifically bind to a third sequence of PSMA. In certain embodiments, the ectodomain may further comprise a signal peptide. In certain embodiments, two or more of the first, second or third sequences of PSMA are identical. In certain embodiments, two or more of the first, second or third sequences of PSMA are not identical. In certain embodiments, the first sequence of PSMA, the second sequence of PSMA and the third sequence of PSMA are not identical. Alternatively, or in addition, in certain embodiments, the ectodomain may further comprise a hinge between the antigen recognition region and the transmembrane domain. In certain embodiments, the ectodomain may further comprise a signal peptide. Alternatively, or in addition, in certain embodiments, the ectodomain may further comprise a hinge between the antigen recognition region and the transmembrane domain. As used herein, the term “anti-PSMA CARTyrin” refers to a CARTyrin comprising at least one centyrin that specifically binds a sequence of PSMA.

In certain embodiments of an anti-PSMA CARTyrins of the disclosure, the Centyrin comprises or consists of the amino acid sequence of

(SEQ ID NO: 18000)
MLPAPKNLVVSRVTEDSARLSWDIDEQRDWFESFLIQYQESEKVGEAIVL
TVPGSERSYDLTGLKPGTEYTVSIYGVYHVYRSNPLSAIFTT

(“PSMA5 Centyrin”) or the nucleic acid sequence of

(SEQ ID NO: 18001)
ATGCTGCCTGCACCAAAGAACCTGGTGGTGTCTCGGGTGACCGAGGACTC
TGCCAGACTGAGCTGGGACATCGATGAGCAGAGGGATTGGTTCGAGAGCT
TTCTGATCCAGTATCAGGAGTCCGAGAAAGTGGGCGAGGCCATCGTGCTG
ACAGTGCCTGGCAGCGAGCGGTCCTATGACCTGACCGGCCTGAAGCCAGG
CACAGAGTACACCGTGTCCATCTACGGCGTGTATCACGTGTACAGGTCCA
ATCCTCTGTCTGCCATCTTCACCACA

### (“PSMA5 Centyrin”).

In certain embodiments of an anti-PSMA CARTyrins of the disclosure, the at least one PSMA-specific Centyrin comprises an amino acid sequence having at least 70% identity to the amino acid sequence of

(SEQ ID NO: 18000)
MLPAPKNLVVSRVTEDSARLSWDIDEQRDWFESFLIQYQESEKVGEAIVL
TVPGSERSYDLTGLKPGTEYTVSIYGVYHVYRSNPLSAIFTT.

In certain embodiments, the at least one PSMA-specific Centyrin comprises an amino acid sequence having at least 75%, 80%, 85%, 90%, 95%, 97%, 99%, or any percentage in between of identity to the amino acid sequence of

(SEQ ID NO: 18000)
MLPAPKNLVVSRVTEDSARLSWDIDEQRDWFESFLIQYQESEKVGEAIVL
TVPGSERSYDLTGLKPGTEYTVSIYGVYHVYRSNPLSAIFTT.

In certain embodiments of an anti-PSMA CARTyrins of the disclosure, the Centyrin comprises or consists of the amino acid sequence of

(SEQ ID NO: 18002)
MLPAPKNLVVSRVTEDSARLSWAIDEQRDWFESFLIQYQESEKVGEAIVL
TVPGSERSYDLTGLKPGTEYTVSIYGVYHVYRSNPLSAIFTT

(“PSMA8 Centyrin”) or the nucleic acid sequence of

(SEQ ID NO: 18003)
ATGCTGCCTGCACCAAAGAACCTGGTGGTGTCTCGGGTGACCGAGGACTC
TGCCAGACTGAGCTGGGCCATCGACGAGCAGAGGGATTGGTTCGAGAGCT
TTCTGATCCAGTATCAGGAGTCCGAGAAAGTGGGCGAGGCCATCGTGCTG
ACAGTGCCTGGCAGCGAGCGGTCCTATGATCTGACCGGCCTGAAGCCAGG
CACAGAGTACACCGTGTCCATCTACGGCGTGTATCACGTGTACAGGTCCA
ATCCTCTGTCTGCCATCTTCACCACA

### (“PSMA8 Centyrin”).

In certain embodiments of an anti-PSMA CARTyrins of the disclosure, the at least one PSMA-specific Centyrin comprises an amino acid sequence having at least 70% identity to the amino acid sequence of

(SEQ ID NO: 18002)
MLPAPKNLVVSRVTEDSARLSWAIDEQRDWFESFLIQYQESEKVGEAIVL
TVPGSERSYDLTGLKPGTEYTVSIYGVYHVYRSNPLSAIFTT.

In certain embodiments, the at least one PSMA-specific Centyrin comprises an amino acid sequence having at least 75%, 80%, 85%, 90%, 95%, 97%, 99%, or any percentage in between of identity to the amino acid sequence of

(SEQ ID NO: 18002)
MLPAPKNLVVSRVTEDSARLSWAIDEQRDWFESFLIQYQESEKVGEAIVL
TVPGSERSYDLTGLKPGTEYTVSIYGVYHVYRSNPLSAIFTT.

In certain embodiments of the CARTyrins of the disclosure, the signal peptide may comprise a sequence encoding a human CD2, CD3δ, CD3ε, CD3γ, CD3ζ, CD4, CD8α, CD19, CD28, 4-1BB or GM-CSFR signal peptide. In certain embodiments of the CARTyrins of the disclosure, the signal peptide may comprise a sequence encoding a human CD8α signal peptide. The human CD8α signal peptide may comprise an amino acid sequence comprising MALPVTALLLPLALLLHAARP (SEQ ID NO: 18004). The human CD8α signal peptide may comprise an amino acid sequence comprising MALPVTALLLPLALLLHAARP (SEQ ID NO: 18004) or a sequence having at least 70%, 80%, 90%, 95%, or 99% identity to the an amino acid sequence comprising MALPVTALLLPLALLLHAARP (SEQ ID NO: 18004). The human CD8α signal peptide may be encoded by a nucleic acid sequence comprising

(SEQ ID NO: 18005)
atggcactgccagtcaccgccctgctgctgcctctggctctgctgctgca
cgcagctagacca.

In certain embodiments of the CARTyrins of the disclosure, the transmembrane domain may comprise a sequence encoding a human CD2, CD3δ, CD3ε, CD3γ, CD3ζ, CD4, CD8α, CD19, CD28, 4-1BB or GM-CSFR transmembrane domain. In certain embodiments of the CARTyrins of the disclosure, the transmembrane domain may comprise a sequence encoding a human CD8α transmembrane domain. The CD8α transmembrane domain may comprise an amino acid sequence comprising IYIWAPLAGTCGVLLLSLVITLYC (SEQ ID NO: 18006) or a sequence having at least 70%, 80%, 90%, 95%, or 99% identity to the amino acid sequence comprising IYIWAPLAGTCGVLLLSLVITLYC (SEQ ID NO: 18006). The CD8α transmembrane domain may be encoded by the nucleic acid sequence comprising

(SEQ ID NO: 18007)
atctacatttgggcaccactggccgggacctgtggagtgctgctgctgag
cctggtcatcacactgtactgc.

In certain embodiments of the CARTyrins of the disclosure, the endodomain may comprise a human CD3ζ endodomain.

In certain embodiments of the CARTyrins of the disclosure, the at least one costimulatory domain may comprise a human 4-1BB, CD28, CD40, ICOS, MyD88, OX-40 intracellular segment, or any combination thereof. In certain embodiments of the CARTyrins of the disclosure, the at least one costimulatory domain may comprise a CD28 and/or a 4-1BB costimulatory domain. The CD3ζ costimulatory domain may comprise an amino acid sequence comprising

(SEQ ID NO: 18008)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

or a sequence having at least 70%, 80%, 90%, 95%, or 99% identity to the amino acid sequence comprising

(SEQ ID NO: 18009)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

The CD3ζ costimulatory domain may be encoded by the nucleic acid sequence comprising

(SEQ ID NO: 18010)
cgcgtgaagtttagtcgatcagcagatgccccagcttacaaacagggaca
gaaccagctgtataacgagctgaatctgggccgccgagaggaatatgacg
tgctggataagcggagaggacgcgaccccgaaatgggaggcaagcccagg
cgcaaaaaccctcaggaaggcctgtataacgagctgcagaaggacaaaat
ggcagaagcctattctgagatcggcatgaagggggagcgacggagaggca
aagggcacgatgggctgtaccagggactgagcaccgccacaaaggacacc
tatgatgctctgcatatgcaggcactgcctccaagg.

The 4-1BB costimulatory domain may comprise an amino acid sequence comprising KRGRKKLLYIFKQPFMRPVQTTQEEDGCSCRFPEEEEGGCEL (SEQ ID NO: 18011) or a sequence having at least 70%, 80%, 90%, 95%, or 99% identity to the amino acid sequence comprising

(SEQ ID NO: 18012)
KRGRKKLLYIFKQPFMRPVQTTQEEDGCSCRFPEEEEGGCEL.

The 4-1BB costimulatory domain may be encoded by the nucleic acid sequence comprising

(SEQ ID NO: 18013)
aagagaggcaggaagaaactgctgtatattttcaaacagcccttcatgcg
ccccgtgcagactacccaggaggaagacgggtgctcctgtcgattccctg
aggaagaggaaggcgggtgtgagctg.

The 4-1BB costimulatory domain may be located between the transmembrane domain and the CD28 costimulatory domain.

In certain embodiments of the CARTyrins of the disclosure, the hinge may comprise a sequence derived from a human CD8α, IgG4, and/or CD4 sequence. In certain embodiments of the CARTyrins of the disclosure, the hinge may comprise a sequence derived from a human CD8α sequence. The hinge may comprise a human CD8α amino acid sequence comprising TTTPAPRPPTPAPTIASQPLSLRPEACRPAAGGAVHTRGLDFACD (SEQ ID NO: 18014) or a sequence having at least 70%, 80%, 90%, 95%, or 99% identity to the amino acid sequence comprising

(SEQ ID NO: 18015)
TTTPAPRPPTPAPTIASQPLSLRPEACRPAAGGAVHTRGLDFACD.

The human CD8α hinge amino acid sequence may be encoded by the nucleic acid sequence comprising

(SEQ ID NO: 18016)
ACCACAACCCCTGCCCCCAGACCTCCCACACCCGCCCCTACCATCGCGAG
TCAGCCCCTGAGTCTGAGACCTGAGGCCTGCAGGCCAGCTGCAGGAGGAG
CTGTGCACACCAGGGGCCTGGACTTCGCCTGCGAC
or
(SEQ ID NO: 18017)
ACCACAACCCCTGCCCCCAGACCTCCCACACCCGCCCCTACCATCGCGAG
TCAGCCCCTGAGTCTGAGACCTGAGGCCTGCAGGCCAGCTGCAGGAGGAG
CTGTGCACACCAGGGGCCTGGACTTCGCCTGCGAC.

Centyrins of the disclosure may comprise at least one fibronectin type III (FN3) domain. Centyrins of the disclosure may be capable of specifically binding an antigen. Preferred Centryrins of the disclosure specifically bind a sequence of PSMA. The at least one fibronectin type III (FN3) domain may be derived from a human protein. The human protein may be Tenascin-C. The consensus sequence may comprise

(SEQ ID NO: 18018)
LPAPKNLVVSEVTEDSLRLSWTAPDAAFDSFLIQYQESEKVGEAINLTVP
GSERSYDLTGLKPGTEYTVSIYGVKGGHRSNPLSAEFTT
or
(SEQ ID NO: 18019)
MLPAPKNLVVSEVTEDSLRLSWTAPDAAFDSFLIQYQESEKVGEAINLTV
PGSERSYDLTGLKPGTEYTVSIYGVKGGHRSNPLSAEFTT.

The consensus sequence may be modified at one or more positions within (a) a A-B loop comprising or consisting of the amino acid residues TEDS (SEQ ID NO: 18020) at positions 13-16 of the consensus sequence; (b) a B-C loop comprising or consisting of the amino acid residues TAPDAAF (SEQ ID NO: 18021) at positions 22-28 of the consensus sequence; (c) a C-D loop comprising or consisting of the amino acid residues SEKVGE (SEQ ID NO: 18022) at positions 38-43 of the consensus sequence; (d) a D-E loop comprising or consisting of the amino acid residues GSER (SEQ ID NO: 18023) at positions 51-54 of the consensus sequence; (e) a E-F loop comprising or consisting of the amino acid residues GLKPG (SEQ ID NO: 18024) at positions 60-64 of the consensus sequence; (f) a F-G loop comprising or consisting of the amino acid residues KGGHRSN (SEQ ID NO: 18025) at positions 75-81 of the consensus sequence; or (g) any combination of (a)-(f). Centyrins of the disclosure may comprise a consensus sequence of at least 5 fibronectin type III (FN3) domains, at least 10 fibronectin type III (FN3) domains or at least 15 fibronectin type III (FN3) domains. Centyrins and/or CARTyrins of the disclosure may bind an antigen with at least one affinity selected from a K_{D} of less than or equal to 10⁻⁹ M, less than or equal to 10⁻¹⁰ M, less than or equal to 10⁻¹¹ M, less than or equal to 10⁻¹²M, less than or equal to 10⁻¹³M, less than or equal to 10⁻¹⁴M, and less than or equal to 10⁻¹⁵M. The K_{D} may be determined by surface plasmon resonance.

The disclosure provides a composition comprising a CARTyrin of the disclosure and at least one pharmaceutically acceptable carrier.

The disclosure provides a transposon comprising the CARTyrin of the disclosure.

Transposons of the disclosure may comprise a selection gene for identification, enrichment and/or isolation of cells that express the transposon. Exemplary selection genes encode any gene product (e.g. transcript, protein, enzyme) essential for cell viability and survival. Exemplary selection genes encode any gene product (e.g. transcript, protein, enzyme) essential for conferring resistance to a drug challenge against which the cell is sensitive (or which could be lethal to the cell) in the absence of the gene product encoded by the selection gene. Exemplary selection genes encode any gene product (e.g. transcript, protein, enzyme) essential for viability and/or survival in a cell media lacking one or more nutrients essential for cell viability and/or survival in the absence of the selection gene. Exemplary selection genes include, but are not limited to, neo (conferring resistance to neomycin), TYMS (encoding Thymidylate Synthetase), MGMT (encoding O(6)-methylguanine-DNA methyltransferase), multidrug resistance gene (MDR1), ALDH1 (encoding Aldehyde dehydrogenase 1 family, member A1), FRANCF, RAD51C (encoding RAD51 Paralog C), GCS (encoding glucosylceramide synthase), and NKX2.2 (encoding NK2 Homeobox 2).

Transposons of the disclosure may comprise an inducible proapoptotic polypeptide comprising (a) a ligand binding region, (b) a linker, and (c) a proapoptotic polypeptide, wherein the inducible proapoptotic polypeptide does not comprise a non-human sequence. In certain embodiments, the non-human sequence comprises a restriction site. In certain embodiments, the ligand binding region may be a multimeric ligand binding region. Inducible proapoptotic polypeptides of the disclosure may also be referred to as an “iC9 safety switch”. In certain embodiments, transposons of the disclosure may comprise an inducible caspase polypeptide comprising (a) a ligand binding region, (b) a linker, and (c) a caspase polypeptide, wherein the inducible proapoptotic polypeptide does not comprise a non-human sequence. In certain embodiments, transposons of the disclosure may comprise an inducible caspase polypeptide comprising (a) a ligand binding region, (b) a linker, and (c) a caspase polypeptide, wherein the inducible proapoptotic polypeptide does not comprise a non-human sequence. In certain embodiments, transposons of the disclosure may comprise an inducible caspase polypeptide comprising (a) a ligand binding region, (b) a linker, and (c) a truncated caspase 9 polypeptide, wherein the inducible proapoptotic polypeptide does not comprise a non-human sequence. In certain embodiments of the inducible proapoptotic polypeptides, inducible caspase polypeptides or truncated caspase 9 polypeptides of the disclosure, the ligand binding region may comprise a FK506 binding protein 12 (FKBP12) polypeptide. In certain embodiments, the amino acid sequence of the ligand binding region that comprise a FK506 binding protein 12 (FKBP12) polypeptide may comprise a modification at position 36 of the sequence. The modification may be a substitution of valine (V) for phenylalanine (F) at position 36 (F36V). In certain embodiments, the FKBP12 polypeptide is encoded by an amino acid sequence comprising

(SEQ ID NO: 18026)
GVQVETISPGDGRTFPKRGQTCVVHYTGMLEDGKKVDSSRDRNKPFKFML
GKQEVIRGWEEGVAQMSVGQRAKLTISPDYAYGATGHPGIIPPHATLVFD
VELLKLE.

In certain embodiments, the FKBP12 polypeptide is encoded by a nucleic acid sequence comprising

(SEQ ID NO: 18027)
GGGGTCCAGGTCGAGACTATTTCACCAGGGGATGGGCGAACATTTCCAAA
AAGGGGCCAGACTTGCGTCGTGCATTACACCGGGATGCTGGAGGACGGGA
AGAAAGTGGACAGCTCCAGGGATCGCAACAAGCCCTTCAAGTTCATGCTG
GGAAAGCAGGAAGTGATCCGAGGATGGGAGGAAGGCGTGGCACAGATGTC
AGTCGGCCAGCGGGCCAAACTGACCATTAGCCCTGACTACGCTTATGGAG
CAACAGGCCACCCAGGGATCATTCCCCCTCATGCCACCCTGGTCTTCGAT
GTGGAACTGCTGAAGCTGGAG.

In certain embodiments, the induction agent specific for the ligand binding region may comprise a FK506 binding protein 12 (FKBP12) polypeptide having a substitution of valine (V) for phenylalanine (F) at position 36 (F36V) comprises AP20187 and/or AP1903, both synthetic drugs.

In certain embodiments of the inducible proapoptotic polypeptides, inducible caspase polypeptides or truncated caspase 9 polypeptides of the disclosure, the linker region is encoded by an amino acid comprising GGGGS (SEQ ID NO: 18028) or a nucleic acid sequence comprising GGAGGAGGAGGATCC (SEQ ID NO: 18029). In certain embodiments, the nucleic acid sequence encoding the linker does not comprise a restriction site.

In certain embodiments of the truncated caspase 9 polypeptides of the disclosure, the truncated caspase 9 polypeptide is encoded by an amino acid sequence that does not comprise an arginine (R) at position 87 of the sequence. Alternatively, or in addition, in certain embodiments of the inducible proapoptotic polypeptides, inducible caspase polypeptides or truncated caspase 9 polypeptides of the disclosure, the truncated caspase 9 polypeptide is encoded by an amino acid sequence that does not comprise an alanine (A) at position 282 the sequence. In certain embodiments of the inducible proapoptotic polypeptides, inducible caspase polypeptides or truncated caspase 9 polypeptides of the disclosure, the truncated caspase 9 polypeptide is encoded by an amino acid comprising

(SEQ ID NO: 18030)
GFGDVGALESLRGNADLAYILSMEPCGHCLIINNVNFCRESGLRTRTGSN
IDCEKLRRRFSSLHFMVEVKGDLTAKKMVLALLELAQQDHGALDCCVVVI
LSHGCQASHLQFPGAVYGTDGCPVSVEKIVNIFNGTSCPSLGGKPKLFFI
QACGGEQKDHGFEVASTSPEDESPGSNPEPDATPFQEGLRTFDQLDAISS
LPTPSDIFVSYSTFPGFVSWRDPKSGSWYVETLDDIFEQWAHSEDLQSLL
LRVANAVSVKGIYKQMPGCFNFLRKKLFFKTS

or a nucleic acid sequence comprising

(SEQ ID NO: 18031)
GGATTTGGGGACGTGGGGGCCCTGGAGTCTCTGCGAGGAAATGCCGATCT
GGCTTACATCCTGAGCATGGAACCCTGCGGCCACTGTCTGATCATTAACA
ATGTGAACTTCTGCAGAGAAAGCGGACTGCGAACACGGACTGGCTCCAAT
ATTGACTGTGAGAAGCTGCGGAGAAGGTTCTCTAGTCTGCACTTTATGGT
CGAAGTGAAAGGGGATCTGACCGCCAAGAAAATGGTGCTGGCCCTGCTGG
AGCTGGCTCAGCAGGACCATGGAGCTCTGGATTGCTGCGTGGTCGTGATC
CTGTCCCACGGGTGCCAGGCTTCTCATCTGCAGTTCCCCGGAGCAGTGTA
CGGAACAGACGGCTGTCCTGTCAGCGTGGAGAAGATCGTCAACATCTTCA
ACGGCACTTCTTGCCCTAGTCTGGGGGGAAAGCCAAAACTGTTCTTTATC
CAGGCCTGTGGCGGGGAACAGAAAGATCACGGCTTCGAGGTGGCCAGCAC
CAGCCCTGAGGACGAATCACCAGGGAGCAACCCTGAACCAGATGCAACTC
CATTCCAGGAGGGACTGAGGACCTTTGACCAGCTGGATGCTATCTCAAGC
CTGCCCACTCCTAGTGACATTTTCGTGTCTTACAGTACCTTCCCAGGCTT
TGTCTCATGGCGCGATCCCAAGTCAGGGAGCTGGTACGTGGAGACACTGG
ACGACATCTTTGAACAGTGGGCCCATTCAGAGGACCTGCAGAGCCTGCTG
CTGCGAGTGGCAAACGCTGTCTCTGTGAAGGGCATCTACAAACAGATGCC
CGGGTGCTTCAATTTTCTGAGAAAGAAACTGTTCTTTAAGACTTCC.

In certain embodiments of the inducible proapoptotic polypeptides, wherein the polypeptide comprises a truncated caspase 9 polypeptide, the inducible proapoptotic polypeptide is encoded by an amino acid sequence comprising

(SEQ ID NO: 18032)
GVQVETISPGDGRTFPKRGQTCVVHYTGMLEDGKKVDSSRDRNKPFKFML
GKQEVIRGWEEGVAQMSVGQRAKLTISPDYAYGATGHPGIIPPHATLVFD
VELLKLEGGGGSGFGDVGALESLRGNADLAYILSMEPCGHCLIINNVNFC
RESGLRTRTGSNIDCEKLRRRFSSLHFMVEVKGDLTAKKMVLALLELAQQ
DHGALDCCVVVILSHGCQASHLQFPGAVYGTDGCPVSVEKIVNIFNGTSC
PSLGGKPKLFFIQACGGEQKDHGFEVASTSPEDESPGSNPEPDATPFQEG
LRTFDQLDAISSLPTPSDIFVSYSTFPGFVSWRDPKSGSWYVETLDDIFE
QWAHSEDLQSLLLRVANAVSVKGIYKQMPGCFNFLRKKLFFKTS

or the nucleic acid sequence comprising

(SEQ ID NO: 18033)
ggggtccaggtcgagactatttcaccaggggatgggcgaacatttccaaa
aaggggccagacttgcgtcgtgcattacaccgggatgctggaggacggga
agaaagtggacagctccagggatcgcaacaagcccttcaagttcatgctg
ggaaagcaggaagtgatccgaggatgggaggaaggcgtggcacagatgtc
agtcggccagcgggccaaactgaccattagccctgactacgcttatggag
caacaggccacccagggatcattccccctcatgccaccctggtcttcgat
gtggaactgctgaagctggagggaggaggaggatccggatttggggacgt
gggggccctggagtctctgcgaggaaatgccgatctggcttacatcctga
gcatggaaccctgcggccactgtctgatcattaacaatgtgaacttctgc
agagaaagcggactgcgaacacggactggctccaatattgactgtgagaa
gctgcggagaaggttctctagtctgcactttatggtcgaagtgaaagggg
atctgaccgccaagaaaatggtgctggccctgctggagctggctcagcag
gaccatggagctctggattgctgcgtggtcgtgatcctgtcccacgggtg
ccaggcttctcatctgcagttccccggagcagtgtacggaacagacggct
gtcctgtcagcgtggagaagatcgtcaacatcttcaacggcacttcttgc
cctagtctggggggaaagccaaaactgttctttatccaggcctgtggcgg
ggaacagaaagatcacggcttcgaggtggccagcaccagccctgaggacg
aatcaccagggagcaaccctgaaccagatgcaactccattccaggaggga
ctgaggacctttgaccagctggatgctatctcaagcctgcccactcctag
tgacattacgtgtcttacagtaccttcccaggctttgtctcatggcgcga
tcccaagtcagggagctggtacgtggagacactggacgacatctttgaac
agtgggcccattcagaggacctgcagagcctgctgctgcgagtggcaaac
gctgtctctgtgaagggcatctacaaacagatgcccgggtgcttcaattt
tctgagaaagaaactgttattaagacttcc.

Transposons of the disclosure may comprise at least one self-cleaving peptide(s) located, for example, between one or more Centyrin(s) or CARTyrin(s) of the disclosure and a selection gene of the disclosure. Transposons of the disclosure may comprise at least one self-cleaving peptide(s) located, for example, between one or more Centyrin(s) or CARTyrin(s) of the disclosure and an inducible proapoptotic polypeptide of the disclosure. Transposons of the disclosure may comprise at least two self-cleaving peptide(s), a first self-cleaving peptide located, for example, upstream or immediately upstream of an inducible proapoptotic polypeptide of the disclosure and a second first self-cleaving peptide located, for example, downstream or immediately upstream of an inducible proapoptotic polypeptide of the disclosure.

The at least one self-cleaving peptide may comprise, for example, a T2A peptide, GSG-T2A peptide, an E2A peptide, a GSG-E2A peptide, an F2A peptide, a GSG-F2A peptide, a P2A peptide, or a GSG-P2A peptide. A T2A peptide may comprise an amino acid sequence comprising EGRGSLLTCGDVEENPGP (SEQ ID NO: 18034) or a sequence having at least 70%, 80%, 90%, 95%, or 99% identity to the amino acid sequence comprising EGRGSLLTCGDVEENPGP (SEQ ID NO: 18035). A GSG-T2A peptide may comprise an amino acid sequence comprising GSGEGRGSLLTCGDVEENPGP (SEQ ID NO: 18036) or a sequence having at least 70%, 80%, 90%, 95%, or 99% identity to the amino acid sequence comprising GSGEGRGSLLTCGDVEENPGP (SEQ ID NO: 18037). A GSG-T2A peptide may comprise a nucleic acid sequence comprising

(SEQ ID NO: 18038)
ggatctggagagggaaggggaagcctgctgacctgtggagacgtggagga
aaacccaggacca.

An E2A peptide may comprise an amino acid sequence comprising QCTNYALLKLAGDVESNPGP (SEQ ID NO: 18039) or a sequence having at least 70%, 80%, 90%, 95%, or 99% identity to the amino acid sequence comprising QCTNYALLKLAGDVESNPGP (SEQ ID NO: 18040). A GSG-E2A peptide may comprise an amino acid sequence comprising GSGQCTNYALLKLAGDVESNPGP (SEQ ID NO: 18041) or a sequence having at least 70%, 80%, 90%, 95%, or 99% identity to the amino acid sequence comprising GSGQCTNYALLKLAGDVESNPGP (SEQ ID NO: 18042). An F2A peptide may comprise an amino acid sequence comprising VKQTLNFDLLKLAGDVESNPGP (SEQ ID NO: 18043) or a sequence having at least 70%, 80%, 90%, 95%, or 99% identity to the amino acid sequence comprising VKQTLNFDLLKLAGDVESNPGP (SEQ ID NO: 18044). A GSG-F2A peptide may comprise an amino acid sequence comprising GSGVKQTLNFDLLKLAGDVESNPGP (SEQ ID NO: 18045) or a sequence having at least 70%, 80%, 90%, 95%, or 99% identity to the amino acid sequence comprising GSGVKQTLNFDLLKLAGDVESNPGP (SEQ ID NO: 18046). A P2A peptide may comprise an amino acid sequence comprising ATNFSLLKQAGDVEENPGP (SEQ ID NO: 18047) or a sequence having at least 70%, 80%, 90%, 95%, or 99% identity to the amino acid sequence comprising ATNFSLLKQAGDVEENPGP (SEQ ID NO: 18048). A GSG-P2A peptide may comprise an amino acid sequence comprising GSGATNFSLLKQAGDVEENPGP (SEQ ID NO: 18049) or a sequence having at least 70%, 80%, 90%, 95%, or 99% identity to the amino acid sequence comprising GSGATNFSLLKQAGDVEENPGP (SEQ ID NO: 18050).

Transposons of the disclosure may comprise a first and a second self-cleaving peptide, the first self-cleaving peptide located, for example, upstream of one or more Centyrin(s) or CARTyrin(s) of the disclosure the second self-cleaving peptide located, for example, downstream of the one or more Centyrin(s) or CARTyrin(s) of the disclosure. The first and/or the second self-cleaving peptide may comprise, for example, a T2A peptide, GSG-T2A peptide, an E2A peptide, a GSG-E2A peptide, an F2A peptide, a GSG-F2A peptide, a P2A peptide, or a GSG-P2A peptide. A T2A peptide may comprise an amino acid sequence comprising EGRGSLLTCGDVEENPGP (SEQ ID NO: 18034) or a sequence having at least 70%, 80%, 90%, 95%, or 99% identity to the amino acid sequence comprising EGRGSLLTCGDVEENPGP (SEQ ID NO: 18035). A GSG-T2A peptide may comprise an amino acid sequence comprising GSGEGRGSLLTCGDVEENPGP (SEQ ID NO: 18036) or a sequence having at least 70%, 80%, 90%, 95%, or 99% identity to the amino acid sequence comprising GSGEGRGSLLTCGDVEENPGP (SEQ ID NO: 18037). A GSG-T2A peptide may comprise a nucleic acid sequence comprising

(SEQ ID NO: 18038)
ggatctggagagggaaggggaagcctgctgacctgtggagacgtggagga
aaacccaggacca.

An E2A peptide may comprise an amino acid sequence comprising QCTNYALLKLAGDVESNPGP (SEQ ID NO: 18039) or a sequence having at least 70%, 80%, 90%, 95%, or 99% identity to the amino acid sequence comprising QCTNYALLKLAGDVESNPGP (SEQ ID NO: 18040). A GSG-E2A peptide may comprise an amino acid sequence comprising GSGQCTNYALLKLAGDVESNPGP (SEQ ID NO: 18041) or a sequence having at least 70%, 80%, 90%, 95%, or 99% identity to the amino acid sequence comprising GSGQCTNYALLKLAGDVESNPGP (SEQ ID NO: 18042). An F2A peptide may comprise an amino acid sequence comprising VKQTLNFDLLKLAGDVESNPGP (SEQ ID NO: 18043) or a sequence having at least 70%, 80%, 90%, 95%, or 99% identity to the amino acid sequence comprising VKQTLNFDLLKLAGDVESNPGP (SEQ ID NO: 18044). A GSG-F2A peptide may comprise an amino acid sequence comprising GSGVKQTLNFDLLKLAGDVESNPGP (SEQ ID NO: 18045) or a sequence having at least 70%, 80%, 90%, 95%, or 99% identity to the amino acid sequence comprising GSGVKQTLNFDLLKLAGDVESNPGP (SEQ ID NO: 18046). A P2A peptide may comprise an amino acid sequence comprising ATNFSLLKQAGDVEENPGP (SEQ ID NO: 18047) or a sequence having at least 70%, 80%, 90%, 95%, or 99% identity to the amino acid sequence comprising ATNFSLLKQAGDVEENPGP (SEQ ID NO: 18048). A GSG-P2A peptide may comprise an amino acid sequence comprising GSGATNFSLLKQAGDVEENPGP (SEQ ID NO: 18049) or a sequence having at least 70%, 80%, 90%, 95%, or 99% identity to the amino acid sequence comprising GSGATNFSLLKQAGDVEENPGP (SEQ ID NO: 18050).

In some embodiments of the transposons of the disclosure, including those comprising a CAR of the disclosure, the transposon further comprises a sequence encoding a chimeric stimulatory receptor (CSR). In some embodiments, the CSR comprises: (a) an ectodomain comprising an activation component; (b) a transmembrane domain; and (c) an endodomain comprising at least one signal transduction domain; wherein the combination of (a), (b) and (c) is non-naturally occurring. In some embodiments, the activation component of (a) is isolated or derived from a first protein. In some embodiments, the at least one signal transduction domain of (c) is isolated or derived from a second protein. In some embodiments, the first protein and the second protein are not identical. In some embodiments, the Activation component comprises one or more of a component of a human transmembrane receptor, a human cell-surface receptor, a T-cell Receptor (TCR), a component of a TCR complex, a component of a TCR co-receptor, a component of a TCR co-stimulatory protein, a component of a TCR inhibitory protein, a cytokine receptor, and a chemokine receptor. In some embodiments, the activation component comprises a portion of one or more of a component of a T-cell Receptor (TCR), a component of a TCR complex, a component of a TCR co-receptor, a component of a TCR co-stimulatory protein, a component of a TCR inhibitory protein, a cytokine receptor, and a chemokine receptor to which an agonist of the Activation component binds. In some embodiments, the activation component comprises a CD2 protein or a portion thereof to which an agonist binds. In some embodiments, the signal transduction domain comprises one or more of a component of a human signal transduction domain, T-cell Receptor (TCR), a component of a TCR complex, a component of a TCR co-receptor, a component of a TCR co-stimulatory protein, a component of a TCR inhibitory protein, a cytokine receptor, and a chemokine receptor. In some embodiments, the signal transduction domain comprises a CD3 protein. In some embodiments, the CD3 protein comprises a CD3ζ protein. In some embodiments, the endodomain further comprises a cytoplasmic domain. In some embodiments, the cytoplasmic domain is isolated or derived from a third protein. In some embodiments, the first protein and the third protein are identical. In some embodiments, the ectodomain further comprises a signal peptide. In some embodiments, the signal peptide is derived from a fourth protein. In some embodiments, the first protein and the fourth protein are identical. In some embodiments, the transmembrane domain is isolated or derived from a fifth protein. In some embodiments, the first protein and the fifth protein are identical. In some embodiments, the Activation component does not bind a naturally-occurring molecule. In some embodiments, the CSR does not transduce a signal upon binding of the activation component to a naturally-occurring molecule. In some embodiments, the ectodomain comprises a modification. In some embodiments, the modification comprises a mutation or a truncation of a sequence encoding the activation component when compared to a wild type sequence of the first protein. In some embodiments, the Activation component binds to a non-naturally occurring molecule. In some embodiments, the CSR selectively transduces a signal upon binding of the Activation component to a non-naturally occurring molecule.

In some embodiments of the transposons of the disclosure, the transposon is a piggyBac or a piggyBac-like transposon.

In some embodiments of the transposons of the disclosure, the transposon is a TcBuster transposon.

In some embodiments of the transposons of the disclosure, the transposon is a Sleeping Beauty transposon.

In some embodiments of the transposons of the disclosure, the transposon is a Helraiser transposon.

In some embodiments of the transposons of the disclosure, the transposon is a Tol2 transposon.

The disclosure provides a composition comprising the transposon the disclosure. In certain embodiments, the composition may further comprise a plasmid comprising a sequence encoding a transposase enzyme. The sequence encoding a transposase enzyme may be an mRNA sequence.

The disclosure provides a composition comprising a CAR of the disclosure. In some embodiments, the composition further comprises a CSR of the disclosure or a sequence encoding the CSR. In some embodiments, the sequence encoding the CSR comprises DNA. In some embodiments, the sequence encoding the CSR comprises RNA. In some embodiments, the sequence encoding the CSR comprises messenger RNA (mRNA). In some embodiments, upon introduction to a cell of the disclosure, the CSR or the sequence encoding the CSR is stably integrated by the cell. In some embodiments, upon introduction to a cell of the disclosure, the CSR or the sequence encoding the CSR is not stably integrated by the cell. In some embodiments, upon introduction to a cell of the disclosure, the CSR or the sequence encoding the CSR is stably expressed by the cell. In some embodiments, upon introduction to a cell of the disclosure, the CSR or the sequence encoding the CSR is transiently expressed by the cell. In some embodiments, upon introduction to a cell of the disclosure, the CSR or the sequence encoding the CSR comprises an RNA or an mRNA and the CSR or the sequence encoding the CSR is transiently expressed by the cell.

The disclosure provides a cell comprising a CAR of the disclosure. In some embodiments, the cell further comprises a CSR of the disclosure or a sequence encoding the CSR. In some embodiments, the sequence encoding the CSR comprises DNA. In some embodiments, the sequence encoding the CSR comprises RNA. In some embodiments, the sequence encoding the CSR comprises messenger RNA (mRNA). In some embodiments, the CSR or the sequence encoding the CSR is stably integrated into a genomic locus or loci of the cell. In some embodiments, the CSR or the sequence encoding the CSR is not stably integrated into a genomic locus or loci of the cell. In some embodiments, the CSR or the sequence encoding the CSR is stably expressed by the cell. In some embodiments, the CSR or the sequence encoding the CSR is transiently expressed by the cell. In some embodiments, the CSR or the sequence encoding the CSR comprises an RNA or an mRNA and the CSR or the sequence encoding the CSR is transiently expressed by the cell.

Transposons of the disclosure may comprise piggyBac transposons. In certain embodiments of this method, the transposon is a plasmid DNA transposon with a sequence encoding the chimeric antigen receptor flanked by two cis-regulatory insulator elements. In certain embodiments, the transposon is a piggyBac or a piggyBac-like transposon.

Transposase enzymes of the disclosure may include piggyBac transposases or compatible enzymes. Transposase enzymes of the disclosure may include piggyBac-like transposases or compatible enzymes. In certain embodiments, and, in particular, those embodiments wherein the transposon is a piggyBac transposon, the transposase is a piggyBac™ or a Super piggyBac™ (SPB) transposase. In certain embodiments, and, in particular, those embodiments wherein the transposase is a Super piggyBac™ (SPB) transposase, the sequence encoding the transposase is an mRNA sequence.

In certain embodiments of the methods of the disclosure, the transposase enzyme is a piggyBac™ (PB) transposase enzyme. The piggyBac (PB) transposase enzyme may comprise or consist of an amino acid sequence at least 75%, 80%, 85%, 90%, 95%, 99% or any percentage in between identical to:

```
(SEQ ID NO: 14487)
    1 MGSSLDDEHI LSALLQSDDE LVGEDSDSEI SDHVSEDDVQ SDTEEAFIDE VHEVQPTSSG
  61 SEILDEQNVI EQPGSSLASN RILTLPQRTI RGKNKHCWST SKSTRRSRVS ALNIVRSQRG
121 PTRMCRNIYD PLLCFKLFFT DEIISEIVKW TNAEISLKRR ESMTGATFRD TNEDEIYAFF
181 GILVMTAVRK DNHMSTDDLF DRSLSMVYVS VMSRDRFDFL IRCLRMDDKS IRPTLRENDV
241 FTPVRKIWDL FIHQCIQNYT PGAHLTIDEQ LLGFRGRCPF RMYIPNKPSK YGIKILMMCD
301 SGTKYMINGM PYLGRGTQTN GVPLGEYYVK ELSKPVHGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDTLD QMCSVMTCSR KTNRWPMALL YGMINIACIN
481 SFIIYSHNVS SKGEKVQSRK KFMRNLYMSL TSSFMRKRLE APTLKRYLRD NISNILPNEV
541 PGTSDDSTEE PVMKKRTYCT YCPSKIRRKA NASCKKCKKV ICREHNIDMC QSCF.
```

In certain embodiments of the methods of the disclosure, the transposase enzyme is a piggyBac™ (PB) transposase enzyme that comprises or consists of an amino acid sequence having an amino acid substitution at one or more of positions 30, 165, 282, or 538 of the sequence:

```
(SEQ ID NO: 14487)
    1 MGSSLDDEHI LSALLQSDDE LVGEDSDSEI SDHVSEDDVQ SDTEEAFIDE VHEVQPTSSG
  61 SEILDEQNVI EQPGSSLASN RILTLPQRTI RGKNKHCWST SKSTRRSRVS ALNIVRSQRG
121 PTRMCRNIYD PLLCFKLFFT DEIISEIVKW TNAEISLKRR ESMTGATFRD TNEDEIYAFF
181 GILVMTAVRK DNHMSTDDLF DRSLSMVYVS VMSRDRFDFL IRCLRMDDKS IRPTLRENDV
241 FTPVRKIWDL FIHQCIQNYT PGAHLTIDEQ LLGFRGRCPF RMYIPNKPSK YGIKILMMCD
301 SGTKYMINGM PYLGRGTQTN GVPLGEYYVK ELSKPVHGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDTLD QMCSVMTCSR KTNRWPMALL YGMINIACIN
481 SFIIYSHNVS SKGEKVQSRK KFMRNLYMSL TSSFMRKRLE APTLKRYLRD NISNILPNEV
541 PGTSDDSTEE PVMKKRTYCT YCPSKIRRKA NASCKKCKKV ICREHNIDMC QSCF.
```

In certain embodiments, the transposase enzyme is a piggyBac™ (PB) transposase enzyme that comprises or consists of an amino acid sequence having an amino acid substitution at two or more of positions 30, 165, 282, or 538 of the sequence of SEQ ID NO: 14487. In certain embodiments, the transposase enzyme is a piggyBac™ (PB) transposase enzyme that comprises or consists of an amino acid sequence having an amino acid substitution at three or more of positions 30, 165, 282, or 538 of the sequence of SEQ ID NO: 14487. In certain embodiments, the transposase enzyme is a piggyBac™ (PB) transposase enzyme that comprises or consists of an amino acid sequence having an amino acid substitution at each of the following positions 30, 165, 282, and 538 of the sequence of SEQ ID NO: 14487. In certain embodiments, the amino acid substitution at position 30 of the sequence of SEQ ID NO: 14487 is a substitution of a valine (V) for an isoleucine (I). In certain embodiments, the amino acid substitution at position 165 of the sequence of SEQ ID NO: 14487 is a substitution of a serine (S) for a glycine (G). In certain embodiments, the amino acid substitution at position 282 of the sequence of SEQ ID NO: 14487 is a substitution of a valine (V) for a methionine (M). In certain embodiments, the amino acid substitution at position 538 of the sequence of SEQ ID NO: 14487 is a substitution of a lysine (K) for an asparagine (N).

In certain embodiments of the methods of the disclosure, the transposase enzyme is a Super piggyBac™ (sPBo) transposase enzyme. In certain embodiments, the Super piggyBac™ (sPBo) transposase enzymes of the disclosure may comprise or consist of the amino acid sequence of the sequence of SEQ ID NO: 14487 wherein the amino acid substitution at position 30 is a substitution of a valine (V) for an isoleucine (I), the amino acid substitution at position 165 is a substitution of a serine (S) for a glycine (G), the amino acid substitution at position 282 is a substitution of a valine (V) for a methionine (M), and the amino acid substitution at position 538 is a substitution of a lysine (K) for an asparagine (N). In certain embodiments, the Super piggyBac™ (sPBo) transposase enzyme may comprise or consist of an amino acid sequence at least 75%, 80%, 85%, 90%, 95%, 99% or any percentage in between identical to:

```
(SEQ ID NO: 14484)
    1 MGSSLDDEHI LSALLQSDDE LVGEDSDSEV SDHVSEDDVQ SDTEEAFIDE VHEVQPTSSG
  61 SEILDEQNVI EQPGSSLASN RILTLPQRTI RGKNKHCWST SKSTRRSRVS ALNIVRSQRG
121 PTRMCRNIYD PLLCFKLFFT DEIISEIVKW TNAEISLKRR ESMTSATFRD TNEDEIYAFF
181 GILVMTAVRK DNHMSTDDLF DRSLSMVYVS VMSRDREDFL IRCLRMDDKS IRPTLRENDV
241 FTPVRKIWDL FIHQCIQNYT PGAHLTIDEQ LLGFRGRCPF RVYIPNKPSK YGIKILMMCD
301 SGTKYMINGM PYLGRGTQTN GVPLGEYYVK ELSKPVHGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDTLD QMCSVMTCSR KTNRWPMALL YGMINIACIN
481 SFIIYSHNVS SKGEKVQSRK KFMRNLYMSL TSSFMRKRLE APTLKRYLRD NISNILPKEV
541 PGTSDDSTEE PVMKKRTYCT YCPSKIRRKA NASCKKCKKV ICREHNIDMC QSCF.
```

In certain embodiments of the methods of the disclosure, the transposase enzyme is a TcBuster transposon and wherein the transposase enzyme is a TcBuster transposase enzyme. In some embodiments, the TcBuster transposase enzyme is a hyperactive TcBuster transposase enzyme. In some embodiments, the TcBuster transposase enzyme comprises a sequence having at least 75% identity to:

(SEQ ID NO: 17900)
MMLNWLKSGK LESQSQEQSS CYLENSNCLP PTLDSTDIIG
EENKAGTTSR KKRKYDEDYL NEGFTWTGDK DEPNGLCVIC
EQVVNNSSLN PAKLKRHLDT KEFILKGKSE YEKRKCNELN
QKKETFERYV RDDNKNLLKA SYLVSLRIAK QGEAYTIAEK
LIKPCTKDLT TCVFGEKFAS KVDIVPLSDI TISRRIEDMS
YFCEAVLVNR LENAKGGFTL QMDESTDVAG LAILLVEVRY
IHESSFEEDM LFCKALPTOT IGEE1FNLLN AYEEKHSIPW
NLCYHICIDG AKAMVGVIKG VIARIKKLVP DIKASHCCLH
RHALAVKRIP NALHEVLNDA VKMINFIKSR PLNARVFALL
CDDLGSLHKN LLLHTEVRWL SRGKVLIREW ELRDEIRIFF
NEREFAGKLN DTSWLQNLAY IADIFSYLNE VNLSLQGPNS
TIFKVNSRIN SIKSKLKLWE ECITKNNTEC FANLNDFLET
SNIALDPNIK SNILEHLNGL KNTFLEYFPP TCNNISWVEN
PFNECGNVDT LPIKEREQLI DIRTDITLKS SFVPDGIGPF
WIKLMDEFPE ISKRAVKELM PEVITYLCEK SESVYVAIKT
KYRNRLDAED DMRLQLTTIH PDIDNLCNNK QAQKSH.

In certain embodiments of the methods of the disclosure, the transposase enzyme is a Sleeping Beauty transposon and the transposase enzyme is a Sleeping Beauty transposase enzyme. In some embodiments, the Sleeping Beauty transposase enzyme comprises the sequence of SEQ ID NO: 14485. In some embodiments, the Sleeping Beauty transposase enzyme is a hyperactive Sleeping Beauty transposase (SB100X). In some embodiments, the hyperactive Sleeping Beauty transposase (SB100X) comprises the sequence of SEQ ID NO: 14486.

In certain embodiments of the methods of the disclosure, the transposase enzyme is a Helraiser transposon and wherein the transposase enzyme is a Helraiser transposase enzyme. In some embodiments, the Helraiser transposase enzyme comprises the sequence of SEQ ID NO: 14501.

In certain embodiments of the methods of the disclosure, the transposase enzyme is a Tol2 transposon and wherein the transposase enzyme is a Tol2 transposase enzyme. In some embodiments, the Tol2 transposase enzyme comprises the sequence of SEQ ID NO: 14502.

The disclosure provides a vector comprising a CARTyrin of the disclosure. In certain embodiments, the vector is a viral vector. The vector may be a recombinant vector.

Viral vectors of the disclosure may comprise a sequence isolated or derived from a retrovirus, a lentivirus, an adenovirus, an adeno-associated virus or any combination thereof. The viral vector may comprise a sequence isolated or derived from an adeno-associated virus (AAV). The viral vector may comprise a recombinant AAV (rAAV). Exemplary adeno-associated viruses and recombinant adeno-associated viruses of the disclosure comprise two or more inverted terminal repeat (ITR) sequences located in cis next to a sequence encoding a Centyrin or CARTyrin of the disclosure. Exemplary adeno-associated viruses and recombinant adeno-associated viruses of the disclosure include, but are not limited to all serotypes (e.g. AAV1, AAV2, AAV3, AAV4, AAV5, AAV6, AAV7, AAV8, and AAV9). Exemplary adeno-associated viruses and recombinant adeno-associated viruses of the disclosure include, but are not limited to, self-complementary AAV (scAAV) and AAV hybrids containing the genome of one serotype and the capsid of another serotype (e.g. AAV2/5, AAV-DJ and AAV-DJ8). Exemplary adeno-associated viruses and recombinant adeno-associated viruses of the disclosure include, but are not limited to, rAAV-LK03.

Viral vectors of the disclosure may comprise a selection gene. The selection gene may encode a gene product essential for cell viability and survival. The selection gene may encode a gene product essential for cell viability and survival when challenged by selective cell culture conditions. Selective cell culture conditions may comprise a compound harmful to cell viability or survival and wherein the gene product confers resistance to the compound. Exemplary selection genes of the disclosure may include, but are not limited to, neo (conferring resistance to neomycin), TYMS (encoding Thymidylate Synthetase), MGMT (encoding O(6)-methylguanine-DNA methyltransferase), multidrug resistance gene (MDR1), ALDH1 (encoding Aldehyde dehydrogenase 1 family, member A1), FRANCF, RAD51C (encoding RAD51 Paralog C), GCS (encoding glucosylceramide synthase), NKX2.2 (encoding NK2 Homeobox 2) or any combination thereof.

Viral vectors of the disclosure may comprise an inducible proapoptotic polypeptide comprising (a) a ligand binding region, (b) a linker, and (c) a proapoptotic polypeptide, wherein the inducible proapoptotic polypeptide does not comprise a non-human sequence. In certain embodiments, the non-human sequence comprises a restriction site. In certain embodiments, the ligand binding region may be a multimeric ligand binding region. Inducible proapoptotic polypeptides of the disclosure may also be referred to as an “iC9 safety switch”. In certain embodiments, viral vectors of the disclosure may comprise an inducible caspase polypeptide comprising (a) a ligand binding region, (b) a linker, and (c) a caspase polypeptide, wherein the inducible proapoptotic polypeptide does not comprise a non-human sequence. In certain embodiments, viral vectors of the disclosure may comprise an inducible caspase polypeptide comprising (a) a ligand binding region, (b) a linker, and (c) a caspase polypeptide, wherein the inducible proapoptotic polypeptide does not comprise a non-human sequence. In certain embodiments, viral vectors of the disclosure may comprise an inducible caspase polypeptide comprising (a) a ligand binding region, (b) a linker, and (c) a truncated caspase 9 polypeptide, wherein the inducible proapoptotic polypeptide does not comprise a non-human sequence. In certain embodiments of the inducible proapoptotic polypeptides, inducible caspase polypeptides or truncated caspase 9 polypeptides of the disclosure, the ligand binding region may comprise a FK506 binding protein 12 (FKBP12) polypeptide. In certain embodiments, the amino acid sequence of the ligand binding region that comprise a FK506 binding protein 12 (FKBP12) polypeptide may comprise a modification at position 36 of the sequence. The modification may be a substitution of valine (V) for phenylalanine (F) at position 36 (F36V). In certain embodiments, the FKBP12 polypeptide is encoded by an amino acid sequence comprising

(SEQ ID NO: 18026)
GVQVETISPGDGRTFPKRGQTCVVHYTGMLEDGKKVDSSRDRNKPFKFML
GKQEVIRGWEEGVAQMSVGQRAKLTISPDYAYGATGHPGIIPPHATLVFD
VELLKLE.

In certain embodiments, the FKBP12 polypeptide is encoded by a nucleic acid sequence comprising

(SEQ ID NO: 18027)
GGGGTCCAGGTCGAGACTATTTCACCAGGGGATGGGCGAACATTTCCAAA
AAGGGGCCAGACTTGCGTCGTGCATTACACCGGGATGCTGGAGGACGGGA
AGAAAGTGGACAGCTCCAGGGATCGCAACAAGCCCTTCAAGTTCATGCTG
GGAAAGCAGGAAGTGATCCGAGGATGGGAGGAAGGCGTGGCACAGATGTC
AGTCGGCCAGCGGGCCAAACTGACCATTAGCCCTGACTACGCTTATGGAG
CAACAGGCCACCCAGGGATCATTCCCCCTCATGCCACCCTGGTCTTCGAT
GTGGAACTGCTGAAGCTGGAG.

In certain embodiments, the induction agent specific for the ligand binding region may comprise a FK506 binding protein 12 (FKBP12) polypeptide having a substitution of valine (V) for phenylalanine (F) at position 36 (F36V) comprises AP20187 and/or AP1903, both synthetic drugs.

In certain embodiments of the inducible proapoptotic polypeptides, inducible caspase polypeptides or truncated caspase 9 polypeptides of the disclosure, the linker region is encoded by an amino acid comprising GGGGS (SEQ ID NO: 18028) or a nucleic acid sequence comprising GGAGGAGGAGGATCC (SEQ ID NO: 18029). In certain embodiments, the nucleic acid sequence encoding the linker does not comprise a restriction site.

In certain embodiments of the truncated caspase 9 polypeptides of the disclosure, the truncated caspase 9 polypeptide is encoded by an amino acid sequence that does not comprise an arginine (R) at position 87 of the sequence. Alternatively, or in addition, in certain embodiments of the inducible proapoptotic polypeptides, inducible caspase polypeptides or truncated caspase 9 polypeptides of the disclosure, the truncated caspase 9 polypeptide is encoded by an amino acid sequence that does not comprise an alanine (A) at position 282 the sequence. In certain embodiments of the inducible proapoptotic polypeptides, inducible caspase polypeptides or truncated caspase 9 polypeptides of the disclosure, the truncated caspase 9 polypeptide is encoded by an amino acid comprising

(SEQ ID NO: 18030)
GFGDVGALESLRGNADLAYILSMEPCGHCLIINNVNFCRESGLRTRTGSN
IDCEKLRRRFSSLHFMVEVKGDLTAKKMVLALLELAQQDHGALDCCVVVI
LSHGCQASHLQFPGAVYGTDGCPVSVEKIVNIFNGTSCPSLGGKPKLFFI
QACGGEQKDHGFEVASTSPEDESPGSNPEPDATPFQEGLRTFDQLDAISS
LPTPSDIFVSYSTFPGFVSWRDPKSGSWYVETLDDIFEQWAHSEDLQSLL
LRVANAVSVKGIYKQMPGCFNFLRKKLFFKTS

or a nucleic acid sequence comprising

(SEQ ID NO: 18031)
GGATTTGGGGACGTGGGGGCCCTGGAGTCTCTGCGAGGAAATGCCGATCT
GGCTTACATCCTGAGCATGGAACCCTGCGGCCACTGTCTGATCATTAACA
ATGTGAACTTCTGCAGAGAAAGCGGACTGCGAACACGGACTGGCTCCAAT
ATTGACTGTGAGAAGCTGCGGAGAAGGTTCTCTAGTCTGCACTTTATGGT
CGAAGTGAAAGGGGATCTGACCGCCAAGAAAATGGTGCTGGCCCTGCTGG
AGCTGGCTCAGCAGGACCATGGAGCTCTGGATTGCTGCGTGGTCGTGATC
CTGTCCCACGGGTGCCAGGCTTCTCATCTGCAGTTCCCCGGAGCAGTGTA
CGGAACAGACGGCTGTCCTGTCAGCGTGGAGAAGATCGTCAACATCTTCA
ACGGCACTTCTTGCCCTAGTCTGGGGGGAAAGCCAAAACTGTTCTTTATC
CAGGCCTGTGGCGGGGAACAGAAAGATCACGGCTTCGAGGTGGCCAGCAC
CAGCCCTGAGGACGAATCACCAGGGAGCAACCCTGAACCAGATGCAACTC
CATTCCAGGAGGGACTGAGGACCTTTGACCAGCTGGATGCTATCTCAAGC
CTGCCCACTCCTAGTGACATTTTCGTGTCTTACAGTACCTTCCCAGGCTT
TGTCTCATGGCGCGATCCCAAGTCAGGGAGCTGGTACGTGGAGACACTGG
ACGACATCTTTGAACAGTGGGCCCATTCAGAGGACCTGCAGAGCCTGCTG
CTGCGAGTGGCAAACGCTGTCTCTGTGAAGGGCATCTACAAACAGATGCC
CGGGTGCTTCAATTTTCTGAGAAAGAAACTGTTCTTTAAGACTTCC.

In certain embodiments of the inducible proapoptotic polypeptides, wherein the polypeptide comprises a truncated caspase 9 polypeptide, the inducible proapoptotic polypeptide is encoded by an amino acid sequence comprising

(SEQ ID NO: 18032)
GVQVETISPGDGRTFPKRGQTCVVHYTGMLEDGKKVDSSRDRNKPFKFML
GKQEVIRGWEEGVAQMSVGQRAKLTISPDYAYGATGHPGIIPPHATLVFD
VELLKLEGGGGSGFGDVGALESLRGNADLAYILSMEPCGHCLIINNVNFC
RESGLRTRTGSNIDCEKLRRRFSSLHFMVEVKGDLTAKKMVLALLELAQQ
DHGALDCCVVVILSHGCQASHLQFPGAVYGTDGCPVSVEKIVNIFNGTSC
PSLGGKPKLFFIQACGGEQKDHGFEVASTSPEDESPGSNPEPDATPFQEG
LRTFDQLDAISSLPTPSDIFVSYSTFPGFVSWRDPKSGSWYVETLDDIFE
QWAHSEDLQSLLLRVANAVSVKGIYKQMPGCFNFLRKKLFFKTS

or the nucleic acid sequence comprising

(SEQ ID NO: 18033)
ggggtccaggtcgagactatttcaccaggggatgggcgaacatttccaaa
aaggggccagacttgcgtcgtgcattacaccgggatgctggaggacggga
agaaagtggacagctccagggatcgcaacaagcccttcaagttcatgctg
ggaaagcaggaagtgatccgaggatgggaggaaggcgtggcacagatgtc
agtcggccagcgggccaaactgaccattagccctgactacgcttatggag
caacaggccacccagggatcattccccctcatgccaccctggtcttcgat
gtggaactgctgaagctggagggaggaggaggatccggatttggggacgt
gggggccctggagtctctgcgaggaaatgccgatctggcttacatcctga
gcatggaaccctgcggccactgtctgatcattaacaatgtgaacttctgc
agagaaagcggactgcgaacacggactggctccaatattgactgtgagaa
gctgcggagaaggttctctagtctgcactttatggtcgaagtgaaagggg
atctgaccgccaagaaaatggtgctggccctgctggagctggctcagcag
gaccatggagctctggattgctgcgtggtcgtgatcctgtcccacgggtg
ccaggcttctcatctgcagttccccggagcagtgtacggaacagacggct
gtcctgtcagcgtggagaagatcgtcaacatcttcaacggcacttcttgc
cctagtctggggggaaagccaaaactgttctttatccaggcctgtggcgg
ggaacagaaagatcacggcttcgaggtggccagcaccagccctgaggacg
aatcaccagggagcaaccctgaaccagatgcaactccattccaggaggga
ctgaggacctttgaccagctggatgctatctcaagcctgcccactcctag
tgacattacgtgtcttacagtaccttcccaggctttgtctcatggcgcga
tcccaagtcagggagctggtacgtggagacactggacgacatctttgaac
agtgggcccattcagaggacctgcagagcctgctgctgcgagtggcaaac
gctgtctctgtgaagggcatctacaaacagatgcccgggtgcttcaattt
tctgagaaagaaactgttctttaagacttcc.

Viral vectors of the disclosure may comprise at least one self-cleaving peptide. In some embodiments, the vector may comprise at least one self-cleaving peptide and wherein a self-cleaving peptide is located between a CARtyrin and a selection gene. In some embodiments, the vector may comprise at least one self-cleaving peptide and wherein a first self-cleaving peptide is located upstream of a CARtyrin and a second self-cleaving peptide is located downstream of a CARtyrin. Viral vectors of the disclosure may comprise at least one self-cleaving peptide(s) located, for example, between one or more of a CARTyrin, CAR or CAR of the disclosure and an inducible proapoptotic polypeptide of the disclosure. Viral vectors of the disclosure may comprise at least two self-cleaving peptide(s), a first self-cleaving peptide located, for example, upstream or immediately upstream of an inducible proapoptotic polypeptide of the disclosure and a second first self-cleaving peptide located, for example, downstream or immediately upstream of an inducible proapoptotic polypeptide of the disclosure. The self-cleaving peptide may comprise, for example, a T2A peptide, GSG-T2A peptide, an E2A peptide, a GSG-E2A peptide, an F2A peptide, a GSG-F2A peptide, a P2A peptide, or a GSG-P2A peptide. A T2A peptide may comprise an amino acid sequence comprising EGRGSLLTCGDVEENPGP (SEQ ID NO: 18034) or a sequence having at least 70%, 80%, 90%, 95%, or 99% identity to the amino acid sequence comprising EGRGSLLTCGDVEENPGP (SEQ ID NO: 18035). A GSG-T2A peptide may comprise an amino acid sequence comprising GSGEGRGSLLTCGDVEENPGP (SEQ ID NO: 18036) or a sequence having at least 70%, 80%, 90%, 95%, or 99% identity to the amino acid sequence comprising GSGEGRGSLLTCGDVEENPGP (SEQ ID NO: 18037). A GSG-T2A peptide may comprise a nucleic acid sequence comprising

(SEQ ID NO: 18038)
ggatctggagagggaaggggaagcctgctgacctgtggagacgtggagga
aaacccaggacca.

An E2A peptide may comprise an amino acid sequence comprising QCTNYALLKLAGDVESNPGP (SEQ ID NO: 18039) or a sequence having at least 70%, 80%, 90%, 95%, or 99% identity to the amino acid sequence comprising QCTNYALLKLAGDVESNPGP (SEQ ID NO: 18040). A GSG-E2A peptide may comprise an amino acid sequence comprising GSGQCTNYALLKLAGDVESNPGP (SEQ ID NO: 18041) or a sequence having at least 70%, 80%, 90%, 95%, or 99% identity to the amino acid sequence comprising GSGQCTNYALLKLAGDVESNPGP (SEQ ID NO: 18042). An F2A peptide may comprise an amino acid sequence comprising VKQTLNFDLLKLAGDVESNPGP (SEQ ID NO: 18043) or a sequence having at least 70%, 80%, 90%, 95%, or 99% identity to the amino acid sequence comprising VKQTLNFDLLKLAGDVESNPGP (SEQ ID NO: 18044). A GSG-F2A peptide may comprise an amino acid sequence comprising GSGVKQTLNFDLLKLAGDVESNPGP (SEQ ID NO: 18045) or a sequence having at least 70%, 80%, 90%, 95%, or 99% identity to the amino acid sequence comprising GSGVKQTLNFDLLKLAGDVESNPGP (SEQ ID NO: 18046). A P2A peptide may comprise an amino acid sequence comprising ATNFSLLKQAGDVEENPGP (SEQ ID NO: 18047) or a sequence having at least 70%, 80%, 90%, 95%, or 99% identity to the amino acid sequence comprising ATNFSLLKQAGDVEENPGP (SEQ ID NO: 18048). A GSG-P2A peptide may comprise an amino acid sequence comprising GSGATNFSLLKQAGDVEENPGP (SEQ ID NO: 18049) or a sequence having at least 70%, 80%, 90%, 95%, or 99% identity to the amino acid sequence comprising GSGATNFSLLKQAGDVEENPGP (SEQ ID NO: 18050).

The disclosure provides a vector comprising the CARTyrin of the disclosure. In certain embodiments, the vector is a nanoparticle. Exemplary nanoparticle vectors of the disclosure include, but are not limited to, nucleic acids (e.g. RNA, DNA, synthetic nucleotides, modified nucleotides or any combination thereof), amino acids (L-amino acids, D-amino acids, synthetic amino acids, modified amino acids, or any combination thereof), polymers (e.g. polymersomes), micelles, lipids (e.g. liposomes), organic molecules (e.g. carbon atoms, sheets, fibers, tubes), inorganic molecules (e.g. calcium phosphate or gold) or any combination thereof. A nanoparticle vector may be passively or actively transported across a cell membrane.

Nanoparticle vectors of the disclosure may comprise a selection gene. The selection gene may encode a gene product essential for cell viability and survival. The selection gene may encode a gene product essential for cell viability and survival when challenged by selective cell culture conditions. Selective cell culture conditions may comprise a compound harmful to cell viability or survival and wherein the gene product confers resistance to the compound. Exemplary selection genes of the disclosure may include, but are not limited to, neo (conferring resistance to neomycin), TYMS (encoding Thymidylate Synthetase), MGMT (encoding O(6)-methylguanine-DNA methyltransferase), multidrug resistance gene (MDR1), ALDH1 (encoding Aldehyde dehydrogenase 1 family, member A1), FRANCF, RAD51C (encoding RAD51 Paralog C), GCS (encoding glucosylceramide synthase), NKX2.2 (encoding NK2 Homeobox 2) or any combination thereof.

Nanoparticle vectors of the disclosure may comprise an inducible proapoptotic polypeptide comprising (a) a ligand binding region, (b) a linker, and (c) a proapoptotic polypeptide, wherein the inducible proapoptotic polypeptide does not comprise a non-human sequence. In certain embodiments, the non-human sequence comprises a restriction site. In certain embodiments, the ligand binding region may be a multimeric ligand binding region. Inducible proapoptotic polypeptides of the disclosure may also be referred to as an “iC9 safety switch”. In certain embodiments, nanoparticle vectors of the disclosure may comprise an inducible caspase polypeptide comprising (a) a ligand binding region, (b) a linker, and (c) a caspase polypeptide, wherein the inducible proapoptotic polypeptide does not comprise a non-human sequence. In certain embodiments, nanoparticle vectors of the disclosure may comprise an inducible caspase polypeptide comprising (a) a ligand binding region, (b) a linker, and (c) a caspase polypeptide, wherein the inducible proapoptotic polypeptide does not comprise a non-human sequence. In certain embodiments, nanoparticle vectors of the disclosure may comprise an inducible caspase polypeptide comprising (a) a ligand binding region, (b) a linker, and (c) a truncated caspase 9 polypeptide, wherein the inducible proapoptotic polypeptide does not comprise a non-human sequence. In certain embodiments of the inducible proapoptotic polypeptides, inducible caspase polypeptides or truncated caspase 9 polypeptides of the disclosure, the ligand binding region may comprise a FK506 binding protein 12 (FKBP12) polypeptide. In certain embodiments, the amino acid sequence of the ligand binding region that comprise a FK506 binding protein 12 (FKBP12) polypeptide may comprise a modification at position 36 of the sequence. The modification may be a substitution of valine (V) for phenylalanine (F) at position 36 (F36V). In certain embodiments, the FKBP12 polypeptide is encoded by an amino acid sequence comprising

(SEQ ID NO: 18026)
GVQVETISPGDGRTFPKRGQTCVVHYTGMLEDGKKVDSSRDRNKPFKFML
GKQEVIRGWEEGVAQMSVGQRAKLTISPDYAYGATGHPGIIPPHATLVFD
VELLKLE.

In certain embodiments, the FKBP12 polypeptide is encoded by a nucleic acid sequence comprising

(SEQ ID NO: 18027)
GGGGTCCAGGTCGAGACTATTTCACCAGGGGATGGGCGAACATTTCCAAA
AAGGGGCCAGACTTGCGTCGTGCATTACACCGGGATGCTGGAGGACGGGA
AGAAAGTGGACAGCTCCAGGGATCGCAACAAGCCCTTCAAGTTCATGCTG
GGAAAGCAGGAAGTGATCCGAGGATGGGAGGAAGGCGTGGCACAGATGTC
AGTCGGCCAGCGGGCCAAACTGACCATTAGCCCTGACTACGCTTATGGAG
CAACAGGCCACCCAGGGATCATTCCCCCTCATGCCACCCTGGTCTTCGAT
GTGGAACTGCTGAAGCTGGAG.

In certain embodiments, the induction agent specific for the ligand binding region may comprise a FK506 binding protein 12 (FKBP12) polypeptide having a substitution of valine (V) for phenylalanine (F) at position 36 (F36V) comprises AP20187 and/or AP1903, both synthetic drugs.

In certain embodiments of the inducible proapoptotic polypeptides, inducible caspase polypeptides or truncated caspase 9 polypeptides of the disclosure, the linker region is encoded by an amino acid comprising GGGGS (SEQ ID NO: 18028) or a nucleic acid sequence comprising GGAGGAGGAGGATCC (SEQ ID NO: 18029). In certain embodiments, the nucleic acid sequence encoding the linker does not comprise a restriction site.

In certain embodiments of the truncated caspase 9 polypeptides of the disclosure, the truncated caspase 9 polypeptide is encoded by an amino acid sequence that does not comprise an arginine (R) at position 87 of the sequence. Alternatively, or in addition, in certain embodiments of the inducible proapoptotic polypeptides, inducible caspase polypeptides or truncated caspase 9 polypeptides of the disclosure, the truncated caspase 9 polypeptide is encoded by an amino acid sequence that does not comprise an alanine (A) at position 282 the sequence. In certain embodiments of the inducible proapoptotic polypeptides, inducible caspase polypeptides or truncated caspase 9 polypeptides of the disclosure, the truncated caspase 9 polypeptide is encoded by an amino acid comprising

(SEQ ID NO: 18030)
GFGDVGALESLRGNADLAYILSMEPCGHCLIINNVNFCRESGLRTRTGSN
IDCEKLRRRFSSLHFMVEVKGDLTAKKMVLALLELAQQDHGALDCCVVVI
LSHGCQASHLQFPGAVYGTDGCPVSVEKIVNIFNGTSCPSLGGKPKLFFI
QACGGEQKDHGFEVASTSPEDESPGSNPEPDATPFQEGLRTFDQLDAISS
LPTPSDIFVSYSTFPGFVSWRDPKSGSWYVETLDDIFEQWAHSEDLQSLL
LRVANAVSVKGIYKQMPGCFNFLRKKLFFKTS

or a nucleic acid sequence comprising

(SEQ ID NO: 18031)
GGATTTGGGGACGTGGGGGCCCTGGAGTCTCTGCGAGGAAATGCCGATCT
GGCTTACATCCTGAGCATGGAACCCTGCGGCCACTGTCTGATCATTAACA
ATGTGAACTTCTGCAGAGAAAGCGGACTGCGAACACGGACTGGCTCCAAT
ATTGACTGTGAGAAGCTGCGGAGAAGGTTCTCTAGTCTGCACTTTATGGT
CGAAGTGAAAGGGGATCTGACCGCCAAGAAAATGGTGCTGGCCCTGCTGG
AGCTGGCTCAGCAGGACCATGGAGCTCTGGATTGCTGCGTGGTCGTGATC
CTGTCCCACGGGTGCCAGGCTTCTCATCTGCAGTTCCCCGGAGCAGTGTA
CGGAACAGACGGCTGTCCTGTCAGCGTGGAGAAGATCGTCAACATCTTCA
ACGGCACTTCTTGCCCTAGTCTGGGGGGAAAGCCAAAACTGTTCTTTATC
CAGGCCTGTGGCGGGGAACAGAAAGATCACGGCTTCGAGGTGGCCAGCAC
CAGCCCTGAGGACGAATCACCAGGGAGCAACCCTGAACCAGATGCAACTC
CATTCCAGGAGGGACTGAGGACCTTTGACCAGCTGGATGCTATCTCAAGC
CTGCCCACTCCTAGTGACATTTTCGTGTCTTACAGTACCTTCCCAGGCTT
TGTCTCATGGCGCGATCCCAAGTCAGGGAGCTGGTACGTGGAGACACTGG
ACGACATCTTTGAACAGTGGGCCCATTCAGAGGACCTGCAGAGCCTGCTG
CTGCGAGTGGCAAACGCTGTCTCTGTGAAGGGCATCTACAAACAGATGCC
CGGGTGCTTCAATTTTCTGAGAAAGAAACTGTTCTTTAAGACTTCC.

In certain embodiments of the inducible proapoptotic polypeptides, wherein the polypeptide comprises a truncated caspase 9 polypeptide, the inducible proapoptotic polypeptide is encoded by an amino acid sequence comprising

(SEQ ID NO: 18032)
GVQVETISPGDGRTFPKRGQTCVVHYTGMLEDGKKVDSSRDRNKPFKFML
GKQEVIRGWEEGVAQMSVGQRAKLTISPDYAYGATGHPGIIPPHATLVFD
VELLKLEGGGGSGFGDVGALESLRGNADLAYILSMEPCGHCLIINNVNFC
RESGLRTRTGSNIDCEKLRRRFSSLHFMVEVKGDLTAKKMVLALLELAQQ
DHGALDCCVVVILSHGCQASHLQFPGAVYGTDGCPVSVEKIVNIFNGTSC
PSLGGKPKLFFIQACGGEQKDHGFEVASTSPEDESPGSNPEPDATPFQEG
LRTFDQLDAISSLPTPSDIFVSYSTFPGFVSWRDPKSGSWYVETLDDIFE
QWAHSEDLQSLLLRVANAVSVKGIYKQMPGCFNFLRKKLFFKTS

or the nucleic acid sequence comprising

(SEQ ID NO: 18033)
ggggtccaggtcgagactatttcaccaggggatgggcgaacatttccaaa
aaggggccagacttgcgtcgtgcattacaccgggatgctggaggacggga
agaaagtggacagctccagggatcgcaacaagcccttcaagttcatgctg
ggaaagcaggaagtgatccgaggatgggaggaaggcgtggcacagatgtc
agtcggccagcgggccaaactgaccattagccctgactacgcttatggag
caacaggccacccagggatcattccccctcatgccaccctggtcttcgat
gtggaactgctgaagctggagggaggaggaggatccggatttggggacgt
gggggccctggagtctctgcgaggaaatgccgatctggcttacatcctga
gcatggaaccctgcggccactgtctgatcattaacaatgtgaacttctgc
agagaaagcggactgcgaacacggactggctccaatattgactgtgagaa
gctgcggagaaggttctctagtctgcactttatggtcgaagtgaaagggg
atctgaccgccaagaaaatggtgctggccctgctggagctggctcagcag
gaccatggagctctggattgctgcgtggtcgtgatcctgtcccacgggtg
ccaggcttctcatctgcagttccccggagcagtgtacggaacagacggct
gtcctgtcagcgtggagaagatcgtcaacatcttcaacggcacttcttgc
cctagtctggggggaaagccaaaactgttctttatccaggcctgtggcgg
ggaacagaaagatcacggcttcgaggtggccagcaccagccctgaggacg
aatcaccagggagcaaccctgaaccagatgcaactccattccaggaggga
ctgaggacctttgaccagctggatgctatctcaagcctgcccactcctag
tgacattacgtgtcttacagtaccttcccaggctttgtctcatggcgcga
tcccaagtcagggagctggtacgtggagacactggacgacatctttgaac
agtgggcccattcagaggacctgcagagcctgctgctgcgagtggcaaac
gctgtctctgtgaagggcatctacaaacagatgcccgggtgcttcaattt
tctgagaaagaaactgttattaagacttcc.

Nanoparticle vectors of the disclosure may comprise at least one self-cleaving peptide. In some embodiments, the nanoparticle vector may comprise at least one self-cleaving peptide and wherein a self-cleaving peptide is located between a CARTyrin and the nanoparticle. In some embodiments, the nanoparticle vector may comprise at least one self-cleaving peptide and wherein a first self-cleaving peptide is located upstream of a CARTyrin and a second self-cleaving peptide is located downstream of a CARTyrin. In some embodiments, the nanoparticle vector may comprise at least one self-cleaving peptide and wherein a first self-cleaving peptide is located between a CARTyrin and the nanoparticle and a second self-cleaving peptide is located downstream of the CARTyrin. In some embodiments, the nanoparticle vector may comprise at least one self-cleaving peptide and wherein a first self-cleaving peptide is located between a CARTyrin and the nanoparticle and a second self-cleaving peptide is located downstream of the CARTyrin, for example, between the CARTyrin and a selection gene.

Nanoparticle vectors of the disclosure may comprise at least one self-cleaving peptide(s) located, for example, between one or more Centyrins(s) or CARTyrin(s) of the disclosure and an inducible proapoptotic polypeptide of the disclosure. Nanoparticle vectors of the disclosure may comprise at least two self-cleaving peptide(s), a first self-cleaving peptide located, for example, upstream or immediately upstream of an inducible proapoptotic polypeptide of the disclosure and a second first self-cleaving peptide located, for example, downstream or immediately upstream of an inducible proapoptotic polypeptide of the disclosure. The self-cleaving peptide may comprise, for example, a T2A peptide, GSG-T2A peptide, an E2A peptide, a GSG-E2A peptide, an F2A peptide, a GSG-F2A peptide, a P2A peptide, or a GSG-P2A peptide. A T2A peptide may comprise an amino acid sequence comprising EGRGSLLTCGDVEENPGP (SEQ ID NO: 18034) or a sequence having at least 70%, 80%, 90%, 95%, or 99% identity to the amino acid sequence comprising EGRGSLLTCGDVEENPGP (SEQ ID NO: 18034). A GSG-T2A peptide may comprise an amino acid sequence comprising GSGEGRGSLLTCGDVEENPGP (SEQ ID NO: 18036) or a sequence having at least 70%, 80%, 90%, 95%, or 99% identity to the amino acid sequence comprising GSGEGRGSLLTCGDVEENPGP (SEQ ID NO: 18036). A GSG-T2A peptide may comprise a nucleic acid sequence comprising

(SEQ ID NO: 18038)
ggatctggagagggaaggggaagcctgctgacctgtggagacgtggagga
aaacccaggacca.

An E2A peptide may comprise an amino acid sequence comprising QCTNYALLKLAGDVESNPGP (SEQ ID NO: 18039) or a sequence having at least 70%, 80%, 90%, 95%, or 99% identity to the amino acid sequence comprising QCTNYALLKLAGDVESNPGP (SEQ ID NO: 18039). A GSG-E2A peptide may comprise an amino acid sequence comprising GSGQCTNYALLKLAGDVESNPGP (SEQ ID NO: 18041) or a sequence having at least 70%, 80%, 90%, 95%, or 99% identity to the amino acid sequence comprising GSGQCTNYALLKLAGDVESNPGP (SEQ ID NO: 18041). An F2A peptide may comprise an amino acid sequence comprising VKQTLNFDLLKLAGDVESNPGP (SEQ ID NO: 18043) or a sequence having at least 70%, 80%, 90%, 95%, or 99% identity to the amino acid sequence comprising VKQTLNFDLLKLAGDVESNPGP (SEQ ID NO: 18043). A GSG-F2A peptide may comprise an amino acid sequence comprising GSGVKQTLNFDLLKLAGDVESNPGP (SEQ ID NO: 18045) or a sequence having at least 70%, 80%, 90%, 95%, or 99% identity to the amino acid sequence comprising GSGVKQTLNFDLLKLAGDVESNPGP (SEQ ID NO: 18045). A P2A peptide may comprise an amino acid sequence comprising ATNFSLLKQAGDVEENPGP (SEQ ID NO: 18047) or a sequence having at least 70%, 80%, 90%, 95%, or 99% identity to the amino acid sequence comprising ATNFSLLKQAGDVEENPGP (SEQ ID NO: 18047). A GSG-P2A peptide may comprise an amino acid sequence comprising GSGATNFSLLKQAGDVEENPGP (SEQ ID NO: 18050) or a sequence having at least 70%, 80%, 90%, 95%, or 99% identity to the amino acid sequence comprising GSGATNFSLLKQAGDVEENPGP (SEQ ID NO: 18050).

The disclosure provides a composition comprising a vector of the disclosure. The disclosure provides a cell comprising a CARTyrin of the disclosure. The disclosure provides a cell comprising a transposon of the disclosure. In certain embodiments, the cell comprising a CARTyrin, a transposon, or a vector of the disclosure may express a CARTyrim on the cell surface. The cell may be any type of cell. Preferably, the cell is an immune cell. The immune cell may be a T-cell, a Natural Killer (NK) cell, a Natural Killer (NK)-like cell, Cytokine Induced Killer (CIK) cell), a hematopoeitic progenitor cell, a peripheral blood (PB) derived T cell or an umbilical cord blood (UCB) derived T-cell. Preferably, the immune cell is a T-cell. The T-cell may be an early memory cell, a stem-like T-cell, a T_{SCM}-like cell, a T_{SCM} or a T_{CM}. The T-cell may be a T_{SCM}. The cell may be an artificial antigen presenting cell, which, optionally, may be used to stimulate and expand a modified immune cell or T cell of the disclosure. The cell may be a tumor cell, which, optionally, may be used as an artificial or modified antigen presenting cell.

Modified cells of the disclosure that may be used for adoptive therapy may be autologous or allogeneic.

The disclosure provides a method for expressing a CARTyrin on the surface of a cell, comprising: (a) obtaining a cell population; (b) contacting the cell population to a composition comprising a CARTyrin of the disclosure or a sequence encoding the CARTyrin, under conditions sufficient to transfer the CARTyrin across a cell membrane of at least one cell in the cell population, thereby generating a modified cell population; (c) culturing the modified cell population under conditions suitable for integration of the transposon; and (d) expanding and/or selecting at least one cell from the modified cell population that express the CARTyrin on the cell surface.

In certain embodiments of this method of expressing a CARTyrin, the cell population may comprise leukocytes and/or CD4+ and CD8+ leukocytes. The cell population may comprise CD4+ and CD8+ leukocytes in an optimized ratio. The optimized ratio of CD4+ to CD8+ leukocytes does not naturally occur in vivo. The cell population may comprise a tumor cell.

In certain embodiments of this method of expressing a CARTyrin, a transposon or vector comprises the CARTyrin or the sequence encoding the CARTyrin. In certain embodiments, a transposon comprises an anti-PSMA CARTyrin or the sequence encoding an anti-PSMA CARTyrin. In certain embodiments, the transposon comprises a piggyBac transposon. In certain embodiments, the transposon further comprises a composition comprising a plasmid comprising a sequence encoding a transposase enzyme. In certain embodiments, including those embodiments wherein the transposase is a piggyBac transposase, the transpose enzyme is an mRNA sequence. In certain embodiments, the piggyBac transposase comprises an amino acid sequence comprising SEQ ID NO: 18017. In certain embodiments, the piggyBac transposase is a hyperactive variant and wherein the hyperactive variant comprises an amino acid substitution at one or more of positions 30, 165, 282 and 538 of SEQ ID NO: 18017. In certain embodiments, the amino acid substitution at position 30 of SEQ ID NO: 18017 is a substitution of a valine (V) for an isoleucine (I) (I30V). In certain embodiments, the amino acid substitution at position 165 of SEQ ID NO: 18017 is a substitution of a serine (S) for a glycine (G) (G165S). In certain embodiments, wherein the amino acid substitution at position 282 of SEQ ID NO: 18017 is a substitution of a valine (V) for a methionine (M) (M282V). In certain embodiments, the amino acid substitution at position 538 of SEQ ID NO: 18017 is a substitution of a lysine (K) for an asparagine (N) (N538K). In certain embodiments, the transposase is a Super piggyBac (sPBo) transposase. In certain embodiments, the Super piggyBac (sPBo) transposase comprises an amino acid sequence comprising SEQ ID NO: 14484.

The disclosure provides a vector comprising a CARTyrin or a sequence encoding a CARTyrin of the disclosure. In certain embodiments, the vector comprises an anti-PSMA CARTyrin or a sequence encoding an anti-PSMA CARTyrin of the disclosure.

In certain embodiments of this method of expressing a CARTyrin, the conditions sufficient to transfer the sequence encoding the CARTyrin across a cell membrane of at least one cell in the cell population comprise nucleofection.

In certain embodiments of this method of expressing a CARTyrin, wherein the conditions sufficient to transfer the sequence encoding the CARTyrin across a cell membrane of at least one cell in the cell population comprise at least one of an application of one or more pulses of electricity at a specified voltage, a buffer, and one or more supplemental factor(s). In certain embodiments, the buffer may comprise PBS, HBSS, OptiMEM, BTXpress, Amaxa Nucleofector, Human T cell nucleofection buffer or any combination thereof. In certain embodiments, the one or more supplemental factor(s) may comprise (a) a recombinant human cytokine, a chemokine, an interleukin or any combination thereof; (b) a salt, a mineral, a metabolite or any combination thereof, (c) a cell medium; (d) an inhibitor of cellular DNA sensing, metabolism, differentiation, signal transduction, one or more apoptotic pathway(s) or combinations thereof; and (e) a reagent that modifies or stabilizes one or more nucleic acids. The recombinant human cytokine, the chemokine, the interleukin or any combination thereof may comprise IL2, IL7, IL12, IL15, IL21, IL1, IL3, IL4, IL5, IL6, IL8, CXCL8, IL9, IL10, IL11, IL13, IL14, IL16, IL17, IL18, IL19, IL20, IL22, IL23, IL25, IL26, IL27, IL28, IL29, IL30, IL31, IL32, IL33, IL35, IL36, GM-CSF, IFN-gamma, IL-1 alpha/IL-1F1, IL-1 beta/IL-1F2, IL-12 p70, IL-12/IL-35 p35, IL-13, IL-17/IL-17A, IL-17A/F Heterodimer, IL-17F, IL-18/IL-1F4, IL-23, IL-24, IL-32, IL-32 beta, IL-32 gamma, IL-33, LAP (TGF-beta 1), Lymphotoxin-alpha/TNF-beta, TGF-beta, TNF-alpha, TRANCE/TNFSF11/RANK L or any combination thereof. The salt, the mineral, the metabolite or any combination thereof may comprise HEPES, Nicotinamide, Heparin, Sodium Pyruvate, L-Glutamine, MEM Non-Essential Amino Acid Solution, Ascorbic Acid, Nucleosides, FBS/FCS, Human serum, serum-substitute, anti-biotics, pH adjusters, Earle's Salts, 2-Mercaptoethanol, Human transferrin, Recombinant human insulin, Human serum albumin, Nucleofector PLUS Supplement, KCL, MgCl2, Na2HPO4, NAH2PO4, Sodium lactobionate, Manitol, Sodium succinate, Sodium Chloride, CINa, Glucose, Ca(NO3)2, Tris/HCl, K2HPO4, KH2PO4, Polyethylenimine, Poly-ethylene-glycol, Poloxamer 188, Poloxamer 181, Poloxamer 407, Poly-vinylpyrrolidone, Pop313, Crown-5, or any combination thereof. The cell medium may comprise PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium or any combination thereof. The inhibitor of cellular DNA sensing, metabolism, differentiation, signal transduction, one or more apoptotic pathway(s) or combinations thereof comprise inhibitors of TLR9, MyD88, IRAK, TRAF6, TRAF3, IRF-7, NF-KB, Type 1 Interferons, pro-inflammatory cytokines, cGAS, STING, Sec5, TBK1, IRF-3, RNA pol III, RIG-1, IPS-1, FADD, RIP1, TRAF3, AIM2, ASC, Caspase1, Pro-IL1B, PI3K, Akt, Wnt3A, inhibitors of glycogen synthase kinase-3β (GSK-β) (e.g. TWS119), Bafilomycin, Chloroquine, Quinacrine, AC-YVAD-CMK, Z-VAD-FMK, Z-IETD-FMK or any combination thereof. The reagent that modifies or stabilizes one or more nucleic acids comprises a pH modifier, a DNA-binding protein, a lipid, a phospholipid, CaPO4, a net neutral charge DNA binding peptide with or without a NLS sequence, a TREX1 enzyme or any combination thereof.

In certain embodiments of this method of expressing a CARTyrin, the conditions suitable for integration of the CARTyrin or a sequence encoding the CARTyrin of the disclosure comprise at least one of a buffer and one or more supplemental factor(s). In certain embodiments, a transposon or vector of the disclosure comprise the CARTyrin or a sequence encoding the CARTyrin of the disclosure. In certain embodiments, the buffer may comprise PBS, HBSS, OptiMEM, BTXpress, Amaxa Nucleofector, Human T cell nucleofection buffer or any combination thereof. In certain embodiments, the one or more supplemental factor(s) may comprise (a) a recombinant human cytokine, a chemokine, an interleukin or any combination thereof; (b) a salt, a mineral, a metabolite or any combination thereof; (c) a cell medium; (d) an inhibitor of cellular DNA sensing, metabolism, differentiation, signal transduction, one or more apoptotic pathway(s) or combinations thereof; and (e) a reagent that modifies or stabilizes one or more nucleic acids. The recombinant human cytokine, the chemokine, the interleukin or any combination thereof may comprise IL2, IL7, IL12, IL15, IL21, IL1, IL3, IL4, IL5, IL6, IL8, CXCL8, IL9, IL10, IL11, IL13, IL14, IL16, IL17, IL18, IL19, IL20, IL22, IL23, IL25, IL26, IL27, IL28, IL29, IL30, IL31, IL32, IL33, IL35, IL36, GM-CSF, IFN-gamma, IL-1 alpha/IL-1F1, IL-1 beta/IL-1F2, IL-12 p70, IL-12/IL-35 p35, IL-13, IL-17/IL-17A, IL-17A/F Heterodimer, IL-17F, IL-18/IL-1F4, IL-23, IL-24, IL-32, IL-32 beta, IL-32 gamma, IL-33, LAP (TGF-beta 1), Lymphotoxin-alpha/TNF-beta, TGF-beta, TNF-alpha, TRANCE/TNFSF11/RANK L or any combination thereof. The salt, the mineral, the metabolite or any combination thereof may comprise HEPES, Nicotinamide, Heparin, Sodium Pyruvate, L-Glutamine, MEM Non-Essential Amino Acid Solution, Ascorbic Acid, Nucleosides, FBS/FCS, Human serum, serum-substitute, anti-biotics, pH adjusters, Earle's Salts, 2-Mercaptoethanol, Human transferrin, Recombinant human insulin, Human serum albumin, Nucleofector PLUS Supplement, KCL, MgCl2, Na2HPO4, NAH2PO4, Sodium lactobionate, Manitol, Sodium succinate, Sodium Chloride, CINa, Glucose, Ca(NO3)2, Tris/HCl, K2HPO4, KH2PO4, Polyethylenimine, Poly-ethylene-glycol, Poloxamer 188, Poloxamer 181, Poloxamer 407, Poly-vinylpyrrolidone, Pop313, Crown-5, or any combination thereof. The cell medium may comprise PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium or any combination thereof. The inhibitor of cellular DNA sensing, metabolism, differentiation, signal transduction, one or more apoptotic pathway(s) or combinations thereof comprise inhibitors of TLR9, MyD88, IRAK, TRAF6, TRAF3, IRF-7, NF-KB, Type 1 Interferons, pro-inflammatory cytokines, cGAS, STING, Sec5, TBK1, IRF-3, RNA pol III, RIG-1, IPS-1, FADD, RIP1, TRAF3, AIM2, ASC, Caspase1, Pro-IL1B, PI3K, Akt, Wnt3A, inhibitors of glycogen synthase kinase-3β (GSK-3β) (e.g. TWS119), Bafilomycin, Chloroquine, Quinacrine, AC-YVAD-CMK, Z-VAD-FMK, Z-IETD-FMK or any combination thereof. The reagent that modifies or stabilizes one or more nucleic acids comprises a pH modifier, a DNA-binding protein, a lipid, a phospholipid, CaPO4, a net neutral charge DNA binding peptide with or without a NLS sequence, a TREX1 enzyme or any combination thereof.

In certain embodiments of this method of expressing a CARTyrin, the expansion and selection steps occur sequentially. The expansion may occur prior to selection. The expansion may occur following selection, and, optionally, a further (i.e. second) selection may occur following expansion.

In certain embodiments of this method of expressing a CARTyrin, the expansion and selection steps may occur simultaneously.

In certain embodiments of this method of expressing a CARTyrin, the expansion may comprise contacting at least one cell of the modified cell population with an antigen to stimulate the at least one cell through the CARTyrin, thereby generating an expanded cell population. The antigen may be presented on the surface of a substrate. The substrate may have any form, including, but not limited to a surface, a well, a bead or a plurality thereof, and a matrix. The substrate may further comprise a paramagnetic or magnetic component. In certain embodiments of this method of expressing a CARTyrin, the antigen may be presented on the surface of a substrate, wherein the substrate is a magnetic bead, and wherein a magnet may be used to remove or separate the magnetic beads from the modified and expanded cell population. The antigen may be presented on the surface of a cell or an artificial antigen presenting cell. Artificial antigen presenting cells of the disclosure may include, but are not limited to, tumor cells and stem cells.

In certain embodiments of this method of expressing a CARTyrin, wherein the transposon or vector comprises a selection gene and wherein the selection step comprises contacting at least one cell of the modified cell population with a compound to which the selection gene confers resistance, thereby identifying a cell expressing the selection gene as surviving the selection and identifying a cell failing to express the selection gene as failing to survive the selection step.

In certain embodiments of this method of expressing a CARTyrin, the expansion and/or selection steps may proceed for a period of 10 to 14 days, inclusive of the endpoints.

The disclosure provides a composition comprising the modified, expanded and selected cell population of the methods of the disclosure.

The disclosure provides a method of treating cancer in a subject in need thereof, comprising administering to the subject a composition of the disclosure, wherein the CARTyrin specifically binds to an antigen on a tumor cell. In certain embodiments, the tumor cell may be a malignant tumor cell. In certain embodiments, comprising administering to the subject the composition comprising a modified cell or cell population of the disclosure, the cell or cell population may be autologous. In certain embodiments, comprising administering to the subject the composition comprising a modified cell or cell population of the disclosure, the cell or cell population may be allogeneic.

The disclosure provides a method of treating cancer in a subject in need thereof, comprising administering to the subject a composition of the disclosure, wherein the anti-PSMA CARTyrin specifically binds to a PSMA antigen on a tumor cell or a component of a vasculature of a tumor cell. In certain embodiments, the tumor cell is a prostate cell. In certain embodiments, the tumor cell may be a malignant tumor cell. In certain embodiments, comprising administering to the subject the composition comprising a modified cell or cell population of the disclosure, the cell or cell population may be autologous. In certain embodiments, comprising administering to the subject the composition comprising a modified cell or cell population of the disclosure, the cell or cell population may be allogeneic.

Methods of modifying a cell therapy of the disclosure may be used to terminate or dampen a therapy in response to, for example, a sign of recovery or a sign of decreasing disease severity/progression, a sign of disease remission/cessation, and/or the occurrence of an adverse event. Cell therapies of the disclosure may be resumed by inhibiting the induction agent should a sign or symptom of the disease reappear or increase in severity and/or an adverse event is resolved.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram depicting a piggyBac CARTyrin P-PSMA-101 plasmid of 7738 base pairs that includes an EF1α promoter, a safety switch (iC9), a PSMA CARTyrin and a selection gene casette.

FIG. 2 is a schematic diagram depicting a piggyBac CARTyrin that includes a P-PSMA-101 transposon comprising a PSMA CARTyrin (comprising a CD8α signal peptide, an anti-PSMA Centyrin, a CD8α spacer, a CD8α transmembrane sequence, a 4-1BB costimulatory domain and a CD3ζ costimulatory domain).

FIG. 3A is a schematic diagram of the amino acid sequence of a P-PSMA5-101 construct of the disclosure.

FIG. 3B is a schematic diagram of the nucleic acid sequence of a P-PSMA5-101 construct of the disclosure.

FIG. 3C is a schematic diagram of the nucleic acid sequence of a P-PSMA5-101 construct of the disclosure.

FIG. 4A is a schematic diagram of the amino acid sequence of a P-PSMA8-101 construct of the disclosure.

FIG. 4B is a schematic diagram of the amino acid sequence of a P-PSMA8-101 construct of the disclosure.

FIG. 4C is a schematic diagram of the amino acid sequence of a P-PSMA8-101 construct of the disclosure.

FIG. 5 is a schematic diagram depicting the construction of a CARTyrin of the disclosure and a table contrasting characteristics of Centyrins and antibodies.

FIG. 6A-6E shows the transient expression and function of PSMA CARTyrins. In vitro assays were performed to test the expression and function of the lead PSMA CARTyrins used to produce P-PSMA5-101 and P-PSMA8-101. PSMA CARTyrins were detected on the surface of primary human T cells that were transiently transfected with mRNA encoding the PSMA CARTyrins the night before (FIG. 6A). Briefly, previously activated then frozen Pan T cells were thawed and rested overnight in T cell culture media, cells were electroporated with 10 μg of PSMA CARTyrin mRNA the next evening, and then surface expression analysis was performed the next morning by FACS using soluble recombinant human PSMA protein (rPSMA) for labeling. To test the function of these T cells in vitro, cells were co-cultured with a panel of PSMA-expressing cells for 4 hours and then target cell killing was measured by expression of CD107a, which is a marker for degranulation and a surrogate for T cell killing. Surface expression of PSMA was assessed on K562 cells engineered to stably express PSMA (K562.PSMA) and LNCaP, a human prostate cancer cell line that endogenously expressed PSMA (FIG. 6B). PSMA CARTyrin-expressing T cells were capable of degranulating against all cell lines expressing PSMA (LNCaP, K562.PSMA) with little to no degranulation above background against PSMA− cell lines (K562, PC-3) (FIG. 6C). mRNA encoding PSMA was titrated into a PSMA− cell line, K562, to control for the level of surface expression of PSMA (FIG. 6D). PSMA CARTyrin+ cells exhibited strong cytotoxic function against K562 cells expressing various amounts of surface PSMA (FIG. 6E). These data show that the PSMA CARTyrins can be expressed on the surface of T cells and facilitate cytotoxic function against PSMA+ cell targets.

FIG. 7A-7F shows the phenotype and function of piggyBac manufactured P-PSMA-101. To support in vivo evaluation of the lead PSMA CARTyrins, P-PSMA-101 was constructed using the piggyBac DNA modification system. PSMA CARTyrin was detected on the surface of primary human T cells from a representative donor that were transposed with either P-PSMA5-101 or P-PSMA8-101 plasmids, but not on cells transposed with the P-BCMA-101 plasmid control (FIG. 7A). In both cases, a majority of the CD8+ CAR-T cells were double positive for expression of CD45RA and CD62L, markers commonly associated with a T stem cell memory phenotype (T_{SCM}), following staining and FACS analysis (FIG. 7B). Furthermore, these cells (gated on CD8+ or CD4+) expressed low to no levels of PD-1, Tim-3 and Lag-3 by FACS analysis, which are molecules associated with activation and/or functional T cell exhaustion (FIG. 7C). Next, the effector function of these CAR-T cells was assessed in vitro following co-culture with PSMA-expressing cells. IFN-γ secretion was measured by standard ELISA after 24 hours and detected in the medium when CAR-T cells (from 3 independent donors) were incubated in the presence of their cognate target antigen; P-BCMA-101 secreted IFN-γ only in the presence of K562 cells engineered to express BCMA on the surface (K562.BCMA), whereas P-PSMA-101 secreted IFN-γ only in the presence of cell tumor lines expressing PSMA on the surface (LNCaP and K562.PSMA) (FIG. 7D). In addition, P-PSMA-101 exhibited strong cytotoxic function against LNCaP as measured by a standard killing assay, whereas P-BCMA-101 exhibited little killing capability; data from 2 independent donors (FIG. 7E). The capacity for cell proliferation upon co-culture with a several tumor cell lines was assessed after 96 hours. P-PSMA-101 exhibited a robust capacity for proliferation against PSMA+ LNCaP and 22Rv1 and P-BCMA-101 proliferated against BCMA+H929, whereas neither CAR-T cell proliferated against PSMA-BCMA− cell lines K562 nor DU145 (FIG. 7F). These data show that P-PSMA-101 cells expressed the PSMA CARTyrin on the surface and demonstrated cytotoxic function and proliferative capacity in vitro against PSMA+ cell targets.

FIG. 8A-8F shows the pre-clinical evaluation of P-PSMA8-101 using a murine xenograft model. FIG. 8A is a schematic of the treatment schedule. A murine xenograft model using a luciferase-expressing LNCaP cell line (LNCaP.luc) injected subcutaneously (SC) into NSG mice was utilized to assess in vivo anti-tumor efficacy of P-PSMA8-101. For these in vivo studies, all CAR-T cells were produced using PB delivery of the P-PSMA8-101 plasmid and the Poseida manufacturing process. Mice were injected in the axilla with LNCaP (n=25 to account for poor LNCaP “take” rate) and treated when tumors were established (100-300 mm³ by caliper measurement, 17 days post implantation). Mice were treated with several doses including ultra-low (1×10\hat{ }6), ‘stress’ (5×10\hat{ }6), and standard (10×10\hat{ }6) doses of P-PSMA-101 by IV injection. FIG. 8B is a survival curve graph of anti-tumor activity. FIG. 8C is a bar graph showing the P-PSMA8-101 CD8+ T cell expansion and detection in the blood. FIG. 8D is a series of line graphs showing tumor volume assessment by caliper measurement. FIG. 8E is a line graph showing the bioluminescence of LNCaP tumor by BLI. FIG. 8F shows representative photographs of the bioluminescense of LNCaP tumor in mice quantified in FIG. 8E.

FIG. 9A-9G shows the preclinical evaluation of lead P-PSMA5-101 and P-PSMA8-101 candidates as a “stress” dose using the murine xenograft model. FIG. 9A is a schematic diagram depicting the timeline of a study for the preclinical evaluation of P-PSMA-101 candidates at a ‘stress’ dose using the Murine Xenograft Model. A murine xenograft model using a luciferase-expressing LNCaP cell line (LNCaP.luc) injected subcutaneously (SC) into NSG mice was utilized to assess in vivo anti-tumor efficacy of P-PSMA5-101 and P-PSMA8-101 at a ‘stress’ dose (4×10\hat{ }6) total CAR-T cells. For these in vivo studies, all CAR-T cells were produced using PB delivery of either the P-PSMA5-101 or P-PSMA8-101 plasmid using the Poseida manufacturing process. Mice were injected in the axilla with LNCaP and treated when tumors were established (100-300 mm³ by caliper measurement). Mice were treated with a ‘stress’ dose (4×10\hat{ }6) of P-PSMA-101 by IV injection in order to tease out any possible differences in efficacy between the PSMA5 and the PSMA8 CARs. Anti-tumor activity was evaluated by survival, CD8+ T cell expansion and detection in the blood, tumor volume assessment by caliper measurement, and bioluminescence of LNCaP tumor. P-PSMA5-101 and P-PSMA8-101 at a ‘stress’ dose demonstrated significantly enhanced anti-tumor efficacy and survival in comparison to the T cells (no CAR) control mice against established SC LNCaP.luc solid tumors in NSG mice. Specifically, there was no survival in T cells (no CAR) control animals, 25% survival in the P-BCMA-101 treated group, 75% survival in the P-PSMA5-101 treated group, and 100% survival in animals treated with a ‘stress’ dose of P-PSMA8-101. In the peripheral blood, P-PSMA5-101 and P-PSMA8-101 expanded and gave rise to differentiated effector CARTyrin+ T-cells that were concomitant with a decrease in tumor burden below detectable caliper and bioluminescent imaging limits. These cells then contracted, yet persisted in the peripheral blood. FIG. 9B is a survival curve graph of anti-tumor activity. FIG. 9C is a bar graph showing the P-PSMA5-101 and P-PSMA8-101 CD8+ T cell expansion and detection in the blood. FIG. 9D is a series of line graphs showing tumor volume assessment by caliper measurement. The left panel shows the average of the tumor volume data shown in the series of graphs on the right panel. FIG. 9E is a line graph showing the bioluminescence of LNCaP tumor by BLI. FIG. 9F shows representative photographs of the bioluminescnece of LNCaP tumor mice quantified in FIG. 9E. FIG. 9G is a series of flow cytometry plots showing that P-PSMA-101 (T_{SCM}/T_{CM}) give rise to CARTyrin+ T_{CM}, T_{EM}, and Teff to attack solid tumor. After solid tumor elimination, a population of P-PSMA-101 T_{SCM} persists.

FIG. 10 is a series of flow cytometry plots depicting the abundance of cells moving from an area of live cells (the gated lower right quadrant) to an area populated by apoptotic cells (the upper left quadrant) as a function of increasing dosage of the induction agent (AP1903) in cells modified to express a therapeutic agent (a CARTyrin) alone or in combination with an inducible caspase polypeptide of the disclosure (encoded by an iC9 construct (also known as a “safety switch”) introduced into cells by a piggyBac (PB) transposase) at day 12 post nucleofection.

FIG. 11 is a series of flow cytometry plots depicting the abundance of cells moving from an area of live cells (the gated lower right quadrant) to an area populated by apoptotic cells (the upper left quadrant) as a function of increasing dosage of the induction agent (AP1903) in cells modified to express a therapeutic agent (a CARTyrin) alone or in combination with an inducible caspase polypeptide of the disclosure (encoded by an iC9 construct (also known as a “safety switch”) introduced into cells by a piggyBac (PB) transposase) at day 19 post nucleofection.

FIG. 12 is a pair of graphs depicting a quantification of the aggregated results shown either in FIG. 10 (left graph) or FIG. 11 (right graph). Specifically, these graphs show the impact of the iC9 safety switch on the percent cell viability as a function of the concentration of the induction agent (AP1903) of the iC9 switch for each modified cell type at either day 12 (FIG. 10 and left graph) or day 19 (FIG. 11 and right graph).

FIG. 13 is a bar graph depicting the knock out efficiency of targeting various checkpoint signaling proteins that could be used to armor T-cells. Cas-CLOVER was used to knockout the checkpoint receptors, PD-1, TGFBR2, LAG-3, TIM-3 and CTLA-4 in resting primary human pan T cells. Percent knock-out is shown on the y-axis. Gene editing resulted in 30-70% loss of protein expression at the cell surface as measured by flow cytometry.

FIG. 14 are schematic diagrams of wildtype, null and switch receptors and their effects on intracellular signaling, either inhibitory or stimulatory, in primary T-cells. Binding of the wildtype inhibitory receptor expressed endogenously on a T-cell with its endogenous ligand results in transmission of an inhibitory signal which, in part, reduces T-cell effector function. However, mutation (Mutated null) or deletion (Truncated null) of the intracellular domain (ICD) of a checkpoint receptor protein, such as PD1 (top panel) or TGFBRII (bottom panel), reduces or eliminates its signaling capability when cognate ligand(s) is bound. Thus, expression of engineered mutated or truncated null receptors on the surface of modified T cells results in a competition with endogenously-expressed wildtype receptors for binding of the free endogenous ligand(s), effectively reducing or eliminating delivery of inhibitory signals by endogenously-expressed wildtype receptors. Specifically, any binding by a mutated or null receptor sequesters the endogenous ligand(s) from binding the wildtype receptor and results in dilution of the overall level of checkpoint signaling effectively delivered to the modified T-cell, thereby reducing or blocking checkpoint inhibition and functional exhaustion of the modified T cells. A switch receptor is created by replacement of the wildtype ICD with an ICD from either a co-stimulatory molecule (such as CD3z, CD28, 4-1BB) or a different inhibitory molecule (such as CTLA4, PD1, Lag3). In the former case, binding of the endogenous ligand(s) by the modified switch receptor results in the delivery of a positive signal to the T-cells, thereby helping to enhance stimulation of the modified T cell and potentially enhance target tumor cell killing. In the latter case, binding of the endogenous ligand(s) by the modified switch receptor results in the delivery of a negative signal to the T-cells, thereby eliminating stimulation of the modified T cell and potentially reducing target tumor cell killing. The signal peptide (purple arrow), extracellular domain (ECD) (bright green), transmembrane domain (yellow), intracellular signaling domain (ICD)(orange), and replacement ICD (green) are displayed in the receptor diagrams. “*” indicates a mutated ICD. “+” indicates the presence of a checkpoint signal. “−” indicates the absence of a checkpoint signal.

FIG. 15A is a schematic diagram showing an example of the design of null receptors with specific alterations that may help to increase expression of the receptor on the surface of modified T cells. Examples are shown for PD1 and TGFBRII null receptors and the signal peptide domain (SP), transmembrane domain (TM) and extracellular domain (ECD) of truncated null receptors for PD1 (top panel) and TGFBRII (bottom panel) are displayed. The first of the top four molecules is the wildtype PD-1 receptor, which encodes the wildtype PD-1 SP and TM. For the PD1 null receptor, replacement of PD1 wildtype SP or TM domain (green; light green) with the SP or TM domain of a human T cell CD8a receptor (red) is depicted. The second molecule encodes the CD8a SP along with the native PD-1 TM, the third encodes the wildtype PD-1 SP and the alternative CD8a TM, and the fourth encodes both the alternative CD8a SP and TM. Similarly, for the null receptor of TGFβRII, replacement of the wildtype TGFBRII SP (pink) with a SP domain of a human T cell CD8a receptor (red). The names of the constructs and the amino acid lengths (aa) of each construct protein is listed on the left of the diagram.

FIG. 15B is a series of histograms depicting the expression of the PD1 and TGFBRII null Receptors on the surface of modified primary human T cells as determined by flow cytometry. Each of the six truncated null constructs from FIG. 15A were expressed on the surface of primary human T cells. T cells were stained with either anti-PD1 (top; blue histograms) or anti-TGFβRII (bottom; blue histograms), or isotype control or secondary only (gray histograms). Cells staining positive for PD-1 or TGFβRII expression were gated (frequency shown above gate) and mean fluorescence intensity (MFI) value is displayed above each positive histogram. The names of the null receptor constructs are depicted above each plot. Both null receptor gene strategies, replacement of the wildtype SP with the alternative CD8a were successfully expressed. 02.8a SP-PD-1 and 02.8aSP-TGFβRII resulted in the highest level of expression at the T-cell surface. 02.8aSP-PD-1 null receptor exhibited an MFI of 43,680, which is 177-fold higher than endogenous T cell PD-1 expression and 2.8-fold higher than the wildtype PD-1 null receptor. 02.8aSP-TGFβRII null receptor exhibited an MFI of 13,809, which is 102-fold higher than endogenous T cell TGFβRII expression and 1.8-fold higher than the wildtype TGFβRII null receptor. Replacement of wildtype SP with the alternative CD8α SP for both PD1 and TGRBRII results in enhanced surface expression of the null or Switch receptor, which may help to maximize reduction or blockage of checkpoint inhibition upon binding and sequestration of the endogenous ligand(s).

FIG. 16A-B is a pair of schematic diagrams depicting NF-KB inducible vectors for expression in T-cells. Two T cell activation NF-KB inducible vectors were developed; one with the gene expression system (GES) in the forward orientation (A) and the other in the complementary direction (B), both preceding the constitutive EF1a promoter. These vectors also direct expression of a CAR molecule and a DHFR selection gene, separated by a T2A sequence. Both the conditional NF-KB inducible system and the EF1a directed genes are a part of a piggyBac transposon which can be permanently integrated into T cells using electroporation (EP). Once integrated into the genome, the T cells will constitutively express the CAR on the membrane surface and the DHFR within the cell, while expression of the NF-KB inducible gene, GFP, will be expressed to the highest level only upon T cell activation.

FIG. 17 is a pair of graphs depicting NF-KB inducible expression of GFP in activated T cells. T cells were nucleofected with a piggyBac vector expressing an anti-BCMA CAR and a DHFR mutein gene under control of an EF1a promoter along with the absence (No GES control) or presence of an NF-KB inducible expression system driving GFP expression in either the forward (pNFKB-GFP forward) or reverse orientation (pNFKB-GFP reverse). Cells were cultured in the presence of methotrexate selection until the cells were almost completely resting (Day 19) and GFP expression was assessed at Day 5 and Day 19. At Day 5, all T cells are proliferating and highly stimulated, with cells harboring the NF-KB inducible expression cassette producing high levels of GFP due to strong NFκB activity. The No GES control cells did not express detectable levels of GFP. By Day 19, the GES T cells were almost fully resting and GFP expression was significantly lower than Day 5 (˜⅛ MFI), since NFκB activity is lower. GFP expression is still observed at Day 19, which may due to the long half-life of GFP protein (˜30 hr), or, basal level of NFκB activity through, for example, a TCR, a CAR, a cytokine receptor, or a growth factor receptor signal.

FIG. 18 is a series of graphs depicting anti-BCMA CAR-mediated activation of NF-KB inducible expression of GFP in presence of BCMA+ tumor cells. T cells were either unmodified (Mock T cells) or nucleofected with a piggyBac vector expressing an anti-BCMA CAR and a DHFR mutein gene under control of an EF1a promoter along with the absence (No GES control) or presence of an NF-KB inducible expression system driving GFP expression in either the forward (pNFKB-GFP forward) or reverse orientation (pNFKB-GFP reverse). All cells were cultured for 22 days, either with or without methotrexate selection (Mock T cells), until the cells were almost completely resting. Cells were then stimulated for 3 days in the absence (No stimulation) or presence of BCMA− (K562), BMCA+(RPMI 8226), or positive control anti-CD3 anti-CD28 activation reagent (CD3/28 stimulation). GFP expression was undetectable under all conditions with the No GES control or Mock T cells. However, while pNFKB-GFP forward- and reverse-transposed cells exhibited little GFP expression over the No stimulation control when cultured with BCMA− K562 cells, they both demonstrated dramatic upregulation of gene expression either in the presence of BCMA+ tumor cells or under positive control conditions. Little difference in GFP expression was observed between the pNFKB-GFP forward- and reverse-transposed cells that were cocultured with BCMA+ tumor cells.

FIG. 19 is a series of graphs demonstrating that the Expression level of inducible gene can be regulated by number of response elements preceding the promoter T cells were nucleofected with a piggyBac vector encoding an anti-BCMA CARTyrin followed by a selection gene, both under control of a human EF1a promoter. Further, vectors either additionally encoded the conditional NF-KB inducible gene expression system driving expression of a truncated CD19 protein (dCD19) and included a number of NFκB response elements (RE) varying from 0-5, no GES (No GES), or received an electroporation pulse but no piggyBac nucleic acid (Mock). Data are shown for only the GES in the reverse (opposite) direction/orientation. All cells were cultured for 18 days and included selection for piggyBac-modified T cells using methotrexate addition. Cells were then stimulated for 3 days using anti-CD3 anti-CD28 bead activation reagent and dCD19 surface expression was assessed by FACS at Days 0, 3 and 18, and data are shown as FACS histograms and MFI of target protein staining. The x-axes of each of the FACS histograms are depicted in logarithmic scale (0, 10³, 10⁴, 10⁵). The samples plotted each of the FACS histograms are NFKB-A08-DHFR_Rev002.fcs, NFKB-A08_DHFR_Rev-RE4X_12.fcs, NFKB-A08_DHFR_Rev-RE3X_011.fcs, NFKB-A08_DHFR_Rev-RE2X_010.fcs, NFKB-A08_DHFR_Rev-RE1X_009.fcs, NFKB-A08_DHFR_Rev-RE0X_008.fcs, NFKB-A08_DHFR_v5_013.fcs and NFKB-A08_DHFR_MOCK_014.fcs, from top to bottom. Surface dCD19 expression was detected at low levels at Day 0 in all T cells transposed with vectors encoding the GES. At 3 days post-stimulation, dramatic upregulation of dCD19 expression was observed for all T cells expressing the GES, with a greater fold increase in surface expression in those with higher numbers of REs. Thus, surface dCD19 expression was directly proportional with the number of REs encoded in the GES. No dCD19 was detected on the surface of T cells that did not harbor the GES: No GES and Mock controls.

FIG. 20 is a schematic depiction of the Csy4-T2A-Clo051-G4Slinker-dCas9 construct map (Embodiment 2).

FIG. 21 is a schematic depiction of the pRT1-Clo051-dCas9 Double NLS construct map (Embodiment 1).

FIG. 22 is a schematic diagram depicting the timeline of a study for the preclinical evaluation of lead P-PSMA-101 candidates in a prostate cancer bone metastasis Murine Xenograft Model. A murine xenograft model using a luciferase-expressing PC3 cell line engineered to express human PSMA protein (PC3.lucGFP.hPSMA) was injected peri-tibially (IT) into NSG mice and was utilized to assess in vivo anti-tumor efficacy of P-PSMA5-101 and P-PSMA8-101 at two different doses—a ‘stress’ dose of 4×10\hat{ }6 and standard dose of 12×10\hat{ }6 total CAR-T cells. As negative controls, T cells alone (not expressing a CAR; 12e6 dose) or P-BCMA-101 T cells (expressing an irrelevant anti-BCMA CAR; 12e6 dose) were also included. For these in vivo studies, all CAR-T cells were produced using PB delivery of either the P-PSMA5-101 or P-PSMA8-101 plasmid using the Poseida manufacturing process. Mice were injected peri-tibially with Pc3.lucGFP.hPSMA and treated with CAR-T cells four days later (all tumor-challenged mice had detectable tumors by bioluminescent imaging (BLI) ranging from 1-5×10\hat{ }6 in total luminescent flux (p/sec/m²)).

FIG. 23 is a graph showing dose efficacy of P-PSMA5-101 and P-PSMA8-101 at either a ‘stress’ dose or standard dose demonstrated anti-tumor efficacy in comparison to the T cells (no CAR) alone or P-BCMA-101 treated control mice against established IT PC3.lucGFP.hPSMA solid tumors in NSG mice.

FIG. 24 is a schematic diagram depicting a piggyBac P-PSMA-101 nanotransposon of the disclosure.

FIG. 25 is a graph showing electroporation (EP) delivery of either P-PSMA-101 piggyBac plasmid (light gray bars) or P-PSMA-101 piggyBac nanotransposon (dark gray bars) in combination with super piggyBac transposase enzyme resulted in high transposition efficiency in human pan T cells (5 days post-EP) as measured by surface expression of PSMA CARTyrin (Percent Transposition (%)).

FIG. 26 is a series of graphs showing that human CAR-T cells produced using anti-PSMA CAR nanotransposons (NT) were capable of killing target tumor cells. Anti-PSMA CAR T cells produced using either full-sized piggyBac plasmids (FP) or piggyBac nanotransposon (NT) were produced using the standard Poseida process. Killing of K562 cells engineered to express PSMA (K562.PSMA) by CAR-T cells at the indicated effector to target ratios. These data show that all CAR-T cells, whether produced using FP or NT, were capable of killing target tumor cells in an antigen-dependent manner. This was true for CAR-T cells that were produced from human pan T cells from two different normal donors.

FIG. 27 is a series of graphs showing that human CAR-T cells produced using anti-PSMA CAR nanotransposons (NT) were comparable in phenotypic composition. Anti-PSMA CAR T cells produced using either full-sized piggyBac plasmids (FP) or piggyBac nanotransposon (NT) were manufactured using the standard Poseida process. Phenotypic analysis of memory T cell markers and activation/exhaustion markers (data not shown) was performed. These data show that all CAR-T cells, whether produced using FP or NT, exhibited a similar phenotypic composition of CD45RA+CD62L+ (Tscm), CD45RA− CD62L+ (Tcm), CD45RA−CD62L− (Tem), and CD45RA+CD62L− (Teff) cells. In addition, comparable levels of expression of CCR7 (CD197), CD127, CD27, LAGS, TIM3, CXCR3, PD-1, and CD25 was observed (data not shown). This was true for CAR-T cells that were produced from human pan T cells from two different normal donors.

FIG. 28 is a series of graphs showing that human CAR-T cells produced using anti-PSMA CAR nanotransposons (NT) have similar integrated copy number. Anti-PSMA CAR T cells produced using either full-sized piggyBac plasmids (FP) or piggyBac nanotransposon (NT) were manufactured using the standard Poseida process. Average copy number of integrated transposons was measured by quantitative PCR. These data show that in two different donors, all CAR-T cells, whether produced using FP or NT, exhibited a similar integrated copy number of transposons.

FIG. 29A is a schematic diagram showing preclinical evaluation of the P-PSMA-101 transposon when delivered by a full-length plasmid (FLP) versus a nanotransposon (NT) at ‘stress’ doses using the murine xenograft model. The murine xenograft model using a luciferase-expressing LNCaP cell line (LNCaP.luc) injected subcutaneously (SC) into NSG mice was utilized to assess in vivo anti-tumor efficacy of the P-PSMA-101 transposon as delivered by a full-length plasmid (FLP) or a nanotransposon (NT) at two different ‘stress’ doses (2.5×10\hat{ }6 or 4×10\hat{ }6) of total CAR-T cells from two different normal donors. All CAR-T cells were produced using piggyBac (PB) delivery of P-PSMA-101 transposon using either FLP or NT delivery. Mice were injected in the axilla with LNCaP and treated when tumors were established (100-200 mm³ by caliper measurement). Mice were treated with two different ‘stress’ doses (2.5×10\hat{ }6 or 4×10\hat{ }6) of P-PSMA-101 CAR-Ts by IV injection for greater resolution in detecting possible functional differences in efficacy between transposon delivery by the FLP and the NT.

FIG. 29B are a series of graphs showing the tumor volume assessment of mice treated as described in FIG. 29A. Tumor volume assessment by caliper measurement for control mice (black), Donor #1 FLP mice (red), Donor #1 NT mice (blue), Donor #2 FLP mice (orange), and Donor #2 NT mice (green) as displayed as group averages with error bars (top) and individual mice (bottom). The y-axis shows the tumor volume (mm³) assessed by caliper measurement. The x-axis shows the number of days post T cell treatment. Delivered by NT, P-PSMA-101 transposon at a ‘stress’ dose demonstrated enhanced anti-tumor efficacy as measured by caliper in comparison to the FLP and control mice against established SC LNCaP.luc solid tumors.

FIG. 30 is a schematic diagram depicting a T-cell receptor (TCR) and co-receptors CD28 and CD2.

FIG. 31 is a schematic diagram depicting primary and secondary co-stimulation is delivered to T-cell via binding of agonist mAbs (anti-CD3, anti-CD28, and anti-CD2). Full T-cell activation critically depends on TCR engagement in conjunction with a second signal by co-stimulatory receptors that boost the immune response. Primary and secondary co-stimulation can be delivered to T-cell via treatment and engagement of surface receptors with reagents displaying agonist mAbs (E.g. anti-CD3, anti-CD28, anti-CD2, beads conjugated with these mAbs, multimeric complexes of these mAbs, etc. . . . ).

FIG. 32 is a schematic diagram showing that, in absence of TCR, stimulation is enhanced with expression of Chimeric Stimulatory Receptors (CSRs). In the presence of surface-expressed CSR/s, either transiently or stably expressed, enhanced primary and secondary co-stimulatory signals are delivered when T cell is treated with reagents displaying agonist mAbs. Since a fuller T-cell activation is achieved via CSR-mediated stimulatory signals, T cell activation and expansion is enhanced.

FIG. 33A is a schematic diagram depicting an exemplary CSR CD28z of the disclosure.

FIG. 33B is an amino sequence encoding the CSR CD28z shown in FIG. 33A.

FIG. 33C is a nucleotide sequence encoding the CSR CD28z shown in FIG. 33A.

FIG. 34A is a schematic diagram depicting an exemplary CSR CD2z of the disclosure.

FIG. 34B is an amino sequence encoding the CSR CD2z shown in FIG. 34A.

FIG. 34C is a nucleotide sequence encoding the CSR CD2z shown in FIG. 34A.

FIG. 35 is a series of graphs showing that CSRs are expressed on the surface of T cells and do not lead to cellular activation in the absence of exogenous stimulation. Pan T cells from normal blood donors were stimulated with anti-CD3/anti-CD28 beads in standard T cell culture media, then rested. These cells were then electroporated (BTX ECM 830 electroporator @ 500V for 700 μs) with 10 μg of mRNA encoding either CD28 CSR, CD2 CSR, or wild-type CD19 control. Two days later the electroporated cells were examined by flow cytometry for surface-expression of each molecule and data are shown as stacked histograms. In addition, cell size (FSC-A) and CD69 expression was evaluated as a possible indication of cellular activation above the Mock electroporated control cells. Increased surface expression of CD28, CD2, and CD19 were detected in T cells electroporated either with CD28z CSR, CD2z CSR or CD19, respectively. Expression of these molecules on the surface of T cells did not intrinsically activate the cells in the absence of exogenous stimulation.

FIG. 36 is a graph showing that delivery of CSR enhances the expansion of CAR-T cells. CSRs were delivered to CAR-T cells either transiently by mRNA or stably by piggyBac™. Pan T cells isolated from the blood of a normal donor were genetically modified using the piggyBac™ DNA modification system and the standard Poseida process. Cells were co-electroporated in a single reaction with mRNA encoding the Super piggyBac transposase enzyme (SPB), a transposon encoding a BCMA CAR and selection gene, along with an additional mRNA encoding a CSR (either CD28z or CD2z; resulting in transient expression) or a CD19 mRNA control, or, with a transposon encoding a BCMA CAR, selection gene and a CSR (either CD28z or CD2z; resulting in stable expression). The cells were subsequently stimulated with agonist mAbs anti-CD2, anti-CD3 and anti-CD28, and were later selected for genetic modification over the course of a 19 day culture period. At the end of the initial culture period all T cells expressed the CAR, indicating successful selection for genetically-modified cells (data not shown). Bars represent total live CAR-T cells in well and numbers indicate fold-enhancement of expansion above CAR-T cells produced in the absence of a CSR or a CD19 mRNA control. In the samples expressing either CD2z or CD28z CSR, either transiently or stably, a greater degree of expansion of the CAR-T cells.

FIG. 37 is a series of bar graphs showing that expression of CSRs does not significantly affect CAR-T cell cytotoxicity. CSRs were delivered to CAR-T cells either transiently by mRNA or stably by piggyBac™. Pan T cells isolated from the blood of a normal donor were genetically modified using the piggyBac™ DNA modification system and the standard Poseida process. Cells were co-electroporated in a single reaction with mRNA encoding the Super piggyBac transposase enzyme (SPB), a transposon encoding a BCMA CAR and selection gene, along with an additional mRNA encoding a CSR (either CD28z or CD2z; resulting in transient expression), or, with a transposon encoding a BCMA CAR, selection gene and a CSR (either CD28z or CD2z; resulting in stable expression). The cells were subsequently stimulated with agonist mAbs anti-CD2, anti-CD3 and anti-CD28, and were later selected for genetic modification over the course of a 19 day culture period. At the end of the initial culture period all T cells expressed the CAR, indicating successful selection for genetically-modified cells (data not shown). To assess CAR-T cell ability to kill, cells were co-cultured with engineered K562-BCMA-Luciferase (eK562-Luc.BCMA) or negative control line K562-Luciferase (eK562-Luc) for 48 hours at 10:1, 3:1, or 1:1 E:T ratios. Luciferase signal was measured to determine cytotoxicity. Killing of eK562-Luc is shown in bar graph on left, while killing of eK562-Luc.BCMA is shown in bar graph on right. All CAR⁺ T cells expressed an anti-BCMA specific CAR and exhibited similar in vitro cytotoxicity against BCMA+ target cells. In summary, this activity was not significantly affected by transient or stable CSR co-expression.

### DETAILED DESCRIPTION

The disclosure provides chimeric antigen receptors (CARs) comprising at least one Centyrin (CARTyrin). Chimeric antigen receptors of the disclosure may comprise more than one Centyrin. For example, a bi-specific CARTyrin may comprise two Centyrins that specifically bind two distinct antigens. In preferred embodiments, CARTyrins of the disclosure comprise at least one Centryrin that specifically binds a sequence of PSMA, and, therefore, are referred to as PSMA-specific Centryins. CARTyrins of the disclosure that comprise at least one PSMA-specific Centryrin are referred to herein as anti-PSMA CARTyrins for specifically binding a sequence of PSMA.

Centyrins of the disclosure specifically bind to an antigen. Chimeric antigen receptors of the disclosure comprising one or more Centyrins that specifically bind an antigen may be used to direct the specificity of a cell, (e.g. a cytotoxic immune cell) towards the specific antigen.

Centyrins of the disclosure may comprise a consensus sequence comprising

(SEQ ID NO: 18018)
LPAPKNLVVSEVTEDSLRLSWTAPDAAFDSFLIQYQESEKVGEAINLTVP
GSERSYDLTGLKPGTEYTVSIYGVKGGHRSNPLSAEFTT.

Chimeric antigen receptors of the disclosure may comprise a signal peptide of human CD2, CD3δ, CD3ε, CD3γ, CD3ζ, CD4, CD8α, CD19, CD28, 4-1BBor GM-CSFR. A hinge/spacer domain of the disclosure may comprise a hinge/spacer/stalk of human CD8α, IgG4, and/or CD4. An intracellular domain or endodomain of the disclosure may comprise an intracellular signaling domain of human CD3ζ and may further comprise human 4-1BB, CD28, CD40, ICOS, MyD88, OX-40 intracellular segment, or any combination thereof. Exemplary transmembrane domains include, but are not limited to a human CD2, CD3δ, CD3ε, CD3γ, CD3ζ, CD4, CD8α, CD19, CD28, 4-1BBor GM-CSFR transmembrane domain.

The disclosure provides genetically modified cells, such as T cells, NK cells, hematopoietic progenitor cells, peripheral blood (PB) derived T cells (including T cells from G-CSF-mobilized peripheral blood), umbilical cord blood (UCB) derived T cells rendered specific for one or more antigens by introducing to these cells a CARTyrin of the disclosure. Cells of the disclosure may be modified by electrotransfer of a transposon encoding a CARTyrin of the disclosure and a plasmid comprising a sequence encoding a transposase of the disclosure (preferably, the sequence encoding a transposase of the disclosure is an mRNA sequence).

### Further Modifications of Cell Compositions

The disclosure provides a chimeric stimulatory receptor (CSR) comprising: (a) an ectodomain comprising an activation component and (b) a transmembrane domain or a cell-membrane attachment region, wherein the combination of (a) and (b) is non-naturally occurring. In some embodiments, this CSR binds a component of the environment and changes the cellular consequence of that signaling by competing with full-length or transmembrane versions of the receptor to reduce the intracellular signal resulting from binding of the component of the environment to the activation component.

The disclosure provides a chimeric stimulatory receptor (CSR) comprising: (a) an ectodomain comprising a activation component; (b) a transmembrane domain; and (c) an endodomain comprising at least one signal transduction domain; wherein the combination of (a), (b) and (c) is non-naturally occurring.

In some embodiments of the CSRs of the disclosure, the activation component of (a) is isolated or derived from a first protein. In some embodiments, the signal transduction domain of (c) is isolated or derived from a second protein. In some embodiments, the first protein and the second protein are not identical.

In some embodiments of the CSRs of the disclosure, the CSR is a switch receptor that translates binding of the activation component extracellularly with either suppressing a signal or transducing a qualitatively different signal than would be transduced by a wild type, full-length or transmembrane version of the first protein. Because a CSR switch receptor is chimeric with respect to the extracellular and intracellular domains, the CSR can switch the consequence of binding the extracellular activation component from the naturally-occurring scenario to an engineered, non-naturally occurring, scenario.

In some embodiments of the CSRs of the disclosure, the activation component comprises one or more of a component of a human transmembrane receptor, a human cell-surface receptor, a T-cell Receptor (TCR), a component of a TCR complex, a component of a TCR co-receptor, a component of a TCR co-stimulatory protein, a component of a TCR inhibitory protein, a cytokine receptor, and a chemokine receptor. In some embodiments, the Activation component comprises a portion of one or more of a component of a human transmembrane receptor, a human cell-surface receptor, a T-cell Receptor (TCR), a component of a TCR complex, a component of a TCR co-receptor, a component of a TCR co-stimulatory protein, a component of a TCR inhibitory protein, a cytokine receptor, and a chemokine receptor to which an agonist of the activation component binds.

In some embodiments of the CSRs of the disclosure, the agonist comprises one or more of a small organic or inorganic molecule, a nucleic acid, an amino acid, an antibody or a fragment thereof, an antibody mimetic, an aptamer, a scaffold protein, a ligand, a receptor, a naturally occurring biomolecule, and a non-naturally occurring molecule (organic or inorganic).

In some embodiments of the CSRs of the disclosure, the Activation component comprises a CD2 protein or a portion thereof to which an agonist binds.

In some embodiments of the CSRs of the disclosure, the signal transduction domain comprises one or more of a component of human signal transduction domain, a T-cell Receptor (TCR), a component of a TCR complex, a component of a TCR co-receptor, a component of a TCR co-stimulatory protein, a component of a TCR inhibitory protein, a cytokine receptor, and a chemokine receptor. In some embodiments, the signal transduction domain comprises a CD3 protein. In some embodiments, the CD3 protein comprises a CD3ζ protein.

In some embodiments of the CSRs of the disclosure, the endodomain further comprises a cytoplasmic domain. In some embodiments, the sequence encoding the cytoplasmic domain comprises a sequence encoding a co-stimulatory protein. In some embodiments, the cytoplasmic domain is isolated or derived from a third protein. In some embodiments, the first protein and the third protein are identical.

In some embodiments of the CSRs of the disclosure, the ectodomain further comprises a signal peptide. In some embodiments, the signal peptide is derived from a fourth protein. In some embodiments, the first protein and the fourth protein are identical.

In some embodiments of the CSRs of the disclosure, the transmembrane domain is isolated or derived from a fifth protein. In some embodiments, the first protein and the fifth protein are identical.

In some embodiments of the CSRs of the disclosure, the CSR comprises an ectodomain comprising a signal peptide having a sequence isolated or derived from a CD2 protein and a Activation component comprising a sequence isolated or derived from a CD2 protein or a portion thereof to which an agonist binds, a transmembrane domain comprising a sequence isolated or derived from a CD2 protein, and an endodomain comprising a cytoplasmic domain comprising a sequence isolated or derived from a CD2 protein and a signal transduction domain comprising a sequence isolated or derived form a CD3ζ protein.

In some embodiments of the CSRs of the disclosure, the Activation component does not bind a naturally-occurring molecule.

In some embodiments of the CSRs of the disclosure, the CSR does not transduce a signal upon binding of the Activation component to a naturally-occurring molecule. In some embodiments, the ectodomain comprises a modification. In some embodiments, the modification comprises a mutation or a truncation of a sequence encoding the Activation component when compared to a wild type sequence of the first protein.

In some embodiments of the CSRs of the disclosure, the Activation component binds to a non-naturally occurring molecule.

In some embodiments of the CSRs of the disclosure, the CSR selectively transduces a signal upon binding of the Activation component to a non-naturally occurring molecule.

The disclosure provides a nucleic acid sequence encoding the CSR of the disclosure.

The disclosure provides a vector comprising the nucleic acid sequence encoding the CSR of the disclosure.

The disclosure provides a vector comprising the nucleic acid sequence encoding the CSR of the disclosure.

The disclosure provides a transposon comprising the nucleic acid sequence encoding the CSR of the disclosure.

The disclosure provides a cell comprising the CSR of the disclosure.

The disclosure provides a cell comprising the nucleic acid encoding the CSR of the disclosure.

The disclosure provides a cell comprising the vector comprising the nucleic acid sequence encoding the CSR of the disclosure.

The disclosure provides a cell comprising the transposon comprising the nucleic acid sequence encoding the CSR of the disclosure.

The disclosure provides a composition comprising the CSR of the disclosure.

The disclosure provides a composition comprising the nucleic acid encoding the CSR of the disclosure.

The disclosure provides a composition comprising the vector comprising the nucleic acid sequence encoding the CSR of the disclosure.

The disclosure provides a composition comprising the transposon comprising the nucleic acid sequence encoding the CSR of the disclosure.

The disclosure provides a composition comprising a cell of the disclosure, including those comprising a sequence encoding a CSR and/or expressing a CSR of the disclosure. The disclosure provides a composition comprising a plurality of cells of the disclosure, including those comprising a sequence encoding a CSR and/or expressing a CSR of the disclosure.

The disclosure provides a modified cell comprising: (a) a sequence encoding a CSR of the disclosure and (b) a sequence encoding an inducible proapoptotic polypeptide; and wherein the cell is a T-cell.

The disclosure provides a modified cell comprising: (a) a sequence encoding a CSR of the disclosure and (b) a sequence encoding an inducible proapoptotic polypeptide. In some embodiments of the modified cells of the disclosure, the modified cells further comprise a sequence encoding a non-naturally occurring antigen receptor, and/or a sequence encoding a therapeutic polypeptide.

In some embodiments of the modified cells of the disclosure, including those wherein the modified cell comprises a sequence encoding a non-naturally occurring antigen receptor, the non-naturally occurring antigen receptor comprises a chimeric antigen receptor (CAR). In some embodiments, the CAR comprises: (a) an ectodomain comprising an antigen recognition region, (b) a transmembrane domain, and (c) an endodomain comprising at least one costimulatory domain. In some embodiments, the ectodomain of (a) of the CAR further comprises a signal peptide. In some embodiments, the ectodomain of (a) of the CAR further comprises a hinge between the antigen recognition region and the transmembrane domain. In some embodiments, the endodomain comprises a human CD3ζ endodomain. In some embodiments, the at least one costimulatory domain comprises a human 4-1BB, CD28, CD40, ICOS, MyD88, OX-40 intracellular segment, or any combination thereof. In some embodiments, the at least one costimulatory domain comprises a human CD28 and/or a 4-1BB costimulatory domain.

In some embodiments of the modified cells of the disclosure, a transposon, a vector, a donor sequence or a donor plasmid comprises the sequence encoding the CSR and/or the sequence encoding the inducible proapoptotic polypeptide. In some embodiments, the transposon, the vector, the donor sequence or the donor plasmid further comprises a sequence encoding a non-naturally occurring antigen receptor or a sequence encoding a therapeutic protein. In some embodiments, the transposon, the vector, the donor sequence, or the donor plasmid further comprises the sequence encoding the selection marker. In some embodiments, the transposon is a piggyBac or a piggy-Bac like transposon.

In some embodiments of the modified cells of the disclosure, the sequence encoding the CSR is transiently expressed in the cell and wherein the sequence encoding the inducible proapoptotic polypeptide is stably expressed in the cell. In some embodiments, a sequence encoding a non-naturally occurring antigen receptor or a sequence encoding a therapeutic protein is stably expressed in the cell. In some embodiments, a first transposon, a first vector, a first donor sequence, or a first donor plasmid comprises the sequence encoding the CSR. In some embodiments, a second transposon, a second vector, a second donor sequence, or a second donor plasmid comprises one or more of the sequence encoding the inducible proapoptotic polypeptide, the sequence encoding a non-naturally occurring antigen receptor, the sequence encoding a therapeutic protein. In some embodiments, the first transposon, the first vector, the first donor sequence, or the first donor plasmid further comprises a sequence encoding a first selection marker. In some embodiments, the second transposon, the second vector, the second donor sequence, or the second donor plasmid further comprises a sequence encoding a second selection marker. In some embodiments, the first selection marker and the second selection marker are not identical.

In some embodiments of the modified cells of the disclosure, the selection marker is a cell surface marker. In some embodiments, the cell surface marker distinguishes cells when sorted by the marker or a detectable tag. In some embodiments, the detectable tag is fluorescent or magnetic.

In some embodiments of the modified cells of the disclosure, the selection marker comprises a protein that is active in dividing cells and not active in non-dividing cells. In some embodiments, the selection marker comprises a metabolic marker. In some embodiments, the selection marker comprises a dihydrofolate reductase (DHFR) mutein enzyme. In some embodiments, the DHFR mutein enzyme comprises or consists of the amino acid sequence of:

```
(SEQ ID NO: 17012)
    1 MVGSLNCIVA VSQNMGIGKN GDFPWPPLRN ESRYFQRMTT TSSVEGKQNL
  61 VIMGKKTWFS IPEKNRPLKG RINLVLSREL KEPPQGAHFL SRSLDDALKL
121 TEQPELANKV DMVWIVGGSS VYKEAMNHPG HLKLFVTRIM QDFESDTFFP
181 EIDLEKYKLL PEYPGVLSDV QEEKGIKYKF EVYEKND.
```

In some embodiments, the amino acid sequence of the DHFR mutein enzyme further comprises a mutation at one or more of positions 80, 113, or 153. In some embodiments, the amino acid sequence of the DHFR mutein enzyme comprises one or more of a substitution of a Phenylalanine (F) or a Leucine (L) at position 80, a substitution of a Leucine (L) or a Valine (V) at position 113, and a substitution of a Valine (V) or an Aspartic Acid (D) at position 153.

### Chimeric Stimulatory Receptors (CSRs)

The disclosure provides a Chimeric Stimulatory Receptor (CSR) to deliver CD3z primary stimulation to T cells (and, consequently, an endogenous CD3ζ) when stimulated using standard activation/stimulation reagents, including agonist anti-CD3 mAb.

Chimeric Stimulatory Receptors (CSRs) of the disclosure provide a CD3ζ stimulus to enhance activation and expansion of T cells. In some embodiments, CSRs of the disclosure comprise an agonist mAb epitope extracellularly and a CD3ζ stimulatory domain intracellularly and, functionally, convert an anti-CD28 or anti-CD2 binding event on the surface into a CD3z signaling event in an allogeneic T cell modified to express the CSR. In some embodiments, a CSR comprises a wild type CD28 or CD2 protein and a CD3z intracellular stimulation domain, to produce CD28z CSR and CD2z CSR, respectively. In preferred embodiments, CD28z CSR and/or CD2z CSR further express a non-naturally occurring antigen receptor and/or a therapeutic protein. In preferred embodiments, the non-naturally occurring antigen receptor comprises a Chimeric Antigen Receptor.

In certain embodiments, the CD28z CSR is encoded by an amino acid sequence comprising

(SEQ ID NO: 17910)
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRSRVKFSRSADAPAYKQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPR.

In certain embodiments, the CD28z CSR is encoded by a nucleic acid sequence comprising

(SEQ ID NO: 17911)
atgctgagactgctgctggccctgaatctgttccccagcatccaagtgac
cggcaacaagatcctggtcaagcagagccctatgctggtggcctacgaca
acgccgtgaacctgagctgcaagtacagctacaacctgttcagcagagag
ttccgggccagcctgcacaaaggactggattctgctgtggaagtgtgcgt
ggtgtacggcaactacagccagcagctgcaggtctacagcaagaccggct
tcaactgcgacggcaagctgggcaatgagagcgtgaccttctacctgcaa
aacctgtacgtgaaccagaccgacatctatttctgcaagatcgaagtgat
gtacccgcctccttacctggacaacgagaagtccaacggcaccatcatcc
acgtgaagggcaagcacctgtgtccttctccactgttccccggacctagc
aagcctttctgggtgctcgttgttgttggcggcgtgctggcctgttatag
cctgctggttacagtggccttcatcatcttttgggtccgaagcaagcgga
gccggctgctgcacagcgactacatgaacatgacccctagacggcccgga
ccaaccagaaagcactaccagccttacgctcctcctagagacttcgccgc
ctaccggtccagagtgaagttctccagatccgccgatgctcccgcctata
agcagggccagaaccagctgtacaacgagctgaacctggggagaagagaa
gagtacgatgtgctggacaagcggagaggcagagatcctgagatgggcgg
caagcccagacggaagaatcctcaagagggcctgtacaatgaactgcaga
aagacaagatggccgaggcctacagcgagatcggaatgaagggcgagcgc
agaagaggcaagggacacgatggactgtaccagggcctgagcaccgccac
caaggatacctatgatgccctgcacatgcaggccctgcctccaaga.

In certain embodiments, the CD2z CSR is encoded by an amino acid sequence comprising

(SEQ ID NO: 17912)
MSFPCKFVASFLLIFNVSSKGAVSKEITNALETWGALGQDINLDIPSFQM
SDDIDDIKWEKTSDKKKIAQFRKEKETFKEKDTYKLFKNGTLKIKHLKTD
DQDIVKVSIVDTKGKNVLEKIFDLKIQERVSKPKISWTCINTTLTCEVMN
GTDPELNLYQDGKHLKLSQRVITHKWTTSLSAKFKCTAGNKVSKESSVEP
VSCPEKGLDIVLIIGICGGGSLLMVFVALLVFYITKRKKQRSRRNDEELE
TRAHRVATEERGRKPHQIPASTPQNPATSQHPPPPPGHRSQAPSHRPPPP
GHRVQHQPQKRPPAPSGTQVHQQKGPPLPRPRVQPKPPHGAAENSLSPSS
NRVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR

In certain embodiments, the CD2z CSR is encoded by a nucleic acid sequence comprising

(SEQ ID NO: 17913)
atgagcttcccttgcaagttcgtggccagcttcctgctgatcttcaacgt
gtcctctaagggcgccgtgtccaaagagatcacaaacgccctggaaacct
ggggagccctcggccaggatattaacctggacatccccagcttccagatg
agcgacgacatcgatgacatcaagtgggagaaaaccagcgacaagaagaa
gatcgcccagttccggaaagagaaagagacattcaaagagaaggacacct
acaagctgttcaagaacggcaccctgaagatcaagcacctgaaaaccgac
gaccaggacatctataaggtgtccatctacgacaccaagggcaagaacgt
gctggaaaagatcttcgacctcaagatccaagagcgggtgtccaagccta
agatcagctggacctgcatcaacaccacactgacctgcgaagtgatgaac
ggcacagaccccgagctgaacctgtaccaggatggcaaacacctgaagct
gagccagcgcgtgatcacccacaagtggacaacaagcctgagcgccaagt
tcaagtgcaccgccggaaacaaagtgtctaaagagtccagcgtcgagccc
gtgtcttgccctgaaaaaggactggacatctacctgatcatcggcatctg
tggcggcggaagcctgctgatggtgtttgtggctctgctggtgttctaca
tcaccaagcggaagaagcagcggagcagacggaacgacgaggaactggaa
acacgggcccatagagtggccaccgaggaaagaggcagaaagccccacca
gattccagccagcacaccccagaatcctgccacctctcaacaccctccac
ctccacctggacacagatctcaggccccatctcacagacctccaccacct
ggtcatcgggtgcagcaccagcctcagaaaagacctcctgctcctagcgg
cacacaggtgcaccagcaaaaaggacctccactgcctcggcctagagtgc
agcctaaacctcctcatggcgccgctgagaacagcctgtctccaagcagc
aacagagtgaagttcagccgcagcgccgatgctcctgcctataagcaggg
acagaaccagctgtacaacgagctgaatctggggcgcagagaagagtacg
atgtgctggacaagcggagaggcagagatcctgagatgggcggcaagccc
agacggaagaatcctcaagagggcctgtataatgagctgcagaaagacaa
gatggccgaggcctacagcgagatcggaatgaagggcgagcgcagaagag
gcaagggacacgatggactgtatcagggcctgagcaccgccaccaaggat
acctatgatgccctgcacatgcaggccctgcctccaaga.

Modified T cells of the disclosure comprising/expressing a CSR of the disclosure improve the expansion of T cells when compared to those cells that do not comprise/express a CSR of the disclosure.

### Immune and Immune Precursor Cells

In certain embodiments, immune cells of the disclosure comprise lymphoid progenitor cells, natural killer (NK) cells, T lymphocytes (T-cell), stem memory T cells (T_{SCM} cells), central memory T cells (T_{CM}), stem cell-like T cells, B lymphocytes (B-cells), myeloid progenitor cells, neutrophils, basophils, eosinophils, monocytes, macrophages, platelets, erythrocytes, red blood cells (RBCs), megakaryocytes or osteoclasts.

In certain embodiments, immune precursor cells comprise any cells which can differentiate into one or more types of immune cells. In certain embodiments, immune precursor cells comprise multipotent stem cells that can self renew and develop into immune cells. In certain embodiments, immune precursor cells comprise hematopoietic stem cells (HSCs) or descendants thereof. In certain embodiments, immune precursor cells comprise precursor cells that can develop into immune cells. In certain embodiments, the immune precursor cells comprise hematopoietic progenitor cells (HPCs).

### Hematopoietic Stem Cells (HSCs)

Hematopoietic stem cells (HSCs) are multipotent, self-renewing cells. All differentiated blood cells from the lymphoid and myeloid lineages arise from HSCs. HSCs can be found in adult bone marrow, peripheral blood, mobilized peripheral blood, peritoneal dialysis effluent and umbilical cord blood.

HSCs of the disclosure may be isolated or derived from a primary or cultured stem cell. HSCs of the disclosure may be isolated or derived from an embryonic stem cell, a multipotent stem cell, a pluripotent stem cell, an adult stem cell, or an induced pluripotent stem cell (iPSC).

Immune precursor cells of the disclosure may comprise an HSC or an HSC descendent cell. Exemplary HSC descendent cells of the disclosure include, but are not limited to, multipotent stem cells, lymphoid progenitor cells, natural killer (NK) cells, T lymphocyte cells (T-cells), B lymphocyte cells (B-cells), myeloid progenitor cells, neutrophils, basophils, eosinophils, monocytes, and macrophages.

HSCs produced by the methods of the disclosure may retain features of “primitive” stem cells that, while isolated or derived from an adult stem cell and while committed to a single lineage, share characteristics of embryonic stem cells. For example, the “primitive” HSCs produced by the methods of the disclosure retain their “stemness” following division and do not differentiate. Consequently, as an adoptive cell therapy, the “primitive” HSCs produced by the methods of the disclosure not only replenish their numbers, but expand in vivo. “Primitive” HSCs produced by the methods of the disclosure may be therapeutically-effective when administered as a single dose. In some embodiments, primitive HSCs of the disclosure are CD34+. In some embodiments, primitive HSCs of the disclosure are CD34+ and CD38−. In some embodiments, primitive HSCs of the disclosure are CD34+, CD38− and CD90+. In some embodiments, primitive HSCs of the disclosure are CD34+, CD38−, CD90+ and CD45RA−. In some embodiments, primitive HSCs of the disclosure are CD34+, CD38−, CD90+, CD45RA−, and CD49f+. In some embodiments, the most primitive HSCs of the disclosure are CD34+, CD38−, CD90+, CD45RA−, and CD49f+.

In some embodiments of the disclosure, primitive HSCs, HSCs, and/or HSC descendent cells may be modified according to the methods of the disclosure to express an exogenous sequence (e.g. a chimeric antigen receptor or therapeutic protein). In some embodiments of the disclosure, modified primitive HSCs, modified HSCs, and/or modified HSC descendent cells may be forward differentiated to produce a modified immune cell including, but not limited to, a modified T cell, a modified natural killer cell and/or a modified B-cell of the disclosure.

### T Cells

Modified T cells of the disclosure may be derived from modified hematopoietic stem and progenitor cells (HSPCs) or modified HSCs.

Unlike traditional biologics and chemotherapeutics, modified-T cells of the disclosure possess the capacity to rapidly reproduce upon antigen recognition, thereby potentially obviating the need for repeat treatments. To achieve this, in some embodiments, modified-T cells of the disclosure not only drive an initial response, but also persist in the patient as a stable population of viable memory T cells to prevent potential relapses. Alternatively, in some embodiments, when it is not desired, modified-T cells of the disclosure do not persist in the patient.

Intensive efforts have been focused on the development of antigen receptor molecules that do not cause T cell exhaustion through antigen-independent (tonic) signaling, as well as of a modified-T cell product containing early memory T cells, especially stem cell memory (T_{SCM}) or stem cell-like T cells. Stem cell-like modified-T cells of the disclosure exhibit the greatest capacity for self-renewal and multipotent capacity to derive central memory (T_{CM}) T cells or T_{CM} like cells, effector memory (T_{EM}) and effector T cells (T_{E}), thereby producing better tumor eradication and long-term modified-T cell engraftment. A linear pathway of differentiation may be responsible for generating these cells: Naïve T cells (T_{N})>T_{SCM}>T_{CM}>T_{EM}>T_{E}>T_{TE}, whereby T_{N} is the parent precursor cell that directly gives rise to T_{SCM}, which then, in turn, directly gives rise to T_{CM}, etc. Compositions of T cells of the disclosure may comprise one or more of each parental T cell subset with T_{SCM} cells being the most abundant (e.g. T_{SCM}>T_{CM}>T_{EM}>T_{E}>T_{TE}).

In some embodiments of the methods of the disclosure, the immune cell precursor is differentiated into or is capable of differentiating into an early memory T cell, a stem cell like T-cell, a Naïve T cells (T_{N}), a T_{SCM}, a T_{CM}, a T_{EM}, a T_{E}, or a T_{TE}. In some embodiments, the immune cell precursor is a primitive HSC, an HSC, or a HSC descendent cell of the disclosure.

In some embodiments of the methods of the disclosure, the immune cell is an early memory T cell, a stem cell like T-cell, a Naïve T cells (T_{N}), a T_{SCM}, a T_{CM}, a T_{EM}, a T_{E}, or a T_{TE}.

In some embodiments of the methods of the disclosure, the immune cell is an early memory T cell.

In some embodiments of the methods of the disclosure, the immune cell is a stem cell like T-cell.

In some embodiments of the methods of the disclosure, the immune cell is a T_{SCM}.

In some embodiments of the methods of the disclosure, the immune cell is a T_{CM}.

In some embodiments of the methods of the disclosure, the methods modify and/or the methods produce a plurality of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells expresses one or more cell-surface marker(s) of an early memory T cell. In certain embodiments, the plurality of modified early memory T cells comprises at least one modified stem cell-like T cell. In certain embodiments, the plurality of modified early memory T cells comprises at least one modified T_{SCM}. In certain embodiments, the plurality of modified early memory T cells comprises at least one modified T_{CM}.

In some embodiments of the methods of the disclosure, the methods modify and/or the methods produce a plurality of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells expresses one or more cell-surface marker(s) of a stem cell-like T cell. In certain embodiments, the plurality of modified stem cell-like T cells comprises at least one modified T_{SCM}. In certain embodiments, the plurality of modified stem cell-like T cells comprises at least one modified T_{CM}.

In some embodiments of the methods of the disclosure, the methods modify and/or the methods produce a plurality of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells expresses one or more cell-surface marker(s) of a stem memory T cell (T_{SCM}). In certain embodiments, the cell-surface markers comprise CD62L and CD45RA. In certain embodiments, the cell-surface markers comprise one or more of CD62L, CD45RA, CD28, CCR7, CD127, CD45RO, CD95, CD95 and IL-2Rβ. In certain embodiments, the cell-surface markers comprise one or more of CD45RA, CD95, CCR7, and CD62L.

In some embodiments of the methods of the disclosure, the methods modify and/or the methods produce a plurality of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells expresses one or more cell-surface marker(s) of a central memory T cell (T_{CM}). In certain embodiments, the cell-surface markers comprise one or more of CD45RO, CD95, IL-2Rβ, CCR7, and CD62L.

In some embodiments of the methods of the disclosure, the methods modify and/or the methods produce a plurality of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells expresses one or more cell-surface marker(s) of a naïve T cell (T_{N}). In certain embodiments, the cell-surface markers comprise one or more of CD45RA, CCR7 and CD62L.

In some embodiments of the methods of the disclosure, the methods modify and/or the methods produce a plurality of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells expresses one or more cell-surface marker(s) of an effector T-cell (modified TEFF). In certain embodiments, the cell-surface markers comprise one or more of CD45RA, CD95, and IL-2Rβ.

In some embodiments of the methods of the disclosure, the methods modify and/or the methods produce a plurality of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells expresses one or more cell-surface marker(s) of a stem cell-like T cell, a stem memory T cell (T_{SCM}) or a central memory T cell (T_{CM}).

In some embodiments of the methods of the disclosure, a buffer comprises the immune cell or precursor thereof. The buffer maintains or enhances a level of cell viability and/or a stem-like phenotype of the immune cell or precursor thereof, including T-cells. In certain embodiments, the buffer maintains or enhances a level of cell viability and/or a stem-like phenotype of the primary human T cells prior to the nucleofection. In certain embodiments, the buffer maintains or enhances a level of cell viability and/or a stem-like phenotype of the primary human T cells during the nucleofection. In certain embodiments, the buffer maintains or enhances a level of cell viability and/or a stem-like phenotype of the primary human T cells following the nucleofection. In certain embodiments, the buffer comprises one or more of KCl, MgCl₂, ClNa, Glucose and Ca(NO₃)₂ in any absolute or relative abundance or concentration, and, optionally, the buffer further comprises a supplement selected from the group consisting of HEPES, Tris/HCl, and a phosphate buffer. In certain embodiments, the buffer comprises 5 mM KCl, 15 mM MgCl₂, 90 mM ClNa, 10 mM Glucose and 0.4 mM Ca(NO₃)₂. In certain embodiments, the buffer comprises 5 mM KCl, 15 mM MgCl₂, 90 mM ClNa, 10 mM Glucose and 0.4 mM Ca(NO₃)₂ and a supplement comprising 20 mM HEPES and 75 mM Tris/HCl. In certain embodiments, the buffer comprises 5 mM KCl, 15 mM MgCl₂, 90 mM ClNa, 10 mM Glucose and 0.4 mM Ca(NO₃)₂ and a supplement comprising 40 mM Na₂HPO₄/NaH₂PO₄ at pH 7.2. In certain embodiments, the composition comprising primary human T cells comprises 100 μl of the buffer and between 5×10⁶ and 25×10⁶ cells. In certain embodiments, the composition comprises a scalable ratio of 250×10⁶ primary human T cells per milliliter of buffer or other media during the introduction step.

In some embodiments of the methods of the disclosure, the methods comprise contacting an immune cell of the disclosure, including a T cell of the disclosure, and a T-cell expansion composition. In some embodiments of the methods of the disclosure, the step of introducing a transposon and/or transposase of the disclosure into an immune cell of the disclosure may further comprise contacting the immune cell and a T-cell expansion composition. In some embodiments, including those in which the introducing step of the methods comprises an electroporation or a nucleofection step, the electroporation or a nucleofection step may be performed with the immune cell contacting T-cell expansion composition of the disclosure.

In some embodiments of the methods of the disclosure, the T-cell expansion composition comprises, consists essentially of or consists of phosphorus; one or more of an octanoic acid, a palmitic acid, a linoleic acid, and an oleic acid; a sterol; and an alkane.

In certain embodiments of the methods of producing a modified T cell of the disclosure, the expansion supplement comprises one or more cytokine(s). The one or more cytokine(s) may comprise any cytokine, including but not limited to, lymphokines. Exemplary lympokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-15 (IL-15), interleukin-21 (IL-21), granulocyte-macrophage colony-stimulating factor (GM-CSF) and interferon-gamma (INFγ). The one or more cytokine(s) may comprise IL-2.

In some embodiments of the methods of the disclosure, the T-cell expansion composition comprises human serum albumin, recombinant human insulin, human transferrin, 2-Mercaptoethanol, and an expansion supplement. In certain embodiments of this method, the T-cell expansion composition further comprises one or more of octanoic acid, nicotinamide, 2,4,7,9-tetramethyl-5-decyn-4,7-diol (TMDD), diisopropyl adipate (DIPA), n-butyl-benzenesulfonamide, 1,2-benzenedicarboxylic acid, bis(2-methylpropyl) ester, palmitic acid, linoleic acid, oleic acid, stearic acid hydrazide, oleamide, a sterol and an alkane. In certain embodiments of this method, the T-cell expansion composition further comprises one or more of octanoic acid, palmitic acid, linoleic acid, oleic acid and a sterol. In certain embodiments of this method, the T-cell expansion composition further comprises one or more of octanoic acid at a concentration of between 0.9 mg/kg to 90 mg/kg, inclusive of the endpoints; palmitic acid at a concentration of between 0.2 mg/kg to 20 mg/kg, inclusive of the endpoints; linoleic acid at a concentration of between 0.2 mg/kg to 20 mg/kg, inclusive of the endpoints; oleic acid at a concentration of 0.2 mg/kg to 20 mg/kg, inclusive of the endpoints; and a sterol at a concentration of about 0.1 mg/kg to 10 mg/kg, inclusive of the endpoints. In certain embodiments of this method, the T-cell expansion composition further comprises one or more of octanoic acid at a concentration of about 9 mg/kg, palmitic acid at a concentration of about 2 mg/kg, linoleic acid at a concentration of about 2 mg/kg, oleic acid at a concentration of about 2 mg/kg and a sterol at a concentration of about 1 mg/kg. In certain embodiments of this method, the T-cell expansion composition further comprises one or more of octanoic acid at a concentration of between 6.4 μmol/kg and 640 μmol/kg, inclusive of the endpoints; palmitic acid at a concentration of between 0.7 μmol/kg and 70 μmol/kg, inclusive of the endpoints; linoleic acid at a concentration of between 0.75 μmol/kg and 75 μmol/kg, inclusive of the endpoints; oleic acid at a concentration of between 0.75 μmol/kg and 75 μmol/kg, inclusive of the endpoints; and a sterol at a concentration of between 0.25 μmol/kg and 25 μmol/kg, inclusive of the endpoints. In certain embodiments of this method, the T-cell expansion composition further comprises one or more of octanoic acid at a concentration of about 64 μmol/kg, palmitic acid at a concentration of about 7 μmol/kg, linoleic acid at a concentration of about 7.5 μmol/kg, oleic acid at a concentration of about 7.5 μmol/kg and a sterol at a concentration of about 2.5 μmol/kg.

In certain embodiments, the T-cell expansion composition comprises one or more of human serum albumin, recombinant human insulin, human transferrin, 2-Mercaptoethanol, and an expansion supplement to produce a plurality of expanded modified T-cells, wherein at least 2% of the plurality of modified T-cells expresses one or more cell-surface marker(s) of an early memory T cell, a stem cell-like T cell, a stem memory T cell (T_{SCM}) and/or a central memory T cell (T_{CM}). In certain embodiments, the T-cell expansion composition comprises or further comprises one or more of octanoic acid, nicotinamide, 2,4,7,9-tetramethyl-5-decyn-4,7-diol (TMDD), diisopropyl adipate (DIPA), n-butyl-benzenesulfonamide, 1,2-benzenedicarboxylic acid, bis(2-methylpropyl) ester, palmitic acid, linoleic acid, oleic acid, stearic acid hydrazide, oleamide, a sterol and an alkane. In certain embodiments, the T-cell expansion composition comprises one or more of octanoic acid, palmitic acid, linoleic acid, oleic acid and a sterol (e.g. cholesterol). In certain embodiments, the T-cell expansion composition comprises one or more of octanoic acid at a concentration of between 0.9 mg/kg to 90 mg/kg, inclusive of the endpoints; palmitic acid at a concentration of between 0.2 mg/kg to 20 mg/kg, inclusive of the endpoints; linoleic acid at a concentration of between 0.2 mg/kg to 20 mg/kg, inclusive of the endpoints; oleic acid at a concentration of 0.2 mg/kg to 20 mg/kg, inclusive of the endpoints; and a sterol at a concentration of about 0.1 mg/kg to 10 mg/kg, inclusive of the endpoints (wherein mg/kg=parts per million). In certain embodiments, the T-cell expansion composition comprises one or more of octanoic acid at a concentration of about 9 mg/kg, palmitic acid at a concentration of about 2 mg/kg, linoleic acid at a concentration of about 2 mg/kg, oleic acid at a concentration of about 2 mg/kg, and a sterol at a concentration of about 1 mg/kg (wherein mg/kg=parts per million). In certain embodiments, the T-cell expansion composition comprises one or more of octanoic acid at a concentration of 9.19 mg/kg, palmitic acid at a concentration of 1.86 mg/kg, linoleic acid at a concentration of about 2.12 mg/kg, oleic acid at a concentration of about 2.13 mg/kg, and a sterol at a concentration of about 1.01 mg/kg (wherein mg/kg=parts per million). In certain embodiments, the T-cell expansion composition comprises octanoic acid at a concentration of 9.19 mg/kg, palmitic acid at a concentration of 1.86 mg/kg, linoleic acid at a concentration of 2.12 mg/kg, oleic acid at a concentration of about 2.13 mg/kg, and a sterol at a concentration of 1.01 mg/kg (wherein mg/kg=parts per million). In certain embodiments, the T-cell expansion composition comprises one or more of octanoic acid at a concentration of between 6.4 μmol/kg and 640 μmol/kg, inclusive of the endpoints; palmitic acid at a concentration of between 0.7 μmol/kg and 70 μmol/kg, inclusive of the endpoints; linoleic acid at a concentration of between 0.75 μmol/kg and 75 μmol/kg, inclusive of the endpoints; oleic acid at a concentration of between 0.75 μmol/kg and 75 μmol/kg, inclusive of the endpoints; and a sterol at a concentration of between 0.25 μmol/kg and 25 μmol/kg, inclusive of the endpoints. In certain embodiments, the T-cell expansion composition comprises one or more of octanoic acid at a concentration of about 64 μmol/kg, palmitic acid at a concentration of about 7 μmol/kg, linoleic acid at a concentration of about 7.5 μmol/kg, oleic acid at a concentration of about 7.5 μmol/kg and a sterol at a concentration of about 2.5 μmol/kg. In certain embodiments, the T-cell expansion composition comprises one or more of octanoic acid at a concentration of about 63.75 μmol/kg, palmitic acid at a concentration of about 7.27 μmol/kg, linoleic acid at a concentration of about 7.57 μmol/kg, oleic acid at a concentration of about 7.56 μmol/kg and a sterol at a concentration of about 2.61 μmol/kg. In certain embodiments, the T-cell expansion composition comprises octanoic acid at a concentration of about 63.75 μmol/kg, palmitic acid at a concentration of about 7.27 μmol/kg, linoleic acid at a concentration of about 7.57 μmol/kg, oleic acid at a concentration of 7.56 μmol/kg and a sterol at a concentration of 2.61 μmol/kg.

As used herein, the terms “supplemented T-cell expansion composition” or “T-cell expansion composition” may be used interchangeably with a media comprising one or more of human serum albumin, recombinant human insulin, human transferrin, 2-Mercaptoethanol, and an expansion supplement at 37° C. Alternatively, or in addition, the terms “supplemented T-cell expansion composition” or “T-cell expansion composition” may be used interchangeably with a media comprising one or more of phosphorus, an octanoic fatty acid, a palmitic fatty acid, a linoleic fatty acid and an oleic acid. In certain embodiments, the media comprises an amount of phosphorus that is 10-fold higher than may be found in, for example, Iscove's Modified Dulbecco's Medium ((IMDM); available at ThermoFisher Scientific as Catalog number 12440053).

As used herein, the terms “supplemented T-cell expansion composition” or “T-cell expansion composition” may be used interchangeably with a media comprising one or more of human serum albumin, recombinant human insulin, human transferrin, 2-Mercaptoethanol, Iscove's MDM, and an expansion supplement at 37° C. Alternatively, or in addition, the terms “supplemented T-cell expansion composition” or “T-cell expansion composition” may be used interchangeably with a media comprising one or more of the following elements: boron, sodium, magnesium, phosphorus, potassium, and calcium. In certain embodiments, the terms “supplemented T-cell expansion composition” or “T-cell expansion composition” may be used interchangeably with a media comprising one or more of the following elements present in the corresponding average concentrations: boron at 3.7 mg/L, sodium at 3000 mg/L, magnesium at 18 mg/L, phosphorus at 29 mg/L, potassium at 15 mg/L and calcium at 4 mg/L.

As used herein, the terms “supplemented T-cell expansion composition” or “T-cell expansion composition” may be used interchangeably with a media comprising one or more of human serum albumin, recombinant human insulin, human transferrin, 2-Mercaptoethanol, and an expansion supplement at 37° C. Alternatively, or in addition, the terms “supplemented T-cell expansion composition” or “T-cell expansion composition” may be used interchangeably with a media comprising one or more of the following components: octanoic acid (CAS No. 124-07-2), nicotinamide (CAS No. 98-92-0), 2,4,7,9-tetramethyl-5-decyn-4,7-diol (TMDD) (CAS No. 126-86-3), diisopropyl adipate (DIPA) (CAS No. 6938-94-9), n-butyl-benzenesulfonamide (CAS No. 3622-84-2), 1,2-benzenedicarboxylic acid, bis(2-methylpropyl) ester (CAS No. 84-69-5), palmitic acid (CAS No. 57-10-3), linoleic acid (CAS No. 60-33-3), oleic acid (CAS No. 112-80-1), stearic acid hydrazide (CAS No. 4130-54-5), oleamide (CAS No. 3322-62-1), sterol (e.g., cholesterol) (CAS No. 57-88-5), and alkanes (e.g., nonadecane) (CAS No. 629-92-5). In certain embodiments, the terms “supplemented T-cell expansion composition” or “T-cell expansion composition” may be used interchangeably with a media comprising one or more of the following components: octanoic acid (CAS No. 124-07-2), nicotinamide (CAS No. 98-92-0), 2,4,7,9-tetramethyl-5-decyn-4,7-diol (TMDD) (CAS No. 126-86-3), diisopropyl adipate (DIPA) (CAS No. 6938-94-9), n-butyl-benzenesulfonamide (CAS No. 3622-84-2), 1,2-benzenedicarboxylic acid, bis(2-methylpropyl) ester (CAS No. 84-69-5), palmitic acid (CAS No. 57-10-3), linoleic acid (CAS No. 60-33-3), oleic acid (CAS No. 112-80-1), stearic acid hydrazide (CAS No. 4130-54-5), oleamide (CAS No. 3322-62-1), sterol (e.g., cholesterol) (CAS No. 57-88-5), alkanes (e.g., nonadecane) (CAS No. 629-92-5), and phenol red (CAS No. 143-74-8). In certain embodiments, the terms “supplemented T-cell expansion composition” or “T-cell expansion composition” may be used interchangeably with a media comprising one or more of the following components: octanoic acid (CAS No. 124-07-2), nicotinamide (CAS No. 98-92-0), 2,4,7,9-tetramethyl-5-decyn-4,7-diol (TMDD) (CAS No. 126-86-3), diisopropyl adipate (DIPA) (CAS No. 6938-94-9), n-butyl-benzenesulfonamide (CAS No. 3622-84-2), 1,2-benzenedicarboxylic acid, bis(2-methylpropyl) ester (CAS No. 84-69-5), palmitic acid (CAS No. 57-10-3), linoleic acid (CAS No. 60-33-3), oleic acid (CAS No. 112-80-1), stearic acid hydrazide (CAS No. 4130-54-5), oleamide (CAS No. 3322-62-1), phenol red (CAS No. 143-74-8) and lanolin alcohol.

In certain embodiments, the terms “supplemented T-cell expansion composition” or “T-cell expansion composition” may be used interchangeably with a media comprising one or more of human serum albumin, recombinant human insulin, human transferrin, 2-Mercaptoethanol, and an expansion supplement at 37° C. Alternatively, or in addition, the terms “supplemented T-cell expansion composition” or “T-cell expansion composition” may be used interchangeably with a media comprising one or more of the following ions: sodium, ammonium, potassium, magnesium, calcium, chloride, sulfate and phosphate.

As used herein, the terms “supplemented T-cell expansion composition” or “T-cell expansion composition” may be used interchangeably with a media comprising one or more of human serum albumin, recombinant human insulin, human transferrin, 2-Mercaptoethanol, and an expansion supplement at 37° C. Alternatively, or in addition, the terms “supplemented T-cell expansion composition” or “T-cell expansion composition” may be used interchangeably with a media comprising one or more of the following free amino acids: histidine, asparagine, serine, glutamate, arginine, glycine, aspartic acid, glutamic acid, threonine, alanine, proline, cysteine, lysine, tyrosine, methionine, valine, isoleucine, leucine, phenylalanine and tryptophan. In certain embodiments, the terms “supplemented T-cell expansion composition” or “T-cell expansion composition” may be used interchangeably with a media comprising one or more of the following free amino acids in the corresponding average mole percentages: histidine (about 1%), asparagine (about 0.5%), serine (about 1.5%), glutamine (about 67%), arginine (about 1.5%), glycine (about 1.5%), aspartic acid (about 1%), glutamic acid (about 2%), threonine (about 2%), alanine (about 1%), proline (about 1.5%), cysteine (about 1.5%), lysine (about 3%), tyrosine (about 1.5%), methionine (about 1%), valine (about 3.5%), isoleucine (about 3%), leucine (about 3.5%), phenylalanine (about 1.5%) and tryptophan (about 0.5%). In certain embodiments, the terms “supplemented T-cell expansion composition” or “T-cell expansion composition” may be used interchangeably with a media comprising one or more of the following free amino acids in the corresponding average mole percentages: histidine (about 0.78%), asparagine (about 0.4%), serine (about 1.6%), glutamine (about 67.01%), arginine (about 1.67%), glycine (about 1.72%), aspartic acid (about 1.00%), glutamic acid (about 1.93%), threonine (about 2.38%), alanine (about 1.11%), proline (about 1.49%), cysteine (about 1.65%), lysine (about 2.84%), tyrosine (about 1.62%), methionine (about 0.85%), valine (about 3.45%), isoleucine (about 3.14%), leucine (about 3.3%), phenylalanine (about 1.64%) and tryptophan (about 0.37%).

As used herein, the terms “supplemented T-cell expansion composition” or “T-cell expansion composition” may be used interchangeably with a media comprising one or more of human serum albumin, recombinant human insulin, human transferrin, 2-Mercaptoethanol, Iscove's MDM, and an expansion supplement at 37° C. Alternatively, or in addition, the terms “supplemented T-cell expansion composition” or “T-cell expansion composition” may be used interchangeably with a media comprising one or more of phosphorus, an octanoic fatty acid, a palmitic fatty acid, a linoleic fatty acid and an oleic acid. In certain embodiments, the media comprises an amount of phosphorus that is 10-fold higher than may be found in, for example, Iscove's Modified Dulbecco's Medium ((IMDM); available at ThermoFisher Scientific as Catalog number 12440053).

In certain embodiments, the terms “supplemented T-cell expansion composition” or “T-cell expansion composition” may be used interchangeably with a media comprising one or more of octanoic acid, palmitic acid, linoleic acid, oleic acid and a sterol (e.g. cholesterol). In certain embodiments, the terms “supplemented T-cell expansion composition” or “T-cell expansion composition” may be used interchangeably with a media comprising one or more of octanoic acid at a concentration of between 0.9 mg/kg to 90 mg/kg, inclusive of the endpoints; palmitic acid at a concentration of between 0.2 mg/kg to 20 mg/kg, inclusive of the endpoints; linoleic acid at a concentration of between 0.2 mg/kg to 20 mg/kg, inclusive of the endpoints; oleic acid at a concentration of 0.2 mg/kg to 20 mg/kg, inclusive of the endpoints; and a sterol at a concentration of about 0.1 mg/kg to 10 mg/kg, inclusive of the endpoints (wherein mg/kg=parts per million). In certain embodiments, the terms “supplemented T-cell expansion composition” or “T-cell expansion composition” may be used interchangeably with a media comprising one or more of octanoic acid at a concentration of about 9 mg/kg, palmitic acid at a concentration of about 2 mg/kg, linoleic acid at a concentration of about 2 mg/kg, oleic acid at a concentration of about 2 mg/kg, and a sterol at a concentration of about 1 mg/kg (wherein mg/kg=parts per million). In certain embodiments, the terms “supplemented T-cell expansion composition” or “T-cell expansion composition” may be used interchangeably with a media comprising one or more of octanoic acid at a concentration of 9.19 mg/kg, palmitic acid at a concentration of 1.86 mg/kg, linoleic acid at a concentration of about 2.12 mg/kg, oleic acid at a concentration of about 2.13 mg/kg, and a sterol at a concentration of about 1.01 mg/kg (wherein mg/kg=parts per million). In certain embodiments, the terms “supplemented T-cell expansion composition” or “T-cell expansion composition” may be used interchangeably with a media comprising one or more of octanoic acid at a concentration of 9.19 mg/kg, palmitic acid at a concentration of 1.86 mg/kg, linoleic acid at a concentration of 2.12 mg/kg, oleic acid at a concentration of about 2.13 mg/kg, and a sterol at a concentration of 1.01 mg/kg (wherein mg/kg=parts per million). In certain embodiments, the terms “supplemented T-cell expansion composition” or “T-cell expansion composition” may be used interchangeably with a media comprising one or more of octanoic acid at a concentration of between 6.4 μmol/kg and 640 μmol/kg, inclusive of the endpoints; palmitic acid at a concentration of between 0.7 μmol/kg and 70 μmol/kg, inclusive of the endpoints; linoleic acid at a concentration of between 0.75 μmol/kg and 75 μmol/kg, inclusive of the endpoints; oleic acid at a concentration of between 0.75 μmol/kg and 75 μmol/kg, inclusive of the endpoints; and a sterol at a concentration of between 0.25 μmol/kg and 25 μmol/kg, inclusive of the endpoints. In certain embodiments, the terms “supplemented T-cell expansion composition” or “T-cell expansion composition” may be used interchangeably with a media comprising one or more of octanoic acid at a concentration of about 64 μmol/kg, palmitic acid at a concentration of about 7 μmol/kg, linoleic acid at a concentration of about 7.5 μmol/kg, oleic acid at a concentration of about 7.5 μmol/kg and a sterol at a concentration of about 2.5 μmol/kg.

In certain embodiments, the terms “supplemented T-cell expansion composition” or “T-cell expansion composition” may be used interchangeably with a media comprising one or more of octanoic acid at a concentration of about 63.75 μmol/kg, palmitic acid at a concentration of about 7.27 μmol/kg, linoleic acid at a concentration of about 7.57 μmol/kg, oleic acid at a concentration of about 7.56 μmol/kg and a sterol at a concentration of about 2.61 μmol/kg. In certain embodiments, the terms “supplemented T-cell expansion composition” or “T-cell expansion composition” may be used interchangeably with a media comprising one or more of octanoic acid at a concentration of about 63.75 μmol/kg, palmitic acid at a concentration of about 7.27 μmol/kg, linoleic acid at a concentration of about 7.57 μmol/kg, oleic acid at a concentration of 7.56 μmol/kg and a sterol at a concentration of 2.61 μmol/kg.

In certain embodiments of the methods of producing a modified T cell (e.g. a stem cell-like T cell, a T_{SCM} and/or a T_{CM}) of the disclosure, the method comprises contacting a modified T cell and an inhibitor of the P13K-Akt-mTOR pathway. Modified T-cells of the disclosure, including modified stem cell-like T cells, T_{SCM} and/or T_{CM} of the disclosure, may be incubated, cultured, grown, stored, or otherwise, combined at any step in the methods of the procedure with a growth medium comprising one or more inhibitors a component of a PI3K pathway. Exemplary inhibitors a component of a PI3K pathway include, but are not limited to, an inhibitor of GSK3β such as TWS119 (also known as GSK 3B inhibitor XII; CAS Number 601514-19-6 having a chemical formula C₁₈H₁₄N₄O₂). Exemplary inhibitors of a component of a PI3K pathway include, but are not limited to, bb007 (BLUEBIRDBIO™). Additional Exemplary inhibitors of a component of a PI3K pathway include, but are not limited to, an allosteric Akt inhibitor VIII (also referred to as Akti-1/2 having Compound number 10196499), ATP competitive inhibitors (Orthosteric inhibitors targeting the ATP-binding pocket of the protein kinase B (Akt)), Isoquinoline-5-sulfonamides (H-8, H-89, and NL-71-101), Azepane derivatives (A series of structures derived from (−)-balanol), Aminofurazans (GSK690693), Heterocyclic rings (7-azaindole, 6-phenylpurine derivatives, pyrrolo[2,3-d]pyrimidine derivatives, CCT128930, 3-aminopyrrolidine, anilinotriazole derivatives, spiroindoline derivatives, AZD5363, ipatasertib (GDC-0068, RG7440), A-674563, and A-443654), Phenylpyrazole derivatives (AT7867 and AT13148), Thiophenecarboxamide derivatives (Afuresertib (GSK2110183), 2-pyrimidyl-5-amidothiophene derivative (DC120), uprosertib (GSK2141795)), Allosteric inhibitors (Superior to orthosteric inhibitors providing greater specificity, reduced side-effects and less toxicity), 2,3-diphenylquinoxaline analogues (2,3-diphenylquinoxaline derivatives, triazolo[3,4-f][1,6]naphthyridin-3(2H)-one derivative (MK-2206)), Alkylphospholipids (Edelfosine (1-O-octadecyl-2-O-methyl-rac-glycero-3-phosphocholine, ET-18-OCH₃) ilmofosine (BM 41.440), miltefosine (hexadecylphosphocholine, HePC), perifosine (D-21266), erucylphosphocholine (ErPC), erufosine (ErPC3, erucylphosphohomocholine), Indole-3-carbinol analogues (Indole-3-carbinol, 3-chloroacetylindole, diindolylmethane, diethyl 6-methoxy-5,7-dihydroindolo [2,3-b]carbazole-2,10-dicarboxylate (SR13668), OSU-A9), Sulfonamide derivatives (PH-316 and PHT-427), Thiourea derivatives (PIT-1, PIT-2, DM-PIT-1, N-[(1-methyl-1H-pyrazol-4-yl)carbonyl]-N-(3-bromophenyl)-thiourea), Purine derivatives (Triciribine (TCN, NSC 154020), triciribine mono-phosphate active analogue (TCN-P), 4-amino-pyrido[2,3-d]pyrimidine derivative API-1, 3-phenyl-3H-imidazo[4,5-b]pyridine derivatives, ARQ 092), BAY 1125976, 3-methyl-xanthine, quinoline-4-carboxamide and 2-[4-(cyclohexa-1,3-dien-1-yl)-1H-pyrazol-3-yl]phenol, 3-oxo-tirucallic acid, 3σ- and 3β-acetoxy-tirucallic acids, acetoxy-tirucallic acid, and irreversible inhibitors (antibiotics, Lactoquinomycin, Frenolicin B, kalafungin, medermycin, Boc-Phe-vinyl ketone, 4-hydroxynonenal (4-HNE), 1,6-naphthyridinone derivatives, and imidazo-1,2-pyridine derivatives).

In certain embodiments of the methods of producing a modified T cell (e.g. a stem cell-like T cell, a T_{SCM} and/or a T_{CM}) of the disclosure, the method comprises contacting a modified T cell and an inhibitor of T cell effector differentiation. Exemplary inhibitors of T cell effector differentiation include, but are not limited to, a BET inhibitor (e.g. JQ1, a hienotriazolodiazepine) and/or an inhibitor of the BET family of proteins (e.g. BRD2, BRD3, BRD4, and BRDT).

In certain embodiments of the methods of producing a modified T cell (e.g. a stem cell-like T cell, a T_{SCM} and/or a T_{CM}) of the disclosure, the method comprises contacting a modified T cell and an agent that reduces nucleo-cytoplasmic Acetyl-CoA. Exemplary agents that reduce nucleo-cytoplasmic Acetyl-CoA include, but are not limited to, 2-hydroxy-citrate (2-HC) as well as agents that increase expression of Acss1.

In certain embodiments of the methods of producing a modified T cell (e.g. a stem cell-like T cell, a TSCM and/or a TCM) of the disclosure, the method comprises contacting a modified T cell and a composition comprising a histone deacetylase (HDAC) inhibitor. In some embodiments, the composition comprising an HDAC inhibitor comprises or consists of valproic acid, Sodium Phenylbutyrate (NaPB) or a combination thereof. In some embodiments, the composition comprising an HDAC inhibitor comprises or consists of valproic acid. In some embodiments, the composition comprising an HDAC inhibitor comprises or consists of Sodium Phenylbutyrate (NaPB).

In certain embodiments of the methods of producing a modified T cell (e.g. a stem cell-like T cell, a T_{SCM} and/or a T_{CM}) of the disclosure, the activation supplement may comprise one or more cytokine(s). The one or more cytokine(s) may comprise any cytokine, including but not limited to, lymphokines. Exemplary lympokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-15 (IL-15), interleukin-21 (IL-21), granulocyte-macrophage colony-stimulating factor (GM-CSF) and interferon-gamma (INFγ). The one or more cytokine(s) may comprise IL-2.

In certain embodiments of the methods of producing a modified T cell (e.g. a stem cell-like T cell, a T_{SCM} and/or a T_{CM}) of the disclosure, the activation supplement may comprise one or more activator complexes. Exemplary and nonlimiting activator complexes may comprise a monomeric, dimeric, trimeric or tetrameric antibody complex that binds one or more of CD3, CD28, and CD2. In some embodiments, the activation supplement comprises or consists of an activator complex that comprises a human, a humanized or a recombinant or a chimeric antibody. In some embodiments, the activation supplement comprises or consists of an activator complex that binds CD3 and CD28. In some embodiments, the activation supplement comprises or consists of an activator complex that binds CD3, CD28 and CD2.

### Natural Killer (NK) Cells

In certain embodiments, the modified immune or immune precursor cells of the disclosure are natural killer (NK) cells. In certain embodiments, NK cells are cytotoxic lymphocytes that differentiate from lymphoid progenitor cells.

Modified NK cells of the disclosure may be derived from modified hematopoietic stem and progenitor cells (HSPCs) or modified HSCs.

In certain embodiments, non-activated NK cells are derived from CD3-depleted leukopheresis (containing CD14/CD19/CD56+ cells).

In certain embodiments, NK cells are electroporated using a Lonza 4D nucleofector or BTX ECM 830 (500V, 700 usec pulse length, 0.2 mm electrode gap, one pulse). All Lonza 4D nucleofector programs are contemplated as within the scope of the methods of the disclosure.

In certain embodiments, 5×10E6 cells were electroporated per electroporation in 100 μL P3 buffer in cuvettes. However, this ratio of cells per volume is scalable for commercial manufacturing methods.

In certain embodiments, NK cells were stimulated by co-culture with an additional cell line. In certain embodiments, the additional cell line comprises artificial antigen presenting cells (aAPCs). In certain embodiments, stimulation occurs at day 1, 2, 3, 4, 5, 6, or 7 following electroporation. In certain embodiments, stimulation occurs at day 2 following electroporation.

In certain embodiments, NK cells express CD56.

### B Cells

In certain embodiments, the modified immune or immune precursor cells of the disclosure are B cells. B cells are a type of lymphocyte that express B cell receptors on the cell surface. B cell receptors bind to specific antigens.

Modified B cells of the disclosure may be derived from modified hematopoietic stem and progenitor cells (HSPCs) or modified HSCs.

In certain embodiments, HSPCs are modified using the methods of the disclosure, and then primed for B cell differentiation in presence of human IL-3, Flt3L, TPO, SCF, and G-CSF for at least 3 days, at least 4 days, at least 5 days, at least 6 days or at least 7 days. In certain embodiments, HSPCs are modified using the methods of the disclosure, and then primed for B cell differentiation in presence of human IL-3, Flt3L, TPO, SCF, and G-CSF for 5 days.

In certain embodiments, following priming, modified HSPC cells are transferred to a layer of feeder cells and fed bi-weekly, along with transfer to a fresh layer of feeders once per week. In certain embodiments, the feeder cells are MS-5 feeder cells.

In certain embodiments, modified HSPC cells are cultured with MS-5 feeder cells for at least 7, 14, 21, 28, 30, 33, 35, 42 or 48 days. In certain embodiments, modified HSPC cells were cultured with MS-5 feeder cells for 33 days.

### Transposition Systems

Exemplary transposon/transposase systems of the disclosure include, but are not limited to, piggyBac transposons and transposases, Sleeping Beauty transposons and transposases, Helraiser transposons and transposases, Tol2 transposons and transposases and TcBuster transposons and transposases.

The piggyBac transposase recognizes transposon-specific inverted terminal repeat sequences (ITRs) on the ends of the transposon, and moves the contents between the ITRs into TTAA chromosomal sites. The piggyBac transposon system has no payload limit for the genes of interest that can be included between the ITRs. In certain embodiments, and, in particular, those embodiments wherein the transposon is a piggyBac transposon, the transposase is a piggyBac™ or a Super piggyBac™ (SPB) transposase. In certain embodiments, and, in particular, those embodiments wherein the transposase is a Super piggyBac™ (SPB) transposase, the sequence encoding the transposase is an mRNA sequence.

In certain embodiments of the methods of the disclosure, the transposase enzyme is a piggyBac™ (PB) transposase enzyme. The piggyBac (PB) transposase enzyme may comprise or consist of an amino acid sequence at least 75%, 80%, 85%, 90%, 95%, 99% or any percentage in between identical to:

```
(SEQ ID NO: 14487)
    1 MGSSLDDEHI LSALLQSDDE LVGEDSDSEI SDHVSEDDVQ SDTEEAFIDE VHEVQPTSSG
  61 SEILDEQNVI EQPGSSLASN RILTLPQRTI RGKNKHCWST SKSTRRSRVS ALNIVRSQRG
121 PTRMCRNIYD PLLCFKLFFT DEIISEIVKW TNAEISLKRR ESMTGATFRD TNEDEIYAFF
181 GILVMTAVRK DNHMSTDDLF DRSLSMVYVS VMSRDRFDFL IRCLRMDDKS IRPTLRENDV
241 FTPVRKIWDL FIHQCIQNYT PGAHLTIDEQ LLGFRGRCPF RMYIPNKPSK YGIKILMMCD
301 SGTKYMINGM PYLGRGTQTN GVPLGEYYVK ELSKPVHGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDTLD QMCSVMTCSR KTNRWPMALL YGMINIACIN
481 SFIIYSHNVS SKGEKVQSRK KFMRNLYMSL TSSFMRKRLE APTLKRYLRD NISNILPNEV
541 PGTSDDSTEE PVMKKRTYCT YCPSKIRRKA NASCKKCKKV ICREHNIDMC QSCF.
```

In certain embodiments of the methods of the disclosure, the transposase enzyme is a piggyBac™ (PB) transposase enzyme that comprises or consists of an amino acid sequence having an amino acid substitution at one or more of positions 30, 165, 282, or 538 of the sequence:

```
(SEQ ID NO: 14487)
    1 MGSSLDDEHI LSALLQSDDE LVGEDSDSEI SDHVSEDDVQ SDTEEAFIDE VHEVQPTSSG
  61 SEILDEQNVI EQPGSSLASN RILTLPQRTI RGKNKHCWST SKSTRRSRVS ALNIVRSQRG
121 PTRMCRNIYD PLLCFKLFFT DEIISEIVKW TNAEISLKRR ESMTGATFRD TNEDEIYAFF
181 GILVMTAVRK DNHMSTDDLF DRSLSMVYVS VMSRDRFDFL IRCLRMDDKS IRPTLRENDV
241 FTPVRKIWDL FIHQCIQNYT PGAHLTIDEQ LLGFRGRCPF RMYIPNKPSK YGIKILMMCD
301 SGTKYMINGM PYLGRGTQTN GVPLGEYYVK ELSKPVHGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDTLD QMCSVMTCSR KTNRWPMALL YGMINIACIN
481 SFIIYSHNVS SKGEKVQSRK KFMRNLYMSL TSSFMRKRLE APTLKRYLRD NISNILPNEV
541 PGTSDDSTEE PVMKKRTYCT YCPSKIRRKA NASCKKCKKV ICREHNIDMC QSCF.
```

In certain embodiments, the transposase enzyme is a piggyBac™ (PB) transposase enzyme that comprises or consists of an amino acid sequence having an amino acid substitution at two or more of positions 30, 165, 282, or 538 of the sequence of SEQ ID NO: 14487. In certain embodiments, the transposase enzyme is a piggyBac™ (PB) transposase enzyme that comprises or consists of an amino acid sequence having an amino acid substitution at three or more of positions 30, 165, 282, or 538 of the sequence of SEQ ID NO: 14487. In certain embodiments, the transposase enzyme is a piggyBac™ (PB) transposase enzyme that comprises or consists of an amino acid sequence having an amino acid substitution at each of the following positions 30, 165, 282, and 538 of the sequence of SEQ ID NO: 14487. In certain embodiments, the amino acid substitution at position 30 of the sequence of SEQ ID NO: 14487 is a substitution of a valine (V) for an isoleucine (I). In certain embodiments, the amino acid substitution at position 165 of the sequence of SEQ ID NO: 14487 is a substitution of a serine (S) for a glycine (G). In certain embodiments, the amino acid substitution at position 282 of the sequence of SEQ ID NO: 14487 is a substitution of a valine (V) for a methionine (M). In certain embodiments, the amino acid substitution at position 538 of the sequence of SEQ ID NO: 14487 is a substitution of a lysine (K) for an asparagine (N).

In certain embodiments of the methods of the disclosure, the transposase enzyme is a Super piggyBac™ (SPB) transposase enzyme. In certain embodiments, the Super piggyBac™ (SPB) transposase enzymes of the disclosure may comprise or consist of the amino acid sequence of the sequence of SEQ ID NO: 14487 wherein the amino acid substitution at position 30 is a substitution of a valine (V) for an isoleucine (I), the amino acid substitution at position 165 is a substitution of a serine (S) for a glycine (G), the amino acid substitution at position 282 is a substitution of a valine (V) for a methionine (M), and the amino acid substitution at position 538 is a substitution of a lysine (K) for an asparagine (N). In certain embodiments, the Super piggyBac™ (SPB) transposase enzyme may comprise or consist of an amino acid sequence at least 75%, 80%, 85%, 90%, 95%, 99% or any percentage in between identical to:

```
(SEQ ID NO: 14484)
    1 MGSSLDDEHI LSALLQSDDE LVGEDSDSEV SDHVSEDDVQ SDTEEAFIDE VHEVQPTSSG
  61 SEILDEQNVI EQPGSSLASN RILTLPQRTI RGKNKHCWST SKSTRRSRVS ALNIVRSQRG
121 PTRMCRNIYD PLLCFKLFFT DEIISEIVKW TNAEISLKRR ESMTSATFRD TNEDEIYAFF
181 GILVMTAVRK DNHMSTDDLF DRSLSMVYVS VMSRDRFDFL IRCLRMDDKS IRPTLRENDV
241 FTPVRKIWDL FIHQCIQNYT PGAHLTIDEQ LLGFRGRCPF RVYIPNKPSK YGIKILMMCD
301 SGTKYMINGM PYLGRGTQTN GVPLGEYYVK ELSKPVHGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDTLD QMCSVMTCSR KTNRWPMALL YGMINIACIN
481 SFIIYSHNVS SKGEKVQSRK KFMRNLYMSL TSSFMRKRLE APTLKRYLRD NISNILPKEV
541 PGTSDDSTEE PVMKKRTYCT YCPSKIRRKA NASCKKCKKV ICREHNIDMC QSCF.
```

In certain embodiments of the methods of the disclosure, including those embodiments wherein the transposase comprises the above-described mutations at positions 30, 165, 282 and/or 538, the piggyBac™ or Super piggyBac™ transposase enzyme may further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 14487 or SEQ ID NO: 14484. In certain embodiments, including those embodiments wherein the transposase comprises the above-described mutations at positions 30, 165, 282 and/or 538, the piggyBac™ or Super piggyBac™ transposase enzyme may further comprise an amino acid substitution at one or more of positions 46, 119, 125, 177, 180, 185, 187, 200, 207, 209, 226, 235, 240, 241, 243, 296, 298, 311, 315, 319, 327, 328, 340, 421, 436, 456, 470, 485, 503, 552 and 570. In certain embodiments, the amino acid substitution at position 3 of SEQ ID NO: 14487 or SEQ ID NO: 14484 is a substitution of an asparagine (N) for a serine (S). In certain embodiments, the amino acid substitution at position 46 of SEQ ID NO: 14487 or SEQ ID NO: 14484 is a substitution of a serine (S) for an alanine (A). In certain embodiments, the amino acid substitution at position 46 of SEQ ID NO: 14487 or SEQ ID NO: 14484 is a substitution of a threonine (T) for an alanine (A). In certain embodiments, the amino acid substitution at position 82 of SEQ ID NO: 14487 or SEQ ID NO: 14484 is a substitution of a tryptophan (W) for an isoleucine (I). In certain embodiments, the amino acid substitution at position 103 of SEQ ID NO: 14487 or SEQ ID NO: 14484 is a substitution of a proline (P) for a serine (S). In certain embodiments, the amino acid substitution at position 119 of SEQ ID NO: 14487 or SEQ ID NO: 14484 is a substitution of a proline (P) for an arginine (R). In certain embodiments, the amino acid substitution at position 125 of SEQ ID NO: 14487 or SEQ ID NO: 14484 is a substitution of an alanine (A) a cysteine (C). In certain embodiments, the amino acid substitution at position 125 of SEQ ID NO: 14487 or SEQ ID NO: 14484 is a substitution of a leucine (L) for a cysteine (C). In certain embodiments, the amino acid substitution at position 177 of SEQ ID NO: 14487 or SEQ ID NO: 14484 is a substitution of a lysine (K) for a tyrosine (Y). In certain embodiments, the amino acid substitution at position 177 of SEQ ID NO: 14487 or SEQ ID NO: 14484 is a substitution of a histidine (H) for a tyrosine (Y). In certain embodiments, the amino acid substitution at position 180 of SEQ ID NO: 14487 or SEQ ID NO: 14484 is a substitution of a leucine (L) for a phenylalanine (F). In certain embodiments, the amino acid substitution at position 180 of SEQ ID NO: 14487 or SEQ ID NO: 14484 is a substitution of an isoleucine (I) for a phenylalanine (F). In certain embodiments, the amino acid substitution at position 180 of SEQ ID NO: 14487 or SEQ ID NO: 14484 is a substitution of a valine (V) for a phenylalanine (F). In certain embodiments, the amino acid substitution at position 185 of SEQ ID NO: 14487 or SEQ ID NO: 14484 is a substitution of a leucine (L) for a methionine (M). In certain embodiments, the amino acid substitution at position 187 of SEQ ID NO: 14487 or SEQ ID NO: 14484 is a substitution of a glycine (G) for an alanine (A). In certain embodiments, the amino acid substitution at position 200 of SEQ ID NO: 14487 or SEQ ID NO: 14484 is a substitution of a tryptophan (W) for a phenylalanine (F). In certain embodiments, the amino acid substitution at position 207 of SEQ ID NO: 14487 or SEQ ID NO: 14484 is a substitution of a proline (P) for a valine (V). In certain embodiments, the amino acid substitution at position 209 of SEQ ID NO: 14487 or SEQ ID NO: 14484 is a substitution of a phenylalanine (F) for a valine (V). In certain embodiments, the amino acid substitution at position 226 of SEQ ID NO: 14487 or SEQ ID NO: 14484 is a substitution of a phenylalanine (F) for a methionine (M). In certain embodiments, the amino acid substitution at position 235 of SEQ ID NO: 14487 or SEQ ID NO: 14484 is a substitution of an arginine (R) for a leucine (L). In certain embodiments, the amino acid substitution at position 240 of SEQ ID NO: 14487 or SEQ ID NO: 14484 is a substitution of a lysine (K) for a valine (V). In certain embodiments, the amino acid substitution at position 241 of SEQ ID NO: 14487 or SEQ ID NO: 14484 is a substitution of a leucine (L) for a phenylalanine (F). In certain embodiments, the amino acid substitution at position 243 of SEQ ID NO: 14487 or SEQ ID NO: 14484 is a substitution of a lysine (K) for a proline (P). In certain embodiments, the amino acid substitution at position 258 of SEQ ID NO: 14487 or SEQ ID NO: 14484 is a substitution of a serine (S) for an asparagine (N). In certain embodiments, the amino acid substitution at position 296 of SEQ ID NO: 14487 or SEQ ID NO: 14484 is a substitution of a tryptophan (W) for a leucine (L). In certain embodiments, the amino acid substitution at position 296 of SEQ ID NO: 14487 or SEQ ID NO: 14484 is a substitution of a tyrosine (Y) for a leucine (L). In certain embodiments, the amino acid substitution at position 296 of SEQ ID NO: 14487 or SEQ ID NO: 14484 is a substitution of a phenylalanine (F) for a leucine (L). In certain embodiments, the amino acid substitution at position 298 of SEQ ID NO: 14487 or SEQ ID NO: 14484 is a substitution of a leucine (L) for a methionine (M). In certain embodiments, the amino acid substitution at position 298 of SEQ ID NO: 14487 or SEQ ID NO: 14484 is a substitution of an alanine (A) for a methionine (M). In certain embodiments, the amino acid substitution at position 298 of SEQ ID NO: 14487 or SEQ ID NO: 14484 is a substitution of a valine (V) for a methionine (M). In certain embodiments, the amino acid substitution at position 311 of SEQ ID NO: 14487 or SEQ ID NO: 14484 is a substitution of an isoleucine (I) for a proline (P). In certain embodiments, the amino acid substitution at position 311 of SEQ ID NO: 14487 or SEQ ID NO: 14484 is a substitution of a valine for a proline (P). In certain embodiments, the amino acid substitution at position 315 of SEQ ID NO: 14487 or SEQ ID NO: 14484 is a substitution of a lysine (K) for an arginine (R). In certain embodiments, the amino acid substitution at position 319 of SEQ ID NO: 14487 or SEQ ID NO: 14484 is a substitution of a glycine (G) for a threonine (T). In certain embodiments, the amino acid substitution at position 327 of SEQ ID NO: 14487 or SEQ ID NO: 14484 is a substitution of an arginine (R) for a tyrosine (Y). In certain embodiments, the amino acid substitution at position 328 of SEQ ID NO: 14487 or SEQ ID NO: 14484 is a substitution of a valine (V) for a tyrosine (Y). In certain embodiments, the amino acid substitution at position 340 of SEQ ID NO: 14487 or SEQ ID NO: 14484 is a substitution of a glycine (G) for a cysteine (C). In certain embodiments, the amino acid substitution at position 340 of SEQ ID NO: 14487 or SEQ ID NO: 14484 is a substitution of a leucine (L) for a cysteine (C). In certain embodiments, the amino acid substitution at position 421 of SEQ ID NO: 14487 or SEQ ID NO: 14484 is a substitution of a histidine (H) for the aspartic acid (D). In certain embodiments, the amino acid substitution at position 436 of SEQ ID NO: 14487 or SEQ ID NO: 14484 is a substitution of an isoleucine (I) for a valine (V). In certain embodiments, the amino acid substitution at position 456 of SEQ ID NO: 14487 or SEQ ID NO: 14484 is a substitution of a tyrosine (Y) for a methionine (M). In certain embodiments, the amino acid substitution at position 470 of SEQ ID NO: 14487 or SEQ ID NO: 14484 is a substitution of a phenylalanine (F) for a leucine (L). In certain embodiments, the amino acid substitution at position 485 of SEQ ID NO: 14487 or SEQ ID NO: 14484 is a substitution of a lysine (K) for a serine (S). In certain embodiments, the amino acid substitution at position 503 of SEQ ID NO: 14487 or SEQ ID NO: 14484 is a substitution of a leucine (L) for a methionine (M). In certain embodiments, the amino acid substitution at position 503 of SEQ ID NO: 14487 or SEQ ID NO: 14484 is a substitution of an isoleucine (I) for a methionine (M). In certain embodiments, the amino acid substitution at position 552 of SEQ ID NO: 14487 or SEQ ID NO: 14484 is a substitution of a lysine (K) for a valine (V). In certain embodiments, the amino acid substitution at position 570 of SEQ ID NO: 14487 or SEQ ID NO: 14484 is a substitution of a threonine (T) for an alanine (A). In certain embodiments, the amino acid substitution at position 591 of SEQ ID NO: 14487 or SEQ ID NO: 14484 is a substitution of a proline (P) for a glutamine (Q). In certain embodiments, the amino acid substitution at position 591 of SEQ ID NO: 14487 or SEQ ID NO: 14484 is a substitution of an arginine (R) for a glutamine (Q).

In certain embodiments of the methods of the disclosure, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at one or more of positions 103, 194, 372, 375, 450, 509 and 570 of the sequence of SEQ ID NO: 14487 or SEQ ID NO: 14484. In certain embodiments of the methods of the disclosure, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at two, three, four, five, six or more of positions 103, 194, 372, 375, 450, 509 and 570 of the sequence of SEQ ID NO: 14487 or SEQ ID NO: 14484. In certain embodiments, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at positions 103, 194, 372, 375, 450, 509 and 570 of the sequence of SEQ ID NO: 14487 or SEQ ID NO: 14484. In certain embodiments, the amino acid substitution at position 103 of SEQ ID NO: 14487 or SEQ ID NO: 14484 is a substitution of a proline (P) for a serine (S). In certain embodiments, the amino acid substitution at position 194 of SEQ ID NO: 14487 or SEQ ID NO: 14484 is a substitution of a valine (V) for a methionine (M). In certain embodiments, the amino acid substitution at position 372 of SEQ ID NO: 14487 or SEQ ID NO: 14484 is a substitution of an alanine (A) for an arginine (R). In certain embodiments, the amino acid substitution at position 375 of SEQ ID NO: 14487 or SEQ ID NO: 14484 is a substitution of an alanine (A) for a lysine (K). In certain embodiments, the amino acid substitution at position 450 of SEQ ID NO: 14487 or SEQ ID NO: 14484 is a substitution of an asparagine (N) for an aspartic acid (D). In certain embodiments, the amino acid substitution at position 509 of SEQ ID NO: 14487 or SEQ ID NO: 14484 is a substitution of a glycine (G) for a serine (S). In certain embodiments, the amino acid substitution at position 570 of SEQ ID NO: 14487 or SEQ ID NO: 14484 is a substitution of a serine (S) for an asparagine (N). In certain embodiments, the piggyBac™ transposase enzyme may comprise a substitution of a valine (V) for a methionine (M) at position 194 of SEQ ID NO: 14487. In certain embodiments, including those embodiments wherein the piggyBac™ transposase enzyme may comprise a substitution of a valine (V) for a methionine (M) at position 194 of SEQ ID NO: 14487, the piggyBac™ transposase enzyme may further comprise an amino acid substitution at positions 372, 375 and 450 of the sequence of SEQ ID NO: 14487 or SEQ ID NO: 14484. In certain embodiments, the piggyBac™ transposase enzyme may comprise a substitution of a valine (V) for a methionine (M) at position 194 of SEQ ID NO: 14487, a substitution of an alanine (A) for an arginine (R) at position 372 of SEQ ID NO: 14487, and a substitution of an alanine (A) for a lysine (K) at position 375 of SEQ ID NO: 14487. In certain embodiments, the piggyBac™ transposase enzyme may comprise a substitution of a valine (V) for a methionine (M) at position 194 of SEQ ID NO: 14487, a substitution of an alanine (A) for an arginine (R) at position 372 of SEQ ID NO: 14487, a substitution of an alanine (A) for a lysine (K) at position 375 of SEQ ID NO: 14487 and a substitution of an asparagine (N) for an aspartic acid (D) at position 450 of SEQ ID NO: 14487.

The sleeping beauty transposon is transposed into the target genome by the Sleeping Beauty transposase that recognizes ITRs, and moves the contents between the ITRs into TA chromosomal sites. In various embodiments, SB transposon-mediated gene transfer, or gene transfer using any of a number of similar transposons, may be used in the compositions and methods of the disclosure.

In certain embodiments, and, in particular, those embodiments wherein the transposon is a Sleeping Beauty transposon, the transposase is a Sleeping Beauty transposase or a hyperactive Sleeping Beauty transposase (SB100X).

In certain embodiments of the methods of the disclosure, the Sleeping Beauty transposase enzyme comprises an amino acid sequence at least 75%, 80%, 85%, 90%, 95%, 99% or any percentage in between identical to:

```
(SEQ ID NO: 14485)
    1 MGKSKEISQD LRKKIVDLHK SGSSLGAISK RLKVPRSSVQ TIVRKYKHHG TTQPSYRSGR
  61 RRVLSPRDER TLVRKVQINP RTTAKDLVKM LEETGTKVSI STVKRVLYRH NLKGRSARKK
121 PLLQNRHKKA RLRFATAHGD KDRTFWRNVL WSDETKIELF GHNDHRYVWR KKGEACKPKN
181 TIPTVKHGGG SIMLWGCFAA GGTGALHKID GIMRKENYVD ILKQHLKTSV RKLKLGRKWV
241 FQMDNDPKHT SKVVAKWLKD NKVKVLEWPS QSPDLNPIEN LWAELKKRVR ARRPTNLTQL
301 HQLCQEEWAK IHPTYCGKLV EGYPKRLTQV KQFKGNATKY.
```

In certain embodiments of the methods of the disclosure, the hyperactive Sleeping Beauty (SB100X) transposase enzyme comprises an amino acid sequence at least 75%, 80%, 85%, 90%, 95%, 99% or any percentage in between identical to:

```
(SEQ ID NO: 14486)
    1 MGKSKEISQD LRKRIVDLHK SGSSLGAISK RLAVPRSSVQ TIVRKYKHHG TTQPSYRSGR
  61 RRVLSPRDER TLVRKVQINP RTTAKDLVKM LEETGTKVSI STVKRVLYRH NLKGHSARKK
121 PLLQNRHKKA RLRFATAHGD KDRTFWRNVL WSDETKIELF GHNDHRYVWR KKGEACKPKN
181 TIPTVKHGGG SIMLWGCFAA GGTGALHKID GIMDAVQYVD ILKQHLKTSV RKLKLGRKWV
241 FQHDNDPKHT SKVVAKWLKD NKVKVLEWPS QSPDLNPIEN LWAELKKRVR ARRPTNLTQL
301 HQLCQEEWAK IHPNYCGKLV EGYPKRLTQV KQFKGNATKY.
```

The Helraiser transposon is transposed by the Helitron transposase. Helitron transposases mobilize the Helraiser transposon, an ancient element from the bat genome that was active about 30 to 36 million years ago. An exemplary Helraiser transposon of the disclosure includes Helibatl, which comprises a nucleic acid sequence comprising:

```
(SEQ ID NO: 18061)
      1 TCCTATATAA TAAAAGAGAA ACATGCAAAT TGACCATCCC TCCGCTACGC TCAAGCCACG
    61 CCCACCAGCC AATCAGAAGT GACTATGCAA ATTAACCCAA CAAAGATGGC AGTTAAATTT
  121 GCATACGCAG GTGTCAAGCG CCCCAGGAGG CAACGGCGGC CGCGGGCTCC CAGGACCTTC
  181 GCTGGCCCCG GGAGGCGAGG CCGGCCGCGC CTAGCCACAC CCGCGGGCTC CCGGGACCTT
  241 CGCCAGCAGA GAGCAGAGCG GGAGAGCGGG CGGAGAGCGG GAGGTTTGGA GGACTTGGCA
  301 GAGCAGGAGG CCGCTGGACA TAGAGCAGAG CGAGAGAGAG GGTGGCTTGG AGGGCGTGGC
  361 TCCCTCTGTC ACCCCAGCTT CCTCATCACA GCTGTGGAAA CTGACAGCAG GGAGGAGGAA
  421 GTCCCACCCC CACAGAATCA GCCAGAATCA GCCGTTGGTC AGACAGCTCT CAGCGGCCTG
  481 ACAGCCAGGA CTCTCATTCA CCTGCATCTC AGACCGTGAC AGTAGAGAGG TGGGACTATG
  541 TCTAAAGAAC AACTGTTGAT ACAACGTAGC TCTGCAGCCG AAAGATGCCG GCGTTATCGA
  601 CAGAAAATGT CTGCAGAGCA ACGTGCGTCT GATCTTGAAA GAAGGCGGCG CCTGCAACAG
  661 AATGTATCTG AAGAGCAGCT ACTGGAAAAA CGTCGCTCTG AAGCCGAAAA ACAGCGGCGT
  721 CATCGACAGA AAATGTCTAA AGACCAACGT GCCTTTGAAG TTGAAAGAAG GCGGTGGCGA
  781 CGACAGAATA TGTCTAGAGA ACAGTCATCA ACAAGTACTA CCAATACCGG TAGGAACTGC
  841 CTTCTCAGCA AAAATGGAGT ACATGAGGAT GCAATTCTCG AACATAGTTG TGGTGGAATG
  901 ACTGTTCGAT GTGAATTTTG CCTATCACTA AATTTCTCTG ATGAAAAACC ATCCGATGGG
  961 AAATTTACTC GATGTTGTAG CAAAGGGAAA GTCTGTCCAA ATGATATACA TTTTCCAGAT
1021 TACCCGGCAT ATTTAAAAAG ATTAATGACA AACGAAGATT CTGACAGTAA AAATTTCATG
1081 GAAAATATTC GTTCCATAAA TAGTTCTTTT GCTTTTGCTT CCATGGGTGC AAATATTGCA
1141 TCGCCATCAG GATATGGGCC ATACTGTTTT AGAATACACG GACAAGTTTA TCACCGTACT
1201 GGAACTTTAC ATCCTTCGGA TGGTGTTTCT CGGAAGTTTG CTCAACTCTA TATTTTGGAT
1261 ACAGCCGAAG CTACAAGTAA AAGATTAGCA ATGCCAGAAA ACCAGGGCTG CTCAGAAAGA
1321 CTCATGATCA ACATCAACAA CCTCATGCAT GAAATAAATG AATTAACAAA ATCGTACAAG
1381 ATGCTACATG AGGTAGAAAA GGAAGCCCAA TCTGAAGCAG CAGCAAAAGG TATTGCTCCC
1441 ACAGAAGTAA CAATGGCGAT TAAATACGAT CGTAACAGTG ACCCAGGTAG ATATAATTCT
1501 CCCCGTGTAA CCGAGGTTGC TGTCATATTC AGAAACGAAG ATGGAGAACC TCCTTTTGAA
1561 AGGGACTTGC TCATTCATTG TAAACCAGAT CCCAATAATC CAAATGCCAC TAAAATGAAA
1621 CAAATCAGTA TCCTGTTTCC TACATTAGAT GCAATGACAT ATCCTATTCT TTTTCCACAT
1681 GGTGAAAAAG GCTGGGGAAC AGATATTGCA TTAAGACTCA GAGACAACAG TGTAATCGAC
1741 AATAATACTA GACAAAATGT AAGGACACGA GTCACACAAA TGCAGTATTA TGGATTTCAT
1801 CTCTCTGTGC GGGACACGTT CAATCCTATT TTAAATGCAG GAAAATTAAC TCAACAGTTT
1861 ATTGTGGATT CATATTCAAA AATGGAGGCC AATCGGATAA ATTTCATCAA AGCAAACCAA
1921 TCTAAGTTGA GAGTTGAAAA ATATAGTGGT TTGATGGATT ATCTCAAATC TAGATCTGAA
1981 AATGACAATG TGCCGATTGG TAAAATGATA ATACTTCCAT CATCTTTTGA GGGTAGTCCC
2041 AGAAATATGC AGCAGCGATA TCAGGATGCT ATGGCAATTG TAACGAAGTA TGGCAAGCCC
2101 GATTTATTCA TAACCATGAC ATGCAACCCC AAATGGGCAG ATATTACAAA CAATTTACAA
2161 CGCTGGCAAA AAGTTGAAAA CAGACCTGAC TTGGTAGCCA GAGTTTTTAA TATTAAGCTG
2221 AATGCTCTTT TAAATGATAT ATGTAAATTC CATTTATTTG GCAAAGTAAT AGCTAAAATT
2281 CATGTCATTG AATTTCAGAA ACGCGGACTG CCTCACGCTC ACATATTATT GATATTAGAT
2341 AGTGAGTCCA AATTACGTTC AGAAGATGAC ATTGACCGTA TAGTTAAGGC AGAAATTCCA
2401 GATGAAGACC AGTGTCCTCG ACTTTTTCAA ATTGTAAAAT CAAATATGGT ACATGGACCA
2461 TGTGGAATAC AAAATCCAAA TAGTCCATGT ATGGAAAATG GAAAATGTTC AAAGGGATAT
2521 CCAAAAGAAT TTCAAAATGC GACCATTGGA AATATTGATG GATATCCCAA ATACAAACGA
2581 AGATCTGGTA GCACCATGTC TATTGGAAAT AAAGTTGTCG ATAACACTTG GATTGTCCCT
2641 TATAACCCGT ATTTGTGCCT TAAATATAAC TGTCATATAA ATGTTGAAGT CTGTGCATCA
2701 ATTAAAAGTG TCAAATATTT ATTTAAATAC ATCTATAAAG GGCACGATTG TGCAAATATT
2761 CAAATTTCTG AAAAAAATAT TATCAATCAT GACGAAGTAC AGGACTTCAT TGACTCCAGG
2821 TATGTGAGCG CTCCTGAGGC TGTTTGGAGA CTTTTTGCAA TGCGAATGCA TGACCAATCT
2881 CATGCAATCA CAAGATTAGC TATTCATTTG CCAAATGATC AGAATTTGTA TTTTCATACC
2941 GATGATTTTG CTGAAGTTTT AGATAGGGCT AAAAGGCATA ACTCGACTTT GATGGCTTGG
3001 TTCTTATTGA ATAGAGAAGA TTCTGATGCA CGTAATTATT ATTATTGGGA GATTCCACAG
3061 CATTATGTGT TTAATAATTC TTTGTGGACA AAACGCCGAA AGGGTGGGAA TAAAGTATTA
3121 GGTAGACTGT TCACTGTGAG CTTTAGAGAA CCAGAACGAT ATTACCTTAG ACTTTTGCTT
3181 CTGCATGTAA AAGGTGCGAT AAGTTTTGAG GATCTGCGAA CTGTAGGAGG TGTAACTTAT
3241 GATACATTTC ATGAAGCTGC TAAACACCGA GGATTATTAC TTGATGACAC TATCTGGAAA
3301 GATACGATTG ACGATGCAAT CATCCTTAAT ATGCCCAAAC AACTACGGCA ACTTTTTGCA
3361 TATATATGTG TGTTTGGATG TCCTTCTGCT GCAGACAAAT TATGGGATGA GAATAAATCT
3421 CATTTTATTG AAGATTTCTG TTGGAAATTA CACCGAAGAG AAGGTGCCTG TGTGAACTGT
3481 GAAATGCATG CCCTTAACGA AATTCAGGAG GTATTCACAT TGCATGGAAT GAAATGTTCA
3541 CATTTCAAAC TTCCGGACTA TCCTTTATTA ATGAATGCAA ATACATGTGA TCAATTGTAC
3601 GAGCAACAAC AGGCAGAGGT TTTGATAAAT TCTCTGAATG ATGAACAGTT GGCAGCCTTT
3661 CAGACTATAA CTTCAGCCAT CGAAGATCAA ACTGTACACC CCAAATGCTT TTTCTTGGAT
3721 GGTCCAGGTG GTAGTGGAAA AACATATCTG TATAAAGTTT TAACACATTA TATTAGAGGT
3781 CGTGGTGGTA CTGTTTTACC CACAGCATCT ACAGGAATTG CTGCAAATTT ACTTCTTGGT
3841 GGAAGAACCT TTCATTCCCA ATATAAATTA CCAATTCCAT TAAATGAAAC TTCAATTTCT
3901 AGACTCGATA TAAAGAGTGA AGTTGCTAAA ACCATTAAAA AGGCCCAACT TCTCATTATT
3961 GATGAATGCA CCATGGCATC CAGTCATGCT ATAAACGCCA TAGATAGATT ACTAAGAGAA
4021 ATTATGAATT TGAATGTTGC ATTTGGTGGG AAAGTTCTCC TTCTCGGAGG GGATTTTCGA
4081 CAATGTCTCA GTATTGTACC ACATGCTATG CGATCGGCCA TAGTACAAAC GAGTTTAAAG
4141 TACTGTAATG TTTGGGGATG TTTCAGAAAG TTGTCTCTTA AAACAAATAT GAGATCAGAG
4201 GATTCTGCTT ATAGTGAATG GTTAGTAAAA CTTGGAGATG GCAAACTTGA TAGCAGTTTT
4261 CATTTAGGAA TGGATATTAT TGAAATCCCC CATGAAATGA TTTGTAACGG ATCTATTATT
4321 GAAGCTACCT TTGGAAATAG TATATCTATA GATAATATTA AAAATATATC TAAACGTGCA
4381 ATTCTTTGTC CAAAAAATGA GCATGTTCAA AAATTAAATG AAGAAATTTT GGATATACTT
4441 GATGGAGATT TTCACACATA TTTGAGTGAT GATTCCATTG ATTCAACAGA TGATGCTGAA
4501 AAGGAAAATT TTCCCATCGA ATTTCTTAAT AGTATTACTC CTTCGGGAAT GCCGTGTCAT
4561 AAATTAAAAT TGAAAGTGGG TGCAATCATC ATGCTATTGA GAAATCTTAA TAGTAAATGG
4621 GGTCTTTGTA ATGGTACTAG ATTTATTATC AAAAGATTAC GACCTAACAT TATCGAAGCT
4681 GAAGTATTAA CAGGATCTGC AGAGGGAGAG GTTGTTCTGA TTCCAAGAAT TGATTTGTCC
4741 CCATCTGACA CTGGCCTCCC ATTTAAATTA ATTCGAAGAC AGTTTCCCGT GATGCCAGCA
4801 TTTGCGATGA CTATTAATAA ATCACAAGGA CAAACTCTAG ACAGAGTAGG AATATTCCTA
4861 CCTGAACCCG TTTTCGCACA TGGTCAGTTA TATGTTGCTT TCTCTCGAGT TCGAAGAGCA
4921 TGTGACGTTA AAGTTAAAGT TGTAAATACT TCATCACAAG GGAAATTAGT CAAGCACTCT
4981 GAAAGTGTTT TTACTCTTAA TGTGGTATAC AGGGAGATAT TAGAATAAGT TTAATCACTT
5041 TATCAGTCAT TGTTTGCATC AATGTTGTTT TTATATCATG TTTTTGTTGT TTTTATATCA
5101 TGTCTTTGTT GTTGTTATAT CATGTTGTTA TTGTTTATTT ATTAATAAAT TTATGTATTA
5161 TTTTCATATA CATTTTACTC ATTTCCTTTC ATCTCTCACA CTTCTATTAT AGAGAAAGGG
5221 CAAATAGCAA TATTAAAATA TTTCCTCTAA TTAATTCCCT TTCAATGTGC ACGAATTTCG
5281 TGCACCGGGC CACTAG.
```

Unlike other transposases, the Helitron transposase does not contain an RNase-H like catalytic domain, but instead comprises a RepHel motif made up of a replication initiator domain (Rep) and a DNA helicase domain. The Rep domain is a nuclease domain of the HUH superfamily of nucleases.

An exemplary Helitron transposase of the disclosure comprises an amino acid sequence comprising:

```
(SEQ ID NO: 14501)
      1 MSKEQLLIQR SSAAERCRRY RQKMSAEQRA SDLERRRRLQ QNVSEEQLLE KRRSEAEKQR
    61 RHRQKMSKDQ RAFEVERRRW RRQNMSREQS STSTTNTGRN CLLSKNGVHE DAILEHSCGG
  121 MTVRCEFCLS LNFSDEKPSD GKFTRCCSKG KVCPNDIHFP DYPAYLKRLM TNEDSDSKNF
  181 MENIRSINSS FAFASMGANI ASPSGYGPYC FRIHGQVYHR TGTLHPSDGV SRKFAQLYIL
  241 DTAEATSKRL AMPENQGCSE RLMININNLM HEINELTKSY KMLHEVEKEA QSEAAAKGIA
  301 PTEVIMAIKY DRNSDPGRYN SPRVTEVAVI FRNEDGEPPF ERDLLIHCKP DPNNPNATKM
  361 KQISILFPTL DAMTYPILFP HGEKGWGTDI ALRLRDNSVI DNNTRQNVRT RVTQMQYYGF
  421 HLSVRDTFNP ILNAGKLTQQ FIVDSYSKME ANRINFIKAN QSKLRVEKYS GLMDYLKSRS
  481 ENDNVPIGKM IILPSSFEGS PRNMQQRYQD AMAIVTKYGK PDLFITMTCN PKWADITNNL
  541 QRWQKVENRP DLVARVFNIK LNALLNDICK FHLFGKVIAK IHVIEFQKRG LPHAHILLIL
  601 DSESKLRSED DIDRIVKAEI PDEDQCPRLF QIVKSNMVHG PCGIQNPNSP CMENGKCSKG
  661 YPKEFQNATI GNIDGYPKYK RRSGSTMSIG NKVVDNTWIV PYNPYLCLKY NCHINVEVCA
  721 SIKSVKYLFK YIYKGHDCAN IQISEKNIIN HDEVQDFIDS RYVSAPEAVW RLFAMRMHDQ
  781 SHAITRLAIH LPNDQNLYFH TDDFAEVLDR AKRHNSTLMA WFLLNREDSD ARNYYYWEIP
  841 QHYVFNNSLW TKRRKGGNKV LGRLFTVSFR EPERYYLRLL LLHVKGAISF EDLRTVGGVT
  901 YDTFHEAAKH RGLLLDDTIW KDTIDDAIIL NMPKQLRQLF AYICVFGCPS AADKLWDENK
  961 SHFIEDFCWK LHRREGACVN CEMHALNEIQ EVFTLHGMKC SHFKLPDYPL LMNANTCDQL
1021 YEQQQAEVLI NSLNDEQLAA FQTITSAIED QTVHPKCFFL DGPGGSGKTY LYKVLTHYIR
1081 GRGGTVLPTA STGIAANLLL GGRTFHSQYK LPIPLNETSI SRLDIKSEVA KTIKKAQLLI
1141 IDECTMASSH AINAIDRLLR EIMNLNVAFG GKVLLLGGDF RQCLSIVPHA MRSAIVQTSL
1201 KYCNVWGCFR KLSLKTNMRS EDSAYSEWLV KLGDGKLDSS FHLGMDIIEI PHEMICNGSI
1261 IEATFGNSIS IDNIKNISKR AILCPKNEHV QKLNEEILDI LDGDFHTYLS DDSIDSTDDA
1321 EKENFPIEFL NSITPSGMPC HKLKLKVGAI IMLLRNLNSK WGLCNGTRFI IKRLRPNIIE
1381 AEVLTGSAEG EVVLIPRIDL SPSDTGLPFK LIRRQFPVMP AFAMTINKSQ GQTLDRVGIF
1441 LPEPVFAHGQ LYVAFSRVRR ACDVKVKVVN TSSQGKLVKH SESVFTLNVV YREILE.
```

In Helitron transpositions, a hairpin close to the 3′ end of the transposon functions as a terminator. However, this hairpin can be bypassed by the transposase, resulting in the transduction of flanking sequences. In addition, Helraiser transposition generates covalently closed circular intermediates. Furthermore, Helitron transpositions can lack target site duplications. In the Helraiser sequence, the transposase is flanked by 5′ and 3′ terminal sequences termed LTS and RTS. These sequences terminate with a conserved 5′-TC/CTAG-3′ motif. A 19 bp palindromic sequence with the potential to form the hairpin termination structure is located 11 nucleotides upstream of the RTS and consists of the sequence

(SEQ ID NO: 14500)
GTGCACGAATTTCGTGCACCGGGCCACTAG.

Tol2 transposons may be isolated or derived from the genome of the medaka fish, and may be similar to transposons of the hAT family. Exemplary Tol2 transposons of the disclosure are encoded by a sequence comprising about 4.7 kilobases and contain a gene encoding the Tol2 transposase, which contains four exons. An exemplary Tol2 transposase of the disclosure comprises an amino acid sequence comprising the following:

```
(SEQ ID NO: 14502)
    1 MEEVCDSSAA ASSTVQNQPQ DQEHPWPYLR EFFSLSGVNK DSFKMKCVLC LPLNKEISAF
  61 KSSPSNLRKH IERMHPNYLK NYSKLTAQKR KIGTSTHASS SKQLKVDSVF PVKHVSPVTV
121 NKAILRYIIQ GLHPFSTVDL PSFKELISTL QPGISVITRP TLRSKIAEAA LIMKQKVTAA
181 MSEVEWIATT TDCWTARRKS FIGVTAHWIN PGSLERHSAA LACKRLMGSH TFEVLASAMN
241 DIHSEYEIRD KVVCTTTDSG SNFMKAFRVF GVENNDIETE ARRCESDDTD SEGCGEGSDG
301 VEFQDASRVL DQDDGFEFQL PKHQKCACHL LNLVSSVDAQ KALSNEHYKK LYRSVFGKCQ
361 ALWNKSSRSA LAAEAVESES RLQLLRPNQT RWNSTFMAVD RILQICKEAG EGALRNICTS
421 LEVPMFNPAE MLFLTEWANT MRPVAKVLDI LQAETNTQLG WLLPSVHQLS LKLQRLHHSL
481 RYCDPLVDAL QQGIQTRFKH MFEDPEITAA AILLPKFRTS WTNDETIIKR GMDYIRVHLE
541 PLDHKKELAN SSSDDEDFFA SLKPTTHEAS KELDGYLACV SDTRESLLTF PAICSLSIKT
601 NTPLPASAAC ERLFSTAGLL FSPKRARLDT NNFENQLLLK LNLRFYNFE.
```

An exemplary Tol2 transposon of the disclosure, including inverted repeats, subterminal sequences and the Tol2 transposase, is encoded by a nucleic acid sequence comprising the following:

```
(SEQ ID NO: 18062)
      1 CAGAGGTGTA AAGTACTTGA GTAATTTTAC TTGATTACTG TACTTAAGTA TTATTTTTGG
    61 GGATTTTTAC TTTACTTGAG TACAATTAAA AATCAATACT TTTACTTTTA CTTAATTACA
  121 TTTTTTTAGA AAAAAAAGTA CTTTTTACTC CTTACAATTT TATTTACAGT CAAAAAGTAC
  181 TTATTTTTTG GAGATCACTT CATTCTATTT TCCCTTGCTA TTACCAAACC AATTGAATTG
  241 CGCTGATGCC CAGTTTAATT TAAATGTTAT TTATTCTGCC TATGAAAATC GTTTTCACAT
  301 TATATGAAAT TGGTCAGACA TGTTCATTGG TCCTTTGGAA GTGACGTCAT GTCACATCTA
  361 TTACCACAAT GCACAGCACC TTGACCTGGA AATTAGGGAA ATTATAACAG TCAATCAGTG
  421 GAAGAAAATG GAGGAAGTAT GTGATTCATC AGCAGCTGCG AGCAGCACAG TCCAAAATCA
  481 GCCACAGGAT CAAGAGCACC CGTGGCCGTA TCTTCGCGAA TTCTTTTCTT TAAGTGGTGT
  541 AAATAAAGAT TCATTCAAGA TGAAATGTGT CCTCTGTCTC CCGCTTAATA AAGAAATATC
  601 GGCCTTCAAA AGTTCGCCAT CAAACCTAAG GAAGCATATT GAGGTAAGTA CATTAAGTAT
  661 TTTGTTTTAC TGATAGTTTT TTTTTTTTTT TTTTTTTTTT TTTTTGGGTG TGCATGTTTT
  721 GACGTTGATG GCGCGCCTTT TATATGTGTA GTAGGCCTAT TTTCACTAAT GCATGCGATT
  781 GACAATATAA GGCTCACGTA ATAAAATGCT AAAATGCATT TGTAATTGGT AACGTTAGGT
  841 CCACGGGAAA TTTGGCGCCT ATTGCAGCTT TGAATAATCA TTATCATTCC GTGCTCTCAT
  901 TGTGTTTGAA TTCATGCAAA ACACAAGAAA ACCAAGCGAG AAATTTTTTT CCAAACATGT
  961 TGTATTGTCA AAACGGTAAC ACTTTACAAT GAGGTTGATT AGTTCATGTA TTAACTAACA
1021 TTAAATAACC ATGAGCAATA CATTTGTTAC TGTATCTGTT AATCTTTGTT AACGTTAGTT
1081 AATAGAAATA CAGATGTTCA TTGTTTGTTC ATGTTAGTTC ACAGTGCATT AACTAATGTT
1141 AACAAGATAT AAAGTATTAG TAAATGTTGA AATTAACATG TATACGTGCA GTTCATTATT
1201 AGTTCATGTT AACTAATGTA GTTAACTAAC GAACCTTATT GTAAAAGTGT TACCATCAAA
1261 ACTAATGTAA TGAAATCAAT TCACCCTGTC ATGTCAGCCT TACAGTCCTG TGTTTTTGTC
1321 AATATAATCA GAAATAAAAT TAATGTTTGA TTGTCACTAA ATGCTACTGT ATTTCTAAAA
1381 TCAACAAGTA TTTAACATTA TAAAGTGTGC AATTGGCTGC AAATGTCAGT TTTATTAAAG
1441 GGTTAGTTCA CCCAAAAATG AAAATAATGT CATTAATGAC TCGCCCTCAT GTCGTTCCAA
1501 GCCCGTAAGA CCTCCGTTCA TCTTCAGAAC ACAGTTTAAG ATATTTTAGA TTTAGTCCGA
1561 GAGCTTTCTG TGCCTCCATT GAGAATGTAT GTACGGTATA CTGTCCATGT CCAGAAAGGT
1621 AATAAAAACA TCAAAGTAGT CCATGTGACA TCAGTGGGTT AGTTAGAATT TTTTGAAGCA
1681 TCGAATACAT TTTGGTCCAA AAATAACAAA ACCTACGACT TTATTCGGCA TTGTATTCTC
1741 TTCCGGGTCT GTTGTCAATC CGCGTTCACG ACTTCGCAGT GACGCTACAA TGCTGAATAA
1801 AGTCGTAGGT TTTGTTATTT TTGGACCAAA ATGTATTTTC GATGCTTCAA ATAATTCTAC
1861 CTAACCCACT GATGTCACAT GGACTACTTT GATGTTTTTA TTACCTTTCT GGACATGGAC
1921 AGTATACCGT ACATACATTT TCAGTGGAGG GACAGAAAGC TCTCGGACTA AATCTAAAAT
1981 ATCTTAAACT GTGTTCCGAA GATGAACGGA GGTGTTACGG GCTTGGAACG ACATGAGGGT
2041 GAGTCATTAA TGACATCTTT TCATTTTTGG GTGAACTAAC CCTTTAATGC TGTAATCAGA
2101 GAGTGTATGT GTAATTGTTA CATTTATTGC ATACAATATA AATATTTATT TGTTGTTTTT
2161 ACAGAGAATG CACCCAAATT ACCTCAAAAA CTACTCTAAA TTGACAGCAC AGAAGAGAAA
2221 GATCGGGACC TCCACCCATG CTTCCAGCAG TAAGCAACTG AAAGTTGACT CAGTTTTCCC
2281 AGTCAAACAT GTGTCTCCAG TCACTGTGAA CAAAGCTATA TTAAGGTACA TCATTCAAGG
2341 ACTTCATCCT TTCAGCACTG TTGATCTGCC ATCATTTAAA GAGCTGATTA GTACACTGCA
2401 GCCTGGCATT TCTGTCATTA CAAGGCCTAC TTTACGCTCC AAGATAGCTG AAGCTGCTCT
2461 GATCATGAAA CAGAAAGTGA CTGCTGCCAT GAGTGAAGTT GAATGGATTG CAACCACAAC
2521 GGATTGTTGG ACTGCACGTA GAAAGTCATT CATTGGTGTA ACTGCTCACT GGATCAACCC
2581 TGGAAGTCTT GAAAGACATT CCGCTGCACT TGCCTGCAAA AGATTAATGG GCTCTCATAC
2641 TTTTGAGGTA CTGGCCAGTG CCATGAATGA TATCCACTCA GAGTATGAAA TACGTGACAA
2701 GGTTGTTTGC ACAACCACAG ACAGTGGTTC CAACTTTATG AAGGCTTTCA GAGTTTTTGG
2761 TGTGGAAAAC AATGATATCG AGACTGAGGC AAGAAGGTGT GAAAGTGATG ACACTGATTC
2821 TGAAGGCTGT GGTGAGGGAA GTGATGGTGT GGAATTCCAA GATGCCTCAC GAGTCCTGGA
2881 CCAAGACGAT GGCTTCGAAT TCCAGCTACC AAAACATCAA AAGTGTGCCT GTCACTTACT
2941 TAACCTAGTC TCAAGCGTTG ATGCCCAAAA AGCTCTCTCA AATGAACACT ACAAGAAACT
3001 CTACAGATCT GTCTTTGGCA AATGCCAAGC TTTATGGAAT AAAAGCAGCC GATCGGCTCT
3061 AGCAGCTGAA GCTGTTGAAT CAGAAAGCCG GCTTCAGCTT TTAAGGCCAA ACCAAACGCG
3121 GTGGAATTCA ACTTTTATGG CTGTTGACAG AATTCTTCAA ATTTGCAAAG AAGCAGGAGA
3181 AGGCGCACTT CGGAATATAT GCACCTCTCT TGAGGTTCCA ATGTAAGTGT TTTTCCCCTC
3241 TATCGATGTA AACAAATGTG GGTTGTTTTT GTTTAATACT CTTTGATTAT GCTGATTTCT
3301 CCTGTAGGTT TAATCCAGCA GAAATGCTGT TCTTGACAGA GTGGGCCAAC ACAATGCGTC
3361 CAGTTGCAAA AGTACTCGAC ATCTTGCAAG CGGAAACGAA TACACAGCTG GGGTGGCTGC
3421 TGCCTAGTGT CCATCAGTTA AGCTTGAAAC TTCAGCGACT CCACCATTCT CTCAGGTACT
3481 GTGACCCACT TGTGGATGCC CTACAACAAG GAATCCAAAC ACGATTCAAG CATATGTTTG
3541 AAGATCCTGA GATCATAGCA GCTGCCATCC TTCTCCCTAA ATTTCGGACC TCTTGGACAA
3601 ATGATGAAAC CATCATAAAA CGAGGTAAAT GAATGCAAGC AACATACACT TGACGAATTC
3661 TAATCTGGGC AACCTTTGAG CCATACCAAA ATTATTCTTT TATTTATTTA TTTTTGCACT
3721 TTTTAGGAAT GTTATATCCC ATCTTTGGCT GTGATCTCAA TATGAATATT GATGTAAAGT
3781 ATTCTTGCAG CAGGTTGTAG TTATCCCTCA GTGTTTCTTG AAACCAAACT CATATGTATC
3841 ATATGTGGTT TGGAAATGCA GTTAGATTTT ATGCTAAAAT AAGGGATTTG CATGATTTTA
3901 GATGTAGATG ACTGCACGTA AATGTAGTTA ATGACAAAAT CCATAAAATT TGTTCCCAGT
3961 CAGAAGCCCC TCAACCAAAC TTTTCTTTGT GTCTGCTCAC TGTGCTTGTA GGCATGGACT
4021 ACATCAGAGT GCATCTGGAG CCTTTGGACC ACAAGAAGGA ATTGGCCAAC AGTTCATCTG
4081 ATGATGAAGA TTTTTTCGCT TCTTTGAAAC CGACAACACA TGAAGCCAGC AAAGAGTTGG
4141 ATGGATATCT GGCCTGTGTT TCAGACACCA GGGAGTCTCT GCTCACGTTT CCTGCTATTT
4201 GCAGCCTCTC TATCAAGACT AATACACCTC TTCCCGCATC GGCTGCCTGT GAGAGGCTTT
4261 TCAGCACTGC AGGATTGCTT TTCAGCCCCA AAAGAGCTAG GCTTGACACT AACAATTTTG
4321 AGAATCAGCT TCTACTGAAG TTAAATCTGA GGTTTTACAA CTTTGAGTAG CGTGTACTGG
4381 CATTAGATTG TCTGTCTTAT AGTTTGATAA TTAAATACAA ACAGTTCTAA AGCAGGATAA
4441 AACCTTGTAT GCATTTCATT TAATGTTTTT TGAGATTAAA AGCTTAAACA AGAATCTCTA
4501 GTTTTCTTTC TTGCTTTTAC TTTTACTTCC TTAATACTCA AGTACAATTT TAATGGAGTA
4561 CTTTTTTACT TTTACTCAAG TAAGATTCTA GCCAGATACT TTTACTTTTA ATTGAGTAAA
4621 ATTTTCCCTA AGTACTTGTA CTTTCACTTG AGTAAAATTT TTGAGTACTT TTTACACCTC
4681 TG.
```

Exemplary transposon/transposase systems of the disclosure include, but are not limited to, piggyBac and piggyBac-like transposons and transposases.

PiggyBac and piggyBac-like transposases recognizes transposon-specific inverted terminal repeat sequences (ITRs) on the ends of the transposon, and moves the contents between the ITRs into TTAA or TTAT chromosomal sites. The piggyBac or piggyBac-like transposon system has no payload limit for the genes of interest that can be included between the ITRs.

In certain embodiments, and, in particular, those embodiments wherein the transposon is a piggyBac transposon, the transposase is a piggyBac™, Super piggyBac™ (SPB) transposase. In certain embodiments, and, in particular, those embodiments wherein the transposase is a piggyBac™, Super piggyBac™ (SPB), the sequence encoding the transposase is an mRNA sequence.

In certain embodiments of the methods of the disclosure, the transposase enzyme is a piggyBac or piggyBac-like transposase enzyme.

In certain embodiments of the methods of the disclosure, the transposase enzyme is a piggyBac or a piggyBac-like transposase enzyme. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14487)
    1 MGSSLDDEHI LSALLQSDDE LVGEDSDSEI SDHVSEDDVQ SDTEEAFIDE VHEVQPTSSG
  61 SEILDEQNVI EQPGSSLASN RILTLPQRTI RGKNKHCWST SKSTRRSRVS ALNIVRSQRG
121 PTRMCRNIYD PLLCFKLFFT DEIISEIVKW TNAEISLKRR ESMTGATFRD TNEDEIYAFF
181 GILVMTAVRK DNHMSTDDLF DRSLSMVYVS VMSRDRFDFL IRCLRMDDKS IRPTLRENDV
241 FTPVRKIWDL FIHQCIQNYT PGAHLTIDEQ LLGFRGRCPF RMYIPNKPSK YGIKILMMCD
301 SGTKYMINGM PYLGRGTQTN GVPLGEYYVK ELSKPVHGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDTLD QMCSVMTCSR KTNRWPMALL YGMINIACIN
481 SFIIYSHNVS SKGEKVQSRK KFMRNLYMSL TSSFMRKRLE APTLKRYLRD NISNILPNEV
541 PGTSDDSTEE PVMKKRTYCT YCPSKIRRKA NASCKKCKKV ICREHNIDMC QSCF.
```

In certain embodiments of the methods of the disclosure, the transposase enzyme is a piggyBac or piggyBac-like transposase enzyme that comprises or consists of an amino acid sequence having an amino acid substitution at one or more of positions 30, 165, 282, or 538 of the sequence:

```
(SEQ ID NO: 14487)
    1 MGSSLDDEHI LSALLQSDDE LVGEDSDSEI SDHVSEDDVQ SDTEEAFIDE VHEVQPTSSG
  61 SEILDEQNVI EQPGSSLASN RILTLPQRTI RGKNKHCWST SKSTRRSRVS ALNIVRSQRG
121 PTRMCRNIYD PLLCFKLFFT DEIISEIVKW TNAEISLKRR ESMTGATFRD TNEDEIYAFF
181 GILVMTAVRK DNHMSTDDLF DRSLSMVYVS VMSRDRFDFL IRCLRMDDKS IRPTLRENDV
241 FTPVRKIWDL FIHQCIQNYT PGAHLTIDEQ LLGFRGRCPF RMYIPNKPSK YGIKILMMCD
301 SGTKYMINGM PYLGRGTQTN GVPLGEYYVK ELSKPVHGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDTLD QMCSVMTCSR KTNRWPMALL YGMINIACIN
481 SFIIYSHNVS SKGEKVQSRK KFMRNLYMSL TSSFMRKRLE APTLKRYLRD NISNILPNEV
541 PGTSDDSTEE PVMKKRTYCT YCPSKIRRKA NASCKKCKKV ICREHNIDMC QSCF.
```

In certain embodiments, the transposase enzyme is a piggyBac or piggyBac-like transposase enzyme that comprises or consists of an amino acid sequence having an amino acid substitution at two or more of positions 30, 165, 282, or 538 of the sequence of SEQ ID NO: 14487. In certain embodiments, the transposase enzyme is a piggyBac or piggyBac-like transposase enzyme that comprises or consists of an amino acid sequence having an amino acid substitution at three or more of positions 30, 165, 282, or 538 of the sequence of SEQ ID NO: 14487. In certain embodiments, the transposase enzyme is a piggyBac or piggyBac-like transposase enzyme that comprises or consists of an amino acid sequence having an amino acid substitution at each of the following positions 30, 165, 282, and 538 of the sequence of SEQ ID NO: 14487. In certain embodiments, the amino acid substitution at position 30 of the sequence of SEQ ID NO: 14487 is a substitution of a valine (V) for an isoleucine (I). In certain embodiments, the amino acid substitution at position 165 of the sequence of SEQ ID NO: 14487 is a substitution of a serine (S) for a glycine (G). In certain embodiments, the amino acid substitution at position 282 of the sequence of SEQ ID NO: 14487 is a substitution of a valine (V) for a methionine (M). In certain embodiments, the amino acid substitution at position 538 of the sequence of SEQ ID NO: 14487 is a substitution of a lysine (K) for an asparagine (N).

In certain embodiments of the methods of the disclosure, the transposase enzyme is a Super piggyBac™ (SPB) or piggyBac-like transposase enzyme. In certain embodiments, the Super piggyBac™ (SPB) or piggyBac-like transposase enzyme of the disclosure may comprise or consist of the amino acid sequence of the sequence of SEQ ID NO: 14487 wherein the amino acid substitution at position 30 is a substitution of a valine (V) for an isoleucine (I), the amino acid substitution at position 165 is a substitution of a serine (S) for a glycine (G), the amino acid substitution at position 282 is a substitution of a valine (V) for a methionine (M), and the amino acid substitution at position 538 is a substitution of a lysine (K) for an asparagine (N). In certain embodiments, the Super piggyBac™ (SPB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14484)
    1 MGSSLDDEHI LSALLQSDDE LVGEDSDSEV SDHVSEDDVQ SDTEEAFIDE VHEVQPTSSG
  61 SEILDEQNVI EQPGSSLASN RILTLPQRTI RGKNKHCWST SKSTRRSRVS ALNIVRSQRG
121 PTRMCRNIYD PLLCFKLFFT DEIISEIVKW TNAEISLKRR ESMTSATFRD TNEDEIYAFF
181 GILVMTAVRK DNHMSTDDLF DRSLSMVYVS VMSRDRFDFL IRCLRMDDKS IRPTLRENDV
241 FTPVRKIWDL FIHQCIQNYT PGAHLTIDEQ LLGFRGRCPF RVYIPNKPSK YGIKILMMCD
301 SGTKYMINGM PYLGRGTQTN GVPLGEYYVK ELSKPVHGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDTLD QMCSVMTCSR KTNRWPMALL YGMINIACIN
481 SFIIYSHNVS SKGEKVQSRK KFMRNLYMSL TSSFMRKRLE APTLKRYLRD NISNILPKEV
541 PGTSDDSTEE PVMKKRTYCT YCPSKIRRKA NASCKKCKKV ICREHNIDMC QSCF.
```

In certain embodiments of the methods of the disclosure, including those embodiments wherein the transposase comprises the above-described mutations at positions 30, 165, 282 and/or 538, the piggyBac™, Super piggyBac™ or piggyBac-like transposase enzyme may further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 14487 or SEQ ID NO: 14484. In certain embodiments, including those embodiments wherein the transposase comprises the above-described mutations at positions 30, 165, 282 and/or 538, the piggyBac™, Super piggyBac™ or piggyBac-like transposase enzyme may further comprise an amino acid substitution at one or more of positions 46, 119, 125, 177, 180, 185, 187, 200, 207, 209, 226, 235, 240, 241, 243, 296, 298, 311, 315, 319, 327, 328, 340, 421, 436, 456, 470, 485, 503, 552 and 570. In certain embodiments, the amino acid substitution at position 3 of SEQ ID NO: 14487 or SEQ ID NO: 14484 is a substitution of an asparagine (N) for a serine (S). In certain embodiments, the amino acid substitution at position 46 of SEQ ID NO: 14487 or SEQ ID NO: 14484 is a substitution of a serine (S) for an alanine (A). In certain embodiments, the amino acid substitution at position 46 of SEQ ID NO: 14487 or SEQ ID NO: 14484 is a substitution of a threonine (T) for an alanine (A). In certain embodiments, the amino acid substitution at position 82 of SEQ ID NO: 14487 or SEQ ID NO: 14484 is a substitution of a tryptophan (W) for an isoleucine (I). In certain embodiments, the amino acid substitution at position 103 of SEQ ID NO: 14487 or SEQ ID NO: 14484 is a substitution of a proline (P) for a serine (S). In certain embodiments, the amino acid substitution at position 119 of SEQ ID NO: 14487 or SEQ ID NO: 14484 is a substitution of a proline (P) for an arginine (R). In certain embodiments, the amino acid substitution at position 125 of SEQ ID NO: 14487 or SEQ ID NO: 14484 is a substitution of an alanine (A) a cysteine (C). In certain embodiments, the amino acid substitution at position 125 of SEQ ID NO: 14487 or SEQ ID NO: 14484 is a substitution of a leucine (L) for a cysteine (C). In certain embodiments, the amino acid substitution at position 177 of SEQ ID NO: 14487 or SEQ ID NO: 14484 is a substitution of a lysine (K) for a tyrosine (Y). In certain embodiments, the amino acid substitution at position 177 of SEQ ID NO: 14487 or SEQ ID NO: 14484 is a substitution of a histidine (H) for a tyrosine (Y). In certain embodiments, the amino acid substitution at position 180 of SEQ ID NO: 14487 or SEQ ID NO: 14484 is a substitution of a leucine (L) for a phenylalanine (F). In certain embodiments, the amino acid substitution at position 180 of SEQ ID NO: 14487 or SEQ ID NO: 14484 is a substitution of an isoleucine (I) for a phenylalanine (F). In certain embodiments, the amino acid substitution at position 180 of SEQ ID NO: 14487 or SEQ ID NO: 14484 is a substitution of a valine (V) for a phenylalanine (F). In certain embodiments, the amino acid substitution at position 185 of SEQ ID NO: 14487 or SEQ ID NO: 14484 is a substitution of a leucine (L) for a methionine (M). In certain embodiments, the amino acid substitution at position 187 of SEQ ID NO: 14487 or SEQ ID NO: 14484 is a substitution of a glycine (G) for an alanine (A). In certain embodiments, the amino acid substitution at position 200 of SEQ ID NO: 14487 or SEQ ID NO: 14484 is a substitution of a tryptophan (W) for a phenylalanine (F). In certain embodiments, the amino acid substitution at position 207 of SEQ ID NO: 14487 or SEQ ID NO: 14484 is a substitution of a proline (P) for a valine (V). In certain embodiments, the amino acid substitution at position 209 of SEQ ID NO: 14487 or SEQ ID NO: 14484 is a substitution of a phenylalanine (F) for a valine (V). In certain embodiments, the amino acid substitution at position 226 of SEQ ID NO: 14487 or SEQ ID NO: 14484 is a substitution of a phenylalanine (F) for a methionine (M). In certain embodiments, the amino acid substitution at position 235 of SEQ ID NO: 14487 or SEQ ID NO: 14484 is a substitution of an arginine (R) for a leucine (L). In certain embodiments, the amino acid substitution at position 240 of SEQ ID NO: 14487 or SEQ ID NO: 14484 is a substitution of a lysine (K) for a valine (V). In certain embodiments, the amino acid substitution at position 241 of SEQ ID NO: 14487 or SEQ ID NO: 14484 is a substitution of a leucine (L) for a phenylalanine (F). In certain embodiments, the amino acid substitution at position 243 of SEQ ID NO: 14487 or SEQ ID NO: 14484 is a substitution of a lysine (K) for a proline (P). In certain embodiments, the amino acid substitution at position 258 of SEQ ID NO: 14487 or SEQ ID NO: 14484 is a substitution of a serine (S) for an asparagine (N). In certain embodiments, the amino acid substitution at position 296 of SEQ ID NO: 14487 or SEQ ID NO: 14484 is a substitution of a tryptophan (W) for a leucine (L). In certain embodiments, the amino acid substitution at position 296 of SEQ ID NO: 14487 or SEQ ID NO: 14484 is a substitution of a tyrosine (Y) for a leucine (L). In certain embodiments, the amino acid substitution at position 296 of SEQ ID NO: 14487 or SEQ ID NO: 14484 is a substitution of a phenylalanine (F) for a leucine (L). In certain embodiments, the amino acid substitution at position 298 of SEQ ID NO: 14487 or SEQ ID NO: 14484 is a substitution of a leucine (L) for a methionine (M). In certain embodiments, the amino acid substitution at position 298 of SEQ ID NO: 14487 or SEQ ID NO: 14484 is a substitution of an alanine (A) for a methionine (M). In certain embodiments, the amino acid substitution at position 298 of SEQ ID NO: 14487 or SEQ ID NO: 14484 is a substitution of a valine (V) for a methionine (M). In certain embodiments, the amino acid substitution at position 311 of SEQ ID NO: 14487 or SEQ ID NO: 14484 is a substitution of an isoleucine (I) for a proline (P). In certain embodiments, the amino acid substitution at position 311 of SEQ ID NO: 14487 or SEQ ID NO: 14484 is a substitution of a valine for a proline (P). In certain embodiments, the amino acid substitution at position 315 of SEQ ID NO: 14487 or SEQ ID NO: 14484 is a substitution of a lysine (K) for an arginine (R). In certain embodiments, the amino acid substitution at position 319 of SEQ ID NO: 14487 or SEQ ID NO: 14484 is a substitution of a glycine (G) for a threonine (T). In certain embodiments, the amino acid substitution at position 327 of SEQ ID NO: 14487 or SEQ ID NO: 14484 is a substitution of an arginine (R) for a tyrosine (Y). In certain embodiments, the amino acid substitution at position 328 of SEQ ID NO: 14487 or SEQ ID NO: 14484 is a substitution of a valine (V) for a tyrosine (Y). In certain embodiments, the amino acid substitution at position 340 of SEQ ID NO: 14487 or SEQ ID NO: 14484 is a substitution of a glycine (G) for a cysteine (C). In certain embodiments, the amino acid substitution at position 340 of SEQ ID NO: 14487 or SEQ ID NO: 14484 is a substitution of a leucine (L) for a cysteine (C). In certain embodiments, the amino acid substitution at position 421 of SEQ ID NO: 14487 or SEQ ID NO: 14484 is a substitution of a histidine (H) for the aspartic acid (D). In certain embodiments, the amino acid substitution at position 436 of SEQ ID NO: 14487 or SEQ ID NO: 14484 is a substitution of an isoleucine (I) for a valine (V). In certain embodiments, the amino acid substitution at position 456 of SEQ ID NO: 14487 or SEQ ID NO: 14484 is a substitution of a tyrosine (Y) for a methionine (M). In certain embodiments, the amino acid substitution at position 470 of SEQ ID NO: 14487 or SEQ ID NO: 14484 is a substitution of a phenylalanine (F) for a leucine (L). In certain embodiments, the amino acid substitution at position 485 of SEQ ID NO: 14487 or SEQ ID NO: 14484 is a substitution of a lysine (K) for a serine (S). In certain embodiments, the amino acid substitution at position 503 of SEQ ID NO: 14487 or SEQ ID NO: 14484 is a substitution of a leucine (L) for a methionine (M). In certain embodiments, the amino acid substitution at position 503 of SEQ ID NO: 14487 or SEQ ID NO: 14484 is a substitution of an isoleucine (I) for a methionine (M). In certain embodiments, the amino acid substitution at position 552 of SEQ ID NO: 14487 or SEQ ID NO: 14484 is a substitution of a lysine (K) for a valine (V). In certain embodiments, the amino acid substitution at position 570 of SEQ ID NO: 14487 or SEQ ID NO: 14484 is a substitution of a threonine (T) for an alanine (A). In certain embodiments, the amino acid substitution at position 591 of SEQ ID NO: 14487 or SEQ ID NO: 14484 is a substitution of a proline (P) for a glutamine (Q). In certain embodiments, the amino acid substitution at position 591 of SEQ ID NO: 14487 or SEQ ID NO: 14484 is a substitution of an arginine (R) for a glutamine (Q).

In certain embodiments of the methods of the disclosure, including those embodiments wherein the transposase comprises the above-described mutations at positions 30, 165, 282 and/or 538, the piggyBac™ or piggyBac-like transposase enzyme or may comprise or the Super piggyBac™ transposase enzyme may further comprise an amino acid substitution at one or more of positions 103, 194, 372, 375, 450, 509 and 570 of the sequence of SEQ ID NO: 14487 or SEQ ID NO: 14484. In certain embodiments of the methods of the disclosure, including those embodiments wherein the transposase comprises the above-described mutations at positions 30, 165, 282 and/or 538, the piggyBac™ or piggyBac-like transposase enzyme may comprise or the Super piggyBac™ transposase enzyme may further comprise an amino acid substitution at two, three, four, five, six or more of positions 103, 194, 372, 375, 450, 509 and 570 of the sequence of SEQ ID NO: 14487 or SEQ ID NO: 14484. In certain embodiments, including those embodiments wherein the transposase comprises the above-described mutations at positions 30, 165, 282 and/or 538, the piggyBac™ or piggyBac-like transposase enzyme may comprise or the Super piggyBac™ transposase enzyme may further comprise an amino acid substitution at positions 103, 194, 372, 375, 450, 509 and 570 of the sequence of SEQ ID NO: 14487 or SEQ ID NO: 14484. In certain embodiments, the amino acid substitution at position 103 of SEQ ID NO: 14487 or SEQ ID NO: 14484 is a substitution of a proline (P) for a serine (S). In certain embodiments, the amino acid substitution at position 194 of SEQ ID NO: 14487 or SEQ ID NO: 14484 is a substitution of a valine (V) for a methionine (M). In certain embodiments, the amino acid substitution at position 372 of SEQ ID NO: 14487 or SEQ ID NO: 14484 is a substitution of an alanine (A) for an arginine (R). In certain embodiments, the amino acid substitution at position 375 of SEQ ID NO: 14487 or SEQ ID NO: 14484 is a substitution of an alanine (A) for a lysine (K). In certain embodiments, the amino acid substitution at position 450 of SEQ ID NO: 14487 or SEQ ID NO: 14484 is a substitution of an asparagine (N) for an aspartic acid (D). In certain embodiments, the amino acid substitution at position 509 of SEQ ID NO: 14487 or SEQ ID NO: 14484 is a substitution of a glycine (G) for a serine (S). In certain embodiments, the amino acid substitution at position 570 of SEQ ID NO: 14487 or SEQ ID NO: 14484 is a substitution of a serine (S) for an asparagine (N). In certain embodiments, the piggyBac™ or piggyBac-like transposase enzyme may comprise a substitution of a valine (V) for a methionine (M) at position 194 of SEQ ID NO: 14487. In certain embodiments, including those embodiments wherein the piggyBac™ or piggyBac-like transposase enzyme may comprise a substitution of a valine (V) for a methionine (M) at position 194 of SEQ ID NO: 14487, the piggyBac™ or piggyBac-like transposase enzyme may further comprise an amino acid substitution at positions 372, 375 and 450 of the sequence of SEQ ID NO: 14487 or SEQ ID NO: 14484. In certain embodiments, the piggyBac™ or piggyBac-like transposase enzyme may comprise a substitution of a valine (V) for a methionine (M) at position 194 of SEQ ID NO: 14487, a substitution of an alanine (A) for an arginine (R) at position 372 of SEQ ID NO: 14487, and a substitution of an alanine (A) for a lysine (K) at position 375 of SEQ ID NO: 14487. In certain embodiments, the piggyBac™ or piggyBac-like transposase enzyme may comprise a substitution of a valine (V) for a methionine (M) at position 194 of SEQ ID NO: 14487, a substitution of an alanine (A) for an arginine (R) at position 372 of SEQ ID NO: 14487, a substitution of an alanine (A) for a lysine (K) at position 375 of SEQ ID NO: 14487 and a substitution of an asparagine (N) for an aspartic acid (D) at position 450 of SEQ ID NO: 14487.

In certain embodiments, the piggyBac or piggyBac-like transposase enzyme is isolated or derived from an insect. In certain embodiments, the insect is Trichoplusia ni (GenBank Accession No. AAA87375; SEQ ID NO: 16796), Argyrogramma agnata (GenBank Accession No. GU477713; SEQ ID NO: 14534, SEQ ID NO: 16797), Anopheles gambiae (GenBank Accession No. XP_312615 (SEQ ID NO: 16798); GenBank Accession No. XP_320414 (SEQ ID NO: 16799); GenBank Accession No. XP_310729 (SEQ ID NO: 16800)), Aphis gossypii (GenBank Accession No. GU329918; SEQ ID NO: 16801, SEQ ID NO: 16802), Acyrthosiphon pisum (GenBank Accession No. XP_001948139; SEQ ID NO: 16803), Agrotis Ipsilon (GenBank Accession No. GU477714; SEQ ID NO: 14537, SEQ ID NO: 16804), Bombyx mori (GenBank Accession No. BAD11135; SEQ ID NO: 14505), Chilo suppressalis (GenBank Accession No. JX294476; SEQ ID NO: 16805, SEQ ID NO: 16806), Drosophila melanogaster (GenBank Accession No. AAL39784; SEQ ID NO: 16807), Helicoverpa armigera (GenBank Accession No. ABS18391; SEQ ID NO: 14525), Heliothis virescens (GenBank Accession No. ABD76335; SEQ ID NO: 16808), Macdunnoughia crassisigna (GenBank Accession No. EU287451; SEQ ID NO: 16809, SEQ ID NO: 16810), Pectinophora gossypiella (GenBank Accession No. GU270322; SEQ ID NO: 14530, SEQ ID NO: 16811), Tribolium castaneum (GenBank Accession No. XP_001814566; SEQ ID NO: 16812), Ctenoplusia agnata (also called Argyrogramma agnata), Messour bouvieri, Megachile rotundata, Bombus impatiens, Mamestra brassicae, Mayetiola destructor or Apis mellifera.

In certain embodiments, the piggyBac or piggyBac-like transposase enzyme is isolated or derived from an insect. In certain embodiments, the insect is Trichoplusia ni (AAA87375).

In certain embodiments, the piggyBac or piggyBac-like transposase enzyme is isolated or derived from an insect. In certain embodiments, the insect is Bombyx mori (BAD11135).

In certain embodiments, the piggyBac or piggyBac-like transposase enzyme is isolated or derived from a crustacean. In certain embodiments, the crustacean is Daphnia pulicaria (AAM76342, SEQ ID NO: 16813).

In certain embodiments, the piggyBac or piggyBac-like transposase enzyme is isolated or derived from a vertebrate. In certain embodiments, the vertebrate is Xenopus tropicalis (GenBank Accession No. BAF82026; SEQ ID NO: 14518), Homo sapiens (GenBank Accession No. NP_689808; SEQ ID NO: 16814), Mus musculus (GenBank Accession No. NP_741958; SEQ ID NO: 16815), Macaca fascicularis (GenBank Accession No. AB179012; SEQ ID NO: 16816, SEQ ID NO: 16817), Rattus norvegicus (GenBank Accession No. XP_220453; SEQ ID NO: 16818) or Myotis lucifugus.

In certain embodiments, the piggyBac or piggyBac-like transposase enzyme is isolated or derived from a urochordate. In certain embodiments, the urochordate is Ciona intestinalis (GenBank Accession No. XP_002123602; SEQ ID NO: 16819).

In certain embodiments, the piggyBac or piggyBac-like transposase inserts a transposon at the sequence 5′-TTAT-3′ within a chromosomal site (a TTAT target sequence).

In certain embodiments, the piggyBac or piggyBac-like transposase inserts a transposon at the sequence 5′-TTAA-3′ within a chromosomal site (a TTAA target sequence).

In certain embodiments, the target sequence of the piggyBac or piggyBac-like transposon comprises or consists of 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′.

In certain embodiments of the methods of the disclosure, the transposase enzyme is a piggyBac or piggyBac-like transposase enzyme. In certain embodiments, the piggyBac or piggyBac-like transposase enzyme is isolated or derived from Bombyx mori. The piggyBac or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14504)
    1 MDIERQEERI RAMLEEELSD YSDESSSEDE TDHCSEHEVN YDTEEERIDS VDVPSNSRQE
  61 EANAIIANES DSDPDDDLPL SLVRQRASAS RQVSGPFYTS KDGTKWYKNC QRPNVRLRSE
121 NIVTEQAQVK NIARDASTEY ECWNIFVTSD MLQEILTHTN SSIRHRQTKT AAENSSAETS
181 FYMQETTLCE LKALIALLYL AGLIKSNRQS LKDLWRTDGT GVDIFRTTMS LQRFQFLQNN
241 IRFDDKSTRD ERKQTDNMAA FRSIFDQFVQ CCQNAYSPSE FLTIDEMLLS FRGRCLFRVY
301 IPNKPAKYGI KILALVDAKN FDVVNLEVYA GKQPSGPYAV SNRPFEVVER LIQPVARSHR
361 NVTFDNWFTG YELMLHLLNE YRLTSVGTVR KNKRQIPESF IRTDRQPNSS VFGFQKDITL
421 VSYAPKKNKV VVVMSTMHHD NSIDESTGEK QKPEMITFYN STKAGVDVVD ELSANYNVSR
481 NSKRWPMTLF YGVLNMAAIN ACIIYRANKN VTIKRTEFIR SLGLSMIYEH LHSRNKKKNI
541 PTYLRQRIEK QLGEPSPRHV NVPGRYVRCQ DCPYKKDRKT KHSCNACAKP ICMEHAKFLC
601 ENCAELDSSL.
```

The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14505)
    1 MDIERQEERI RAMLEEELSD YSDESSSEDE TDHCSEHEVN YDTEEERIDS VDVPSNSRQE
  61 EANAIIANES DSDPDDDLPL SLVRQRASAS RQVSGPFYTS KDGTKWYKNC QRPNVRLRSE
121 NIVTEQAQVK NIARDASTEY ECWNIFVTSD MLQEILTHTN SSIRHRQTKT AAENSSAETS
181 FYMQETTLCE LKALIALLYL AGLIKSNRQS LKDLWRTDGT GVDIFRTTMS LQRFQFLQNN
241 IRFDDKSTRD ERKQTDNMAA FRSIFDQFVQ CCQNAYSPSE FLTIDEMLLS FRGRCLFRVY
301 IPNKPAKYGI KILALVDAKN FYVVNLEVYA GKQPSGPYAV SNRPFEVVER LIQPVARSHR
361 NVTFDNWFTG YELMLHLLNE YRLTSVGTVR KNKRQIPESF IRTDRQPNSS VFGFQKDITL
421 VSYAPKKNKV VVVMSTMHHD NSIDESTGEK QKPEMITFYN STKAGVDVVD ELCANYNVSR
481 NSKRWPMTLF YGVLNMAAIN ACIIYRTNKN VTIKRTEFIR SLGLSMIYEH LHSRNKKKNI
541 PTYLRQRIEK QLGEPSPRHV NVPGRYVRCQ DCPYKKDRKT KRSCNACAKP ICMEHAKFLC
601 ENCAELDSSL.
```

In certain embodiments, the piggyBac or piggyBac-like transposase is fused to a nuclear localization signal. In certain embodiments, the amino acid sequence of the piggyBac or piggyBac-like transposase fused to a nuclear localization signal is encoded by a polynucleotide sequence comprising:

```
(SEQ ID NO: 14629)
      1 atggcaccca aaaagaaacg taaagtgatg gacattgaaa gacaggaaga aagaatcagg
    61 gcgatgctcg aagaagaact gagcgactac tccgacgaat cgtcatcaga ggatgaaacc
  121 gaccactgta gcgagcatga ggttaactac gacaccgagg aggagagaat cgactctgtg
  181 gatgtgccct ccaactcacg ccaagaagag gccaatgcaa ttatcgcaaa cgaatcggac
  241 agcgatccag acgatgatct gccactgtcc ctcgtgcgcc agcgggccag cgcttcgaga
  301 caagtgtcag gtccattcta cacttcgaag gacggcacta agtggtacaa gaattgccag
  361 cgacctaacg tcagactccg ctccgagaat atcgtgaccg aacaggctca ggtcaagaat
  421 atcgcccgcg acgcctcgac tgagtacgag tgttggaata tcttcgtgac ttcggacatg
  481 ctgcaagaaa ttctgacgca caccaacagc tcgattaggc atcgccagac caagactgca
  541 gcggagaact catcggccga aacctccttc tatatgcaag agactactct gtgcgaactg
  601 aaggcgctga ttgcactgct gtacttggcc ggcctcatca aatcaaatag gcagagcctc
  661 aaagatctct ggagaacgga tggaactgga gtggatatct ttcggacgac tatgagcttg
  721 cagcggttcc agtttctgca aaacaatatc agattcgacg acaagtccac ccgggacgaa
  781 aggaaacaga ctgacaacat ggctgcgttc cggtcaatat tcgatcagtt tgtgcagtgc
  841 tgccaaaacg cttatagccc atcggaattc ctgaccatcg acgaaatgct tctctccttc
  901 cgggggcgct gcctgttccg agtgtacatc ccgaacaagc cggctaaata cggaatcaaa
  961 atcctggccc tggtggacgc caagaatttc tacgtcgtga atctcgaagt gtacgcagga
1021 aagcaaccgt cgggaccgta cgctgtttcg aaccgcccgt ttgaagtcgt cgagcggctt
1081 attcagccgg tggccagatc ccaccgcaat gttaccttcg acaattggtt caccggctac
1141 gagctgatgc ttcaccttct gaacgagtac cggctcacta gcgtggggac tgtcaggaag
1201 aacaagcggc agatcccaga atccttcatc cgcaccgacc gccagcctaa ctcgtccgtg
1261 ttcggatttc aaaaggatat cacgcttgtc tcgtacgccc ccaagaaaaa caaggtcgtg
1321 gtcgtgatga gcaccatgca tcacgacaac agcatcgacg agtcaaccgg agaaaagcaa
1381 aagcccgaga tgatcacctt ctacaattca actaaggccg gcgtcgacgt cgtggatgaa
1441 ctgtgcgcga actataacgt gtcccggaac tctaagcggt ggcctatgac tctcttctac
1501 ggagtgctga atatggccgc aatcaacgcg tgcatcatct accgcaccaa caagaacgtg
1561 accatcaagc gcaccgagtt catcagatcg ctgggtttga gcatgatcta cgagcacctc
1621 cattcacgga acaagaagaa gaatatccct acttacctga ggcagcgtat cgagaagcag
1681 ttgggagaac caagcccgcg ccacgtgaac gtgccggggc gctacgtgcg gtgccaagat
1741 tgcccgtaca aaaaggaccg caaaaccaaa agatcgtgta acgcgtgcgc caaacctatc
1801 tgcatggagc atgccaaatt tctgtgtgaa aattgtgctg aactcgattc ctccctg.
```

In certain embodiments, the piggyBac or piggyBac-like transposase is hyperactive. A hyperactive piggyBac or piggyBac-like transposase is a transposase that is more active than the naturally occurring variant from which it is derived. In certain embodiments, the hyperactive piggyBac or piggyBac-like transposase enzyme is isolated or derived from Bombyx mori. In certain embodiments, the piggyBac or piggyBac-like transposase is a hyperactive variant of SEQ ID NO: 14505. In certain embodiments, the hyperactive piggyBac or piggyBac-like transposase comprises a sequence that is at least 90% identical to:

```
(SEQ ID NO: 14576)
    1 MDIERQEERI RAMLEEELSD YSDESSSEDE TDHCSEHEVN YDTEEERIDS VDVPSNSRQE
  61 EANAIIANES DSDPDDDLPL SLVRQRASAS RQMSGPHYTS KDGTKWYKNC QRPNVRLRSE
121 NIVTEQAQVK NIARDASTEY ECWNIFVTSD MLQEILTHTN SSIRWRQTKT AAENSSASTS
181 FYMQETTLCE LKALIGLLYI AGLIKSNRQS LKDLWRTDGT GVDIFRTTMS LQRFQFLQNN
241 IRFDDKSTRD ERKQTDNMAA FRSIFDQFVQ SCQNAYSPSE FLTIDEMLLS FRGRCLFRVY
301 IPNKPAKYGI KILALVDAKN FYVKNLEVYA GKQPSGPYAV SNRPFEVVER LIQPVARSHR
361 NVTFDNWFTG YELMLHLLNE YRLTSVGTVR KNKRQIPESF IRTDRQPNSS VFGFQKDITL
421 VSYAPKKNKV VVVMSTMHHD NSIDESTGEK QKPEMITFYN STKAGVDVVD ELCANYNVSR
481 NSKRWPMTLF YGVLNMAAIN ACIIYRTNKN VTIKRTEFIR SLGLSMIYEH LHSRNKKKNI
541 PTYLRQRIEK QLGEPSPRHV NVPGRYVRCQ DCPYKKDRKT KRSCNACAKP ICMEHAKFLC
601 ENCAELDSHL.
```

In certain embodiments, the hyperactive piggyBac or piggyBac-like transposase comprises SEQ ID NO: 14576. In certain embodiments, the hyperactive piggyBac or piggyBac-like transposase comprises a sequence of:

```
(SEQ ID NO: 14630)
    1 MDIERQEERI RAMLEEELSD YSDESSSEDE TDHCSEHEVN YDTEEERIDS VDVPSNSRQE
  61 EANAIIANES DSDPDDDLPL SLVRQRASAS RQVSGPFYTS KDGTKWYKNC QRPNVRLRSE
121 NIVTEQAQVK NIARDASTEY ECWNIFVTSD MLQEILTHTN SSIRWRQTKT AAENSSAETS
181 FYMQETTLCE LKALIGLLYI AGLIKSNRQS LKDLWRTDGT GVDIFRTTMS LQRFQFLLNN
241 IRFDDKSTRD ERKQTDNMAA FRSIFDQFVQ SCQNAYSPSE FLTIDEMLLS FRGRCLFRVY
301 IPNKPAKYGI KILALVDAKN FYVHNLEVYA GKQPSGPYAV SNRPFEVVER LIQPVARSHR
361 NVTFDNWFTG YEVMLHLLNE YRLTSVGTVR KNKRQIPESF IRTDRQPNSS VFGFQKDITL
421 VSYAPKKNKV VVVMSTMHHD NSIDESTGEK QKPEMITFYN STKAGVDVVD ELCANYNVSR
481 NSKRWPMTLF YGVLNMAAIN ACIIYRTNKN VTIKRTEFIR SLGLSMIYEH LHSRNKKKNI
541 PTYLRQRIEK QLGEPSPRHV NVPGRYVRCQ DCPYKKDRKT KRSCNACAKP ICMEHAKFLC
601 ENCAHLDS.
```

In certain embodiments, the hyperactive piggyBac or piggyBac-like transposase comprises a sequence of:

```
(SEQ ID NO: 14631)
    1 MDIERQEERI RAMLEEELSD YSDESSSEDE TDHCSEHEVN YDTEEERIDS VDVPSNSRQE
  61 EANAIIANES DSDPDDDLPL SLVRQRASAS RQVSGPFYTS KDGTKWYKNC QRPNVRLRSE
121 NIVTEQAQVK NIARDASTEY ECWNIFVTSD MLQEILTHTN SSIRWRQTKT AAENSSASTS
181 FYMQETTLCE LKALIGLLYI AGLIKSNRQS LKDLWRTDGT GVDIFRTTMS LQRFQFLLNN
241 IRFDDKSTRD ERKQTDNMAA FRSIFDQFVQ SCQNAYSPSE FLTIDEMLLS FRGRCLFRVY
301 IPNKPAKYGI KILALVDAKN FYVKNLEVYA GKQPSGPYAV SNRPFEVVER LIQPVARSHR
361 NVTFDNWFTG YELMLHLLNE YRLTSVGTVR KNKRQIPESF IRTDRQPNSS VFGFQKDITL
421 VSYAPKKNKV VVVMSTMHHD NSIDESTGEK QKPEMITFYN STKAGVDVVD ELCANYNVSR
481 NSKRWPMTLF YGVLNMAAIN ACIIYRTNKN VTIKRTEFIR SLGLSMIYEH LHSRNKKKNI
541 PTYLRQRIAM QLGEPSPRHV NVPGRYVRCQ DCPYKKDRKT KRSCNACAKP ICMEHAKFLC
601 ENCAELDSSL.
```

In certain embodiments, the hyperactive piggyBac or piggyBac-like transposase comprises a sequence of:

```
(SEQ ID NO: 14632)
    1 MDIERQEERI RAMLEEELSD YSDESSSEDE TDHCSEHEVN YDTEEERIDS VDVPSNSRQE
  61 EANAIIANES DSDPDDDLPL SLVRQRASAS RQVSGPFYTS KDGTKWYKNC QRPNVRLRSE
121 NIVTEQAQVK NIARDASTEY ECWNIFVTSD MLQEILTHTN SSIRWRQTKT AAENSSAETS
181 FYMQETTLCE LKALIGLLYI AGLIKSNRQS LKDLWRTDGT GVDIFRTTMS LQRFQFLLNN
241 IRFDDKSTRD ERKQTDNMAA FRSIFDQFVQ SCQNAYSPSE FLTIDEMLLS FRGRCLFRVY
301 IPNKPAKYGI KILALVDAKN FYVKNLEVYA GKQPSGPYAV SNRPFEVVER LIQPVARSHR
361 NVTFDNWFTG YELMLHLLNE YRLTSVGTVR KNKTQIPENF IRTDRQPNSS VFGFQKDITL
421 VSYAPKKNKV VVVMSTMHHD NSIDESTGEK QKPEMITFYN STKAGVDVVD ELQANYNVSR
481 NSKRWPMTLF YGVLNMAAIN ACIIYRTNKN VTIKRTEFIR SLGLSMIYEH LHSRNKKKNI
541 PTYLRQRIEK QLGEPSPRHV NVPGRYVRCQ DCPYKKDRKT KRSCNACAKP ICMEHAKFLC
601 ENCAELDSSL.
```

In certain embodiments, the hyperactive piggyBac or piggyBac-like transposase comprises a sequence of:

```
(SEQ ID NO: 14633)
    1 MDIERQEERI RAMLEEELSD YSDESSSEDE TDHCSEHEVN YDTEEERIDS VDVPSNSRQE
  61 EANAIIANES DSDPDDDLPL SLVRQRASAS RQVSGPFYTS KDGTKWYKNC QRPNVRLRSE
121 NIVTEQAQVK NIARDASTEY ECWNIFVTSD MLQEILTHTN SSIRWRQTKT AAENSSAETS
181 FYMQETTLCE LKALIGLLYI AGLIKSNRQS LKDLWRTDGT GVDIFRTTMS LQRFQFLQNN
241 IRFDDKSTRD ERKQTDNMAA FRSIFDQFVQ SCQNAYSPSE FLTIDEMLLS FRGRCLFRVY
301 IPNKPAKYGI KILALVDAKN FYVKNLEVYA GKQPSGPYAV SNRPFEVVER LIQPVARSHR
361 NVTFDNWFTG YELMLHLLNE YRLTSVGTVR KNKRQIPESF IRTDRQPNSS VFGFQKDITL
421 VSYAPKKNKV VVVMSTMHHD NSIDESTGEK QKPEMITFYN STKAGVDVVD ELCANYNVSR
481 NSKRWPMTLF YGVLNMAAIN ACIIYRTNKN VTIKRTEFIR SLGLSMIYEH LHSRNKKKNI
541 PTYLRQRIEK QLGEPSPRHV NVPGRYVRCQ DCPYKKDRKT KRSCNACAKP ICMEHAKFLC
601 ENCAELDSSL.
```

In certain embodiments, the hyperactive piggyBac or piggyBac-like transposase comprises a sequence of:

```
(SEQ ID NO: 14634)
    1 MDIERQEERI RAMLEEELSD YSDESSSEDE TDHCSEHEVN YDTEEERIDS VDVPSNSRQE
  61 EANAIIANES DSDPDDDLPL SLVRQRASAS RQVSGPFYTS KDGTKWYKNC QRPNVRLRSE
121 NIVTEQAQVK NIARDASTEY ECWNIFVTSD MLQEILTHTN SSIRHRQTKT AAENSSAETS
181 FYMQETTLCE LKALIALLYL AGLIKSNRQS LKDLWRTDGT GVDIFRTTMS LQRFQFLQNN
241 IRFDDKSTRD ERKQTDNMAA FRSIFDQFVQ CCQNAYSPSE FLTIDEMLLS FRGRCLFRVY
301 IPNKPAKYGI KILALVDAKN DYVVNLEVYA GKQPSGPYAV SNRPFEVVER LIQPVARSHR
361 NVTFDNWFTG YELMLHLLNE YRLTSVGTVR KNKRQIPESF IRTDRQPNSS VFGFQKDITL
421 VSYAPKKNKV VVVMSTMHHD NSIDESTGEK QKPEMITFYN STKAGVDVVD ELCANYNVSR
481 NSKRWPMTLF YGVLNMAAIN ACIIYRTNKN VTIKRTEFIR SLGLSMIYEH LHSRNKKKNI
541 PTYLRQRIEK QLGEPSSRHV NVKGRYVRCQ DCPYKKDRKT KRSCNACAKP ICMEHAKFLC
601 ENCAELDSSL.
```

In certain embodiments, the hyperactive piggyBac or piggyBac-like transposase is more active than the transposase of SEQ ID NO: 14505. In certain embodiments, the hyperactive piggyBac or piggyBac-like transposase is at least 90%, at least 95%, at least 96%, at least 97%, at least 98%, or at least 99% or any percentage in between identical to SEQ ID NO: 14505.

In certain embodiments, the hyperactive piggyBac or piggyBac-like transposase comprises an amino acid substitution at a position selected from 92, 93, 96, 97, 165, 178, 189, 196, 200, 201, 211, 215, 235, 238, 246, 253, 258, 261, 263, 271, 303, 321, 324, 330, 373, 389, 399, 402, 403, 404, 448, 473, 484, 507, 523, 527, 528, 543, 549, 550, 557, 601, 605, 607, 609, 610 or a combination thereof (relative to SEQ ID NO: 14505). In certain embodiments, the hyperactive piggyBac or piggyBac-like transposase comprises an amino acid substitution of Q92A, V93L, V93M, P96G, F97H, F97C, H165E, H165W, E178S, E178H, C189P, A196G, L200I, A201Q, L211A, W215Y, G2195, Q235Y, Q235G, Q238L, K246I, K253V, M258V, F261L, S263K, C271S, N303R, F321W, F321D, V324K, V324H, A330V, L373C, L373V, V389L, S399N, R402K, T403L, D404Q, D4045, D404M, N441R, G448W, E449A, V469T, C473Q, R484K T507C, G523A, I527M, Y528K Y543I, E549A, K550M, P5575, E601V, E605H, E605W, D607H, 5609H, L610I or any combination thereof. In certain embodiments, the hyperactive piggyBac or piggyBac-like transposase comprises an amino acid substitution of Q92A, V93L, V93M, P96G, F97H, F97C, H165E, H165W, E178S, E178H, C189P, A196G, L200I, A201Q, L211A, W215Y, G2195, Q235Y, Q235G, Q238L, K246I, K253V, M258V, F261L, S263K, C271S, N303R, F321W, F321D, V324K, V324H, A330V, L373C, L373V, V389L, S399N, R402K, T403L, D404Q, D4045, D404M, N441R, G448W, E449A, V469T, C473Q, R484K T507C, G523A, I527M, Y528K Y543I, E549A, K550M, P5575, E601V, E605H, E605W, D607H, 5609H and L610I.

In certain embodiments, the hyperactive piggyBac or piggyBac-like transposase comprises one or more substitutions of an amino acid that is not wild type, wherein the one or more substitutions a for wild type amino acid comprises a substitution of E4X, A12X, M13X, L14X, E15X, D20X, E24X, S25X, S26X, S27X, D32X, H33X, E36X, E44X, E45X, E46X, I48X, D49X, R58X, A62X, N63X, A64X, I65X, I66X, N68X, E69X, D71X, S72X, D76X, P79X, R84X, Q85X, A87X, S88X, Q92X, V93X, S94X, G95X, P96X, F97X, Y98X, T99X, I145X, S149X, D150X, L152X, E154X, T157X, N160X, S161X, S162X, H165X, R166X, T168X, K169X, T170X, A171X, E173X, S175X, S176X, E178X, T179X, M183X, Q184X, T186X, T187X, L188X, C189X, L194X, I195X, A196X, L198X, L200X, A201X, L203X, I204X, K205X, A206X, N207X, Q209X, S210X, L211X, K212X, D213X, L214X, W215X, R216X, T217X, G219X, V222X, D223X, I224X, T227X, M229X, Q235X, L237X, Q238X, N239X, N240X, P302X, N303X, P305X, A306X, K307X, Y308X, 1310X, K311X, I312X, L313X, A314X, L315X, V316X, D317X, A318X, K319X, N320X, F321X, Y322X, V323X, V324X, L326X, E327X, V328X, A330X, Q333X, P334X, S335X, G336X, P337X, A339X, V340X, S341X, N342X, R343X, P344X, F345X, E346X, V347X, E349X, I352X, Q353X, V355X, A356X, R357X, N361X, D365X, W367X, T369X, G370X, L373X, M374X, L375X, H376X, N379X, E380X, R382X, V386X, V389X, N392X, R394X, Q395X, S399X, F400X, I401X, R402XT403X, D404X, R405X, Q406X, P407X, N408X, S409X, S410X, V411X, F412X, F414X, Q415X, I418X, T419X, L420X, N428XV432X, M434X, D440X, N441X, S442X, I443X, D444X, E445X, G448X, E449X, Q451X, K452X, M455X, I456X, T457X, F458X, S461X, A464X, V466X, Q468X, V469X, E471X, L472X, C473X, A474X, K483X, W485X, T488X, L489X, Y491X, G492X, V493X, M496X, I499X, C502X, I503X, I507X, K509X, N510X, V511X, T512X, I513X, R515X, E517X, S521X, G523X, L524X, S525X, I527X, Y528X, E529X, H532X, S533X, N535X, K536X, K537X, N539X, I540X, T542X, Y543X, Q546X, E549X, K550X, Q551X, G553X, E554X, P555X, S556X, P557X, R558X, H559X, V560X, N561X, V562X, P563X, G564X, R565X, Y566X, V567X, Q570X, D571X, P573X, Y574X, K576X, K581X, S583X, A586X, A588X, E594X, F598X, L599X, E601X, N602X, C603X, A604X, E605X, L606X, D607X, S608X, S609X or L610X (relative to SEQ ID NO: 14505). A list of hyperactive amino acid substitutions can be found in U.S. Pat. No. 10,041,077, the contents of which are incorporated herein by reference in their entirety.

In certain embodiments, the piggyBac or piggyBac-like transposase is integration deficient. In certain embodiments, an integration deficient piggyBac or piggyBac-like transposase is a transposase that can excise its corresponding transposon, but that integrates the excised transposon at a lower frequency than a corresponding wild type transposase. In certain embodiments, the piggyBac or piggyBac-like transposase is an integration deficient variant of SEQ ID NO: 14505.

In certain embodiments, the excision competent, integration deficient piggyBac or piggyBac-like transposase comprises one or more substitutions of an amino acid that is not wild type, wherein the one or more substitutions a for wild type amino acid comprises a substitution of R9X, A12X, M13X, D20X, Y21K, D23X, E24X, S25X, S26X, S27X, E28X, E30X, D32X, H33X, E36X, H37X, A39X, Y41X, D42X, T43X, E44X, E45X, E46X, R47X, D49X, S50X, S55X, A62X, N63X, A64X, I66X, A67X, N68X, E69X, D70X, D71X, S72X, D73X, P74X, D75X, D76X, D77X, I78X, S81X, V83X, R84X, Q85X, A87X, S88X, A89X, S90X, R91X, Q92X, V93X, S94X, G95X, P96X, F97X, Y98X, T99X, W012X, G103X, Y107X, K108X, L117X, I122X, Q128X, I312X, D135X, S137X, E139X, Y140X, I145X, S149X, D150X, Q153X, E154X, T157X, S161X, S162X, R164X, H165X, R166X, Q167X, T168X, K169X, T170X, A171X, A172X, E173X, R174X, S175X, S176X, A177X, E178X, T179X, S180X, Y182X, Q184X, E185X, T187X, L188X, C189X, L194X, I195X, A196X, L198X, L200X, A201X, L203X, I204X, K205X, N207X, Q209X, L211X, D213X, L214X, W215X, R216X, T217X, G219X, T220X, V222X, D223X, I224X, T227X, T228X, F234X, Q235X, L237X, Q238X, N239X, N240X, N303X, K304X, I310X, I312X, L313X, A314X, L315X, V316X, D317X, A318X, K319X, N320X, F321X, Y322X, V323X, V324X, N325X, L326X, E327X, V328X, A330X, G331X, K332X, Q333X, S335X, P337X, P344X, F345X, E349X, H359X, N361X, V362X, D365X, F368X, Y371X, E372X, L373X, H376X, E380X, R382X, R382X, V386X, G387X, T388X, V389X, K391X, N392X, R394X, Q395X, E398X, S399X, F400X, I401X, R402XT403X, D404X, R405X, Q406X, P407X, N408X, 5409X, S410X, Q415X, K416X, A424X, K426X, N428X, V430X, V432X, V433X, M434X, D436X, D440X, N441X, S442X, I443X, D444X, E445X, S446X, T447X, G448X, E449X, K450X, Q451X, E454X, M455X, I456X, T457X, F458X, S461X, A464X, V466X, Q468X, V469X, C473X, A474X, N475X, N477X, K483X, R484X, P486X, T488X, L489X, G492X, V493X, M496X, I499X, I503X, Y505X, T507X, N510X, V511X, T512X, I513X, K514X, T516X, E517X, S521X, G523X, L524X, S525X, I527X, Y528X, L531X, H532X, S533X, N535X, 1540X, T542X, Y543X, R545X, Q546X, E549X, L552X, G553X, E554X, P555X, S556X, P557X, R558X, H559X, V560X, N561X, V562X, P563X, G564X, V567X, Q570X, D571X, P573X, Y574X, K575X, K576X, N585X, A586X, M593X, K596X, E601X, N602X, A604X, E605X, L606X, D607X, S608X, S609X or L610X (relative to SEQ ID NO: 14505). A list of integration deficient amino acid substitutions can be found in U.S. Pat. No. 10,041,077, the contents of which are incorporated by reference in their entirety.

In certain embodiments, the integration deficient piggyBac or piggyBac-like transposase comprises a sequence of:

```
(SEQ ID NO: 14606)
    1 MDIERQEERI RAMLEEELSD YSDESSSEDE TDHCSEHEVN YDTEEERIDS VDVPSNSRQE
  61 EANAIIANES DSDPDDDLPL SLVRQRASAS RQVSGPFYTS KDGTKWYKNC QRPNVRLRSE
121 NIVTEQAQVK NIARDASTEY ECWNIFVTSD MLQEILTHTN SSIRHRQTKT AAENSSAETS
181 FYMQETTLCE LKALIALLYL AGLIKSNRQS LKDLWRKDGT GVDIFRTTMS LQRFQFLLNN
241 IRFDDISTRD ERKQTDNMAA FRSIFDQFVQ CCQNAYSPSE FLTIDEMLLS FRGRCLFRVY
301 IPNKPAKYGI KILALVDAKN FYVVNLEVYA GKQPSGPYAV SNRPFEVVER LIQPVARSHR
361 NVTFDNWFTG YELMLHLLNE YRLTSVGTVR KNKRQIPESF IRTDRQPNSS VFGFQKDITL
421 VSYAPKKNKV VVVMSTMHHD NSIDESTGEK QKPEMITFYN STKAGVDVVD ELCANYNVSR
481 NSKKWPMTLF YGVLNMAAIN ACIIYRTNKN VTIKRTEFIR SLGLSMMYEH LHSRNKKKNI
541 PTYLRQRIEK QLGEPVPRHV NVPGRYVRCQ DCPYKKDRKT KRSCNACAKP ICMEHAKFLC
601 ENCAELDSSL.
```

In certain embodiments, the integration deficient piggyBac or piggyBac-like transposase comprises a sequence of:

```
(SEQ ID NO: 14607)
    1 MDIERQEERI RAMLEEELSD YSDESSSEDE TDHCSEHEVN YDTEEERIDS VDVPSNSRQE
  61 EANAIIANES DSDPDDDLPL SLVRQRASAS RQVSGPFYTS KDGTKWYKNC QRPNVRLRSE
121 NIVTEQAQVK NIARDASTEY ECWNIFVTSD MLQEILTHTN SSIRHRQTKT AAENSSAETS
181 FYMQETTLCE LKALIGLLYL AGLIKSNRQS LKDLWRTDGT GVDIFRTTMS LQRFYFLQNN
241 IRFDDKSTLD ERKQTDNMAA FRSIFDQFVQ SCQNAYSPSE FLTIDEMLLS FRGRCLFRVY
301 IPNKPAKYGI KILALVDAKN FYVVNLEVYA GKQPSGPYAV SNRPFEVVER LIQPVARSHR
361 NVTFDNWFTG YELMLHLLNE YRLTSVGTVR KNKRQIPESF IRTDRQPNSS VFGFQKDITL
421 VSYAPKKNKV VVVMSTMHHD NSIDESTGEK QKPEMITFYN STKAGVDVVD ELCANYNVSR
481 NSKRWPMTLF YGVLNMAAIN ACIIYRTNKN VTIKRTEFIR SLGLSMIYEH LHSRNKKKNI
541 PTYLRQRIEK QLGEPSPRHV NYPGRYVRCQ DCPYKKDRKT KRSCNACAKP ICMEHAKFLC
601 VNCAELDSSL.
```

In certain embodiments, the piggyBac or piggyBac-like transposase that is integration deficient comprises a sequence of:

```
(SEQ ID NO: 14608)
    1 MDIERQEERI RAMLEEELSD YSDESSSEDE TDHCSEHEVN YDTEEERIDS VDVPSNSRQE
  61 EANAIIANES DSDPDDDLPL SLVRQRASAS RQVSGPFYTS KDGTKWYKNC QRPNVRLRSE
121 NIVTEQAQVK NIARDASTEY ECWNIFVTSD MLQEILTHTN SSIRHRQTKT AAENSSAETS
181 FYMQETTLCE LKALIALLYL AGLIKSNRQS LKDLWRKDGT GVDIFRTTMS LQRFQFLLNN
241 IRFDDKSTRD ERKQTDNMAA FRSIFDQFVQ CCQNAYSPSE FLTIDEMLLS FRGRCLFRVY
301 IPNKPAKYGI KILALVDAKN DYVVNLEVYA GKQPSGPYAV SNRPFEVVER LIQPVARSHR
361 NVTFDNWFTG YECMLHLLNE YRLTSVGTVR KNKRQIPESF IRTDRQPNSS VFGFQKDITL
421 VSYAPKKNKV VVVMSTMHHD NSIDESTGEK QKPEMITFYN STKAGVDVVD ELCANYNVSR
481 NSKKWPMTLF YGVLNMAAIN ACIIYRTNKN VTIKRTEFIR SLGLSMIKEH LHSRNKKKNI
541 PTYLRQRIEK QLGEPSPRHV NVPGRYVRCQ DCPYKKDRKT KRSCNACAKP ICMEHAKFLC
601 ENCAELDSSL.
```

In certain embodiments, the integration deficient transposase comprises a sequence that is at least 90% identical to SEQ ID NO: 14608.

In certain embodiments, the piggyBac or piggyBac-like transposon is isolated or derived from Bombyx mori. In certain embodiments, the piggyBac or piggyBac-like transposon comprises a sequence of:

```
(SEQ ID NO: 14506)
    1 ttatcccggc gagcatgagg cagggtatct cataccctgg taaaatttta aagttgtgta
  61 ttttataaaa ttttcgtctg acaacactag cgcgctcagt agctggaggc aggagcgtgc
121 gggaggggat agtggcgtga tcgcagtgtg gcacgggaca ccggcgagat attcgtgtgc
181 aaacctgttt cgggtatgtt ataccctgcc tcattgttga cgtatttttt ttatgtaatt
241 tttccgatta ttaatttcaa ctgttttatt ggtattttta tgttatccat tgttcttttt
301 ttatgattta ctgtatcggt tgtctttcgt tcctttagtt gagttttttt ttattatttt
361 cagtttttga tcaaa.
```

In certain embodiments, the piggyBac or piggyBac-like transposon comprises a sequence of:

```
(SEQ ID NO: 14507)
    1 tcatattttt agtttaaaaa aataattata tgttttataa tgaaaagaat ctcattatct
  61 ttcagtatta ggttgattta tattccaaag aataatattt ttgttaaatt gttgattttt
121 gtaaacctct aaatgtttgt tgctaaaatt actgtgttta agaaaaagat taataaataa
181 taataatttc ataattaaaa acttctttca ttgaatgcca ttaaataaac cattatttta
241 caaaataaga tcaacataat tgagtaaata ataataagaa caatattata gtacaacaaa
301 atatgggtat gtcataccct gccacattct tgatgtaact ttttttcacc tcatgctcgc
361 cgggttat.
```

In certain embodiments, the piggyBac or piggyBac-like transposon comprises a sequence of:

```
(SEQ ID NO: 14508)
    1 ttatcccggc gagcatgagg cagggtatct cataccctgg taaaatttta aagttgtgta
  61 ttttataaaa ttttcgtctg acaacactag cgcgctcagt agctggaggc aggagcgtgc
121 gggaggggat agtggcgtga tcgcagtgtg gcacgggaca ccggcgagat attcgtgtgc
181 aaacctgttt cgggtatgtt ataccctgcc tcat.
```

In certain embodiments, the piggyBac™ (PB) or piggyBac-like transposon comprises a sequence of:

```
(SEQ ID NO: 14509)
    1 taaataataa taatttcata attaaaaact tctttcattg aatgccatta aataaaccat
  61 tattttacaa aataagatca acataattga gtaaataata ataagaacaa tattatagta
121 caacaaaata tgggtatgtc ataccctgcc acattcttga tgtaactttt tttcacctca
181 tgctcgccgg gttat.
```

In certain embodiments, the piggyBac or piggyBac-like transposon comprises a 5′ sequence corresponding to SEQ ID NO: 14506 and a 3′ sequence corresponding to SEQ ID NO: 14507. In certain embodiments, one piggyBac or piggyBac-like transposon end is at least 85%, at least 90%, at least 95%, at least 98%, at least 99% identical or any percentage in between identical to SEQ ID NO: 14506 and the other piggyBac or piggyBac-like transposon end is at least 85%, at least 90%, at least 95%, at least 98%, at least 99% or any percentage in between identical to SEQ ID NO: 14507. In certain embodiments, the piggyBac or piggyBac-like transposon comprises SEQ ID NO: 14506 and SEQ ID NO: 14507 or SEQ ID NO: 14509. In certain embodiments, the piggyBac or piggyBac-like transposon comprises SEQ ID NO: 14508 and SEQ ID NO: 14507 or SEQ ID NO: 14509. In certain embodiments, the 5′ and 3′ transposon ends share a 16 bp repeat sequence at their ends of CCCGGCGAGCATGAGG (SEQ ID NO: 14510) immediately adjacent to the 5′-TTAT-3 target insertion site, which is inverted in the orientation in the two ends. In certain embodiments, 5′ transposon end begins with a sequence comprising 5′-TTATCCCGGCGAGCATGAGG-3 (SEQ ID NO: 14511), and the 3′ transposon ends with a sequence comprising the reverse complement of this sequence: 5′-CCTCATGCTCGCCGGGTTAT-3′ (SEQ ID NO: 14512).

In certain embodiments, the piggyBac or piggyBac-like transposon comprises one end comprising at least 14, 16, 18, 20, 30 or 40 contiguous nucleotides of SEQ ID NO: 14506 or SEQ ID NO: 14508. In certain embodiments, the piggyBac or piggyBac-like transposon comprises one end comprising at least 14, 16, 18, 20, 30 or 40 contiguous nucleotides of SEQ ID NO: 14507 or SEQ ID NO: 14509. In certain embodiments, the piggyBac or piggyBac-like transposon comprises one end with at least 90% identity to SEQ ID NO: 14506 or SEQ ID NO: 14508. In certain embodiments, the piggyBac or piggyBac-like transposon comprises one end with at least 90% identity to SEQ ID NO: 14507 or SEQ ID NO: 14509.

In certain embodiments, the piggyBac or piggyBac-like transposon comprises a sequence of:

```
(SEQ ID NO: 14515)
    1 ttaacccggc gagcatgagg cagggtatct cataccctgg taaaatttta aagttgtgta
  61 ttttataaaa ttttcgtctg acaacactag cgcgctcagt agctggaggc aggagcgtgc
121 gggaggggat agtggcgtga tcgcagtgtg gcacgggaca ccggcgagat attcgtgtgc
181 aaacctgttt cgggtatgtt ataccctgcc tcattgttga cgtatttttt ttatgtaatt
241 tttccgatta ttaatttcaa ctgttttatt ggtattttta tgttatccat tgttcttttt
301 ttatgattta ctgtatcggt tgtctttcgt tcctttagtt gagttttttt ttattatttt
361 cagtttttga tcaaa.
```

In certain embodiments, the piggyBac or piggyBac-like transposon comprises a sequence of:

```
(SEQ ID NO: 14516)
    1 tcatattttt agtttaaaaa aataattata tgttttataa tgaaaagaat ctcattatct
  61 ttcagtatta ggttgattta tattccaaag aataatattt ttgttaaatt gttgattttt
121 gtaaacctct aaatgtttgt tgctaaaatt actgtgttta agaaaaagat taataaataa
181 taataatttc ataattaaaa acttctttca ttgaatgcca ttaaataatt cattatttta
241 caaaataaga tcaacataat tgagtaaata ataataagaa caatattata gtacaacaaa
301 atatgggtat gtcataccct tttttttttt tttttttttt ttttttcggg tagagggccg
361 aacctcctac gaggtccccg cgcaaaaggg gcgcgcgggg tatgtgagac tcaacgatct
421 gcatggtgtt gtgagcagac cgcgggccca aggattttag agcccaccca ctaaacgact
481 cctctgcact cttacacccg acgtccgatc ccctccgagg tcagaacccg gatgaggtag
541 gggggctacc gcggtcaaca ctacaaccag acggcgcggc tcaccccaag gacgcccagc
601 cgacggagcc ttcgaggcga atcgaaggct ctgaaacgtc ggccgtctcg gtacggcagc
661 ccgtcgggcc gcccagacgg tgccgctggt gtcccggaat accccgctgg accagaacca
721 gcctgccggg tcgggacgcg atacaccgtc gaccggtcgc tctaatcact ccacggcagc
781 gcgctagagt gctggta.
```

In certain embodiments, the piggyBac or piggyBac-like transposon comprises a sequence of CCCGGCGAGCATGAGG (SEQ ID NO: 14510). In certain embodiments, the piggyBac or piggyBac-like transposon comprises an ITR sequence of SEQ ID NO: 14510. In certain embodiments, the piggyBac or piggyBac-like transposon comprises a sequence of TTATCCCGGCGAGCATGAGG (SEQ ID NO: 14511). In certain embodiments, the piggyBac or piggyBac-like transposon comprises at least 16 contiguous nucleotides from SEQ ID NO: 14511. In certain embodiments, the piggyBac or piggyBac-like transposon comprises a sequence of CCTCATGCTCGCCGGGTTAT (SEQ ID NO: 14512). In certain embodiments, the piggyBac or piggyBac-like transposon comprises at least 16 contiguous nucleotides from SEQ ID NO: 14512. In certain embodiments, the piggyBac or piggyBac-like transposon comprises one end comprising at least 16 contiguous nucleotides from SEQ ID NO: 14511 and one end comprising at least 16 contiguous nucleotides from SEQ ID NO: 14512. In certain embodiments, the piggyBac or piggyBac-like transposon comprises SEQ ID NO: 14511 and SEQ ID NO: 14512. In certain embodiments, the piggyBac or piggyBac-like transposon comprises a sequence of TTAACCCGGCGAGCATGAGG (SEQ ID NO: 14513). In certain embodiments, the piggyBac or piggyBac-like transposon comprises a sequence of CCTCATGCTCGCCGGGTTAA (SEQ ID NO: 14514).

In certain embodiments, the piggyBac or piggyBac-like transposon may have ends comprising SEQ ID NO: 14506 and SEQ ID NO: 14507, or a variant of either or both of these having at least 90% sequence identity to SEQ ID NO: 14506 or SEQ ID NO: 14507, and the piggyBac or piggyBac-like transposase has the sequence of SEQ ID NO: 14504 or SEQ ID NO: 14505, or a sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identity to SEQ ID NO: 14504 or SEQ ID NO: 14505. In certain embodiments, the piggyBac or piggyBac-like transposon comprises a heterologous polynucleotide inserted between a pair of inverted repeats, where the transposon is capable of transposition by a piggyBac or piggyBac-like transposase having at least 5%, 10%, 15%, 20%, 25%, 30%, 35%, 40%, 45%, 50%, 55%, 60%, 65%, 70%, 75%, 80%, 85%, 90%, 95%, 99% or any percentage in between identity to SEQ ID NO: 14504 or SEQ ID NO: 14505. In certain embodiments, the transposon comprises two transposon ends, each of which comprises SEQ ID NO: 14510 in inverted orientations in the two transposon ends. In certain embodiments, each inverted terminal repeat (ITR) is at least 90% identical to SEQ ID NO: 14510.

In certain embodiments, the piggyBac or piggyBac-like transposon is capable of insertion by a piggyBac or piggyBac-like transposase at the sequence 5′-TTAT-3 within a target nucleic acid. In certain embodiments, one end of the piggyBac or piggyBac-like transposon comprises at least 16 contiguous nucleotides from SEQ ID NO: 14506 and the other transposon end comprises at least 16 contiguous nucleotides from SEQ ID NO: 14507. In certain embodiments, one end of the piggyBac or piggyBac-like transposon comprises at least 17, at least 18, at least 19, at least 20, at least 22, at least 25, at least 30 contiguous nucleotides from SEQ ID NO: 14506 and the other transposon end comprises at least 17, at least 18, at least 19, at least 20, at least 22, at least 25, at least 30 contiguous nucleotides from SEQ ID NO: 14507.

In certain embodiments, the piggyBac or piggyBac-like transposon comprises transposon ends (each end comprising an ITR) corresponding to SEQ ID NO: 14506 and SEQ ID NO: 14507, and has a target sequence corresponding to 5′-TTAT3′. In certain embodiments, the piggyBac or piggyBac-like transposon also comprises a sequence encoding a transposase (e.g. SEQ ID NO: 14505). In certain embodiments, the piggyBac or piggyBac-like transposon comprises one transposon end corresponding to SEQ ID NO: 14506 and a second transposon end corresponding to SEQ ID NO: 14516. SEQ ID NO: 14516 is very similar to SEQ ID NO: 14507, but has a large insertion shortly before the ITR. Although the ITR sequences for the two transposon ends are identical (they are both identical to SEQ ID NO: 14510), they have different target sequences: the second transposon has a target sequence corresponding to 5′-TTAA-3′, providing evidence that no change in ITR sequence is necessary to modify the target sequence specificity. The piggyBac or piggyBac-like transposase (SEQ ID NO: 14504), which is associated with the 5′-TTAA-3′ target site differs from the 5′-TTAT-3′-associated transposase (SEQ ID NO: 14505) by only 4 amino acid changes (D322Y, S473C, A507T, H582R). In certain embodiments, the piggyBac or piggyBac-like transposase (SEQ ID NO: 14504), which is associated with the 5′-TTAA-3′ target site is less active than the 5′-TTAT-3′-associated piggyBac or piggyBac-like transposase (SEQ ID NO: 14505) on the transposon with 5′-TTAT-3′ ends. In certain embodiments, piggyBac or piggyBac-like transposons with 5′-TTAA-3′ target sites can be converted to piggyBac or piggyBac-like transposases with 5′-TTAT-3 target sites by replacing 5′-TTAA-3′ target sites with 5′-TTAT-3′. Such transposons can be used either with a piggyBac or piggyBac-like transposase such as SEQ ID NO: 14504 which recognizes the 5′-TTAT-3′ target sequence, or with a variant of a transposase originally associated with the 5′-TTAA-3′ transposon. In certain embodiments, the high similarity between the 5′-TTAA-3′ and 5′-TTAT-3′ piggyBac or piggyBac-like transposases demonstrates that very few changes to the amino acid sequence of a piggyBac or piggyBac-like transposase alter target sequence specificity. In certain embodiments, modification of any piggyBac or piggyBac-like transposon-transposase gene transfer system, in which 5′-TTAA-3′ target sequences are replaced with 5′-TTAT-3′-target sequences, the ITRs remain the same, and the transposase is the original piggyBac or piggyBac-like transposase or a variant thereof resulting from using a low-level mutagenesis to introduce mutations into the transposase. In certain embodiments, piggyBac or piggyBac-like transposon transposase transfer systems can be formed by the modification of a 5′-TTAT-3′-active piggyBac or piggyBac-like transposon-transposase gene transfer systems in which 5′-TTAT-3′ target sequences are replaced with 5′-TTAA-3′-target sequences, the ITRs remain the same, and the piggyBac or piggyBac-like transposase is the original transposase or a variant thereof.

In certain embodiments, the piggyBac or piggyBac-like transposon is isolated or derived from Bombyx mori. In certain embodiments, the piggyBac or piggyBac-like transposon comprises a sequence of:

```
(SEQ ID NO: 14577)
    1 cccggcgagc atgaggcagg gtatctcata ccctggtaaa attttaaagt tgtgtatttt
  61 ataaaatttt cgtctgacaa cactagcgcg ctcagtagct ggaggcagga gcgtgcggga
121 ggggatagtg gcgtgatcgc agtgtggcac gggacaccgg cgagatattc gtgtgcaaac
181 ctgtttcggg tatgttatac cctgcctcat tgttgacgta t.
```

In certain embodiments, the piggyBac or piggyBac-like transposon comprises a sequence of:

```
(SEQ ID NO: 14578)
    1 tttaagaaaa agattaataa ataataataa tttcataatt aaaaacttct ttcattgaat
  61 gccattaaat aaaccattat tttacaaaat aagatcaaca taattgagta aataataata
121 agaacaatat tatagtacaa caaaatatgg gtatgtcata ccctgccaca ttcttgatgt
181 aacttttttt cacctcatgc tcgccggg.
```

In certain embodiments, the transposon comprises at least 16 contiguous bases from SEQ ID NO: 14577 and at least 16 contiguous bases from SEQ ID NO: 14578, and inverted terminal repeats that are at least 87% identical to CCCGGCGAGCATGAGG (SEQ ID NO: 14510). In certain embodiments, the piggyBac or piggyBac-like transposon comprises a sequence of:

```
(SEQ ID NO: 14595)
    1 cccggcgagc atgaggcagg gtatctcata ccctggtaaa attttaaagt tgtgtatttt
  61 ataaaatttt cgtctgacaa cactagcgcg ctcagtagct ggaggcagga gcgtgcggga
121 ggggatagtg gcgtgatcgc agtgtggcac gggacaccgg cgagatattc gtgtgcaaac
181 ctgtttcggg tatgttatac cctgcctcat tgttgacgta ttttttttat gtaatttttc
241 cgattattaa tttcaactgt tttattggta tttttatgtt atccattgtt ctttttttat
301 gatttactgt atcggttgtc tttcgttcct ttagttgagt ttttttttat tattttcagt
361 ttttgatcaa a.
```

In certain embodiments, the piggyBac or piggyBac-like transposon comprises a sequence of:

```
(SEQ ID NO: 14596)
    1 tcatattttt agtttaaaaa aataattata tgttttataa tgaaaagaat ctcattatct
  61 ttcagtatta ggttgattta tattccaaag aataatattt ttgttaaatt gttgattttt
121 gtaaacctct aaatgtttgt tgctaaaatt actgtgttta agaaaaagat taataaataa
181 taataatttc ataattaaaa acttctttca ttgaatgcca ttaaataaac cattatttta
241 caaaataaga tcaacataat tgagtaaata ataataagaa caatattata gtacaacaaa
301 atatgggtat gtcataccct gccacattct tgatgtaact ttttttcacc tcatgctcgc
361 cggg.
```

In certain embodiments, the piggyBac or piggyBac-like transposon comprises SEQ ID NO: 14595 and SEQ ID NO: 14596, and is transposed by the piggyBac or piggyBac-like transposase of SEQ ID NO: 14505. In certain embodiments, the ITRs of SEQ ID NO: 14595 and SEQ ID: 14596 are not flanked by a 5′-TTAA-3′ sequence. In certain embodiments, the ITRs of SEQ ID NO: 14595 and SEQ ID: 14596 are flanked by a 5′-TTAT-3′ sequence.

In certain embodiments, the piggyBac or piggyBac-like transposon comprises a sequence of:

```
(SEQ ID NO: 14597)
    1 cccggcgagc atgaggcagg gtatctcata ccctggtaaa attttaaagt tgtgtatttt
  61 ataaaatttt cgtctgacaa cactagcgcg ctcagtagct ggaggcagga gcgtgcggga
121 ggggatagtg gcgtgatcgc agtgtggcac gggacaccgg cgagatattc gtgtgcaaac
181 ctgtttcggg tatgttatac cctgcctcat tgttgacgta ttttttttat gtaatttttc
241 cgattattaa tttcaactgt tttattggta tttttatgtt atccattgtt ctttttttat
301 g.
```

In certain embodiments, the piggyBac or piggyBac-like transposon comprises a sequence of:

```
(SEQ ID NO: 14598)
    1 cagggtatct cataccctgg taaaatttta aagttgtgta ttttataaaa ttttcgtctg
  61 acaacactag cgcgctcagt agctggaggc aggagcgtgc gggaggggat agtggcgtga
121 tcgcagtgtg gcacgggaca ccggcgagat attcgtgtgc aaacctgttt cgggtatgtt
181 ataccctgcc tcattgttga cgtatttttt ttatgtaatt tttccgatta ttaatttcaa
241 ctgttttatt ggtattttta tgttatccat tgttcttttt ttatg.
```

In certain embodiments, the piggyBac or piggyBac-like transposon comprises a sequence of:

```
(SEQ ID NO: 14599)
    1 cagggtatct cataccctgg taaaatttta aagttgtgta ttttataaaa ttttcgtctg
  61 acaacactag cgcgctcagt agctggaggc aggagcgtgc gggaggggat agtggcgtga
121 tcgcagtgtg gcacgggaca ccggcgagat attcgtgtgc aaacctgttt cgggtatgtt
181 ataccctgcc tcattgttga cgtat.
```

In certain embodiments, the 5′ end of the piggyBac or piggyBac-like transposon comprises a sequence of SEQ ID NO: 14577, SEQ ID NO: 14595, or SEQ ID NOs: 14597-14599. In certain embodiments, the 5′ end of the piggyBac or piggyBac-like transposon is preceded by a 5′ target sequence.
In certain embodiments, the piggyBac or piggyBac-like transposon comprises a sequence of:

```
(SEQ ID NO: 14600)
    1 tcatattttt agtttaaaaa aataattata tgttttataa tgaaaagaat ctcattatct
  61 ttcagtatta ggttgattta tattccaaag aataatattt ttgttaaatt gttgattttt
121 gtaaacctct aaatgtttgt tgctaaaatt actgtgttta agaaaaagat taataaataa
181 taataatttc ataattaaaa acttctttca ttgaatgcca ttaaataaac cattatttta
241 caaaataaga tcaacataat tgagtaaata ataataagaa caatattata gtacaacaaa
301 atatgggtat gtcataccct gccacattct tgatgtaact ttttttcacc tcatgctcgc
361 cggg.
```

In certain embodiments, the piggyBac or piggyBac-like transposon comprises a sequence of:

```
(SEQ ID NO: 14601)
    1 tttaagaaaa agattaataa ataataataa tttcataatt aaaaacttct ttcattgaat
  61 gccattaaat aaaccattat tttacaaaat aagatcaaca taattgagta aataataata
121 agaacaatat tatagtacaa caaaatatgg gtatgtcata ccctgccaca ttcttgatgt
181 aacttttttt ca.
```

In certain embodiments, the piggyBac or piggyBac-like transposon comprises a sequence of:

```
(SEQ ID NO: 14602)
    1 cccggcgagc atgaggcagg gtatctcata ccctggtaaa attttaaagt tgtgtatttt
  61 ataaaatttt cgtctgacaa cactagcgcg ctcagtagct ggaggcagga gcgtgcggga
121 ggggatagtg gcgtgatcgc agtgtggcac gggacaccgg cgagatattc gtgtgcaaac
181 ctgtttcggg tatgttatac cctgcctcat tgttgacgta ttttttttat gtaatttttc
241 cgattattaa tttcaactgt tttattggta tttttatgtt atccattgtt ctttttttat
301 gatttactgt atcggttgtc tttcgttcct ttagttgagt ttttttttat tattttcagt
361 ttttgatcaa a.
```

In certain embodiments, the 3′ end of the piggyBac or piggyBac-like transposon comprises a sequence of SEQ ID NO: 14578, SEQ ID NO: 14596, or SEQ ID NOs: 14600-14601. In certain embodiments, the 3′ end of the piggyBac or piggyBac-like transposon is followed by a 3′ target sequence. In certain embodiments, the transposon is transposed by the transposase of SEQ ID NO: 14505. In certain embodiments, the 5′ and 3′ ends of the piggyBac or piggyBac-like transposon share a 16 bp repeat sequence of SEQ ID NO: 14510 in inverted orientation and immediately adjacent to the target sequence. In certain embodiments, the 5′ transposon end begins with SEQ ID NO: 14510, and the 3′ transposon end ends with the reverse complement of SEQ ID NO: 14510, 5′-CCTCATGCTCGCCGGG-3′ (SEQ ID NO: 14603). In certain embodiments, the piggyBac or piggyBac-like transposon comprises an ITR with at least 93%, at least 87%, or at least 81% or any percentage in between identity to SEQ ID NO: 14510 or SEQ ID NO: 14603. In certain embodiments, the piggyBac or piggyBac-like transposon comprises a target sequence followed by a 5′ transposon end comprising a sequence selected from SEQ ID NOs: 14577, 14595 or 14597 and a 3′ transposon end comprising SEQ ID NO: 14578 or 14596 followed by a target sequence. In certain embodiments, the piggyBac or piggyBac like transposon comprises one end that comprises a sequence that is at least 90%, at least 95% or at least 99% or any percentage in between identical to SEQ ID NO: 14577 and one end that comprises a sequence that is at least 90%, at least 95% or at least 99% or any percentage in between identical to SEQ ID NO: 14578. In certain embodiments, one transposon end comprises at least 14, at least 16, at least 18 or at least 20 contiguous bases from SEQ ID NO: 14577 and one transposon end comprises at least 14, at least 16, at least 18 or at least 20 contiguous bases from SEQ ID NO: 14578.

In certain embodiments, the piggyBac or piggyBac-like transposon comprises two transposon ends wherein each transposon ends comprises a sequence that is at least 81% identical, at least 87% identical or at least 93% identical or any percentage in between identical to SEQ ID NO: 14510 in inverted orientation in the two transposon ends. One end may further comprise at least 14, at least 16, at least 18 or at least 20 contiguous bases from SEQ ID NO: 14599, and the other end may further comprise at least 14, at least 16, at least 18 or at least 20 contiguous bases from SEQ ID NO: 14601. The piggyBac or piggyBac-like transposon may be transposed by the transposase of SEQ ID NO: 14505, and the transposase may optionally be fused to a nuclear localization signal.

In certain embodiments, the piggyBac or piggyBac-like transposon comprises SEQ ID NO: 14595 and SEQ ID NO: 14596 and the piggyBac or piggyBac-like transposase comprises SEQ ID NO: 14504 or SEQ ID NO: 14505. In certain embodiments, the piggyBac or piggyBac-like transposon comprises SEQ ID NO: 14597 and SEQ ID NO: 14596 and the piggyBac or piggyBac-like transposase comprises SEQ ID NO: 14504 or SEQ ID NO: 14505. In certain embodiments, the piggyBac or piggyBac-like transposon comprises SEQ ID NO: 14595 and SEQ ID NO: 14578 and the piggyBac or piggyBac-like transposase comprises SEQ ID NO: 14504 or SEQ ID NO: 14505. In certain embodiments, the piggyBac or piggyBac-like transposon comprises SEQ ID NO: 14602 and SEQ ID NO: 14600 and the piggyBac or piggyBac-like transposase comprises SEQ ID NO: 14504 or SEQ ID NO: 14505.

In certain embodiments, the piggyBac or piggyBac-like transposon comprises a 5′ end comprising 1, 2, 3, 4, 5, 6, or 7 sequences selected from ATGAGGCAGGGTAT (SEQ ID NO: 14614), ATACCCTGCCTCAT (SEQ ID NO: 14615), GGCAGGGTAT (SEQ ID NO: 14616), ATACCCTGCC (SEQ ID NO: 14617), TAAAATTTTA (SEQ ID NO: 14618), ATTTTATAAAAT (SEQ ID NO: 14619), TCATACCCTG (SEQ ID NO: 14620) and TAAATAATAATAA (SEQ ID NO: 14621). In certain embodiments, the piggyBac or piggyBac-like transposon comprises a 3′ end comprising 1, 2 or 3 sequences selected from SEQ ID NO: 14617, SEQ ID NO: 14620 and SEQ ID NO: 14621.

In certain embodiments of the methods of the disclosure, the transposase enzyme is a piggyBac or piggyBac-like transposase enzyme. In certain embodiments, the piggyBac or piggyBac-like transposase enzyme is isolated or derived from Xenopus tropicalis. The piggyBac or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14517)
    1 MAKRFYSAEE AAAHCMASSS EEFSGSDSEY VPPASESDSS TEESWCSSST VSALEEPMEV
  61 DEDVDDLEDQ EAGDRADAAA GGEPAWGPPC NFPPEIPPFT TVPGVKVDTS NFEPINFFQL
121 FMTEAILQDM VLYTNVYAEQ YLTQNPLPRY ARAHAWHPTD IAEMKRFVGL TLAMGLIKAN
181 SLESYWDTTT VLSIPVFSAT MSRNRYQLLL RFLHFNNNAT AVPPDQPGHD RLHKLRPLID
241 SLSERFAAVY TPCQNICIDE SLLLFKGRLQ FRQYIPSKRA RYGIKFYKLC ESSSGYTSYF
301 LIYEGKDSKL DPPGCPPDLT VSGKIVWELI SPLLGQGFHL YVDNFYSSIP LFTALYCLDT
361 PACGTINRNR KGLPRALLDK KLNRGETYAL RKNELLAIKF FDKKNVFMLT SIHDESVIRE
421 QRVGRPPKNK PLCSKEYSKY MGGVDRTDQL QHYYNATRKT RAWYKKVGIY LIQMALRNSY
481 IVYKAAVPGP KLSYYKYQLQ ILPALLFGGV EEQTVPEMPP SDNVARLIGK HFIDTLPPTP
541 GKQRPQKGCK VCRKRGIRRD TRYYCPKCPR NPGLCFKPCF EIYHTQLHY.
```

In some embodiments, the piggyBac or piggyBac-like transposase is a hyperactive variant of SEQ ID NO: 14517. In certain embodiments, the piggyBac or piggyBac-like transposase is an integration defective variant of SEQ ID NO: 14517. The piggyBac or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14518)
    1 MAKRFYSAEE AAAHCMAPSS EEFSGSDSEY VPPASESDSS TEESWCSSST VSALEEPMEV
  61 DEDVDDLEDQ EAGDRADAAA GGEPAWGPPC NFPPEIPPFT TVPGVKVDTS NFEPINFFQL
121 FMTEAILQDM VLYTNVYAEQ YLTQNPLPRY ARAHAWHPTD IAEMKRFVGL TLAMGLIKAN
181 SLESYWNTTT VLSIPVFSAT MSRNRYQLLL RFLHFNNNAT AVPPDQPDHD RLHKLRPLID
241 SLSERFAAVY TPCQNICIDE SLLLFKGRLR FRQYIPSKRA RYGIKFYKLC ESSSGYTSYF
301 LIYEGKDSKL DPPGCPPDLT VSGKIVWELI SPLLGQGFHL YVDNFYSSIP LFTALYCLDT
361 PACGTINRTR KGLPRALLDK KLNRGETYAL RKNELLAIKF FDKKNVFMLT SIHDESVIRE
421 QRVGRPPKNK PLCSKEYSKY MGGVDRTDQL QHYYNATRKT SAWYKKVGIY LIQMALRNSY
481 IVYKAAVPGP KLSYYKYQLQ ILPALLFGGV EEQTVPEMLP SDNVARLIGK HFIDTLPPTP
541 GKQRPQKGCK VCRKRGIRRD TRYYCPKCPR NPGLCFKPCF EIYHTQLHY.
```

In certain embodiments, the piggyBac or piggyBac-like transposase is isolated or derived from Xenopus tropicalis. In certain embodiments, the piggyBac or piggyBac-like transposase is a hyperactive piggyBac or piggyBac-like transposase. In certain embodiments, the hyperactive piggyBac or piggyBac-like transposase comprises a sequence at least 90% identical to:

```
(SEQ ID NO: 14572)
    1 MAKRFYSAEE AAAHCSASSS EEFSGSDSEY VPPASESDSS TEESWCSSST VSALEEPMEV
  61 DEDVDDLEDQ EAGDRADAAA GGEPAWGPPC NFPPEIPPFT TVPGVKVDTS NFEPINFFQL
121 FMTEAILQDM VLYTNVYAEQ YLTQNPLTRG ARAHAWHPTD IAEMKRFVGL TLAMGLIKAN
181 SIESYWDTTT VLSIPVFGAT MSRNRYQLLL RFLHFNNNAT AVPPDQPGHD RLHKLRPLID
241 SLSERFANVY TPCQNICIDE SLMLFKGRLQ FRQYIPSKRA RYGIKFYKLC ESSTGYTSYF
301 LIYEGKDSKL DPPGCPPDLT VSGKIVWELI SPLLGQGFHL YVDNFYSSIP LFTALYCLNT
361 PACGTINRNR KGLPRALLDK KLNRGETYAL RKNELLAIKF FDKKNVFMLT SIHDESVIRE
421 QRVGRPPKNK PLCSKEYSKY MGGVDRTDQL QHYYNATRKT RHWYKKVGIY LIQMALRNSY
481 IVYKAAVPGP KLSYYKYQLQ ILPALLFGGV EEQTVPEMPD SDNVARLIGK HFIDTLPPTP
541 GKQRPQKGCK VCRKRGIRRD TRYYCPKCPR NPGLCRKPCF EIYHTQLHY.
```

In certain embodiments, piggyBac or piggyBac-like transposase is a hyperactive piggyBac or piggyBac-like transposase. A hyperactive piggyBac or piggyBac-like transposase is a transposase that is more active than the naturally occurring variant from which it is derived. In certain embodiments, a hyperactive piggyBac or piggyBac-like transposase is more active than the transposase of SEQ ID NO: 14517. In certain embodiments, the hyperactive piggyBac or piggyBac-like transposase comprises a sequence of:

```
(SEQ ID NO: 14572)
    1 MAKRFYSAEE AAAHCSASSS EEFSGSDSEY VPPASESDSS TEESWCSSST VSALEEPMEV
  61 DEDVDDLEDQ EAGDRADAAA GGEPAWGPPC NFPPEIPPFT TVPGVKVDTS NFEPINFFQL
121 FMTEAILQDM VLYTNVYAEQ YLTQNPLTRG ARAHAWHPTD IAEMKRFVGL TLAMGLIKAN
181 SIESYWDTTT VLSIPVFGAT MSRNRYQLLL RFLHFNNNAT AVPPDQPGHD RLHKLRPLID
241 SLSERFANVY TPCQNICIDE SLMLFKGRLQ FRQYIPSKRA RYGIKFYKLC ESSTGYTSYF
301 LIYEGKDSKL DPPGCPPDLT VSGKIVWELI SPLLGQGFHL YVDNFYSSIP LFTALYCLNT
361 PACGTINRNR KGLPRALLDK KLNRGETYAL RKNELLAIKF FDKKNVFMLT SIHDESVIRE
421 QRVGRPPKNK PLCSKEYSKY MGGVDRTDQL QHYYNATRKT RHWYKKVGIY LIQMALRNSY
481 IVYKAAVPGP KLSYYKYQLQ ILPALLFGGV EEQTVPEMPD SDNVARLIGK HFIDTLPPTP
541 GKQRPQKGCK VCRKRGIRRD TRYYCPKCPR NPGLCRKPCF EIYHTQLHY.
```

In certain embodiments, the hyperactive piggyBac or piggyBac-like transposase comprises a sequence of:

```
(SEQ ID NO: 14624)
    1 MAKRFYSAEE AAAHCMASSS EEFSGSDSEY VPPASESDSS TEESWCSSST VSALEEPMEV
  61 DEDVDDLEDQ EAGDRADAAA GGEPAWGPPC NFPPEIPPFT TVPGVKVDTS NFEPINFFQL
121 FMTEAILQDM VLYTNVYAEQ YLTQNPLTRY ARAHAWHPTD IAEMKRFVGL TLAMGLIKAN
181 SLESYWDTTT VLSIPVFSAT MSRNRYQLLL RFLHFNNNAT AVPPDQPGHD RLHKLRPLID
241 SLSERFAAVY TPCQNICIDE SLLLFKGRLQ FRQYIPSKRA RYGIKFYKLC ESSSGYTSYF
301 LIYEGKDSKL DPPGCPPDLT VSGKIVWELI SPLLGQGFHL YVDNFYSSIP LFTALYCLNT
361 PACGTINRNR KGLPRALLDK KLNRGETYAL RKNELLAIKF FDKKNVFMLT SIHDESVIRE
421 QRVGRPPKNK PLCSKEYSKY MGGVDRTDQL QHYYNATRKT RHWYKKVGIY LIQMALRNSY
481 IVYKAAVPGP KLSYYKYQLQ ILPALLFGGV EEQTVPEMPP SDNVARLIGK HFIDTLPPTP
541 GKQRPQKGCK VCRKRGIRRD TRYYCPKCPR NPGLCRKPCF EIYHTQLHY.
```

In certain embodiments, the hyperactive piggyBac or piggyBac-like transposase comprises a sequence of:

```
(SEQ ID NO: 14625)
    1 MAKRFYSAEE AAAHCMASSS EEFSGSDSEY VPPASESDSS TEESWCSSST VSALEEPMEV
  61 DEDVDDLEDQ EAGDRADAAA GGEPAWGPPC NFPPEIPPFT TVPGVKVDTS NFEPINFFQL
121 FMTEAILQDM VLYTNVYAEQ YLTQNPLPRY ARAHAWHPTD IAEMKRFVGL TLAMGLIKAN
181 SLESYWDTTT VLKIPVFSAT MSRNRYQLLL RFLHFNNNAT AVPPDQPGHD RLHKLRPLID
241 SLSERFAAVY TPCQNICIDE SLLLFKGRLQ FRQYIPSKRA RYGIKFYKLC ESSSGYTSYF
301 LIYEGKDSKL DPPGCPPDLT VSGKIVWELI SPLLGQGFHL YVDNFYSSIP LFTALYCLNT
361 PACGTINRNR KGLPRALLDK KLNRGETYAL RKNELLAIKF FDKKNVFMLT SIHDESVIRE
421 QRVGRPPKNK PLCSKEYSKY MGGVDRTDQL QHYYNATRKT RHWYKKVGIY LIQMALRNSY
481 IVYKAAVPGP KLSYYKYQLQ ILPALLFGGV EEQTVPEMPP SDNVARLIGK HFIDTLPPTP
541 GKQRPQKGCK VCRKRGIRRD TRYYCPKCPR NPGLCFKPCF EIYHTQLHY.
```

In certain embodiments, the hyperactive piggyBac or piggyBac-like transposase comprises a sequence of:

```
(SEQ ID NO: 14627)
    1 MAKRFYSAEE AAAHCMASSS EQTSGSDSEY VPPASESDSS TEESWCSSST VSALEEPMEV
  61 DEDVDDLEDQ EAGDRADAAA GGEPAWGPPC NFPPEIPPFT TVPGVKVDTS NFEPINFFQL
121 FMTEAILQDM VLYTNVYAEQ YLTQNPLTRY ARAHAWHPTD IAEMKRFVGL TLAMGLIKAN
181 SIESYWDTTT VLSIPVFGAT MSRNRYQLLL RFLHFNNNAT AVPPDQPGHD RLHKLRPLID
241 SLSERFANVY TPCQNICIDE SLLLFKGRLQ FRQYIPSKRA RYGIKFYKLC ESSSGYTSYF
301 LIYEGKDSKL DPPGCPPDLT VSGKIVWELI SPLLGQGFHL YVDNFYSSIP LFTALYCLNT
361 PACGTINRNR KGLPRALLDK KLNRGETYAL RKNELLAIKF FDKKNVFMLT SIHDESVIRE
421 QRVGRKPKNK PLCSKEYSKY MGGVDRTDQL QHYYNATRKT RHWYKKVGIY LIQMALRNSY
481 IVYKAAVPGP KLSYYKYQLQ ILPALLFGGV EEQTVPEMPP SDNVARLIGK HFIDTLPPTP
541 GKQRPQKGCK VCRKRGIRRD TRYYCPKCPR NPGLCRKPCF EIYHTQLHY.
```

In certain embodiments, the hyperactive piggyBac or piggyBac-like transposase comprises a sequence of:

```
(SEQ ID NO: 14628)
    1 MAKRFYSAEE AAAHCSASSS EEFSGSDSEY VPPASESDSS TEESWCSSST VSALEEPMEV
  61 DEDVDDLEDQ EAGDRADAAA GGEPAWGPPC NFPPEIPPFT TVPGVKVDTS NFEPINFFQL
121 FMTEAILQDM VLYTNVYAEQ YLTQNPLTRG ARAHAWHPTD IAEMKRFVGL TLAMGLIKAN
181 SLESYWDTTT VLSIPVFGAT MSRNRYQLLL RFLHFNNNAT AVPPDQPGHD RLHKLRPLID
241 SLSERFANVY TPCQNICIDE SLMLFKGRLQ FRQYIPSKRA RYGIKFYKLC ESSTGYTSYF
301 LIYEGKDSKL DPPGCPPDLT VSGKIVWELI SPLLGQGFHL YVDNFYSSIP LFTALYCLNT
361 PACGTINRNR KGLPRALLDK KLNRGETYAL RKNELLAIKF FDKKNVFMLT SIHDESVIRE
421 QRVGRPPKNK PLCSKEYSKY MGGVDRTDQL QHYYNATRKT RHWYKKVGIY LIQMALRNSY
481 IVYKAAVPGP KLSYYKYQLQ ILPALLFGGV EEQTVPEMPP SDNVARLIGK HFIDTLPPTP
541 GKQRPQKGCK VCRKRGIRRD TRYYCPKCPR NPGLCRKPCF EIYHTQLHY.
```

In certain embodiments, the hyperactive piggyBac or piggyBac-like transposase comprises a sequence of:

```
(SEQ ID NO: 16820)
    1 MAKRFYSAEE AAAHCMASSS EEFSGSDSEY VPPASESDSS TEESWCSSST VSALEEPMEV
  61 DEDVDDLEDQ EAGDRADAAA GGEPAWGPPC NFPPEIPPFT TVPGVKVDTS NFEPINFFQL
121 FMTEAILQDM VLYTNVYAEQ YLTQNPLTRY ARAHAWHPTD IAEMKRFVGL TLAMGLIKAN
181 SLESYWDTTT VLSIPVFGAT MSRNRYQLLL RFLHFNNNAT AVPPDQPGHD RLHKLRPLID
241 SLSERFANVY TPCQNICIDE SLLLFKGRLQ FRQYIPSKRA RYGIKFYKLC ESSSGYTSYF
301 LIYEGKDSKL DPPGCPPDLT VSGKIVWELI SPLLGQGFHL YVDNFYSSIP LFTALYCLNT
361 PACGTINRNR KGLPRALLDK KLNRGETYAL RKNELLAIKF FDKKNVFMLT SIHDESVIRE
421 QRVGRPPKNK PLCSKEYSKY MGGVDRTDQL QHYYNATRKT RHWYKKVGIY LIQMALRNSY
481 IVYKAAVPGP KLSYYKYQLQ ILPALLFGGV EEQTVPEMPP SDNVARLIGK HFIDTLPPTP
541 GKQRPQKGCK VCRKRGIRRD TRYYCPKCPR NPGLCRKPCF EIYHTQLHY.
```

In certain embodiments, the hyperactive piggyBac or piggyBac-like transposase comprises an amino acid substitution at a position selected from amino acid 6, 7, 16, 19, 20, 21, 22, 23, 24, 26, 28, 31, 34, 67, 73, 76, 77, 88, 91, 141, 145, 146, 148, 150, 157, 162, 179, 182, 189, 192, 193, 196, 198, 200, 210, 212, 218, 248, 263, 270, 294, 297, 308, 310, 333, 336, 354, 357, 358, 359, 377, 423, 426, 428, 438, 447, 450, 462, 469, 472, 498, 502, 517, 520, 523, 533, 534, 576, 577, 582, 583 or 587 (relative to SEQ ID NO: 14517). In certain embodiments, the hyperactive piggyBac or piggyBac-like transposase comprises an amino acid substitution of Y6C, S7G, M16S, S19G, S20Q, S20G, S20D, E21D, E22Q, F23T, F23P, S24Y, S26V, S28Q, V31K, A34E, L67A, G73H, A76V, D77N, P88A, N91D, Y141Q, Y141A, N145E, N145V, P146T, P146V, P146K, P148T, P148H, Y150G, Y150S, Y150C, H157Y, A162C, A179K, L182I, L182V, T189G, L192H, S193N, S193K, V196I, S198G, T200W, L210H, F212N, N218E, A248N, L263M, Q270L, S294T, T297M, S308R, L310R, L333M, Q336M, A354H, C357V, L358F, D359N, L377I, V 423H, P426K, K428R, S438A, T447G, T447A, L450V, A462H, A462Q, I469V, I472L, Q498M, L502V, E517I, P520D, P520G, N523S, 1533E, D534A, F576R, F576E, K577I, I582R, Y583F, L587Y or L587W, or any combination thereof including at least 1, 2, 3, 4, 5, 6, 7, 8, 9, 10 or all of these mutations (relative to SEQ ID NO: 14517).

In certain embodiments, the hyperactive piggyBac or piggyBac-like transposase comprises one or more substitutions of an amino acid that is not wild type, wherein the one or more substitutions a for wild type amino acid comprises a substitution of A2X, K3X, R4X, F5X, Y6X, S7X, A11X, A13X, C15X, M16X, A17X, S18X, S19X, S20X, E21X, E22X, F23X, S24X, G25X, 26X, D27X, S28X, E29X, E42X, E43X, S44X, C46X, S47X, S48X, S49X, T50X, V51X, S52X, A53X, L54X, E55X, E56X, P57X, M58X, E59X, E62X, D63X, V64X, D65X, D66X, L67X, E68X, D69X, Q70X, E71X, A72X, G73X, D74X, R75X, A76X, D77X, A78X, A79X, A80X, G81X, G82X, E83X, P84X, A85X, W86X, G87X, P88X, P89X, C90X, N91X, F92X, P93X, E95X, I96X, P97X, P98X, F99X, T100X, T101X, P103X, G104X, V105X, K106X, V107X, D108X, T109X, N111X, P114X, I115X, N116X, F117X, F118X, Q119X, M122X, T123X, E124X, A125X, I126X, L127X, Q128X, D129X, M130X, L132X, Y133X, V126X, Y127X, A138X, E139X, Q140X, Y141X, L142X, Q144X, N145X, P146X, L147X, P148X, Y150X, A151X, A155X, H157X, P158X, I161X, A162X, V168X, T171X, L172X, A173X, M174X, I177X, A179X, L182X, D187X, T188X, T189X, T190X, L192X, S193X, I194X, P195X, V196X, S198X, A199X, T200X, S202X, L208X, L209X, L210X, R211X, F212X, F215X, N217X, N218X, A219X, T220X, A221X, V222X, P224X, D225X, Q226X, P227X, H229X, R231X, H233X, L235X, P237X, I239X, D240X, L242X, S243X, E244X, R244X, F246X, A247X, A248X, V249X, Y250X, T251X, P252X, C253X, Q254X, I256X, C257X, I258X, D259X, E260X, S261X, L262X, L263X, L264X, F265X, K266X, G267X, R268X, L269X, Q270X, F271X, R272X, Q273X, Y274X, I275X, P276X, S277X, K278X, R279X, A280X, R281X, Y282X, G283X, I284X, K285X, F286X, Y287X, K288X, L289X, C290X, E291X, S292X, S293XS294X, G295X, Y296X, T297X, S298X, Y299X, F300X, E304X, L310X, P313X, G314X, P316X, P317X, D318X, L319X, T320X, V321X, K324X, E328X, I330X, S331X, P332X, L333X, L334X, G335X, Q336X, F338X, L340X, D343X, N344X, F345X, Y346X, S347X, L351X, F352X, A354X, L355X, Y356X, C357X, L358X, D359X, T360X, R422X, Y423X, G424X, P426X, K428X, N429X, K430X, P431X, L432X, S434X, K435X, E436X, S438X, K439X, Y440X, G443X, R446X, T447X, L450X, Q451X, N455X, T460X, R461X, A462X, K465X, V467X, G468X, I469X, Y470X, L471X, I472X, M474X, A475X, L476X, R477X, S479X, Y480X, V482XY483X, K484X, A485X, A486X, V487X, P488X, P490X, K491X, S493X, Y494X, Y495X, K496X, Y497T, Q498X, L499X, Q500X, I501X, L502X, P503X, A504X, L505X, L506X, F507X, G508X, G509X, V510X, E511X, E512X, Q513X, T514X, V515X, E517X, M518X, P519X, P520X, S521X, D522X, N523X, V524X, A525X, L527X, I528X, K530X, H531X, F532X, I533X, D534X, T535X, L536X, T539X, P540X, Q546X, K550X, R553X, K554X, R555X, G556X, I557X, R558X, R559X, D560X, T561X, Y564X, P566X, K567X, P569X, R570X, N571X, L574X, C575X, F576X, K577X, P578X, F580X, E581X, I582X, Y583X, T585X, Q586X, L587X, H588X or Y589X (relative to SEQ ID NO: 14517). A list of hyperactive amino acid substitutions can be found in U.S. Pat. No. 10,041,077, the contents of which are incorporated by reference in their entirety.

In certain embodiments, the piggyBac or piggyBac-like transposase is integration deficient. In certain embodiments, an integration deficient piggyBac or piggyBac-like transposase is a transposase that can excise its corresponding transposon, but that integrates the excised transposon at a lower frequency than a corresponding naturally occurring transposase. In certain embodiments, the piggyBac or piggyBac-like transposase is an integration deficient variant of SEQ ID NO: 14517. In certain embodiments, the integration deficient piggyBac or piggyBac-like transposase is deficient relative to SEQ ID NO: 14517.

In certain embodiments, the piggyBac or piggyBac-like transposase is active for excision but deficient in integration. In certain embodiments, the integration deficient piggyBac or piggyBac-like transposase comprises a sequence that is at least 90% identical to a sequence of:

```
(SEQ ID NO: 14605)
    1 MAKRFYSAEE AAAHCMASSS EEFSGSDSEY VPPASESDSS TEESWCSSST VSALEEPMEV
  61 DEDVDDLEDQ EAGDRVDAAA GGEPAWGPPC NFPPEIPPFT TVPGVKVDTS NFEPINFFQL
121 FMTEAILQDM VLYTNVYAEQ YLTQNPLPRY ARAHAWHPTD IAEMKRFVGL TLAMGLIKAN
181 SLESYWDTTT VLSIPVFSAT MSRNRYQLLL KFLHFNNEAT AVPPDQPGHD RLHKLRPLID
241 SLSERFAAVY TPCQNICIDE SLLLFKGRLQ FRQYIPSKRA RYGIKFYKLC ESSSGYTSYF
301 LIYEGKDSKL DPPGCPPDLT VSGKIVWELI SPLLGQGFHL YVDNFYSSIP LFTALYCLDT
361 PACGTINRNR KGLPRALLDK KLNRGETYAL RKNELLAIKF FDKKNVFMLT SIHDESVIRE
421 QRVGRPPKNK PLCSKEYSKY MGGVDRTDQL QHYYNATRKT RAWYKKVGIY LIQMALRNSY
481 IVYKAAVPGP KLSYYKYQLQ ILPALLFGGV EEQTVPEMPP SDNVARLIGK HFIDTLPPTP
541 GKQRPQKGCK VCRKRGIRRD TRYYCPKCPR NPGLCFKPCF EIYHTQLHYG RR.
```

In certain embodiments, the integration deficient piggyBac or piggyBac-like transposase comprises a sequence that is at least 90% identical to a sequence of:

```
(SEQ ID NO: 14604)
    1 MAKRFYSAEE AAAHCMASSS EEFSGSDSEY VPPASESDSS TEESWCSSST VSALEEPMEV
  61 DEDVDDLEDQ EAGDRADAAA GGEPAWGPPC NFPPEIPPFT TVPGVKVDTS NFEPINFFQL
121 FMTEAILQDM VLYTNVYAEQ YLTQVPLPRY ARAHAWHPTD IAEMKRFVGL TLAMGLIKAN
181 SLESYWDTTT VLNIPVFSAT MSRNRYQLLL RFLEFNNEAT AVPPDQPGHD RLHKLRPLID
241 SLSERFAAVY TPCQNICIDE SLLLFKGRLQ FRQYIPSKRA RYGIKFYKLC ESSSGYTSYF
301 LIYEGKDSKL DPPGCPPDLT VSGKIVWELI SPLLGQGFHL YVDNFYSSIP LFTALYCLDT
361 PACGTINRNR KGLPRALLDK KLNRGETYAL RKNELLAIKF FDKKNVFMLT SIHDESVIRE
421 QRVGRPPKNK PLCSKEYSKY MGGVDRTDQL QHYYNATRKT RAWYKKVGIY LIQMALRNSY
481 IVYKAAVPGP KLSYYKYQLQ ILPALLFGGV EEQTVPEMPP SDNVARLIGK HFIDTLPPTP
541 GKQRPQKGCK VCRKRGIRRD TRYYCPKCPR NPGLCFKPCF EIYHTQLHY.
```

In certain embodiments, the integration deficient piggyBac or piggyBac-like transposase comprises a sequence that is at least 90% identical to a sequence of:

```
(SEQ ID NO: 14611)
    1 MAKRFYSAEE AAAHCMASSS EEFSGSDSEY VPPASESDSS TEESWCSSST VSALEEPMEV
  61 DEDVDDLEDQ EAGDRADAAA GGEPAWGPPC NFPPEIPPFT TVPGVKVDTS NFEPINFFQL
121 FMTEAILQDM VLYTNVYAEQ YLTQNVLPRY ARAHAWHPTD IAEMKRFVGL TLAMGLIKAN
181 SLESYWDTTT VLSIPVFSAT MSRNRYQLLL RFLHFNNDAT AVPPDQPGHD RLHKLRPLID
241 SLTERFAAVY TPCQNICIDE SLLLFKGRLQ FRQYIPSKRA RYGIKFYKLC ESSSGYTSYF
301 LIYEGKDSKL DPPGCPPDLT VSGKIVWELI SPLLGQGFHL YVDNFYSSIP LFTALYCLDT
361 PACGTINRNR KGLPRALLDK KLNRGETYAL RKNELLAIKF FDKKNVFMLT SIHDESVIRE
421 QRVGRPPKNK PLCSKEYSKY MGGVDRTDQL QHYYNATRKT RAWYKKVGIY LIQMALRNSY
481 IVYKAAVPGP KLSYYKYQLQ ILPALLFGGV EEQTVPEMPP SDNVARLIGK HFIDTLPPTP
541 GKQRPQKGCK VCRKRGIRRD TRYYCPKCPR NPGLCFKPCF EIYHTQLHYG RR.
```

In certain embodiments, the integration deficient piggyBac or piggyBac-like transposase comprises SEQ ID NO: 14611. In certain embodiments, the integration deficient piggyBac or piggyBac-like transposase comprises a sequence that is at least 90% identical to a sequence of:

```
(SEQ ID NO: 14612)
    1 MAKRFYSAEE AAAHCMASSS EEFSGSDSEY VPPASESDSS TEESWCSSST VSALEEPMEV
  61 DEDVDDLEDQ EAGDRADAAP GGEPAWGPPC NFPPEIPPFT TVPGVKVDTS NFEPINFFQL
121 FMTEAILQDM VLYTNVYAEQ YLTQVPLPRY ARAHAWHPTD IAEMKRFVGL TLAMGLIKAN
181 SLESYWDTTT VLSIPVFSAT MSRNRYQLLL RFLHFNNEAT AVPPDQPGHD RLHKLRPLID
241 SLSERFAAVY TPCQNICIDE SLLLFKGRLQ FRQYIPSKRA RYGIKFYKLC ESSSGYTSYF
301 LIYEGKDSKL DPPGCPPDLT VSGKIVWELI SPLLGQGFHL YVDNFYSSIP LFTALYCLDT
361 PACGTINRNR KGLPRALLDK KLNRGETYAL RKNELLAIKF FDKKNVFMLT SIHDESVIRE
421 QRVGRPPKNK PLCSKEYSKY MGGVDRTDQL QHYYNATRKT RAWYKKVGIY LIQMALRNSY
481 IVYKAAVPGP KLSYYKYQLQ ILPALLFGGV EEQTVPEMPP SDNVARLIGK HFIDTLPPTP
541 GKQRPQKGCK VCRKRGIRRD TRYYCPKCPR NPGLCFKPCF EIYHTQLHYG RR.
```

In certain embodiments, the integration deficient piggyBac or piggyBac-like transposase comprises SEQ ID NO: 14612. In certain embodiments, the integration deficient piggyBac or piggyBac-like transposase comprises a sequence that is at least 90% identical to a sequence of:

```
(SEQ ID NO: 14613)
    1 MAKRFYSAEE AAAHCMASSS EEFSGSDSEY VPPASESDSS TEESWCSSST VSALEEPMEV
  61 DEDVDDLEDQ EAGDRADAAA GGEPAWGPPC NFPPEIPPFT TVPGVKVDTS NFEPINFFQL
121 FMTEAILQDM VLYTNVYAEQ YLTQVPLPRY ARAHAWHPTD IAEMKRFVGL TLAMGLIKAN
181 SLESYWDTTT VLNIPVFSAT MSRNRYQLLL RFLEFNNNAT AVPPDQPGHD RLHKLRPLID
241 SLSERFAAVY TPCQNICIDE SLLLFKGRLQ FRQYIPSKRA RYGIKFYKLC ESSSGYTSYF
301 LIYEGKDSKL DPPGCPPDLT VSGKIVWELI SPLLGQGFHL YVDNFYSSIP LFTALYCLDT
361 PACGTINRNR KGLPRALLDK KLNRGETYAL RKNELLAIKF FDKKNVFMLT SIHDESVIRE
421 QRVGRPPKNK PLCSKEYSKY MGGVDRTDQL QHYYNATRKT RAWYKKVGIY LIQMALRNSY
481 IVYKAAVPGP KLSYYKYQLQ ILPALLFGGV EEQTVPEMPP SDNVARLIGK HFIDTLPPTP
541 GKQRPQKGCK VCRKRGIRRD TRYYCPKCPR NPGLCFKPCF EIYHTQLHYG RR.
```

In certain embodiments, the integration deficient piggyBac or piggyBac-like transposase comprises SEQ ID NO: 14613. In certain embodiments, the integration deficient piggyBac or piggyBac-like transposase comprises an amino acid substitution wherein the Asn at position 218 is replaced by a Glu or an Asp (N218D or N218E) (relative to SEQ ID NO: 14517).

In certain embodiments, the excision competent, integration deficient piggyBac or piggyBac-like transposase comprises one or more substitutions of an amino acid that is not wild type, wherein the one or more substitutions a for wild type amino acid comprises a substitution of A2X, K3X, R4X, F5X, Y6X, S7X, ABX, E9X, E10X, A11X, A12X, A13X, H14X, C15X, M16X, A17X, S18X, S19X, S20X, E21X, E22X, F23X, S24X, G25X, 26X, D27X, S28X, E29X, V31X, P32X, P33X, A34X, S35X, E36X, S37X, D38X, S39X, S40X, T41X, E42X, E43X, S44X, W45X, C46X, S47X, S48X, S49X, T50X, V51X, S52X, A53X, L54X, E55X, E56X, P57X, M58X, E59X, V60X, M122X, T123X, E124X, A125X, L127X, Q128X, D129X, L132X, Y133X, V126X, Y127X, E139X, Q140X, Y141X, L142X, T143X, Q144X, N145X, P146X, L147X, P148X, R149X, Y150X, A151X, H154X, H157X, P158X, T159X, D160X, I161X, A162X, E163X, M164X, K165X, R166X, F167X, V168X, G169X, L170X, T171X, L172X, A173X, M174X, G175X, L176X, I177X, K178X, A179X, N180X, S181X, L182X, S184X, Y185X, D187X, T188X, T189X, T190X, V191X, L192X, S193X, I194X, P195X, V196X, F197X, S198X, A199X, T200X, M201X, S202X, R203X, N204X, R205X, Y206X, Q207X, L208X, L209X, L210X, R211X, F212X, L213X, H241X, F215X, N216X, N217X, N218X, A219X, T220X, A221X, V222X, P223X, P224X, D225X, Q226X, P227X, G228X, H229X, D230X, R231X, H233X, K234X, L235X, R236X, L238X, I239X, D240X, L242X, S243X, E244X, R244X, F246X, A247X, A248X, V249X, Y250X, T251X, P252X, C253X, Q254X, N255X, I256X, C257X, I258X, D259X, E260X, S261X, L262X, L263X, L264X, F265X, K266X, G267X, R268X, L269X, Q270X, F271X, R272X, Q273X, Y274X, I275X, P276X, S277X, K278X, R279X, A280X, R281X, Y282X, G283X, I284X, K285X, F286X, Y287X, K288X, L289X, C290X, E291X, S292X, S293X, S294X, G295X, Y296X, T297X, S298X, Y299X, F300X, I302X, E304X, G305X, K306X, D307X, S308X, K309X, L310X, D311X, P312X, P313X, G314X, C315X, P316X, P317X, D318X, L319X, T320X, V321X, S322X, G323X, K324X, I325X, V326X, W327X, E328X, L329X, I330X, S331X, P332X, L333X, L334X, G335X, Q336X, F338X, H339X, L340X, V342X, N344X, F345X, Y346X, S347X, S348X, I349X, L351X, T353X, A354X, Y356X, C357X, L358X, D359X, T360X, P361X, A362X, C363X, G364X, I366X, N367X, R368X, D369X, K371X, G372X, L373X, R375X, A376X, L377X, L378X, D379X, K380X, K381X, L382X, N383X, R384XG385X, T387X, Y388X, A389X, L390X, K392X, N393X, E394X, A397X, K399X, F400X, F401X, D402X, N405X, L406X, L409X, R422X, Y423X, G424X, E425X, P426X, K428X, N429X, K430X, P431X, L432X, S434X, K435X, E436X, S438X, K439X, Y440X, G442X, G443X, V444X, R446X, T447X, L450X, Q451X, H452X, N455X, T457X, R458X, T460X, R461X, A462X, Y464X, K465X, V467X, G468X, I469X, L471X, I472X, Q473X, M474X, L476X, R477X, N478X, S479X, Y480X, V482XY483X, K484X, A485X, A486X, V487X, P488X, G489X, P490X, K491X, L492X, S493X, Y494X, Y495X, K496X, Q498X, L499X, Q500X, I501X, L502X, P503X, A504X, L505X, L506X, F507X, G508X, G509X, V510X, E511X, E512X, Q513X, T514X, V515X, E517X, M518X, P519X, P520X, S521X, D522X, N523X, V524X, A525X, L527X, I528X, G529X, K530X, F532X, I533X, D534X, T535X, L536X, P537X, P538X, T539X, P540X, G541X, F542X, Q543X, R544X, P545X, Q546X, K547X, G548X, C549X, K550X, V551X, C552X, R553X, K554X, R555X, G556X, I557X, R558X, R559X, D560X, T561X, R562X, Y563X, Y564X, C565X, P566X, K567X, C568X, P569X, R570X, N571X, P572X, G573X, L574X, C575X, F576X, K577X, P578X, C579X, F580X, E581X, I582X, Y583X, H584X, T585X, Q586X, L587X, H588X or Y589X (relative to SEQ ID NO: 14517). A list of excision competent, integration deficient amino acid substitutions can be found in U.S. Pat. No. 10,041,077, the contents of which are incorporated by reference in their entirety.

In certain embodiments, the piggyBac or piggyBac-like transposase is fused to a nuclear localization signal. In certain embodiments, SEQ ID NO: 14517 or SEQ ID NO: 14518 is fused to a nuclear localization signal. In certain embodiments, the amino acid sequence of the piggyBac or piggyBac like transposase fused to a nuclear localization signal is encoded by a polynucleotide sequence comprising:

```
(SEQ ID NO: 14626)
      1 atggcaccca aaaagaaacg taaagtgatg gccaaaagat tttacagcgc cgaagaagca
    61 gcagcacatt gcatggcatc gtcatccgaa gaattctcgg ggagcgattc cgaatatgtc
  121 ccaccggcct cggaaagcga ttcgagcact gaggagtcgt ggtgttcctc ctcaactgtc
  181 tcggctcttg aggagccgat ggaagtggat gaggatgtgg acgacttgga ggaccaggaa
  241 gccggagaca gggccgacgc tgccgcggga ggggagccgg cgtggggacc tccatgcaat
  301 tttcctcccg aaatcccacc gttcactact gtgccgggag tgaaggtcga cacgtccaac
  361 ttcgaaccga tcaatttctt tcaactcttc atgactgaag cgatcctgca agatatggtg
  421 ctctacacta atgtgtacgc cgagcagtac ctgactcaaa acccgctgcc tcgctacgcg
  481 agagcgcatg cgtggcaccc gaccgatatc gcggagatga agcggttcgt gggactgacc
  541 ctcgcaatgg gcctgatcaa ggccaacagc ctcgagtcat actgggatac cacgactgtg
  601 cttagcattc cggtgttctc cgctaccatg tcccgtaacc gctaccaact cctgctgcgg
  661 ttcctccact tcaacaacaa tgcgaccgct gtgccacctg accagccagg acacgacaga
  721 ctccacaagc tgcggccatt gatcgactcg ctgagcgagc gattcgccgc ggtgtacacc
  781 ccttgccaaa acatttgcat cgacgagtcg cttctgctgt ttaaaggccg gcttcagttc
  841 cgccagtaca tcccatcgaa gcgcgctcgc tatggtatca aattctacaa actctgcgag
  901 tcgtccagcg gctacacgtc atacttcttg atctacgagg ggaaggactc taagctggac
  961 ccaccggggt gtccaccgga tcttactgtc tccggaaaaa tcgtgtggga actcatctca
1021 cctctcctcg gacaaggctt tcatctctac gtcgacaatt tctactcatc gatccctctg
1081 ttcaccgccc tctactgcct ggatactcca gcctgtggga ccattaacag aaaccggaag
1141 ggtctgccga gagcactgct ggataagaag ttgaacaggg gagagactta cgcgctgaga
1201 aagaacgaac tcctcgccat caaattcttc gacaagaaaa atgtgtttat gctcacctcc
1261 atccacgacg aatccgtcat ccgggagcag cgcgtgggca ggccgccgaa aaacaagccg
1321 ctgtgctcta aggaatactc caagtacatg gggggtgtcg accggaccga tcagctgcag
1381 cattactaca acgccactag aaagacccgg gcctggtaca agaaagtcgg catctacctg
1441 atccaaatgg cactgaggaa ttcgtatatt gtctacaagg ctgccgttcc gggcccgaaa
1501 ctgtcatact acaagtacca gcttcaaatc ctgccggcgc tgctgttcgg tggagtggaa
1561 gaacagactg tgcccgagat gccgccatcc gacaacgtgg cccggttgat cggaaagcac
1621 ttcattgata ccctgcctcc gacgcctgga aagcagcggc cacagaaggg atgcaaagtt
1681 tgccgcaagc gcggaatacg gcgcgatacc cgctactatt gcccgaagtg cccccgcaat
1741 cccggactgt gtttcaagcc ctgttttgaa atctaccaca cccagttgca ttac.
```

In certain embodiments, the piggyBac or piggyBac-like transposon is isolated or derived from Xenopus tropicalis. In certain embodiments, the piggyBac or piggyBac-like transposon comprises a sequence of:

```
(SEQ ID NO: 14519)
    1 ttaacctttt tactgccaat gacgcatggg atacgtcgtg gcagtaaaag ggcttaaatg
  61 ccaacgacgc gtcccatacg ttgttggcat tttaagtctt ctctctgcag cggcagcatg
121 tgccgccgct gcagagagtt tctagcgatg acagcccctc tgggcaacga gccggggggg
181 ctgtc.
```

In certain embodiments, the piggyBac or piggyBac-like transposon comprises a sequence of:

```
(SEQ ID NO: 14520)
    1 tttgcatttt tagacattta gaagcctata tcttgttaca gaattggaat tacacaaaaa
  61 ttctaccata ttttgaaagc ttaggttgtt ctgaaaaaaa caatatattg ttttcctggg
121 taaactaaaa gtcccctcga ggaaaggccc ctaaagtgaa acagtgcaaa acgttcaaaa
181 actgtctggc aatacaagtt ccactttgac caaaacggct ggcagtaaaa gggttaa.
```

In certain embodiments, the piggyBac or piggyBac-like transposon comprises SEQ ID NO: 14519 and SEQ ID NO: 14520. In certain embodiments, the piggyBac or piggyBac-like transposon comprises a sequence of:

```
(SEQ ID NO: 14521)
    1 ttaacccttt gcctgccaat cacgcatggg atacgtcgtg gcagtaaaag ggcttaaatg
  61 ccaacgacgc gtcccatacg ttgttggcat tttaagtctt ctctctgcag cggcagcatg
121 tgccgccgct gcagagagtt tctagcgatg acagcccctc tgggcaacga gccggggggg
181 ctgtc.
```

In certain embodiments, the piggyBac or piggyBac-like transposon comprises a sequence of:

```
(SEQ ID NO: 14522)
    1 tttgcatttt tagacattta gaagcctata tcttgttaca gaattggaat tacacaaaaa
  61 ttctaccata ttttgaaagc ttaggttgtt ctgaaaaaaa caatatattg ttttcctggg
121 taaactaaaa gtcccctcga ggaaaggccc ctaaagtgaa acagtgcaaa acgttcaaaa
181 actgtctggc aatacaagtt ccactttggg acaaatcggc tggcagtgaa agggttaa.
```

In certain embodiments, the piggyBac or piggyBac-like transposon comprises a sequence of:

```
(SEQ ID NO: 14523)
    1 ttaacctttt tactgccaat gacgcatggg atacgtcgtg gcagtaaaag ggcttaaatg
  61 ccaacgacgc gtcccatacg ttgttggcat tttaattctt ctctctgcag cggcagcatg
121 tgccgccgct gcagagagtt tctagcgatg acagcccctc tgggcaacga gccggggggg
181 ctgtc.
```

In certain embodiments, the piggyBac or piggyBac-like transposon comprises SEQ ID NO: 14520 and SEQ ID NO: 14519, SEQ ID NO: 14521 or SEQ ID NO: 14523. In certain embodiments, the piggyBac or piggyBac-like transposon comprises SEQ ID NO: 14522 and SEQ ID NO: 14519, SEQ ID NO: 14521 or SEQ ID NO: 14523. In certain embodiments, the piggyBac or piggyBac-like transposon comprises one end comprising at least 14, 16, 18, 20, 30 or 40 contiguous nucleotides from SEQ ID NO: 14519, SEQ ID NO: 14521 or SEQ ID NO: 14523. In certain embodiments, the piggyBac or piggyBac-like transposon comprises one end comprising at least 14, 16, 18, 20, 30 or 40 contiguous nucleotides from SEQ ID NO: 14520 or SEQ ID NO: 14522. In certain embodiments, the piggyBac or piggyBac-like transposon comprises one end with at least 90% identity to SEQ ID NO: 14519, SEQ ID NO: 14521 or SEQ ID NO: 14523. In certain embodiments, the piggyBac or piggyBac-like transposon comprises one end with at least 90% identity to SEQ ID NO: 14520 or SEQ ID NO: 14522. In one embodiment, one transposon end is at least 90% identical to SEQ ID NO: 14519 and the other transposon end is at least 90% identical to SEQ ID NO: 14520.

In certain embodiments, the piggyBac or piggyBac-like transposon comprises a sequence of TTAACCTTTTTACTGCCA (SEQ ID NO: 14524). In certain embodiments, the piggyBac or piggyBac-like transposon comprises a sequence of TTAACCCTTTGCCTGCCA (SEQ ID NO: 14526). In certain embodiments, the piggyBac or piggyBac-like transposon comprises a sequence of TTAACCYTTTTACTGCCA (SEQ ID NO: 14527). In certain embodiments, the piggyBac or piggyBac-like transposon comprises a sequence of TGGCAGTAAAAGGGTTAA (SEQ ID NO: 14529). In certain embodiments, the piggyBac or piggyBac-like transposon comprises a sequence of TGGCAGTGAAAGGGTTAA (SEQ ID NO: 14531). In certain embodiments, the piggyBac or piggyBac-like transposon comprises a sequence of TTAACCYTTTKMCTGCCA (SEQ ID NO: 14533). In certain embodiments, one end of the piggyBac or piggyBac-like transposon comprises a sequence selected from SEQ ID NO: 14524, SEQ ID NO: 14526 and SEQ ID NO: 14527. In certain embodiments, one end of the piggyBac™ (PB) or piggyBac-like transposon comprises a sequence selected from SEQ ID NO: 14529 and SEQ ID NO: 14531. In certain embodiments, each inverted terminal repeat of the piggyBac or piggyBac-like transposon comprises a sequence of ITR sequence of CCYTTTKMCTGCCA (SEQ ID NO: 14563). In certain embodiments, each end of the piggyBac™ (PB) or piggyBac-like transposon comprises SEQ ID NO: 14563 in inverted orientations. In certain embodiments, one ITR of the piggyBac or piggyBac-like transposon comprises a sequence selected from SEQ ID NO: 14524, SEQ ID NO: 14526 and SEQ ID NO: 14527. In certain embodiments, one ITR of the piggyBac or piggyBac-like transposon comprises a sequence selected from SEQ ID NO: 14529 and SEQ ID NO: 14531. In certain embodiments, the piggyBac or piggyBac like transposon comprises SEQ ID NO: 14533 in inverted orientation in the two transposon ends.

In certain embodiments, The piggyBac or piggyBac-like transposon may have ends comprising SEQ ID NO: 14519 and SEQ ID NO: 14520 or a variant of either or both of these having at least 90% sequence identity to SEQ ID NO: 14519 or SEQ ID NO: 14520, and the piggyBac or piggyBac-like transposase has the sequence of SEQ ID NO: 14517 or a variant showing at least %, 10%, 15%, 20%, 25%, 30%, 35%, 40%, 45%, 50%, 55%, 60%, 65%, 70%, 75%, 80%, 85%, 90%, 95%, 99% or any percentage in between sequence identity to SEQ ID NO: 14517 or SEQ ID NO: 14518. In certain embodiments, one piggyBac or piggyBac-like transposon end comprises at least 14 contiguous nucleotides from SEQ ID NO: 14519, SEQ ID NO: 14521 or SEQ ID NO: 14523, and the other transposon end comprises at least 14 contiguous nucleotides from SEQ ID NO: 14520 or SEQ ID NO: 14522. In certain embodiments, one transposon end comprises at least 15, at least 16, at least 17, at least 18, at least 19, at least 20, at least 22, at least 25, at least 30 contiguous nucleotides from SEQ ID NO: 14519, SEQ ID NO: 14521 or SEQ ID NO: 14523, and the other transposon end comprises at least 15, at least 16, at least 17, at least 18, at least 19, at least 20, at least 22, at least 25 or at least 30 contiguous nucleotides from SEQ ID NO: 14520 or SEQ ID NO: 14522.

In certain embodiments, the piggyBac or piggyBac-like transposase recognizes a transposon end with a 5′ sequence corresponding to SEQ ID NO: 14519, and a 3′ sequence corresponding to SEQ ID NO: 14520. It will excise the transposon from one DNA molecule by cutting the DNA at the 5′-TTAA-3′ sequence at the 5′ end of one transposon end to the 5′-TTAA-3′ at the 3′ end of the second transposon end, including any heterologous DNA that is placed between them, and insert the excised sequence into a second DNA molecule. In certain embodiments, truncated and modified versions of the 5′ and 3′ transposon ends will also function as part of a transposon that can be transposed by the piggyBac or piggyBac-like transposase. For example, the 5′ transposon end can be replaced by a sequence corresponding to SEQ ID NO: 14521 or SEQ ID NO: 14523, the 3′ transposon end can be replaced by a shorter sequence corresponding to SEQ ID NO: 14522. In certain embodiments, the 5′ and 3′ transposon ends share an 18 bp almost perfectly repeated sequence at their ends (5′-TTAACCYTTTKMCTGCCA: SEQ ID NO: 14533) that includes the 5′-TTAA-3′ insertion site, which sequence is inverted in the orientation in the two ends. That is in SEQ ID NO: 14519 and SEQ ID NO: 14523 the 5′ transposon end begins with the sequence 5′-TTAACCTTTTTACTGCCA-3′ (SEQ ID NO: 14524), or in SEQ ID NO: 14521 the 5′ transposon end begins with the sequence 5′-TTAACCCTTTGCCTGCCA-3′ (SEQ ID NO: 14526); the 3′ transposon ends with approximately the reverse complement of this sequence: in SEQ ID NO: 14520 it ends 5′ TGGCAGTAAAAGGGTTAA-3′ (SEQ ID NO: 14529), in SEQ ID NO: 14522 it ends 5′-TGGCAGTGAAAGGGTTAA-3′ (SEQ ID NO: 14531.) One embodiment of the invention is a transposon that comprises a heterologous polynucleotide inserted between two transposon ends each comprising SEQ ID NO: 14533 in inverted orientations in the two transposon ends. In certain embodiments, one transposon end comprises a sequence selected from SEQ ID NOS: 14524, SEQ ID NO: 14526 and SEQ ID NO: 14527. In some embodiments, one transposon end comprises a sequence selected from SEQ ID NO: 14529 and SEQ ID NO: 14531.

In certain embodiments, the piggyBac™ (PB) or piggyBac-like transposon is isolated or derived from Xenopus tropicalis. In certain embodiments, the piggyBac or piggyBac-like transposon comprises at a sequence of:

(SEQ ID NO: 14573)
1 ccctttgcct gccaatcacg catgggatac gtcgtggcag taaaagggct taaatgccaa
61 cgacgcgtcc catacgtt.

In certain embodiments, the piggyBac or piggyBac-like transposon comprises at a sequence of:

(SEQ ID NO: 14574)
1 cctgggtaaa ctaaaagtcc cctcgaggaa aggcccctaa agtgaaacag tgcaaaacgt
61 tcaaaaactg tctggcaata caagttccac tttgggacaa atcggctggc agtgaaaggg.

In certain embodiments, the piggyBac or piggyBac-like transposon comprises at least 16 contiguous bases from SEQ ID NO: 14573 or SEQ ID NO: 14574, and inverted terminal repeat of

(SEQ ID NO: 14575)
CCYTTTBMCTGCCA.

In certain embodiments, the piggyBac or piggyBac-like transposon comprises at a sequence of:

```
(SEQ ID NO: 14579)
    1 ccctttgcct gccaatcacg catgggatac gtcgtggcag taaaagggct taaatgccaa
  61 cgacgcgtcc catacgttgt tggcatttta agtcttctct ctgcagcggc agcatgtgcc
121 gccgctgcag agagtttcta gcgatgacag cccctctggg caacgagccg ggggggctgt
181 c.
```

In certain embodiments, the piggyBac or piggyBac-like transposon comprises at a sequence of:

```
(SEQ ID NO: 14580)
    1 cctttttact gccaatgacg catgggatac gtcgtggcag taaaagggct taaatgccaa
  61 cgacgcgtcc catacgttgt tggcatttta attcttctct ctgcagcggc agcatgtgcc
121 gccgctgcag agagtttcta gcgatgacag cccctctggg caacgagccg ggggggctgt
181 c.
```

In certain embodiments, the piggyBac or piggyBac-like transposon comprises at a sequence of:

```
(SEQ ID NO: 14581)
    1 cctttttact gccaatgacg catgggatac gtcgtggcag taaaagggct taaatgccaa
  61 cgacgcgtcc catacgttgt tggcatttta agtcttctct ctgcagcggc agcatgtgcc
121 gccgctgcag agagtttcta gcgatgacag cccctctggg caacgagccg ggggggctgt
181 c.
```

In certain embodiments, the piggyBac or piggyBac-like transposon comprises at a sequence of:

```
(SEQ ID NO: 14582)
    1 cctttttact gccaatgacg catgggatac gtcgtggcag taaaagggct taaatgccaa
  61 cgacgcgtcc catacgttgt tggcatttta agtcttctct ctgcagcggc agcatgtgcc
121 gccgctgcag agag.
```

In certain embodiments, the piggyBac or piggyBac-like transposon comprises at a sequence of:

(SEQ ID NO: 14583)
1 cctttttact gccaatgacg catgggatac gtcgtggcag taaaagggct taaatgccaa
61 cgacgcgtcc catacgttgt tggcatttta agtctt.

In certain embodiments, the piggyBac or piggyBac-like transposon comprises at a sequence of:

(SEQ ID NO: 14584)
1 ccctttgcct gccaatcacg catgggatac gtcgtggcag taaaagggct taaatgccaa
61 cgacgcgtcc catacgttgt tggcatttta agtctt.

In certain embodiments, the piggyBac or piggyBac-like transposon comprises at a sequence of:

```
(SEQ ID NO: 14585)
    1 ttatcctttt tactgccaat gacgcatggg atacgtcgtg gcagtaaaag ggcttaaatg
  61 ccaacgacgc gtcccatacg ttgttggcat tttaagtctt ctctctgcag cggcagcatg
121 tgccgccgct gcagagagtt tctagcgatg acagcccctc tgggcaacga gccggggggg
181 ctgtc.
```

In certain embodiments, the piggyBac or piggyBac-like transposon comprises at a sequence of:

```
(SEQ ID NO: 14586)
    1 tttgcatttt tagacattta gaagcctata tcttgttaca gaattggaat tacacaaaaa
  61 ttctaccata ttttgaaagc ttaggttgtt ctgaaaaaaa caatatattg ttttcctggg
121 taaactaaaa gtcccctcga ggaaaggccc ctaaagtgaa acagtgcaaa acgttcaaaa
181 actgtctggc aatacaagtt ccactttggg acaaatcggc tggcagtgaa aggg.
```

In certain embodiments, the piggyBac or piggyBac-like transposon comprises a 5′ transposon end sequence selected from SEQ ID NO: 14573 and SEQ ID NOs: 14579-14585. In certain embodiments, the 5′ transposon end sequence is preceded by a 5′ target sequence. In certain embodiments, the piggyBac or piggyBac-like transposon comprises at a sequence of:

```
(SEQ ID NO: 14587)
    1 tttgcatttt tagacattta gaagcctata tcttgttaca gaattggaat tacacaaaaa
  61 ttctaccata ttttgaaagc ttaggttgtt ctgaaaaaaa caatatattg ttttcctggg
121 taaactaaaa gtcccctcga ggaaaggccc ctaaagtgaa acagtgcaaa acgttcaaaa
181 actgtctggc aatacaagtt ccactttgac caaaacggct ggcagtaaaa ggg.
```

In certain embodiments, the piggyBac or piggyBac-like transposon comprises at a sequence of:

```
(SEQ ID NO: 14588)
    1 ttgttctgaa aaaaacaata tattgttttc ctgggtaaac taaaagtccc ctcgaggaaa
  61 ggcccctaaa gtgaaacagt gcaaaacgtt caaaaactgt ctggcaatac aagttccact
121 ttgaccaaaa cggctggcag taaaaggg.
```

In certain embodiments, the piggyBac or piggyBac-like transposon comprises at a sequence of:

```
(SEQ ID NO: 14589)
    1 tttgcatttt tagacattta gaagcctata tcttgttaca gaattggaat tacacaaaaa
  61 ttctaccata ttttgaaagc ttaggttgtt ctgaaaaaaa caatatattg ttttcctggg
121 taaactaaaa gtcccctcga ggaaaggccc ctaaagtgaa acagtgcaaa acgttcaaaa
181 actgtctggc aatacaagtt ccactttgac caaaacggct ggcagtaaaa gggttat.
```

In certain embodiments, the piggyBac or piggyBac-like transposon comprises at a sequence of:

```
(SEQ ID NO: 14590)
    1 ttgttctgaa aaaaacaata tattgttttc ctgggtaaac taaaagtccc ctcgaggaaa
  61 ggcccctaaa gtgaaacagt gcaaaacgtt caaaaactgt ctggcaatac aagttccact
121 ttgggacaaa tcggctggca gtgaaaggg.
```

In certain embodiments, the piggyBac or piggyBac-like transposon comprises a 3′ transposon end sequence selected from SEQ ID NO: 14574 and SEQ ID NOs: 14587-14590. In certain embodiments, the 3′ transposon end sequence is followed by a 3′ target sequence. In certain embodiments, the 5′ and 3′ transposon ends share a 14 repeated sequence inverted in orientation in the two ends (SEQ ID NO: 14575) adjacent to the target sequence. In certain embodiments, the piggyBac or piggyBac-like transposon comprises a 5′ transposon end comprising a target sequence and a sequence that is selected from SEQ ID NOs: 14582-14584 and 14573, and a 3′ transposon end comprising a sequence selected from SEQ ID NOs: 14588-14590 and 14574 followed by a 3′ target sequence.

In certain embodiments, the 5′ transposon end of the piggyBac or piggyBac-like transposon comprises

1 atcacgcatg ggatacgtcg tggcagtaaa agggcttaaa tgccaacgac gcgtcccata
61 cgtt

(SEQ ID NO: 14591), and an ITR. In certain embodiments, the 5′ transposon end comprises

1 atgacgcatg ggatacgtcg tggcagtaaa agggcttaaa tgccaacgac gcgtcccata
61 cgttgttggc attttaagtc tt

(SEQ ID NO: 14592) and an ITR. In certain embodiments, the 3′ transposon end of the piggyBac or piggyBac-like transposon comprises

1 cctgggtaaa ctaaaagtcc cctcgaggaa aggcccctaa agtgaaacag tgcaaaacgt
61 tcaaaaactg tctggcaata caagttccac tttgggacaa atcggc

(SEQ ID NO: 14593) and an ITR. In certain embodiments, the 3′ transposon end comprises

```
    1 ttgttctgaa aaaaacaata tattgttttc ctgggtaaac taaaagtccc ctcgaggaaa
  61 ggcccctaaa gtgaaacagt gcaaaacgtt caaaaactgt ctggcaatac aagttccact
121 ttgaccaaaa cggc
```

### (SEQ ID NO: 14594) and an ITR.

In certain embodiments, one transposon end comprises a sequence that is at least 90%, at least 95%, at least 99% or any percentage in between identical to SEQ ID NO: 14573 and the other transposon end comprises a sequence that is at least 90%, at least 95%, at least 99% or any percentage in between identical to SEQ ID NO: 14574. In certain embodiments, one transposon end comprises at least 14, at least 16, at least 18, at least 20 or at least 25 contiguous nucleotides from SEQ ID NO: 14573 and one transposon end comprises at least 14, at least 16, at least 18, at least 20 or at least 25 contiguous nucleotides from SEQ ID NO: 14574. In certain embodiments, one transposon end comprises at least 14, at least 16, at least 18, at least 20 from SEQ ID NO: 14591, and the other end comprises at least 14, at least 16, at least 18, at least 20 from SEQ ID NO: 14593. In certain embodiments, each transposon end comprises SEQ ID NO: 14575 in inverted orientations.

In certain embodiments, the piggyBac or piggyBac-like transposon comprises a sequence selected from of SEQ ID NO: 14573, SEQ ID NO: 14579, SEQ ID NO: 14581, SEQ ID NO: 14582, SEQ ID NO: 14583, and SEQ ID NO: 14588, and a sequence selected from SEQ ID NO: 14587, SEQ ID NO: 14588, SEQ ID NO: 14589 and SEQ ID NO: 14586 and the piggyBac or piggyBac-like transposase comprises SEQ ID NO: 14517 or SEQ ID NO: 14518.

In certain embodiments, the piggyBac or piggyBac-like transposon comprises ITRs of CCCTTTGCCTGCCA (SEQ ID NO: 14622) (5′ ITR) and TGGCAGTGAAAGGG (SEQ ID NO: 14623) (3′ ITR) adjacent to the target sequences.

In certain embodiments of the methods of the disclosure, the transposase enzyme is a piggyBac or piggyBac-like transposase enzyme. In certain embodiments, the piggyBac or piggyBac-like transposase enzyme is isolated or derived from Helicoverpa armigera. The piggyBac or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14525)
    1 MASRQRLNHD EIATILENDD DYSPLDSESE KEDCVVEDDV WSDNEDAIVD FVEDTSAQED
  61 PDNNIASRES PNLEVTSLTS HRIITLPQRS IRGKNNHVWS TTKGRTTGRT SAINIIRTNR
121 GPTRMCRNIV DPLLCFQLFI TDEIIHEIVK WTNVEIIVKR QNLKDISASY RDINTMEIWA
181 LVGILTLTAV MKDNHLSTDE LFDATFSGTR YVSVMSRERF EFLIRCIRMD DKTLRPTLRS
241 DDAFLPVRKI WEIFINQCRQ NHVPGSNLTV DEQLLGFRGR CPFRMYIPNK PDKYGIKFPM
301 MCAAATKYMI DAIPYLGKST KTNGLPLGEF YVKDLTKTVH GTNRNITCDN WFTSIPLAKN
361 MLQAPYNLTI VGTIRSNKRE MPEEIKNSRS RPVGSSMFCF DGPLTLVSYK PKPSKMVFLL
421 SSCDENAVIN ESNGKPDMIL FYNQTKGGVD SFDQMCKSMS ANRKTNRWPM AVFYGMLNMA
481 FVNSYIIYCH NKINKQEKPI SRKEFMKKLS IQLTTPWMQE RLQAPTLKRT LRDNITNVLK
541 NVVPASSENI SNEPEPKKRR YCGVCSYKKR RMTKAQCCKC KKAICGEHNI DVCQDCI.
```

In certain embodiments, the piggyBac or piggyBac-like transposon is isolated or derived from Helicoverpa armigera. In certain embodiments, the piggyBac or piggyBac-like transposon comprises a sequence of:

```
(SEQ ID NO: 14570)
    1 ttaaccctag aagcccaatc tacgtaaatt tgacgtatac cgcggcgaaa tatctctgtc
  61 tctttcatgt ttaccgtcgg atcgccgcta acttctgaac caactcagta gccattggga
121 cctcgcagga cacagttgcg tcatctcggt aagtgccgcc attttgttgt actctctatt
181 acaacacacg tcacgtcacg tcgttgcacg tcattttgac gtataattgg gctttgtgta
241 acttttgaat ttgtttcaaa ttttttatgt ttgtgattta tttgagttaa tcgtattgtt
301 tcgttacatt tttcatataa taataatatt ttcaggttga gtacaaa.
```

In certain embodiments, the piggyBac or piggyBac-like transposon comprises a sequence of:

```
(SEQ ID NO: 14528)
    1 agactgtttt tttctaagag acttctaaaa tattattacg agttgattta attttatgaa
  61 aacatttaaa actagttgat tttttttata attacataat tttaagaaaa agtgttagag
121 gcttgatttt tttgttgatt ttttctaaga tttgattaaa gtgccataat agtattaata
181 aagagtattt tttaacttaa aatgtatttt atttattaat taaaacttca attatgataa
241 ctcatgcaaa aatatagttc attaacagaa aaaaatagga aaactttgaa gttttgtttt
301 tacacgtcat ttttacgtat gattgggctt tatagctagt taaatatgat tgggcttcta
361 gggttaa.
```

In certain embodiments of the methods of the disclosure, the transposase enzyme is a piggyBac or piggyBac-like transposase enzyme. In certain embodiments, the piggyBac or piggyBac-like transposase enzyme is isolated or derived from Pectinophora gossypiella. The piggyBac or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14530)
    1 MDLRKQDEKI RQWLEQDIEE DSKGESDNSS SETEDIVEME VHKNTSSESE VSSESDYEPV
  61 CPSKRQRTQI IESEESDNSE SIRPSRRQTS RVIDSDETDE DVMSSTPQNI PRNPNVIQPS
121 SRFLYGKNKH KWSSAAKPSS VRTSRRNIIH FIPGPKERAR EVSEPIDIFS LFISEDMLQQ
181 VVTFTNAEML IRKNKYKTET FTVSPTNLEE IRALLGLLFN AAAMKSNHLP TRMLFNTHRS
241 GTIFKACMSA ERLNFLIKCL RFDDKLTRNV RQRDDRFAPI RDLWQALISN FQKWYTPGSY
301 ITVDEQLVGF RGRCSFRMYI PNKPNKYGIK LVMAADVNSK YIVNAIPYLG KGTDPQNQPL
361 ATFFIKEITS TLHGTNRNIT MDNWFTSVPL ANELLMAPYN LTLVGTLRSN KREIPEKLKN
421 SKSRAIGTSM FCYDGDKTLV SYKAKSNKVV FILSTIHDQP DINQETGKPE MIHFYNSTKG
481 AVDTVDQMCS SISTNRKTQR WPLCVFYNML NLSIINAYVV YVYNNVRNNK KPMSRRDFVI
541 KLGDQLMEPW LRQRLQTVTL RRDIKVMIQD ILGESSDLEA PVPSVSNVRK IYYLCPSKAR
601 RMTKHRCIKC KQAICGPHNI DICSRCIE.
```

In certain embodiments, the piggyBac or piggyBac-like transposon is isolated or derived from Pectinophora gossypiella. In certain embodiments, the piggyBac or piggyBac-like transposon comprises a sequence of:

```
(SEQ ID NO: 14532)
    1 ttaaccctag ataactaaac attcgtccgc tcgacgacgc gctatgccgc gaaattgaag
  61 tttacctatt attccgcgtc ccccgccccc gccgcttttt ctagcttcct gatttgcaaa
121 atagtgcatc gcgtgacacg ctcgaggtca cacgacaatt aggtcgaaag ttacaggaat
181 ttcgtcgtcc gctcgacgaa agtttagtaa ttacgtaagt ttggcaaagg taagtgaatg
241 aagtattttt ttataattat tttttaattc tttatagtga taacgtaagg tttatttaaa
301 tttattactt ttatagttat ttagccaatt gttataaatt ccttgttatt gctgaaaaat
361 ttgcctgttt tagtcaaaat ttattaactt ttcgatcgtt ttttag.
```

In certain embodiments, the piggyBac or piggyBac-like transposon comprises a sequence of:

```
(SEQ ID NO: 14571)
    1 tttcactaag taattttgtt cctatttagt agataagtaa cacataatta ttgtgatatt
  61 caaaacttaa gaggtttaat aaataataat aaaaaaaaaa tggtttttat ttcgtagtct
121 gctcgacgaa tgtttagtta ttacgtaacc gtgaatatag tttagtagtc tagggttaa.
```

In certain embodiments of the methods of the disclosure, the transposase enzyme is a piggyBac or piggyBac-like transposase enzyme. In certain embodiments, the piggyBac or piggyBac-like transposase enzyme is isolated or derived from Ctenoplusia agnata. The piggyBac or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14534)
    1 MASRQHLYQD EIAAILENED DYSPHDTDSE MEDCVTQDDV RSDVEDEMVD NIGNGTSPAS
  61 RHEDPETPDP SSEASNLEVT LSSHRIIILP QRSIREKNNH IWSTTKGQSS GRTAAINIVR
121 TNRGPTRMCR NIVDPLLCFQ LFIKEEIVEE IVKWTNVEMV QKRVNLKDIS ASYRDTNEME
181 IWAIISMLTL SAVMKDNHLS TDELFNVSYG TRYVSVMSRE RFEFLLRLLR MGDKLLRPNL
241 RQEDAFTPVR KIWEIFINQC RLNYVPGTNL TVDEQLLGFR GRCPFRMYIP NKPDKYGIKF
301 PMVCDAATKY MVDAIPYLGK STKTQGLPLG EFYVKELTQT VHGTNRNVTC DNWFTSVPLA
361 KSLLNSPYNL TLVGTIRSNK REIPEEVKNS RSRQVGSSMF CFDGPLTLVS YKPKPSKMVF
421 LLSSCNEDAV VNQSNGKPDM ILFYNQTKGG VDSFDQMCSS MSTNRKTNRW PMAVFYGMLN
481 MAFVNSYIIY CHNMLAKKEK PLSRKDFMKK LSTDLTTPSM QKRLEAPTLK RSLRDNITNV
541 LKIVPQAAID TSFDEPEPKK RRYCGFCSYK KKRMTKTQCF KCKKPVCGEH NIDVCQDCI.
```

In certain embodiments, the piggyBac or piggyBac-like transposon is isolated or derived from Ctenoplusia agnata. In certain embodiments, the piggyBac or piggyBac-like transposon comprises a sequence of:

```
(SEQ ID NO: 14535)
    1 ttaaccctag aagcccaatc tacgtcattc tgacgtgtat gtcgccgaaa atactctgtc
  61 tctttctcct gcacgatcgg attgccgcga acgctcgatt caacccagtt ggcgccgaga
121 tctattggag gactgcggcg ttgattcggt aagtcccgcc attttgtcat agtaacagta
181 ttgcacgtca gcttgacgta tatttgggct ttgtgttatt tttgtaaatt ttcaacgtta
241 gtttattatt gcatcttttt gttacattac tggtttattt gcatgtatta ctcaaatatt
301 atttttattt tagcgtagaa aataca.
```

In certain embodiments, the piggyBac or piggyBac-like transposon comprises a sequence of:

```
(SEQ ID NO: 14536)
    1 agactgtttt ttttgtattt gcattatata ttatattcta aagttgattt aattctaaga
  61 aaaacattaa aataagtttc tttttgtaaa atttaattaa ttataagaaa aagtttaagt
121 tgatctcatt ttttataaaa atttgcaatg tttccaaagt tattattgta aaagaataaa
181 taaaagtaaa ctgagtttta attgatgttt tattatatca ttatactata tattacttaa
241 ataaaacaat aactgaatgt atttctaaaa ggaatcacta gaaaatatag tgatcaaaaa
301 tttacacgtc atttttgcgt atgattgggc tttataggtt ctaaaaatat gattgggcct
361 ctagggttaa.
```

In certain embodiments, the piggyBac or piggyBac-like transposon comprises an ITR sequence of CCCTAGAAGCCCAATC (SEQ ID NO: 14564).

In certain embodiments of the methods of the disclosure, the transposase enzyme is a piggyBac or piggyBac-like transposase enzyme. In certain embodiments, the piggyBac or piggyBac-like transposase enzyme is isolated or derived from Agrotis ipsilon. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14537)
    1 MESRQRLNQD EIATILENDD DYSPLDSDSE AEDRVVEDDV WSDNEDAMID YVEDTSRQED
  61 PDNNIASQES ANLEVTSLTS HRIISLPQRS ICGKNNHVWS TTKGRTTGRT SAINIIRTNR
121 GPTRMCRNIV DPLLCFQLFI TDEIIHEIVK WTNVEMIVKR QNLIDISASY RDTNTMEMWA
181 LVGILTLTAV MKDNHLSTDE LFDATFSGTR YVSVMSRERF EFLIRCMRMD DKTLRPTLRS
241 DDAFIPVRKL WEIFINQCRL NYVPGGNLTV DEQLLGFRGR CPFRMYIPNK PDKYGIRFPM
301 MCDAATKYMI DAIPYLGKST KTNGLPLGEF YVKELTKTVH GTNRNVTCDN WFTSIPLAKN
361 MLQAPYNLTI VGTIRSNKRE IPEEIKNSRS RPVGSSMFCF DGPLTLVSYK PKPSRMVFLL
421 SSCDENAVIN ESNGKPDMIL FYNQTKGGVD SFDQMCKSMS ANRKTNRWPM AVFYGMLNMA
481 FVNSYIIYCH NKINKQKKPI NRKEFMKNLS TDLTTPWMQE RLKAPTLKRT LRDNITNVLK
541 NVVPPSPANN SEEPGPKKRS YCGFCSYKKR RMTKTQFYKC KKAICGEHNI DVCQDCV.
```

In certain embodiments, the piggyBac or piggyBac-like transposon is isolated or derived from Agrotis ipsilon. In certain embodiments, the piggyBac or piggyBac-like transposon comprises a sequence of:

```
(SEQ ID NO: 14538)
    1 ttaaccctag aagcccaatc tacgtaaatt tgacgtatac cgcggcgaaa tatatctgtc
  61 tctttcacgt ttaccgtcgg attcccgcta acttcggaac caactcagta gccattgaga
121 actcccagga cacagttgcg tcatctcggt aagtgccgcc attttgttgt aatagacagg
181 ttgcacgtca ttttgacgta taattgggct ttgtgtaact tttgaaatta tttataattt
241 ttattgatgt gatttatttg agttaatcgt attgtttcgt tacatttttc atatgatatt
301 aatattttca gattgaatat aaa.
```

In certain embodiments, the piggyBac or piggyBac-like transposon comprises a sequence of:

```
(SEQ ID NO: 14539)
    1 agactgtttt ttttaaaagg cttataaagt attactattg cgtgatttaa ttttataaaa
  61 atatttaaaa ccagttgatt tttttaataa ttacctaatt ttaagaaaaa atgttagaag
121 cttgatattt ttgttgattt ttttctaaga tttgattaaa aggccataat tgtattaata
181 aagagtattt ttaacttcaa atttatttta tttattaatt aaaacttcaa ttatgataat
241 acatgcaaaa atatagttca tcaacagaaa aatataggaa aactctaata gttttatttt
301 tacacgtcat ttttacgtat gattgggctt tatagctagt caaatatgat tgggcttcta
361 gggttaa.
```

In certain embodiments of the methods of the disclosure, the transposase enzyme is a piggyBac or piggyBac-like transposase enzyme. In certain embodiments, the piggyBac or piggyBac-like transposase enzyme is isolated or derived from Megachile rotundata. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%. 70%. 75%. 80%. 85%. 90%. 95%. 99% or any percentage in between identical to:

```
(SEQ ID NO: 14540)
    1 MNGKDSLGEF YLDDLSDCLD CRSASSTDDE SDSSNIAIRK RCPIPLIYSD SEDEDMNNNV
  61 EDNNHFVKES NRYHYQIVEK YKITSKTKKW KDVTVTEMKK FLGLIILMGQ VKKDVLYDYW
121 STDPSIETPF FSKVMSRNRF LQIMQSWHFY NNNDISPNSH RLVKIQPVID YFKEKFNNVY
181 KSDQQLSLDE CLIPWRGRLS IKTYNPAKIT KYGILVRVLS EARTGYVSNF CVYAADGKKI
241 EETVLSVIGP YKNMWHHVYQ DNYYNSVNIA KIFLKNKLRV CGTIRKNRSL PQILQTVKLS
301 RGQHQFLRNG HTLLEVWNNG KRNVNMISTI HSAQMAESRN RSRTSDCPIQ KPISIIDYNK
361 YMKGVDRADQ YLSYYSIFRK TKKWTKRVVM FFINCALFNS FKVYTTLNGQ KITYKNFLHK
421 AALSLIEDCG TEEQGTDLPN SEPTTTRTTS RVDHPGRLEN FGKHKLVNIV TSGQCKKPLR
481 QCRVCASKKK LSRTGFACKY CNVPLHKGDC FERYHSLKKY.
```

In certain embodiments, the piggyBac or piggyBac-like transposon is isolated or derived from Megachile rotundata. In certain embodiments, the piggyBac or piggyBac-like transposon comprises a sequence of:

```
(SEQ ID NO: 14541)
    1 ttaaataatg cccactctag atgaacttaa cactttaccg accggccgtc gattattcga
  61 cgtttgctcc ccagcgctta ccgaccggcc atcgattatt cgacgtttgc ttcccagcgc
121 ttaccgaccg gtcatcgact tttgatcttt ccgttagatt tggttaggtc agattgacaa
181 gtagcaagca tttcgcattc tttattcaaa taatcggtgc ttttttctaa gctttagccc
241 ttagaa.
```

In certain embodiments, the piggyBac or piggyBac-like transposon comprises a sequence of:

```
(SEQ ID NO: 14542)
    1 acaacttctt ttttcaacaa atattgttat atggattatt tatttattta tttatttatg
  61 gtatatttta tgtttattta tttatggtta ttatggtata ttttatgtaa ataataaact
121 gaaaacgatt gtaatagatg aaataaatat tgttttaaca ctaatataat taaagtaaaa
181 gattttaata aatttcgtta ccctacaata acacgaagcg tacaatttta ccagagttta
241 ttaa.
```

In certain embodiments of the methods of the disclosure, the transposase enzyme is a piggyBac or piggyBac-like transposase enzyme. In certain embodiments, the piggyBac or piggyBac-like transposase enzyme is isolated or derived from Bombus impatiens. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14543)
    1 MNEKNGIGEF YLDDLSDCPD SYSRSNSGDE SDGSDTIIRK RGSVLPPRYS DSEDDEINNV
  61 EDNANNVENN DDIWSTNDEA IILEPFEGSP GLKIMPSSAE SVTDNVNLFF GDDFFEHLVR
121 ESNRYHYQVM EKYKIPSKAK KWTDITVPEM KKFLGLIVLM GQIKKDVLYD YWSTDPSIET
181 PFFSQVMSRN RFVQIMQSWH FCNNDNIPHD SHRLAKIQPV IDYFRRKFND VYKPCQQLSL
241 DESIIPWRGR LSIKTYNPAK ITKYGILVRV LSEAVTGYVC NFDVYAADGK KLEDTAVIEP
301 YKNIWHQIYQ DNYYNSVKMA RILLKNKVRV CGTIRKNRGL PRSLKTIQLS RGQYEFRRNH
361 QILLEVWNNG RRNVNMISTI HSAQLMESRS KSKRSDVPIQ KPNSIIDYNK YMKGVDRADQ
421 YLAYYSIFRK TKKWTKRVVM FFINCALFNS FRVYTILNGK NITYKNFLHK VAVSWIEDGE
481 TNCTEQDDNL PNSEPTRRAP RLDHPGRLSN YGKHKLINIV TSGRSLKPQR QCRVCAVQKK
541 RSRTCFVCKF CNVPLHKGDC FERYHTLKKY.
```

In certain embodiments, the piggyBac or piggyBac-like transposon is isolated or derived from Bombus impatiens. In certain embodiments, the piggyBac or piggyBac-like transposon comprises a sequence of:

```
(SEQ ID NO: 14544)
    1 ttaatttttt aacattttac cgaccgatag ccgattaatc gggtttttgc cgctgacgct
  61 taccgaccga taacctatta atcggctttt tgtcgtcgaa gcttaccaac ctatagccta
121 cctatagtta atcggttgcc atggcgataa acaatctttc tcattatatg agcagtaatt
181 tgttatttag tactaaggta ccttgctcag ttgcgtcagt tgcgttgctt tgtaagctcc
241 cacagtttta taccaattcg aaaaacttac cgttcgcg.
```

In certain embodiments, the piggyBac or piggyBac-like transposon comprises a sequence of:

```
(SEQ ID NO: 14545)
    1 actatttcac atttgaacta aaaaccgttg taatagataa aataaatata atttagtatt
  61 aatattatgg aaacaaaaga ttttattcaa tttaattatc ctatagtaac aaaaagcggc
121 caattttatc tgagcatacg aaaagcacag atactcccgc ccgacagtct aaaccgaaac
181 agagccggcg ccagggagaa tctgcgcctg agcagccggt cggacgtgcg tttgctgttg
241 aaccgctagt ggtcagtaaa ccagaaccag tcagtaagcc agtaactgat cagttaacta
301 gattgtatag ttcaaattga acttaatcta gtttttaagc gtttgaatgt tgtctaactt
361 cgttatatat tatattcttt ttaa.
```

In certain embodiments of the methods of the disclosure, the transposase enzyme is a piggyBac or piggyBac-like transposase enzyme. In certain embodiments, the piggyBac or piggyBac-like transposase enzyme is isolated or derived from Mamestra brassicae. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14546)
    1 MFSFVPNKEQ TRTVLIFCFH LKTTAAESHR PLVEAFGEQV PTVKTCERWF QRFKSGDFDV
  61 DDKEHGKPPK RYEDAELQAL LDEDDAQTQK QLAEQLEVSQ QAVSNRLREG GKIQKVGRWV
121 PHELNERQRE RRKNTCEILL SRYKRKSFLH RIVTGEEKWI FFVNPKRKKS YVDPGQPATS
181 TARPNRFGKK TRLCVWWDQS GVIYYELLKP GETVNTARYQ QQLINLNRAL QRKRPEYQKR
241 QHRVIFLHDN APSHTARAVR DTLETLNWEV LPHAAYSPDL APSDYHLFAS MGHALAEQRF
301 DSYESVEEWL DEWFAAKDDE FYWRGIHKLP ERWDNCVASD GKYFE.
```

In certain embodiments, the piggyBac or piggyBac-like transposon is isolated or derived from Mamestra brassicae. In certain embodiments, the piggyBac or piggyBac-like transposon comprises a sequence of:

```
(SEQ ID NO: 14547)
    1 ttattgggtt gcccaaaaag taattgcgga tttttcatat acctgtcttt taaacgtaca
  61 tagggatcga actcagtaaa actttgacct tgtgaaataa caaacttgac tgtccaacca
121 ccatagtttg gcgcgaattg agcgtcataa ttgttttgac tttttgcagt caac.
```

In certain embodiments, the piggyBac or piggyBac-like transposon comprises a sequence of:

(SEQ ID NO: 14548)
1 atgatttttt ctttttaaac caattttaat tagttaattg atataaaaat ccgcaattac
61 tttttgggca acccaataa.

In certain embodiments of the methods of the disclosure, the transposase enzyme is a piggyBac or piggyBac-like transposase enzyme. In certain embodiments, the piggyBac or piggyBac-like transposase enzyme is isolated or derived from Mayetiola destructor. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14549)
    1 MENFENWRKR RHLREVLLGH FFAKKTAAES HRLLVEVYGE HALAKTQCFE WFQRFKSGDF
  61 DTEDKERPGQ PKKFEDEELE ALLDEDCCQT QEELAKSLGV TQQAISKRLK AAGYIQKQGN
121 WVPHELKPRD VERRFCMSEM LLQRHKKKSF LSRIITGDEK WIHYDNSKRK KSYVKRGGRA
181 KSTPKSNLHG AKVMLCIWWD QRGVLYYELL EPGQTITGDL YRTQLIRLKQ ALAEKRPEYA
241 KRHGAVIFHH DNARPHVALP VKNYLENSGW EVLPHPPYSP DLAPSDYHLF RSMQNDLAGK
301 RFTSEQGIRK WLDSFLAAKP AKFFEKGIHE LSERWEKVIA SDGQYFE.
```

In certain embodiments, the piggyBac or piggyBac-like transposon is isolated or derived from Mayetiola destructor. In certain embodiments, the piggyBac or piggyBac-like transposon comprises a sequence of:

```
(SEQ ID NO: 14550)
    1 taagacttcc aaaatttcca cccgaacttt accttccccg cgcattatgt ctctcttttc
  61 accctctgat ccctggtatt gttgtcgagc acgatttata ttgggtgtac aacttaaaaa
121 ccggaattgg acgctagatg tccacactaa cgaatagtgt aaaagcacaa atttcatata
181 tacgtcattt tgaaggtaca tttgacagct atcaaaatca gtcaataaaa ctattctatc
241 tgtgtgcatc atattttttt attaact.
```

In certain embodiments, the piggyBac or piggyBac-like transposon comprises a sequence of:

```
(SEQ ID NO: 14551)
    1 tgcattcatt cattttgtta tcgaaataaa gcattaattt tcactaaaaa attccggttt
  61 ttaagttgta cacccaatat catccttagt gacaattttc aaatggcttt cccattgagc
121 tgaaaccgtg gctctagtaa gaaaaacgcc caacccgtca tcatatgcct tttttttctc
181 aacatccg.
```

In certain embodiments of the methods of the disclosure, the transposase enzyme is a piggyBac or piggyBac-like transposase enzyme. In certain embodiments, the piggyBac or piggyBac-like transposase enzyme is isolated or derived from Apis mellifera. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14552)
    1 MENQKEHYRH ILLFYFRKGK NASQAHKKLC AVYGDEALKE RQCQNWFDKF RSGDFSLKDE
  61 KRSGRPVEVD DDLIKAIIDS DRHSTTREIA EKLHVSHTCI ENHLKQLGYV QKLDTWVPHE
121 LKEKHLTQRI NSCDLLKKRN ENDPFLKRLI TGDEKWVVYN NIKRKRSWSR PREPAQTTSK
181 AGIHRKKVLL SVWWDYKGIV YFELLPPNRT INSVVYIEQL TKLNNAVEEK RPELTNRKGV
241 VFHHDNARPH TSLVTRQKLL ELGWDVLPHP PYSPDLAPSD YFLFRSLQNS LNGKNFNNDD
301 DIKSYLIQFF ANKNQKFYER GIMMLPERWQ KVIDQNGQHI TE.
```

In certain embodiments, the piggyBac or piggyBac-like transposon is isolated or derived from Apis mellifera. In certain embodiments, the piggyBac or piggyBac-like transposon comprises a sequence of:

```
(SEQ ID NO: 14553)
    1 ttgggttggc aactaagtaa ttgcggattt cactcataga tggcttcagt tgaattttta
  61 ggtttgctgg cgtagtccaa atgtaaaaca cattttgtta tttgatagtt ggcaattcag
121 ctgtcaatca gtaaaaaaag ttttttgatc ggttgcgtag ttttcgtttg gcgttcgttg
181 aaaa.
```

In certain embodiments, the piggyBac or piggyBac-like transposon comprises a sequence of:

(SEQ ID NO: 14554)
1 agttatttag ttccatgaaa aaattgtctt tgattttcta aaaaaaatcc gcaattactt
61 agttgccaat ccaa.

In certain embodiments of the methods of the disclosure, the transposase enzyme is a piggyBac or piggyBac-like transposase enzyme. In certain embodiments, the piggyBac or piggyBac-like transposase enzyme is isolated or derived from Messor bouvieri. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14555)
    1 MSSFVPENVH LRHALLFLFH QKKRAAESHR LLVETYGEHA PTIRTCETWF RQFKCGDFNV
  61 QDKERPGRPK TFEDAELQEL LDEDSTQTQK QLAEKLNVSR VAICERLQAM GKIQKMGRWV
121 PHELNDRQME NRKIVSEMLL QRYERKSFLH RIVTGDEKWI YFENPKRKKS WLSPGEAGPS
181 TARPNRFGRK TMLCVWWDQI GVVYYELLKP GETVNTDRYR QQMINLNCAL IEKRPQYAQR
241 HDKVILQHDN APSHTAKPVK EMLKSLGWEV LSHPPYSPDL APSDYHLFAS MGHALAEQHF
301 ADFEEVKKWL DEWFSSKEKL FFWNGIHKLS ERWTKCIESN GQYFE.
```

In certain embodiments, the piggyBac or piggyBac-like transposon is isolated or derived from Messor bouvieri. In certain embodiments, the piggyBac or piggyBac-like transposon comprises a sequence of:

```
(SEQ ID NO: 14556)
    1 agtcagaaat gacacctcga tcgacgacta atcgacgtct aatcgacgtc gattttatgt
  61 caacatgtta ccaggtgtgt cggtaattcc tttccggttt ttccggcaga tgtcactagc
121 cataagtatg aaatgttatg atttgataca tatgtcattt tattctactg acattaacct
181 taaaactaca caagttacgt tccgccaaaa taacagcgtt atagatttat aattttttga
241 aa.
```

In certain embodiments, the piggyBac or piggyBac-like transposon comprises a sequence of:

```
(SEQ ID NO: 14557)
    1 ataaatttga actatccatt ctaagtaacg tgttttcttt aacgaaaaaa ccggaaaaga
  61 attaccgaca ctcctggtat gtcaacatgt tattttcgac attgaatcgc gtcgattcga
121 agtcgatcga ggtgtcattt ctgact.
```

In certain embodiments of the methods of the disclosure, the transposase enzyme is a piggyBac or piggyBac-like transposase enzyme. In certain embodiments, the piggyBac or piggyBac-like transposase enzyme is isolated or derived from Trichoplusia ni. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14558)
    1 MGSSLDDEHI LSALLQSDDE LVGEDSDSEV SDHVSEDDVQ SDTEEAFIDE VHEVQPTSSG
  61 SEILDEQNVI EQPGSSLASN RILTLPQRTI RGKNKHCWST SKSTRRSRVS ALNIVRSQRG
121 PTRMCRNIYD PLLCFKLFFT DEIISEIVKW TNAEISLKRR ESMTSATFRD TNEDEIYAFF
181 GILVMTAVRK DNHMSTDDLF DRSLSMVYVS VMSRDRFDFL IRCLRMDDKS IRPTLRENDV
241 FTPVRKIWDL FIHQCIQNYT PGAHLTIDEQ LLGFRGRCPF RVYIPNKPSK YGIKILMMCD
301 SGTKYMINGM PYLGRGTQTN GVPLGEYYVK ELSKPVHGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDTLD QMCSVMTCSR KTNRWPMALL YGMINIACIN
481 SFIIYSHNVS SKGEKVQSRK KFMRNLYMSL TSSFMRKRLE APTLKRYLRD NISNILPKEV
541 PGTSDDSTEE PVMKKRTYCT YCPSKIRRKA NASCKKCKKV ICREHNIDMC QSCF.
```

In certain embodiments, the piggyBac or piggyBac-like transposon is isolated or derived from Trichoplusia ni. In certain embodiments, the piggyBac or piggyBac-like transposon comprises a sequence of:

```
(SEQ ID NO: 14559)
    1 ttaaccctag aaagatagtc tgcgtaaaat tgacgcatgc attcttgaaa tattgctctc
  61 tctttctaaa tagcgcgaat ccgtcgctgt gcatttagga catctcagtc gccgcttgga
121 gctcccgtga ggcgtgcttg tcaatgcggt aagtgtcact gattttgaac tataacgacc
181 gcgtgagtca aaatgacgca tgattatctt ttacgtgact tttaagattt aactcatacg
241 ataattatat tgttatttca tgttctactt acgtgataac ttattatata tatattttct
301 tgttatagat atc.
```

In certain embodiments, the piggyBac or piggyBac-like transposon comprises a sequence of:

```
(SEQ ID NO: 14560)
    1 tttgttactt tatagaagaa attttgagtt tttgtttttt tttaataaat aaataaacat
  61 aaataaattg tttgttgaat ttattattag tatgtaagtg taaatataat aaaacttaat
121 atctattcaa attaataaat aaacctcgat atacagaccg ataaaacaca tgcgtcaatt
181 ttacgcatga ttatctttaa cgtacgtcac aatatgatta tctttctagg gttaa.
```

In certain embodiments, the piggyBac or piggyBac-like transposon comprises a sequence of:

```
(SEQ ID NO: 14561)
    1 ccctagaaag atagtctgcg taaaattgac gcatgcattc ttgaaatatt gctctctctt
  61 tctaaatagc gcgaatccgt cgctgtgcat ttaggacatc tcagtcgccg cttggagctc
121 ccgtgaggcg tgcttgtcaa tgcggtaagt gtcactgatt ttgaactata acgaccgcgt
181 gagtcaaaat gacgcatgat tatcttttac gtgactttta agatttaact catacgataa
241 ttatattgtt atttcatgtt ctacttacgt gataacttat tatatatata ttttcttgtt
301 atagatatc.
```

In certain embodiments, the piggyBac or piggyBac-like transposon comprises a sequence of:

```
(SEQ ID NO: 14562)
    1 tttgttactt tatagaagaa attttgagtt tttgtttttt tttaataaat aaataaacat
  61 aaataaattg tttgttgaat ttattattag tatgtaagtg taaatataat aaaacttaat
121 atctattcaa attaataaat aaacctcgat atacagaccg ataaaacaca tgcgtcaatt
181 ttacgcatga ttatctttaa cgtacgtcac aatatgatta tctttctagg g.
```

In certain embodiments, the piggyBac or piggyBac-like transposon comprises a sequence of:

```
(SEQ ID NO: 14609)
    1 tctaaatagc gcgaatccgt cgctgtgcat ttaggacatc tcagtcgccg cttggagctc
  61 ccgtgaggcg tgcttgtcaa tgcggtaagt gtcactgatt ttgaactata acgaccgcgt
121 gagtcaaaat gacgcatgat tatcttttac gtgactttta agatttaact catacgataa
181 ttatattgtt atttcatgtt ctacttacgt gataacttat tatatatata ttttcttgtt
241 atagatatc.
```

In certain embodiments, the piggyBac or piggyBac-like transposon comprises a sequence of:

```
(SEQ ID NO: 14610)
    1 tttgttactt tatagaagaa attttgagtt tttgtttttt tttaataaat aaataaacat
  61 aaataaattg tttgttgaat ttattattag tatgtaagtg taaatataat aaaacttaat
121 atctattcaa attaataaat aaacctcgat atacagaccg ataaaacaca tgcgtcaatt
181 ttacgcatga ttatctttaa cgtacgtcac aatatgatta tctttctagg g.
```

In certain embodiments, the piggyBac or piggyBac-like transposon comprises SEQ ID NO: 14561 and SEQ ID NO: 14562, and the piggyBac or piggyBac-like transposase comprises SEQ ID NO: 14558. In certain embodiments, the piggyBac or piggyBac-like transposon comprises SEQ ID NO: 14609 and SEQ ID NO: 14610, and the piggyBac or piggyBac-like transposase comprises SEQ ID NO: 14558.

In certain embodiments, the piggyBac or piggyBac-like transposon is isolated or derived from Aphis gossypii. In certain embodiments, the piggyBac or piggyBac-like transposon comprises an ITR sequence of CCTTCCAGCGGGCGCGC (SEQ ID NO: 14565).

In certain embodiments, the piggyBac or piggyBac-like transposon is isolated or derived from Chilo suppressalis. In certain embodiments, the piggyBac or piggyBac-like transposon comprises an ITR sequence of CCCAGATTAGCCT (SEQ ID NO: 14566).

In certain embodiments, the piggyBac or piggyBac-like transposon is isolated or derived from Heliothis virescens. In certain embodiments, the piggyBac or piggyBac-like transposon comprises an ITR sequence of CCCTTAATTACTCGCG (SEQ ID NO: 14567).

In certain embodiments, the piggyBac or piggyBac-like transposon is isolated or derived from Pectinophora gossypiella. In certain embodiments, the piggyBac or piggyBac-like transposon comprises an ITR sequence of CCCTAGATAACTAAAC (SEQ ID NO: 14568).

In certain embodiments, the piggyBac or piggyBac-like transposon is isolated or derived from Anopheles stephensi. In certain embodiments, the piggyBac or piggyBac-like transposon comprises an ITR sequence of CCCTAGAAAGATA (SEQ ID NO: 14569).

DNA transposons in the hAT family are widespread in plants and animals. A number of active hAT transposon systems have been identified and found to be functional, including but not limited to, the Hermes transposon, Ac transposon, hobo transposon, and the Tol2 transposon. The hAT family is composed of two families that have been classified as the AC subfamily and the Buster subfamily, based on the primary sequence of their transposases. Members of the hAT family belong to Class II transposable elements. Class II mobile elements use a cut and paste mechanism of transposition. hAT elements share similar transposases, short terminal inverted repeats, and an eight base-pairs duplication of genomic target.

Compositions and methods of the disclosure may comprise a TcBuster transposon and/or a TcBuster transposase.

Compositions and methods of the disclosure may comprise a TcBuster transposon and/or a hyperactive TcBuster transposase. A hyperactive TcBuster transposase demonstrates an increased excision and/or increased insertion frequency when compared to an excision and/or insertion frequency of a wild type TcBuster transposase. A hyperactive TcBuster transposase demonstrates an increased transposition frequency when compared to a transposition frequency of a wild type TcBuster transposase.

In some embodiments of the compositions and methods of the disclosure, a wild type TcBuster transposase comprises or consists of the amino acid sequence of:

```
(GenBank Accession No. ABF20545 and SEQ ID NO: 17900)
    1 MMLNWLKSGK LESQSQEQSS CYLENSNCLP PTLDSTDIIG EENKAGTTSR KKRKYDEDYL
  61 NFGFTWTGDK DEPNGLCVIC EQVVNNSSLN PAKLKRHLDT KHPTLKGKSE YFKRKCNELN
121 QKKHTFERYV RDDNKNLLKA SYLVSLRIAK QGEAYTIAEK LIKPCTKDLT TCVFGEKFAS
181 KVDLVPLSDT TISRRIEDMS YFCEAVLVNR LENAKCGFTL QMDESTDVAG LAILLVFVRY
241 IHESSFELDM LFCKALPTQT TGEEIFNLLN AYFEKHSIPW NLCYHICTDG AKAMVGVIKG
301 VIARIKKLVP DIKASHCCLH RHALAVKRIP NALHEVLNDA VKMINEIKSR PLNARVFALL
361 CDDLGSLHKN LLLHTEVRWL SRGKVLTRFW ELRDEIRIFF NEREFAGKIN DTSWLQNLAY
421 IADIFSYLNE VNLSLQGPNS TIFKVNSRIN SIKSKLKLWE ECITKNNTEC FANLNDFLET
481 SNTALDPNLK SNILEHLNGL KNTELEYFPP TCNNISWVEN PFNECGNVDT LPIKEREQLI
541 DIRTDTTLKS SFVPDGIGPF WIKLMDEFPE ISKRAVKELM PFVTTYLCEK SFSVYVATKT
601 KYRNRLDAED DMRLQLTTIH PDIDNLCNNK QAQKSH.
```

In some embodiments of the compositions and methods of the disclosure, a TcBuster Transposase comprises or consists of a sequence having at least 20%, 25%, 30%, 35%, 40%, 45%, 50%, 55%, 60%, 65%, 70%, 75%, 80%, 85%, 90%, 95%, 97%, 99% or any percentage identity in between to a wild type TcBuster transposase comprising or consisting of the amino acid sequence of:

```
(GenBank Accession No. ABF20545 and SEQ ID NO: 17900)
    1 MMLNWLKSGK LESQSQEQSS CYLENSNCLP PTLDSTDIIG EENKAGTTSR KKRKYDEDYL
  61 NFGFTWTGDK DEPNGLCVIC EQVVNNSSLN PAKLKRHLDT KHPTLKGKSE YFKRKCNELN
121 QKKHTFERYV RDDNKNLLKA SYLVSLRIAK QGEAYTIAEK LIKPCTKDLT TCVFGEKFAS
181 KVDLVPLSDT TISRRIEDMS YFCEAVLVNR LENAKCGFTL QMDESTDVAG LAILLVFVRY
241 IHESSFEEDM LFCKALPTQT TGEEIFNLLN AYFEKHSIPW NLCYHICTDG AKAMVGVIKG
301 VIARIKKLVP DIKASHCCLH RHALAVKRIP NALHEVLNDA VKMINFIKSR PLNARVFALL
361 CDDLGSLHKN LLLHTEVRWL SRGKVLTRFW ELRDEIRIFF NEREFAGKLN DTSWLQNLAY
421 IADIFSYLNE VNLSLQGPNS TIFKYNSRIN SIKSKLKLWE ECITKNNTEC FANLNDFLET
481 SNTALDPNLK SNILEHLNGL KNTFLEYFPP TCNNISWVEN PFNECGNVDT LPIKEREQLI
541 DIRTDTTLKS SFVPDGIGPF WIKLMDEFPE ISKRAVRELM PFVTTYLCEK SFSVYVKTKT
601 KYRNRLDAED DMRLQLTTIH PDIDNLCNNK QAQKSH.
```

In some embodiments of the compositions and methods of the disclosure, a wild type TcBuster transposase is encoded by a nucleic acid sequence comprising or consisting of:

```
(GenBank Accession No. DQ481197 and SEQ ID NO: 17901)
      1 atgatgttga attggctgaa aagtggaaag cttgaaagtc aatcacagga acagagttcc
    61 tgctaccttg agaactctaa ctgcctgcca ccaacactcg attctacaga tattatcggt
  121 gaagagaaca aagctggtac cacctctcgc aagaagcgga aatatgacga ggactatctg
  181 aacttcggtt ttacatggac tggcgacaag gatgagccca acggactttg tgtgatttgc
  241 gagcaggtag tcaacaattc ctcacttaac ccggccaaac tgaaacgcca tttggacaca
  301 aagcatccga cgcttaaagg caagagcgaa tacttcaaaa gaaaatgtaa cgagctcaat
  361 caaaagaagc atacttttga gcgatacgta agggacgata acaagaacct cctgaaagct
  421 tattatctcg tcagtttgag aatagctaaa cagggcgagg catataccat agcggagaag
  481 ttgatcaagc cttgcaccaa ggatctgaca acttgcgtat ttggagaaaa attcgcgagc
  541 aaagttgatc tcatccccct gtccgacaca actatttcaa ggcaaatcga agacatgaat
  501 tacttctgtg aagccgtgct agtgaacagg ttgaaaaatg ctaaatgtgg atttacgctg
  661 cagatggacg agtcaacaga tgttgccgat cttgcaatcc tgcttgtatt tgttaggtac
  721 atacatgaaa gctcttttga ggaggatatg ttgttctgca aagcacttcc cactcagacg
  781 acaggggagg agattttcaa tcttctcaat gcctatttcg aaaagcactc catcccatgg
  841 aatctgtgtt accacatttg cacagacggt gccaaggcaa tggtaggagt tattaaagga
  901 gtcatagcga gaataaaaaa actcgtccct gatataaaag ctagccactg ttgcctgcat
  961 cgccacgctt tggctgtaaa gcgaataccg aatgcattgc acgaggtgct caatgacgct
1021 gttaaaatga tcaacttcat caagtctcgg ccgttgaatg cgcgcgtctt cgctttgctg
1081 tgtgacgatt tggagagcct gcataaaaat cttcttcttc ataccgaagt gaggtggctg
1141 tctagaagaa aggtgctgac ccgattttgg gaactaagag atgaaattag aattttcttc
1201 aacgaaaggg aatttgccgg gaaattgaac gacaccagtt gattgcaaaa tttggcatat
1261 atagctgaca tattcagtta tctgaatgaa gttaatcttt ccctgcaagg gccgaatagc
1321 acaatcttca aggtaaatag ccgcattaac agtattaaat caaagttgaa gttgtgggaa
1381 gagtgtataa cgaaaaataa cactgagtgt tttgcgaacc tcaacgdttt tttggaaact
1441 tcaaacactg cgttggatcc aaacctgaag tctaatattt tggaacatct caacggtctt
1501 aagaacacct ttctggagta ttttccacct acgtgtaata atatctcctg ggtggagaat
1561 cctttcaatg aatgcggtaa cgtcgataca ctcccaataa aagagaggga acaattgatt
1621 gacatacgga ctgatacgac attgaaatct tcattcgtgc ctgatggtat aggaccattc
1681 tggatcaaac tgatggacga atttccagaa attagcaaac gagctgtcaa agagctcatg
1741 ccatttgtaa ccacttacct ctgtgagaaa tcattttccg tctatgtagc cacaaaaaca
1801 aaatatcgaa atagacttga tgctgaagac gatatacgac tccaacttac tactatccat
1861 ccagacattg acaacctttg taacaacaag caggctcaga aatcccactg a.
```

In some embodiments of the compositions and methods of the disclosure, a TcBuster Transposase comprises or consists of a sequence having at least 20%, 25%, 30%, 35%, 40%, 45%, 50%, 55%, 60%, 65%, 70%, 75%, 80%, 85%, 90%, 95%, 97%, 99% or any percentage identity in between to a wild type TcBuster transposase encoded by a nucleic acid sequence comprising or consisting of:

```
(GenBank Accession No. DQ481197 and SEQ ID NO: 17901)
      1 atgatgttga attggctgaa aagtggaaag cttgaaagtc aatcacagga acagagttcc
    61 tgctaccttg agaactctaa ctgcctgcca ccaacgctcg attctacaga tattatcggt
  121 gaagagaaca aagctggtac cacctctcgc aagaagcgga aatatgacga ggactatctg
  181 aacttcggtt ttacatggac tggcgacaag gatgagccca acggactttg tgtgatttgc
  241 gagcaggtag tcaacaattc ctcacttaac ccggccaaac tgaaacgcca tttggacaca
  301 aagcatccga cgcttaaagg caagagcgaa tacttcaaaa gaaaatgtaa cgagctcaat
  361 caaaagaagc atacttttga gcgatacgta agggacgata acaagaacct cctgaaagct
  421 tcttatctcg tcagtttgag aatagctaaa cagggcgagg catataccat agcggagaag
  481 ttgatcaagc cttgcaccaa ggatctgaca acttgcgtat ttggagaaaa attcgcgagc
  541 aaagttgatc tcgtccccct gtccgacacg actatttcaa ggcgaatcga agacatgagt
  501 tacttctgtg aagccgtgct ggtgaacagg ttgaaaaatg ctaaatgtgg gtttacgctg
  661 cagatggacg agtcaacaga tgttgccggt cttgcaatcc tgcttgtgtt tgttaggtac
  721 atacatgaaa gctcttttga ggaggatatg ttgttctgca aagcacttcc cactcagacg
  781 acaggggagg agattttcaa tcttctcaat gcctatttcg aaaagcactc catcccatgg
  841 aatctgtgtt accacatttg cacagacggt gccaaggcaa tggtaggagt tattaaagga
  901 gtcatagcga gaataaaaaa actcgtccct gatataaaag ctaaccactg ttgcctgcat
  961 cgccacactt tggctgtaaa acgaataccg aatgcattgc acgaggtgct caatgacgct
1021 gttaaaatga tcaacttcat caagtctcgg ccgttgaatg cgcgcgtctt cgctttgctg
1081 tgtgacgatt tggggagcct gcataaaaat cttcttcttc ataccgaagt gaggtggctg
1141 tctagaggaa aggtgctgac ccgattttgg gaactgagag atgaaattag aattttcttc
1201 aacgaaaggg aatttgccgg gaaattgaac gacaccagtt ggttgcaaaa tttggcatat
1251 atagctgaca tattcagtta tctgaatgaa gttaatcttt ccctgcaagg gccgaatagc
1321 acaatcttca aggtaaatag ccgcattaac agtattaaat caaagttgaa gttgtgggaa
1381 gagtgtataa cgaaaaataa cactgagtgt tttgcgaacc tcaacgattt tttggaaact
1441 tcaaacactg cgttggatcc aaacctgaaa tctaatattt tgaaacatct caacggtctt
1501 aagaacacct ttctggagta ttttccacct acgtgtaata atatctcctg aatggagaat
1561 cctttcaatg aatacggtaa cgtcgataca ctcccaataa aaaagaggga acaattgatt
1621 gacatacgga ctgatacgac attgaaatct tcattcgtgc ctgatggtat aggaccattc
1681 tggatcaaac tgatggacga atttccagaa attagcaaac gagctgtcaa agagctcatg
1741 ccatttgtaa ccacttacct ctgtgagaaa tcattttccg tctatgtagc cacaaaaaca
1801 aaatatcgaa atagacttga tgctgaagac gatatgcgac tccaacttac tactatccat
1861 ccagacattg acaacctttg taacaacaag caggctcaga aatcccactg a.
```

In some embodiments of the compositions and methods of the disclosure, a TcBuster Transposase comprises or consists of a naturally occurring amino acid sequence.

In some embodiments of the compositions and methods of the disclosure, a TcBuster Transposase comprises or consists of a non-naturally occurring amino acid sequence.

In some embodiments of the compositions and methods of the disclosure, a TcBuster Transposase is encoded by a sequence comprising or consisting of a naturally occurring nucleic acid sequence.

In some embodiments of the compositions and methods of the disclosure, a TcBuster Transposase is encoded by a sequence comprising or consisting of a non-naturally occurring nucleic acid sequence.

In some embodiments of the compositions and methods of the disclosure, a mutant TcBuster Transposase comprises one or more sequence variations when compared to a wild type TcBuster Transposase. In some embodiments, the wild type TcBuster Transposase comprises or consists of the amino acid sequence of SEQ ID NO: 17900. In some embodiments, the wild type TcBuster Transposase is encoded by a sequence comprising or consisting of the nucleic acid sequence of SEQ ID NO: 17901. In some embodiments, the one or more sequence variations comprises one or more of a substitution, inversion, insertion, deletion, transposition, and frameshift. In some embodiments, the one or more sequence variations comprises a modified, synthetic, artificial or non-naturally occurring amino acid. In some embodiments, the one or more sequence variations comprises a modified, synthetic, artificial or non-naturally occurring nucleic acid.

In some embodiments of the compositions and methods of the disclosure, a mutant TcBuster Transposase comprises one or more sequence variations when compared to a wild type TcBuster Transposase. In some embodiments, the one or more sequence variations comprises an amino acid substitution in one or more of a DNA Binding and Oligomerization domain, an insertion domain and a Zn-BED domain.

In some embodiments of the compositions and methods of the disclosure, a mutant TcBuster Transposase comprises one or more sequence variations when compared to a wild type TcBuster Transposase. In some embodiments, the one or more sequence variations comprises an amino acid substitution that increases a net charge a neutral pH when compared to a wild type TcBuster Transposase. In some embodiments, the wild type TcBuster Transposase comprises or consists of the amino acid sequence of SEQ ID NO: 17900. In some embodiments, the wild type TcBuster Transposase is encoded by a sequence comprising or consisting of the nucleic acid sequence of SEQ ID NO: 17901. In some embodiments, the one or more sequence variations comprises an amino acid substitution of the aspartic acid (D) at position 223 (D223), the aspartic acid (D) at position 289 (D289) and the aspartic acid (E) at position 589 (E289) of SEQ ID NO: 17900. In some embodiments, the one or more sequence variations comprises an amino acid substitution within 5, 10, 15, 20, 25, 30, 35, 40, 45, 50, 55, 60, 65, 70, 75, 80, 85, 90, 95, 100 or any number of amino acids in between of position 223, 289 and/or 289 of SEQ ID NO: 17900. In some embodiments, the one or more sequence variations comprises an amino acid substitution within 70 amino acids of position 223, 289 and/or 289 of SEQ ID NO: 17900. In some embodiments, the one or more sequence variations comprises an amino acid substitution within 80 amino acids of position 223, 289 and/or 289 of SEQ ID NO: 17900. In some embodiments, the one or more sequence variations comprises an amino acid substitution of an aspartic acid (D) or a aspartic acid (E) to a neutral amino acid, a lysine (L) or an arginine (R) (e.g. D223L, D223R, D289L, D289R, E289L, E289R of SEQ ID NO: 17900).

In some embodiments of the compositions and methods of the disclosure, a mutant TcBuster Transposase comprises one or more sequence variations when compared to a wild type TcBuster Transposase. In some embodiments, the one or more sequence variations comprises one or more of Q82E, N85S, D99A, D132A, Q151S, Q151A, E153K, E153R, A154P, Y155H, E159A, T171K, T171R, K177E, D183K, D183R, D189A, T191E, S193K, S193R, Y201A, F202D, F202K, C2031, C203V, Q221T, M222L, I223Q, E224G, S225W, D227A, R239H, E243A, E247K, P257K, P257R, Q258T, E263A, E263K, E263R, E274K, E274R, S278K, N281E, L282K, L282R, K292P, V297K, K2995, A303T, H322E, A332S, A358E, A358K, A358S, D376A, V377T, L380N, I398D, I398S, I398K, F400L, V431L, S447E, N450K, N450R, I452F, E469K, K469K, P510D, P510N, E517R, R536S, V553S, P554T, P559D, P559S, P559K, K573E, E578L, K590T, Y595L, V596A, T598I, K599A, Q615A, T618K, T618K, T618R, D622K and D622R of SEQ ID NO: 17900. In some embodiments, the one or more sequence variations comprises an amino acid substitution within 5, 10, 15, 20, 25, 30, 35, 40, 45, 50, 55, 60, 65, 70, 75, 80, 85, 90, 95, 100 or any number of amino acids in between of position 154, 155, 159, 171, 177, 183, 189, 191, 193, 201, 202, 203, 221, 223, 224, 225, 227, 239, 243, 247, 257, 258, 263, 274, 278, 281, 282, 292, 297, 299, 303, 322, 332, 358, 376, 377, 380, 398, 400, 431, 447, 450, 452, 469, 510, 517, 536, 553, 554, 559, 573, 578, 590, 595, 596, 598, 599, 615, 618, and 622 of SEQ ID NO: 17900.

In some embodiments of the compositions and methods of the disclosure, a mutant TcBuster Transposase comprises one or more sequence variations when compared to a wild type TcBuster Transposase. In some embodiments, the one or more sequence variations comprises one or more of E247K, V297K, A358K, S278K, E247R, E274R, V297R, A358R, S278R, T171R, D183R, S193R, P257K, E263R, L282K, T618K, D622R, E153K, N450K, T171K, D183K, S193K, P257R, E263K, L282R, T618R, D622K, E153R and N450R of SEQ ID NO: 17900. In some embodiments, the one or more sequence variations comprises an amino acid substitution within 5, 10, 15, 20, 25, 30, 35, 40, 45, 50, 55, 60, 65, 70, 75, 80, 85, 90, 95, 100 or any number of amino acids in between of position 153, 171, 183, 193, 247, 257, 263, 274, 278, 282, 297, 358, 450, 618, 622 of SEQ ID NO: 17900.

In some embodiments of the compositions and methods of the disclosure, a mutant TcBuster Transposase comprises one or more sequence variations when compared to a wild type TcBuster Transposase. In some embodiments, the one or more sequence variations comprises one or more of V377T/E469K, V377T/E469K/R536S, A332S, V553S/P554T, E517R, K299S, Q615A/T618K, S278K, A303I, P510D, P510N, N281S, N281E, K590T, Q258T, E247K, S447E, N85S, V297K, A358K, I452F, V377T/E469K/D189A, K573E/E578L, I452FN377T/E469K/D189A, A358KN377T/E469K/D189A, K573E/E578L/V377T/E469K/D189A, T171R, D183R, S193R, P257K, E263R, L282K, T618K, D622R, E153K, N450K, T171K, D183K, S193K, P257R, E263K, L282R, T618R, D622K, E153R, N450R, E247K/E274KN297K/A358K of SEQ ID NO: 17900. In some embodiments, the one or more sequence variations comprises an amino acid substitution within 5, 10, 15, 20, 25, 30, 35, 40, 45, 50, 55, 60, 65, 70, 75, 80, 85, 90, 95, 100 or any number of amino acids in between of position 85, 153, 171, 189, 193, 247, 257, 258, 263, 274, 278, 281, 282, 297, 299, 303, 332, 358, 377, 450, 469, 447, 452, 469, 510, 517, 536, 553, 554, 573, 578, 590, 615, 618, 622 of SEQ ID NO: 17900.

In some embodiments of the compositions and methods of the disclosure, a mutant TcBuster Transposase comprises one or more sequence variations when compared to a wild type TcBuster Transposase. In some embodiments, the one or more sequence variations comprises one or more of V377T/E469K, V377T/E469K/R536S, V553S/P554T, Q615A/T618K, S278K, A303T, P510D, P510N, N281S, N281E, K590T, Q258T, E247K, S447E, N85S, V297K, A358K, I452F, V377T/E469K/D189A and K573E/E578L. In some embodiments, the one or more sequence variations comprises an amino acid substitution within 5, 10, 15, 20, 25, 30, 35, 40, 45, 50, 55, 60, 65, 70, 75, 80, 85, 90, 95, 100 or any number of amino acids in between of position 85, 189, 247, 258, 278, 281, 297, 303, 358, 377, 447, 452, 469, 510, 536, 553, 554, 573, 578, 590, 615, 618 of SEQ ID NO: 17900.

In some embodiments of the compositions and methods of the disclosure, a mutant TcBuster Transposase comprises one or more sequence variations when compared to a wild type TcBuster Transposase. In some embodiments, the one or more sequence variations comprises one or more of Q151S, Q151A, A154P, Q615A, V553S, Y155H, Y201A, F202D, F202K, C2031, C203V, F400L, I398D, I398S, I398K, V431L, P559D, P559S, P559K, M222L of SEQ ID NO: 17900. In some embodiments, the one or more sequence variations comprises an amino acid substitution within 5, 10, 15, 20, 25, 30, 35, 40, 45, 50, 55, 60, 65, 70, 75, 80, 85, 90, 95, 100 or any number of amino acids in between of position 151, 154, 615, 553, 155, 201, 202, 203, 400, 398, 431, 559, 222 of SEQ ID NO: 17900.

In some embodiments of the compositions and methods of the disclosure, a mutant TcBuster Transposase comprises one or more sequence variations when compared to a wild type TcBuster Transposase. In some embodiments, the one or more sequence variations comprises one or more of V377T, E469K, and D189A, when numbered in accordance with SEQ ID NO: 17900.

In some embodiments of the compositions and methods of the disclosure, a mutant TcBuster Transposase comprises one or more sequence variations when compared to a wild type TcBuster Transposase. In some embodiments, the one or more sequence variations comprises one or more of K573E and E578L, when numbered in accordance with SEQ ID NO: 17900.

In some embodiments, the mutant TcBuster transposase comprises amino acid substitution I452K, when numbered in accordance with SEQ ID NO: 17900.

In some embodiments of the compositions and methods of the disclosure, a mutant TcBuster Transposase comprises one or more sequence variations when compared to a wild type TcBuster Transposase. In some embodiments, the one or more sequence variations comprises one or more of A358K, when numbered in accordance with SEQ ID NO: 17900.

In some embodiments of the compositions and methods of the disclosure, a mutant TcBuster Transposase comprises one or more sequence variations when compared to a wild type TcBuster Transposase. In some embodiments, the one or more sequence variations comprises one or more of V297K, when numbered in accordance with SEQ ID NO: 17900.

In some embodiments of the compositions and methods of the disclosure, a mutant TcBuster Transposase comprises one or more sequence variations when compared to a wild type TcBuster Transposase. In some embodiments, the one or more sequence variations comprises one or more of N85S, when numbered in accordance with SEQ ID NO: 17900.

In some embodiments of the compositions and methods of the disclosure, a mutant TcBuster Transposase comprises one or more sequence variations when compared to a wild type TcBuster Transposase. In some embodiments, the one or more sequence variations comprises one or more of I452F, V377T, E469K, and D189A, when numbered in accordance with SEQ ID NO: 17900.

In some embodiments of the compositions and methods of the disclosure, a mutant TcBuster Transposase comprises one or more sequence variations when compared to a wild type TcBuster Transposase. In some embodiments, the one or more sequence variations comprises one or more of A358K, V377T, E469K, and D189A, when numbered in accordance with SEQ ID NO: 17900.

In some embodiments of the compositions and methods of the disclosure, a mutant TcBuster Transposase comprises one or more sequence variations when compared to a wild type TcBuster Transposase. In some embodiments, the one or more sequence variations comprises one or more of V377T, E469K, D189A, K573E and E578L, when numbered in accordance with SEQ ID NO: 17900.

In some embodiments of the compositions and methods of the disclosure, a TcBuster Transposase recognizes a 5′ inverted repeat comprising or consisting of the sequence of:

```
(SEQ NO: 17902)
    1 Cagtgttctt caacctttgc catccggcgg aaccctttgt cgagatattt ttttttatgg
  61 aacccttcat ttagtaatac acccagatga gattttaggg acagctgcgt tgacttgtta
121 cgaacaaggt gagcccgtgc tttggtctag ccaagaacat ggtaaagact atattcgcgg
181 cgttgtgaca atttaccgaa caactccgcg gccgggaagc cgatctcggc ttgaacgaat
241 tgttaggtgg cggtacttgg gtcgatatca aagtgcatca cttcttcccg tatgcccaac
301 tttgtataga gagccactgc gggatcgtca ccgtaatctg cttgcacgta gatcacataa
361 gcaccaagcg cgttggcctc atgcttgagg agattaatga gcgcggtggc aatgccctgc
421 ctccggtgct cgccggagac tgcgagatca tagatata
```

In some embodiments of the compositions and methods of the disclosure, a TcBuster Transposase recognizes a 3′ inverted repeat comprising or consisting of the sequence of:

```
(SEQ ID NO: 17903)
    1 gatatcaagc ttatcgatac cgtcgacctc gagatttctg aacaattcta ggttaggatc
  61 aaacaaaata caatttattt taaaactgta agttaactta cctttgcttg tctaaaccaa
121 aaacaacaac aaaactacga ccacaagtac agttacatat ttttgaaaat taaggttaag
181 tgcagtgtaa gtcaactatg cgaatggata acatgtttca acatgaaact ccgattgacg
241 catgtgcatt ctgaagagcg gcgcggccga cgtctctcga attgaagcaa tgactcgcgg
301 aaccccgaaa gcctttgggt ggaaccctag ggttccgcgg aacacaggtt gaagaacact
361 g
```

In some embodiments of the compositions and methods of the disclosure, a TcBuster Transposase recognizes a 5′ inverted repeat comprising or consisting of the sequence of SEQ ID NO: 17902 and a 3′ inverted repeat comprising or consisting of the sequence of SEQ ID NO: 17903.

In some embodiments of the compositions and methods of the disclosure, a TcBuster Transposase recognizes a 5′ inverted repeat comprising or consisting of the sequence of:

```
(SEQ ID NO: 17904)
    1 Cctgcaggag tgttcttcaa cctttgccat ccggcggaac cctttgtcga gatatttttt
  61 tttatggaac ccttcattta gtaatacacc cagatgagat tttagggaca gctgcgttga
121 cttgttacga acaaggtgag cccgtgcttt ggtaataaaa actctaaata agatttaaat
181 ttgcatttat ttaaacaaac tttaaacaaa aagataaata ttccaaataa aataatatat
241 aaaataaaaa ataaaaatta atgacttttt tgcgcttgct tattattgca caaattatca
301 atatcaagat ggatcgttgt ttttt.
```

In some embodiments of the compositions and methods of the disclosure, a TcBuster Transposase recognizes a 3′ inverted repeat comprising or consisting of the sequence of:

```
(SEQ ID NO: 17905)
    1 Gagccaattc agcatcatat ttctgaacga ttctaggtta ggatcaaaca aaatacaatt
  61 tattttaaaa ctgtaagtta acttaccttt gcttgtctaa acctaaaaca acaacaaaac
121 tacgaccaca agtacagtta catatttttg aaaattaagg ttaagtgcag tataagtcaa
181 ctatgcgaat ggataacatg tttcaacata aaactccgat tgacgcatgt gcattctgaa
241 gagcggcgcg gccgacgtct ctcgaattga agcaatgact cgcggaaccc cgaaagcctt
301 tgggtggaac cctagggttc cgcggaacac aggttgaaga acactg.
```

In some embodiments of the compositions and methods of the disclosure, a TcBuster Transposase recognizes a 5′ inverted repeat comprising or consisting of the sequence of SEQ ID NO: 17904 and a 3′ inverted repeat comprising or consisting of the sequence of SEQ ID NO: 17905.

In some embodiments of the compositions and methods of the disclosure, a TcBuster Transposase recognizes an inverted repeat comprising or consisting of a sequence having at least 5%, 10%, 15%, 20%, 25%, 30%, 35%, 40%, 45%, 50%, 55%, 60%, 65%, 70%, 75%, 80%, 85%, 90%, 95,% 97%, 99% or any percentage identify in between to one or more of SEQ ID NO: 17902, 17903, 17904 or 17905.

In some embodiments of the compositions and methods of the disclosure, a TcBuster Transposase recognizes an inverted repeat comprising or consisting of a sequence having at least In some embodiments of the compositions and methods of the disclosure, a TcBuster Transposase recognizes an inverted repeat comprising or consisting of a sequence having at least 5, 10, 15, 20, 25, 30, 35, 40, 45, 50, 55, 60, 65, 70, 75, 80, 85, 90, 95, 97, 99 or any number of contiguous nucleotides in between having between 90 and 100% identity to SEQ ID NO: 17902, 17903, 17904 or 17905 or any portion thereof.

In some embodiments of the compositions and methods of the disclosure, a TcBuster Transposase recognizes an inverted repeat comprising or consisting of a sequence having at least 5, 10, 15, 20, 25, 30, 35, 40, 45, 50, 55, 60, 65, 70, 75, 80, 85, 90, 95, 97, 99 or any number of discontinuous nucleotides in between having between 90 and 100% identity to SEQ ID NO: 17902, 17903, 17904 or 17905 or any portion thereof.

In some embodiments of the compositions and methods of the disclosure, a TcBuster transposon comprises a 3′ inverted repeat and a 5′ inverted repeat. In some embodiments of the compositions and methods of the disclosure, a TcBuster Transposase recognizes a TcBuster transposon comprising a 3′ inverted repeat and a 5′ inverted repeat comprising or consisting of a sequence having at least 5, 10, 15, 20, 25, 30, 35, 40, 45, 50, 55, 60, 65, 70, 75, 80 85, 90, 95, 97, 99 or any number of discontinuous nucleotides in between having between 90 and 100% identity to SEQ ID NO: 17902, 17903, 17904 or 17905 or any portion thereof

### Non-Transposition Based Methods of Genetic Modification

In some embodiments of the methods of the disclosure, a modified HSCor modified HSC descendent cell of the disclosure may be produced by introducing a transgene into an HSC or an HSC descendent cell of the disclosure. The introducing step may comprise delivery of a nucleic acid sequence and/or a genomic editing construct via a non-transposition delivery system.

In some embodiments of the methods of the disclosure, introducing a nucleic acid sequence and/or a genomic editing construct into an HSC or HSC descendent cell ex vivo, in vivo, in vitro or in situ comprises one or more of topical delivery, adsorption, absorption, electroporation, spin-fection, co-culture, transfection, mechanical delivery, sonic delivery, vibrational delivery, magnetofection or by nanoparticle-mediated delivery. In some embodiments of the methods of the disclosure, introducing a nucleic acid sequence and/or a genomic editing construct into an HSC or HSC descendent cell ex vivo, in vivo, in vitro or in situ comprises liposomal transfection, calcium phosphate transfection, fugene transfection, and dendrimer-mediated transfection. In some embodiments of the methods of the disclosure, introducing a nucleic acid sequence and/or a genomic editing construct into an HSC or HSC descendent cell ex vivo, in vivo, in vitro or in situ by mechanical transfection comprises cell squeezing, cell bombardment, or gene gun techniques. In some embodiments of the methods of the disclosure, introducing a nucleic acid sequence and/or a genomic editing construct into an HSC or HSC descendent cell ex vivo, in vivo, in vitro or in situ by nanoparticle-mediated transfection comprises liposomal delivery, delivery by micelles, and delivery by polymerosomes.

In some embodiments of the methods of the disclosure, introducing a nucleic acid sequence and/or a genomic editing construct into an HSC or HSC descendent cell ex vivo, in vivo, in vitro or in situ comprises a non-viral vector. In some embodiments, the non-viral vector comprises a nucleic acid. In some embodiments, the non-viral vector comprises plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). In some embodiments, the non-viral vector comprises a transposon of the disclosure.

In some embodiments of the methods of the disclosure, introducing a nucleic acid sequence and/or a genomic editing construct into an HSC or HSC descendent cell ex vivo, in vivo, in vitro or in situ comprises a viral vector. In some embodiments, the viral vector is a non-integrating non-chromosomal vector. Exemplary non-integrating non-chromosomal vectors include, but are not limited to, adeno-associated virus (AAV), adenovirus, and herpes viruses. In some embodiments, the viral vector is an integrating chromosomal vector. Integrating chromosomal vectors include, but are not limited to, adeno-associated vectors (AAV), Lentiviruses, and gamma-retroviruses.

In some embodiments of the methods of the disclosure, introducing a nucleic acid sequence and/or a genomic editing construct into an HSC or HSC descendent cell ex vivo, in vivo, in vitro or in situ comprises a combination of vectors. Exemplary, non-limiting vector combinations include: viral and non-viral vectors, a plurality of non-viral vectors, or a plurality of viral vectors. Exemplary but non-limiting vectors combinations include: a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

In some embodiments of the methods of the disclosure, genome modification comprising introducing a nucleic acid sequence and/or a genomic editing construct into an HSC or HSC descendent cell ex vivo, in vivo, in vitro or in situ stably integrates a nucleic acid sequence, transciently integrates a nucleic acid sequence, produces site-specific integration a nucleic acid sequence, or produces a biased integration of a nucleic acid sequence. In some embodiments, the nucleic acid sequence is a transgene.

In some embodiments of the methods of the disclosure, genome modification comprising introducing a nucleic acid sequence and/or a genomic editing construct into an HSC or HSC descendent cell ex vivo, in vivo, in vitro or in situ stably integrates a nucleic acid sequence. In some embodiments, the stable chromosomal integration can be a random integration, a site-specific integration, or a biased integration. In some embodiments, the site-specific integration can be non-assisted or assisted. In some embodiments, the assisted site-specific integration is co-delivered with a site-directed nuclease. In some embodiments, the site-directed nuclease comprises a transgene with 5′ and 3′ nucleotide sequence extensions that contain a percentage homology to upstream and downstream regions of the site of genomic integration. In some embodiments, the transgene with homologous nucleotide extensions enable genomic integration by homologous recombination, microhomology-mediated end joining, or nonhomologous end-joining. In some embodiments the site-specific integration occurs at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C-C motif) receptor 5 (CCRS) gene and the site of the human ortholog of the mouse Rosa26 locus.

In some embodiments, the site-specific transgene integration occurs at a site that disrupts expression of a target gene. In some embodiments, disruption of target gene expression occurs by site-specific integration at introns, exons, promoters, genetic elements, enhancers, suppressors, start codons, stop codons, and response elements. In some embodiments, exemplary target genes targeted by site-specific integration include but are not limited to TRAC, TRAB, PDI, any immunosuppressive gene, and genes involved in allo-rejection.

In some embodiments, the site-specific transgene integration occurs at a site that results in enhanced expression of a target gene. In some embodiments, enhancement of target gene expression occurs by site-specific integration at introns, exons, promoters, genetic elements, enhancers, suppressors, start codons, stop codons, and response elements.

In some embodiments of the methods of the disclosure, enzymes may be used to create strand breaks in the host genome to facilitate delivery or integration of the transgene. In some embodiments, enzymes create single-strand breaks. In some embodiments, enzymes create double-strand breaks. In some embodiments, examples of break-inducing enzymes include but are not limited to: transposases, integrases, endonucleases, CRISPR-Cas9, transcription activator-like effector nucleases (TALEN), zinc finger nucleases (ZFN), Cas-CLOVER™, and CPF1. In some embodiments, break-inducing enzymes can be delivered to the cell encoded in DNA, encoded in mRNA, as a protein, as a nucleoprotein complex with a guide RNA (gRNA).

In some embodiments of the methods of the disclosure, the site-specific transgene integration is controlled by a vector-mediated integration site bias. In some embodiments vector-mediated integration site bias is controlled by the chosen lentiviral vector. In some embodiments vector-mediated integration site bias is controlled by the chosen gamma-retroviral vector.

In some embodiments of the methods of the disclosure, the site-specific transgene integration site is a non-stable chromosomal insertion. In some embodiments, the integrated transgene may become silenced, removed, excised, or further modified.

In some embodiments of the methods of the disclosure, the genome modification is a non-stable integration of a transgene. In some embodiments, the non-stable integration can be a transient non-chromosomal integration, a semi-stable non chromosomal integration, a semi-persistent non-chromosomal insertion, or a non-stable chromosomal insertion. In some embodiments, the transient non-chromosomal insertion can be epi-chromosomal or cytoplasmic.

In some embodiments, the transient non-chromosomal insertion of a transgene does not integrate into a chromosome and the modified genetic material is not replicated during cell division.

In some embodiments of the methods of the disclosure, the genome modification is a semi-stable or persistent non-chromosomal integration of a transgene. In some embodiments, a DNA vector encodes a Scaffold/matrix attachment region (S-MAR) module that binds to nuclear matrix proteins for episomal retention of a non-viral vector allowing for autonomous replication in the nucleus of dividing cells.

In some embodiments of the methods of the disclosure, the genome modification is a non-stable chromosomal integration of a transgene. In some embodiments, the integrated transgene may become silenced, removed, excised, or further modified.

In some embodiments of the methods of the disclosure, the modification to the genome by transgene insertion can occur via host cell-directed double-strand breakage repair (homology-directed repair) by homologous recombination (HR), microhomology-mediated end joining (MMEJ), nonhomologous end joining (NHEJ), transposase enzyme-mediated modification, integrase enzyme-mediated modification, endonuclease enzyme-mediated modification, or recombinant enzyme-mediated modification. In some embodiments, the modification to the genome by transgene insertion can occur via CRISPR-Cas9, TALEN, ZFNs, Cas-CLOVER, and cpfl.

In gene editing systems that involve inserting new or existing nucleotides/nucleic acids, insertion tools (e.g. DNA template vectors, transposable elements (transposons or retrotransposons) must be delivered to the cell in addition to the cutting enzyme (e.g. a nuclease, recombinase, integrase or transposase). Examples of such insertion tools for a recombinase may include a DNA vector. Other gene editing systems require the delivery of an integrase along with an insertion vector, a transposase along with a transposon/retrotransposon, etc. In some embodiments, an example recombinase that may be used as a cutting enzyme is the CRE recombinase. In various embodiments, example integrases that may be used in insertion tools include viral based enzymes taken from any of a number of viruses including, but not limited to, AAV, gamma retrovirus, and lentivirus. Example transposons/retrotransposons that may be used in insertion tools include, but are not limited to, the piggyBac transposon, Sleeping Beauty transposon, and the L1 retrotransposon.

In certain embodiments of the methods of the disclosure, the transgene is delivered in vivo. In certain embodiments of the methods of the disclosure, in vivo transgene delivery can occur by: topical delivery, adsorption, absorption, electroporation, spin-fection, co-culture, transfection, mechanical delivery, sonic delivery, vibrational delivery, magnetofection or by nanoparticle-mediated delivery. In certain embodiments of the methods of the disclosure, in vivo transgene delivery by transfection can occur by liposomal transfection, calcium phosphate transfection, fugene transfection, and dendrimer-mediated transfection. In certain embodiments of the methods of the disclosure, in vivo mechanical transgene delivery can occur by cell squeezing, bombardment, and gene gun. In certain embodiments of the methods of the disclosure, in vivo nanoparticle-mediated transgene delivery can occur by liposomal delivery, delivery by micelles, and delivery by polymerosomes. In various embodiments, nucleases that may be used as cutting enzymes include, but are not limited to, Cas9, transcription activator-like effector nucleases (TALENs) and zinc finger nucleases.

In certain embodiments of the methods of the disclosure, non-viral vectors are used for transgene delivery. In certain embodiments, the non-viral vector is a nucleic acid. In certain embodiments, the nucleic acid non-viral vector is plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). In certain embodiments, the non-viral vector is a transposon. In certain embodiments, the transposon is piggyBac™.

In certain embodiments of the methods of the disclosure, transgene delivery can occur via viral vector. In certain embodiments, the viral vector is a non-integrating non-chromosomal vectors. Non-integrating non-chromosomal vectors can include adeno-associated virus (AAV), adenovirus, and herpes viruses. In certain embodiments, the viral vector is an integrating chromosomal vectors. Integrating chromosomal vectors can include adeno-associated vectors (AAV), Lentiviruses, and gamma-retroviruses.

In certain embodiments of the methods of the disclosure, transgene delivery can occur by a combination of vectors. Exemplary but non-limiting vector combinations can include: viral plus non-viral vectors, more than one non-viral vector, or more than one viral vector. Exemplary but non-limiting vectors combinations can include: DNA-derived plus RNA-derived vectors, RNA plus reverse transcriptase, a transposon and a transposase, a non-viral vectors plus an endonuclease, and a viral vector plus an endonuclease.

In certain embodiments of the methods of the disclosure, the genome modification can be a stable integration of a transgene, a transient integration of a transgene, a site-specific integration of a transgene, or a biased integration of a transgene.

In certain embodiments of the methods of the disclosure, the genome modification can be a stable chromosomal integration of a transgene. In certain embodiments, the stable chromosomal integration can be a random integration, a site-specific integration, or a biased integration. In certain embodiments, the site-specific integration can be non-assisted or assisted. In certain embodiments, the assisted site-specific integration is co-delivered with a site-directed nuclease. In certain embodiments, the site-directed nuclease comprises a transgene with 5′ and 3′ nucleotide sequence extensions that contain homology to upstream and downstream regions of the site of genomic integration. In certain embodiments, the transgene with homologous nucleotide extensions enable genomic integration by homologous recombination, microhomology-mediated end joining, or nonhomologous end-joining. In certain embodiments the site-specific integration occurs at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C-C motif) receptor 5 (CCRS) gene and the site of the human ortholog of the mouse Rosa26 locus.

In certain embodiments, the site-specific transgene integration occurs at a site that disrupts expression of a target gene. In certain embodiments, disruption of target gene expression occurs by site-specific integration at introns, exons, promoters, genetic elements, enhancers, suppressors, start codons, stop codons, and response elements. In certain embodiments, exemplary target genes targeted by site-specific integration include but are not limited to TRAC, TRAB, PDI, any immunosuppressive gene, and genes involved in allo-rejection.

In certain embodiments, the site-specific transgene integration occurs at a site that results in enhanced expression of a target gene. In certain embodiments, enhancement of target gene expression occurs by site-specific integration at introns, exons, promoters, genetic elements, enhancers, suppressors, start codons, stop codons, and response elements.

In certain embodiments of the methods of the disclosure, enzymes may be used to create strand breaks in the host genome to facilitate delivery or integration of the transgene. In certain embodiments, enzymes create single-strand breaks. In certain embodiments, enzymes create double-strand breaks. In certain embodiments, examples of break-inducing enzymes include but are not limited to: transposases, integrases, endonucleases, CRISPR-Cas9, transcription activator-like effector nucleases (TALEN), zinc finger nucleases (ZFN), Cas-CLOVER™, and cpfl. In certain embodiments, break-inducing enzymes can be delivered to the cell encoded in DNA, encoded in mRNA, as a protein, as a nucleoprotein complex with a guide RNA (gRNA).

In certain embodiments of the methods of the disclosure, the site-specific transgene integration is controlled by a vector-mediated integration site bias. In certain embodiments vector-mediated integration site bias is controlled by the chosen lentiviral vector. In certain embodiments vector-mediated integration site bias is controlled by the chosen gamma-retroviral vector.

In certain embodiments of the methods of the disclosure, the site-specific transgene integration site is a non-stable chromosomal insertion. In certain embodiments, the integrated transgene may become silenced, removed, excised, or further modified. In certain embodiments of the methods of the disclosure, the genome modification is a non-stable integration of a transgene. In certain embodiments, the non-stable integration can be a transient non-chromosomal integration, a semi-stable non chromosomal integration, a semi-persistent non-chromosomal insertion, or a non-stable chromosomal insertion. In certain embodiments, the transient non-chromosomal insertion can be epi-chromosomal or cytoplasmic. In certain embodiments, the transient non-chromosomal insertion of a transgene does not integrate into a chromosome and the modified genetic material is not replicated during cell division.

In certain embodiments of the methods of the disclosure, the genome modification is a semi-stable or persistent non-chromosomal integration of a transgene. In certain embodiments, a DNA vector encodes a Scaffold/matrix attachment region (S-MAR) module that binds to nuclear matrix proteins for episomal retention of a non-viral vector allowing for autonomous replication in the nucleus of dividing cells.

In certain embodiments of the methods of the disclosure, the genome modification is a non-stable chromosomal integration of a transgene. In certain embodiments, the integrated transgene may become silenced, removed, excised, or further modified.

In certain embodiments of the methods of the disclosure, the modification to the genome by transgene insertion can occur via host cell-directed double-strand breakage repair (homology-directed repair) by homologous recombination (HR), microhomology-mediated end joining (MMEJ), nonhomologous end joining (NHEJ), transposase enzyme-mediated modification, integrase enzyme-mediated modification, endonuclease enzyme-mediated modification, or recombinant enzyme-mediated modification. In certain embodiments, the modification to the genome by transgene insertion can occur via CRISPR-Cas9, TALEN, ZFNs, Cas-CLOVER, and cpfl.

In certain embodiments of the methods of the disclosure, a cell with an in vivo or ex vivo genomic modification can be a germline cell or a somatic cell. In certain embodiments the modified cell can be a human, non-human, mammalian, rat, mouse, or dog cell. In certain embodiments, the modified cell can be differentiated, undifferentiated, or immortalized. In certain embodiments, the modified undifferentiated cell can be a stem cell. In certain embodiments, the modified cell can be differentiated, undifferentiated, or immortalized. In certain embodiments, the modified undifferentiated cell can be an induced pluripotent stem cell. In certain embodiments, the modified cell can be a T cell, a hematopoietic stem cell, a natural killer cell, a macrophage, a dendritic cell, a monocyte, a megakaryocyte, or an osteoclast. In certain embodiments, the modified cell can be modified while the cell is quiescent, in an activated state, resting, in interphase, in prophase, in metaphase, in anaphase, or in telophase. In certain embodiments, the modified cell can be fresh, cryopreserved, bulk, sorted into sub-populations, from whole blood, from leukapheresis, or from an immortalized cell line.

### Centyrins

Centyrins of the disclosure may be derived from a fibronectin type III (FN3) repeat protein, encoding or complementary nucleic acids, vectors, host cells, compositions, combinations, formulations, devices, and methods of making and using them. In a preferred embodiment, the Centyrin is comprised of a consensus sequence of multiple FN3 domains from human Tenascin-C (hereinafter “Tenascin”). In a further preferred embodiment, the protein scaffold of the present invention is a consensus sequence of 15 FN3 domains. The Centyrins of the disclosure can be designed to bind various molecules, for example, a cellular target protein. In a preferred embodiment, the Centyrins of the disclosure can be designed to bind an epitope of a wild type and/or variant form of an antigen.

Centyrins of the disclosure may include additional molecules or moieties, for example, the Fc region of an antibody, albumin binding domain, or other moiety influencing half-life. In further embodiments, the Centyrins of the disclosure may be bound to a nucleic acid molecule that may encode the Centyrin.

The disclosure provides at least one method for expressing at least one Centyrin based on a consensus sequence of multiple FN3 domains, in a host cell, comprising culturing a host cell as described herein under conditions wherein at least one protein scaffold is expressed in detectable and/or recoverable amounts.

The disclosure provides at least one composition comprising (a) a Centyrin based on a consensus sequence of multiple FN3 domains and/or encoding nucleic acid as described herein; and (b) a suitable and/or pharmaceutically acceptable carrier or diluent.

The disclosure provides a method of generating libraries of a Centyrin based on a fibronectin type III (FN3) repeat protein, preferably, a consensus sequence of multiple FN3 domains and, more preferably, a consensus sequence of multiple FN3 domains from human Tenascin. The library is formed by making successive generations of Centyrins by altering (by mutation) the amino acids or the number of amino acids in the molecules in particular positions in portions of the Centyrin, e.g., loop regions. Libraries can be generated by altering the amino acid composition of a single loop or the simultaneous alteration of multiple loops or additional positions of the Centyrin molecule. The loops that are altered can be lengthened or shortened accordingly. Such libraries can be generated to include all possible amino acids at each position, or a designed subset of amino acids. The library members can be used for screening by display, such as in vitro or CIS display (DNA, RNA, ribosome display, etc.), yeast, bacterial, and phage display.

Centyrins of the disclosure provide enhanced biophysical properties, such as stability under reducing conditions and solubility at high concentrations; they may be expressed and folded in prokaryotic systems, such as E. coli, in eukaryotic systems, such as yeast, and in in vitro transcription/translation systems, such as the rabbit reticulocyte lysate system.

The disclosure provides a method of generating a Centyrin molecule that binds to a particular target by panning the Centyrin library of the invention with the target and detecting binders. In other related aspects, the disclosure comprises screening methods that may be used to generate or affinity mature Centyrins with the desired activity, e.g., capable of binding to target proteins with a certain affinity. Affinity maturation can be accomplished by iterative rounds of mutagenesis and selection using systems, such as phage display or in vitro display. Mutagenesis during this process may be the result of site directed mutagenesis to specific Centyrin residues, random mutagenesis due to error-prone PCR, DNA shuffling, and/or a combination of these techniques.

The disclosure provides an isolated, recombinant and/or synthetic Centyrin based on a consensus sequence of fibronectin type III (FN3) repeat protein, including, without limitation, mammalian-derived Centyrins, as well as compositions and encoding nucleic acid molecules comprising at least one polynucleotide encoding a Centyrin based on the consensus FN3 sequence. The disclosure further includes, but is not limited to, methods of making and using such nucleic acids and Centyrins, including diagnostic and therapeutic compositions, methods and devices.

The Centyrins of the disclosure offer advantages over conventional therapeutics, such as ability to administer locally, orally, or cross the blood-brain barrier, ability to express in E. Coli allowing for increased expression of protein as a function of resources versus mammalian cell expression ability to be engineered into bispecific or tandem molecules that bind to multiple targets or multiple epitopes of the same target, ability to be conjugated to drugs, polymers, and probes, ability to be formulated to high concentrations, and the ability of such molecules to effectively penetrate diseased tissues and tumors.

Moreover, the Centyrins possess many of the properties of antibodies in relation to their fold that mimics the variable region of an antibody. This orientation enables the FN3 loops to be exposed similar to antibody complementarity determining regions (CDRs). They should be able to bind to cellular targets and the loops can be altered, e.g., affinity matured, to improve certain binding or related properties.

Three of the six loops of the Centyrin of the disclosure correspond topologically to the complementarity determining regions (CDRs 1-3), i.e., antigen-binding regions, of an antibody, while the remaining three loops are surface exposed in a manner similar to antibody CDRs. These loops span at or about residues 13-16, 22-28, 38-43, 51-54, 60-64, and 75-81 of SEQ ID NO: 18018. Preferably, the loop regions at or about residues 22-28, 51-54, and 75-81 of SEQ ID NO: 18018 are altered for binding specificity and affinity. One or more of these loop regions are randomized with other loop regions and/or other strands maintaining their sequence as backbone portions to populate a library and potent binders can be selected from the library having high affinity for a particular protein target. One or more of the loop regions can interact with a target protein similar to an antibody CDR interaction with the protein.

In certain embodiments of the disclosure, PSMA-specific Centyrins are designed, evolved and/or selected for their ability to specifically bind to a sequence of PSMA. In certain embodiments, the PSMA-specific Centyrins are capable of binding to a sequence of PSMA with a comparable affinity to that of an anti-PSMA antibody binding an epitope of PSMA. In certain embodiments, the PSMA-specific Centyrins are capable of binding to a sequence of PSMA with a stronger affinity to that of an anti-PSMA antibody binding an epitope of PSMA. In certain embodiments, the PSMA-specific Centyrins are capable of binding to a sequence of PSMA to which an antibody is not capable of binding. For example the sequence of PSMA may be discontinuous or may have a secondary structure.

### Production and Generation of CARTyrins

At least one CARTyrin of the disclosure can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

Amino acids from a CARTyrin can be altered, added and/or deleted to reduce immunogenicity or reduce, enhance or modify binding, affinity, on-rate, off-rate, avidity, specificity, half-life, stability, solubility or any other suitable characteristic, as known in the art.

Optionally, CARTyrins can be engineered with retention of high affinity for the antigen and other favorable biological properties. To achieve this goal, the CARTyrins can be optionally prepared by a process of analysis of the parental sequences and various conceptual engineered products using three-dimensional models of the parental and engineered sequences. Three-dimensional models are commonly available and are familiar to those skilled in the art. Computer programs are available which illustrate and display probable three-dimensional conformational structures of selected candidate sequences and can measure possible immunogenicity (e.g., Immunofilter program of Xencor, Inc. of Monrovia, Calif.). Inspection of these displays permits analysis of the likely role of the residues in the functioning of the candidate sequence, i.e., the analysis of residues that influence the ability of the candidate Centyrin or CARTyrin to bind its antigen. In this way, residues can be selected and combined from the parent and reference sequences so that the desired characteristic, such as affinity for the target antigen(s), is achieved. Alternatively, or in addition to, the above procedures, other suitable methods of engineering can be used.

### Screening of CARTyrin Proteins

Screening Centyrins or CARTyrins for specific binding to similar proteins or fragments can be conveniently achieved using nucleotide (DNA or RNA display) or peptide display libraries, for example, in vitro display. This method involves the screening of large collections of peptides for individual members having the desired function or structure. The displayed nucleotide or peptide sequences can be from 3 to 5000 or more nucleotides or amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. The Centyrins CARTyrins of the disclosure can bind human or other mammalian proteins with a wide range of affinities (KD). In a preferred embodiment, at least one Centyrin of the present invention can optionally bind to a target protein with high affinity, for example, with a KD equal to or less than about 10⁻⁷ M, such as but not limited to, 0.1-9.9 (or any range or value therein)×10⁻⁸, 10⁻⁹, 10⁻¹⁰, 10⁻¹¹, 10⁻¹², 10⁻¹³, 10⁻¹⁴, 10⁻¹⁵ or any range or value therein, as determined by surface plasmon resonance or the Kinexa method, as practiced by those of skill in the art.

The affinity or avidity of a Centyrin or CARTyrin for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W.H. Freeman and Company: New York, N.Y. (1992); and methods described herein). The measured affinity of a particular Centyrin-antigen or Centyrin-antigen interaction can vary if measured under different conditions (e.g., salt concentration, pH). Thus, measurements of affinity and other antigen-binding parameters (e.g., KD, Kon, Koff) are preferably made with standardized solutions of Centyrins or CARTyrins and antigen, and a standardized buffer, such as the buffer described herein.

Competitive assays can be performed with the Centyrin or CARTyrin of the disclosure in order to determine what proteins, antibodies, and other antagonists compete for binding to a target protein with the Centyrin or CARTyrin of the present invention and/or share the epitope region. These assays as readily known to those of ordinary skill in the art evaluate competition between antagonists or ligands for a limited number of binding sites on a protein. The protein and/or antibody is immobilized or insolubilized before or after the competition and the sample bound to the target protein is separated from the unbound sample, for example, by decanting (where the protein/antibody was preinsolubilized) or by centrifuging (where the protein/antibody was precipitated after the competitive reaction). Also, the competitive binding may be determined by whether function is altered by the binding or lack of binding of the Centyrin or CARTyrin to the target protein, e.g., whether the Centyrin or CARTyrin molecule inhibits or potentiates the enzymatic activity of, for example, a label. ELISA and other functional assays may be used, as well known in the art.

### Nucleic Acid Molecules

Nucleic acid molecules of the disclosure encoding Centyrins or CARTyrins can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the disclosure can include nucleic acid molecules comprising an open reading frame (ORF), optionally, with one or more introns, e.g., but not limited to, at least one specified portion of at least one CARTyrin; nucleic acid molecules comprising the coding sequence for a CARTyrin; and nucleic acid molecules which comprise a nucleotide sequence substantially different from those described above but which, due to the degeneracy of the genetic code, still encode the CARTyrin as described herein and/or as known in the art. Of course, the genetic code is well known in the art. Thus, it would be routine for one skilled in the art to generate such degenerate nucleic acid variants that code for specific CARTyrins of the present invention. See, e.g., Ausubel, et al., supra, and such nucleic acid variants are included in the present invention.

As indicated herein, nucleic acid molecules of the disclosure which comprise a nucleic acid encoding a CARTyrin can include, but are not limited to, those encoding the amino acid sequence of a Centyrin fragment, by itself the coding sequence for the entire CARTyrin or a portion thereof the coding sequence for a Centyrin, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding a CARTyrin can be fused to a marker sequence, such as a sequence encoding a peptide that facilitates purification of the fused CARTyrin comprising a Centyrin fragment or portion.

### Polynucleotides Selectively Hybridizing to a Polynucleotide as Described Herein

The disclosure provides isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides of this invention will encode at least a portion of a CARTyrin encoded by the polynucleotides described herein. The polynucleotides of this invention embrace nucleic acid sequences that can be employed for selective hybridization to a polynucleotide encoding a CARTyrin of the present invention. See, e.g., Ausubel, supra; Colligan, supra, each entirely incorporated herein by reference.

### Construction of Nucleic Acids

The isolated nucleic acids of the disclosure can be made using (a) recombinant methods, (b) synthetic techniques, (c) purification techniques, and/or (d) combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra).

### Recombinant Methods for Constructing Nucleic Acids

The isolated nucleic acid compositions of this disclosure, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries are well known to those of ordinary skill in the art. (See, e.g., Ausubel, supra; or Sambrook, supra).

### Nucleic Acid Screening and Isolation Methods

A cDNA or genomic library can be screened using a probe based upon the sequence of a polynucleotide of the disclosure. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the disclosure without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. Nos. 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The isolated nucleic acids of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The disclosure further provides recombinant expression cassettes comprising a nucleic acid of the disclosure. A nucleic acid sequence of the disclosure, for example, a cDNA or a genomic sequence encoding a CARTyrin of the disclosure, can be used to construct a recombinant expression cassette that can be introduced into at least one desired host cell. A recombinant expression cassette will typically comprise a polynucleotide of the disclosure operably linked to transcriptional initiation regulatory sequences that will direct the transcription of the polynucleotide in the intended host cell. Both heterologous and non-heterologous (i.e., endogenous) promoters can be employed to direct expression of the nucleic acids of the disclosure.

In some embodiments, isolated nucleic acids that serve as promoter, enhancer, or other elements can be introduced in the appropriate position (upstream, downstream or in the intron) of a non-heterologous form of a polynucleotide of the disclosure so as to up or down regulate expression of a polynucleotide of the disclosure. For example, endogenous promoters can be altered in vivo or in vitro by mutation, deletion and/or substitution.

### Nanotransposons

The disclosure provides a nanotransposon comprising: (a) a sequence encoding a transposon insert, comprising a sequence encoding a first inverted terminal repeat (ITR), a sequence encoding a second inverted terminal repeat (ITR), and an intra-ITR sequence; (b) a sequence encoding a backbone, wherein the sequence encoding the backbone comprises a sequence encoding an origin of replication having between 1 and 450 nucleotides, inclusive of the endpoints, and a sequence encoding a selectable marker having between 1 and 200 nucleotides, inclusive of the endpoints, and (c) an inter-ITR sequence. In some embodiments, the inter-ITR sequence of (c) comprises the sequence of (b). In some embodiments, the intra-ITR sequence of (a) comprises the sequence of (b).

In some embodiments of the nanotransposons of the disclosure, the sequence encoding the backbone comprises between 1 and 600 nucleotides, inclusive of the endpoints. In some embodiments, the sequence encoding the backbone consists of between 1 and 50 nucleotides, between 50 and 100 nucleotides, between 100 and 150 nucleotides, between 150 and 200 nucleotides, between 200 and 250 nucleotides, between 250 and 300 nucleotides, between 300 and 350 nucleotides, between 350 and 400 nucleotides, between 400 and 450 nucleotides, between 450 and 500 nucleotides, between 500 and 550 nucleotides, between 550 and 600 nucleotides, each range inclusive of the endpoints.

In some embodiments of the nanotransposons of the disclosure, the inter-ITR sequence comprises between 1 and 1000 nucleotides, inclusive of the endpoints. In some embodiments, the inter-ITR sequence consists of between 1 and 50 nucleotides, between 50 and 100 nucleotides, between 100 and 150 nucleotides, between 150 and 200 nucleotides, between 200 and 250 nucleotides, between 250 and 300 nucleotides, between 300 and 350 nucleotides, between 350 and 400 nucleotides, between 400 and 450 nucleotides, between 450 and 500 nucleotides, between 500 and 550 nucleotides, between 550 and 600 nucleotides, between 600 and 650 nucleotides, between 650 and 700 nucleotides, between 700 and 750 nucleotides, between 750 and 800 nucleotides, between 800 and 850 nucleotides, between 850 and 900 nucleotides, between 900 and 950 nucleotides, or between 950 and 1000 nucleotides, each range inclusive of the endpoints.

In some embodiments of the nanotransposons of the disclosure, including the short nanotransposons (SNTs) of the disclosure, the inter-ITR sequence comprises between 1 and 200 nucleotides, inclusive of the endpoints. In some embodiments, the inter-ITR sequence consists of between 1 and 10 nucleotides, between 10 and 20 nucleotides, between 20 and 30 nucleotides, between 30 and 40 nucleotides, between 40 and 50 nucleotides, between 50 and 60 nucleotides, between 60 and 70 nucleotides, between 70 and 80 nucleotides, between 80 and 90 nucleotides, or between 90 and 100 nucleotides, each range inclusive of the endpoints.

In some embodiments of the nanotransposons of the disclosure, the selectable marker having between 1 and 200 nucleotides, inclusive of the endpoints, comprises a sequence encoding a sucrose-selectable marker. In some embodiments, the sequence encoding a sucrose-selectable marker comprises a sequence encoding an RNA-OUT sequence. In some embodiments, the sequence encoding an RNA-OUT sequence comprises or consists of 137 base pairs (bp). In some embodiments, the selectable marker having between 1 and 200 nucleotides, inclusive of the endpoints, comprises a sequence encoding a fluorescent marker. In some embodiments, the selectable marker having between 1 and 200 nucleotides, inclusive of the endpoints, comprises a sequence encoding a cell surface marker.

In some embodiments of the nanotransposons of the disclosure, the sequence encoding an origin of replication having between 1 and 450 nucleotides, inclusive of the endpoints, comprises a sequence encoding a mini origin of replication. In some embodiments, the sequence encoding an origin of replication having between 1 and 450 nucleotides, inclusive of the endpoints, comprises a sequence encoding an R6K origin of replication. In some embodiments, the R6K origin of replication comprises an R6K gamma origin of replication. In some embodiments, the R6K origin of replication comprises an R6K mini origin of replication. In some embodiments, the R6K origin of replication comprises an R6K gamma mini origin of replication. In some embodiments, the R6K gamma mini origin of replication comprises or consists of 281 base pairs (bp).

In some embodiments of the nanotransposons of the disclosure, the sequence encoding the backbone does not comprise a recombination site, an excision site, a ligation site or a combination thereof. In some embodiments, neither the nanotransposon nor the sequence encoding the backbone comprises a product of a recombination site, an excision site, a ligation site or a combination thereof. In some embodiments, neither the nanotransposon nor the sequence encoding the backbone is derived from a recombination site, an excision site, a ligation site or a combination thereof.

In some embodiments of the nanotransposons of the disclosure, a recombination site comprises a sequence resulting from a recombination event. In some embodiments, a recombination site comprises a sequence that is a product of a recombination event. In some embodiments, the recombination event comprises an activity of a recombinase (e.g., a recombinase site).

In some embodiments of the nanotransposons of the disclosure, the sequence encoding the backbone does not further comprise a sequence encoding foreign DNA.

In some embodiments of the nanotransposons of the disclosure, the inter-ITR sequence does not comprise a recombination site, an excision site, a ligation site or a combination thereof. In some embodiments, the inter-ITR sequence does not comprise a product of a recombination event, an excision event, a ligation event or a combination thereof. In some embodiments, the inter-ITR sequence is not derived from a recombination event, an excision event, a ligation event or a combination thereof.

In some embodiments of the nanotransposons of the disclosure, the inter-ITR sequence comprises a sequence encoding foreign DNA.

In some embodiments of the nanotransposons of the disclosure, the intra-ITR sequence comprises at least one sequence encoding an insulator and a sequence encoding a promoter capable of expressing an exogenous sequence in a mammalian cell. In some embodiments, the mammalian cell is a human cell.

In some embodiments of the nanotransposons of the disclosure, the intra-ITR sequence comprises a first sequence encoding an insulator, a sequence encoding a promoter capable of expressing an exogenous sequence in a mammalian cell and a second sequence encoding an insulator.

In some embodiments of the nanotransposons of the disclosure, the intra-ITR sequence comprises a first sequence encoding an insulator, a sequence encoding a promoter capable of expressing an exogenous sequence in a mammalian cell, a polyadenosine (polyA) sequence and a second sequence encoding an insulator.

In some embodiments of the nanotransposons of the disclosure, the intra-ITR sequence comprises a first sequence encoding an insulator, a sequence encoding a promoter capable of expressing an exogenous sequence in a mammalian cell, at least one exogenous sequence, a polyadenosine (polyA) sequence and a second sequence encoding an insulator.

In some embodiments of the nanotransposons of the disclosure, the sequence encoding a promoter capable of expressing an exogenous sequence in a mammalian cell is capable of expressing an exogenous sequence in a human cell. In some embodiments, the sequence encoding a promoter capable of expressing an exogenous sequence in a mammalian cell comprises a sequence encoding a constitutive promoter. In some embodiments, the sequence encoding a promoter capable of expressing an exogenous sequence in a mammalian cell comprises a sequence encoding an inducible promoter. In some embodiments, the intra-ITR sequence comprises a first sequence encoding a first promoter capable of expressing an exogenous sequence in a mammalian cell and a second sequence encoding a second promoter capable of expressing an exogenous sequence in mammalian cell, wherein the first promoter is a constitutive promoter, wherein the second promoter is an inducible promoter, and wherein the first sequence encoding the first promoter and the second sequence encoding the second promoter are oriented in opposite directions. In some embodiments, the sequence encoding a promoter capable of expressing an exogenous sequence in a mammalian cell comprises a sequence encoding a cell-type or tissue-type specific promoter. In some embodiments, the sequence encoding a promoter capable of expressing an exogenous sequence in a mammalian cell comprises a sequence encoding an EF1a promoter, a sequence encoding a CMV promoter, a sequence encoding an MND promoter, a sequence encoding an SV40 promoter, a sequence encoding a PGK1 promoter, a sequence encoding a Ubc promoter, a sequence encoding a CAG promoter, a sequence encoding an H1 promoter, or a sequence encoding a U6 promoter.

In some embodiments of the nanotransposons of the disclosure, the polyadenosine (polyA) sequence is isolated or derived from a viral polyA sequence. In some embodiments, the polyadenosine (polyA) sequence is isolated or derived from an (SV40) polyA sequence.

In some embodiments of the nanotransposons of the disclosure, the at least one exogenous sequence comprises an inducible proapoptotic polypeptide. In some embodiments, the inducible caspase polypeptide comprises (a) a ligand binding region, (b) a linker, and (c) a caspase polypeptide, wherein the inducible proapoptotic polypeptide does not comprise a non-human sequence. In some embodiments, the inducible caspase polypeptide comprises (a) a ligand binding region, (b) a linker, and (c) a truncated caspase 9 polypeptide, wherein the inducible proapoptotic polypeptide does not comprise a non-human sequence.

In some embodiments of the nanotransposons of the disclosure, including those wherein the at least one exogenous sequence comprises an inducible proapoptotic polypeptide, the ligand binding region comprises a FK506 binding protein 12 (FKBP12) polypeptide. In some embodiments, the amino acid sequence of the ligand binding region comprises a FK506 binding protein 12 (FKBP12) polypeptide. In some embodiments, the FK506 binding protein 12 (FKBP12) polypeptide comprises a modification at position 36 of the sequence. In some embodiments, the modification comprises a substitution of valine (V) for phenylalanine (F) at position 36 (F36V). In some embodiments, the FKBP12 polypeptide is encoded by an amino acid sequence comprising

(SEQ ID NO: 14635)
GVQVETISPGDGRTFPKRGQTCVVHYTGMLEDGKKVDSSRDRNKPFKFML
GKQEVIRGWEEGVAQMSVGQRAKLTISPDYAYGATGHPGIIPPHATLVFD
VELLKLE.

In some embodiments, the FKBP12 polypeptide is encoded by a nucleic acid sequence comprising

(SEQ ID NO: 14636)
GGGGTCCAGGTCGAGACTATTTCACCAGGGGATGGGCGAACATTTCCAAA
AAGGGGCCAGACTTGCGTCGTGCATTACACCGGGATGCTGGAGGACGGGA
AGAAAGTGGACAGCTCCAGGGATCGCAACAAGCCCTTCAAGTTCATGCTG
GGAAAGCAGGAAGTGATCCGAGGATGGGAGGAAGGCGTGGCACAGATGTC
AGTCGGCCAGCGGGCCAAACTGACCATTAGCCCTGACTACGCTTATGGAG
CAACAGGCCACCCAGGGATCATTCCCCCTCATGCCACCCTGGTCTTCGAT
GTGGAACTGCTGAAGCTGGAG.

In some embodiments of the nanotransposons of the disclosure, including those wherein the at least one exogenous sequence comprises an inducible proapoptotic polypeptide, the linker region is encoded by an amino acid comprising GGGGS (SEQ ID NO: 14637) or a nucleic acid sequence comprising GGAGGAGGAGGATCC (SEQ ID NO: 14638). In some embodiments, the nucleic acid sequence encoding the linker does not comprise a restriction site.

In some embodiments of the nanotransposons of the disclosure, including those wherein the at least one exogenous sequence comprises an inducible proapoptotic polypeptide, the truncated caspase 9 polypeptide is encoded by an amino acid sequence that does not comprise an arginine (R) at position 87 of the sequence. In some embodiments, the truncated caspase 9 polypeptide is encoded by an amino acid sequence that does not comprise an alanine (A) at position 282 the sequence. In some embodiments, the truncated caspase 9 polypeptide is encoded by an amino acid comprising

(SEQ ID NO: 14639)
GFGDVGALESLRGNADLAYILSMEPCGHCLIINNVNFCRESGLRTRTGSN
IDCEKLRRRFSSLHFMVEVKGDLTAKKMVLALLELAQQDHGALDCCVVVI
LSHGCQASHLQFPGAVYGTDGCPVSVEKIVNIFNGTSCPSLGGKPKLFFI
QACGGEQKDHGFEVASTSPEDESPGSNPEPDATPFQEGLRTFDQLDAISS
LPTPSDIFVSYSTFPGFVSWRDPKSGSWYVETLDDIFEQWAHSEDLQSLL
LRVANAVSVKGIYKQMPGCFNFLRKKLFFKTS.

In some embodiments, the truncated caspase 9 polypeptide is encoded by a nucleic acid sequence comprising

(SEQ ID NO: 14640)
GGATTTGGGGACGTGGGGGCCCTGGAGTCTCTGCGAGGAAATGCCGATCT
GGCTTACATCCTGAGCATGGAACCCTGCGGCCACTGTCTGATCATTAACA
ATGTGAACTTCTGCAGAGAAAGCGGACTGCGAACACGGACTGGCTCCAAT
ATTGACTGTGAGAAGCTGCGGAGAAGGTTCTCTAGTCTGCACTTTATGGT
CGAAGTGAAAGGGGATCTGACCGCCAAGAAAATGGTGCTGGCCCTGCTGG
AGCTGGCTCAGCAGGACCATGGAGCTCTGGATTGCTGCGTGGTCGTGATC
CTGTCCCACGGGTGCCAGGCTTCTCATCTGCAGTTCCCCGGAGCAGTGTA
CGGAACAGACGGCTGTCCTGTCAGCGTGGAGAAGATCGTCAACATCTTCA
ACGGCACTTCTTGCCCTAGTCTGGGGGGAAAGCCAAAACTGTTCTTTATC
CAGGCCTGTGGCGGGGAACAGAAAGATCACGGCTTCGAGGTGGCCAGCAC
CAGCCCTGAGGACGAATCACCAGGGAGCAACCCTGAACCAGATGCAACTC
CATTCCAGGAGGGACTGAGGACCTTTGACCAGCTGGATGCTATCTCAAGC
CTGCCCACTCCTAGTGACATTTTCGTGTCTTACAGTACCTTCCCAGGCTT
TGTCTCATGGCGCGATCCCAAGTCAGGGAGCTGGTACGTGGAGACACTGG
ACGACATCTTTGAACAGTGGGCCCATTCAGAGGACCTGCAGAGCCTGCTG
CTGCGAGTGGCAAACGCTGTCTCTGTGAAGGGCATCTACAAACAGATGCC
CGGGTGCTTCAATTTTCTGAGAAAGAAACTGTTCTTTAAGACTTCC.

In some embodiments of the nanotransposons of the disclosure, including those wherein the at least one exogenous sequence comprises an inducible proapoptotic polypeptide, the inducible proapoptotic polypeptide is encoded by an amino acid sequence comprising

(SEQ ID NO: 14641)
GVQVETISPGDGRTFPKRGQTCVVHYTGMLEDGKKVDSSRDRNKPFKFML
GKQEVIRGWEEGVAQMSVGQRAKLTISPDYAYGATGHPGIIPPHATLVFD
VELLKLEGGGGSGFGDVGALESLRGNADLAYILSMEPCGHCLIINNVNFC
RESGLRTRTGSNIDCEKLRRRFSSLHFMVEVKGDLTAKKMVLALLELAQQ
DHGALDCCVVVILSHGCQASHLQFPGAVYGTDGCPVSVEKIVNIFNGTSC
PSLGGKPKLFFIQACGGEQKDHGFEVASTSPEDESPGSNPEPDATPFQEG
LRTFDQLDAISSLPTPSDIFVSYSTFPGFVSWRDPKSGSWYVETLDDIFE
QWAHSEDLQSLLLRVANAVSVKGIYKQMPGCFNFLRKKLFFKTS.

In some embodiments, the inducible proapoptotic polypeptide is encoded by a nucleic acid sequence comprising

(SEQ ID NO: 14642)
ggggtccaggtcgagactatttcaccaggggatgggcgaacatttccaaa
aaggggccagacttgcgtcgtgcattacaccgggatgctggaggacggga
agaaagtggacagctccagggatcgcaacaagcccttcaagttcatgctg
ggaaagcaggaagtgatccgaggatgggaggaaggcgtggcacagatgtc
agtcggccagcgggccaaactgaccattagccctgactacgcttatggag
caacaggccacccagggatcattccccctcatgccaccctggtcttcgat
gtggaactgctgaagctggagggaggaggaggatccggatttggggacgt
gggggccctggagtctctgcgaggaaatgccgatctggcttacatcctga
gcatggaaccctgcggccactgtctgatcattaacaatgtgaacttctgc
agagaaagcggactgcgaacacggactggctccaatattgactgtgagaa
gctgcggagaaggttctctagtctgcactttatggtcgaagtgaaagggg
atctgaccgccaagaaaatggtgctggccctgctggagctggctcagcag
gaccatggagctctggattgctgcgtggtcgtgatcctgtcccacgggtg
ccaggcttctcatctgcagttccccggagcagtgtacggaacagacggct
gtcctgtcagcgtggagaagatcgtcaacatcttcaacggcacttcttgc
cctagtctggggggaaagccaaaactgttctttatccaggcctgtggcgg
ggaacagaaagatcacggcttcgaggtggccagcaccagccctgaggacg
aatcaccagggagcaaccctgaaccagatgcaactccattccaggaggga
ctgaggacctttgaccagctggatgctatctcaagcctgcccactcctag
tgacattacgtgtcttacagtaccttcccaggctttgtctcatggcgcga
tcccaagtcagggagctggtacgtggagacactggacgacatctttgaac
agtgggcccattcagaggacctgcagagcctgctgctgcgagtggcaaac
gctgtctctgtgaagggcatctacaaacagatgcccgggtgcttcaatta
ctgagaaagaaactgttctttaagacttcc.

In some embodiments of the nanotransposons of the disclosure, including those wherein the at least one exogenous sequence comprises an inducible proapoptotic polypeptide, the exogenous sequence further comprises a sequence encoding a selectable marker. In some embodiments, the sequence encoding the selectable marker comprises a sequence encoding a detectable marker. In some embodiments, the detectable marker comprises a fluorescent marker or a cell-surface marker. In some embodiments, the sequence encoding the selectable marker comprises a sequence encoding a protein that is active in dividing cells and not active in non-dividing cells. In some embodiments, the sequence encoding the selectable marker comprises a sequence encoding a metabolic marker. In some embodiments, the sequence encoding the selectable marker comprises a sequence encoding a dihydrofolate reductase (DHFR) mutein enzyme. In some embodiments, the DHFR mutein enzyme comprises or consists of the amino acid sequence of:

```
(SEQ ID NO: 17012)
    1 MVGSLNCIVA VSQNMGIGKN GDFPWPPLRN ESRYFQRMTT TSSVEGKQNL
  61 VIMGKKTWFS IPEKNRPLKG RINLVLSREL KEPPQGAHFL SRSLDDALKL
121 TEQPELANKV DMVWIVGGSS VYKEAMNHPG HLKLFVTRIM QDFESDTFFP
181 EIDLEKYKLL PEYPGVLSDV QEEKGIKYKF EVYEKND.
```

In some embodiments, the DHFR mutein enzyme is encoded by a the nucleic acid sequence comprising or consisting of

(SEQ ID NO: 17095)
atggtcgggtctctgaattgtatcgtcgccgtgagtcagaacatgggcat
tgggaagaatggcgatttcccatggccacctctgcgcaacgagtcccgat
actttcagcggatgacaactacctcctctgtggaagggaaacagaatctg
gtcatcatgggaaagaaaacttggttcagcattccagagaagaaccggcc
cctgaaaggcagaatcaatctggtgctgtcccgagaactgaaggagccac
cacagggagctcactttctgagccggtccctggacgatgcactgaagctg
acagaacagcctgagctggccaacaaagtcgatatggtgtggatcgtcgg
gggaagttcagtgtataaggaggccatgaatcaccccggccatctgaaac
tgttcgtcacacggatcatgcaggactttgagagcgatactttctttcct
gaaattgacctggagaagtacaaactgctgcccgaatatcctggcgtgct
gtccgatgtccaggaagagaaaggcatcaaatacaagttcgaggtctatg
agaagaatgac.

In some embodiments, the amino acid sequence of the DHFR mutein enzyme further comprises a mutation at one or more of positions 80, 113, or 153. In some embodiments, the amino acid sequence of the DHFR mutein enzyme comprises one or more of a substitution of a Phenylalanine (F) or a Leucine (L) at position 80, a substitution of a Leucine (L) or a Valine (V) at position 113, and a substitution of a Valine (V) or an Aspartic Acid (D) at position 153.

In some embodiments of the nanotransposons of the disclosure, including those wherein the at least one exogenous sequence comprises an inducible proapoptotic polypeptide and/or the exogenous sequence comprises a sequence encoding a selectable marker, the exogenous sequence further comprises a sequence encoding a non-naturally occurring antigen receptor, and/or a sequence encoding a therapeutic polypeptide. In some embodiments, the non-naturally occurring antigen receptor comprises a T cell Receptor (TCR). In some embodiments, a sequence encoding the TCR comprises one or more of an insertion, a deletion, a substitution, an invertion, a transposition or a frameshift compared to a corresponding wild type sequence. In some embodiments, a sequence encoding the TCR comprises a chimeric or recombinant sequence. In some embodiments, the non-naturally occurring antigen receptor comprises a chimeric antigen receptor (CAR). In some embodiments, the CAR comprises: (a) an ectodomain comprising an antigen recognition region, (b) a transmembrane domain, and (c) an endodomain comprising at least one costimulatory domain. In some embodiments, the ectodomain of (a) of the CAR further comprises a signal peptide. In some embodiments, the ectodomain of (a) of the CAR further comprises a hinge between the antigen recognition region and the transmembrane domain. In some embodiments, the endodomain comprises a human CD3ζ endodomain. In some embodiments, the at least one costimulatory domain comprises a human 4-1BB, CD28, CD40, ICOS, MyD88, OX-40 intracellular segment, or any combination thereof. In some embodiments, the at least one costimulatory domain comprises a human CD28 and/or a 4-1BB costimulatory domain. In some embodiments, the antigen recognition region comprises one or more of a scFv, a VHH, a VH, and a Centyrin.

In some embodiments of the nanotransposons of the disclosure, including those wherein the at least one exogenous sequence comprises an inducible proapoptotic polypeptide and/or the exogenous sequence comprises a sequence encoding a selectable marker, the exogenous sequence further comprises a sequence encoding a transposase.

In some embodiments of the nanotransposons of the disclosure, the intra-ITR sequence comprises a sequence encoding a selectable marker, an exogenous sequence, a sequence encoding an inducible caspase polypeptide, and at least one sequence encoding a self-cleaving peptide. In some embodiments, the at least one sequence encoding a self-cleaving peptide is positioned between one or more of: (a) the sequence encoding a selectable marker and the exogenous sequence, (b) the sequence encoding a selectable marker and the inducible caspase polypeptide, and (c) the exogenous sequence and the inducible caspase polypeptide. In some embodiments, a first sequence encoding a self-cleaving peptide is positioned between the sequence encoding a selectable marker and the exogenous sequence and a second sequence encoding a self-cleaving peptide is positioned between the exogenous sequence and the inducible caspase polypeptide.

In some embodiments of the nanotransposons of the disclosure, including those wherein the at least one exogenous sequence comprises one or more of an inducible proapoptotic polypeptide, a sequence encoding a selectable marker, and an exogenous sequence, the sequence encoding a first inverted terminal repeat (ITR) or the sequence encoding a second inverted terminal repeat (ITR) are recognized by a piggyBac transposase or a piggyBac-like transposase. In some embodiments, the sequence encoding a first inverted terminal repeat (ITR) or the sequence encoding a second inverted terminal repeat (ITR) are recognized by a piggyBac transposase. In some embodiments, the sequence encoding a first inverted terminal repeat (ITR) or the sequence encoding a second inverted terminal repeat (ITR) are recognized by a piggyBac-like transposase. In some embodiments, the sequence encoding a first inverted terminal repeat (ITR) or the sequence encoding a second inverted terminal repeat (ITR) comprise a TTAA, a TTAT or a TTAX recognition sequence. In some embodiments, the sequence encoding a first inverted terminal repeat (ITR) or the sequence encoding a second inverted terminal repeat (ITR) comprise a TTAA, a TTAT or a TTAX recognition sequence and a sequence having at least 50% identity to a sequence isolated or derived from a piggyBac transposase or a piggyBac-like transposase. In some embodiments, the sequence encoding a first inverted terminal repeat (ITR) or the sequence encoding a second inverted terminal repeat (ITR) comprise at least 2 nucleotides (nts), 3 nts, 4 nts, 5 nts, 6 nts, 7 nts, 8 nts, 9 nts, 10 nts, 11 nts, 12 nts, 13 nts, 14 nts, 15 nts, 16 nts, 17 nts, 18 nts, 19 nts, or 20 nts.

In some embodiments of the nanotransposons of the disclosure, including those wherein the at least one exogenous sequence comprises one or more of an inducible proapoptotic polypeptide, a sequence encoding a selectable marker, and an exogenous sequence, the sequence encoding a first inverted terminal repeat (ITR) or the sequence encoding a second inverted terminal repeat (ITR) are recognized by a piggyBac transposase or a piggyBac-like transposase. In some embodiments, the sequence encoding a first inverted terminal repeat (ITR) or the sequence encoding a second inverted terminal repeat (ITR) comprises the sequence of

(SEQ ID NO: 17096)
CCCTAGAAAGATAGTCTGCGTAAAATTGACGCATG

or a sequence having at least 70% identity to the sequence of

(SEQ ID NO: 17096)
CCCTAGAAAGATAGTCTGCGTAAAATTGACGCATG.

In some embodiments, the sequence encoding a first inverted terminal repeat (ITR) or the sequence encoding a second inverted terminal repeat (ITR) comprises the sequence of

(SEQ ID NO: 17097)
CCCTAGAAAGATAATCATATTGTGACGTACGTTAAAGATAATCATGCGTA
AAATTGACGCATG.

In some embodiments, the sequence encoding a first inverted terminal repeat (ITR) or the sequence encoding a second inverted terminal repeat (ITR) comprises the sequence of

(SEQ ID NO: 17096)
CCCTAGAAAGATAGTCTGCGTAAAATTGACGCATG

and comprises the sequence of

(SEQ ID NO: 17097)
CCCTAGAAAGATAATCATATTGTGACGTACGTTAAAGATAATCATGCGTA
AAATTGACGCATG.

In some embodiments, the sequence encoding a first inverted terminal repeat (ITR) or the sequence encoding a second inverted terminal repeat (ITR) comprises the sequence of

(SEQ ID NO: 17096)
CCCTAGAAAGATAGTCTGCGTAAAATTGACGCATG

and comprises the sequence of

(SEQ ID NO: 17098)
CCCTAGAAAGATAATCATATTGTGACGTACGTTAAAGATAATCATGTGTA
AAATTGACGCATG.

In some embodiments, the sequence encoding a first inverted terminal repeat (ITR) or the sequence encoding a second inverted terminal repeat (ITR) comprises the sequence of CCCTAGAAAGATAGTCTGCGTAAAATTGACGCATG (SEQ ID NO: 17096) and comprises the sequence of

(SEQ ID NO: 17099)
TTAACCCTAGAAAGATAATCATATTGTGACGTACGTTAAAGATAATCATG
TGTAAAATTGACGCATGTGTTTTATCGGTCTGTATATCGAGGTTTATTTA
TTAATTTGAATAGATATTAAGTTTTATTATATTTACACTTACATACTAAT
AATAAATTCAACAAACAATTTATTTATGTTTATTTATTTATTAAAAAAAA
CAAAAACTCAAAATTTCTTCTATAAAGTAACAAAACTTTTA.

In some embodiments of the nanotransposons of the disclosure, including those wherein the at least one exogenous sequence comprises one or more of an inducible proapoptotic polypeptide, a sequence encoding a selectable marker, and an exogenous sequence, the sequence encoding a first inverted terminal repeat (ITR) or the sequence encoding a second inverted terminal repeat (ITR) are recognized by a piggyBac transposase or a piggyBac-like transposase. In some embodiments, the sequence encoding a first inverted terminal repeat (ITR) or the sequence encoding a second inverted terminal repeat (ITR) is recognized by a piggyBac transposase having an amino acid sequence of at least 20% identity to the amino acid sequence of

```
(SEQ ID NO: 14487)
    1 MGSSLDDEHI LSALLQSDDE LVGEDSDSEI SDHVSEDDVQ SDTEEAFIDE VHEVQPTSSG
  61 SEILDEQNVI EQPGSSLASN RILTLPQRTI RGKNKHCWST SKSTRRSRVS ALNIVRSQRG
121 PTRMCRNIYD PLLCFKLFFT DEIISEIVKW TNAEISLKRR ESMTGATFRD TNEDEIYAFF
181 GILVMTAVRK DNHMSTDDLF DRSLSMVYVS VMSRDRFDFL IRCLRMDDKS IRPTLRENDV
241 FTPVRKIWDL FIHQCIQNYT PGAHLTIDEQ LLGFRGRCPF RMYIPNKPSK YGIKILMMCD
301 SGTKYMINGM PYLGRGTQTN GVPLGEYYVK ELSKPVHGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDTLD QMCSVMTCSR KTNRWPMALL YGMINIACIN
481 SFIIYSHNVS SKGEKVQSRK KFMRNLYMSL TSSFMRKRLE APTLKRYLRD NISNILPNEV
541 PGTSDDSTEE PVMKKRTYCT YCPSKIRRKA NASCKKCKKV ICREHNIDMC QSCF.
```

In some embodiments, the sequence encoding a first inverted terminal repeat (ITR) or the sequence encoding a second inverted terminal repeat (ITR) is recognized by a piggyBac transposase having the amino acid sequence of

```
(SEQ ID NO: 14487)
    1 MGSSLDDEHI LSALLQSDDE LVGEDSDSEI SDHVSEDDVQ SDTEEAFIDE VHEVQPTSSG
  61 SEILDEQNVI EQPGSSLASN RILTLPQRTI RGKNKHCWST SKSTRRSRVS ALNIVRSQRG
121 PTRMCRNIYD PLLCFKLFFT DEIISEIVKW TNAEISLKRR ESMTGATFRD TNEDEIYAFF
181 GILVMTAVRK DNHMSTDDLF DRSLSMVYVS VMSRDRFDFL IRCLRMDDKS IRPTLRENDV
241 FTPVRKIWDL FIHQCIQNYT PGAHLTIDEQ LLGFRGRCPF RMYIPNKPSK YGIKILMMCD
301 SGTKYMINGM PYLGRGTQTN GVPLGEYYVK ELSKPVHGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDTLD QMCSVMTCSR KTNRWPMALL YGMINIACIN
481 SFIIYSHNVS SKGEKVQSRK KFMRNLYMSL TSSFMRKRLE APTLKRYLRD NISNILPNEV
541 PGTSDDSTEE PVMKKRTYCT YCPSKIRRKA NASCKKCKKV ICREHNIDMC QSCF.
```

In some embodiments, the sequence encoding a first inverted terminal repeat (ITR) or the sequence encoding a second inverted terminal repeat (ITR) is recognized by a piggyBac transposase having an amino acid sequence of at least 20% identity to the amino acid sequence of

```
(SEQ ID NO: 14484)
    1 MGSSLDDEHI LSALLQSDDE LVGEDSDSEV SDHVSEDDVQ SDTEEAFIDE VHEVQPTSSG
  61 SEILDEQNVI EQPGSSLASN RILTLPQRTI RGKNKHCWST SKSTRRSRVS ALNIVRSQRG
121 PTRMCRNIYD PLLCFKLFFT DEIISEIVKW TNAEISLKRR ESMTSATFRD TNEDEIYAFF
181 GILVMTAVRK DNHMSTDDLF DRSLSMVYVS VMSRDRFDFL IRCLRMDDKS IRPTLRENDV
241 FTPVRKIWDL FIHQCIQNYT PGAHLTIDEQ LLGFRGRCPF RVYIPNKPSK YGIKILMMCD
301 SGTKYMINGM PYLGRGTQTN GVPLGEYYVK ELSKPVHGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDTLD QMCSVMTCSR KTNRWPMALL YGMINIACIN
481 SFIIYSHNVS SKGEKVQSRK KFMRNLYMSL TSSFMRKRLE APTLKRYLRD NISNILPKEV
541 PGTSDDSTEE PVMKKRTYCT YCPSKIRRKA NASCKKCKKV ICREHNIDMC QSCF.
```

In some embodiments, the sequence encoding a first inverted terminal repeat (ITR) or the sequence encoding a second inverted terminal repeat (ITR) is recognized by a piggyBac transposase having the amino acid sequence of

```
(SEQ ID NO: 14484)
    1 MGSSLDDEHI LSALLQSDDE LVGEDSDSEV SDHVSEDDVQ SDTEEAFIDE VHEVQPTSSG
  61 SEILDEQNVI EQPGSSLASN RILTLPQRTI RGKNKHCWST SKSTRRSRVS ALNIVRSQRG
121 PTRMCRNIYD PLLCFKLFFT DEIISEIVKW TNAEISLKRR ESMTSATFRD TNEDEIYAFF
181 GILVMTAVRK DNHMSTDDLF DRSLSMVYVS VMSRDRFDFL IRCLRMDDKS IRPTLRENDV
241 FTPVRKIWDL FIHQCIQNYT PGAHLTIDEQ LLGFRGRCPF RVYIPNKPSK YGIKILMMCD
301 SGTKYMINGM PYLGRGTQTN GVPLGEYYVK ELSKPVHGSC RNITCDNWFT SIPLAKNLLQ
361 EPYKLTIVGT VRSNKREIPE VLKNSRSRPV GTSMFCFDGP LTLVSYKPKP AKMVYLLSSC
421 DEDASINEST GKPQMVMYYN QTKGGVDTLD QMCSVMTCSR KTNRWPMALL YGMINIACIN
481 SFIIYSHNVS SKGEKVQSRK KFMRNLYMSL TSSFMRKRLE APTLKRYLRD NISNILPKEV
541 PGTSDDSTEE PVMKKRTYCT YCPSKIRRKA NASCKKCKKV ICREHNIDMC QSCF.
```

In some embodiments of the nanotransposons of the disclosure, including those wherein the at least one exogenous sequence comprises one or more of an inducible proapoptotic polypeptide, a sequence encoding a selectable marker, and an exogenous sequence, the sequence encoding a first inverted terminal repeat (ITR) or the sequence encoding a second inverted terminal repeat (ITR) are recognized by a Sleeping Beauty transposase. In some embodiments, the Sleeping Beauty transposase is a hyperactive Sleeping Beauty transposase (SB100X).

In some embodiments of the nanotransposons of the disclosure, including those wherein the at least one exogenous sequence comprises one or more of an inducible proapoptotic polypeptide, a sequence encoding a selectable marker, and an exogenous sequence, the sequence encoding a first inverted terminal repeat (ITR) or the sequence encoding a second inverted terminal repeat (ITR) are recognized by a Helitron transposase.

In some embodiments of the nanotransposons of the disclosure, including those wherein the at least one exogenous sequence comprises one or more of an inducible proapoptotic polypeptide, a sequence encoding a selectable marker, and an exogenous sequence, the sequence encoding a first inverted terminal repeat (ITR) or the sequence encoding a second inverted terminal repeat (ITR) are recognized by a Tol2 transposase.

The disclosure provides a cell comprising a nanotransposon of the disclosure. In some embodiments, the cell further comprises a transposase composition. In some embodiments, the transposase composition comprises a transposase or a sequence encoding the transposase that is capable of recognizing the first ITR or the second ITR of the nanotransposon. In some embodiments, the transposase composition comprises a nanotransposon comprising the sequence encoding the transposase. In some embodiments, the cell comprises a first nanotransposon comprising an exogenous sequence and a second nanotransposon comprising a sequence encoding a transposase. In some embodiments, the cell is an allogeneic cell.

The disclosure provides a composition comprising the nanotransposon of the disclosure.

The disclosure provides a composition comprising the cell of the disclosure. In some embodiments, the cell comprises a nanotransposon of the disclosure. In some embodiments, the cell is not further modified. In some embodiments, the cell is allogeneic.

The disclosure provides a composition comprising the cell of the disclosure. In some embodiments, the cell comprises a nanotransposon of the disclosure. In some embodiments, the cell is not further modified. In some embodiments, the cell is autologous.

The disclosure provides a composition comprising a plurality of cells of the disclosure. In some embodiments, at least one cell of the plurality of cells comprises a nanotransposon of the disclosure. In some embodiments, a portion of the plurality of cells comprises a nanotransposon of the disclosure. In some embodiments, the portion comprises at least 1%, 2%, 5%, 10%, 15%, 20%, 25%, 30%, 35%, 40%, 45%, 50%, 55%, 60%, 65%, 70%, 75%, 80%, 85%, 90%, 95%, 97%, 99% or any percentage in between of the plurality of cells. In some embodiments, each cell of the plurality of cells comprises a nanotransposon of the disclosure. In some embodiments, the plurality of cells does not comprise a modified cell of the disclosure. In some embodiments, at least one cell of the plurality of cells is not further modified. In some embodiments, none of the plurality of cells is not further modified. In some embodiments, plurality of cells is allogeneic. In some embodiments, an allogeneic plurality of cells are produced according to the methods of the disclosure. In some embodiments, plurality of cells is autologous. In some embodiments, an autologous plurality of cells are produced according to the methods of the disclosure.

The disclosure provides a modified cell comprising: (a) a nanotransposon of the disclosure; (b) a sequence encoding an inducible proapoptotic polypeptide; and wherein the cell is a T cell, (c) a modification of an endogenous sequence encoding a T cell Receptor (TCR), wherein the modification reduces or eliminates a level of expression or activity of the TCR. In some embodiments, the cell further comprises: (d) a non-naturally occurring sequence comprising an HLA class I histocompatibility antigen, alpha chain E (HLA-E), and (e) a modification of an endogenous sequence encoding Beta-2-Microglobulin (B2M), wherein the modification reduces or eliminates a level of expression or activity of a major histocompatibility complex (MHC) class I (MHC-I).

The disclosure provides a modified cell comprising: (a) a nanotransposon of the disclosure; (b) a sequence encoding an inducible proapoptotic polypeptide; (c) a non-naturally occurring sequence comprising an HLA class I histocompatibility antigen, alpha chain E (HLA-E), and (e) a modification of an endogenous sequence encoding Beta-2-Microglobulin (B2M), wherein the modification reduces or eliminates a level of expression or activity of a major histocompatibility complex (MHC) class I (MHC-I).

In some embodiments of the modified cells of the disclosure, the non-naturally occurring sequence comprising a HLA-E further comprises a sequence encoding a B2M signal peptide. In some embodiments, the non-naturally occurring sequence comprising an HLA-E further comprises a linker, wherein the linker is positioned between the sequence encoding the sequence encoding a B2M polypeptide and the sequence encoding the HLA-E. In some embodiments, the non-naturally occurring sequence comprising an HLA-E further comprises a sequence encoding a peptide and a sequence encoding a B2M polypeptide. In some embodiments, the non-naturally occurring sequence comprising an HLA-E further comprises a first linker positioned between the sequence encoding the B2M signal peptide and the sequence encoding the peptide, and a second linker positioned between the sequence encoding the B2M polypeptide and the sequence encoding the HLA-E.

In some embodiments of the cells, unmodified cells and modified cells of the disclosure, the cell is a mammalian cell.

In some embodiments of the cells, unmodified cells and modified cells of the disclosure, the cell is a human cell.

In some embodiments of the cells, unmodified cells and modified cells of the disclosure, the cell is a stem cell.

In some embodiments of the cells, unmodified cells and modified cells of the disclosure, the cell is a differentiated cell.

In some embodiments of the cells, unmodified cells and modified cells of the disclosure, the cell is a somatic cell.

In some embodiments of the cells, unmodified cells and modified cells of the disclosure, the cell is an immune cell or an immune cell precursor. In some embodiments, the immune cell is a lymphoid progenitor cell, a natural killer (NK) cell, a cytokine induced killer (CIK) cell, a T lymphocyte (T cell), a B lymphocyte (B-cell) or an antigen presenting cell (APC). In some embodiments, the immune cell is a T cell, an early memory T cell, a stem cell-like T cell, a stem memory T cell (Tscm), or a central memory T cell (Tcm). In some embodiments, the immune cell precursor is a hematopoietic stem cell (HSC). In some embodiments, the cell is an antigen presenting cell (APC).

In some embodiments of the cells, unmodified cells and modified cells of the disclosure, the cell further comprises a gene editing composition. In some embodiments, the gene editing composition comprises a sequence encoding a DNA binding domain and a sequence encoding a nuclease protein or a nuclease domain thereof. In some embodiments, the gene editing composition comprises a sequence encoding a nuclease protein or a sequence encoding a nuclease domain thereof. In some embodiments, the e sequence encoding a nuclease protein or the sequence encoding a nuclease domain thereof comprises a DNA sequence, an RNA sequence, or a combination thereof. In some embodiments, the nuclease or the nuclease domain thereof comprises one or more of a CRISPR/Cas protein, a Transcription Activator-Like Effector Nuclease (TALEN), a Zinc Finger Nuclease (ZFN), and an endonuclease. In some embodiments, the CRISPR/Cas protein comprises a nuclease-inactivated Cas (dCas) protein.

In some embodiments of the cells, unmodified cells and modified cells of the disclosure, the cell further comprises a gene editing composition. In some embodiments, the gene editing composition comprises a sequence encoding a DNA binding domain and a sequence encoding a nuclease protein or a nuclease domain thereof. In some embodiments, the nuclease or the nuclease domain thereof comprises a nuclease-inactivated Cas (dCas) protein and an endonuclease. In some embodiments, the endonuclease comprises a Clo051 nuclease or a nuclease domain thereof. In some embodiments, the gene editing composition comprises a fusion protein. In some embodiments, the fusion protein comprises a nuclease-inactivated Cas9 (dCas9) protein and a Clo051 nuclease or a Clo051 nuclease domain. In some embodiments, the gene editing composition further comprises a guide sequence. In some embodiments, the guide sequence comprises an RNA sequence. In some embodiments, the fusion protein comprises or consists of the amino acid sequence:

(SEQ ID NO: 17013)
MAPKKKRKVEGIKSNISLLKDELRGQISHISHEYLSLIDLAFDSKQNRLF
EMKVLELLVNEYGFKGRHLGGSRKPDGIVYSTTLEDNFGIIVDTKAYSEG
YSLPISQADEMERYVRENSNRDEEVNPNKWWENFSEEVKKYYFVFISGSF
KGKFEEQLRRLSMTTGVNGSAVNVVNLLLGAEKIRSGEMTIEELERAMFN
NSEFILKYGGGGSDKKYSIGLAIGTNSVGWAVITDEYKVPSKKEKVLGNT
DRHSIKKNLIGALLFDSGETAEATRLKRTARRRYTRRKNRICYLQEIFSN
EMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLF
GNLIALSLGLIPNEKSNEDLAEDAKLQLSKDTYDDDLDNLLAQIGDQYAD
LFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASA
QSFIERMTNEDKNLPNEKVLPKHSLLYEYFTVYNELTKVKYVTEGMRKPA
FLSGEQKKAIVDLLEKTNRKVIVKQLKEDYFKKIECEDSVEISGVEDRFN
ASLGTYHDLLKIIKDKDFLDNEENEDILEDIVLILTLFEDREMIEERLKT
YAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGIL
QTVKVVDELVKVMGRHKPENIVIEMARENQTTQKGQKNSRERMKRIEEGI
KELGSQILKEHPVENTQLQNEKLYLYYLQNGRDMYVDQELDINRLSDYDV
DAIVPQSFLKDDSIDNKVLTRSDKNRGKSDNVPSEEVVKKMKNYWRQLLN
AKLITQRKEDNLIKAERGGLSELDKAGFIKRQLVETRQITKHVAQILDSR
MNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYL
ASHYEKLKGSPEDNEQKQLFVEQHKHYLDEIIEQISEFSKRVILADANLD
KVLSAYNKHRDKPIREQAENIIHLFTLTNLGAPAAFKYFDTTIDRKRYTS
TKEVLDATLIHQSITGLYETRIDLSQLGGDGSPKKKRKVSS

or a nucleic acid comprising or consisting of the sequence:

```
(SEQ ID NO: 17014)
      1 atggcaccaa agaagaaaag aaaagtggag ggcatcaagt caaacatcag cctgctgaaa
    61 gacgaactgc ggggacagat tagtcacatc agtcacgagt acctgtcact gattgatctg
  121 gccttcgaca gcaagcagaa tagactgttt gagatgaaag tgctggaact gctggtcaac
  181 gagtatggct tcaagggcag acatctgggc gggtctagga aacctgacgg catcgtgtac
  241 agtaccacac tggaagacaa cttcggaatc attgtcgata ccaaggctta ttccgagggc
  301 tactctctgc caattagtca ggcagatgag atggaaaggt acgtgcgcga aaactcaaat
  361 agggacgagg aagtcaaccc caataagtgg tgggagaatt tcagcgagga agtgaagaaa
  421 tactacttcg tctttatctc aggcagcttc aaagggaagt ttgaggaaca gctgcggaga
  481 ctgtccatga ctaccggggt gaacggatct gctgtcaacg tggtcaatct gctgctgggc
  541 gcagaaaaga tcaggtccgg ggagatgaca attgaggaac tggaacgcgc catgttcaac
  601 aattctgagt ttatcctgaa gtatggaggc gggggaagcg ataagaaata ctccatcgga
  661 ctggccattg gcaccaattc cgtgggctgg gctgtcatca cagacgagta caaggtgcca
  721 agcaagaagt tcaaggtcct ggggaacacc gatcgccaca gtatcaagaa aaatctgatt
  781 ggagccctgc tgttcgactc aggcgagact gctgaagcaa cccgactgaa gcggactgct
  841 aggcgccgat atacccggag aaaaaatcgg atctgctacc tgcaggaaat tttcagcaac
  901 gagatggcca aggtggacga tagtttcttt caccgcctgg aggaatcatt cctggtggag
  961 gaagataaga aacacgagcg gcatcccatc tttggcaaca ttgtggacga agtcgcttat
1021 cacgagaagt accctactat ctatcatctg aggaagaaac tggtggactc caccgataag
1081 gcagacctgc gcctgatcta tctggccctg gctcacatga tcaagttccg ggggcatttt
1141 ctgatcgagg gagatctgaa ccctgacaat tctgatgtgg acaagctgtt catccagctg
1201 gtccagacat acaatcagct gtttgaggaa aacccaatta atgcctcagg cgtggacgca
1261 aaggccatcc tgagcgccag actgtccaaa tctaggcgcc tggaaaacct gatcgctcag
1321 ctgccaggag agaagaaaaa cggcctgttt gggaatctga ttgcactgtc cctgggcctg
1381 acacccaact tcaagtctaa ttttgatctg gccgaggacg ctaagctgca gctgtccaaa
1441 gacacttatg acgatgacct ggataacctg ctggctcaga tcggcgatca gtacgcagac
1501 ctgttcctgg ccgctaagaa tctgagtgac gccatcctgc tgtcagatat tctgcgcgtg
1561 aacacagaga ttactaaggc cccactgagt gcttcaatga tcaaaagata tgacgagcac
1621 catcaggatc tgaccctgct gaaggctctg gtgaggcagc agctgcccga gaaatacaag
1681 gaaatcttct ttgatcagag caagaatgga tacgccggct atattgacgg cggggcttcc
1741 caggaggagt tctacaagtt catcaagccc attctggaaa agatggacgg caccgaggaa
1801 ctgctggtga agctgaatcg ggaggacctg ctgagaaaac agaggacatt tgataacgga
1861 agcatccctc accagattca tctgggcgaa ctgcacgcca tcctgcgacg gcaggaggac
1921 ttctacccat ttctgaagga taaccgcgag aaaatcgaaa agatcctgac cttcagaatc
1981 ccctactatg tggggcctct ggcacgggga aatagtagat ttgcctggat gacaagaaag
2041 tcagaggaaa ctatcacccc ctggaacttc gaggaagtgg tcgataaagg cgctagcgca
2101 cagtccttca ttgaaaggat gacaaatttt gacaagaacc tgccaaatga gaaggtgctg
2161 cccaaacaca gcctgctgta cgaatatttc acagtgtata acgagctgac taaagtgaag
2221 tacgtcaccg aagggatgcg caagcccgca ttcctgtccg gagagcagaa gaaagccatc
2281 gtggacctgc tgtttaagac aaatcggaaa gtgactgtca aacagctgaa ggaagactat
2341 ttcaagaaaa ttgagtgttt cgattcagtg gaaatcagcg gcgtcgagga caggtttaac
2401 gcctccctgg ggacctacca cgatctgctg aagatcatca aggataagga cttcctggac
2461 aacgaggaaa atgaggacat cctggaggac attgtgctga cactgactct gtttgaggat
2521 cgcgaaatga tcgaggaacg actgaagact tatgcccatc tgttcgatga caaagtgatg
2581 aagcagctga aaagaaggcg ctacaccgga tggggacgcc tgagccgaaa actgatcaat
2641 gggattagag acaagcagag cggaaaaact atcctggact ttctgaagtc cgatggcttc
2701 gccaacagga acttcatgca gctgattcac gatgactctc tgaccttcaa ggaggacatc
2761 cagaaagcac aggtgtctgg ccagggggac agtctgcacg agcatatcgc aaacctggcc
2821 ggcagccccg ccatcaagaa agggattctg cagaccgtga aggtggtgga cgaactggtc
2881 aaggtcatgg gacgacacaa acctgagaac atcgtgattg agatggcccg cgaaaatcag
2941 acaactcaga agggccagaa aaacagtcga gaacggatga agagaatcga ggaaggcatc
3001 aaggagctgg ggtcacagat cctgaaggag catcctgtgg aaaacactca gctgcagaat
3061 gagaaactgt atctgtacta tctgcagaat ggacgggata tgtacgtgga ccaggagctg
3121 gatattaaca gactgagtga ttatgacgtg gatgccatcg tccctcagag cttcctgaag
3181 gatgactcca ttgacaacaa ggtgctgacc aggtccgaca agaaccgcgg caaatcagat
3241 aatgtgccaa gcgaggaagt ggtcaagaaa atgaagaact actggaggca gctgctgaat
3301 gccaagctga tcacacagcg gaaatttgat aacctgacta aggcagaaag aggaggcctg
3361 tctgagctgg acaaggccgg cttcatcaag cggcagctgg tggagacaag acagatcact
3421 aagcacgtcg ctcagattct ggatagcaga atgaacacaa agtacgatga aaacgacaag
3481 ctgatcaggg aggtgaaagt cattactctg aaatccaagc tggtgtctga ctttagaaag
3541 gatttccagt tttataaagt cagggagatc aacaactacc accatgctca tgacgcatac
3601 ctgaacgcag tggtcgggac cgccctgatt aagaaatacc ccaagctgga gtccgagttc
3661 gtgtacggag actataaagt gtacgatgtc cggaagatga tcgccaaatc tgagcaggaa
3721 attggcaagg ccaccgctaa gtatttcttt tacagtaaca tcatgaattt ctttaagacc
3781 gaaatcacac tggcaaatgg ggagatcaga aaaaggcctc tgattgagac caacggggag
3841 acaggagaaa tcgtgtggga caagggaagg gattttgcta ccgtgcgcaa agtcctgtcc
3901 atgccccaag tgaatattgt caagaaaact gaagtgcaga ccgggggatt ctctaaggag
3961 agtattctgc ctaagcgaaa ctctgataaa ctgatcgccc ggaagaaaga ctgggacccc
4021 aagaagtatg gcgggttcga ctctccaaca gtggcttaca gtgtcctggt ggtcgcaaag
4081 gtggaaaagg ggaagtccaa gaaactgaag tctgtcaaag agctgctggg aatcactatt
4141 atggaacgca gctccttcga gaagaatcct atcgattttc tggaagccaa gggctataaa
4201 gaggtgaaga aagacctgat cattaagctg ccaaaatact cactgtttga gctggaaaac
4261 ggacgaaagc gaatgctggc aagcgccgga gaactgcaga agggcaatga gctggccctg
4321 ccctccaaat acgtgaactt cctgtatctg gctagccact acgagaaact gaaggggtcc
4381 cctgaggata acgaacagaa gcagctgttt gtggagcagc acaaacatta tctggacgag
4441 atcattgaac agatttcaga gttcagcaag agagtgatcc tggctgacgc aaatctggat
4501 aaagtcctga gcgcatacaa caagcaccga gacaaaccaa tccgggagca ggccgaaaat
4561 atcattcatc tgttcaccct gacaaacctg ggcgcccctg cagccttcaa gtattttgac
4621 accacaatcg atcggaagag atacacttct accaaagagg tgctggatgc taccctgatc
4681 caccagagta ttaccggcct gtatgagaca cgcatcgacc tgtcacagct gggaggcgat
4741 gggagcccca agaaaaagcg gaaggtgtct agttaa.
```

In some embodiments, the fusion protein comprises or consists of the amino acid sequence:

```
((SEQ ID NO: 17058)
      1 MPKKKRKVEG IKSNISLLKD ELRGQISHIS HEYLSLIDLA FDSKQNRLFE MKVLELLVNE
    61 YGFKGRHLGG SRKPDGIVYS TTLEDNFGII VDTKAYSEGY SLPISQADEM ERYVRENSNR
  121 DEEVNPNKWW ENFSEEVKKY YFVFISGSFK GKFEEQLRRL SMTTGVNGSA VNVVNLLLGA
  181 EKIRSGEMTI EELERAMFNN SEFILKYGGG GSDKKYSIGL AIGTNSVGWA VITDEYKVPS
  241 KKFKVLGNTD RHSIKKNLIG ALLFDSGETA EATRLKRTAR RRYTRRKNRI CYLQEIFSNE
  301 MAKVDDSFFH RLEESFLVEE DKKHERHPIF GNIVDEVAYH EKYPTIYHLR KKLVDSTDKA
  361 DLRLIYLALA HMIKFRGHFL IEGDLNPDNS DVDKLFIQLV QTYNQLFEEN PINASGVDAK
  421 AILSARLSKS RRLENLIAQL PGEKKNGLFG NLIALSLGLT PNFKSNFDLA EDAKLQLSKD
  481 TYDDDLDNLL AQIGDQYADL FLAAKNLSDA ILLSDILRVN TEITKAPLSA SMIKRYDEHH
  541 QDLTLLKALV RQQLPEKYKE IFFDQSKNGY AGYIDGGASQ EEFYKFIKPI LEKMDGTEEL
  601 LVKLNREDLL RKQRTFDNGS IPHQIHLGEL HAILRRQEDF YPFLKDNREK IEKILTFRIP
  661 YYVGPLARGN SRFAWMTRKS EETITPWNFE EVVDKGASAQ SFIERMTNFD KNLPNEKVLP
  721 KHSLLYEYFT VYNELTKVKY VTEGMRKPAF LSGEQKKAIV DLLFKTNRKV TVKQLKEDYF
  781 KKIECFDSVE ISGVEDRFNA SLGTYHDLLK IIKDKDFLDN EENEDILEDI VLTLTLFEDR
  841 EMIEERLKTY AHLFDDKVMK QLKRRRYTGW GRLSRKLING IRDKQSGKTI LDFLKSDGFA
  901 NRNFMQLIHD DSLTFKEDIQ KAQVSGQGDS LHEHIANLAG SPAIKKGILQ TVKVVDELVK
  961 VMGRHKPENI VIEMARENQT TQKGQKNSRE RMKRIEEGIK ELGSQILKEH PVENTQLQNE
1021 KLYLYYLQNG RDMYVDQELD INRLSDYDVD AIVPQSFLKD DSIDNKVLTR SDKNRGKSDN
1081 VPSEEVVKKM KNYWRQLLNA KLITQRKFDN LTKAERGGLS ELDKAGFIKR QLVETRQITK
1141 HVAQILDSRM NTKYDENDKL IREVKVITLK SKLVSDFRKD FQFYKVREIN NYHHAHDAYL
1201 NAVVGTALIK KYPKLESEFV YGDYKVYDVR KMIAKSEQEI GKATAKYFFY SNIMNFFKTE
1261 ITLANGEIRK RPLIETNGET GEIVWDKGRD FATVRKVLSM PQVNIVKKTE VQTGGFSKES
1321 ILPKRNSDKL IARKKDWDPK KYGGFDSPTV AYSVLVVAKV EKGKSKKLKS VKELLGITIM
1381 ERSSFEKNPI DFLEAKGYKE VKKDLIIKLP KYSLFELENG RKRMLASAGE LQKGNELALP
1441 SKYVNFLYLA SHYEKLKGSP EDNEQKQLFV EQHKHYLDEI IEQISEFSKR VILADANLDK
1501 VLSAYNKHRD KPIREQAENI IHLFTLINLG APAAFKYFDT TIDRKRYTST KEVLDATLIH
1561 QSITGLYETR IDLSQLGGDG SPKKKRKV
```

or a nucleic acid comprising or consisting of the sequence:

```
(SEQ ID NO: 17059)
      1 atgcctaaga agaagcggaa ggtggaaggc atcaaaagca acatctccct cctgaaagac
    61 gaactccggg ggcagattag ccacattagt cacgaatacc tctccctcat cgacctggct
  121 ttcgatagca agcagaacag gctctttgag atgaaagtgc tggaactgct cgtcaatgag
  181 tacgggttca agggtcgaca cctcggcgga tctaggaaac cagacggcat cgtgtatagt
  241 accacactgg aagacaactt tgggatcatt gtggatacca aggcatactc tgagggttat
  301 agtctgccca tttcacaggc cgacgagatg gaacggtacg tgcgcgagaa ctcaaataga
  361 gatgaggaag tcaaccctaa caagtggtgg gagaacttct ctgaggaagt gaagaaatac
  421 tacttcgtct ttatcagcgg gtccttcaag ggtaaatttg aggaacagct caggagactg
  481 agcatgacta ccggcgtgaa tggcagcgcc gtcaacgtgg tcaatctgct cctgggcgct
  541 gaaaagattc ggagcggaga gatgaccatc gaagagctgg agagggcaat gtttaataat
  601 agcgagttta tcctgaaata cggtggcggt ggatccgata aaaagtattc tattggttta
  661 gccatcggca ctaattccgt tggatgggct gtcataaccg atgaatacaa agtaccttca
  721 aagaaattta aggtgttggg gaacacagac cgtcattcga ttaaaaagaa tcttatcggt
  781 gccctcctat tcgatagtgg cgaaacggca gaggcgactc gcctgaaacg aaccgctcgg
  841 agaaggtata cacgtcgcaa gaaccgaata tgttacttac aagaaatttt tagcaatgag
  901 atggccaaag ttgacgattc tttctttcac cgtttggaag agtccttcct tgtcgaagag
  961 gacaagaaac atgaacggca ccccatcttt ggaaacatag tagatgaggt ggcatatcat
1021 gaaaagtacc caacgattta tcacctcaga aaaaagctag ttgactcaac tgataaagcg
1081 gacctgaggt taatctactt ggctcttgcc catatgataa agttccgtgg gcactttctc
1141 attgagggtg atctaaatcc ggacaactcg gatgtcgaca aactgttcat ccagttagta
1201 caaacctata atcagttgtt tgaagagaac cctataaatg caagtggcgt ggatgcgaag
1261 gctattctta gcgcccgcct ctctaaatcc cgacggctag aaaacctgat cgcacaatta
1321 cccggagaga agaaaaatgg gttgttcggt aaccttatag cgctctcact aggcctgaca
1381 ccaaatttta agtcgaactt cgacttagct gaagatgcca aattgcagct tagtaaggac
1441 acgtacgatg acgatctcga caatctactg gcacaaattg gagatcagta tgcggactta
1501 tttttggctg ccaaaaacct tagcgatgca atcctcctat ctgacatact gagagttaat
1561 actgagatta ccaaggcgcc gttatccgct tcaatgatca aaaggtacga tgaacatcac
1621 caagacttga cacttctcaa ggccctagtc cgtcagcaac tgcctgagaa atataaggaa
1681 atattctttg atcagtcgaa aaacgggtac gcaggttata ttgacggcgg agcgagtcaa
1741 gaggaattct acaagtttat caaacccata ttagagaaga tggatgggac ggaagagttg
1801 cttgtaaaac tcaatcgcga agatctactg cgaaagcagc ggactttcga caacggtagc
1861 attccacatc aaatccactt aggcgaattg catgctatac ttagaaggca ggaggatttt
1921 tatccgttcc tcaaagacaa tcgtgaaaag attgagaaaa tcctaacctt tcgcatacct
1981 tactatgtgg gacccctggc ccgagggaac tctcggttcg catggatgac aagaaagtcc
2041 gaagaaacga ttactccatg gaattttgag gaagttgtcg ataaaggtgc gtcagctcaa
2101 tcgttcatcg agaggatgac caactttgac aagaatttac cgaacgaaaa agtattgcct
2161 aagcacagtt tactttacga gtatttcaca gtgtacaatg aactcacgaa agttaagtat
2221 gtcactgagg gcatgcgtaa acccgccttt ctaagcggag aacagaagaa agcaatagta
2281 gatctgttat tcaagaccaa ccgcaaagtg acagttaagc aattgaaaga ggactacttt
2341 aagaaaattg aatgcttcga ttctgtcgag atctccgggg tagaagatcg atttaatgcg
2401 tcacttggta cgtatcatga cctcctaaag ataattaaag ataaggactt cctggataac
2461 gaagagaatg aagatatctt agaagatata gtgttgactc ttaccctctt tgaagatcgg
2521 gaaatgattg aggaaagact aaaaacatac gctcacctgt tcgacgataa ggttatgaaa
2581 cagttaaaga ggcgtcgcta tacgggctgg ggacgattgt cgcggaaact tatcaacggg
2641 ataagagaca agcaaagtgg taaaactatt ctcgattttc taaagagcga cggcttcgcc
2701 aataggaact ttatgcagct gatccatgat gactctttaa ccttcaaaga ggatatacaa
2761 aaggcacagg tttccggaca aggggactca ttgcacgaac atattgcgaa tcttgctggt
2821 tcgccagcca tcaaaaaggg catactccag acagtcaaag tagtggatga gctagttaag
2881 gtcatgggac gtcacaaacc ggaaaacatt gtaatcgaga tggcacgcga aaatcaaacg
2941 actcagaagg ggcaaaaaaa cagtcgagag cggatgaaga gaatagaaga gggtattaaa
3001 gaactgggca gccagatctt aaaggagcat cctgtggaaa atacccaatt gcagaacgag
3061 aaactttacc tctattacct acaaaatgga agggacatgt atgttgatca ggaactggac
3121 ataaaccgtt tatctgatta cgacgtcgat gccattgtac cccaatcctt tttgaaggac
3181 gattcaatcg acaataaagt gcttacacgc tcggataaga accgagggaa aagtgacaat
3241 gttccaagcg aggaagtcgt aaagaaaatg aagaactatt ggcggcagct cctaaatgcg
3301 aaactgataa cgcaaagaaa gttcgataac ttaactaaag ctgagagggg tggcttgtct
3361 gaacttgaca aggccggatt tattaaacgt cagctcgtgg aaacccgcca aatcacaaag
3421 catgttgcac agatactaga ttcccgaatg aatacgaaat acgacgagaa cgataagctg
3481 attcgggaag tcaaagtaat cactttaaag tcaaaattgg tgtcggactt cagaaaggat
3541 tttcaattct ataaagttag ggagataaat aactaccacc atgcgcacga cgcttatctt
3601 aatgccgtcg tagggaccgc actcattaag aaatacccga agctagaaag tgagtttgtg
3661 tatggtgatt acaaagttta tgacgtccgt aagatgatcg cgaaaagcga acaggagata
3721 ggcaaggcta cagccaaata cttcttttat tctaacatta tgaatttctt taagacggaa
3781 atcactctgg caaacggaga gatacgcaaa cgacctttaa ttgaaaccaa tggggagaca
3841 ggtgaaatcg tatgggataa gggccgggac ttcgcgacgg tgagaaaagt tttgtccatg
3901 ccccaagtca acatagtaaa gaaaactgag gtgcagaccg gagggttttc aaaggaatcg
3961 attcttccaa aaaggaatag tgataagctc atcgctcgta aaaaggactg ggacccgaaa
4021 aagtacggtg gcttcgatag ccctacagtt gcctattctg tcctagtagt ggcaaaagtt
4081 gagaagggaa aatccaagaa actgaagtca gtcaaagaat tattggggat aacgattatg
4141 gagcgctcgt cttttgaaaa gaaccccatc gacttccttg aggcgaaagg ttacaaggaa
4201 gtaaaaaagg atctcataat taaactacca aagtatagtc tgtttgagtt agaaaatggc
4261 cgaaaacgga tgttggctag cgccggagag cttcaaaagg ggaacgaact cgcactaccg
4321 tctaaatacg tgaatttcct gtatttagcg tcccattacg agaagttgaa aggttcacct
4381 gaagataacg aacagaagca actttttgtt gagcagcaca aacattatct cgacgaaatc
4441 atagagcaaa tttcggaatt cagtaagaga gtcatcctag ctgatgccaa tctggacaaa
4501 gtattaagcg catacaacaa gcacagggat aaacccatac gtgagcaggc ggaaaatatt
4561 atccatttgt ttactcttac caacctcggc gctccagccg cattcaagta ttttgacaca
4621 acgatagatc gcaaacgata cacttctacc aaggaggtgc tagacgcgac actgattcac
4681 caatccatca cgggattata tgaaactcgg atagatttgt cacagcttgg gggtgacgga
4741 tcccccaaga agaagaggaa agtctga.
```

In some embodiments of the cells, unmodified cells and modified cells of the disclosure, a nanotransposon comprises the gene editing composition comprising a guide sequence and a sequence encoding a fusion protein comprising a sequence encoding an inactivated Cas9 (dCas9) and a sequence encoding a Clo051 nuclease or a nuclease domain thereof.

In some embodiments of the cells, unmodified cells and modified cells of the disclosure, the cell expresses the gene editing composition transiently.

In some embodiments of the cells, unmodified cells and modified cells of the disclosure, the cell is a T cell and the guide RNA comprises a sequence complementary to a target sequence encoding an endogenous TCR. In some embodiments, the guide RNA comprises a sequence complementary to a target sequence encoding a B2M polypeptide.

In some embodiments of the cells, unmodified cells and modified cells of the disclosure, the guide RNA comprises a sequence complementary to a target sequence within a safe harbor site of a genomic DNA sequence.

In some embodiments of the cells, unmodified cells and modified cells of the disclosure, the Clo051 nuclease or a nuclease domain thereof induces a single or double strand break in a target sequence. In some embodiments, a donor sequence, a donor plasmid, or a donor nanotransposon intra-ITR sequence integrated at a position of single or double strand break and/or at a position of cellular repair within a target sequence.

The disclosure provides a composition comprising a modified cell according to the disclosure. In some embodiments, the composition further comprises a pharmaceutically-acceptable carrier.

The disclosure provides a composition comprising a plurality of modified cells according to the disclosure. In some embodiments, the composition further comprises a pharmaceutically-acceptable carrier.

The disclosure provides a composition of the disclosure for use in the treatment of a disease or disorder.

The disclosure provides the use of a composition of the disclosure for the treatment of a disease or disorder.

The disclosure provides a method of treating a disease or disorder comprising administering to a subject in need thereof a therapeutically-effective amount of a composition of the disclosure. In some embodiments, the subject does not develop graft vs. host (GvH) and/or host vs. graft (HvG) following administration of the composition. In some embodiments, the administration is systemic. In some embodiments, the composition is administered by an intravenous route. In some embodiments, the composition is administered by an intravenous injection or an intravenous infusion.

The disclosure provides a method of treating a disease or disorder comprising administering to a subject in need thereof a therapeutically-effective amount of a composition of the disclosure. In some embodiments, the subject does not develop graft vs. host (GvH) and/or host vs. graft (HvG) following administration of the composition. In some embodiments, the administration is local. In some embodiments, the composition is administered by an intra-tumoral route, an intraspinal route, an intracerebroventricular route, an intraocular route or an intraosseous route. In some embodiments, the composition is administered by an intra-tumoral injection or infusion, an intraspinal injection or infusion, an intracerebroventricular injection or infusion, an intraocular injection or infusion or an intraosseous injection or infusion.

In some embodiments of the methods of treating a disease or disorder of the disclosure, the therapeutically effective dose is a single dose and wherein the allogeneic cells of the composition engraft and/or persist for a sufficient time to treat the disease or disorder. In some embodiments, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously.

In some embodiments of the methods of treating a disease or disorder of the disclosure, the therapeutically effective dose is a single dose and wherein the autologous cells of the composition engraft and/or persist for a sufficient time to treat the disease or disorder. In some embodiments, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously.

### Vectors and Host Cells

The disclosure also relates to vectors that include isolated nucleic acid molecules of the disclosure, host cells that are genetically engineered with the recombinant vectors, and the production of at least one Centyrin or CARTyrin by recombinant techniques, as is well known in the art. See, e.g., Sambrook, et al., supra; Ausubel, et al., supra, each entirely incorporated herein by reference.

For example, the PB-EF1a vector may be used. The vector comprises the following nucleotide sequence:

(SEQ ID NO: 18051)
tgtacatagattaaccctagaaagataatcatattgtgacgtacgttaaagataatcatgcgtaaaattgacgcatgtgttttatcggtctgt
atatcgaggtttatttattaatttgaatagatattaagttttattatatttacacttacatactaataataaattcaacaaacaatttatttatgtttatt
tatttattaaaaaaaaacaaaaactcaaaatttcttctataaagtaacaaaacttttatcgaatacctgcagcccgggggatgcagaggga
cagcccccccccaaagcccccagggatgtaattacgtccctcccccgctagggggcagcagcgagccgcccggggctccgctcc
ggtccggcgctccccccgcatccccgagccggcagcgtgcggggacagcccgggcacggggaaggtggcacgggatcgctttc
ctctgaacgcttctcgctgctattgagcctgcagacacctggggggatacggggaaaagttgactgtgcctttcgatcgaaccatgga
cagttagctttgcaaagatggataaagttttaaacagagaggaatctagcagctaatggaccttctaggtcttgaaaggagtgggaattg
gctccggtgcccgtcagtgggcagagcgcacatcgcccacagtccccgagaagttggggggaggggtcggcaattgaaccggtg
cctagagaaggtggcgcggggtaaactgggaaagtgatgtcgtgtactggctccgcctttttcccgagggtgggggagaaccgtata
taagtgcagtagtcgccgtgaacgttctttttcgcaacgggtttgccgccagaacacaggtaagtgccgtgtgtggttcccgcgggcct
ggcctctttacgggttatggcccttgcgtgccttgaattacttccacctggctgcagtacgtgattcttgatcccgagcttcgggttggaag
tgggtgggagagttcgaggccttgcgcttaaggagccccttcgcctcgtgcttgagttgaggcctggcctgggcgctggggccgccg
cgtgcgaatctggtggcaccttcgcgcctgtctcgctgctttcgataagtctctagccatttaaaatttttgatgacctgctgcgacgcttttt
ttctggcaagatagtcttgtaaatgcgggccaagatctgcacactggtatttcggataggggccgcgggcggcgacggggcccgtg
cgtcccagcgcacatgttcggcgaggcggggcctgcgagcgcggccaccgagaatcggacgggggtagtctcaagctggccggc
ctgctctggtgcctggcctcgcgccgccgtgtatcgccccgccctgggcggcaaggctggcccggtcggcaccagttgcgtgagcg
gaaagatggccgcttcccggccctgctgcagggagctcaaaatggaggacgcggcgctcgggagagcgggcgggtgagtcaccc
acacaaaggaaaagggcctttccgtcctcagccgtcgcttcatgtgactccacggagtaccgggcgccgtccaggcacctcgattagt
tctcgagcttttggagtacgtcgtctttaggttggggggaggggttttatgcgatggagtttccccacactgagtgggtggagactgaag
ttaggccagcttggcacttgatgtaattctccttggaatttgccctattgagtttggatcttggttcattctcaagcctcagacagtggttcaa
agtttttttcttccatttcaggtgtcgtgagaattctaatacgactcactatagggtgtgctgtctcatcattttggcaaagattggccaccaa
gcttgtcctgcaggagggtcgacgcctctagacgggcggccgctccggatccacgggtaccgatcacatatgcctttaattaaacact
agttctatagtgtcacctaaattccctttagtgagggttaatggccgtaggccgccagaattgggtccagacatgataagatacattgatg
agtttggacaaaccacaactagaatgcagtgaaaaaaatgctttatttgtgaaatttgtgatgctattgctttatttgtaaccattataagctg
caataaacaagttaacaacaacaattgcattcatatatgtacaggttcagggggaggtgtgggaggattacggactctaggacctgcg
catgcgcttggcgtaatcatggtcatagctgtttcctgttttccccgtatccccccaggtgtctgcaggctcaaagagcagcgagaagcg
ttcagaggaaagcgatcccgtgccaccttccccgtgcccgggctgtccccgcacgctgccggctcggggatgcggggggagcgcc
ggaccggagcggagccccgggcggctcgctgctgccccctagcgggggagggacgtaattacatccctgggggctttggggggg
ggctgtccctctcaccgcggtggagctccagcttttgttcgaattggggccccccctcgagggtatcgatgatatctataacaagaaaat
atatatataataagttatcacgtaagtagaacatgaaataacaatataattatcgtatgagttaaatcttaaaagtcacgtaaaagataatcat
gcgtcattttgactcacgcggtcgttatagttcaaaatcagtgacacttaccgcattgacaagcacgcctcacgggagctccaagcggc
gactgagatgtcctaaatgcacagcgacggattcgcgctatttagaaagagagagcaatatttcaagaatgcatgcgtcaattttacgca
gactatctttctagggttaatctagctagccttaagggcgcctattgcgttgcgctcactgcccgctttccagtcgggaaacctgtcgtgc
cagctgcattaatgaatcggccaacgcgcggggagaggcggtagcgtattgggcgctcttccgcttcctcgctcactgactcgctgc
gctcggtcgttcggctgcggcgagcggtatcagctcactcaaaggcggtaatacggttatccacagaatcaggggataacgcaggaa
agaacatgaccaaaatcccttaacgtgagttttcgttccactgagcgtcagaccccgtagaaaagatcaaaggatcttcttgagatccttt
ttttctgcgcgtaatctgctgcttgcaaacaaaaaaaccaccgctaccagcggtggtttgtttgccggatcaagagctaccaactctttttc
cgaaggtaactggcttcagcagagcgcagataccaaatactgttcttctagtgtagccgtagttaggccaccacttcaagaactctgtag
caccgcctacatacctcgctctgctaatcctgttaccagtggctgctgccagtggcgataagtcgtgtcttaccgggttggactcaagac
gatagttaccggataaggcgcagcggtcgggctgaacggggggttcgtgcacacagcccagcttggagcgaacgacctacaccga
actgagatacctacagcgtgagctatgagaaagcgccacgcttcccgaagggagaaaggcggacaggtatccggtaagcggcagg
gtcggaacaggagagcgcacgagggagcttccagggggaaacgcctggtatctttatagtcctgtcgggtttcgccacctctgacttg
agcgtcgatttttgtgatgctcgtcaggggggcggagcctatggaaaaacgccagcaacgcggcctttttacggttcctggccttagct
ggccattgctcacatgagattatcaaaaaggatcttcacctagatccttttaaattaaaaatgaagattaaatcaatctaaagtatatatga
gtaaacttggictgacagtcagaagaactcgtcaagaaggcgatagaaggcgatgcgctgcgaatcgggagcggcgataccgtaaa
gcacgaggaagcggtcagcccattcgccgccaagctcttcagcaatatcacgggtagccaacgctatgtcctgatagcggtccgcca
cacccagccggccacagtcgatgaatccagaaaagcggccattttccaccatgatattcggcaagcaggcatcgccatgggtcacga
cgagatcctcgccgtcgggcatgctcgccttgagcctggcgaacagttcggctggcgcgagcccctgatgctcttcgtccagatcatc
ctgatcgacaagaccggcttccatccgagtacgtgctcgctcgatgcgatgtttcgcttggtggtcgaatgggcaggtagccggatca
agcgtatgcagccgccgcattgcatcagccatgatggatactttctcggcaggagcaaggtgagatgacaggagatcctgccccggc
acttcgcccaatagcagccagtcccttcccgcttcagtgacaacgtcgagcacagctgcgcaaggaacgcccgtcgtggccagcca
cgatagccgcgctgcctcgtcttgcagttcattcagggcaccggacaggtcggtcttgacaaaaagaaccgggcgcccctgcgctga
cagccggaacacggcggcatcagagcagccgattgtctgttgtgcccagtcatagccgaatagcctctccacccaagcggccggag
aacctgcgtgcaatccatcttgttcaatcataatattattgaagcatttatcagggttcgtctcgtcccggtctcctcccaatgcatgtcaata
ttggccattagccatattattcattggttatatagcataaatcaatattggctattggccattgcatacgttgtatctatatcataata

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124(8):1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, TYMS, FRANCF, RAD51C, GCS, MDR1, ALDH1, NKX2.2, or any combination thereof.

At least one Centyrin or CARTyrin of the disclosure can be expressed in a modified form, such as a fusion protein, and can include not only secretion signals, but also additional heterologous functional regions. For instance, a region of additional amino acids, particularly charged amino acids, can be added to the N-terminus of a CARTyrin to improve stability and persistence in the host cell, during purification, or during subsequent handling and storage. Also, peptide moieties can be added to a CARTyrin of the disclosure to facilitate purification. Such regions can be removed prior to final preparation of a CARTyrin or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein of the disclosure. Alternatively, nucleic acids of the disclosure can be expressed in a host cell by turning on (by manipulation) in a host cell that contains endogenous DNA encoding a Centyrin or CARTyrin of the disclosure. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the CARTyrins, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, 5P2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or an SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

### Purification of a CARTyrin

A Centyrin or CARTyrin can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Centyrins or CARTyrins of the disclosure include purified products, products of chemical synthetic procedures, and products produced by recombinant techniques from a prokaryotic or eukaryotic host, including, for example, E. coli, yeast, higher plant, insect and mammalian cells. Depending upon the host employed in a recombinant production procedure, the CARTyrin of the disclosure can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

### Amino Acid Codes

The amino acids that make up CARtyrins of the disclosure are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994). A CARTyrin of the disclosure can include one or more amino acid substitutions, deletions or additions, from spontaneous or mutations and/or human manipulation, as specified herein. Amino acids in a CARTyrin of the disclosure that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one neutralizing activity. Sites that are critical for CARTyrin binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

As those of skill will appreciate, the invention includes at least one biologically active CARTyrin of the disclosure. Biologically active CARTyrins have a specific activity at least 20%, 30%, or 40%, and, preferably, at least 50%, 60%, or 70%, and, most preferably, at least 80%, 90%, or 95%-99% or more of the specific activity of the native (non-synthetic), endogenous or related and known CARTyrin. Methods of assaying and quantifying measures of enzymatic activity and substrate specificity are well known to those of skill in the art.

In another aspect, the disclosure relates to Centyrins and fragments, as described herein, which are modified by the covalent attachment of an organic moiety. Such modification can produce a CARTyrin fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified CARTyrin and fragments of the disclosure can comprise one or more organic moieties that are covalently bonded, directly or indirectly, to the antibody. Each organic moiety that is bonded to a CARTyrin or fragment thereof of the disclosure can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, a Centyrin or CARTyrin modified by the covalent attachment of polylysine is encompassed by the disclosure. Hydrophilic polymers suitable for modifying Centyrins or CARTyrins of the disclosure can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the CARTyrin of the disclosure has a molecular weight of about 800 to about 150,000 Daltons as a separate molecular entity. For example, PEG5000 and PEG 20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying CARTyrins of the disclosure can be saturated or can contain one or more units of unsaturation. Fatty acids that are suitable for modifying CARtyrins of the disclosure include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-Δ9-octadecanoate (C18, oleate), all cis-Δ5,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified CARTyrins and fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, —(CH2)3-, —NH—(CH2)6-NH—, —(CH2)2-NH— and —CH2-O—CH2-CH2-O—CH2-CH2-O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified CARTyrins and fragments of the disclosure can be produced by reacting CARTyrin proteins or fragments with a modifying agent. For example, the organic moieties can be bonded to the CARTyrin protein in a non-site specific manner by employing an amine-reactive modifying agent, for example, an NHS ester of PEG. Modified CARTyrin proteins and fragments comprising an organic moiety that is bonded to specific sites of a CARTyrin of the disclosure can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

### CARTyrin Compositions Comprising Further Therapeutically Active Ingredients

Centyrin or CARTyrin compounds, compositions or combinations of the present disclosure can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of the Centyrin or CARTyrin, fragment or variant composition as well known in the art or as described herein.

Pharmaceutical excipients and additives useful in the present composition include, but are not limited to, proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Exemplary protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

CARTyrin compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts, such as citrate.

Additionally, CARTyrin compositions of the invention can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the Centyrin or CARTyrin, portion or variant compositions according to the invention are known in the art, e.g., as listed in “Remington: The Science & Practice of Pharmacy”, 19th ed., Williams & Williams, (1995), and in the “Physician's Desk Reference”, 52nd ed., Medical Economics, Montvale, N.J. (1998), the disclosures of which are entirely incorporated herein by reference. Preferred carrier or excipient materials are carbohydrates (e.g., saccharides and alditols) and buffers (e.g., citrate) or polymeric agents. An exemplary carrier molecule is the mucopolysaccharide, hyaluronic acid, which may be useful for intraarticular delivery.

T Cell Isolation from a Leukapheresis Product

A leukapheresis product or blood may be collected from a subject at clinical site using a closed system and standard methods (e.g., a COBE Spectra Apheresis System). Preferably, the product is collected according to standard hospital or institutional Leukapheresis procedures in standard Leukapheresis collection bags. For example, in preferred embodiments of the methods of the disclosure, no additional anticoagulants or blood additives (heparin, etc.) are included beyond those normally used during leukapheresis.

Alternatively, white blood cells (WBC)/Peripheral Blood Mononuclear Cells (PBMC) (using Biosafe Sepax 2 (Closed/Automated)) or T cells (using CliniMACS® Prodigy (Closed/Automated)) may be isolated directly from whole blood. However, in certain subjects (e.g. those diagnosed and/or treated for cancer), the WBC/PBMC yield may be significantly lower when isolated from whole blood than when isolated by leukapheresis.

Either the leukapheresis procedure and/or the direct cell isolation procedure may be used for any subject of the disclosure.

The leukapheresis product, blood, WBC/PBMC composition and/or T-cell composition should be packed in insulated containers and should be kept at controlled room temperature (+19° C. to +25° C.) according to standard hospital of institutional blood collection procedures approved for use with the clinical protocol. The leukapheresis product, blood, WBC/PBMC composition and/or T-cell composition should not be refrigerated.

The cell concentration leukapheresis product, blood, WBC/PBMC composition and/or T-cell composition should not exceed 0.2×10⁹ cells per mL during transportation. Intense mixing of the leukapheresis product, blood, WBC/PBMC composition and/or T-cell composition should be avoided.

If the leukapheresis product, blood, WBC/PBMC composition and/or T-cell composition has to be stored, e.g. overnight, it should be kept at controlled room temperature (same as above). During storage, the concentration of the leukapheresis product, blood, WBC/PBMC composition and/or T-cell composition should never exceed 0.2×10⁹ cell per mL.

Preferably, cells of the leukapheresis product, blood, WBC/PBMC composition and/or T-cell composition should be stored in autologous plasma. In certain embodiments, if the cell concentration of the leukapheresis product, blood, WBC/PBMC composition and/or T-cell composition is higher than 0.2×10⁹ cell per mL, the product should be diluted with autologous plasma.

Preferably, the leukapheresis product, blood, WBC/PBMC composition and/or T-cell composition should not be older than 24 hours when starting the labeling and separation procedure. The leukapheresis product, blood, WBC/PBMC composition and/or T-cell composition may be processed and/or prepared for cell labeling using a closed and/or automated system (e.g., CliniMACS Prodigy).

An automated system may perform additional buffy coat isolation, possibly by ficolation, and/or washing of the cellular product (e.g., the leukapheresis product, blood, WBC/PBMC composition and/or T cell composition).

A closed and/or automated system may be used to prepare and label cells for T-Cell isolation (from, for example, the leukapheresis product, blood, WBC/PBMC composition and/or T cell composition).

Although WBC/PBMCs may be nucleofected directly (which is easier and saves additional steps), the methods of the disclosure may include first isolating T cells prior to nucleofection. The easier strategy of directly nucleofecting PBMC requires selective expansion of CARTyrin+ cells that is mediated via CARTyrin signaling, which by itself is proving to be an inferior expansion method that directly reduces the in vivo efficiency of the product by rendering T cells functionally exhausted. The product may be a heterogeneous composition of CARTyrin+ cells including T cells, NK cells, NKT cells, monocytes, or any combination thereof, which increases the variability in product from patient to patient and makes dosing and CRS management more difficult. Since T cells are thought to be the primary effectors in tumor suppression and killing, T cell isolation for the manufacture of an autologous product may result in significant benefits over the other more heterogeneous composition.

T cells may be isolated directly, by enrichment of labeled cells or depletion of labeled cells in a one-way labeling procedure or, indirectly, in a two-step labeling procedure. According to certain enrichment strategies of the disclosure, T cells may be collected in a Cell Collection Bag and the non-labeled cells (non-target cells) in a Negative Fraction Bag. In contrast to an enrichment strategy of the disclosure, the non-labeled cells (target cells) are collected in a Cell Collection Bag and the labeled cells (non-target cells) are collected in a Negative Fraction Bag or in the Non-Target Cell Bag, respectively. Selection reagents may include, but are not limited to, antibody-coated beads. Antibody-coated beads may either be removed prior to a modification and/or an expansion step, or, retained on the cells prior to a modification and/or an expansion step. One or more of the following non-limiting examples of cellular markers may be used to isolate T-cells: CD3, CD4, CD8, CD25, anti-biotin, CD1c, CD3/CD19, CD3/CD56, CD14, CD19, CD34, CD45RA, CD56, CD62L, CD133, CD137, CD271, CD304, IFN-gamma, TCR alpha/beta, and/or any combination thereof. Methods for the isolation of T-cells may include one or more reagents that specifically bind and/or detectably-label one or more of the following non-limiting examples of cellular markers may be used to isolate T-cells: CD3, CD4, CD8, CD25, anti-biotin, CD1c, CD3/CD19, CD3/CD56, CD14, CD19, CD34, CD45RA, CD56, CD62L, CD133, CD137, CD271, CD304, IFN-gamma, TCR alpha/beta, and/or any combination thereof. These reagents may or may not be “Good Manufacturing Practices” (“GMP”) grade. Reagents may include, but are not limited to, Thermo DynaBeads and Miltenyi CliniMACS products. Methods of isolating T-cells of the disclosure may include multiple iterations of labeling and/or isolation steps. At any point in the methods of isolating T-cells of the disclosure, unwanted cells and/or unwanted cell types may be depleted from a T cell product composition of the disclosure by positively or negatively selecting for the unwanted cells and/or unwanted cell types. A T cell product composition of the disclosure may contain additional cell types that may express CD4, CD8, and/or another T cell marker(s).

Methods of the disclosure for nucleofection of T cells may eliminate the step of T cell isolation by, for example, a process for nucleofection of T cells in a population or composition of WBC/PBMCs that, following nucleofection, includes an isolation step or a selective expansion step via TCR signaling.

Certain cell populations may be depleted by positive or negative selection before or after T cell enrichment and/or sorting. Examples of cell compositions that may be depleted from a cell product composition may include myeloid cells, CD25+ regulatory T cells (T Regs), dendritic cells, macrophages, red blood cells, mast cells, gamma-delta T cells, natural killer (NK) cells, a Natural Killer (NK)-like cell (e.g. a Cytokine Induced Killer (CIK) cell), induced natural killer (iNK) T cells, NK T cells, B cells, or any combination thereof.

T cell product compositions of the disclosure may include CD4+ and CD8+ T-Cells. CD4+ and CD8+ T-Cells may be isolated into separate collection bags during an isolation or selection procedure. CD4+ T cells and CD8+ T cells may be further treated separately, or treated after reconstitution (combination into the same composition) at a particular ratio.

The particular ratio at which CD4+ T cells and CD8+ T cells may be reconstituted may depend upon the type and efficacy of expansion technology used, cell medium, and/or growth conditions utilized for expansion of T-cell product compositions. Examples of possible CD4+: CD8+ ratios include, but are not limited to, 50%:50%, 60%:40%, 40%:60% 75%:25% and 25%:75%.

CD8+ T cells exhibit a potent capacity for tumor cell killing, while CD4+ T cells provide many of the cytokines required to support CD8+ T cell proliferative capacity and function. Because T cells isolated from normal donors are predominantly CD4+, the T-cell product compositions are artificially adjusted in vitro with respect to the CD4+:CD8+ ratio to improve upon the ratio of CD4+ T cells to CD8+ T cells that would otherwise be present in vivo. An optimized ratio may also be used for the ex vivo expansion of the autologous T-cell product composition. In view of the artificially adjusted CD4+:CD8+ ratio of the T-cell product composition, it is important to note that the product compositions of the disclosure may be significantly different and provide significantly greater advantage than any endogenously-occurring population of T-cells.

Preferred methods for T cell isolation may include a negative selection strategy for yielding untouched pan T cell, meaning that the resultant T-cell composition includes T-cells that have not been manipulated and that contain an endogenously-occurring variety/ratio of T-cells.

Reagents that may be used for positive or negative selection include, but are not limited to, magnetic cell separation beads. Magnetic cell separation beads may or may not be removed or depleted from selected populations of CD4+ T cells, CD8+ T cells, or a mixed population of both CD4+ and CD8+ T cells before performing the next step in a T-cell isolation method of the disclosure.

T cell compositions and T cell product compositions may be prepared for cryopreservation, storage in standard T Cell Culture Medium, and/or genetic modification.

T cell compositions, T cell product compositions, unstimulated T cell compositions, resting T cell compositions or any portion thereof may be cryopreserved using a standard cryopreservation method optimized for storing and recovering human cells with high recovery, viability, phenotype, and/or functional capacity. Commercially-available cryopreservation media and/or protocols may be used. Cryopreservation methods of the disclosure may include a DMSO free cryopreservant (e.g. CryoSOfree™ DMSO-free Cryopreservation Medium) reduce freezing-related toxicity.

T cell compositions, T cell product compositions, unstimulated T cell compositions, resting T cell compositions or any portion thereof may be stored in a culture medium. T cell culture media of the disclosure may be optimized for cell storage, cell genetic modification, cell phenotype and/or cell expansion. T cell culture media of the disclosure may include one or more antibiotics. Because the inclusion of an antibiotic within a cell culture media may decrease transfection efficiency and/or cell yield following genetic modification via nucleofection, the specific antibiotics (or combinations thereof) and their respective concentration(s) may be altered for optimal transfection efficiency and/or cell yield following genetic modification via nucleofection.

T cell culture media of the disclosure may include serum, and, moreover, the serum composition and concentration may be altered for optimal cell outcomes. Human AB serum is preferred over FBS/FCS for culture of T cells because, although contemplated for use in T cell culture media of the disclosure, FBS/FCS may introduce xeno-proteins. Serum may be isolated form the blood of the subject for whom the T-cell composition in culture is intended for administration, thus, a T cell culture medium of the disclosure may comprise autologous serum. Serum-free media or serum-substitute may also be used in T-cell culture media of the disclosure. In certain embodiments of the T-cell culture media and methods of the disclosure, serum-free media or serum-substitute may provide advantages over supplementing the medium with xeno-serum, including, but not limited to, healthier cells that have greater viability, nucleofect with higher efficiency, exhibit greater viability post-nucleofection, display a more desirable cell phenotype, and/or greater/faster expansion upon addition of expansion technologies.

T cell culture media may include a commercially-available cell growth media. Exemplary commercially-available cell growth media include, but are not limited to, PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium, or any combination thereof.

T cell compositions, T cell product compositions, unstimulated T cell compositions, resting T cell compositions or any portion thereof may be prepared for genetic modification. Preparation of T cell compositions, T cell product compositions, unstimulated T cell compositions, resting T cell compositions or any portion thereof for genetic modification may include cell washing and/or resuspension in a desired nucleofection buffer. Cryopreserved T-cell compositions may be thawed and prepared for genetic modification by nucleofection. Cryopreserved cells may be thawed according to standard or known protocols. Thawing and preparation of cryopreserved cells may be optimized to yield cells that have greater viability, nucleofect with higher efficiency, exhibit greater viability post-nucleofection, display a more desirable cell phenotype, and/or greater/faster expansion upon addition of expansion technologies. For example, Grifols Albutein (25% human albumin) may be used in the thawing and/or preparation process.

### Genetic Modification of an Autologous T Cell Product Composition

T cell compositions, T cell product compositions, unstimulated T cell compositions, resting T cell compositions or any portion thereof may be genetically modified using, for example, a nucleofection strategy such as electroporation. The total number of cells to be nucleofected, the total volume of the nucleofection reaction, and the precise timing of the preparation of the sample may be optimized to yield cells that have greater viability, nucleofect with higher efficiency, exhibit greater viability post-nucleofection, display a more desirable cell phenotype, and/or greater/faster expansion upon addition of expansion technologies.

Nucleofection and/or electroporation may be accomplished using, for example, Lonza Amaxa, MaxCyte PulseAgile, Harvard Apparatus BTX, and/or Invitrogen Neon. Non-metal electrode systems, including, but not limited to, plastic polymer electrodes, may be preferred for nucleofection.

Prior to genetic modification by nucleofection, T cell compositions, T cell product compositions, unstimulated T cell compositions, resting T cell compositions or any portion thereof may be resuspended in a nucleofection buffer. Nucleofection buffers of the disclosure include commercially-available nucleofection buffers. Nucleofection buffers of the disclosure may be optimized to yield cells that have greater viability, nucleofect with higher efficiency, exhibit greater viability post-nucleofection, display a more desirable cell phenotype, and/or greater/faster expansion upon addition of expansion technologies. Nucleofection buffers of the disclosure may include, but are not limited to, PBS, HBSS, OptiMEM, BTXpress, Amaxa Nucleofector, Human T cell nucleofection buffer and any combination thereof. Nucleofection buffers of the disclosure may comprise one or more supplemental factors to yield cells that have greater viability, nucleofect with higher efficiency, exhibit greater viability post-nucleofection, display a more desirable cell phenotype, and/or greater/faster expansion upon addition of expansion technologies. Exemplary supplemental factors include, but are not limited to, recombinant human cytokines, chemokines, interleukins and any combination thereof. Exemplary cytokines, chemokines, and interleukins include, but are not limited to, IL2, IL7, IL12, IL15, IL21, IL1 IL3, IL4, IL5, IL6, IL8, CXCL8, IL9, IL10, IL11, IL13, IL14, IL16, IL17, IL18, IL19, IL20, IL22, IL23, IL25, IL26, IL27, IL28, IL29, IL30, IL31, IL32, IL33, IL35, IL36, GM-CSF, IFN-gamma, IL-1 alpha/IL-1F1, IL-1 beta/IL-1F2, IL-12 p70, IL-12/IL-35 p35, IL-13, IL-17/IL-17A, IL-17A/F Heterodimer, IL-17F, IL-18/IL-1F4, IL-23, IL-24, IL-32, IL-32 beta, IL-32 gamma, IL-33, LAP (TGF-beta 1), Lymphotoxin-alpha/TNF-beta, TGF-beta, TNF-alpha, TRANCE/TNFSF11/RANK L and any combination thereof. Exemplary supplemental factors include, but are not limited to, salts, minerals, metabolites or any combination thereof. Exemplary salts, minerals, and metabolites include, but are not limited to, HEPES, Nicotinamide, Heparin, Sodium Pyruvate, L-Glutamine, MEM Non-Essential Amino Acid Solution, Ascorbic Acid, Nucleosides, FBS/FCS, Human serum, serum-substitute, anti-biotics, pH adjusters, Earle's Salts, 2-Mercaptoethanol, Human transferrin, Recombinant human insulin, Human serum albumin, Nucleofector PLUS Supplement, KCL, MgCl2, Na2HPO4, NAH2PO4, Sodium lactobionate, Manitol, Sodium succinate, Sodium Chloride, CINa, Glucose, Ca(NO3)2, Tris/HCl, K2HPO4, KH2PO4, Polyethylenimine, Poly-ethylene-glycol, Poloxamer 188, Poloxamer 181, Poloxamer 407, Poly-vinylpyrrolidone, Pop313, Crown-5, and any combination thereof. Exemplary supplemental factors include, but are not limited to, media such as PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium and any combination thereof. Exemplary supplemental factors include, but are not limited to, inhibitors of cellular DNA sensing, metabolism, differentiation, signal transduction, the apoptotic pathway and combinations thereof. Exemplary inhibitors include, but are not limited to, inhibitors of TLR9, MyD88, IRAK, TRAF6, TRAF3, IRF-7, NF-KB, Type 1 Interferons, pro-inflammatory cytokines, cGAS, STING, Sec5, TBK1, IRF-3, RNA pol III, RIG-1, IPS-1, FADD, RIP1, TRAF3, AIM2, ASC, Caspase1, Pro-IL1B, PI3K, Akt, Wnt3A, inhibitors of glycogen synthase kinase-3β (GSK-3 β) (e.g. TWS119), Bafilomycin, Chloroquine, Quinacrine, AC-YVAD-CMK, Z-VAD-FMK, Z-IETD-FMK and any combination thereof. Exemplary supplemental factors include, but are not limited to, reagents that modify or stabilize one or more nucleic acids in a way to enhance cellular delivery, enhance nuclear delivery or transport, enhance the facilitated transport of nucleic acid into the nucleus, enhance degradation of epi-chromosomal nucleic acid, and/or decrease DNA-mediated toxicity. Exemplary reagents that modify or stabilize one or more nucleic acids include, but are not limited to, pH modifiers, DNA-binding proteins, lipids, phospholipids, CaPO4, net neutral charge DNA binding peptides with or without NLS sequences, TREX1 enzyme, and any combination thereof.

Transposition reagents, including a transposon and a transposase, may be added to a nucleofection reaction of the disclosure prior to, simultaneously with, or after an addition of cells to a nucleofection buffer (optionally, contained within a nucleofection reaction vial or cuvette). Transposons of the disclosure may comprise plasmid DNA, linearized plasmid DNA, a PCR product, DOGGYBONE™ DNA, an mRNA template, a single or double-stranded DNA, a protein-nucleic acid combination or any combination thereof. Transposons of the disclosure may comprised one or more sequences that encode one or more TTAA site(s), one or more inverted terminal repeat(s) (ITRs), one or more long terminal repeat(s) (LTRs), one or more insulator(s), one or more promotor(s), one or more full-length or truncated gene(s), one or more polyA signal(s), one or more self-cleaving 2A peptide cleavage site(s), one or more internal ribosome entry site(s) (IRES), one or more enhancer(s), one or more regulator(s), one or more replication origin(s), and any combination thereof.

Transposons of the disclosure may comprise one or more sequences that encode one or more full-length or truncated gene(s). Full-length and/or truncated gene(s) introduced by transposons of the disclosure may encode one or more of a signal peptide, a CARTyrin, an anti-PSMA CARTyrin, a Centyrin, a PSMA-specific Centryin, a hinge, a transmembrane domain, a costimulatory domain, a chimeric antigen receptor (CAR), a chimeric T-cell receptor (CAR-T, a CARTyrin or an anti-PSMA CARTyrin), a receptor, a ligand, a cytokine, a drug resistance gene, a tumor antigen, an allo or auto antigen, an enzyme, a protein, a peptide, a poly-peptide, a fluorescent protein, a mutein or any combination thereof.

Transposons of the disclosure may be prepared in water, TAE, TBE, PBS, HBSS, media, a supplemental factor of the disclosure or any combination thereof.

Transposons of the disclosure may be designed to optimize clinical safety and/or improve manufacturability. As a non-limiting example, transposons of the disclosure may be designed to optimize clinical safety and/or improve manufacturability by eliminating unnecessary sequences or regions and/or including a non-antibiotic selection marker. Transposons of the disclosure may or may not be GMP grade.

Transposase enzymes of the disclosure may be encoded by one or more sequences of plasmid DNA, mRNA, protein, protein-nucleic acid combination or any combination thereof.

Transposase enzymes of the disclosure may be prepared in water, TAE, TBE, PBS, HBSS, media, a supplemental factor of the disclosure or any combination thereof. Transposase enzymes of the disclosure or the sequences/constructs encoding or delivering them may or may not be GMP grade.

Transposons and transposase enzymes of the disclosure may be delivered to a cell by any means.

Although compositions and methods of the disclosure include delivery of a transposon and/or transposase of the disclosure to a cell by plasmid DNA (pDNA), the use of a plasmid for delivery may allow the transposon and/or transposase to be integrated into the chromosomal DNA of the cell, which may lead to continued transposase expression. Accordingly, transposon and/or transposase enzymes of the disclosure may be delivered to a cell as either mRNA or protein to remove any possibility for chromosomal integration.

Transposons and transposases of the disclosure may be pre-incubated alone or in combination with one another prior to the introduction of the transposon and/or transposase into a nucleofection reaction. The absolute amounts of each of the transposon and the transposase, as well as the relative amounts, e.g., a ratio of transposon to transposase may be optimized.

Following preparation of nucleofection reaction, optionally, in a vial or cuvette, the reaction may be loaded into a nucleofector apparatus and activated for delivery of an electric pulse according to the manufacturer's protocol. Electric pulse conditions used for delivery of a transposon and/or a transposase of the disclosure (or a sequence encoding a transposon and/or a transposase of the disclosure) to a cell may be optimized for yielding cells with enhanced viability, higher nucleofection efficiency, greater viability post-nucleofection, desirable cell phenotype, and/or greater/faster expansion upon addition of expansion technologies. When using Amaxa nucleofector technology, each of the various nucleofection programs for the Amaxa 2B or 4D nucleofector are contemplated.

Following a nucleofection reaction of the disclosure, cells may be gently added to a cell medium. For example, when T cells undergo the nucleofection reaction, the T cells may be added to a T cell medium. Post-nucleofection cell media of the disclosure may comprise any one or more commercially-available media. Post-nucleofection cell media of the disclosure (including post-nucleofection T cell media of the disclosure) may be optimized to yield cells with greater viability, higher nucleofection efficiency, exhibit greater viability post-nucleofection, display a more desirable cell phenotype, and/or greater/faster expansion upon addition of expansion technologies. Post-nucleofection cell media of the disclosure (including post-nucleofection T cell media of the disclosure) may comprise PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium and any combination thereof. Post-nucleofection cell media of the disclosure (including post-nucleofection T cell media of the disclosure) may comprise one or more supplemental factors of the disclosure to enhance viability, nucleofection efficiency, viability post-nucleofection, cell phenotype, and/or greater/faster expansion upon addition of expansion technologies. Exemplary supplemental factors include, but are not limited to, recombinant human cytokines, chemokines, interleukins and any combination thereof. Exemplary cytokines, chemokines, and interleukins include, but are not limited to, IL2, IL7, IL12, IL15, IL21, IL1, IL3, IL4, IL5, IL6, IL8, CXCL8, IL9, IL10, IL11, IL13, IL14, IL16, IL17, IL18, IL19, IL20, IL22, IL23, IL25, IL26, IL27, IL28, IL29, IL30, IL31, IL32, IL33, IL35, IL36, GM-CSF, IFN-gamma, IL-1 alpha/IL-1F1, IL-1 beta/IL-1F2, IL-12 p70, IL-12/IL-35 p35, IL-13, IL-17/IL-17A, IL-17A/F Heterodimer, IL-17F, IL-18/IL-1F4, IL-23, IL-24, IL-32, IL-32 beta, IL-32 gamma, IL-33, LAP (TGF-beta 1), Lymphotoxin-alpha/TNF-beta, TGF-beta, TNF-alpha, TRANCE/TNFSF11/RANK L and any combination thereof. Exemplary supplemental factors include, but are not limited to, salts, minerals, metabolites or any combination thereof. Exemplary salts, minerals, and metabolites include, but are not limited to, HEPES, Nicotinamide, Heparin, Sodium Pyruvate, L-Glutamine, MEM Non-Essential Amino Acid Solution, Ascorbic Acid, Nucleosides, FBS/FCS, Human serum, serum-substitute, anti-biotics, pH adjusters, Earle's Salts, 2-Mercaptoethanol, Human transferrin, Recombinant human insulin, Human serum albumin, Nucleofector PLUS Supplement, KCL, MgCl2, Na2HPO4, NAH2PO4, Sodium lactobionate, Manitol, Sodium succinate, Sodium Chloride, CINa, Glucose, Ca(NO3)2, Tris/HCl, K2HPO4, KH2PO4, Polyethylenimine, Poly-ethylene-glycol, Poloxamer 188, Poloxamer 181, Poloxamer 407, Poly-vinylpyrrolidone, Pop313, Crown-5, and any combination thereof. Exemplary supplemental factors include, but are not limited to, media such as PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium and any combination thereof. Exemplary supplemental factors include, but are not limited to, inhibitors of cellular DNA sensing, metabolism, differentiation, signal transduction, the apoptotic pathway and combinations thereof. Exemplary inhibitors include, but are not limited to, inhibitors of TLR9, MyD88, IRAK, TRAF6, TRAF3, IRF-7, NF-KB, Type 1 Interferons, pro-inflammatory cytokines, cGAS, STING, Sec5, TBK1, IRF-3, RNA pol III, RIG-1, IPS-1, FADD, RIP1, TRAF3, AIM2, ASC, Caspase1, Pro-IL1B, PI3K, Akt, Wnt3A, inhibitors of glycogen synthase kinase-3β (GSK-3β) (e.g. TWS119), Bafilomycin, Chloroquine, Quinacrine, AC-YVAD-CMK, Z-VAD-FMK, Z-IETD-FMK and any combination thereof. Exemplary supplemental factors include, but are not limited to, reagents that modify or stabilize one or more nucleic acids in a way to enhance cellular delivery, enhance nuclear delivery or transport, enhance the facilitated transport of nucleic acid into the nucleus, enhance degradation of epi-chromosomal nucleic acid, and/or decrease DNA-mediated toxicity. Exemplary reagents that modify or stabilize one or more nucleic acids include, but are not limited to, pH modifiers, DNA-binding proteins, lipids, phospholipids, CaPO4, net neutral charge DNA binding peptides with or without NLS sequences, TREX1 enzyme, and any combination thereof.

Post-nucleofection cell media of the disclosure (including post-nucleofection T cell media of the disclosure) may be used at room temperature or pre-warmed to, for example to between 32° C. to 37° C., inclusive of the endpoints. Post-nucleofection cell media of the disclosure (including post-nucleofection T cell media of the disclosure) may be pre-warmed to any temperature that maintains or enhances cell viability and/or expression of a transposon or portion thereof of the disclosure.

Post-nucleofection cell media of the disclosure (including post-nucleofection T cell media of the disclosure) may be contained in tissue culture flasks or dishes, G-Rex flasks, Bioreactor or cell culture bags, or any other standard receptacle. Post-nucleofection cell cultures of the disclosure (including post-nucleofection T cell cultures of the disclosure) may be may be kept still, or, alternatively, they may be perturbed (e.g. rocked, swirled, or shaken).

Post-nucleofection cell cultures may comprise genetically-modified cells. Post-nucleofection T cell cultures may comprise genetically-modified T cells. Genetically modified cells of the disclosure may be either rested for a defined period of time or stimulated for expansion by, for example, the addition of a T Cell Expander technology. In certain embodiments, genetically modified cells of the disclosure may be either rested for a defined period of time or immediately stimulated for expansion by, for example, the addition of a T Cell Expander technology. Genetically modified cells of the disclosure may be rested to allow them sufficient time to acclimate, time for transposition to occur, and/or time for positive or negative selection, resulting in cells with enhanced viability, higher nucleofection efficiency, greater viability post-nucleofection, desirable cell phenotype, and/or greater/faster expansion upon addition of expansion technologies. Genetically modified cells of the disclosure may be rested, for example, for 1, 2, 3, 4, 5, 6, 7, 8, 9, 10, 11, 12, 13, 14, 15, 16, 17, 18, 19, 20, 21, 22, 23, 24, or more hours. In certain embodiments, genetically modified cells of the disclosure may be rested, for example, for an overnight. In certain aspects, an overnight is about 12 hours. Genetically modified cells of the disclosure may be rested, for example, for 1, 2, 3, 4, 5, 6, 7, 8, 9, 10, 11, 12, 13, 14 or more days.

Genetically modified cells of the disclosure may be selected following a nucleofection reaction and prior to addition of an expander technology. For optimal selection of genetically-modified cells, the cells may be allowed to rest in a post-nucleofection cell medium for at least 2-14 days to facilitate identification of modified cells (e.g., differentiation of modified from non-modified cells).

As early as 24-hours post-nucleofection, expression of a Centyrin or CARTyrin and selection marker of the disclosure may be detectable in modified T cells upon successful nucleofection of a transposon of the disclosure. Due to epi-chromosomal expression of the transposon, expression of a selection marker alone may not differentiate modified T cells (those cells in which the transposon has been successfully integrated) from unmodified T cells (those cells in which the transposon was not successfully integrated). When epi-chromosomal expression of the transposon obscures the detection of modified cells by the selection marker, the nucleofected cells (both modified and unmodified cells) may be rested for a period of time (e.g. 2-14 days) to allow the cells to cease expression or lose all epi-chromosomal transposon expression. Following this extended resting period, only modified T cells should remain positive for expression of selection marker. The length of this extended resting period may be optimized for each nucleofection reaction and selection process. When epi-chromosomal expression of the transposon obscures the detection of modified cells by the selection marker, selection may be performed without this extended resting period, however, an additional selection step may be included at a later time point (e.g. either during or after the expansion stage).

Selection of genetically modified cells of the disclosure may be performed by any means. In certain embodiments of the methods of the disclosure, selection of genetically modified cells of the disclosure may be performed by isolating cells expressing a specific selection marker. Selection markers of the disclosure may be encoded by one or more sequences in the transposon. Selection markers of the disclosure may be expressed by the modified cell as a result of successful transposition (i.e., not encoded by one or more sequences in the transposon). In certain embodiments, genetically modified cells of the disclosure contain a selection marker that confers resistance to a deleterious compound of the post-nucleofection cell medium. The deleterious compound may comprise, for example, an antibiotic or a drug that, absent the resistance conferred by the selection marker to the modified cells, would result in cell death. Exemplary selection markers include, but are not limited to, wild type (WT) or mutant forms of one or more of the following genes: neo, DHFR, TYMS, ALDH, MDR1, MGMT, FANCF, RAD51C, GCS, and NKX2.2. Exemplary selection markers include, but are not limited to, a surface-expressed selection marker or surface-expressed tag may be targeted by Ab-coated magnetic bead technology or column selection, respectively. A cleavable tag such as those used in protein purification may be added to a selection marker of the disclosure for efficient column selection, washing, and elution. In certain embodiments, selection markers of the disclosure are not expressed by the modified cells (including modified T cells) endogenously and, therefore, may be useful in the physical isolation of modified cells (by, for example, cell sorting techniques). Exemplary selection markers of the disclosure are not expressed by the modified cells (including modified T cells) endogenously include, but are not limited to, full-length, mutated, or truncated forms of CD271, CD19 CD52, CD34, RQR8, CD22, CD20, CD33 and any combination thereof.

Genetically modified cells of the disclosure may be selective expanded following a nucleofection reaction. In certain embodiments, modified T cells comprising a CARTyrin may be selectively expanded by CARTyrin stimulation. Modified T cells comprising a CARTyrin may be stimulated by contact with a target-covered reagent (e.g. a tumor line or a normal cell line expressing a target or expander beads covered in a target). Alternatively, modified T cells comprising a CARTyrin may be stimulated by contact with an irradiated tumor cell, an irradiated allogeneic normal cell, an irradiated autologous PBMC. To minimize contamination of cell product compositions of the disclosure with a target-expressing cell used for stimulation, for example, when the cell product composition may be administered directly to a subject, the stimulation may be performed using expander beads coated with CARTyrin target protein. Selective expansion of modified T cells comprising a CARTyrin by CARTyrin stimulation may be optimized to avoid functionally-exhausting the modified T-cells.

Selected genetically-modified cells of the disclosure may be cryopreserved, rested for a defined period of time, or stimulated for expansion by the addition of a Cell Expander technology. Selected genetically-modified cells of the disclosure may be cryopreserved, rested for a defined period of time, or immediately stimulated for expansion by the addition of a Cell Expander technology. When the selected genetically-modified cells are T cells, the T cells may be stimulated for expansion by the addition of a T-Cell Expander technology. Selected genetically modified cells of the disclosure may be rested, for example, for 1, 2, 3, 4, 5, 6, 7, 8, 9, 10, 11, 12, 13, 14, 15, 16, 17, 18, 19, 20, 21, 22, 23, 24, or more hours. In certain embodiments, selected genetically modified cells of the disclosure may be rested, for example, for an overnight. In certain aspects, an overnight is about 12 hours. Selected genetically modified cells of the disclosure may be rested, for example, for 1, 2, 3, 4, 5, 6, 7, 8, 9, 10, 11, 12, 13, 14 or more days. Selected genetically modified cells of the disclosure may be rested for any period of time resulting in cells with enhanced viability, higher nucleofection efficiency, greater viability post-nucleofection, desirable cell phenotype, and/or greater/faster expansion upon addition of expansion technologies.

Selected genetically-modified cells (including selected genetically-modified T cells of the disclosure) may be cryopreserved using any standard cryopreservation method, which may be optimized for storing and/or recovering human cells with high recovery, viability, phenotype, and/or functional capacity. Cryopreservation methods of the disclosure may include commercially-available cryopreservation media and/or protocols.

A transposition efficiency of selected genetically-modified cells (including selected genetically-modified T cells of the disclosure) may be assessed by any means. For example, prior to the application of an expander technology, expression of the transposon by selected genetically-modified cells (including selected genetically-modified T cells of the disclosure) may be measured by fluorescence-activated cell sorting (FACS). Determination of a transposition efficiency of selected genetically-modified cells (including selected genetically-modified T cells of the disclosure) may include determining a percentage of selected cells expressing the transposon (e.g. a CARTyrin). Alternatively, or in addition, a purity of T cells, a Mean Fluorescence Intensity (MFI) of the transposon expression (e.g. CARTyrin expression), an ability of a CARTyrin (delivered in the transposon) to mediate degranulation and/or killing of a target cell expressing the CARTyrin ligand, and/or a phenotype of selected genetically-modified cells (including selected genetically-modified T cells of the disclosure) may be assessed by any means.

Cell product compositions of the disclosure may be released for administration to a subject upon meeting certain release criteria. Exemplary release criteria may include, but are not limited to, a particular percentage of modified, selected and/or expanded T cells expressing detectable levels of a CARTyrin on the cell surface.

### Genetic Modification of an Autologous T Cell Product Composition

Genetically-modified cells (including genetically-modified T cells) of the disclosure may be expanded using an expander technology. Expander technologies of the disclosure may comprise a commercially-available expander technology. Exemplary expander technologies of the disclosure include stimulation a genetically-modified T cell of the disclosure via the TCR. While all means for stimulation of a genetically-modified T cell of the disclosure are contemplated, stimulation a genetically-modified T cell of the disclosure via the TCR is a preferred method, yielding a product with a superior level of killing capacity.

To stimulate a genetically-modified T cell of the disclosure via the TCR, Thermo Expander DynaBeads may be used at a 3:1 bead to T cell ratio. If the expander beads are not biodegradable, the beads may be removed from the expander composition. For example, the beads may be removed from the expander composition after about 5 days. To stimulate a genetically-modified T cell of the disclosure via the TCR, a Miltenyi T Cell Activation/Expansion Reagent may be used. To stimulate a genetically-modified T cell of the disclosure via the TCR, StemCell Technologies' ImmunoCult Human CD3/CD28 or CD3/CD28/CD2 T Cell Activator Reagent may be used. This technology may be preferred since the soluble tetrameric antibody complexes would degrade after a period and would not require removal from the process.

Artificial antigen presenting cells (APCs) may be engineered to co-express the target antigen and may be used to stimulate a cell or T-cell of the disclosure through a TCR and/or CARTyrin of the disclosure. Artificial APCs may comprise or may be derived from a tumor cell line (including, for example, the immortalized myelogenous leukemia line K562) and may be engineered to co-express multiple costimulatory molecules or technologies (such as CD28, 4-1BBL, CD64, mbIL-21, mbIL-15, CAR target molecule, etc.). When artificial APCs of the disclosure are combined with costimulatory molecules, conditions may be optimized to prevent the development or emergence of an undesirable phenotype and functional capacity, namely terminally-differentiated effector T cells.

Irradiated PBMCs (auto or allo) may express some target antigens, such as CD19, and may be used to stimulate a cell or T-cell of the disclosure through a TCR and/or CARTyrin of the disclosure. Alternatively, or in addition, irradiated tumor cells may express some target antigens and may be used to stimulate a cell or T-cell of the disclosure through a TCR and/or CARTyrin of the disclosure.

Plate-bound and/or soluble anti-CD3, anti-CD2 and/or anti-CD28 stimulate may be used to stimulate a cell or T-cell of the disclosure through a TCR and/or CARTyrin of the disclosure.

Antigen-coated beads may display target protein and may be used to stimulate a cell or T-cell of the disclosure through a TCR and/or CARTyrin of the disclosure. Alternatively, or in addition, expander beads coated with a CARTyrin target protein may be used to stimulate a cell or T-cell of the disclosure through a TCR and/or CARTyrin of the disclosure.

Expansion methods drawn to stimulation of a cell or T-cell of the disclosure through the TCR or CARTyrin and via surface-expressed CD2, CD3, CD28, 4-1BB, and/or other markers on genetically-modified T cells.

An expansion technology may be applied to a cell of the disclosure immediately post-nucleofection until approximately 24 hours post-nucleofection. While various cell media may be used during an expansion procedure, a desirable T Cell Expansion Media of the disclosure may yield cells with, for example, greater viability, cell phenotype, total expansion, or greater capacity for in vivo persistence, engraftment, and/or CAR-mediated killing. Cell media of the disclosure may be optimized to improve/enhance expansion, phenotype, and function of genetically-modified cells of the disclosure. A preferred phenotype of expanded T cells may include a mixture of T stem cell memory, T central, and T effector memory cells. Expander Dynabeads may yield mainly central memory T cells which may lead to superior performance in the clinic.

Exemplary T cell expansion media of the disclosure may include, in part or in total, PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium, or any combination thereof. T cell expansion media of the disclosure may further include one or more supplemental factors. Supplemental factors that may be included in a T cell expansion media of the disclosure enhance viability, cell phenotype, total expansion, or increase capacity for in vivo persistence, engraftment, and/or CARTyrin-mediated killing. Supplemental factors that may be included in a T cell expansion media of the disclosure include, but are not limited to, recombinant human cytokines, chemokines, and/or interleukins such as IL2, IL7, IL12, IL15, IL21, IL1, IL3, IL4, IL5, IL6, IL8, CXCL8, IL9, IL10, IL11, IL13, IL14, IL16, IL17, IL18, IL19, IL20, IL22, IL23, IL25, IL26, IL27, IL28, IL29, IL30, IL31, IL32, IL33, IL35, IL36, GM-CSF, IFN-gamma, IL-1 alpha/IL-1F1, IL-1 beta/IL-1F2, IL-12 p70, IL-12/IL-35 p35, IL-13, IL-17/IL-17A, IL-17A/F Heterodimer, IL-17F, IL-18/IL-1F4, IL-23, IL-24, IL-32, IL-32 beta, IL-32 gamma, IL-33, LAP (TGF-beta 1), Lymphotoxin-alpha/TNF-beta, TGF-beta, TNF-alpha, TRANCE/TNFSF11/RANK L, or any combination thereof. Supplemental factors that may be included in a T cell expansion media of the disclosure include, but are not limited to, salts, minerals, and/or metabolites such as HEPES, Nicotinamide, Heparin, Sodium Pyruvate, L-Glutamine, MEM Non-Essential Amino Acid Solution, Ascorbic Acid, Nucleosides, FBS/FCS, Human serum, serum-substitute, anti-biotics, pH adjusters, Earle's Salts, 2-Mercaptoethanol, Human transferrin, Recombinant human insulin, Human serum albumin, Nucleofector PLUS Supplement, KCL, MgCl2, Na2HPO4, NAH2PO4, Sodium lactobionate, Manitol, Sodium succinate, Sodium Chloride, CINa, Glucose, Ca(NO3)2, Tris/HCl, K2HPO4, KH2PO4, Polyethylenimine, Poly-ethylene-glycol, Poloxamer 188, Poloxamer 181, Poloxamer 407, Poly-vinylpyrrolidone, Pop313, Crown-5 or any combination thereof. Supplemental factors that may be included in a T cell expansion media of the disclosure include, but are not limited to, inhibitors of cellular DNA sensing, metabolism, differentiation, signal transduction, and/or the apoptotic pathway such as inhibitors of TLR9, MyD88, IRAK, TRAF6, TRAF3, IRF-7, NF-KB, Type 1 Interferons, pro-inflammatory cytokines, cGAS, STING, Sec5, TBK1, IRF-3, RNA pol III, RIG-1, IPS-1, FADD, RIP1, TRAF3, AIM2, ASC, Caspase1, Pro-IL1B, PI3K, Akt, Wnt3A, inhibitors of glycogen synthase kinase-3β (GSK-3 β) (e.g. TWS119), Bafilomycin, Chloroquine, Quinacrine, AC-YVAD-CMK, Z-VAD-FMK, Z-IETD-FMK, or any combination thereof.

Supplemental factors that may be included in a T cell expansion media of the disclosure include, but are not limited to, reagents that modify or stabilize nucleic acids in a way to enhance cellular delivery, enhance nuclear delivery or transport, enhance the facilitated transport of nucleic acid into the nucleus, enhance degradation of epi-chromosomal nucleic acid, and/or decrease DNA-mediated toxicity, such as pH modifiers, DNA-binding proteins, lipids, phospholipids, CaPO4, net neutral charge DNA binding peptides with or without NLS sequences, TREX1 enzyme, or any combination thereof.

Genetically-modified cells of the disclosure may be selected during the expansion process by the use of selectable drugs or compounds. For example, in certain embodiments, when a transposon of the disclosure may encode a selection marker that confers to genetically-modified cells resistance to a drug added to the culture medium, selection may occur during the expansion process and may require approximately 1-14 days of culture for selection to occur. Examples of drug resistance genes that may be used as selection markers encoded by a transposon of the disclosure, include, but are not limited to, wild type (WT) or mutant forms of the genes neo, DHFR, TYMS, ALDH, MDR1, MGMT, FANCF, RAD51C, GCS, NKX2.2, or any combination thereof. Examples of corresponding drugs or compounds that may be added to the culture medium to which a selection marker may confer resistance include, but are not limited to, G418, Puromycin, Ampicillin, Kanamycin, Methotrexate, Mephalan, Temozolomide, Vincristine, Etoposide, Doxorubicin, Bendamustine, Fludarabine, Aredia (Pamidronate Disodium), Becenum (Carmustine), BiCNU (Carmustine), Bortezomib, Carfilzomib, Carmubris (Carmustine), Carmustine, Clafen (Cyclophosphamide), Cyclophosphamide, Cytoxan (Cyclophosphamide), Daratumumab, Darzalex (Daratumumab), Doxil (Doxorubicin Hydrochloride Liposome), Doxorubicin Hydrochloride Liposome, Dox-SL (Doxorubicin Hydrochloride Liposome), Elotuzumab, Empliciti (Elotuzumab), Evacet (Doxorubicin Hydrochloride Liposome), Farydak (Panobinostat), Ixazomib Citrate, Kyprolis (Carfilzomib), Lenalidomide, LipoDox (Doxorubicin Hydrochloride Liposome), Mozobil (Plerixafor), Neosar (Cyclophosphamide), Ninlaro (Ixazomib Citrate), Pamidronate Disodium, Panobinostat, Plerixafor, Pomalidomide, Pomalyst (Pomalidomide), Revlimid (Lenalidomide), Synovir (Thalidomide), Thalidomide, Thalomid (Thalidomide), Velcade (Bortezomib), Zoledronic Acid, Zometa (Zoledronic Acid), or any combination thereof.

A T-Cell Expansion process of the disclosure may occur in a cell culture bag in a WAVE Bioreactor, a G-Rex flask, or in any other suitable container and/or reactor.

A cell or T-cell culture of the disclosure may be kept steady, rocked, swirled, or shaken.

A cell or T-cell expansion process of the disclosure may optimize certain conditions, including, but not limited to culture duration, cell concentration, schedule for T cell medium addition/removal, cell size, total cell number, cell phenotype, purity of cell population, percentage of genetically-modified cells in growing cell population, use and composition of supplements, the addition/removal of expander technologies, or any combination thereof.

A cell or T-cell expansion process of the disclosure may continue until a predefined endpoint prior to formulation of the resultant expanded cell population. For example, a cell or T-cell expansion process of the disclosure may continue for a predetermined amount of time: at least, 2, 4, 6, 8, 10, 12, 14, 16, 18, 20, 22, 24 hours; at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days; at least 1, 2, 3, 4, 5, 6, 7, 8, 9, 10, 11, 12 weeks; at least 1, 2, 3, 4, 5, 6, months, or at least 1 year. A cell or T-cell expansion process of the disclosure may continue until the resultant culture reaches a predetermined overall cell density: 1, 10, 100, 1000, 104, 105, 106, 107, 108, 109, 1010 cells per volume (μl, ml, L) or any density in between. A cell or T-cell expansion process of the disclosure may continue until the genetically-modified cells of a resultant culture demonstrate a predetermined level of expression of a transposon of the disclosure: 1%, 10%, 20%, 30%, 40%, 50%, 60%, 70%, 80%, 90%, or 100% or any percentage in between of a threshold level of expression (a minimum, maximum or mean level of expression indicating the resultant genetically-modified cells are clinically-efficacious). A cell or T-cell expansion process of the disclosure may continue until the proportion of genetically-modified cells of a resultant culture to the proportion of unmodified cells reaches a predetermined threshold: at least 1:10, 1:9, 1:8, 1:7, 1:6, 1:5, 1:4, 1:3, 1:2, 1:1, 2:1, 2:1, 4:1, 5:1, 6:1, 7:1, 8:1, 9:1 10:1 or any ratio in between.

### Analysis of Genetically-Modified Autologous T Cells for Release

A percentage of genetically-modified cells may be assessed during or after an expansion process of the disclosure. Cellular expression of a transposon by a genetically-modified cell of the disclosure may be measured by fluorescence-activated cell sorting (FACS). For example, FACS may be used to determine a percentage of cells or T cells expressing a CARTyrin of the disclosure. Alternatively, or in addition, a purity of genetically-modified cells or T cells, the Mean Fluorescence Intensity (MFI) of a CARTyrin expressed by a genetically-modified cell or T cell of the disclosure, an ability of the CARTyrin to mediate degranulation and/or killing of a target cell expressing the CARTyrin ligand, and/or a phenotype of CARTyrin+ T cells may be assessed.

Compositions of the disclosure intended for administration to a subject may be required to meet one or more “release criteria” that indicate that the composition is safe and efficacious for formulation as a pharmaceutical product and/or administration to a subject. Release criteria may include a requirement that a composition of the disclosure (e.g. a T-cell product of the disclosure) comprises a particular percentage of T cells expressing detectable levels of a CARTyrin of the disclosure on their cell surface.

The expansion process should be continued until a specific criterion has been met (e.g. achieving a certain total number of cells, achieving a particular population of memory cells, achieving a population of a specific size).

Certain criterion signal a point at which the expansion process should end. For example, cells should be formulated, reactivated, or cryopreserved once they reach a cell size of 300 fL (otherwise, cells reaching a size above this threshold may start to die). Cryopreservation immediately once a population of cells reaches an average cell size of less than 300 fL may yield better cell recovery upon thawing and culture because the cells haven't yet reached a fully quiescent state prior to cryopreservation (a fully quiescent size is approximately 180 fL). Prior to expansion, T cells of the disclosure may have a cell size of about 180 fL, but may more than quadruple their cell size to approximately 900 fL at 3 days post-expansion. Over the next 6-12 days, the population of T-cells will slowly decrease cell size to full quiescence at 180 fL.

A process for preparing a cell population for formulation may include, but is not limited to the steps of, concentrating the cells of the cell population, washing the cells, and/or further selection of the cells via drug resistance or magnetic bead sorting against a particular surface-expressed marker. A process for preparing a cell population for formulation may further include a sorting step to ensure the safety and purity of the final product. For example, if a tumor cell from a patient has been used to stimulate a genetically-modified T-cell of the disclosure or that have been genetically-modified in order to stimulate a genetically-modified T-cell of the disclosure that is being prepared for formulation, it is critical that no tumor cells from the patient are included in the final product.

Cell Product Infusion and/or Cryopreservation for Infusion

A pharmaceutical formulation of the disclosure may be distributed into bags for infusion, cryopreservation, and/or storage.

A pharmaceutical formulation of the disclosure may be cryopreserved using a standard protocol and, optionally, an infusible cryopreservation medium. For example, a DMSO free cryopreservant (e.g. CryoSOfree™ DMSO-free Cryopreservation Medium) may be used to reduce freezing-related toxicity. A cryopreserved pharmaceutical formulation of the disclosure may be stored for infusion to a patient at a later date. An effective treatment may require multiple administrations of a pharmaceutical formulation of the disclosure and, therefore, pharmaceutical formulations may be packaged in pre-aliquoted “doses” that may be stored frozen but separated for thawing of individual doses.

A pharmaceutical formulation of the disclosure may be stored at room temperature. An effective treatment may require multiple administrations of a pharmaceutical formulation of the disclosure and, therefore, pharmaceutical formulations may be packaged in pre-aliquoted “doses” that may be stored together but separated for administration of individual doses.

A pharmaceutical formulation of the disclosure may be archived for subsequent re-expansion and/or selection for generation of additional doses to the same patient in the case of an allogenic therapy who may need an administration at a future date following, for example, a remission and relapse of a condition.

### Formulations

As noted above, the disclosure provides for stable formulations, which preferably comprise a phosphate buffer with saline or a chosen salt, as well as preserved solutions and formulations containing a preservative as well as multi-use preserved formulations suitable for pharmaceutical or veterinary use, comprising at least one CARTyrin in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, polymers, or mixtures thereof in an aqueous diluent. Any suitable concentration or mixture can be used as known in the art, such as about 0.0015%, or any range, value, or fraction therein. Non-limiting examples include, no preservative, about 0.1-2% m-cresol (e.g., 0.2, 0.3. 0.4, 0.5, 0.9, 1.0%), about 0.1-3% benzyl alcohol (e.g., 0.5, 0.9, 1.1, 1.5, 1.9, 2.0, 2.5%), about 0.001-0.5% thimerosal (e.g., 0.005, 0.01), about 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the invention provides an article of manufacture, comprising packaging material and at least one vial comprising a solution of at least one CARTyrin with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising lyophilized at least one CARTyrin, and a second vial comprising an aqueous diluent of prescribed buffer or preservative, wherein said packaging material comprises a label that instructs a patient to reconstitute the at least one CARTyrin in the aqueous diluent to form a solution that can be held over a period of twenty-four hours or greater.

The at least one CARTyrin used in accordance with the present invention can be produced by recombinant means, including from mammalian cell or transgenic preparations, or can be purified from other biological sources, as described herein or as known in the art.

The range of at least one CARTyrin in the product of the present invention includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g., isotonicity agents, buffers, antioxidants, and preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably, the formulations of the present invention have a pH between about 6.8 and about 7.8. Preferred buffers include phosphate buffers, most preferably, sodium phosphate, particularly, phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants, such as polysorbate 20 or 80 or poloxamer 184 or 188, Pluronic® polyls, other block co-polymers, and chelators, such as EDTA and EGTA, can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations of the present invention can be prepared by a process which comprises mixing at least one CARTyrin and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one CARTyrin and preservative in an aqueous diluent is carried out using conventional dissolution and mixing procedures. To prepare a suitable formulation, for example, a measured amount of at least one CARTyrin in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed formulations can be provided to patients as clear solutions or as dual vials comprising a vial of lyophilized at least one CARTyrin that is reconstituted with a second vial containing water, a preservative and/or excipients, preferably, a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present claimed articles of manufacture are useful for administration over a period ranging from immediate to twenty-four hours or greater. Accordingly, the presently claimed articles of manufacture offer significant advantages to the patient. Formulations of the invention can optionally be safely stored at temperatures of from about 2° C. to about 40° C. and retain the biological activity of the protein for extended periods of time, thus allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of at least one CARTyrin of the invention can be prepared by a process that comprises mixing at least one CARTyrin in an aqueous diluent. Mixing is carried out using conventional dissolution and mixing procedures. To prepare a suitable diluent, for example, a measured amount of at least one CARTyrin in water or buffer is combined in quantities sufficient to provide the protein and, optionally, a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to patients as clear solutions or as dual vials comprising a vial of lyophilized at least one CARTyrin that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one CARTyrin that is reconstituted with a second vial containing the aqueous diluent. The clear solution in this case can be up to one liter or even larger in size, providing a large reservoir from which smaller portions of the at least one CARTyrin solution can be retrieved one or multiple times for transfer into smaller vials and provided by the pharmacy or clinic to their customers and/or patients.

Recognized devices comprising single vial systems include pen-injector devices for delivery of a solution, such as BD Pens, BD Autojector®, Humaject®, NovoPen®, BD®Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, e.g., as made or developed by Becton Dickinson (Franklin Lakes, N.J., www.bectondickenson.com), Disetronic (Burgdorf, Switzerland, www.disetronic.com; Bioject, Portland, Oreg. (www.bioject.com); National Medical Products, Weston Medical (Peterborough, UK, www.weston-medical.com), Medi-Ject Corp (Minneapolis, Minn., www.mediject.com), and similarly suitable devices. Recognized devices comprising a dual vial system include those pen-injector systems for reconstituting a lyophilized drug in a cartridge for delivery of the reconstituted solution, such as the HumatroPen®. Examples of other devices suitable include pre-filled syringes, auto-injectors, needle free injectors and needle free IV infusion sets.

The products presently claimed include packaging material. The packaging material provides, in addition to the information required by the regulatory agencies, the conditions under which the product can be used. The packaging material of the present invention provides instructions to the patient to reconstitute at least one CARTyrin in the aqueous diluent to form a solution and to use the solution over a period of 2-24 hours or greater for the two vial, wet/dry, product. For the single vial, solution product, the label indicates that such solution can be used over a period of 2-24 hours or greater. The presently claimed products are useful for human pharmaceutical product use.

The formulations of the present invention can be prepared by a process that comprises mixing at least one CARTyrin and a selected buffer, preferably, a phosphate buffer containing saline or a chosen salt. Mixing at least one CARTyrin and buffer in an aqueous diluent is carried out using conventional dissolution and mixing procedures. To prepare a suitable formulation, for example, a measured amount of at least one CARTyrin in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed stable or preserved formulations can be provided to patients as clear solutions or as dual vials comprising a vial of lyophilized CARTyrin that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

Other formulations or methods of stabilizing the CARTyrin may result in other than a clear solution of lyophilized powder comprising the CARTyrin. Among non-clear solutions are formulations comprising particulate suspensions, said particulates being a composition containing the CARTyrin in a structure of variable dimension and known variously as a microsphere, microparticle, nanoparticle, nanosphere, or liposome. Such relatively homogenous, essentially spherical, particulate formulations containing an active agent can be formed by contacting an aqueous phase containing the active agent and a polymer and a nonaqueous phase followed by evaporation of the nonaqueous phase to cause the coalescence of particles from the aqueous phase as taught in U.S. Pat. No. 4,589,330. Porous microparticles can be prepared using a first phase containing active agent and a polymer dispersed in a continuous solvent and removing said solvent from the suspension by freeze-drying or dilution-extraction-precipitation as taught in U.S. Pat. No. 4,818,542. Preferred polymers for such preparations are natural or synthetic copolymers or polymers selected from the group consisting of gelatin agar, starch, arabinogalactan, albumin, collagen, polyglycolic acid, polylactic aced, glycolide-L(−) lactide poly(episilon-caprolactone, poly(epsilon-caprolactone-CO-lactic acid), poly(epsilon-caprolactone-CO-glycolic acid), poly(β-hydroxy butyric acid), polyethylene oxide, polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly preferred polymers are polyesters, such as polyglycolic acid, polylactic aced, glycolide-L(−) lactide poly(episilon-caprolactone, poly(epsilon-caprolactone-CO-lactic acid), and poly(epsilon-caprolactone-CO-glycolic acid. Solvents useful for dissolving the polymer and/or the active include: water, hexafluoroisopropanol, methylenechloride, tetrahydrofuran, hexane, benzene, or hexafluoroacetone sesquihydrate. The process of dispersing the active containing phase with a second phase may include pressure forcing said first phase through an orifice in a nozzle to affect droplet formation.

Dry powder formulations may result from processes other than lyophilization, such as by spray drying or solvent extraction by evaporation or by precipitation of a crystalline composition followed by one or more steps to remove aqueous or nonaqueous solvent. Preparation of a spray-dried CARTyrin preparation is taught in U.S. Pat. No. 6,019,968. The CARTyrin-based dry powder compositions may be produced by spray drying solutions or slurries of the CARTyrin and, optionally, excipients, in a solvent under conditions to provide a respirable dry powder. Solvents may include polar compounds, such as water and ethanol, which may be readily dried. CARTyrin stability may be enhanced by performing the spray drying procedures in the absence of oxygen, such as under a nitrogen blanket or by using nitrogen as the drying gas. Another relatively dry formulation is a dispersion of a plurality of perforated microstructures dispersed in a suspension medium that typically comprises a hydrofluoroalkane propellant as taught in WO 9916419. The stabilized dispersions may be administered to the lung of a patient using a metered dose inhaler. Equipment useful in the commercial manufacture of spray dried medicaments are manufactured by Buchi Ltd. or Niro Corp.

At least one CARTyrin in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

### Therapeutic Applications

The present invention also provides a method for modulating or treating a disease, in a cell, tissue, organ, animal, or patient, as known in the art or as described herein, using at least one CARTyrin of the present invention, e.g., administering or contacting the cell, tissue, organ, animal, or patient with a therapeutic effective amount of CARTyrin. The present invention also provides a method for modulating or treating a disease, in a cell, tissue, organ, animal, or patient including, but not limited to, a malignant disease.

The present invention also provides a method for modulating or treating at least one malignant disease in a cell, tissue, organ, animal or patient, including, but not limited to, at least one of: leukemia, acute leukemia, acute lymphoblastic leukemia (ALL), acute lymphocytic leukemia, B-cell, T-cell or FAB ALL, acute myeloid leukemia (AML), acute myelogenous leukemia, chronic myelocytic leukemia (CML), chronic lymphocytic leukemia (CLL), hairy cell leukemia, myelodyplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, hemangioma, metastatic disease, cancer related bone resorption, cancer related bone pain, and the like.

Any method of the present invention can comprise administering an effective amount of a composition or pharmaceutical composition comprising at least one CARTyrin to a cell, tissue, organ, animal or patient in need of such modulation, treatment or therapy. Such a method can optionally further comprise co-administration or combination therapy for treating such diseases or disorders, wherein the administering of said at least one CARTyrin, specified portion or variant thereof, further comprises administering, before concurrently, and/or after, at least one selected from at least one of an alkylating agent, an a mitotic inhibitor, and a radiopharmaceutical. Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J. each of which references are entirely incorporated herein by reference.

Preferred doses can optionally include about 0.1-99 and/or 100-500 mg/kg/administration, or any range, value or fraction thereof, or to achieve a serum concentration of about 0.1-5000 μg/ml serum concentration per single or multiple administration, or any range, value or fraction thereof. A preferred dosage range for the CARTyrin of the present invention is from about 1 mg/kg, up to about 3, about 6 or about 12 mg/kg of body weight of the patient.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably, 0.1 to 10 milligrams per kilogram per administration or in sustained release form is effective to obtain desired results.

As a non-limiting example, treatment of humans or animals can be provided as a one-time or periodic dosage of at least one CARTyrin of the present invention about 0.1 to 100 mg/kg or any range, value or fraction thereof per day, on at least one of day 1-40, or, alternatively or additionally, at least one of week 1-52, or, alternatively or additionally, at least one of 1-20 years, or any combination thereof, using single, infusion or repeated doses.

Dosage forms (composition) suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

For parenteral administration, the CARTyrin can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and about 1-10% human serum albumin. Liposomes and nonaqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

### Alternative Administration

Many known and developed modes can be used according to the present invention for administering pharmaceutically effective amounts of at least one CARTyrin according to the present invention. While pulmonary administration is used in the following description, other modes of administration can be used according to the present invention with suitable results. CARTyrins of the present invention can be delivered in a carrier, as a solution, emulsion, colloid, or suspension, or as a dry powder, using any of a variety of devices and methods suitable for administration by inhalation or other modes described here within or known in the art.

### Parenteral Formulations and Administration

Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

### Alternative Delivery

The invention further relates to the administration of at least one CARTyrin by parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means. At least one CARTyrin composition can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

### Infusion of Modified Cells as Adoptive Cell Therapy

The disclosure provides modified cells that express one or more CARs and/or CARTyrins of the disclosure that have been selected and/or expanded for administration to a subject in need thereof. Modified cells of the disclosure may be formulated for storage at any temperature including room temperature and body temperature. Modified cells of the disclosure may be formulated for cryopreservation and subsequent thawing. Modified cells of the disclosure may be formulated in a pharmaceutically acceptable carrier for direct administration to a subject from sterile packaging. Modified cells of the disclosure may be formulated in a pharmaceutically acceptable carrier with an indicator of cell viability and/or CAR/CARTyrin expression level to ensure a minimal level of cell function and CAR/CARTyrin expression. Modified cells of the disclosure may be formulated in a pharmaceutically acceptable carrier at a prescribed density with one or more reagents to inhibit further expansion and/or prevent cell death.

### Inducible Proapoptotic Polypeptides

Inducible proapoptotic polypeptides of the disclosure are superior to existing inducible polypeptides because the inducible proapoptotic polypeptides of the disclosure are far less immunogenic. While inducible proapoptotic polypeptides of the disclosure are recombinant polypeptides, and, therefore, non-naturally occurring, the sequences that are recombined to produce the inducible proapoptotic polypeptides of the disclosure do not comprise non-human sequences that the host human immune system could recognize as “non-self” and, consequently, induce an immune response in the subject receiving an inducible proapoptotic polypeptide of the disclosure, a cell comprising the inducible proapoptotic polypeptide or a composition comprising the inducible proapoptotic polypeptide or the cell comprising the inducible proapoptotic polypeptide.

The disclosure provides inducible proapoptotic polypeptides comprising a ligand binding region, a linker, and a proapoptotic peptide, wherein the inducible proapoptotic polypeptide does not comprise a non-human sequence. In certain embodiments, the non-human sequence comprises a restriction site. In certain embodiments, the proapoptotic peptide is a caspase polypeptide. In certain embodiments, the caspase polypeptide is a caspase 9 polypeptide. In certain embodiments, the caspase 9 polypeptide is a truncated caspase 9 polypeptide. Inducible proapoptotic polypeptides of the disclosure may be non-naturally occurring.

Caspase polypeptides of the disclosure include, but are not limited to, caspase 1, caspase 2, caspase 3, caspase 4, caspase 5, caspase 6, caspase 7, caspase 8, caspase 9, caspase 10, caspase 11, caspase 12, and caspase 14. Caspase polypeptides of the disclosure include, but are not limited to, those caspase polypeptides associated with apoptosis including caspase 2, caspase 3, caspase 6, caspase 7, caspase 8, caspase 9, and caspase 10. Caspase polypeptides of the disclosure include, but are not limited to, those caspase polypeptides that initiate apoptosis, including caspase 2, caspase 8, caspase 9, and caspase 10. Caspase polypeptides of the disclosure include, but are not limited to, those caspase polypeptides that execute apoptosis, including caspase 3, caspase 6, and caspase 7.

Caspase polypeptides of the disclosure may be encoded by an amino acid or a nucleic acid sequence having one or more modifications compared to a wild type amino acid or a nucleic acid sequence. The nucleic acid sequence encoding a caspase polypeptide of the disclosure may be codon optimized. The one or more modifications to an amino acid and/or nucleic acid sequence of a caspase polypeptide of the disclosure may increase an interaction, a cross-linking, a cross-activation, or an activation of the caspase polypeptide of the disclosure compared to a wild type amino acid or a nucleic acid sequence. Alternatively, or in addition, the one or more modifications to an amino acid and/or nucleic acid sequence of a caspase polypeptide of the disclosure may decrease the immunogenicity of the caspase polypeptide of the disclosure compared to a wild type amino acid or a nucleic acid sequence.

Caspase polypeptides of the disclosure may be truncated compared to a wild type caspase polypeptide. For example, a caspase polypeptide may be truncated to eliminate a sequence encoding a Caspase Activation and Recruitment Domain (CARD) to eliminate or minimize the possibility of activating a local inflammatory response in addition to initiating apoptosis in the cell comprising an inducible caspase polypeptide of the disclosure. The nucleic acid sequence encoding a caspase polypeptide of the disclosure may be spliced to form a variant amino acid sequence of the caspase polypeptide of the disclosure compared to a wild type caspase polypeptide. Caspase polypeptides of the disclosure may be encoded by recombinant and/or chimeric sequences. Recombinant and/or chimeric caspase polypeptides of the disclosure may include sequences from one or more different caspase polypeptides. Alternatively, or in addition, recombinant and/or chimeric caspase polypeptides of the disclosure may include sequences from one or more species (e.g. a human sequence and a non-human sequence). Caspase polypeptides of the disclosure may be non-naturally occurring.

The ligand binding region of an inducible proapoptotic polypeptide of the disclosure may include any polypeptide sequence that facilitates or promotes the dimerization of a first inducible proapoptotic polypeptide of the disclosure with a second inducible proapoptotic polypeptide of the disclosure, the dimerization of which activates or induces cross-linking of the proapoptotic polypeptides and initiation of apoptosis in the cell.

The ligand-binding (“dimerization”) region may comprise any polypeptide or functional domain thereof that will allow for induction using an endogenous or non-naturally occurring ligand (i.e. and induction agent), for example, a non-naturally occurring synthetic ligand. The ligand-binding region may be internal or external to the cellular membrane, depending upon the nature of the inducible proapoptotic polypeptide and the choice of ligand (i.e. induction agent). A wide variety of ligand-binding polypeptides and functional domains thereof, including receptors, are known. Ligand-binding regions of the disclosure may include one or more sequences from a receptor. Of particular interest are ligand-binding regions for which ligands (for example, small organic ligands) are known or may be readily produced. These ligand-binding regions or receptors may include, but are not limited to, the FKBPs and cyclophilin receptors, the steroid receptors, the tetracycline receptor, and the like, as well as “non-naturally occurring” receptors, which can be obtained from antibodies, particularly the heavy or light chain subunit, mutated sequences thereof, random amino acid sequences obtained by stochastic procedures, combinatorial syntheses, and the like. In certain embodiments, the ligand-binding region is selected from the group consisting of a FKBP ligand-binding region, a cyclophilin receptor ligand-binding region, a steroid receptor ligand-binding region, a cyclophilin receptors ligand-binding region, and a tetracycline receptor ligand-binding region.

The ligand-binding regions comprising one or more receptor domain(s) may be at least about 50 amino acids, and fewer than about 350 amino acids, usually fewer than 200 amino acids, either as the endogenous domain or truncated active portion thereof. The binding region may, for example, be small (<25 kDa, to allow efficient transfection in viral vectors), monomeric, nonimmunogenic, have synthetically accessible, cell permeable, nontoxic ligands that can be configured for dimerization.

The ligand-binding regions comprising one or more receptor domain(s) may be intracellular or extracellular depending upon the design of the inducible proapoptotic polypeptide and the availability of an appropriate ligand (i.e. induction agent). For hydrophobic ligands, the binding region can be on either side of the membrane, but for hydrophilic ligands, particularly protein ligands, the binding region will usually be external to the cell membrane, unless there is a transport system for internalizing the ligand in a form in which it is available for binding. For an intracellular receptor, the inducible proapoptotic polypeptide or a transposon or vector comprising the inducible proapoptotic polypeptide may encode a signal peptide and transmembrane domain 5′ or 3′ of the receptor domain sequence or may have a lipid attachment signal sequence 5′ of the receptor domain sequence. Where the receptor domain is between the signal peptide and the transmembrane domain, the receptor domain will be extracellular.

Antibodies and antibody subunits, e.g., heavy or light chain, particularly fragments, more particularly all or part of the variable region, or fusions of heavy and light chain to create high-affinity binding, can be used as a ligand binding region of the disclosure. Antibodies that are contemplated include ones that are an ectopically expressed human product, such as an extracellular domain that would not trigger an immune response and generally not expressed in the periphery (i.e., outside the CNS/brain area). Such examples, include, but are not limited to low affinity nerve growth factor receptor (LNGFR), and embryonic surface proteins (i.e., carcinoembryonic antigen). Yet further, antibodies can be prepared against haptenic molecules, which are physiologically acceptable, and the individual antibody subunits screened for binding affinity. The cDNA encoding the subunits can be isolated and modified by deletion of the constant region, portions of the variable region, mutagenesis of the variable region, or the like, to obtain a binding protein domain that has the appropriate affinity for the ligand. In this way, almost any physiologically acceptable haptenic compound can be employed as the ligand or to provide an epitope for the ligand. Instead of antibody units, endogenous receptors can be employed, where the binding region or domain is known and there is a useful or known ligand for binding.

For multimerizing the receptor, the ligand for the ligand-binding region/receptor domains of the inducible proapoptotic polypeptides may be multimeric in the sense that the ligand can have at least two binding sites, with each of the binding sites capable of binding to a ligand receptor region (i.e. a ligand having a first binding site capable of binding the ligand-binding region of a first inducible proapoptotic polypeptide and a second binding site capable of binding the ligand-binding region of a second inducible proapoptotic polypeptide, wherein the ligand-binding regions of the first and the second inducible proapoptotic polypeptides are either identical or distinct). Thus, as used herein, the term “multimeric ligand binding region” refers to a ligand-binding region of an inducible proapoptotic polypeptide of the disclosure that binds to a multimeric ligand. Multimeric ligands of the disclosure include dimeric ligands. A dimeric ligand of the disclosure may have two binding sites capable of binding to the ligand receptor domain. In certain embodiments, multimeric ligands of the disclosure are a dimer or higher order oligomer, usually not greater than about tetrameric, of small synthetic organic molecules, the individual molecules typically being at least about 150 Da and less than about 5 kDa, usually less than about 3 kDa. A variety of pairs of synthetic ligands and receptors can be employed. For example, in embodiments involving endogenous receptors, dimeric FK506 can be used with an FKBP12 receptor, dimerized cyclosporin A can be used with the cyclophilin receptor, dimerized estrogen with an estrogen receptor, dimerized glucocorticoids with a glucocorticoid receptor, dimerized tetracycline with the tetracycline receptor, dimerized vitamin D with the vitamin D receptor, and the like. Alternatively higher orders of the ligands, e.g., trimeric can be used. For embodiments involving non-naturally occurring receptors, e.g., antibody subunits, modified antibody subunits, single chain antibodies comprised of heavy and light chain variable regions in tandem, separated by a flexible linker, or modified receptors, and mutated sequences thereof, and the like, any of a large variety of compounds can be used. A significant characteristic of the units comprising a multimeric ligand of the disclosure is that each binding site is able to bind the receptor with high affinity, and preferably, that they are able to be dimerized chemically. Also, methods are available to balance the hydrophobicity/hydrophilicity of the ligands so that they are able to dissolve in serum at functional levels, yet diffuse across plasma membranes for most applications.

Activation of inducible proapoptotic polypeptides of the disclosure may be accomplished through, for example, chemically induced dimerization (CID) mediated by an induction agent to produce a conditionally controlled protein or polypeptide. Proapoptotic polypeptides of the disclosure not only inducible, but the induction of these polypeptides is also reversible, due to the degradation of the labile dimerizing agent or administration of a monomeric competitive inhibitor.

In certain embodiments, the ligand binding region comprises a FK506 binding protein 12 (FKBP12) polypeptide. In certain embodiments, the ligand binding region comprises a FKBP12 polypeptide having a substitution of valine (V) for phenylalanine (F) at position 36 (F36V). In certain embodiments, in which the ligand binding region comprises a FKBP12 polypeptide having a substitution of valine (V) for phenylalanine (F) at position 36 (F36V), the induction agent may comprise AP1903, a synthetic drug (CAS Index Name: 2-Piperidinecarboxylic acid, 1-[(2S)-1-oxo-2-(3,4,5-trimethoxyphenyl)butyl]-, 1,2-ethanediylbis[imino(2-oxo-2,1-ethanediyl)oxy-3,1-phenylene[(1R)-3-(3,4-dimethoxyphenyl)propylidene]]ester, [2S-[1(R*),2R*[S*[S*[1(R*),2R*]]]]]-(9C1) CAS Registry Number: 195514-63-7; Molecular Formula: C78H98N4020; Molecular Weight: 1411.65)). In certain embodiments, in which the ligand binding region comprises a FKBP12 polypeptide having a substitution of valine (V) for phenylalanine (F) at position 36 (F36V), the induction agent may comprise AP20187 (CAS Registry Number: 195514-80-8 and Molecular Formula: C82H107N5020). In certain embodiments, the induction agent is an AP20187 analog, such as, for example, AP1510. As used herein, the induction agents AP20187, AP1903 and AP1510 may be used interchangeably.

AP1903 API is manufactured by Alphora Research Inc. and AP1903 Drug Product for Injection is made by Formatech Inc. It is formulated as a 5 mg/mL solution of AP1903 in a 25% solution of the non-ionic solubilizer Solutol HS 15 (250 mg/mL, BASF). At room temperature, this formulation is a clear, slightly yellow solution. Upon refrigeration, this formulation undergoes a reversible phase transition, resulting in a milky solution. This phase transition is reversed upon re-warming to room temperature. The fill is 2.33 mL in a 3 mL glass vial (approximately 10 mg AP1903 for Injection total per vial). Upon determining a need to administer AP1903, patients may be, for example, administered a single fixed dose of AP1903 for Injection (0.4 mg/kg) via IV infusion over 2 hours, using a non-DEHP, non-ethylene oxide sterilized infusion set. The dose of AP1903 is calculated individually for all patients, and is not be recalculated unless body weight fluctuates by 10%. The calculated dose is diluted in 100 mL in 0.9% normal saline before infusion. In a previous Phase I study of AP1903, 24 healthy volunteers were treated with single doses of AP1903 for Injection at dose levels of 0.01, 0.05, 0.1, 0.5 and 1.0 mg/kg infused IV over 2 hours. AP1903 plasma levels were directly proportional to dose, with mean Cmax values ranging from approximately 10-1275 ng/mL over the 0.01-1.0 mg/kg dose range. Following the initial infusion period, blood concentrations demonstrated a rapid distribution phase, with plasma levels reduced to approximately 18, 7, and 1% of maximal concentration at 0.5, 2 and 10 hours post-dose, respectively. AP1903 for Injection was shown to be safe and well tolerated at all dose levels and demonstrated a favorable pharmacokinetic profile. luliucci J D, et al., J Clin Pharmacol. 41: 870-9, 2001.

The fixed dose of AP1903 for injection used, for example, may be 0.4 mg/kg intravenously infused over 2 hours. The amount of AP1903 needed in vitro for effective signaling of cells is 10-100 nM (1600 Da MW). This equates to 16-160 μg/L or 0.016-1.6 μg/kg (1.6-160 μg/kg). Doses up to 1 mg/kg were well-tolerated in the Phase I study of AP1903 described above. Therefore, 0.4 mg/kg may be a safe and effective dose of AP1903 for this Phase I study in combination with the therapeutic cells.

The amino acid and/or nucleic acid sequence encoding ligand binding of the disclosure may contain sequence one or more modifications compared to a wild type amino acid or nucleic acid sequence. For example, the amino acid and/or nucleic acid sequence encoding ligand binding region of the disclosure may be a codon-optimized sequence. The one or more modifications may increase the binding affinity of a ligand (e.g. an induction agent) for the ligand binding region of the disclosure compared to a wild type polypeptide. Alternatively, or in addition, the one or more modifications may decrease the immunogenicity of the ligand binding region of the disclosure compared to a wild type polypeptide. Ligand binding regions of the disclosure and/or induction agents of the disclosure may be non-naturally occurring.

Inducible proapoptotic polypeptides of the disclosure comprise a ligand binding region, a linker and a proapoptotic peptide, wherein the inducible proapoptotic polypeptide does not comprise a non-human sequence. In certain embodiments, the non-human sequence comprises a restriction site. The linker may comprise any organic or inorganic material that permits, upon dimerization of the ligand binding region, interaction, cross-linking, cross-activation, or activation of the proapoptotic polypeptides such that the interaction or activation of the proapoptotic polypeptides initiates apoptosis in the cell. In certain embodiments, the linker is a polypeptide. In certain embodiments, the linker is a polypeptide comprising a G/S rich amino acid sequence (a “GS” linker). In certain embodiments, the linker is a polypeptide comprising the amino acid sequence GGGGS (SEQ ID NO: 18028). In preferred embodiments, the linker is a polypeptide and the nucleic acid encoding the polypeptide does not contain a restriction site for a restriction endonuclease. Linkers of the disclosure may be non-naturally occurring.

Inducible proapoptotic polypeptides of the disclosure may be expressed in a cell under the transcriptional regulation of any promoter capable of initiating and/or regulating the expression of an inducible proapoptotic polypeptide of the disclosure in that cell. The term “promoter” as used herein refers to a promoter that acts as the initial binding site for RNA polymerase to transcribe a gene. For example, inducible proapoptotic polypeptides of the disclosure may be expressed in a mammalian cell under the transcriptional regulation of any promoter capable of initiating and/or regulating the expression of an inducible proapoptotic polypeptide of the disclosure in a mammalian cell, including, but not limited to native, endogenous, exogenous, and heterologous promoters. Preferred mammalian cells include human cells. Thus, inducible proapoptotic polypeptides of the disclosure may be expressed in a human cell under the transcriptional regulation of any promoter capable of initiating and/or regulating the expression of an inducible proapoptotic polypeptide of the disclosure in a human cell, including, but not limited to, a human promoter or a viral promoter. Exemplary promoters for expression in human cells include, but are not limited to, a human cytomegalovirus (CMV) immediate early gene promoter, a SV40 early promoter, a Rous sarcoma virus long terminal repeat, β-actin promoter, a rat insulin promoter and a glyceraldehyde-3-phosphate dehydrogenase promoter, each of which may be used to obtain high-level expression of an inducible proapoptotic polypeptide of the disclosure. The use of other viral or mammalian cellular or bacterial phage promoters which are well known in the art to achieve expression of an inducible proapoptotic polypeptide of the disclosure is contemplated as well, provided that the levels of expression are sufficient for initiating apoptosis in a cell. By employing a promoter with well-known properties, the level and pattern of expression of the protein of interest following transfection or transformation can be optimized.

Selection of a promoter that is regulated in response to specific physiologic or synthetic signals can permit inducible expression of the inducible proapoptotic polypeptide of the disclosure. The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of a transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter, which drives expression of the gene of interest, is on another plasmid. Engineering of this type of system into a vector of interest may therefore be useful. Another inducible system that may be useful is the Tet-Off™ or Tet-On™ system (Clontech, Palo Alto, Calif.) originally developed by Gossen and Bujard (Gossen and Bujard, Proc. Natl. Acad. Sci. USA, 89:5547-5551, 1992; Gossen et al., Science, 268:1766-1769, 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli: the tetracycline operator sequence (to which the tetracycline repressor binds) and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tetracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system may be used so that the producer cells could be grown in the presence of tetracycline or doxycycline and prevent expression of a potentially toxic transgene, but when the vector is introduced to the patient, the gene expression would be constitutively on.

In some circumstances, it is desirable to regulate expression of a transgene in a gene therapy vector. For example, different viral promoters with varying strengths of activity are utilized depending on the level of expression desired. In mammalian cells, the CMV immediate early promoter is often used to provide strong transcriptional activation. The CMV promoter is reviewed in Donnelly, J. J., et al., 1997. Annu. Rev. Immunol. 15:617-48. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that are used depending on the desired effect include SV40, RSV LTR, HIV-1 and HIV-2 LTR, adenovirus promoters such as from the E1A, E2A, or MLP region, AAV LTR, HSV-TK, and avian sarcoma virus.

In other examples, promoters may be selected that are developmentally regulated and are active in particular differentiated cells. Thus, for example, a promoter may not be active in a pluripotent stem cell, but, for example, where the pluripotent stem cell differentiates into a more mature cell, the promoter may then be activated.

Similarly tissue specific promoters are used to effect transcription in specific tissues or cells so as to reduce potential toxicity or undesirable effects to non-targeted tissues. These promoters may result in reduced expression compared to a stronger promoter such as the CMV promoter, but may also result in more limited expression, and immunogenicity (Bojak, A., et al., 2002. Vaccine. 20:1975-79; Cazeaux, N., et al., 2002. Vaccine 20:3322-31). For example, tissue specific promoters such as the PSA associated promoter or prostate-specific glandular kallikrein, or the muscle creatine kinase gene may be used where appropriate.

Examples of tissue specific or differentiation specific promoters include, but are not limited to, the following: B29 (B cells); CD14 (monocytic cells); CD43 (leukocytes and platelets); CD45 (hematopoietic cells); CD68 (macrophages); desmin (muscle); elastase-1 (pancreatic acinar cells); endoglin (endothelial cells); fibronectin (differentiating cells, healing tissues); and Flt-1 (endothelial cells); GFAP (astrocytes).

In certain indications, it is desirable to activate transcription at specific times after administration of the gene therapy vector. This is done with such promoters as those that are hormone or cytokine regulatable. Cytokine and inflammatory protein responsive promoters that can be used include K and T kininogen (Kageyama et al., (1987) J. Biol. Chem., 262, 2345-2351), c-fos, TNF-alpha, C-reactive protein (Arcone, et al., (1988) Nucl. Acids Res., 16(8), 3195-3207), haptoglobin (Oliviero et al., (1987) EMBO J., 6, 1905-1912), serum amyloid A2, C/EBP alpha, IL-1, IL-6 (Poli and Cortese, (1989) Proc. Nat'l Acad. Sci. USA, 86, 8202-8206), Complement C3 (Wilson et al., (1990) Mol. Cell. Biol., 6181-6191), IL-8, alpha-1 acid glycoprotein (Prowse and Baumann, (1988) Mol Cell Biol, 8, 42-51), alpha-1 antitrypsin, lipoprotein lipase (Zechner et al., Mol. Cell. Biol., 2394-2401, 1988), angiotensinogen (Ron, et al., (1991) Mol. Cell. Biol., 2887-2895), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 anti-chymotrypsin. Other promoters include, for example, SV40, MMTV, Human Immunodeficiency Virus (MV), Moloney virus, ALV, Epstein Barr virus, Rous Sarcoma virus, human actin, myosin, hemoglobin, and creatine.

It is envisioned that any of the above promoters alone or in combination with another can be useful depending on the action desired. Promoters, and other regulatory elements, are selected such that they are functional in the desired cells or tissue. In addition, this list of promoters should not be construed to be exhaustive or limiting; other promoters that are used in conjunction with the promoters and methods disclosed herein.

### Armored T-Cells “Knock-Down” Strategy

T-cells of the disclosure may be genetically modified to enhance their therapeutic potential. Alternatively, or in addition, T-cells of the disclosure may be modified to render them less sensitive to immunologic and/or metabolic checkpoints. Modifications of this type “armor” the T cells of the disclosure, which, following the modification, may be referred to here as “armored” T cells. Armored T cells of the disclosure may be produced by, for example, blocking and/or diluting specific endogenous checkpoint signals delivered to the T-cells (i.e. checkpoint inhibition) within the tumor immunosuppressive microenvironment, for example.

In some embodiments, an armored T-cell of the disclosure is derived from a T cell, a NK cell, a hematopoietic progenitor cell, a peripheral blood (PB) derived T cell (including a T cell isolated or derived from G-CSF-mobilized peripheral blood), or an umbilical cord blood (UCB) derived T cell. In some embodiments, an armored T-cell of the disclosure comprises one or more of a chimeric ligand receptor (CLR comprising a protein scaffold, an antibody, an ScFv, or an antibody mimetic)/chimeric antigen receptor (CAR comprising a protein scaffold, an antibody, an ScFv, or an antibody mimetic), a CARTyrin (a CAR comprising a Centyrin), and/or a VCAR (a CAR comprising a camelid VHH or a single domain VH) of the disclosure. In some embodiments, an armored T-cell of the disclosure comprises an inducible proapoptotic polypeptide comprising (a) a ligand binding region, (b) a linker, and (c) a truncated caspase 9 polypeptide, wherein the inducible proapoptotic polypeptide does not comprise a non-human sequence. In some embodiments, the non-human sequence is a restriction site. In some embodiments, the ligand binding region inducible caspase polypeptide comprises a FK506 binding protein 12 (FKBP12) polypeptide. In some embodiments, the amino acid sequence of the FK506 binding protein 12 (FKBP12) polypeptide comprises a modification at position 36 of the sequence. In some embodiments, the modification is a substitution of valine (V) for phenylalanine (F) at position 36 (F36V). In some embodiments, an armored T-cell of the disclosure comprises an exogenous sequence. In some embodiments, the exogenous sequence comprises a sequence encoding a therapeutic protein. Exemplary therapeutic proteins may be nuclear, cytoplasmic, intracellular, transmembrane, cell-surface bound, or secreted proteins. Exemplary therapeutic proteins expressed by the armored T cell may modify an activity of the armored T cell or may modify an activity of a second cell. In some embodiments, an armored T-cell of the disclosure comprises a selection gene or a selection marker. In some embodiments, an armored T-cell of the disclosure comprises a synthetic gene expression cassette (also referred to herein as an inducible transgene construct).

In some embodiments, a T-cell of the disclosure is modified to silence or reduce expression one or more gene(s) encoding receptor(s) of inhibitory checkpoint signals to produce an armored T-cell of the disclosure. Examples of inhibitory checkpoint signals include, but are not limited to, a PD-L1 ligand binding to a PD-1 receptor on a CAR-T cell of the disclosure or a TGFβ cytokine binding to a TGFβRII receptor on a CAR-T cell. Receptors of inhibitory checkpoint signals are expressed on the cell surface or within the cytoplasm of a T-cell. Silencing or reducing expressing of the gene encoding the receptor of the inhibitory checkpoint signal results a loss of protein expression of the inhibitory checkpoint receptors on the surface or within the cytoplasm of an armored T-cell of the disclosure. Thus, armored T cells of the disclosure having silenced or reduced expression of one or more genes encoding an inhibitory checkpoint receptor is resistant, non-receptive or insensitive to checkpoint signals. The armored T cell's resistance or decreased sensitivity to inhibitory checkpoint signals enhances the armored T cell's therapeutic potential in the presence of these inhibitory checkpoint signals. Inhibitory checkpoint signals include but are not limited to the examples listed in Table 1. Exemplary inhibitory checkpoint signals that may be silenced in an armored T cell of the disclosure include, but are not limited to, PD-1 and TGFβRII.

TABLE 1
Exemplary Inhibitory Checkpoint Signals (and proteins that
induce immunosuppression). A CSR of the disclosure may comprise
an endodomain of any one of the proteins of this table.

| Full Name | Abbreviation | SEQ ID NO: |
|---|---|---|
| Programmed cell death protein 1 | PD1 | 14643-14644 |
| transforming growth factor β Receptor 1 | TGFβR1 | 14645 |
| transforming growth factor β Receptor 2 | TGFβR2 | 14646 |
| T-cell immunoglobulin and mucin-domain | TIM3 | 14647 |
| containing-3 | | |
| Lymphocyte-activation gene 3 | LAG3 | 14648 |
| Cytotoxic T-lymphocyte protein 4 | CTLA4 | 14649 |
| B- and T-lymphocyte attenuator | BTLA | 14650 |
| Killer cell immunoglobulin-like receptor | KIR | 14651 |
| Alpha-2A adrenergic receptor | A2aR | 14652 |
| V-type immunoglobulin domain-containing | VISTA | 14653 |
| suppressor of T-cell activation | | |
| T-cell immunoreceptor with Ig and ITIM | TIGIT | 14654 |
| domains | | |
| Programmed cell death 1 ligand 1 | B7H1 or PD-L1 | 14655 |
| Programmed cell death 1 ligand 2 | B7DC or PD-L2 | 14656 |
| T-lymphocyte activation antigen CD80 | B7-1 or CD80 | 14657 |
| T-lymphocyte activation antigen CD86 | B7-2 or CD86 | 14658 |
| CD160 antigen | CD160 | 14659 |
| Leukocyte-associated immunoglobulin-like | LAIR1 | 14660 |
| receptor 1 | | |
| T-cell immunoglobulin and mucin domain- | TIM4 or TIMD4 | 14661 |
| containing protein 4 | | |
| Natural killer cell receptor 2B4 | 2B4 or CD244 | 14662 |
| Major Histocompatibility Complex type I | MHC I | 14663 |
| Major Histocompatibility Complex type II | MHC II | |
| Putative 2-methylcitrate dehydratase receptor | PDH1R | |
| T-cell immunoglobulin and mucin domain 1 | TIM1R | |
| receptor | | |
| T-cell immunoglobulin and mucin domain 4 | TIM4R | |
| receptor | | |
| B7-H3 receptor | B7H3R or CD176 | |
| | Receptor | |
| B7-H4 receptor | B7H4R | |
| Immunoglobulin-like transcript (ILT) 3 receptor | ILT3R | |
| phosphoinositide 3-kinase, subunit alpha | PI3K alpha | 14664 |
| phosphoinositide 3-kinase, subunit gamma | PI3K gamma | 14665 |
| Tyrosine-protein phosphatase non-receptor type | SHP2 or PTPN11 | 14666 |
| 11 | | |
| Protein phosphatase 2, subunit gamma | PP2A gamma | 14667 |
| Protein phosphatase 2, subunit beta | PP2A beta | 14668 |
| Protein phosphatase 2, subunit delta | PP2A delta | 14669 |
| Protein phosphatase 2, subunit epsilon | PP2A epsilon | 14670 |
| Protein phosphatase 2, subunit alpha | PP2A alpha | 14671 |
| T-cell Receptor, subunit alpha | TCR alpha | 14672 |
| T-cell Receptor, subunit beta | TCR beta | 14673 |
| T-cell Receptor, subunit zeta | TCR zeta | 14674 |
| T-cell Receptor, subunit CD3 epsilon | TCR CD3 epsilon | 14675 |
| T-cell Receptor, subunit CD3 gamma | TCR CD3 gamma | 14676 |
| T-cell Receptor, subunit CD3 delta | TCR CD3 delta | 14677 |
| Cluster of Differentiation 28 | CD28 | 14678 |
| Galectins | Galectins | |
| Galectin 9 | Galectin 9 | 14679 |
| High Mobility Group Box 1 | HMGB1 | 14680 |
| Arginase 1 | ARG1 | 14681 |
| Prostaglandin-Endoperoxide Synthase 1 | PTGS1 | 14682 |
| Prostaglandin-Endoperoxide Synthase 2 | PTGS2 | 14683 |
| Mucin 1, Cell Surface Associated | MUC1 | 14684 |
| Mucin 2, Oligomeric Mucus/Gel-Forming | MUC2 | 14685 |
| Mucin 3A, Cell Surface Associated | MUC3A | 14686 |
| Mucin 3B, Cell Surface Associated | MUC3B | 14687 |
| Mucin 4, Cell Surface Associated | MUC4 | 14688 |
| Mucin 5AC, Oligomeric Mucus/Gel-Forming | MUC5AC | 14689 |
| Mucin 5B, Oligomeric Mucus/Gel-Forming | MUC5B | 14690 |
| Mucin 6, Oligomeric Mucus/Gel-Forming | MUC6 | 14691 |
| Mucin 7, Secreted | MUC7 | 14692 |
| Mucin 8 | MUC8 | |
| Mucin 12, Cell Surface Associated | MUC12 | 14693 |
| Mucin 13, Cell Surface Associated | MUC13 | 14694 |
| Mucin 15, Cell Surface Associated | MUC15 | 14695 |
| Mucin 16, Cell Surface Associated | MUC16 | 14696 |
| Mucin 17, Cell Surface Associated | MUC17 | 14697 |
| Mucin 19, Oligomeric | MUC19 | 14698 |
| Mucin 20, Cell Surface Associated | MUC20 | 14699 |
| Mucin 21, Cell Surface Associated | MUC21 | 14700 |
| Mucin 22 | MUC22 | 14701 |
| Indoleamine 2,3-Dioxygenase 1 | IDO1 | 14702 |
| Indoleamine 2,3-Dioxygenase 2 | IDO2 | 14703 |
| Inducible T Cell Costimulator Ligand | ICOSLG | 14704 |
| ROS Proto-Oncogene 1, Receptor Tyrosine | ROS1 | 14705 |
| Kinase | | |
| Tumor Necrosis Factor Receptor Superfamily | 4-1BB, CD137, ILA or | 14706 |
| Member 9 | TNFRSF9 | |
| 4-1BB Ligand | 4-1BB-L | 14707 |
| Glucocorticoid-induced TNFR family related | GITR | 14708 |
| gene | | |
| Glucocorticoid-induced TNFR family related | GITRL | 14709 |
| gene ligand | | |

In some embodiments, a T-cell of the disclosure is modified to silence or reduce expression of one or more gene(s) encoding intracellular proteins involved in checkpoint signaling to produce an armored T-cell of the disclosure. The activity of a T-cell of the disclosure may be enhanced by targeting any intracellular signaling protein involved in a checkpoint signaling pathway, thereby achieving checkpoint inhibition or interference to one or more checkpoint pathways. Intracellular signaling proteins involved in checkpoint signaling include, but are not limited to, exemplary intracellular signaling proteins listed in Table 2.

TABLE 2
Exemplary Intracellular Signaling Proteins.

| Full Name | Abbreviation | SEQ ID NO: |
|---|---|---|
| phosphoinositide 3-kinase, subunit alpha | PI3K alpha | 14710 |
| phosphoinositide 3-kinase, subunit gamma | PI3K gamma | 14711 |
| Tyrosine-protein phosphatase non-receptor type | SHP2 or PTPN11 | 14712 |
| 11 | | |
| Protein phosphatase 2, subunit gamma | PP2A gamma | 14713 |
| Protein phosphatase 2, subunit beta | PP2A beta | 14714 |
| Protein phosphatase 2, subunit delta | PP2A delta | 14715 |
| Protein phosphatase 2, subunit epsilon | PP2A epsilon | 14716 |
| Protein phosphatase 2, subunit alpha | PP2A alpha | 14717 |
| RAC-alpha serine/threonine-protein kinase | AKT or PKB | 14718 |
| Tyrosine-protein kinase ZAP-70 | ZAP70 | 14719 |
| Amino acid sequence (KIEELE)-containing | KIEELE-domain | |
| domain protein | containing proteins | |
| BCL2 associated athanogene 6 | Bat3, Bag6 or Scythe | 14720 |
| B-cell lymphoma-extra large | Bcl-xL | 14721 |
| Bcl-2-related protein A1 | Bfl-1 or BCL2A1 | 14722 |

In some embodiments, a T-cell of the disclosure is modified to silence or reduce expression of one or more gene(s) encoding a transcription factor that hinders the efficacy of a therapy to produce an armored T-cell of the disclosure. The activity of armored T-cells may be enhanced or modulated by silencing or reducing expression (or repressing a function) of a transcription factor that hinders the efficacy of a therapy. Exemplary transcription factors that may be modified to silence or reduce expression or to repress a function thereof include, but are not limited to, the exemplary transcription factors listed in Table 3. For example, expression of a FOXP3 gene may be silenced or reduced in an armored T cell of the disclosure to prevent or reduce the formation of T regulatory CAR-T-cells (CAR-Treg cells), the expression or activity of which may reduce efficacy of a therapy.

TABLE 3
Exemplary Transcription Factors.

| Full Name | Abbreviation | SEQ ID NO: |
|---|---|---|
| activity-dependent neuroprotector homeobox | ADNP | 14723 |
| ADNP homeobox 2 | ADNP2 | 14724 |
| AE binding protein 1 | AEBP1 | 14725 |
| AE binding protein 2 | AEBP2 | 14726 |
| AF4/FMR2 family member 1 | AFF1 | 14727 |
| AF4/FMR2 family member 2 | AFF2 | 14728 |
| AF4/FMR2 family member 3 | AFF3 | 14729 |
| AF4/FMR2 family member 4 | AFF4 | 14730 |
| AT-hook containing transcription factor 1 | AHCTF1 | 14731 |
| aryl hydrocarbon receptor | AHR | 14732 |
| aryl-hydrocarbon receptor repressor | AHRR | 14733 |
| autoimmune regulator | AIRE | 14734 |
| AT-hook transcription factor | AKNA | 14735 |
| ALX homeobox 1 | ALX1 | 14736 |
| ALX homeobox 3 | ALX3 | 14737 |
| ALX homeobox 4 | ALX4 | 14738 |
| ankyrin repeat and zinc finger domain containing 1 | ANKZF1 | 14739 |
| adaptor related protein complex 5 zeta 1 subunit | AP5Z1 | 14740 |
| androgen receptor | AR | 14741 |
| arginine-fifty homeobox | ARGFX | 14742 |
| Rho GTPase activating protein 35 | ARHGAP35 | 14743 |
| AT-rich interaction domain 1A | ARID1A | 14744 |
| AT-rich interaction domain 1B | ARID1B | 14745 |
| AT-rich interaction domain 2 | ARID2 | 14746 |
| AT-rich interaction domain 3A | ARID3A | 14747 |
| AT-rich interaction domain 3B | ARID3B | 14748 |
| AT-rich interaction domain 3C | ARID3C | 14749 |
| AT-rich interaction domain 4A | ARID4A | 14750 |
| AT-rich interaction domain 4B | ARID4B | 14751 |
| AT-rich interaction domain 5A | ARID5A | 14752 |
| AT-rich interaction domain 5B | ARID5B | 14753 |
| aryl hydrocarbon receptor nuclear translocator | ARNT | 14754 |
| aryl hydrocarbon receptor nuclear translocator 2 | ARNT2 | 14755 |
| aryl hydrocarbon receptor nuclear translocator like | ARNTL | 14756 |
| aryl hydrocarbon receptor nuclear translocator like 2 | ARNTL2 | 14757 |
| aristaless related homeobox | ARX | 14758 |
| achaete-scute family bHLH transcription factor 1 | ASCL1 | 14759 |
| achaete-scute family bHLH transcription factor 2 | ASCL2 | 14760 |
| achaete-scute family bHLH transcription factor 3 | ASCL3 | 14761 |
| achaete-scute family bHLH transcription factor 4 | ASCL4 | 14762 |
| achaete-scute family bHLH transcription factor 5 | ASCL5 | 14763 |
| ash1 (absent, small, or homeotic)-like (Drosophila) | ASH1L | 14764 |
| ash2 (absent, small, or homeotic)-like (Drosophila) | ASH2L | 14765 |
| activating transcription factor 1 | ATF1 | 14766 |
| activating transcription factor 2 | ATF2 | 14767 |
| activating transcription factor 3 | ATF3 | 14768 |
| activating transcription factor 4 | ATF4 | 14769 |
| activating transcription factor 5 | ATF5 | 14770 |
| activating transcription factor 6 | ATF6 | 14771 |
| activating transcription factor 6 beta | ATF6B | 14772 |
| activating transcription factor 7 | ATF7 | 14773 |
| atonal bHLH transcription factor 1 | ATOH1 | 14774 |
| atonal bHLH transcription factor 7 | ATOH7 | 14775 |
| atonal bHLH transcription factor 8 | ATOH8 | 14776 |
| alpha thalassemia/mental retardation syndrome X- | ATRX | 14777 |
| linked | | |
| ataxin 7 | ATXN7 | 14778 |
| BTB and CNC homology 1, basic leucine zipper | BACH1 | 14779-14780 |
| transcription factor 1 | | |
| BTB domain and CNC homolog 2 | BACH2 | 14781 |
| BarH like homeobox 1 | BARHL1 | 14782 |
| BarH like homeobox 2 | BARHL2 | 14783 |
| BARX homeobox 1 | BARX1 | 14784 |
| BARX homeobox 2 | BARX2 | 14785 |
| Basic Leucine Zipper ATF-Like Transcription Factor, | Batf | 14786 |
| basic leucine zipper transcription factor, ATF-like | BATF | 14786 |
| basic leucine zipper transcription factor, ATF-like 2 | BATF2 | 14787 |
| basic leucine zipper transcription factor, ATF-like 3 | BATF3 | 14788 |
| bobby sox homolog (Drosophila) | BBX | 14789 |
| B-cell CLL/lymphoma 11A | BCL11A | 14790 |
| B-cell CLL/lymphoma 11B | BCL11B | 14791 |
| B-cell CLL/lymphoma 3 | BCL3 | 14792 |
| B-cell CLL/lymphoma 6 | BCL6 | 14793 |
| B-cell CLL/lymphoma 6, member B | BCL6B | 14794 |
| BCL2 associated transcription factor 1 | BCLAF1 | 14795 |
| basic helix-loop-helix family member a15 | BHLHA15 | 14796 |
| basic helix-loop-helix family member a9 | BHLHA9 | 14797 |
| basic helix-loop-helix domain containing, class B, 9 | BHLHB9 | 14798 |
| basic helix-loop-helix family member e22 | BHLHE22 | 14799 |
| basic helix-loop-helix family member e23 | BHLHE23 | 14800 |
| basic helix-loop-helix family member e40 | BHLHE40 | 14801 |
| basic helix-loop-helix family member e41 | BHLHE41 | 14802 |
| Beta-Interferon Gene Positive-Regulatory Domain I | Blimp-1 | 14803 |
| Binding Factor | | |
| bone morphogenetic protein 2 | BMP2 | 14804 |
| basonuclin 1 | BNC1 | 14805 |
| basonuclin 2 | BNC2 | 14806 |
| bolA family member 1 | BOLA1 | 14807 |
| bolA family member 2 | BOLA2 | 14808 |
| bolA family member 3 | BOLA3 | 14809 |
| bromodomain PHD finger transcription factor | BPTF | 14810 |
| breast cancer 1 | BRCA1 | 14811 |
| brain specific homeobox | BSX | 14812 |
| chromosome 20 open reading frame 194 | C20orf194 | 14813 |
| calmodulin binding transcription activator 1 | CAMTA1 | 14814 |
| calmodulin binding transcription activator 2 | CAMTA2 | 14815 |
| calcium regulated heat stable protein 1 | CARHSP1 | 14816 |
| castor zinc finger 1 | CASZ1 | 14817 |
| core-binding factor, beta subunit | CBFB | 14818 |
| coiled-coil domain containing 79 | CCDC79 | 14819 |
| cell division cycle 5 like | CDC5L | 14820 |
| caudal type homeobox 1 | CDX1 | 14821 |
| caudal type homeobox 2 | CDX2 | 14822 |
| caudal type homeobox 4 | CDX4 | 14823 |
| CCAAT/enhancer binding protein alpha | CEBPA | 14824 |
| CCAAT/enhancer binding protein beta | CEBPB | 14825 |
| CCAAT/enhancer binding protein delta | CEBPD | 14826 |
| CCAAT/enhancer binding protein epsilon | CEBPE | 14827 |
| CCAAT/enhancer binding protein gamma | CEBPG | 14828 |
| CCAAT/enhancer binding protein zeta | CEBPZ | 14829 |
| centromere protein T | CENPT | 14830 |
| ceramide synthase 3 | CERS3 | 14831 |
| ceramide synthase 6 | CERS6 | 14832 |
| chromosome alignment maintaining phosphoprotein 1 | CHAMP1 | 14833 |
| capicua transcriptional repressor | CIC | 14834 |
| CDKN1A interacting zinc finger protein 1 | CIZ1 | 14835 |
| clock circadian regulator | CLOCK | 14836 |
| CCR4-NOT transcription complex subunit 4 | CNOT4 | 14837 |
| CPX chromosome region, candidate 1 | CPXCR1 | 14838 |
| cramped chromatin regulator homolog 1 | CRAMP1 | 14839 |
| cAMP responsive element binding protein 1 | CREB1 | 14840 |
| cAMP responsive element binding protein 3 | CREB3 | 14841 |
| cAMP responsive element binding protein 3-like 1 | CREB3L1 | 14842 |
| cAMP responsive element binding protein 3-like 2 | CREB3L2 | 14843 |
| cAMP responsive element binding protein 3-like 3 | CREB3L3 | 14844 |
| cAMP responsive element binding protein 3-like 4 | CREB3L4 | 14845 |
| cAMP responsive element binding protein 5 | CREB5 | 14846 |
| CREB binding protein | CREBBP | 14847 |
| cAMP responsive element binding protein-like 2 | CREBL2 | 14848 |
| CREB3 regulatory factor | CREBRF | 14849 |
| CREB/ATF bZIP transcription factor | CREBZF | 14850 |
| cAMP responsive element modulator | CREM | 14851 |
| cone-rod homeobox | CRX | 14852 |
| cysteine-serine-rich nuclear protein 1 | CSRNP1 | 14853 |
| cysteine-serine-rich nuclear protein 2 | CSRNP2 | 14854 |
| cysteine-serine-rich nuclear protein 3 | CSRNP3 | 14855 |
| CCCTC-binding factor (zinc finger protein) | CTCF | 14856 |
| CCCTC-binding factor like | CTCFL | 14857 |
| cut-like homeobox 1 | CUX1 | 14858-14859 |
| cut-like homeobox 2 | CUX2 | 14860 |
| CXXC finger protein 1 | CXXC1 | 14861 |
| dachshund family transcription factor 1 | DACH1 | 14862 |
| dachshund family transcription factor 2 | DACH2 | 14863 |
| D site of albumin promoter (albumin D-box) binding | DBP | 14864 |
| protein | | |
| developing brain homeobox 1 | DBX1 | 14865 |
| developing brain homeobox 2 | DBX2 | 14866 |
| damage specific DNA binding protein 2 | DDB2 | 14867 |
| DNA damage inducible transcript 3 | DDIT3 | 14868 |
| DEAF1, transcription factor | DEAF1 | 14869 |
| distal-less homeobox 1 | DLX1 | 14870 |
| distal-less homeobox 2 | DLX2 | 14871 |
| distal-less homeobox 3 | DLX3 | 14872 |
| distal-less homeobox 4 | DLX4 | 14873 |
| distal-less homeobox 5 | DLX5 | 14874 |
| distal-less homeobox 6 | DLX6 | 14875 |
| DNA methyltransferase 1 associated protein 1 | DMAP1 | 14876 |
| diencephalon/mesencephalon homeobox 1 | DMBX1 | 14877 |
| doublesex and mab-3 related transcription factor 1 | DMRT1 | 14878 |
| doublesex and mab-3 related transcription factor 2 | DMRT2 | 14879 |
| doublesex and mab-3 related transcription factor 3 | DMRT3 | 14880 |
| DMRT like family Al | DMRTA1 | 14881 |
| DMRT like family A2 | DMRTA2 | 14882 |
| DMRT like family B with proline rich C-terminal 1 | DMRTB1 | 14883 |
| DMRT like family C1 | DMRTC1 | 14884 |
| DMRT like family C1B | DMRTC1B | 14884 |
| DMRT like family C2 | DMRTC2 | 14885 |
| cyclin D binding myb like transcription factor 1 | DMTF1 | 14886 |
| DnaJ heat shock protein family (Hsp40) member C1 | DNAJC1 | 14887 |
| DnaJ heat shock protein family (Hsp40) member C2 | DNAJC2 | 14888 |
| DnaJ heat shock protein family (Hsp40) member C21 | DNAJC21 | 14889 |
| DNA (cytosine-5-)-methyltransferase 1 | DNMT1 | 14890 |
| DNA (cytosine-5-)-methyltransferase 3 alpha | DNMT3A | 14891 |
| DNA (cytosine-5-)-methyltransferase 3 beta | DNMT3B | 14892 |
| DNA (cytosine-5-)-methyltransferase 3-like | DNMT3L | 14893 |
| double PHD fingers 1 | DPF1 | 14894 |
| double PHD fingers 2 | DPF2 | 14895 |
| double PHD fingers 3 | DPF3 | 14896 |
| divergent-paired related homeobox | DPRX | 14897 |
| down-regulator of transcription 1 | DR1 | 14898 |
| DR1 associated protein 1 | DRAP1 | 14899 |
| dorsal root ganglia homeobox | DRGX | 14900 |
| double homeobox 4 | DUX4 | 14901 |
| double homeobox 4 like 9 | DUX4L9 | 14902 |
| double homeobox A | DUXA | 14903 |
| E2F transcription factor 1 | E2F1 | 14904 |
| E2F transcription factor 2 | E2F2 | 14905 |
| E2F transcription factor 3 | E2F3 | 14906 |
| E2F transcription factor 4 | E2F4 | 14907 |
| E2F transcription factor 5 | E2F5 | 14908 |
| E2F transcription factor 6 | E2F6 | 14909 |
| E2F transcription factor 7 | E2F7 | 14910 |
| E2F transcription factor 8 | E2F8 | 14911 |
| E4F transcription factor 1 | E4F1 | 14912 |
| early B-cell factor 1 | EBF1 | 14913 |
| early B-cell factor 2 | EBF2 | 14914 |
| early B-cell factor 3 | EBF3 | 14915 |
| early B-cell factor 4 | EBF4 | 14916 |
| early growth response 1 | EGR1 | 14917 |
| early growth response 2 | EGR2 | 14918 |
| early growth response 3 | EGR3 | 14919 |
| early growth response 4 | EGR4 | 14920 |
| ets homologous factor | EHF | 14921 |
| E74-like factor 1 (ets domain transcription factor) | ELF1 | 14922 |
| E74-like factor 2 (ets domain transcription factor) | ELF2 | 14923 |
| E74-like factor 3 (ets domain transcription factor, | ELF3 | 14924 |
| epithelial-specific) | | |
| E74-like factor 4 (ets domain transcription factor) | ELF4 | 14925 |
| E74-like factor 5 (ets domain transcription factor) | ELF5 | 14926 |
| ELK1, member of ETS oncogene family | ELK1 | 14927 |
| ELK3, ETS-domain protein (SRF accessory protein 2) | ELK3 | 14928 |
| ELK4, ETS-domain protein (SRF accessory protein 1) | ELKA | 14929 |
| ELM2 and Myb/SANT-like domain containing 1 | ELMSAN1 | 14930 |
| empty spiracles homeobox 1 | EMX1 | 14931 |
| empty spiracles homeobox 2 | EMX2 | 14932 |
| engrailed homeobox 1 | EN1 | 14933 |
| engrailed homeobox 2 | EN2 | 14934 |
| enolase 1, (alpha) | ENO1 | 14935 |
| eomesodermin | EOMES | 14936 |
| endothelial PAS domain protein 1 | EPAS1 | 14937 |
| Ets2 repressor factor | ERF | 14938 |
| v-ets avian erythroblastosis virus E26 oncogene | ERG | 14939-14940 |
| homolog | | |
| estrogen receptor 1 | ESR1 | 14941 |
| estrogen receptor 2 (ER beta) | ESR2 | 14942 |
| estrogen related receptor alpha | ESRRA | 14943 |
| estrogen related receptor beta | ESRRB | 14944 |
| estrogen related receptor gamma | ESRRG | 14945 |
| ESX homeobox 1 | ESX1 | 14946 |
| v-ets avian erythroblastosis virus E26 oncogene | ETS1 | 14947 |
| homolog 1 | | |
| v-ets avian erythroblastosis virus E26 oncogene | ETS2 | 14948 |
| homolog 2 | | |
| ets variant 1 | ETV1 | 14949 |
| ets variant 2 | ETV2 | 14950 |
| ets variant 3 | ETV3 | 14951 |
| ets variant 3-like | ETV3L | 14952 |
| ets variant 4 | ETV4 | 14953 |
| ets variant 5 | ETV5 | 14954 |
| ets variant 6 | ETV6 | 14955 |
| ets variant 7 | ETV7 | 14956 |
| even-skipped homeobox 1 | EVX1 | 14957 |
| even-skipped homeobox 2 | EVX2 | 14958 |
| enhancer of zeste 1 poly comb repressive complex 2 | EZH1 | 14959 |
| subunit | | |
| enhancer of zeste 2 poly comb repressive complex 2 | EZH2 | 14960 |
| subunit | | |
| family with sequence similarity 170 member A | FAM170A | 14961 |
| Fer3-like bHLH transcription factor | FERD3L | 14962 |
| FEV (ETS oncogene family) | FEV | 14963 |
| FEZ family zinc finger 1 | FEZF1 | 14964 |
| FEZ family zinc finger 2 | FEZF2 | 14965 |
| folliculogenesis specific bHLH transcription factor | FIGLA | 14966 |
| FLT3-interacting zinc finger 1 | FIZ1 | 14967 |
| Fli-1 proto-oncogene, ETS transcription factor | FLI1 | 14968 |
| FBJ murine osteosarcoma viral oncogene homolog | FOS | 14969 |
| FBJ murine osteosarcoma viral oncogene homolog B | FOSB | 14970 |
| FOS like antigen 1 | FOSL1 | 14971 |
| FOS like antigen 2 | FOSL2 | 14972 |
| forkhead box A1 | FOXA1 | 14973 |
| forkhead box A2 | FOXA2 | 14974 |
| forkhead box A3 | FOXA3 | 14975 |
| forkhead box B1 | FOXB1 | 14976 |
| forkhead box B2 | FOXB2 | 14977 |
| forkhead box C1 | FOXC1 | 14978 |
| forkhead box C2 | FOXC2 | 14979 |
| forkhead box D1 | FOXD1 | 14980 |
| forkhead box D2 | FOXD2 | 14981 |
| forkhead box D3 | FOXD3 | 14982 |
| forkhead box D4 | FOXD4 | 14983 |
| forkhead box D4-like 1 | FOXD4L1 | 14984 |
| forkhead box D4-like 3 | FOXD4L3 | 14985 |
| forkhead box D4-like 4 | FOXD4L4 | 14986 |
| forkhead box D4-like 5 | FOXD4L5 | 14987 |
| forkhead box D4-like 6 | FOXD4L6 | 14988 |
| forkhead box E1 | FOXE1 | 14989 |
| forkhead box E3 | FOXE3 | 14990 |
| forkhead box F1 | FOXF1 | 14991 |
| forkhead box F2 | FOXF2 | 14992 |
| forkhead box G1 | FOXG1 | 14993 |
| forkhead box H1 | FOXH1 | 14994 |
| forkhead box I1 | FOXI1 | 14995 |
| forkhead box I2 | FOXI2 | 14996 |
| forkhead box I3 | FOXI3 | 14997 |
| forkhead box J1 | FOXJ1 | 14998 |
| forkhead box J2 | FOXJ2 | 14999 |
| forkhead box J3 | FOXJ3 | 15000 |
| forkhead box K1 | FOXK1 | 15001 |
| forkhead box K2 | FOXK2 | 15002 |
| forkhead box L1 | FOXL1 | 15003 |
| forkhead box L2 | FOXL2 | 15004 |
| forkhead box M1 | FOXM1 | 15005 |
| forkhead box N1 | FOXN1 | 15006 |
| forkhead box N2 | FOXN2 | 15007 |
| forkhead box N3 | FOXN3 | 15008 |
| forkhead box N4 | FOXN4 | 15009 |
| forkhead box O1 | FOXO1 | 15010 |
| forkhead box O3 | FOXO3 | 15011 |
| forkhead box O4 | FOXO4 | 15012 |
| forkhead box O6 | FOXO6 | 15013 |
| forkhead box P1 | FOXP1 | 15014 |
| forkhead box P2 | FOXP3 | 15015 |
| forkhead box P3 | FOXP4 | 15016 |
| forkhead box P4 | FOXQ1 | 15017 |
| forkhead box Q1 | FOXR1 | 15018 |
| forkhead box R1 | FOXR2 | 15019 |
| forkhead box R2 | FOXS1 | 15020 |
| forkhead box S1 | FOXP3 | 15021 |
| far upstream element binding protein 1 | FUBP1 | 15022 |
| far upstream element (FUSE) binding protein 3 | FUBP3 | 15023 |
| GA binding protein transcription factor alpha subunit | GABPA | 15024 |
| GA binding protein transcription factor, beta subunit 1 | GABPB1 | 15025 |
| GA binding protein transcription factor, beta subunit 2 | GABPB2 | 15026 |
| GATA binding protein 1 (globin transcription factor 1) | GATA1 | 15027 |
| GATA binding protein 2 | GATA2 | 15028 |
| GATA binding protein 3 | GATA3 | 15029 |
| GATA binding protein 4 | GATA4 | 15030 |
| GATA binding protein 5 | GATA5 | 15031 |
| GATA binding protein 6 | GATA6 | 15032 |
| GATA zinc finger domain containing 1 | GATAD1 | 15033 |
| GATA zinc finger domain containing 2 A | GATAD2A | 15034 |
| GATA zinc finger domain containing 2B | GATAD2B | 15035 |
| gastrulation brain homeobox 1 | GBX1 | 15036 |
| gastrulation brain homeobox 2 | GBX2 | 15037 |
| GC-rich sequence DNA-binding factor 2 | GCFC2 | 15038 |
| glial cells missing homolog 1 | GCM1 | 15039 |
| glial cells missing homolog 2 | GCM2 | 15040 |
| growth factor independent 1 transcription repressor | GFI1 | 15041 |
| growth factor independent 1B transcription repressor | GFI1B | 15042 |
| GLI family zinc finger 1 | GLI1 | 15043 |
| GLI family zinc finger 2 | GLI2 | 15044 |
| GLI family zinc finger 3 | GLI3 | 15045 |
| GLI family zinc finger 4 | GLI4 | 15046 |
| GLIS family zinc finger 1 | GLIS1 | 15047 |
| GLIS family zinc finger 2 | GLIS2 | 15048 |
| GLIS family zinc finger 3 | GLIS3 | 15049 |
| glucocorticoid modulatory element binding protein 1 | GMEB1 | 15050 |
| glucocorticoid modulatory element binding protein 2 | GMEB2 | 15051 |
| gon-4-like (C. elegans) | GON4L | 15052 |
| grainyhead like transcription factor 1 | GRHL1 | 15053 |
| grainyhead like transcription factor 2 | GRHL2 | 15054 |
| grainyhead like transcription factor 3 | GRHL3 | 15055 |
| goosecoid homeobox | GSC | 15056 |
| goosecoid homeobox 2 | GSC2 | 15057 |
| GS homeobox 1 | GSX1 | 15058 |
| GS homeobox 2 | GSX2 | 15059 |
| general transcription factor IIi | GTF2I | 15060 |
| general transcription factor IIIA | GTF3A | 15061 |
| GDNF inducible zinc finger protein 1 | GZF1 | 15062 |
| heart and neural crest derivatives expressed 1 | HAND1 | 15063 |
| heart and neural crest derivatives expressed 2 | HAND2 | 15064 |
| HMG-box transcription factor 1 | HBP1 | 15065-15066 |
| highly divergent homeobox | HDX | 15067 |
| helt bHLH transcription factor | HELT | 15068 |
| hes family bHLH transcription factor 1 | HES1 | 15069-15070 |
| hes family bHLH transcription factor 2 | HES2 | 15071 |
| hes family bHLH transcription factor 3 | HES3 | 15072 |
| hes family bHLH transcription factor 4 | HES4 | 15073 |
| hes family bHLH transcription factor 5 | HES5 | 15074 |
| hes family bHLH transcription factor 6 | HES6 | 15075 |
| hes family bHLH transcription factor 7 | HES7 | 15076 |
| HESX homeobox 1 | HESX1 | 15077 |
| hes-related family bHLH transcription factor with | HEY1 | 15078 |
| YRPW motif 1 | | |
| hes-related family bHLH transcription factor with | HEY2 | 15079 |
| YRPW motif 2 | | |
| hes-related family bHLH transcription factor with | HEYL | 15080 |
| YRPW motif-like | | |
| hematopoietically expressed homeobox | HHEX | 15081 |
| hypermethylated in cancer 1 | HIC1 | 15082 |
| hypermethylated in cancer 2 | HIC2 | 15083 |
| hypoxia inducible factor 1, alpha subunit (basic helix- | HIF1A | 15084 |
| loop-helix transcription factor) | | |
| hypoxia inducible factor 3, alpha subunit | HIF3A | 15085 |
| histone H4 transcription factor | HINFP | 15086 |
| human immunodeficiency virus type I enhancer | HIVEP1 | 15087 |
| binding protein 1 | | |
| human immunodeficiency virus type I enhancer | HIVEP2 | 15088 |
| binding protein 2 | | |
| human immunodeficiency virus type I enhancer | HIVEP3 | 15089 |
| binding protein 3 | | |
| HKR1, GLI-Kruppel zinc finger family member | HKR1 | 15090 |
| hepatic leukemia factor | HLF | 15091 |
| helicase-like transcription factor | HLTF | 15092 |
| H2.0-like homeobox | HLX | 15093 |
| homeobox containing 1 | HMBOX1 | 15094 |
| high mobility group 20A | HMG20A | 15095 |
| high mobility group 20B | HMG20B | 15096 |
| high mobility group AT-hook 1 | HMGA1 | 15097 |
| high mobility group AT-hook 2 | HMGA2 | 15098 |
| HMG-box containing 3 | HMGXB3 | 15099 |
| HMG-box containing 4 | HMGXB4 | 15100 |
| H6 family homeobox 1 | HMX1 | 15101 |
| H6 family homeobox 2 | HMX2 | 15102 |
| H6 family homeobox 3 | HMX3 | 15103-15104 |
| HNF1 homeobox A | HNF1A | 15105 |
| HNF1 homeobox B | HNF1B | 15106 |
| hepatocyte nuclear factor 4 alpha | HNF4A | 15107 |
| hepatocyte nuclear factor 4 gamma | HNF4G | 15108 |
| heterogeneous nuclear ribonucleoprotein K | HNRNPK | 15109 |
| homeobox and leucine zipper encoding | HOMEZ | 15110 |
| HOP homeobox | HOPX | 15111 |
| homeobox A1 | HOXA1 | 15112 |
| homeobox A10 | HOXA10 | 15113 |
| homeobox A11 | HOXA11 | 15114 |
| homeobox A13 | HOXA13 | 15115 |
| homeobox A2 | HOXA2 | 15116 |
| homeobox A3 | HOXA3 | 15117 |
| homeobox A4 | HOXA4 | 15118 |
| homeobox A5 | HOXA5 | 15119 |
| homeobox A6 | HOXA6 | 15120 |
| homeobox A7 | HOXA7 | 15121 |
| homeobox A9 | HOXA9 | 15122 |
| homeobox B1 | HOXB1 | 15123 |
| homeobox B13 | HOXB13 | 15124 |
| homeobox B2 | HOXB2 | 15125 |
| homeobox B3 | HOXB3 | 15126 |
| homeobox B4 | HOXB4 | 15127 |
| homeobox B5 | HOXB5 | 15128 |
| homeobox B6 | HOXB6 | 15129 |
| homeobox B7 | HOXB7 | 15130 |
| homeobox B8 | HOXB8 | 15131 |
| homeobox B9 | HOXB9 | 15132 |
| homeobox C10 | HOXC10 | 15133 |
| homeobox C11 | HOXC11 | 15134 |
| homeobox C12 | HOXC12 | 15135 |
| homeobox C13 | HOXC13 | 15136 |
| homeobox C4 | HOXC4 | 15137 |
| homeobox C5 | HOXC5 | 15138 |
| homeobox C6 | HOXC6 | 15139 |
| homeobox C8 | HOXC8 | 15140 |
| homeobox C9 | HOXC9 | 15141 |
| homeobox D1 | HOXD1 | 15142 |
| homeobox D10 | HOXD10 | 15143 |
| homeobox D11 | HOXD11 | 15144 |
| homeobox D12 | HOXD12 | 15145 |
| homeobox D13 | HOXD13 | 15146 |
| homeobox D3 | HOXD3 | 15147 |
| homeobox D4 | HOXD4 | 15148 |
| homeobox D8 | HOXD8 | 15149 |
| homeobox D9 | HOXD9 | 15150 |
| heat shock transcription factor 1 | HSF1 | 15151 |
| heat shock transcription factor 2 | HSF2 | 15152 |
| heat shock transcription factor 4 | HSF4 | 15153 |
| heat shock transcription factor family member 5 | HSF5 | 15154 |
| heat shock transcription factor family, X-linked 1 | HSFX1 | 15155 |
| heat shock transcription factor, Y-linked 1 | HSFY1 | 15156 |
| heat shock transcription factor, Y-linked 2 | HSFY2 | 15156 |
| inhibitor of DNA binding 1, dominant negative helix- | ID1 | 15157 |
| loop-helix protein | | |
| inhibitor of DNA binding 2, dominant negative helix- | ID2 | 15158 |
| loop-helix protein | | |
| inhibitor of DNA binding 3, dominant negative helix- | ID3 | 15159 |
| loop-helix protein | | |
| inhibitor of DNA binding 4, dominant negative helix- | ID4 | 15160 |
| loop-helix protein | | |
| interferon, gamma-inducible protein 16 | IFI16 | 15161 |
| IKAROS family zinc finger 1 | IKZF1 | 15162 |
| IKAROS family zinc finger 2 | IKZF2 | 15163 |
| IKAROS family zinc finger 3 | IKZF3 | 15164 |
| IKAROS family zinc finger 4 | IKZF4 | 15165 |
| IKAROS family zinc finger 5 | IKZF5 | 15166 |
| insulinoma associated 1 | INSM1 | 15167 |
| insulinoma-associated 2 | INSM2 | 15168 |
| interferon regulatory factor 1 | IRF1 | 15169 |
| interferon regulatory factor 2 | IRF2 | 15170 |
| interferon regulatory factor 3 | IRF3 | 15171 |
| interferon regulatory factor 4 | IRF4 | 15172 |
| interferon regulatory factor 5 | IRF5 | 15173 |
| interferon regulatory factor 6 | IRF6 | 15174 |
| interferon regulatory factor 7 | IRF7 | 15175 |
| interferon regulatory factor 8 | IRF8 | 15176 |
| interferon regulatory factor 9 | IRF9 | 15177 |
| iroquois homeobox 1 | IRX1 | 15178 |
| iroquois homeobox 2 | IRX2 | 15179 |
| iroquois homeobox 3 | IRX3 | 15180 |
| iroquois homeobox 4 | IRX4 | 15181 |
| iroquois homeobox 5 | IRX5 | 15182 |
| iroquois homeobox 6 | IRX6 | 15183 |
| ISL LIM homeobox 1 | ISL1 | 15184 |
| ISL LIM homeobox 2 | ISL2 | 15185 |
| intestine specific homeobox | ISX | 15186 |
| jumonji and AT-rich interaction domain containing 2 | JARID2 | 15187 |
| JAZF zinc finger 1 | JAZF1 | 15188 |
| Jun dimerization protein 2 | JDP2 | 15189 |
| jun proto-oncogene | JUN | 15190 |
| jun B proto-oncogene | JUNB | 15191 |
| jun D proto-oncogene | JUND | 15192 |
| K(lysine) acetyltransferase 5 | KAT5 | 15193 |
| lysine acetyltransferase 6A | KAT6A | 15194 |
| lysine acetyltransferase 6B | KAT6B | 15195 |
| lysine acetyltransferase 7 | KAT7 | 15196 |
| lysine acetyltransferase 8 | KAT8 | 15197 |
| potassium channel modulatory factor 1 | KCMF1 | 15198 |
| potassium voltage-gated channel interacting protein 3 | KCNIP3 | 15199 |
| lysine demethylase 2A | KDM2A | 15200 |
| lysine demethylase 5A | KDM5A | 15201 |
| lysine demethylase 5B | KDM5B | 15202 |
| lysine demethylase 5C | KDM5C | 15203 |
| lysine demethylase 5D | KDM5D | 15204 |
| KH-type splicing regulatory protein | KHSRP | 15205 |
| KIAA1549 | KIAA1549 | 15206 |
| Kruppel-like factor 1 (erythroid) | KLF1 | 15207 |
| Kruppel-like factor 10 | KLF10 | 15208 |
| Kruppel-like factor 11 | KLF11 | 15209 |
| Kruppel-like factor 12 | KLF12 | 15210 |
| Kruppel-like factor 13 | KLF13 | 15211 |
| Kruppel-like factor 14 | KLF14 | 15212 |
| Kruppel-like factor 15 | KLF15 | 15213 |
| Kruppel-like factor 16 | KLF16 | 15214 |
| Kruppel-like factor 17 | KLF17 | 15215 |
| Kruppel-like factor 2 | KLF2 | 15216 |
| Kruppel-like factor 3 (basic) | KLF3 | 15217 |
| Kruppel-like factor 4 (gut) | KLF4 | 15218 |
| Kruppel-like factor 5 (intestinal) | KLF5 | 15219 |
| Kruppel-like factor 6 | KLF6 | 15220 |
| Kruppel-like factor 7 (ubiquitous) | KLF7 | 15221 |
| Kruppel-like factor 8 | KLF8 | 15222 |
| Kruppel-like factor 9 | KLF9 | 15223 |
| lysine methyltransferase 2A | KMT2A | 15224 |
| lysine methyltransferase 2B | KMT2B | 15225 |
| lysine methyltransferase 2C | KMT2C | 15226 |
| lysine methyltransferase 2E | KMT2E | 15227 |
| l(3)mbt-like 1 (Drosophila) | L3MBTL1 | 15228 |
| l(3)mbt-like 2 (Drosophila) | L3MBTL2 | 15229 |
| l(3)mbt-like 3 (Drosophila) | L3MBTL3 | 15230 |
| l(3)mbt-like 4 (Drosophila) | L3MBTL4 | 15231 |
| ladybird homeobox 1 | LBX1 | 15232 |
| ladybird homeobox 2 | LBX2 | 15233 |
| ligand dependent nuclear receptor corepressor | LCOR | 15234 |
| ligand dependent nuclear receptor corepressor like | LCORL | 15235 |
| lymphoid enhancer binding factor 1 | LEF1 | 15236 |
| leucine twenty homeobox | LEUTX | 15237 |
| LIM homeobox 1 | LHX1 | 15238 |
| LIM homeobox 2 | LHX2 | 15239 |
| LIM homeobox 3 | LHX3 | 15240 |
| LIM homeobox 4 | LHX4 | 15241 |
| LIM homeobox 5 | LHX5 | 15242 |
| LIM homeobox 6 | LHX6 | 15243 |
| LIM homeobox 8 | LHX8 | 15244 |
| LIM homeobox 9 | LHX9 | 15245 |
| LIM homeobox transcription factor 1, alpha | LMX1A | 15246 |
| LIM homeobox transcription factor 1, beta | LMX1B | 15247 |
| LOC730110 | LOC730110 | |
| leucine rich repeat (in FLII) interacting protein 1 | LRRFIP1 | 15248 |
| leucine rich repeat (in FLII) interacting protein 2 | LRRFIP2 | 15249 |
| Ly 1 antibody reactive | LYAR | 15250 |
| lymphoblastic leukemia associated hematopoiesis | LYL1 | 15251 |
| regulator 1 | | |
| maelstrom spermatogenic transposon silencer | MAEL | 15252 |
| v-maf avian musculoaponeurotic fibrosarcoma | MAF | 15253 |
| oncogene homolog | | |
| MAF1 homolog, negative regulator of RNA | MAF1 | 15254 |
| polymerase III | | |
| v-maf avian musculoaponeurotic fibrosarcoma | MAFA | 15255-15256 |
| oncogene homolog A | | |
| v-maf avian musculoaponeurotic fibrosarcoma | MAFB | 15257 |
| oncogene homolog B | | |
| v-maf avian musculoaponeurotic fibrosarcoma | MAFF | 15258 |
| oncogene homolog F | | |
| v-maf avian musculoaponeurotic fibrosarcoma | MAFG | 15259 |
| oncogene homolog G | | |
| v-maf avian musculoaponeurotic fibrosarcoma | MAFK | 15260 |
| oncogene homolog K | | |
| matrin 3 | MATR3 | 15261 |
| MYC associated factor X | MAX | 15262 |
| MYC associated zinc finger protein | MAZ | 15263 |
| methyl-CpG binding domain protein 1 | MBD1 | 15264 |
| methyl-CpG binding domain protein 2 | MBD2 | 15265 |
| methyl-CpG binding domain protein 3 | MBD3 | 15266 |
| methyl-CpG binding domain protein 3-like 1 | MBD3L1 | 15267 |
| methyl-CpG binding domain protein 3-like 2 | MBD3L2 | 15268 |
| methyl-CpG binding domain 4 DNA glycosylase | MBD4 | 15269 |
| methyl-CpG binding domain protein 5 | MBD5 | 15270 |
| methyl-CpG binding domain protein 6 | MBD6 | 15271 |
| muscleblind like splicing regulator 3 | MBNL3 | 15272 |
| MDS1 and EVI1 complex locus | MECOM | 15273 |
| methyl-CpG binding protein 2 | MECP2 | 15274 |
| myocyte enhancer factor 2A | MEF2A | 15275 |
| myocyte enhancer factor 2B | MEF2B | 15276 |
| myocyte enhancer factor 2C | MEF2C | 15277 |
| myocyte enhancer factor 2D | MEF2D | 15278 |
| Meis homeobox 1 | MEIS1 | 15279 |
| Meis homeobox 2 | MEIS2 | 15280 |
| Meis homeobox 3 | MEIS3 | 15281 |
| Meis homeobox 3 pseudogene 1 | MEIS3P1 | 15282 |
| Meis homeobox 3 pseudogene 2 | MEIS3P2 | 15283 |
| mesenchyme homeobox 1 | MEOX1 | 15284 |
| mesenchyme homeobox 2 | MEOX2 | 15285 |
| mesoderm posterior bHLH transcription factor 1 | MESP1 | 15286 |
| mesoderm posterior bHLH transcription factor 2 | MESP2 | 15287 |
| MGA, MAX dimerization protein | MGA | 15288-15289 |
| MIER1 transcriptional regulator | MIER1 | 15290 |
| MIER family member 2 | MIER2 | 15291 |
| MIER family member 3 | MIER3 | 15292 |
| MIS18 binding protein 1 | MIS18BP1 | 15293 |
| microphthalmia-associated transcription factor | MITF | 15294 |
| Mix paired-like homeobox | MIXL1 | 15295 |
| mohawk homeobox | MKX | 15296 |
| myeloid/lymphoid or mixed-lineage leukemia; | MLLT1 | 15297 |
| translocated to, 1 | | |
| myeloid/lymphoid or mixed-lineage leukemia; | MLLT10 | 15298 |
| translocated to, 10 | | |
| myeloid/lymphoid or mixed-lineage leukemia; | MLLT11 | 15299 |
| translocated to, 11 | | |
| myeloid/lymphoid or mixed-lineage leukemia; | MLLT3 | 15300 |
| translocated to, 3 | | |
| myeloid/lymphoid or mixed-lineage leukemia; | MLLT4 | 15301 |
| translocated to, 4 | | |
| myeloid/lymphoid or mixed-lineage leukemia; | MLLT6 | 15302 |
| translocated to, 6 | | |
| MLX, MAX dimerization protein | MLX | 15303 |
| MLX interacting protein | MLXIP | 15304 |
| MLX interacting protein-like | MLXIPL | 15305 |
| MAX network transcriptional repressor | MNT | 15306 |
| motor neuron and pancreas homeobox 1 | MNX1 | 15307 |
| musculin | MSC | 15308 |
| mesogenin 1 | MSGN1 | 15309 |
| msh homeobox 1 | MSX1 | 15310 |
| msh homeobox 2 | MSX2 | 15311 |
| metastasis associated 1 | MTA1 | 15312 |
| metastasis associated 1 family member 2 | MTA2 | 15313 |
| metastasis associated 1 family member 3 | MTA3 | 15314 |
| metal-regulatory transcription factor 1 | MTF1 | 15315 |
| metal response element binding transcription factor 2 | MTF2 | 15316 |
| MAX dimerization protein 1 | MXD1 | 15317 |
| MAX dimerization protein 3 | MXD3 | 15318 |
| MAX dimerization protein 4 | MXD4 | 15319 |
| MAX interactor 1, dimerization protein | MXI1 | 15320 |
| v-myb avian myeloblastosis viral oncogene homolog | MYB | 15321 |
| v-myb avian myeloblastosis viral oncogene homolog- | MYBL1 | 15322 |
| like 1 | | |
| v-myb avian myeloblastosis viral oncogene homolog- | MYBL2 | 15323 |
| like 2 | | |
| v-myc avian myelocytomatosis viral oncogene | MYC | 15324 |
| homolog | | |
| v-myc avian myelocytomatosis viral oncogene lung | MYCL | 15325 |
| carcinoma derived homolog | | |
| MYCL pseudogene 1 | MYCLP1 | 15326 |
| v-myc avian myelocytomatosis viral oncogene | MYCN | 15327 |
| neuroblastoma derived homolog | | |
| myogenic factor 5 | MYF5 | 15328 |
| myogenic factor 6 | MYF6 | 15329 |
| myoneurin | MYNN | 15330 |
| myogenic differentiation 1 | MYOD1 | 15331 |
| myogenin (myogenic factor 4) | MYOG | 15332 |
| myelin regulatory factor | MYRF | 15333 |
| Myb-like, SWIRM and MPN domains 1 | MYSM1 | 15334 |
| myelin transcription factor 1 | MYT1 | 15335-15336 |
| myelin transcription factor 1 like | MYT1L | 15337 |
| myeloid zinc finger 1 | MZF1 | 15338 |
| Nanog homeobox | NANOG | 15339 |
| NANOG neighbor homeobox | NANOGNB | 15340 |
| Nanog homeobox pseudogene 1 | NANOGP1 | 15341 |
| Nanog homeobox pseudogene 8 | NANOGP8 | 15342 |
| nuclear receptor coactivator 1 | NCOA1 | 15343 |
| nuclear receptor coactivator 2 | NCOA2 | 15344 |
| nuclear receptor coactivator 3 | NCOA3 | 15345 |
| nuclear receptor coactivator 4 | NCOA4 | 15346 |
| nuclear receptor coactivator 5 | NCOA5 | 15347 |
| nuclear receptor coactivator 6 | NCOA6 | 15348 |
| nuclear receptor coactivator 7 | NCOA7 | 15349 |
| nuclear receptor corepressor 1 | NCOR1 | 15350 |
| nuclear receptor corepressor 2 | NCOR2 | 15351 |
| neuronal differentiation 1 | NEUROD1 | 15352 |
| neuronal differentiation 2 | NEUROD2 | 15353 |
| neuronal differentiation 4 | NEUROD4 | 15354 |
| neuronal differentiation 6 | NEUROD6 | 15355 |
| neuro genin 1 | NEUROG1 | 15356 |
| neuro genin 2 | NEUROG2 | 15357 |
| neuro genin 3 | NEUROG3 | 15358 |
| nuclear factor of activated T-cells 5, tonicity- | NFAT5 | 15359 |
| responsive | | |
| nuclear factor of activated T-cells, cytoplasmic, | NFATC1 | 15360 |
| calcineurin-dependent 1 | | |
| nuclear factor of activated T-cells, cytoplasmic, | NFATC2 | 15361 |
| calcineurin-dependent 2 | | |
| nuclear factor of activated T-cells, cytoplasmic, | NFATC3 | 15362 |
| calcineurin-dependent 3 | | |
| nuclear factor of activated T-cells, cytoplasmic, | NFATC4 | 15363 |
| calcineurin-dependent 4 | | |
| nuclear factor, erythroid 2 | NFE2 | 15364 |
| nuclear factor, erythroid 2 like 1 | NFE2L1 | 15365 |
| nuclear factor, erythroid 2 like 2 | NFE2L2 | 15366 |
| nuclear factor, erythroid 2 like 3 | NFE2L3 | 15367 |
| nuclear factor I/A | NFIA | 15368 |
| nuclear factor I/B | NFIB | 15369 |
| nuclear factor I/C (CCAAT-binding transcription | NFIC | 15370 |
| factor) | | |
| nuclear factor, interleukin 3 regulated | NFIL3 | 15371 |
| nuclear factor I/X (CCAAT-binding transcription | NFIX | 15372 |
| factor) | | |
| nuclear factor of kappa light polypeptide gene | NFKB1 | 15373 |
| enhancer in B-cells 1 | | |
| nuclear factor of kappa light polypeptide gene | NFKB2 | 15374 |
| enhancer in B-cells 2 (p49/p100) | | |
| nuclear factor of kappa light polypeptide gene | NFKBIA | 15375 |
| enhancer in B-cells inhibitor, alpha | | |
| nuclear factor of kappa light polypeptide gene | NFKBIB | 15376 |
| enhancer in B-cells inhibitor, beta | | |
| nuclear factor of kappa light polypeptide gene | NFKBID | 15377 |
| enhancer in B-cells inhibitor, delta | | |
| nuclear factor of kappa light polypeptide gene | NFKBIE | 15378 |
| enhancer in B-cells inhibitor, epsilon | | |
| nuclear factor of kappa light polypeptide gene | NFKBIL1 | 15379 |
| enhancer in B-cells inhibitor-like 1 | | |
| nuclear factor of kappa light polypeptide gene | NFKBIZ | 15380 |
| enhancer in B-cells inhibitor, zeta | | |
| nuclear factor related to kappaB binding protein | NFRKB | 15381 |
| nuclear transcription factor, X-box binding 1 | NFX1 | 15382 |
| nuclear transcription factor, X-box binding-like 1 | NFXL1 | 15383 |
| nuclear transcription factor Y subunit alpha | NFYA | 15384 |
| nuclear transcription factor Y subunit beta | NFYB | 15385 |
| nuclear transcription factor Y subunit gamma | NFYC | 15386 |
| nescient helix-loop-helix 1 | NHLH1 | 15387 |
| nescient helix-loop-helix 2 | NHLH2 | 15388 |
| NFKB repressing factor | NKRF | 15389 |
| NK1 homeobox 1 | NKX1-1 | 15390 |
| NK1 homeobox 2 | NKX1-2 | 15391 |
| NK2 homeobox 1 | NKX2-1 | 15392 |
| NK2 homeobox 2 | NKX2-2 | 15393 |
| NK2 homeobox 3 | NKX2-3 | 15394 |
| NK2 homeobox 4 | NKX2-4 | 15395 |
| NK2 homeobox 5 | NKX2-5 | 15396 |
| NK2 homeobox 6 | NKX2-6 | 15397 |
| NK2 homeobox 8 | NKX2-8 | 15398 |
| NK3 homeobox 1 | NKX3-1 | 15399 |
| NK3 homeobox 2 | NKX3-2 | 15400 |
| NK6 homeobox 1 | NKX6-1 | 15401 |
| NK6 homeobox 2 | NKX6-2 | 15402 |
| NK6 homeobox 3 | NKX6-3 | 15403 |
| NOBOX oogenesis homeobox | NOBOX | 15404 |
| NOC3 like DNA replication regulator | NOC3L | 15405 |
| nucleolar complex associated 4 homolog | NOC4L | 15406 |
| non-POU domain containing, octamer-binding | NONO | 15407 |
| notochord homeobox | NOTO | 15408 |
| neuronal PAS domain protein 1 | NPAS1 | 15409 |
| neuronal PAS domain protein 2 | NPAS2 | 15410 |
| neuronal PAS domain protein 3 | NPAS3 | 15411 |
| neuronal PAS domain protein 4 | NPAS4 | 15412 |
| nuclear receptor subfamily 0 group B member 1 | NR0B1 | 15413 |
| nuclear receptor subfamily 0 group B member 2 | NR0B2 | 15414 |
| nuclear receptor subfamily 1 group D member 1 | NR1D1 | 15415 |
| nuclear receptor subfamily 1 group D member 2 | NR1D2 | 15416 |
| nuclear receptor subfamily 1 group H member 2 | NR1H2 | 15417 |
| nuclear receptor subfamily 1 group H member 3 | NR1H3 | 15418 |
| nuclear receptor subfamily 1 group H member 4 | NR1H4 | 15419 |
| nuclear receptor subfamily 1 group I member 2 | NR1I2 | 15420 |
| nuclear receptor subfamily 1 group I member 3 | NR1I3 | 15421 |
| nuclear receptor subfamily 2 group C member 1 | NR2C1 | 15422 |
| nuclear receptor subfamily 2 group C member 2 | NR2C2 | 15423 |
| nuclear receptor subfamily 2 group E member 1 | NR2E1 | 15424 |
| nuclear receptor subfamily 2 group E member 3 | NR2E3 | 15425 |
| nuclear receptor subfamily 2 group F member 1 | NR2F1 | 15426 |
| nuclear receptor subfamily 2 group F member 2 | NR2F2 | 15427 |
| nuclear receptor subfamily 2 group F member 6 | NR2F6 | 15428 |
| nuclear receptor subfamily 3 group C member 1 | NR3C1 | 15429 |
| nuclear receptor subfamily 3 group C member 2 | NR3C2 | 15430 |
| nuclear receptor subfamily 4 group A member 1 | NR4A1 | 15431 |
| nuclear receptor subfamily 4 group A member 2 | NR4A2 | 15432 |
| nuclear receptor subfamily 4 group A member 3 | NR4A3 | 15433 |
| nuclear receptor subfamily 5 group A member 1 | NR5A1 | 15434 |
| nuclear receptor subfamily 5 group A member 2 | NR5A2 | 15435 |
| nuclear receptor subfamily 6 group A member 1 | NR6A1 | 15436 |
| nuclear respiratory factor 1 | NRF1 | 15437-15438 |
| neural retina leucine zipper | NRL | 15439 |
| oligodendrocyte transcription factor 1 | OLIG1 | 15440 |
| oligodendrocyte lineage transcription factor 2 | OLIG2 | 15441 |
| oligodendrocyte transcription factor 3 | OLIG3 | 15442 |
| one cut homeobox 1 | ONECUT1 | 15443 |
| one cut homeobox 2 | ONECUT2 | 15444 |
| one cut homeobox 3 | ONECUT3 | 15445 |
| odd-skipped related transciption factor 1 | OSR1 | 15446 |
| odd-skipped related transciption factor 2 | OSR2 | 15447 |
| orthopedia homeobox | OTP | 15448 |
| orthodenticle homeobox 1 | OTX1 | 15449 |
| orthodenticle homeobox 2 | OTX2 | 15450 |
| ovo like zinc finger 1 | OVOL1 | 15451 |
| ovo like zinc finger 2 | OVOL2 | 15452 |
| ovo like zinc finger 3 | OVOL3 | 15453 |
| poly(ADP-ribose) polymerase 1 | PARP1 | 15454 |
| poly(ADP-ribose) polymerase family member 12 | PARP12 | 15455 |
| POZ/BTB and AT hook containing zinc finger 1 | PATZ1 | 15456 |
| PRKC, apoptosis, WT1, regulator | PAWR | 15457 |
| paired box 1 | PAX1 | 15458 |
| paired box 2 | PAX2 | 15459 |
| paired box 3 | PAX3 | 15460 |
| paired box 4 | PAX4 | 15461 |
| paired box 5 | PAX5 | 15462 |
| paired box 6 | PAX6 | 15463 |
| paired box 7 | PAX7 | 15464 |
| paired box 8 | PAX8 | 15465 |
| paired box 9 | PAX9 | 15466 |
| PAX3 and PAX7 binding protein 1 | PAXBP1 | 15467 |
| polybromo 1 | PBRM1 | 15468 |
| pre-B-cell leukemia homeobox 1 | PBX1 | 15469 |
| pre-B-cell leukemia homeobox 2 | PBX2 | 15470 |
| pre-B-cell leukemia homeobox 3 | PBX3 | 15471 |
| pre-B-cell leukemia homeobox 4 | PBX4 | 15472 |
| poly(rC) binding protein 1 | PCBP1 | 15473 |
| poly(rC) binding protein 2 | PCBP2 | 15474 |
| poly(rC) binding protein 3 | PCBP3 | 15475 |
| poly(rC) binding protein 4 | PCBP4 | 15476 |
| poly comb group ring finger 6 | PCGF6 | 15477 |
| pancreatic and duodenal homeobox 1 | PDX1 | 15478-15479 |
| paternally expressed 3 | PEG3 | 15480 |
| progesterone receptor | PGR | 15481 |
| prohibitin | PHB | 15482 |
| prohibitin 2 | PHB2 | 15483 |
| PHD finger protein 20 | PHF20 | 15484 |
| PHD finger protein 5A | PHF5A | 15485 |
| paired like homeobox 2a | PHOX2A | 15486 |
| paired like homeobox 2b | PHOX2B | 15487 |
| putative homeodomain transcription factor 1 | PHTF1 | 15488 |
| putative homeodomain transcription factor 2 | PHTF2 | 15489 |
| paired like homeodomain 1 | PITX1 | 15490 |
| paired like homeodomain 2 | PITX2 | 15491 |
| paired like homeodomain 3 | PITX3 | 15492 |
| PBX/knotted 1 homeobox 1 | PKNOX1 | 15493 |
| PBX/knotted 1 homeobox 2 | PKNOX2 | 15494 |
| PLAG1 zinc finger | PLAG1 | 15495 |
| PLAG1 like zinc finger 1 | PLAGL1 | 15496 |
| PLAG1 like zinc finger 2 | PLAGL2 | 15497 |
| pleckstrin | PLEK | 15498 |
| promyelocytic leukaemia zinc finger | PLZF | 15499 |
| pogo transposable element with ZNF domain | POGZ | 15500 |
| POU class 1 homeobox 1 | POU1F1 | 15501 |
| POU class 2 associating factor 1 | POU2AF1 | 15502 |
| POU class 2 homeobox 1 | POU2F1 | 15503 |
| POU class 2 homeobox 2 | POU2F2 | 15504 |
| POU class 2 homeobox 3 | POU2F3 | 15505 |
| POU class 3 homeobox 1 | POU3F1 | 15506 |
| POU class 3 homeobox 2 | POU3F2 | 15507 |
| POU class 3 homeobox 3 | POU3F3 | 15508 |
| POU class 3 homeobox 4 | POU3F4 | 15509 |
| POU class 4 homeobox 1 | POU4F1 | 15510 |
| POU class 4 homeobox 2 | POU4F2 | 15511 |
| POU class 4 homeobox 3 | POU4F3 | 15512 |
| POU class 5 homeobox 1 | POU5F1 | 15513 |
| POU class 5 homeobox 1B | POU5F1B | 15514 |
| POU domain class 5, transcription factor 2 | POU5F2 | 15515 |
| POU class 6 homeobox 1 | POU6F1 | 15516 |
| POU class 6 homeobox 2 | POU6F2 | 15517 |
| peroxisome proliferator activated receptor alpha | PPARA | 15518 |
| peroxisome proliferator activated receptor delta | PPARD | 15519 |
| peroxisome proliferator activated receptor gamma | PPARG | 15520 |
| protein phosphatase 1 regulatory subunit 13 like | PPP1R13L | 15521 |
| PR domain 1 | PRDM1 | 15522 |
| PR domain 10 | PRDM10 | 15523 |
| PR domain 11 | PRDM11 | 15524 |
| PR domain 12 | PRDM12 | 15525 |
| PR domain 13 | PRDM13 | 15526 |
| PR domain 14 | PRDM14 | 15527 |
| PR domain 15 | PRDM15 | 15528 |
| PR domain 16 | PRDM16 | 15529 |
| PR domain 2 | PRDM2 | 15530 |
| PR domain 4 | PRDM4 | 15531 |
| PR domain 5 | PRDM5 | 15532 |
| PR domain 6 | PRDM6 | 15533 |
| PR domain 7 | PRDM7 | 15534 |
| PR domain 8 | PRDM8 | 15535 |
| PR domain 9 | PRDM9 | 15536 |
| prolactin regulatory element binding | PREB | 15537 |
| PROP paired-like homeobox 1 | PROP1 | 15538 |
| prospero homeobox 1 | PROX1 | 15539 |
| prospero homeobox 2 | PROX2 | 15540 |
| paired related homeobox 1 | PRRX1 | 15541 |
| paired related homeobox 2 | PRRX2 | 15542 |
| paraspeckle component 1 | PSPC1 | 15543 |
| pancreas specific transcription factor, 1a | PTF1A | 15544 |
| purine-rich element binding protein A | PURA | 15545 |
| purine-rich element binding protein B | PURB | 15546 |
| purine-rich element binding protein G | PURG | 15547 |
| retinoic acid receptor alpha | RARA | 15548 |
| retinoic acid receptor beta | RARB | 15549 |
| retinoic acid receptor gamma | RARG | 15550 |
| retina and anterior neural fold homeobox | RAX | 15551-15552 |
| retina and anterior neural fold homeobox 2 | RAX2 | 15553 |
| RB associated KRAB zinc finger | RBAK | 15554 |
| RNA binding motif protein 22 | RBM22 | 15555 |
| recombination signal binding protein for | RBPJ | 15556 |
| immunoglobulin kappa J region | | |
| recombination signal binding protein for | RBPJL | 15557 |
| immunoglobulin kappa J region-like | | |
| ring finger and CCCH-type domains 1 | RC3H1 | 15558 |
| ring finger and CCCH-type domains 2 | RC3H2 | 15559 |
| REST corepressor 1 | RCOR1 | 15560 |
| REST corepressor 2 | RCOR2 | 15561 |
| REST corepressor 3 | RCOR3 | 15562 |
| v-rel avian reticuloendothcliosis viral oncogene | REL | 15563 |
| homolog | | |
| v-rel avian reticuloendothcliosis viral oncogene | RELA | 15564 |
| homolog A | | |
| v-rel avian reticuloendothcliosis viral oncogene | RELB | 15565 |
| homolog B | | |
| arginine-glutamic acid di peptide (RE) repeats | RERE | 15566 |
| RE1-silencing transcription factor | REST | 15567 |
| regulatory factor X1 | RFX1 | 15568 |
| regulatory factor X2 | RFX2 | 15569 |
| regulatory factor X3 | RFX3 | 15570 |
| regulatory factor X4 | RFX4 | 15571 |
| regulatory factor X5 | RFX5 | 15572 |
| regulatory factor X6 | RFX6 | 15573 |
| regulatory factor X7 | RFX7 | 15574 |
| RFX family member 8, lacking RFX DNA binding | RFX8 | 15575 |
| domain | | |
| regulatory factor X associated ankyrin containing | RFXANK | 15576 |
| protein | | |
| regulatory factor X associated protein | RFXAP | 15577 |
| Rhox homeobox family member 1 | RHOXF1 | 15578 |
| Rhox homeobox family member 2 | RHOXF2 | 15579 |
| Rhox homeobox family member 2B | RHOXF2B | 15580 |
| rearranged L-myc fusion | RLF | 15581-15582 |
| RAR related orphan receptor A | RORA | 15583 |
| RAR related orphan receptor B | RORB | 15584 |
| RAR related orphan receptor C | RORC | 15585 |
| retinoic acid receptor-related orphan nuclear receptor | RORgT | 15586 |
| gamma | | |
| ras responsive element binding protein 1 | RREB1 | 15587 |
| runt related transcription factor 1 | RUNX1 | 15588 |
| runt related transcription factor 1; translocated to, 1 | RUNX1T1 | 15589 |
| (cyclin D related) | | |
| runt related transcription factor 2 | RUNX2 | 15590 |
| runt related transcription factor 3 | RUNX3 | 15591 |
| retinoid X receptor alpha | RXRA | 15592 |
| retinoid X receptor beta | RXRB | 15593 |
| retinoid X receptor gamma | RXRG | 15594 |
| spalt-like transcription factor 1 | SALL1 | 15595 |
| spalt-like transcription factor 2 | SALL2 | 15596 |
| spalt-like transcription factor 3 | SALL3 | 15597 |
| spalt-like transcription factor 4 | SALL4 | 15598 |
| SATB homeobox 1 | SATB1 | 15599 |
| SATB homeobox 2 | SATB2 | 15600 |
| S-phase cyclin A-associated protein in the ER | SCAPER | 15601 |
| scratch family zinc finger 1 | SCRT1 | 15602 |
| scratch family zinc finger 2 | SCRT2 | 15603 |
| scleraxis bHLH transcription factor | SCX | 15604 |
| SEBOX homeobox | SEBOX | 15605 |
| SET binding protein 1 | SETBP1 | 15606 |
| splicing factor proline/glutamine-rich | SFPQ | 15607 |
| short stature homeobox | SHOX | 15608 |
| short stature homeobox 2 | SHOX2 | 15609 |
| single-minded family bHLH transcription factor 1 | SIM1 | 15610 |
| single-minded family bHLH transcription factor 2 | SIM2 | 15611 |
| SIX homeobox 1 | SIX1 | 15612 |
| SIX homeobox 2 | SIX2 | 15613 |
| SIX homeobox 3 | SIX3 | 15614 |
| SIX homeobox 4 | SIX4 | 15615 |
| SIX homeobox 5 | SIX5 | 15616 |
| SIX homeobox 6 | SIX6 | 15617 |
| SKI proto-oncogene | SKI | 15618 |
| SKI-like proto-oncogene | SKIL | 15619 |
| SKI family transcriptional corepressor 1 | SKOR1 | 15620 |
| SKI family transcriptional corepressor 2 | SKOR2 | 15621 |
| solute carrier family 30 (zinc transporter), member 9 | SLC30A9 | 15622 |
| SMAD family member 1 | SMAD1 | 15623 |
| SMAD family member 2 | SMAD2 | 15624 |
| SMAD family member 3 | SMAD3 | 15625 |
| SMAD family member 4 | SMAD4 | 15626 |
| SMAD family member 5 | SMAD5 | 15627 |
| SMAD family member 6 | SMAD6 | 15628 |
| SMAD family member 7 | SMAD7 | 15629 |
| SMAD family member 9 | SMAD9 | 15630 |
| SWI/SNF related, matrix associated, actin dependent | SMARCA1 | 15631 |
| regulator of chromatin, subfamily a, member 1 | | |
| SWI/SNF related, matrix associated, actin dependent | SMARCA2 | 15632 |
| regulator of chromatin, subfamily a, member 2 | | |
| SWI/SNF related, matrix associated, actin dependent | SMARCA4 | 15633 |
| regulator of chromatin, subfamily a, member 4 | | |
| SWI/SNF related, matrix associated, actin dependent | SMARCA5 | 15634 |
| regulator of chromatin, subfamily a, member 5 | | |
| SWI/SNF-related, matrix-associated actin-dependent | SMARCAD1 | 15635 |
| regulator of chromatin, subfamily a, containing | | |
| DEAD/H box 1 | | |
| SWI/SNF related, matrix associated, actin dependent | SMARCAL1 | 15636 |
| regulator of chromatin, subfamily a-like 1 | | |
| SWI/SNF related, matrix associated, actin dependent | SMARCB1 | 15637 |
| regulator of chromatin, subfamily b, member 1 | | |
| SWI/SNF related, matrix associated, actin dependent | SMARCC1 | 15638 |
| regulator of chromatin, subfamily c, member 1 | | |
| SWI/SNF related, matrix associated, actin dependent | SMARCC2 | 15639 |
| regulator of chromatin, subfamily c, member 2 | | |
| SWI/SNF related, matrix associated, actin dependent | SMARCD1 | 15640 |
| regulator of chromatin, subfamily d, member 1 | | |
| SWI/SNF related, matrix associated, actin dependent | SMARCD2 | 15641 |
| regulator of chromatin, subfamily d, member 2 | | |
| SWI/SNF related, matrix associated, actin dependent | SMARCD3 | 15642 |
| regulator of chromatin, subfamily d, member 3 | | |
| SWI/SNF related, matrix associated, actin dependent | SMARCE1 | 15643 |
| regulator of chromatin, subfamily e, member 1 | | |
| snail family zinc finger 1 | SNAI1 | 15644 |
| snail family zinc finger 2 | SNAI2 | 15645 |
| snail family zinc finger 3 | SNAI3 | 15646 |
| small nuclear RNA activating complex polypeptide 4 | SNAPC4 | 15647 |
| spermatogenesis and oogenesis specific basic helix- | SOHLH1 | 15648 |
| loop-helix 1 | | |
| spermatogenesis and oogenesis specific basic helix- | SOHLH2 | 15649 |
| loop-helix 2 | | |
| SRY-box 1 | SOX1 | 15650 |
| SRY-box 10 | SOX10 | 15651 |
| SRY-box 11 | SOX11 | 15652 |
| SRY-box 12 | SOX12 | 15653 |
| SRY-box 13 | SOX13 | 15654 |
| SRY-box 14 | SOX14 | 15655 |
| SRY-box 15 | SOX15 | 15656 |
| SRY-box 17 | SOX17 | 15657 |
| SRY-box 18 | SOX18 | 15658 |
| SRY-box 2 | SOX2 | 15659 |
| SRY-box 21 | SOX21 | 15660 |
| SRY-box 3 | SOX3 | 15661 |
| SRY-box 30 | SOX30 | 15662 |
| SRY-box 4 | SOX4 | 15663 |
| SRY-box 5 | SOX5 | 15664 |
| SRY-box 6 | SOX6 | 15665 |
| SRY-box 7 | SOX7 | 15666 |
| SRY-box 8 | SOX8 | 15667 |
| SRY-box 9 | SOX9 | 15668 |
| Sp1 transcription factor | SP1 | 15669-15670 |
| SP100 nuclear antigen | SP100 | 15671 |
| SP110 nuclear body protein | SP110 | 15672 |
| SP140 nuclear body protein | SP140 | 15673 |
| SP140 nuclear body protein like | SP140L | 15674 |
| Sp2 transcription factor | SP2 | 15675 |
| Sp3 transcription factor | SP3 | 15676 |
| Sp4 transcription factor | SP4 | 15677 |
| Sp5 transcription factor | SP5 | 15678 |
| Sp6 transcription factor | SP6 | 15679 |
| Sp7 transcription factor | SP7 | 15680 |
| Sp8 transcription factor | SP8 | 15681 |
| Sp9 transcription factor | SP9 | 15682 |
| SAM pointed domain containing ETS transcription | SPDEF | 15683 |
| factor | | |
| Spi-1 proto-oncogene | SPI1 | 15684 |
| Spi-B transcription factor (Spi-1/PU.1 related) | SPIB | 15685 |
| Spi-C transcription factor (Spi-1/PU.1 related) | SPIC | 15686 |
| spermatogenic leucine zipper 1 | SPZ1 | 15687 |
| sterol regulatory element binding transcription factor 1 | SREBF1 | 15688 |
| sterol regulatory element binding transcription factor 2 | SREBF2 | 15689 |
| serum response factor | SRF | 15690 |
| sex determining region Y | SRY | 15691 |
| structure specific recognition protein 1 | SSRP1 | 15692 |
| suppression of tumorigenicity 18, zinc finger | ST18 | 15693 |
| signal transducer and activator of transcription 1 | STAT1 | 15694 |
| signal transducer and activator of transcription 2 | STAT2 | 15695 |
| signal transducer and activator of transcription 3 | STAT3 | 15696 |
| (acute-phase response factor) | | |
| signal transducer and activator of transcription 4 | STAT4 | 15697 |
| signal transducer and activator of transcription 5 | STAT5 | 15698 |
| signal transducer and activator of transcription 5A | STAT5A | 15699 |
| signal transducer and activator of transcription 5B | STAT5B | 15700 |
| signal transducer and activator of transcription 6, | STAT6 | 15701 |
| interleukin-4 induced | | |
| transcriptional adaptor 2A | TADA2A | 15702 |
| transcriptional adaptor 2B | TADA2B | 15703 |
| TATA-box binding protein associated factor 1 | TAF1 | 15704 |
| T-cell acute lymphocytic leukemia 1 | TAL1 | 15705 |
| T-cell acute lymphocytic leukemia 2 | TAL2 | 15706 |
| Taxi (human T-cell leukemia virus type I) binding | TAX1BP1 | 15707 |
| protein 1 | | |
| Taxi (human T-cell leukemia virus type I) binding | TAX1BP3 | 15708 |
| protein 3 | | |
| T-box transcription factor T-bet | Tbet | 15709 |
| TATA-box binding protein | TBP | 15710 |
| TATA-box binding protein like 1 | TBPL1 | 15711 |
| TATA-box binding protein like 2 | TBPL2 | 15712 |
| T-box, brain 1 | TBR1 | 15713 |
| T-box 1 | TBX1 | 15714 |
| T-box 10 | TBX10 | 15715 |
| T-box 15 | TBX15 | 15716 |
| T-box 18 | TBX18 | 15717 |
| T-box 19 | TBX19 | 15718 |
| T-box 2 | TBX2 | 15719 |
| T-box 20 | TBX20 | 15720 |
| T-box 21 | TBX21 | 15721 |
| T-box 22 | TBX22 | 15722 |
| T-box 3 | TBX3 | 15723 |
| T-box 4 | TBX4 | 15724 |
| T-box 5 | TBX5 | 15725 |
| T-box 6 | TBX6 | 15726 |
| transcription factor 12 | TCF12 | 15727 |
| transcription factor 15 (basic helix-loop-helix) | TCF15 | 15728 |
| transcription factor 19 | TCF19 | 15729 |
| transcription factor 20 (AR1) | TCF20 | 15730 |
| transcription factor 21 | TCF21 | 15731 |
| transcription factor 23 | TCF23 | 15732 |
| transcription factor 24 | TCF24 | 15733 |
| transcription factor 25 (basic helix-loop-helix) | TCF25 | 15734 |
| transcription factor 3 | TCF3 | 15735 |
| transcription factor 4 | TCF4 | 15736 |
| transcription factor 7 (T-cell specific, HMG-box, | TCF7 | 15737 |
| TCF1) | | |
| transcription factor 7 like 1 | TCF7L1 | 15738 |
| transcription factor 7 like 2 | TCF7L2 | 15739 |
| transcription factor-like 5 (basic helix-loop-helix) | TCFL5 | 15740 |
| TEA domain transcription factor 1 | TEAD1 | 15741 |
| TEA domain transcription factor 2 | TEAD2 | 15742 |
| TEA domain transcription factor 3 | TEAD3 | 15743 |
| TEA domain transcription factor 4 | TEAD4 | 15744 |
| thyrotrophic embryonic factor | TEF | 15745 |
| telomeric repeat binding factor (NIMA-interacting) 1 | TERF1 | 15746 |
| telomeric repeat binding factor 2 | TERF2 | 15747 |
| tet methylcytosine dioxygenase 1 | TET1 | 15748 |
| tet methylcytosine dioxygenase 2 | TET2 | 15749 |
| tet methylcytosine dioxygenase 3 | TET3 | 15750 |
| transcription factor A, mitochondrial | TFAM | 15751 |
| transcription factor AP-2 alpha (activating enhancer | TFAP2A | 15752 |
| binding protein 2 alpha) | | |
| transcription factor AP-2 beta (activating enhancer | TFAP2B | 15753 |
| binding protein 2 beta) | | |
| transcription factor AP-2 gamma (activating enhancer | TFAP2C | 15754 |
| binding protein 2 gamma) | | |
| transcription factor AP-2 delta (activating enhancer | TFAP2D | 15755 |
| binding protein 2 delta) | | |
| transcription factor AP-2 epsilon (activating enhancer | TFAP2E | 15756 |
| binding protein 2 epsilon) | | |
| transcription factor AP-4 (activating enhancer binding | TFAP4 | 15757 |
| protein 4) | | |
| transcription factor B1, mitochondrial | TFB1M | 15758 |
| transcription factor B2, mitochondrial | TFB2M | 15759 |
| transcription factor CP2 | TFCP2 | 15760 |
| transcription factor CP2-like 1 | TFCP2L1 | 15761 |
| transcription factor Dp-1 | TFDP1 | 15762 |
| transcription factor Dp-2 (E2F dimerization partner 2) | TFDP2 | 15763 |
| transcription factor Dp family member 3 | TFDP3 | 15764 |
| transcription factor binding to IGHM enhancer 3 | TFE3 | 15765 |
| transcription factor EB | TFEB | 15766 |
| transcription factor EC | TFEC | 15767 |
| TGFB induced factor homeobox 1 | TGIF1 | 15768 |
| TGFB induced factor homeobox 2 | TGIF2 | 15769 |
| TGFB induced factor homeobox 2 like, X-linked | TGIF2LX | 15770 |
| TGFB induced factor homeobox 2 like, Y-linked | TGIF2LY | 15771 |
| THAP domain containing, apoptosis associated protein | THAP1 | 15772 |
| 1 | | |
| THAP domain containing 10 | THAP10 | 15773 |
| THAP domain containing 11 | THAP11 | 15774 |
| THAP domain containing 12 | THAP12 | 15775 |
| THAP domain containing, apoptosis associated protein | THAP2 | 15776 |
| 2 | | |
| THAP domain containing, apoptosis associated protein | THAP3 | 15777 |
| 3 | | |
| THAP domain containing 4 | THAP4 | 15778 |
| THAP domain containing 5 | THAP5 | 15779 |
| THAP domain containing 6 | THAP6 | 15780 |
| THAP domain containing 7 | THAP7 | 15781 |
| THAP domain containing 8 | THAP8 | 15782 |
| THAP domain containing 9 | THAP9 | 15783 |
| Th inducing POZ-Kruppel Factor | ThPOK | 15784 |
| thyroid hormone receptor, alpha | THRA | 15785 |
| thyroid hormone receptor, beta | THRB | 15786 |
| T-cell leukemia homeobox 1 | TLX1 | 15787 |
| T-cell leukemia homeobox 2 | TLX2 | 15788 |
| T-cell leukemia homeobox 3 | TLX3 | 15789 |
| target of EGR1, member 1 (nuclear) | TOE1 | 15790 |
| tonsoku-like, DNA repair protein | TONSL | 15791 |
| topoisomerase I binding, arginine/serine-rich, E3 | TOPORS | 15792 |
| ubiquitin protein ligase | | |
| thymocyte selection associated high mobility group | TOX | 15793 |
| box | | |
| TOX high mobility group box family member 2 | TOX2 | 15794 |
| TOX high mobility group box family member 3 | TOX3 | 15795 |
| TOX high mobility group box family member 4 | TOX4 | 15796 |
| tumor protein p53 | TP53 | 15797 |
| tumor protein p63 | TP63 | 15798 |
| tumor protein p73 | TP73 | 15799 |
| tetra-peptide repeat homeobox 1 | TPRX1 | 15800 |
| tetra-peptide repeat homeobox-like | TPRXL | 15801 |
| transcriptional regulating factor 1 | TRERF1 | 15802 |
| trichorhinophalangeal syndrome I | TRPS1 | 15803 |
| TSC22 domain family member 1 | TSC22D1 | 15804 |
| TSC22 domain family member 2 | TSC22D2 | 15805 |
| TSC22 domain family member 3 | TSC22D3 | 15806 |
| TSC22 domain family member 4 | TSC22D4 | 15807 |
| teashirt zinc finger homeobox 1 | TSHZ1 | 15808 |
| teashirt zinc finger homeobox 2 | TSHZ2 | 15809 |
| teashirt zinc finger homeobox 3 | TSHZ3 | 15810 |
| transcription termination factor, RNA polymerase I | TTF1 | 15811-15812 |
| transcription termination factor, RNA polymerase II | TTF2 | 15813-15814 |
| tubby bipartite transcription factor | TUB | 15815 |
| twist family bHLH transcription factor 1 | TWIST1 | 15816 |
| twist family bHLH transcription factor 2 | TWIST2 | 15817 |
| upstream binding protein 1 (LBP-1a) | UBP1 | 15818 |
| upstream binding transcription factor, RNA | UBTF | 15819 |
| polymerase I | | |
| upstream binding transcription factor, RNA | UBTFL1 | 15820 |
| polymerase I-like 1 | | |
| upstream binding transcription factor, RNA | UBTFL6 | 15821 |
| polymerase I-like 6 (pseudogene) | | |
| UNC homeobox | UNCX | 15822 |
| unkempt family zinc finger | UNK | 15823 |
| unkempt family like zinc finger | UNKL | 15824 |
| upstream transcription factor 1 | USF1 | 15825 |
| upstream transcription factor 2, c-fos interacting | USF2 | 15826 |
| upstream transcription factor family member 3 | USF3 | 15827 |
| undifferentiated embryonic cell transcription factor 1 | UTF1 | 15828 |
| ventral anterior homeobox 1 | VAX1 | 15829 |
| ventral anterior homeobox 2 | VAX2 | 15830 |
| vitamin D (1,25-dihydroxyvitamin D3) receptor | VDR | 15831 |
| VENT homeobox | VENTX | 15832 |
| vascular endothelial zinc finger 1 | VEZF1 | 15833 |
| visual system homeobox 1 | VSX1 | 15834 |
| visual system homeobox 2 | VSX2 | 15835 |
| WD repeat and HMG-box DNA binding protein 1 | WDHD1 | 15836 |
| Wolf-Hirschhorn syndrome candidate 1 | WHSC1 | 15837 |
| widely interspaced zinc finger motifs | WIZ | 15838 |
| Wilms tumor 1 | WT1 | 15839 |
| X-box binding protein 1 | XBP1 | 15840 |
| Y-box binding protein 1 | YBX1 | 15841 |
| Y-box binding protein 2 | YBX2 | 15842 |
| Y-box binding protein 3 | YBX3 | 15843 |
| YEATS domain containing 2 | YEATS2 | 15844 |
| YEATS domain containing 4 | YEATS4 | 15845 |
| YY1 transcription factor | YY1 | 15846 |
| YY2 transcription factor | YY2 | 15847 |
| zinc finger BED-type containing 1 | ZBED1 | 15848 |
| zinc finger BED-type containing 2 | ZBED2 | 15849 |
| zinc finger BED-type containing 3 | ZBED3 | 15850 |
| zinc finger BED-type containing 4 | ZBED4 | 15851 |
| zinc finger BED-type containing 5 | ZBED5 | 15852 |
| zinc finger, BED-type containing 6 | ZBED6 | 15853 |
| Z-DNA binding protein 1 | ZBP1 | 15854-15855 |
| zinc finger and BTB domain containing 1 | ZBTB1 | 15856 |
| zinc finger and BTB domain containing 10 | ZBTB10 | 15857 |
| zinc finger and BTB domain containing 11 | ZBTB11 | 15858 |
| zinc finger and BTB domain containing 12 | ZBTB12 | 15859 |
| zinc finger and BTB domain containing 14 | ZBTB14 | 15860 |
| zinc finger and BTB domain containing 16 | ZBTB16 | 15861 |
| zinc finger and BTB domain containing 17 | ZBTB17 | 15862 |
| zinc finger and BTB domain containing 18 | ZBTB18 | 15863 |
| zinc finger and BTB domain containing 2 | ZBTB2 | 15864 |
| zinc finger and BTB domain containing 20 | ZBTB20 | 15865 |
| zinc finger and BTB domain containing 21 | ZBTB21 | 15866 |
| zinc finger and BTB domain containing 22 | ZBTB22 | 15867 |
| zinc finger and BTB domain containing 24 | ZBTB24 | 15868 |
| zinc finger and BTB domain containing 25 | ZBTB25 | 15869 |
| zinc finger and BTB domain containing 26 | ZBTB26 | 15870 |
| zinc finger and BTB domain containing 3 | ZBTB3 | 15871 |
| zinc finger and BTB domain containing 32 | ZBTB32 | 15872 |
| zinc finger and BTB domain containing 33 | ZBTB33 | 15873 |
| zinc finger and BTB domain containing 34 | ZBTB34 | 15874 |
| zinc finger and BTB domain containing 37 | ZBTB37 | 15875 |
| zinc finger and BTB domain containing 38 | ZBTB38 | 15876 |
| zinc finger and BTB domain containing 39 | ZBTB39 | 15877 |
| zinc finger and BTB domain containing 4 | ZBTB4 | 15878 |
| zinc finger and BTB domain containing 40 | ZBTB40 | 15879 |
| zinc finger and BTB domain containing 41 | ZBTB41 | 15880 |
| zinc finger and BTB domain containing 42 | ZBTB42 | 15881 |
| zinc finger and BTB domain containing 43 | ZBTB43 | 15882 |
| zinc finger and BTB domain containing 44 | ZBTB44 | 15883 |
| zinc finger and BTB domain containing 45 | ZBTB45 | 15884 |
| zinc finger and BTB domain containing 46 | ZBTB46 | 15885 |
| zinc finger and BTB domain containing 47 | ZBTB47 | 15886 |
| zinc finger and BTB domain containing 48 | ZBTB48 | 15887 |
| zinc finger and BTB domain containing 49 | ZBTB49 | 15888 |
| zinc finger and BTB domain containing 5 | ZBTB5 | 15889 |
| zinc finger and BTB domain containing 6 | ZBTB6 | 15890 |
| zinc finger and BTB domain containing 7A | ZBTB7A | 15891 |
| zinc finger and BTB domain containing 7B | ZBTB7B | 15892 |
| zinc finger and BTB domain containing 7C | ZBTB7C | 15893 |
| zinc finger and BTB domain containing 8A | ZBTB8A | 15894 |
| zinc finger and BTB domain containing 9 | ZBTB9 | 15895 |
| zinc finger CCCH-type containing 10 | ZC3H10 | 15896 |
| zinc finger CCCH-type containing 11A | ZC3H11A | 15897 |
| zinc finger CCCH-type containing 12A | ZC3H12A | 15898 |
| zinc finger CCCH-type containing 12B | ZC3H12B | 15899 |
| zinc finger CCCH-type containing 13 | ZC3H13 | 15900 |
| zinc finger CCCH-type containing 14 | ZC3H14 | 15901 |
| zinc finger CCCH-type containing 15 | ZC3H15 | 15902 |
| zinc finger CCCH-type containing 18 | ZC3H18 | 15903 |
| zinc finger CCCH-type containing 3 | ZC3H3 | 15904 |
| zinc finger CCCH-type containing 4 | ZC3H4 | 15905 |
| zinc finger CCCH-type containing 6 | ZC3H6 | 15906 |
| zinc finger CCCH-type containing 7A | ZC3H7A | 15907 |
| zinc finger CCCH-type containing 7B | ZC3H7B | 15908 |
| zinc finger CCCH-type containing 8 | ZC3H8 | 15909 |
| zinc finger CCHC-type containing 11 | ZCCHC11 | 15910 |
| zinc finger CCHC-type containing 6 | ZCCHC6 | 15911 |
| zinc finger E-box binding homeobox 1 | ZEB1 | 15912 |
| zinc finger E-box binding homeobox 2 | ZEB2 | 15913 |
| zinc finger and AT-hook domain containing | ZFAT | 15914 |
| zinc finger homeobox 2 | ZFHX2 | 15915 |
| zinc finger homeobox 3 | ZFHX3 | 15916 |
| zinc finger homeobox 4 | ZFHX4 | 15917 |
| ZFP1 zinc finger protein | ZFP1 | 15918 |
| ZFP14 zinc finger protein | ZFP14 | 15919 |
| ZFP2 zinc finger protein | ZFP2 | 15920 |
| ZFP28 zinc finger protein | ZFP28 | 15921 |
| ZFP3 zinc finger protein | ZFP3 | 15922 |
| ZFP30 zinc finger protein | ZFP30 | 15923 |
| ZFP36 ring finger protein-like 1 | ZFP36L1 | 15924 |
| ZFP36 ring finger protein-like 2 | ZFP36L2 | 15925 |
| ZFP37 zinc finger protein | ZFP37 | 15926 |
| ZFP41 zinc finger protein | ZFP41 | 15927 |
| ZFP42 zinc finger protein | ZFP42 | 15928 |
| ZFP57 zinc finger protein | ZFP57 | 15929 |
| ZFP62 zinc finger protein | ZFP62 | 15930 |
| ZFP64 zinc finger protein | ZFP64 | 15931 |
| ZFP69 zinc finger protein | ZFP69 | 15932-15933 |
| ZFP69 zinc finger protein B | ZFP69B | 15934 |
| ZFP82 zinc finger protein | ZFP82 | 15935 |
| ZFP90 zinc finger protein | ZFP90 | 15936 |
| ZFP91 zinc finger protein | ZFP91 | 15937 |
| ZFP92 zinc finger protein | ZFP92 | 15938 |
| zinc finger protein, FOG family member 1 | ZFPM1 | 15939 |
| zinc finger protein, FOG family member 2 | ZFPM2 | 15940 |
| zinc finger protein, X-linked | ZFX | 15941 |
| zinc finger protein, Y-linked | ZFY | 15942 |
| zinc finger, FYVE domain containing 26 | ZFYVE26 | 15943 |
| zinc finger, GATA-like protein 1 | ZGLP1 | 15944 |
| zinc finger CCCH-type and G-patch domain | ZGPAT | 15945 |
| containing | | |
| zinc fingers and homeoboxes 1 | ZHX1 | 15946 |
| zinc fingers and homeoboxes 2 | ZHX2 | 15947 |
| zinc fingers and homeoboxes 3 | ZHX3 | 15948 |
| Zic family member 1 | ZIC1 | 15949 |
| Zic family member 2 | ZIC2 | 15950 |
| Zic family member 3 | ZIC3 | 15951 |
| Zic family member 4 | ZIC4 | 15952 |
| Zic family member 5 | ZIC5 | 15953 |
| zinc finger protein interacting with K protein 1 | ZIK1 | 15954 |
| zinc finger, imprinted 2 | ZIM2 | 15955 |
| zinc finger, imprinted 3 | ZIM3 | 15956 |
| zinc finger with KRAB and SCAN domains 1 | ZKSCAN1 | 15957 |
| zinc finger with KRAB and SCAN domains 2 | ZKSCAN2 | 15958 |
| zinc finger with KRAB and SCAN domains 3 | ZKSCAN3 | 15959 |
| zinc finger with KRAB and SCAN domains 4 | ZKSCAN4 | 15960 |
| zinc finger with KRAB and SCAN domains 5 | ZKSCAN5 | 15961 |
| zinc finger with KRAB and SCAN domains 7 | ZKSCAN7 | 15962 |
| zinc finger with KRAB and SCAN domains 8 | ZKSCAN8 | 15963 |
| zinc finger matrin-type 1 | ZMAT1 | 15964 |
| zinc finger matrin-type 2 | ZMAT2 | 15965 |
| zinc finger matrin-type 3 | ZMAT3 | 15966 |
| zinc finger matrin-type 4 | ZMAT4 | 15967 |
| zinc finger matrin-type 5 | ZMAT5 | 15968 |
| zinc finger protein 10 | ZNF10 | 15969 |
| zinc finger protein 100 | ZNF100 | 15970 |
| zinc finger protein 101 | ZNF101 | 15971 |
| zinc finger protein 106 | ZNF106 | 15972 |
| zinc finger protein 107 | ZNF107 | 15973 |
| zinc finger protein 112 | ZNF112 | 15974 |
| zinc finger protein 114 | ZNF114 | 15975 |
| zinc finger protein 117 | ZNF117 | 15976 |
| zinc finger protein 12 | ZNF12 | 15977 |
| zinc finger protein 121 | ZNF121 | 15978 |
| zinc finger protein 124 | ZNF124 | 15979 |
| zinc finger protein 131 | ZNF131 | 15980 |
| zinc finger protein 132 | ZNF132 | 15981 |
| zinc finger protein 133 | ZNF133 | 15982 |
| zinc finger protein 134 | ZNF134 | 15983 |
| zinc finger protein 135 | ZNF135 | 15984 |
| zinc finger protein 136 | ZNF136 | 15985 |
| zinc finger protein 137, pseudogene | ZNF137P | 15986 |
| zinc finger protein 138 | ZNF138 | 15987 |
| zinc finger protein 14 | ZNF14 | 15988 |
| zinc finger protein 140 | ZNF140 | 15989 |
| zinc finger protein 141 | ZNF141 | 15990 |
| zinc finger protein 142 | ZNF142 | 15991 |
| zinc finger protein 143 | ZNF143 | 15992 |
| zinc finger protein 146 | ZNF146 | 15993 |
| zinc finger protein 148 | ZNF148 | 15994 |
| zinc finger protein 154 | ZNF154 | 15995 |
| zinc finger protein 155 | ZNF155 | 15996 |
| zinc finger protein 157 | ZNF157 | 15997 |
| zinc finger protein 16 | ZNF16 | 15998 |
| zinc finger protein 160 | ZNF160 | 15999 |
| zinc finger protein 165 | ZNF165 | 16000 |
| zinc finger protein 169 | ZNF169 | 16001 |
| zinc finger protein 17 | ZNF17 | 16002 |
| zinc finger protein 174 | ZNF174 | 16003 |
| zinc finger protein 175 | ZNF175 | 16004 |
| zinc finger protein 18 | ZNF18 | 16005 |
| zinc finger protein 180 | ZNF180 | 16006 |
| zinc finger protein 181 | ZNF181 | 16007 |
| zinc finger protein 182 | ZNF182 | 16008 |
| zinc finger protein 184 | ZNF184 | 16009 |
| zinc finger protein 189 | ZNF189 | 16010 |
| zinc finger protein 19 | ZNF19 | 16011 |
| zinc finger protein 195 | ZNF195 | 16012 |
| zinc finger protein 197 | ZNF197 | 16013 |
| zinc finger protein 2 | ZNF2 | 16014 |
| zinc finger protein 20 | ZNF20 | 16015-16016 |
| zinc finger protein 200 | ZNF200 | 16017 |
| zinc finger protein 202 | ZNF202 | 16018 |
| zinc finger protein 205 | ZNF205 | 16019 |
| zinc finger protein 207 | ZNF207 | 16020 |
| zinc finger protein 208 | ZNF208 | 16021 |
| zinc finger protein 211 | ZNF211 | 16022 |
| zinc finger protein 212 | ZNF212 | 16023 |
| zinc finger protein 213 | ZNF213 | 16024 |
| zinc finger protein 214 | ZNF214 | 16025 |
| zinc finger protein 215 | ZNF215 | 16026 |
| zinc finger protein 217 | ZNF217 | 16027 |
| zinc finger protein 219 | ZNF219 | 16028 |
| zinc finger protein 22 | ZNF22 | 16029 |
| zinc finger protein 221 | ZNF221 | 16030 |
| zinc finger protein 223 | ZNF223 | 16031 |
| zinc finger protein 224 | ZNF224 | 16032 |
| zinc finger protein 225 | ZNF225 | 16033-16034 |
| zinc finger protein 226 | ZNF226 | 16035 |
| zinc finger protein 227 | ZNF227 | 16036 |
| zinc finger protein 229 | ZNF229 | 16037 |
| zinc finger protein 23 | ZNF23 | 16038 |
| zinc finger protein 230 | ZNF230 | 16039-16040 |
| zinc finger protein 232 | ZNF232 | 16041 |
| zinc finger protein 233 | ZNF233 | 16042-16043 |
| zinc finger protein 234 | ZNF234 | 16044 |
| zinc finger protein 235 | ZNF235 | 16045 |
| zinc finger protein 236 | ZNF236 | 16046 |
| zinc finger protein 239 | ZNF239 | 16047 |
| zinc finger protein 24 | ZNF24 | 16048 |
| zinc finger protein 248 | ZNF248 | 16049 |
| zinc finger protein 25 | ZNF25 | 16050 |
| zinc finger protein 250 | ZNF250 | 16051 |
| zinc finger protein 251 | ZNF251 | 16052 |
| zinc finger protein 252, pseudogene | ZNF252P | 16053 |
| zinc finger protein 253 | ZNF253 | 16054 |
| zinc finger protein 254 | ZNF254 | 16055 |
| zinc finger protein 256 | ZNF256 | 16056 |
| zinc finger protein 257 | ZNF257 | 16057 |
| zinc finger protein 26 | ZNF26 | 16058 |
| zinc finger protein 260 | ZNF260 | 16059 |
| zinc finger protein 263 | ZNF263 | 16060 |
| zinc finger protein 264 | ZNF264 | 16061 |
| zinc finger protein 266 | ZNF266 | 16062 |
| zinc finger protein 267 | ZNF267 | 16063 |
| zinc finger protein 268 | ZNF268 | 16064 |
| zinc finger protein 273 | ZNF273 | 16065 |
| zinc finger protein 274 | ZNF274 | 16066 |
| zinc finger protein 275 | ZNF275 | 16067 |
| zinc finger protein 276 | ZNF276 | 16068 |
| zinc finger protein 277 | ZNF277 | 16069 |
| zinc finger protein 28 | ZNF28 | 16070 |
| zinc finger protein 280A | ZNF280A | 16071 |
| zinc finger protein 280B | ZNF280B | 16072 |
| zinc finger protein 280C | ZNF280C | 16073 |
| zinc finger protein 280D | ZNF280D | 16074 |
| zinc finger protein 281 | ZNF281 | 16075 |
| zinc finger protein 282 | ZNF282 | 16076 |
| zinc finger protein 283 | ZNF283 | 16077 |
| zinc finger protein 284 | ZNF284 | 16078 |
| zinc finger protein 285 | ZNF285 | 16079 |
| zinc finger protein 286A | ZNF286A | 16080 |
| zinc finger protein 286B | ZNF286B | 16081 |
| zinc finger protein 287 | ZNF287 | 16082 |
| zinc finger protein 292 | ZNF292 | 16083 |
| zinc finger protein 296 | ZNF296 | 16084 |
| zinc finger protein 3 | ZNF3 | 16085 |
| zinc finger protein 30 | ZNF30 | 16086 |
| zinc finger protein 300 | ZNF300 | 16087 |
| zinc finger protein 302 | ZNF302 | 16088 |
| zinc finger protein 304 | ZNF304 | 16089 |
| zinc finger protein 311 | ZNF311 | 16090 |
| zinc finger protein 316 | ZNF316 | 16091 |
| zinc finger protein 317 | ZNF317 | 16092 |
| zinc finger protein 318 | ZNF318 | 16093 |
| zinc finger protein 319 | ZNF319 | 16094 |
| zinc finger protein 32 | ZNF32 | 16095 |
| zinc finger protein 320 | ZNF320 | 16096 |
| zinc finger protein 322 | ZNF322 | 16097 |
| zinc finger protein 324 | ZNF324 | 16098 |
| zinc finger protein 324B | ZNF324B | 16099 |
| zinc finger protein 326 | ZNF326 | 16100 |
| zinc finger protein 329 | ZNF329 | 16101 |
| zinc finger protein 331 | ZNF331 | 16102 |
| zinc finger protein 333 | ZNF333 | 16103 |
| zinc finger protein 334 | ZNF334 | 16104 |
| zinc finger protein 335 | ZNF335 | 16105 |
| zinc finger protein 337 | ZNF337 | 16106 |
| zinc finger protein 33A | ZNF33A | 16107 |
| zinc finger protein 33B | ZNF33B | 16108 |
| zinc finger protein 34 | ZNF34 | 16109 |
| zinc finger protein 341 | ZNF341 | 16110 |
| zinc finger protein 343 | ZNF343 | 16111 |
| zinc finger protein 345 | ZNF345 | 16112 |
| zinc finger protein 346 | ZNF346 | 16113 |
| zinc finger protein 347 | ZNF347 | 16114 |
| zinc finger protein 35 | ZNF35 | 16115 |
| zinc finger protein 350 | ZNF350 | 16116 |
| zinc finger protein 354A | ZNF354A | 16117 |
| zinc finger protein 354B | ZNF354B | 16118 |
| zinc finger protein 354C | ZNF354C | 16119 |
| zinc finger protein 355, pseudogene | ZNF355P | 16120 |
| zinc finger protein 358 | ZNF358 | 16121 |
| zinc finger protein 362 | ZNF362 | 16122 |
| zinc finger protein 365 | ZNF365 | 16123-16124 |
| zinc finger protein 366 | ZNF366 | 16125 |
| zinc finger protein 367 | ZNF367 | 16126 |
| zinc finger protein 37A | ZNF37A | 16127 |
| zinc finger protein 382 | ZNF382 | 16128 |
| zinc finger protein 383 | ZNF383 | 16129 |
| zinc finger protein 384 | ZNF384 | 16130 |
| zinc finger protein 385A | ZNF385A | 16131 |
| zinc finger protein 385B | ZNF385B | 16132 |
| zinc finger protein 385C | ZNF385C | 16133 |
| zinc finger protein 385D | ZNF385D | 16134 |
| zinc finger protein 391 | ZNF391 | 16135 |
| zinc finger protein 394 | ZNF394 | 16136 |
| zinc finger protein 395 | ZNF395 | 16137 |
| zinc finger protein 396 | ZNF396 | 16138 |
| zinc finger protein 397 | ZNF397 | 16139 |
| zinc finger protein 398 | ZNF398 | 16140 |
| zinc finger protein 404 | ZNF404 | 16141 |
| zinc finger protein 407 | ZNF407 | 16142 |
| zinc finger protein 408 | ZNF408 | 16143 |
| zinc finger protein 41 | ZNF41 | 16144 |
| zinc finger protein 410 | ZNF410 | 16145 |
| zinc finger protein 414 | ZNF414 | 16146 |
| zinc finger protein 415 | ZNF415 | 16147 |
| zinc finger protein 416 | ZNF416 | 16148 |
| zinc finger protein 417 | ZNF417 | 16149 |
| zinc finger protein 418 | ZNF418 | 16150 |
| zinc finger protein 419 | ZNF419 | 16151 |
| zinc finger protein 420 | ZNF420 | 16152 |
| zinc finger protein 423 | ZNF423 | 16153 |
| zinc finger protein 425 | ZNF425 | 16154 |
| zinc finger protein 426 | ZNF426 | 16155 |
| zinc finger protein 428 | ZNF428 | 16156 |
| zinc finger protein 429 | ZNF429 | 16157 |
| zinc finger protein 43 | ZNF43 | 16158 |
| zinc finger protein 430 | ZNF430 | 16159 |
| zinc finger protein 431 | ZNF431 | 16160 |
| zinc finger protein 432 | ZNF432 | 16161 |
| zinc finger protein 433 | ZNF433 | 16162 |
| zinc finger protein 436 | ZNF436 | 16163 |
| zinc finger protein 438 | ZNF438 | 16164 |
| zinc finger protein 439 | ZNF439 | 16165 |
| zinc finger protein 44 | ZNF44 | 16166 |
| zinc finger protein 440 | ZNF440 | 16167 |
| zinc finger protein 441 | ZNF441 | 16168 |
| zinc finger protein 442 | ZNF442 | 16169 |
| zinc finger protein 443 | ZNF443 | 16170 |
| zinc finger protein 444 | ZNF444 | 16171 |
| zinc finger protein 445 | ZNF445 | 16172 |
| zinc finger protein 446 | ZNF446 | 16173 |
| zinc finger protein 449 | ZNF449 | 16174 |
| zinc finger protein 45 | ZNF45 | 16175 |
| zinc finger protein 451 | ZNF451 | 16176 |
| zinc finger protein 454 | ZNF454 | 16177 |
| zinc finger protein 460 | ZNF460 | 16178 |
| zinc finger protein 461 | ZNF461 | 16179 |
| zinc finger protein 462 | ZNF462 | 16180 |
| zinc finger protein 467 | ZNF467 | 16181 |
| zinc finger protein 468 | ZNF468 | 16182 |
| zinc finger protein 469 | ZNF469 | 16183 |
| zinc finger protein 470 | ZNF470 | 16184 |
| zinc finger protein 471 | ZNF471 | 16185 |
| zinc finger protein 473 | ZNF473 | 16186 |
| zinc finger protein 474 | ZNF474 | 16187-16188 |
| zinc finger protein 479 | ZNF479 | 16189 |
| zinc finger protein 48 | ZNF48 | 16190 |
| zinc finger protein 480 | ZNF480 | 16191 |
| zinc finger protein 483 | ZNF483 | 16192 |
| zinc finger protein 484 | ZNF484 | 16193 |
| zinc finger protein 485 | ZNF485 | 16194 |
| zinc finger protein 486 | ZNF486 | 16195 |
| zinc finger protein 487 | ZNF487 | 16196 |
| zinc finger protein 488 | ZNF488 | 16197 |
| zinc finger protein 490 | ZNF490 | 16198 |
| zinc finger protein 491 | ZNF491 | 16199 |
| zinc finger protein 492 | ZNF492 | 16200 |
| zinc finger protein 493 | ZNF493 | 16201 |
| zinc finger protein 496 | ZNF496 | 16202 |
| zinc finger protein 497 | ZNF497 | 16203 |
| zinc finger protein 500 | ZNF500 | 16204 |
| zinc finger protein 501 | ZNF501 | 16205 |
| zinc finger protein 502 | ZNF502 | 16206 |
| zinc finger protein 503 | ZNF503 | 16207 |
| zinc finger protein 506 | ZNF506 | 16208 |
| zinc finger protein 507 | ZNF507 | 16209 |
| zinc finger protein 510 | ZNF510 | 16210 |
| zinc finger protein 511 | ZNF511 | 16211 |
| zinc finger protein 512 | ZNF512 | 16212 |
| zinc finger protein 512B | ZNF512B | 16213 |
| zinc finger protein 513 | ZNF513 | 16214 |
| zinc finger protein 514 | ZNF514 | 16215 |
| zinc finger protein 516 | ZNF516 | 16216 |
| zinc finger protein 517 | ZNF517 | 16217 |
| zinc finger protein 518A | ZNF518A | 16218 |
| zinc finger protein 518B | ZNF518B | 16219 |
| zinc finger protein 519 | ZNF519 | 16220 |
| zinc finger protein 521 | ZNF521 | 16221 |
| zinc finger protein 524 | ZNF524 | 16222 |
| zinc finger protein 526 | ZNF526 | 16223 |
| zinc finger protein 527 | ZNF527 | 16224 |
| zinc finger protein 528 | ZNF528 | 16225 |
| zinc finger protein 529 | ZNF529 | 16226 |
| zinc finger protein 530 | ZNF530 | 16227 |
| zinc finger protein 532 | ZNF532 | 16228 |
| zinc finger protein 534 | ZNF534 | 16229 |
| zinc finger protein 536 | ZNF536 | 16230 |
| zinc finger protein 540 | ZNF540 | 16231 |
| zinc finger protein 541 | ZNF541 | 16232 |
| zinc finger protein 542, pseudogene | ZNF542P | 16233 |
| zinc finger protein 543 | ZNF543 | 16234 |
| zinc finger protein 544 | ZNF544 | 16235 |
| zinc finger protein 546 | ZNF546 | 16236 |
| zinc finger protein 547 | ZNF547 | 16237 |
| zinc finger protein 548 | ZNF548 | 16238 |
| zinc finger protein 549 | ZNF549 | 16239 |
| zinc finger protein 550 | ZNF550 | 16240 |
| zinc finger protein 552 | ZNF552 | 16241 |
| zinc finger protein 554 | ZNF554 | 16242 |
| zinc finger protein 555 | ZNF555 | 16243 |
| zinc finger protein 556 | ZNF556 | 16244 |
| zinc finger protein 557 | ZNF557 | 16245 |
| zinc finger protein 558 | ZNF558 | 16246 |
| zinc finger protein 559 | ZNF559 | 16247 |
| zinc finger protein 56 | ZNF56 | 16248 |
| zinc finger protein 560 | ZNF560 | 16249 |
| zinc finger protein 561 | ZNF561 | 16250 |
| zinc finger protein 562 | ZNF562 | 16251 |
| zinc finger protein 563 | ZNF563 | 16252 |
| zinc finger protein 564 | ZNF564 | 16253 |
| zinc finger protein 565 | ZNF565 | 16254 |
| zinc finger protein 566 | ZNF566 | 16255 |
| zinc finger protein 567 | ZNF567 | 16256 |
| zinc finger protein 568 | ZNF568 | 16257 |
| zinc finger protein 569 | ZNF569 | 16258 |
| zinc finger protein 57 | ZNF57 | 16259 |
| zinc finger protein 570 | ZNF570 | 16260 |
| zinc finger protein 571 | ZNF571 | 16261 |
| zinc finger protein 572 | ZNF572 | 16262 |
| zinc finger protein 573 | ZNF573 | 16263 |
| zinc finger protein 574 | ZNF574 | 16264 |
| zinc finger protein 575 | ZNF575 | 16265 |
| zinc finger protein 576 | ZNF576 | 16266-16267 |
| zinc finger protein 577 | ZNF577 | 16268 |
| zinc finger protein 578 | ZNF578 | 16269 |
| zinc finger protein 579 | ZNF579 | 16270 |
| zinc finger protein 580 | ZNF580 | 16271 |
| zinc finger protein 581 | ZNF581 | 16272 |
| zinc finger protein 582 | ZNF582 | 16273 |
| zinc finger protein 583 | ZNF583 | 16274 |
| zinc finger protein 584 | ZNF584 | 16275 |
| zinc finger protein 585A | ZNF585A | 16276 |
| zinc finger protein 585B | ZNF585B | 16277 |
| zinc finger protein 586 | ZNF586 | 16278 |
| zinc finger protein 587 | ZNF587 | 16279 |
| zinc finger protein 589 | ZNF589 | 16280 |
| zinc finger protein 592 | ZNF592 | 16281 |
| zinc finger protein 593 | ZNF593 | 16282 |
| zinc finger protein 594 | ZNF594 | 16283 |
| zinc finger protein 595 | ZNF595 | 16284 |
| zinc finger protein 596 | ZNF596 | 16285 |
| zinc finger protein 597 | ZNF597 | 16286 |
| zinc finger protein 598 | ZNF598 | 16287 |
| zinc finger protein 599 | ZNF599 | 16288 |
| zinc finger protein 600 | ZNF600 | 16289 |
| zinc finger protein 605 | ZNF605 | 16290 |
| zinc finger protein 606 | ZNF606 | 16291 |
| zinc finger protein 607 | ZNF607 | 16292 |
| zinc finger protein 608 | ZNF608 | 16293 |
| zinc finger protein 609 | ZNF609 | 16294 |
| zinc finger protein 610 | ZNF610 | 16295 |
| zinc finger protein 611 | ZNF611 | 16296 |
| zinc finger protein 613 | ZNF613 | 16297 |
| zinc finger protein 614 | ZNF614 | 16298 |
| zinc finger protein 615 | ZNF615 | 16299 |
| zinc finger protein 616 | ZNF616 | 16300 |
| zinc finger protein 618 | ZNF618 | 16301 |
| zinc finger protein 619 | ZNF619 | 16302 |
| zinc finger protein 620 | ZNF620 | 16303 |
| zinc finger protein 621 | ZNF621 | 16304 |
| zinc finger protein 622 | ZNF622 | 16305 |
| zinc finger protein 623 | ZNF623 | 16306 |
| zinc finger protein 624 | ZNF624 | 16307 |
| zinc finger protein 625 | ZNF625 | 16308 |
| zinc finger protein 626 | ZNF626 | 16309 |
| zinc finger protein 627 | ZNF627 | 16310 |
| zinc finger protein 628 | ZNF628 | 16311 |
| zinc finger protein 629 | ZNF629 | 16312 |
| zinc finger protein 639 | ZNF639 | 16313 |
| zinc finger protein 641 | ZNF641 | 16314 |
| zinc finger protein 644 | ZNF644 | 16315 |
| zinc finger protein 645 | ZNF645 | 16316 |
| zinc finger protein 646 | ZNF646 | 16317 |
| zinc finger protein 648 | ZNF648 | 16318 |
| zinc finger protein 649 | ZNF649 | 16319 |
| zinc finger protein 652 | ZNF652 | 16320 |
| zinc finger protein 653 | ZNF653 | 16321 |
| zinc finger protein 654 | ZNF654 | 16322 |
| zinc finger protein 655 | ZNF655 | 16323 |
| zinc finger protein 658 | ZNF658 | 16324 |
| zinc finger protein 658B (pseudogene) | ZNF658B | 16325 |
| zinc finger protein 66 | ZNF66 | 16326 |
| zinc finger protein 660 | ZNF660 | 16327 |
| zinc finger protein 662 | ZNF662 | 16328 |
| zinc finger protein 664 | ZNF664 | 16329 |
| zinc finger protein 665 | ZNF665 | 16330 |
| zinc finger protein 667 | ZNF667 | 16331 |
| zinc finger protein 668 | ZNF668 | 16332 |
| zinc finger protein 669 | ZNF669 | 16333 |
| zinc finger protein 670 | ZNF670 | 16334 |
| zinc finger protein 671 | ZNF671 | 16335 |
| zinc finger protein 672 | ZNF672 | 16336 |
| zinc finger protein 674 | ZNF674 | 16337 |
| zinc finger protein 675 | ZNF675 | 16338 |
| zinc finger protein 676 | ZNF676 | 16339 |
| zinc finger protein 677 | ZNF677 | 16340 |
| zinc finger protein 678 | ZNF678 | 16341 |
| zinc finger protein 679 | ZNF679 | 16342 |
| zinc finger protein 680 | ZNF680 | 16343 |
| zinc finger protein 681 | ZNF681 | 16344 |
| zinc finger protein 682 | ZNF682 | 16345 |
| zinc finger protein 683 | ZNF683 | 16346 |
| zinc finger protein 684 | ZNF684 | 16347 |
| zinc finger protein 687 | ZNF687 | 16348 |
| zinc finger protein 688 | ZNF688 | 16349 |
| zinc finger protein 689 | ZNF689 | 16350 |
| zinc finger protein 69 | ZNF69 | 16351 |
| zinc finger protein 691 | ZNF691 | 16352 |
| zinc finger protein 692 | ZNF692 | 16353 |
| zinc finger protein 695 | ZNF695 | 16354 |
| zinc finger protein 696 | ZNF696 | 16355 |
| zinc finger protein 697 | ZNF697 | 16356 |
| zinc finger protein 699 | ZNF699 | 16357 |
| zinc finger protein 7 | ZNF7 | 16358 |
| zinc finger protein 70 | ZNF70 | 16359 |
| zinc finger protein 701 | ZNF701 | 16360 |
| zinc finger protein 702, pseudogene | ZNF702P | 16361 |
| zinc finger protein 703 | ZNF703 | 16362 |
| zinc finger protein 704 | ZNF704 | 16363 |
| zinc finger protein 705A | ZNF705A | 16364 |
| zinc finger protein 705D | ZNF705D | 16365 |
| zinc finger protein 705E | ZNF705E | 16366 |
| zinc finger protein 705G | ZNF705G | 16367 |
| zinc finger protein 706 | ZNF706 | 16368 |
| zinc finger protein 707 | ZNF707 | 16369 |
| zinc finger protein 708 | ZNF708 | 16370 |
| zinc finger protein 709 | ZNF709 | 16371 |
| zinc finger protein 71 | ZNF71 | 16372 |
| zinc finger protein 710 | ZNF710 | 16373 |
| zinc finger protein 711 | ZNF711 | 16374 |
| zinc finger protein 713 | ZNF713 | 16375 |
| zinc finger protein 714 | ZNF714 | 16376 |
| zinc finger protein 716 | ZNF716 | 16377 |
| zinc finger protein 717 | ZNF717 | 16378 |
| zinc finger protein 718 | ZNF718 | 16379 |
| zinc finger protein 720 | ZNF720 | 16380 |
| zinc finger protein 721 | ZNF721 | 16381 |
| zinc finger protein 724, pseudogene | ZNF724P | 16382 |
| zinc finger protein 726 | ZNF726 | 16383 |
| zinc finger protein 727 | ZNF727 | 16384 |
| zinc finger protein 729 | ZNF729 | 16385 |
| zinc finger protein 730 | ZNF730 | 16386 |
| zinc finger protein 732 | ZNF732 | 16387 |
| zinc finger protein 735 | ZNF735 | 16388 |
| zinc finger protein 737 | ZNF737 | 16389 |
| zinc finger protein 74 | ZNF74 | 16390 |
| zinc finger protein 740 | ZNF740 | 16391 |
| zinc finger protein 746 | ZNF746 | 16392 |
| zinc finger protein 747 | ZNF747 | 16393 |
| zinc finger protein 749 | ZNF749 | 16394 |
| zinc finger protein 750 | ZNF750 | 16395 |
| zinc finger protein 75a | ZNF75A | 16396 |
| zinc finger protein 75D | ZNF75D | 16397 |
| zinc finger protein 76 | ZNF76 | 16398 |
| zinc finger protein 761 | ZNF761 | 16399 |
| zinc finger protein 763 | ZNF763 | 16400 |
| zinc finger protein 764 | ZNF764 | 16401 |
| zinc finger protein 765 | ZNF765 | 16402 |
| zinc finger protein 766 | ZNF766 | 16403 |
| zinc finger protein 768 | ZNF768 | 16404 |
| zinc finger protein 77 | ZNF77 | 16405 |
| zinc finger protein 770 | ZNF770 | 16406 |
| zinc finger protein 771 | ZNF771 | 16407 |
| zinc finger protein 772 | ZNF772 | 16408 |
| zinc finger protein 773 | ZNF773 | 16409 |
| zinc finger protein 774 | ZNF774 | 16410 |
| zinc finger protein 775 | ZNF775 | 16411 |
| zinc finger protein 776 | ZNF776 | 16412 |
| zinc finger protein 777 | ZNF777 | 16413 |
| zinc finger protein 778 | ZNF778 | 16414 |
| zinc finger protein 780A | ZNF780A | 16415 |
| zinc finger protein 780B | ZNF780B | 16416 |
| zinc finger protein 781 | ZNF781 | 16417 |
| zinc finger protein 782 | ZNF782 | 16418 |
| zinc finger family member 783 | ZNF783 | 16419 |
| zinc finger protein 784 | ZNF784 | 16420 |
| zinc finger protein 785 | ZNF785 | 16421 |
| zinc finger protein 786 | ZNF786 | 16422 |
| zinc finger protein 787 | ZNF787 | 16423 |
| zinc finger family member 788 | ZNF788 | 16424 |
| zinc finger protein 789 | ZNF789 | 16425 |
| zinc finger protein 79 | ZNF79 | 16426 |
| zinc finger protein 790 | ZNF790 | 16427 |
| zinc finger protein 791 | ZNF791 | 16428 |
| zinc finger protein 792 | ZNF792 | 16429 |
| zinc finger protein 793 | ZNF793 | 16430 |
| zinc finger protein 799 | ZNF799 | 16431 |
| zinc finger protein 8 | ZNF8 | 16432 |
| zinc finger protein 80 | ZNF80 | 16433 |
| zinc finger protein 800 | ZNF800 | 16434 |
| zinc finger protein 804A | ZNF804A | 16435 |
| zinc finger protein 804B | ZNF804B | 16436 |
| zinc finger protein 805 | ZNF805 | 16437 |
| zinc finger protein 806 | ZNF806 | 16438 |
| zinc finger protein 808 | ZNF808 | 16439 |
| zinc finger protein 81 | ZNF81 | 16440 |
| zinc finger protein 813 | ZNF813 | 16441 |
| zinc finger protein 814 | ZNF814 | 16442 |
| zinc finger protein 816 | ZNF816 | 16443 |
| zinc finger protein 821 | ZNF821 | 16444 |
| zinc finger protein 823 | ZNF823 | 16445 |
| zinc finger protein 827 | ZNF827 | 16446 |
| zinc finger protein 829 | ZNF829 | 16447 |
| zinc finger protein 83 | ZNF83 | 16448 |
| zinc finger protein 830 | ZNF830 | 16449 |
| zinc finger protein 831 | ZNF831 | 16450 |
| zinc finger protein 833, pseudogene | ZNF833P | 16451 |
| zinc finger protein 835 | ZNF835 | 16452 |
| zinc finger protein 836 | ZNF836 | 16453 |
| zinc finger protein 837 | ZNF837 | 16454 |
| zinc finger protein 839 | ZNF839 | 16455 |
| zinc finger protein 84 | ZNF84 | 16456 |
| zinc finger protein 840, pseudogene | ZNF840P | 16457 |
| zinc finger protein 841 | ZNF841 | 16458 |
| zinc finger protein 843 | ZNF843 | 16459 |
| zinc finger protein 844 | ZNF844 | 16460 |
| zinc finger protein 845 | ZNF845 | 16461 |
| zinc finger protein 846 | ZNF846 | 16462 |
| zinc finger protein 85 | ZNF85 | 16463 |
| zinc finger protein 853 | ZNF853 | 16464 |
| zinc finger protein 860 | ZNF860 | 16465 |
| zinc finger protein 876, pseudogene | ZNF876P | 16466 |
| zinc finger protein 878 | ZNF878 | 16467 |
| zinc finger protein 879 | ZNF879 | 16468 |
| zinc finger protein 880 | ZNF880 | 16469 |
| zinc finger protein 891 | ZNF891 | 16470 |
| zinc finger protein 90 | ZNF90 | 16471 |
| zinc finger protein 91 | ZNF91 | 16472 |
| zinc finger protein 92 | ZNF92 | 16473 |
| zinc finger protein 93 | ZNF93 | 16474 |
| zinc finger protein 98 | ZNF98 | 16475 |
| zinc finger protein 99 | ZNF99 | 16476 |
| zinc finger, NFX1-type containing 1 | ZNFX1 | 16477 |
| zinc finger and SCAN domain containing 1 | ZSCAN1 | 16478 |
| zinc finger and SCAN domain containing 10 | ZSCAN10 | 16479 |
| zinc finger and SCAN domain containing 12 | ZSCAN12 | 16480 |
| zinc finger and SCAN domain containing 16 | ZSCAN16 | 16481 |
| zinc finger and SCAN domain containing 18 | ZSCAN18 | 16482 |
| zinc finger and SCAN domain containing 2 | ZSCAN2 | 16483 |
| zinc finger and SCAN domain containing 20 | ZSCAN20 | 16484 |
| zinc finger and SCAN domain containing 21 | ZSCAN21 | 16485 |
| zinc finger and SCAN domain containing 22 | ZSCAN22 | 16486 |
| zinc finger and SCAN domain containing 23 | ZSCAN23 | 16487 |
| zinc finger and SCAN domain containing 25 | ZSCAN25 | 16488 |
| zinc finger and SCAN domain containing 26 | ZSCAN26 | 16489 |
| zinc finger and SCAN domain containing 29 | ZSCAN29 | 16490 |
| zinc finger and SCAN domain containing 30 | ZSCAN30 | 16491 |
| zinc finger and SCAN domain containing 31 | ZSCAN31 | 16492 |
| zinc finger and SCAN domain containing 32 | ZSCAN32 | 16493 |
| zinc finger and SCAN domain containing 4 | ZSCAN4 | 16494 |
| zinc finger and SCAN domain containing 5A | ZSCAN5A | 16495 |
| zinc finger and SCAN domain containing 5B | ZSCAN5B | 16496 |
| zinc finger and SCAN domain containing 5C, | ZSCAN5CP | 16497 |
| pseudogene | | |
| zinc finger and SCAN domain containing 9 | ZSCAN9 | 16498 |
| zinc finger with UFM1-specific peptidase domain | ZUFSP | 16499 |
| zinc finger, X-linked, duplicated A | ZXDA | 16500 |
| zinc finger, X-linked, duplicated B | ZXDB | 16501 |
| ZXD family zinc finger C | ZXDC | 16502 |
| zinc finger ZZ-type containing 3 | ZZZ3 | 16503 |

In some embodiments, a T-cell of the disclosure is modified to silence or reduce expression of one or more gene(s) encoding a cell death or cell apoptosis receptor to produce an armored T-cell of the disclosure. Interaction of a death receptor and its endogenous ligand results in the initiation of apoptosis. Disruption of an expression, an activity, or an interaction of a cell death and/or cell apoptosis receptor and/or ligand render an armored T-cell of the disclosure less receptive to death signals, consequently, making the armored T cell of the disclosure more efficacious in a tumor environment. An exemplary cell death receptor which may be modified in an armored T cell of the disclosure is Fas (CD95). Exemplary cell death and/or cell apoptosis receptors and ligands of the disclosure include, but are not limited to, the exemplary receptors and ligands provided in Table 4.

TABLE 4
Exemplary Cell Death and/or Cell
Apoptosis Receptors and Ligands.

| Full Name | Abbreviation | SEQ ID NO: |
|---|---|---|
| Cluster of Differentiation 120 | CD120a | 16504-16505 |
| Death receptor 3 | DR3 | 16506 |
| Death receptor 6 | DR6 | 16507 |
| first apoptosis signal (Fas) receptor | Fas | 16508-16509 |
| | (CD95/APO-1) | |
| Fas Ligand | FasL | 16510 |
| cellular tumor antigen p53 | p53 | 16511 |
| Tumor necrosis factor receptor 1 | TNF-R1 | 16512 |
| Tumor necrosis factor receptor 2 | TNF-R2 | 16513 |
| Tumor necrosis factor-related apoptosis- | TRAIL-R1 | 16514 |
| inducing ligand receptor 1 | (DR4) | |
| Tumor necrosis factor-related apoptosis- | TRAIL-R2 | 16515 |
| inducing ligand receptor 2 | (DR5) | |
| Fas-associated protein with death domain | FADD | 16516 |
| Tumor necrosis factor receptor type 1- | TRADD | 16517 |
| associated DEATH domain protein | | |
| Bcl-2-associatcd X protein | Bax | 16518 |
| Bcl-2 homologous killer | BAK | 16519 |
| 14-3-3 protein | 14-3-3 | 16520 |
| B-cell lymphoma 2 | Bcl-2 | 16521 |
| Cytochrome C | CytC | 16522 |
| Second mitochondria-derived activator of | Smac/Diablo | 16523 |
| caspase | | |
| High temperature requirement protein A2 | HTRA2/Omi | 16524 |
| Apoptosis inducing factor | AIF | 16525 |
| Endonuclease G | EXOG | 16526 |
| Caspase 9 | Cas9 | 16527 |
| Caspase 2 | Cas2 | 16528 |
| Caspase 8 | Cas8 | 16529 |
| Caspase 10 | Cas10 | 16530 |
| Caspase 3 | Cas3 | 16531 |
| Caspase 6 | Cas6 | 16532 |
| Caspase 7 | Cas7 | 16533 |
| Tumor Necrosis Factor alpha | TNF-alpha | 16534 |
| TNF-related weak inducer of apoptosis | TWEAK | 16535 |
| TNF-related weak inducer of apoptosis | TWEAK -R | 16536 |
| receptor | | |
| Tumor necrosis factor-related apoptosis- | TRAIL | 16537 |
| inducing ligand | | |
| TNF ligand-related molecule 1 | TL1A | 16538 |
| Receptor-interacting serine/threonine- | RIP1 | 16539 |
| protein kinase 1 | | |
| Cellular inhibitor of apoptosis 1 | cIAP-1 | 16540 |
| TNF receptor-associated factor 2 | TRAF-2 | 16541 |

In some embodiments, a T-cell of the disclosure is modified to silence or reduce expression of one or more gene(s) encoding a metabolic sensing protein to produce an armored T-cell of the disclosure. Disruption to the metabolic sensing of the immunosuppressive tumor microenvironment (characterized by low levels of oxygen, pH, glucose and other molecules) by an armored T-cell of the disclosure leads to extended retention of T-cell function and, consequently, more tumor cells killed per armored T cell. For example, HIF1a and VHL play a role in T-cell function while in a hypoxic environment. An armored T-cell of the disclosure may have silenced or reduced expression of one or more genes encoding HIF1a or VHL. Genes and proteins involved in metabolic sensing include, but are not limited to, the exemplary genes and proteins provided in Table 5.

TABLE 5
Exemplary Metabolic Sensing Genes (and encoded Proteins).

| Full Name | Metabolite | Abbreviation | SEQ ID NO: |
|---|---|---|---|
| hypoxia-inducible factor 1α | Low oxygen | HIF-1α | 16542 |
| von Hippel-Lindau tumor suppressor | Low oxygen | VHL | 16543 |
| Prolyl-hydroxylase domain proteins | High oxygen | PHD proteins | |
| Glucose transporter 1 | glucose | GLUT1 | 16544 |
| Linker of Activated T cells | Amino acid (leucine) | LAT | 16545 |
| CD98 glycoprotein | Amino acid (leucine) | CD98 | 16546 |
| Alanine, serine, cysteine-preferring | Cationic Amino acid | ASCT2/Slc1a5 | 16547 |
| transporter 2 | (glutamine) | | |
| Solute carrier family 7 member 1 | Cationic Amino acids | Slc7a1 | 16548 |
| Solute carrier family 7 member 2 | Cationic Amino acids | Slc7a2 | 16549 |
| Solute carrier family 7 member 3 | Cationic Amino acids | Slc7a3 | 16550 |
| Solute carrier family 7 member 4 | Cationic Amino acids | Slc7a4 | 16551 |
| Solute carrier family 7 member 5 | Glycoprotein | Slc7a5 | 16552 |
| | associated Amino | | |
| | acids | | |
| Solute carrier family 7 member 6 | Glycoprotein | Slc7a6 | 16553 |
| | associated Amino | | |
| | acids | | |
| Solute carrier family 7 member 7 | Glycoprotein | Slc7a7 | 16554 |
| | associated Amino | | |
| | acids | | |
| Solute carrier family 7 member 8 | Glycoprotein | Slc7a8 | 16555 |
| | associated Amino | | |
| | acids | | |
| Solute carrier family 7 member 9 | Glycoprotein | Slc7a9 | 16556 |
| | associated Amino | | |
| | acids | | |
| Solute carrier family 7 member 10 | Glycoprotein | Slc7a10 | 16557 |
| | associated Amino | | |
| | acids | | |
| Solute carrier family 7 member 11 | Glycoprotein | Slc7a11 | 16558 |
| | associated Amino | | |
| | acids | | |
| Solute carrier family 7 member 13 | Glycoprotein | Slc7a13 | 16559 |
| | associated Amino | | |
| | acids | | |
| Solute carrier family 7 member 14 | Cationic Amino acids | Slc7a14 | 16560 |
| Solute carrier family 3 member 2 | Amino acid | Slc3a2 | 16561 |
| Calcium transport protein 2 | Cationic Amino acid | CAT2 | 16562 |
| | (arginine) | | |
| Calcium transport protein 3 | Cationic Amino acid | CAT3 | 16563 |
| | (arginine) | | |
| Calcium transport protein 4 | Cationic Amino acid | CAT4 | 16564 |
| | (arginine) | | |
| Bromodomain adjacent to zinc finger | Amino acid (arginine) | BAZ1B | 16565 |
| domain protein 1B | | | |
| PC4 and SFRS1-interacting protein | Amino acid (arginine) | PSIP1 | 16566 |
| Translin | Amino acid (arginine) | TSN | 16567 |
| G-protein-coupled receptors | Fatty Acid and | GPCRs | |
| | Cholesterol | | |
| T-cell Receptor, subunit alpha | Fatty Acid and | TCR alpha | 16568 |
| | Cholesterol | | |
| T-cell Receptor, subunit beta | Fatty Acid and | TCR beta | 16569 |
| | Cholesterol | | |
| T-cell Receptor, subunit zeta | Fatty Acid and | TCR zeta | 16570 |
| | Cholesterol | | |
| T-cell Receptor, subunit CD3 epsilon | Fatty Acid and | TCR CD3 epsilon | 16571 |
| | Cholesterol | | |
| T-cell Receptor, subunit CD3 | Fatty Acid and | TCR CD3 gamma | 16572 |
| gamma | Cholesterol | | |
| T-cell Receptor, subunit CD3 delta | Fatty Acid and | TCR CD3 delta | 16573 |
| | Cholesterol | | |
| peroxisome proliferator-activated | Fatty Acid and | PPARs | |
| receptors | Cholesterol | | |
| AMP-activated protein kinase | Energy homeostasis | AMPK | 16574-16575 |
| | (intracellular AMP to | | |
| | ATP ratio) | | |
| P2X purinoceptor 7 | Redox homeostasis | P2X7 | 16576 |

In some embodiments, a T-cell of the disclosure is modified to silence or reduce expression of one or more gene(s) encoding proteins that that confer sensitivity to a cancer therapy, including a monoclonal antibody, to produce an armored T-cell of the disclosure. Thus, an armored T-cell of the disclosure can function and may demonstrate superior function or efficacy whilst in the presence of a cancer therapy (e.g. a chemotherapy, a monoclonal antibody therapy, or another anti-tumor treatment). Proteins involved in conferring sensitivity to a cancer therapy include, but are not limited to, the exemplary proteins provided in Table 6.

TABLE 6
Exemplary Proteins that Confer Sensitivity to a Cancer Therapeutic.

| Full Name | Abbreviation | SEQ ID NO: |
|---|---|---|
| Copper-transporting ATPase 2 | ATP7B | 16577 |
| Breakpoint cluster region protein | BCR | 16578 |
| Abelson tyrosine-protein kinase 1 | ABL | 16579 |
| Breast cancer resistance protein | BCRP | 16580 |
| Breast cancer type 1 susceptibility protein | BRCA1 | 16581 |
| Breast cancer type 2 susceptibility protein | BRCA2 | 16582 |
| CAMPATH-1 antigen | CD52 | 16583 |
| Cytochrome P450 2D6 | CYP2D6 | 16584 |
| Deoxycytidine kinase | dCK | 16585 |
| Dihydrofolate reductase | DHFR | 16586 |
| Dihydropyrimidine dehydrogenase [NADP(+)] | DPYD | 16587 |
| Epidermal growth factor receptor | EGFR | 16588 |
| DNA excision repair protein ERCC-1 | ERCC1 | 16589 |
| Estrogen Receptor | ESR | 16590 |
| Low affinity immunoglobulin gamma Fc region | FCGR3A | 16591 |
| receptor III-A | | |
| Receptor tyrosine-protein kinase erbB-2 | HER2 or ERBB2 | 16592 |
| Insulin-like growth factor 1 receptor | IGF1R | 16593 |
| GTPase KRas | KRAS | 16594 |
| Multidrug resistance protein 1 | MDR1 or ABCB1 | 16595 |
| Methylated-DNA--protein-cysteine methyltransferase | MGMT | 16596 |
| Multidrug resistance-associated protein 1 | MRP1 or ABCC1 | 16597 |
| Progesterone Receptor | PGR | 16598 |
| Regulator of G-protein signaling 10 | RGS10 | 16599 |
| Suppressor of cytokine signaling 3 | SOCS-3 | 16600 |
| Thymidylate synthase | TYMS | 16601 |
| UDP-glucuronosyltransferase 1-1 | UGT1A1 | 16602 |

In some embodiments, a T-cell of the disclosure is modified to silence or reduce expression of one or more gene(s) encoding a growth advantage factor to produce an armored T-cell. Silencing or reducing expression of an oncogene can confer a growth advantage for an armored T-cell of the disclosure. For example, silencing or reducing expression (e.g. disrupting expression) of a TET2 gene during a CAR-T manufacturing process results in the generation of an armored CAR-T with a significant capacity for expansion and subsequent eradication of a tumor when compared to a non-armored CAR-T lacking this capacity for expansion. This strategy may be coupled to a safety switch (e.g. an iC9 safety switch of the disclosure), which allows for the targeted disruption of an armored CAR-T-cell in the event of an adverse reaction from a subject or uncontrolled growth of the armored CAR-T. Exemplary growth advantage factors include, but are not limited to, the factors provided in Table 7.

TABLE 7
Exemplary Growth Advantage Factors.

| Full Name | Abbreviation | SEQ ID NO: |
|---|---|---|
| Ten Eleven Translocation 2 | TET2 | 16603 |
| DNA (cytosine-5)-methyltransferase 3A | DNMT3A | 16604 |
| Transforming protein RhoA | RHOA | 16605 |
| Proto-oncogene vav | VAV1 | 16606 |
| Rhombotin-2 | LMO2 | 16607 |
| T-cell acute lymphocytic leukemia | TALI | 16608 |
| protein 1 | | |
| Suppressor of cytokine signaling 1 | SOCS1 | 16609 |
| herpes virus entry mediator | HVEM | 16610 |
| T cell death-associated gene 8 | TDAG8 | 16611 |
| BCL6 corepressor | BCOR | 16612 |
| B and T cell attenuator | BTLA | 16613 |
| SPARC-like protein 1 | SPARCL1 | 16614 |
| Msh homeobox 1-like protein | MSX1 | 16615 |

### Armored T-Cells “Null or Switch Receptor” Strategy

In some embodiments, a T-cell of the disclosure is modified to express a modified/chimeric checkpoint receptor to produce an armored T-cell of the disclosure.

In some embodiments, the modified/chimeric checkpoint receptor comprises a null receptor, decoy receptor or dominant negative receptor. A null receptor, decoy receptor or dominant negative receptor of the disclosure may be modified/chimeric receptor/protein. A null receptor, decoy receptor or dominant negative receptor of the disclosure may be truncated for expression of the intracellular signaling domain. Alternatively, or in addition, a null receptor, decoy receptor or dominant negative receptor of the disclosure may be mutated within an intracellular signaling domain at one or more amino acid positions that are determinative or required for effective signaling. Truncation or mutation of null receptor, decoy receptor or dominant negative receptor of the disclosure may result in loss of the receptor's capacity to convey or transduce a checkpoint signal to the cell or within the cell.

For example, a dilution or a blockage of an immunosuppressive checkpoint signal from a PD-L1 receptor expressed on the surface of a tumor cell may be achieved by expressing a modified/chimeric PD-1 null receptor on the surface of an armored T-cell of the disclosure, which effectively competes with the endogenous (non-modified) PD-1 receptors also expressed on the surface of the armored T-cell to reduce or inhibit the transduction of the immunosuppressive checkpoint signal through endogenous PD-1 receptors of the armored T cell. In this exemplary embodiment, competition between the two different receptors for binding to PD-L1 expressed on the tumor cell reduces or diminishes a level of effective checkpoint signaling, thereby enhancing a therapeutic potential of the armored T-cell expressing the PD-1 null receptor.

In some embodiments, the modified/chimeric checkpoint receptor comprises a null receptor, decoy receptor or dominant negative receptor that is a transmembrane receptor.

In some embodiments, the modified/chimeric checkpoint receptor comprises a null receptor, decoy receptor or dominant negative receptor that is a membrane-associated or membrane-linked receptor/protein.

In some embodiments, the modified/chimeric checkpoint receptor comprises a null receptor, decoy receptor or dominant negative receptor that is an intracellular receptor/protein.

In some embodiments, the modified/chimeric checkpoint receptor comprises a null receptor, decoy receptor or dominant negative receptor that is an intracellular receptor/protein. Exemplary null, decoy, or dominant negative intracellular receptors/proteins of the disclosure include, but are not limited to, signaling components downstream of an inhibitory checkpoint signal (as provided, for example, in Tables 1 and 2), a transcription factor (as provided, for example, in Table 3), a cytokine or a cytokine receptor, a chemokine or a chemokine receptor, a cell death or apoptosis receptor/ligand (as provided, for example, in Table 4), a metabolic sensing molecule (as provided, for example, in Table 5), a protein conferring sensitivity to a cancer therapy (as provided, for example, in Table 6), and an oncogene or a tumor suppressor gene (as provided, for example, in Table 7). Exemplary cytokines, cytokine receptors, chemokines and chemokine receptors of the disclosure include, but are not limited to, the cytokines and cytokine receptors as well as chemokines and chemokine receptors provided in Table 8.

TABLE 8
Exemplary Cytokines, Cytokine receptors,
Chemokines and Chemokine Receptors.

| Full Name | Abbreviation | SEQ ID NO: |
|---|---|---|
| 4-1BB Ligand | 4-1BBL | 16616 |
| Tumor necrosis factor receptor | Apo3 or TNFRSF25 | 16617 |
| superfamily member 25 | | |
| Tumor necrosis factor receptor | APRIL or TNFRSF13 | 16618 |
| superfamily member 13 | | |
| Bcl2-associated agonist of cell death | Bcl-xL or BAD | 16619 |
| Tumor necrosis factor receptor | BCMA or TNFRSF17 | 16620 |
| superfamily member 17 | | |
| C-C motif chemokine 1 | CCL1 | 16621 |
| C-C motif chemokine 11 | CCL11 | 16622 |
| C-C motif chemokine 13 | CCL13 | 16623 |
| C-C motif chemokine 14 | CCL14 | 16624 |
| C-C motif chemokine 15 | CCL15 | 16625 |
| C-C motif chemokine 16 | CCL16 | 16626 |
| C-C motif chemokine 17 | CCL17 | 16627 |
| C-C motif chemokine 18 | CCL18 | 16628 |
| C-C motif chemokine 19 | CCL19 | 16629 |
| C-C motif chemokine 2 | CCL2 | 16630 |
| C-C motif chemokine 20 | CCL20 | 16631 |
| C-C motif chemokine 21 | CCL21 | 16632 |
| C-C motif chemokine 22 | CCL22 | 16633 |
| C-C motif chemokine 23 | CCL23 | 16634 |
| C-C motif chemokine 24 | CCL24 | 16635 |
| C-C motif chemokine 25 | CCL25 | 16636 |
| C-C motif chemokine 26 | CCL26 | 16637 |
| C-C motif chemokine 27 | CCL27 | 16638 |
| C-C motif chemokine 28 | CCL28 | 16639 |
| C-C motif chemokine 3 | CCL3 | 16640 |
| C-C motif chemokine 4 | CCL4 | 16641 |
| C-C motif chemokine 5 | CCL5 | 16642 |
| C-C motif chemokine 7 | CCL7 | 16643 |
| C-C motif chemokine 8 | CCL8 | 16644 |
| C-C chemokine receptor type 1 | CCR1 | 16645 |
| C-C chemokine receptor type 10 | CCR10 | 16646 |
| C-C chemokine receptor type 11 | CCR11 | 16647 |
| C-C chemokine receptor type 2 | CCR2 | 16648 |
| C-C chemokine receptor type 3 | CCR3 | 16649 |
| C-C chemokine receptor type 4 | CCR4 | 16650 |
| C-C chemokine receptor type 5 | CCR5 | 16651 |
| C-C chemokine receptor type 6 | CCR6 | 16652 |
| C-C chemokine receptor type 7 | CCR7 | 16653 |
| C-C chemokine receptor type 8 | CCR8 | 16654 |
| C-C chemokine receptor type 9 | CCR9 | 16655 |
| Granulocyte colony-stimulating factor | CD114 or CSF3R | 16656 |
| receptor | | |
| Macrophage colony-stimulating factor 1 | CD115 or CSFIR | 16657 |
| receptor | | |
| Granulocyte-macrophage colony- | CD116 or CSF2RA | 16658 |
| stimulating factor receptor subunit alpha | | |
| Mast/stem cell growth factor receptor | CD117 or KIT | 16659 |
| Kit | | |
| Leukemia inhibitory factor receptor | CD118 or LIFR | 16660 |
| Tumor necrosis factor receptor | CD120a or TNFRSF1A | 16661 |
| superfamily member 1A | | |
| Tumor necrosis factor receptor | CD120b or TNFRSF1B | 16662 |
| superfamily member 1B | | |
| Interleukin-1 receptor type 1 | CD121a or IL1R1 | 16663 |
| Interleukin-2 receptor subunit beta | CD122 or IL2RB | 16664 |
| Interleukin-3 receptor subunit alpha | CD123 or IL3RA | 16665 |
| Interleukin-4 receptor subunit alpha | CD124 or IL4R | 16666 |
| Interleukin-6 receptor subunit alpha | CD126 or IL6R | 16667 |
| Interleukin-7 receptor subunit alpha | CD127 or IL7R | 16668 |
| Interleukin-6 receptor subunit beta | CD130 or IL6ST | 16669 |
| Cytokine receptor common subunit | CD132 or IL2RG | 16670 |
| gamma | | |
| Tumor necrosis factor ligand | CD153 or TNFSF8 | 16671 |
| superfamily member 8 | | |
| CD40 ligand | CD154 or CD40L | 16672 |
| Tumor necrosis factor ligand | CD178 or FASLG | 16673 |
| superfamily member 6 | | |
| Interleukin-12 receptor subunit beta-1 | CD212 or IL12RB1 | 16674 |
| Interleukin-13 receptor subunit alpha-1 | CD213a1 or IL13RA1 | 16675 |
| Interleukin-13 receptor subunit alpha-2 | CD213a2 or IL13RA2 | 16676 |
| Interleukin-2 receptor subunit alpha | CD25 or IL2RA | 16677 |
| CD27 antigen | CD27 | 16678 |
| Tumor necrosis factor receptor | CD30 or TNFRSF8 | 16679 |
| superfamily member 8 | | |
| T-cell surface glycoprotein CD4 | CD4 | 16680 |
| Tumor necrosis factor receptor | CD40 or TNFRSF5 | 16681 |
| superfamily member 5 | | |
| CD70 antigen | CD70 | 16682 |
| Tumor necrosis factor receptor | CD95 or FAS or | 16683 |
| superfamily member 6 | FNFRSF6 | |
| Granulocyte-macrophage colony- | CDw116 or CSF2RA | 16684 |
| stimulating factor receptor subunit alpha | | |
| Interferon gamma receptor 1 | CDw119 or IFNGR1 | 16685 |
| Interleukin-1 receptor type 2 | CDw121b or IL1R2 | 16686 |
| Interleukin-5 receptor subunit alpha | CDw125 or IL5RA | 16687 |
| Cytokine receptor common subunit beta | CDw131 or CSF2RB | 16688 |
| Tumor necrosis factor receptor | CDw137 or TNFRSF9 | 16689 |
| superfamily member 9 | | |
| Interleukin-10 receptor | CDw210 or IL10R | 16690 |
| Interleukin-17 receptor A | CDw217 or IL17RA | 16691 |
| C-X3-C motif chemokine 1 | CX3CL1 | 16692 |
| CX3C chemokine receptor 1 | CX3CR1 | 16693 |
| C-X-C motif chemokine 1 | CXCL1 | 16694 |
| C-X-C motif chemokine 10 | CXCL10 | 16695 |
| C-X-C motif chemokine 11 | CXCL11 | 16696 |
| C-X-C motif chemokine 12 | CXCL12 | 16697 |
| C-X-C motif chemokine 13 | CXCL13 | 16698 |
| C-X-C motif chemokine 14 | CXCL14 | 16699 |
| C-X-C motif chemokine 16 | CXCL16 | 16700 |
| C-X-C motif chemokine 2 | CXCL2 | 16701 |
| C-X-C motif chemokine 3 | CXCL3 | 16702 |
| C-X-C motif chemokine 4 | CXCL4 | 16703 |
| C-X-C motif chemokine 5 | CXCL5 | 16704 |
| C-X-C motif chemokine 6 | CXCL6 | 16705 |
| C-X-C motif chemokine 7 | CXCL7 | 16706 |
| C-X-C motif chemokine 8 | CXCL8 | 16707 |
| C-X-C motif chemokine 9 | CXCL9 | 16708 |
| C-X-C chemokine receptor type 1 | CXCR1 | 16709 |
| C-X-C chemokine receptor type 2 | CXCR2 | 16710 |
| C-X-C chemokine receptor type 3 | CXCR3 | 16711 |
| C-X-C chemokine receptor type 4 | CXCR4 | 16712 |
| C-X-C chemokine receptor type 5 | CXCR5 | 16713 |
| C-X-C chemokine receptor type 6 | CXCR6 | 16714 |
| C-X-C chemokine receptor type 7 | CXCR7 | 16715 |
| Atypical chemokine receptor 1 | DARC or ACKR1 | 16716 |
| Erythropoietin | Epo | 16717 |
| Erythropoietin receptor | EpoR | 16718 |
| Receptor-type tyrosine-protein kinase | Flt-3 | 16719 |
| FLT3 | | |
| FLT3 Ligand | Flt-3L | 16720 |
| Granulocyte colony-stimulating factor | G-CSF or GSF3R | 16721 |
| receptor | | |
| Tumor necrosis factor receptor | GITR or TNFRSF18 | 16722 |
| superfamily member 18 | | |
| GITR Ligand | GITRL | 16723 |
| Cytokine receptor common subunit beta | GM-CSF or CSF2RB | 16724 |
| Interleukin-6 receptor subunit beta | gp130 or IL6ST | 16725 |
| Tumor necrosis factor receptor | HVEM or TNFRSF14 | 16726 |
| superfamily member 14 | | |
| Interferon gamma | IENγ | 16727 |
| Interferon gamma receptor 2 | IFNGR2 | 16728 |
| Interferon-alpha | IFN-α | 16729 |
| Interferon-beta | IFN-β | 16730 |
| Interleukin-1 alpha | IL1 | 16731 |
| Interleukin-10 | IL10 | 16732 |
| Interleukin-10 receptor | IL10R | 16733 |
| Interleukin-11 | IL-11 | 16734 |
| Interleukin-11 receptor alpha | IL-11Ra | 16735 |
| Interleukin-12 | IL12 | 16736 |
| Interleukin-13 | IL13 | 16737 |
| Interleukin-13 receptor | IL13R | 16738 |
| Interleukin-14 | IL-14 | 16739 |
| Interleukin-15 | IL15 | 16740 |
| Interleukin-15 receptor alpha | IL-15Ra | 16741 |
| Interleukin-16 | IL-16 | 16742 |
| Interleukin-17 | IL17 | 16743 |
| Interleukin-17 receptor | IL17R | 16744 |
| Interleukin-18 | IL18 | 16745 |
| Interleukin-1 receptor alpha | IL-1RA | 16746 |
| Interleukin-1 alpha | IL-1α | 16747 |
| Interleukin-1beta | IL-1β | 16748 |
| Interleukin-2 | IL2 | 16749 |
| Interleukin-20 | IL-20 | 16750 |
| Interleukin-20 receptor alpha | IL-20Rα | 16751 |
| Interleukin-20 receptor beta | IL-20Rβ | 16752 |
| Interleukin-21 | IL21 | 16753 |
| Interleukin-3 | IL-3 | 16754 |
| Interleukin-35 | IL35 | 16755 |
| Interleukin-4 | IL4 | 16756 |
| Interleukin-4 receptor | IL4R | 16757 |
| Interleukin-5 | IL5 | 16758 |
| Interleukin-5 receptor | IL5R | 16759 |
| Interleukin-6 | IL6 | 16760 |
| Interleukin-6 receptor | IL6R | 16761 |
| Interleukin-7 | IL7 | 16762 |
| Interleukin-9 receptor | IL-9R | 16763 |
| Leukemia inhibitory factor | LIF | 16764 |
| Leukemia inhibitory factor receptor | LIFR | 16765 |
| tumor necrosis factor superfamily | LIGHT or TNFSF14 | 16766 |
| member 14 | | |
| Tumor necrosis factor receptor | LTβR or TNFRSF3 | 16767 |
| superfamily member 3 | | |
| Lymphotoxin-beta | LT-β | 16768 |
| Macrophage colony-stimulating factor 1 | M-CSF | 16769 |
| Tumor necrosis factor receptor | OPG or TNFRSF11B | 16770 |
| superfamily member 11B | | |
| Oncostatin-M | OSM | 16771 |
| Oncostatin-M receptor | OSMR | 16772 |
| Tumor necrosis factor receptor | OX40 or TNFRSF4 | 16773 |
| superfamily member 4 | | |
| Tumor necrosis factor ligand | OX40L or TNFSF4 | 16774 |
| superfamily member 4 | | |
| Tumor necrosis factor receptor | RANK or TNFRSF11A | 16775 |
| superfamily member 11A | | |
| Kit Ligand | SCF or KITLG | 16776 |
| Tumor necrosis factor receptor | TACI or TNFRSF13B | 16777 |
| superfamily member 13B | | |
| Tumor necrosis factor ligand | TALL-1 or TNFSF13B | 16778 |
| superfamily member 13B | | |
| TGF-beta receptor type-1 | TGF-βR1 | 16779 |
| TGF-beta receptor type-2 | TGF-βR2 | 16780 |
| TGF-beta receptor type-3 | TGF-βR3 | 16781 |
| Transforming growth factor beta-1 | TGF-β1 | 16782 |
| Transforming growth factor beta-2 | TGF-β2 | 16783 |
| Transforming growth factor beta-3 | TGF-β3 | 16784 |
| Tumor necrosis factor alpha | TNF or TNF-α | 16785 |
| Tumor necrosis factor beta | TNF-β | 16786 |
| Thyroid peroxidase | Tpo | 16787 |
| Thyroid peroxidase receptor | TpoR | 16788 |
| Tumor necrosis factor ligand | TRAIL or TNFSF10 | 16789 |
| superfamily member 10 | | |
| Tumor necrosis factor receptor | TRAILR1 or | 16790 |
| superfamily member 10A | TNFRSF10A | |
| Tumor necrosis factor receptor | TRAILR2 or | 16791 |
| superfamily member 10B | TNFRSF10B | |
| Tumor necrosis factor ligand | TRANCE or TNFSF11 | 16792 |
| superfamily member 11 | | |
| Tumor necrosis factor ligand | TWEAK or TNFSF11 | 16793 |
| superfamily member 12 | | |
| Lymphotactin | XCL1 | 16794 |
| Cytokine SCM-1 beta | XCL2 | 16795 |

In some embodiments, the modified/chimeric checkpoint receptor comprises a switch receptor. Exemplary switch receptors may comprise a modified/chimeric receptor/protein of the disclosure wherein a native or wild type intracellular signaling domain is switched or replaced with a different intracellular signaling domain that is either non-native to the protein and/or not a wild-type domain. For example, replacement of an inhibitory signaling domain with a stimulatory signaling domain would switch an immunosuppressive signal into an immunostimulatory signal. Alternatively, replacement of an inhibitory signaling domain with a different inhibitory domain can reduce or enhance the level of inhibitory signaling. Expression or overexpression, of a switch receptor can result in the dilution and/or blockage of a cognate checkpoint signal via competition with an endogenous wildtype checkpoint receptor (not a switch receptor) for binding to the cognate checkpoint receptor expressed within the immunosuppressive tumor microenvironment. Armored T cells of the disclosure may comprise a sequence encoding switch receptors of the disclosure, leading to the expression of one or more switch receptors of the disclosure, and consequently, altering an activity of an armored T-cell of the disclosure. Armored T cells of the disclosure may express a switch receptor of the disclosure that targets an intracellularly expressed protein downstream of a checkpoint receptor, a transcription factor, a cytokine receptor, a death receptor, a metabolic sensing molecule, a cancer therapy, an oncogene, and/or a tumor suppressor protein or gene of the disclosure.

Exemplary switch receptors of the disclosure may comprise or may be derived from a protein including, but are not limited to, the signaling components downstream of an inhibitory checkpoint signal (as provided, for example, in Tables 1 and 2), a transcription factor (as provided, for example, in Table 3), a cytokine or a cytokine receptor, a chemokine or a chemokine receptor, a cell death or apoptosis receptor/ligand (as provided, for example, in Table 4), a metabolic sensing molecule (as provided, for example, in Table 5), a protein conferring sensitivity to a cancer therapy (as provided, for example, in Table 6), and an oncogene or a tumor suppressor gene (as provided, for example, in Table 7). Exemplary cytokines, cytokine receptors, chemokines and chemokine receptors of the disclosure include, but are not limited to, the cytokines and cytokine receptors as well as chemokines and chemokine receptors provided in Table 8.

### Armored T-Cells “Synthetic Gene Expression” Strategy

In some embodiments, a T-cell of the disclosure is modified to express chimeric ligand receptor (CLR) or a chimeric antigen receptor (CAR) that mediates conditional gene expression to produce an armored T-cell of the disclosure. The combination of the CLR/CAR and the condition gene expression system in the nucleus of the armored T cell constitutes a synthetic gene expression system that is conditionally activated upon binding of cognate ligand(s) with CLR or cognate antigen(s) with CAR. This system may help to ‘armor’ or enhance therapeutic potential of modified T cells by reducing or limiting synthetic gene expression at the site of ligand or antigen binding, at or within the tumor environment for example.

### Exogenous Receptors

In some embodiments, the armored T-cell comprises a composition comprising (a) an inducible transgene construct, comprising a sequence encoding an inducible promoter and a sequence encoding a transgene, and (b) a receptor construct, comprising a sequence encoding a constitutive promoter and a sequence encoding an exogenous receptor, such as a CLR or CAR, wherein, upon integration of the construct of (a) and the construct of (b) into a genomic sequence of a cell, the exogenous receptor is expressed, and wherein the exogenous receptor, upon binding a ligand or antigen, transduces an intracellular signal that targets directly or indirectly the inducible promoter regulating expression of the inducible transgene (a) to modify gene expression.

In some embodiments of a synthetic gene expression system of the disclosure, the composition modifies gene expression by decreasing gene expression. In some embodiments, the composition modifies gene expression by transiently modifying gene expression (e.g. for the duration of binding of the ligand to the exogenous receptor). In some embodiments, the composition modifies gene expression acutely (e.g. the ligand reversibly binds to the exogenous receptor). In some embodiments, the composition modifies gene expression chronically (e.g. the ligand irreversibly binds to the exogenous receptor).

In some embodiments of the compositions of the disclosure, the exogenous receptor of (b) comprises an endogenous receptor with respect to the genomic sequence of the cell. Exemplary receptors include, but are not limited to, intracellular receptors, cell-surface receptors, transmembrane receptors, ligand-gated ion channels, and G-protein coupled receptors.

In some embodiments of the compositions of the disclosure, the exogenous receptor of (b) comprises a non-naturally occurring receptor. In some embodiments, the non-naturally occurring receptor is a synthetic, modified, recombinant, mutant or chimeric receptor. In some embodiments, the non-naturally occurring receptor comprises one or more sequences isolated or derived from a T-cell receptor (TCR). In some embodiments, the non-naturally occurring receptor comprises one or more sequences isolated or derived from a scaffold protein. In some embodiments, including those wherein the non-naturally occurring receptor does not comprise a transmembrane domain, the non-naturally occurring receptor interacts with a second transmembrane, membrane-bound and/or an intracellular receptor that, following contact with the non-naturally occurring receptor, transduces an intracellular signal.

In some embodiments of the compositions of the disclosure, the exogenous receptor of (b) comprises a non-naturally occurring receptor. In some embodiments, the non-naturally occurring receptor is a synthetic, modified, recombinant, mutant or chimeric receptor. In some embodiments, the non-naturally occurring receptor comprises one or more sequences isolated or derived from a T-cell receptor (TCR). In some embodiments, the non-naturally occurring receptor comprises one or more sequences isolated or derived from a scaffold protein. In some embodiments, the non-naturally occurring receptor comprises a transmembrane domain. In some embodiments, the non-naturally occurring receptor interacts with an intracellular receptor that transduces an intracellular signal. In some embodiments, the non-naturally occurring receptor comprises an intracellular signalling domain. In some embodiments, the non-naturally occurring receptor is a chimeric ligand receptor (CLR). In some embodiments, the CLR is a chimeric antigen receptor (CAR).

In some embodiments of the compositions of the disclosure, the exogenous receptor of (b) comprises a non-naturally occurring receptor. In some embodiments, the CLR is a chimeric antigen receptor (CAR). In some embodiments, the chimeric ligand receptor comprises (a) an ectodomain comprising a ligand recognition region, wherein the ligand recognition region comprises at least scaffold protein; (b) a transmembrane domain, and (c) an endodomain comprising at least one costimulatory domain. In some embodiments, the ectodomain of (a) further comprises a signal peptide. In some embodiments, the ectodomain of (a) further comprises a hinge between the ligand recognition region and the transmembrane domain.

In some embodiments of the CLR/CARs of the disclosure, the signal peptide comprises a sequence encoding a human CD2, CD3δ, CD3ε, CD3γ, CD3ζ, CD4, CD8α, CD19, CD28, 4-1BB or GM-CSFR signal peptide. In some embodiments, the signal peptide comprises a sequence encoding a human CD8α signal peptide. In some embodiments, the signal peptide comprises an amino acid sequence comprising MALPVTALLLPLALLLHAARP (SEQ ID NO: 18004). In some embodiments, the signal peptide is encoded by a nucleic acid sequence comprising atggcactgccagtcaccgccctgctgctgcctctggctctgctgctgcacgcagctagacca.

In some embodiments of the CLR/CARs of the disclosure, the transmembrane domain comprises a sequence encoding a human CD2, CD3δ, CD3ε, CD3γ, CD3ζ, CD4, CD8α, CD19, CD28, 4-1BB or GM-CSFR transmembrane domain. In some embodiments, the transmembrane domain comprises a sequence encoding a human CD8α transmembrane domain. In some embodiments, the transmembrane domain comprises an amino acid sequence comprising IYIWAPLAGTCGVLLLSLVITLYC (SEQ ID NO: 18006). In some embodiments, the transmembrane domain is encoded by a nucleic acid sequence comprising

(SEQ ID NO: 18007)
atctacatttgggcaccactggccgggacctgtggagtgctgctgctgag
cctggtcatcacactgtactgc.

In some embodiments of the CLR/CARs of the disclosure, the endodomain comprises a human CD3ζ endodomain. In some embodiments, the at least one costimulatory domain comprises a human 4-1BB, CD28, CD40, ICOS, MyD88, OX-40 intracellular segment, or any combination thereof. In some embodiments, the at least one costimulatory domain comprises a human CD28 and/or a 4-1BB costimulatory domain. In some embodiments, the CD3ζ costimulatory domain comprises an amino acid sequence comprising

(SEQ ID NO: 18008)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALP
PR.

In some embodiments, the CD3ζ costimulatory domain is encoded by a nucleic acid sequence comprising

(SEQ ID NO: 18010)
cgcgtgaagtttagtcgatcagcagatgccccagcttacaaacagggaca
gaaccagctgtataacgagctgaatctgggccgccgagaggaatatgacg
tgctggataagcggagaggacgcgaccccgaaatgggaggcaagcccagg
cgcaaaaaccctcaggaaggcctgtataacgagctgcagaaggacaaaat
ggcagaagcctattctgagatcggcatgaagggggagcgacggagaggca
aagggcacgatgggctgtaccagggactgagcaccgccacaaaggacacc
tatgatgctctgcatatgcaggcactgcctccaagg.

In some embodiments, the 4-1BB costimulatory domain comprises an amino acid sequence comprising

(SEQ ID NO: 18011)
KRGRKKLLYIFKQPFMRPVQTTQEEDGCSCRFPEEEEGGCEL.

In some embodiments, the 4-1BB costimulatory domain is encoded by a nucleic acid sequence comprising

(SEQ ID NO: 18013)
aagagaggcaggaagaaactgctgtatattttcaaacagcccttcatgcg
ccccgtgcagactacccaggaggaagacgggtgctcctgtcgattccctg
aggaagaggaaggcgggtgtgagctg.

In some embodiments, the 4-1BB costimulatory domain is located between the transmembrane domain and the CD28 costimulatory domain.

In some embodiments of the CLR/CARs of the disclosure, the hinge comprises a sequence derived from a human CD8α, IgG4, and/or CD4 sequence. In some embodiments, the hinge comprises a sequence derived from a human CD8α sequence. In some embodiments, the hinge comprises an amino acid sequence comprising

(SEQ ID NO: 18014)
TTTPAPRPPTPAPTIASQPLSLRPEACRPAAGGAVHTRGLDFACD.

In some embodiments, the hinge is encoded by a nucleic acid sequence comprising

(SEQ ID NO: 18016)
ACCACAACCCCTGCCCCCAGACCTCCCACACCCGCCCCTACCATCGCGAG
TCAGCCCCTGAGTCTGAGACCTGAGGCCTGCAGGCCAGCTGCAGGAGGAG
CTGTGCACACCAGGGGCCTGGACTTCGCCTGCGAC
or
(SEQ ID NO: 18017)
ACCACAACCCCTGCCCCCAGACCTCCCACACCCGCCCCTACCATCGCGAG
TCAGCCCCTGAGTCTGAGACCTGAGGCCTGCAGGCCAGCTGCAGGAGGAG
CTGTGCACACCAGGGGCCTGGACTTCGCCTGCGAC.

In some embodiments, the at least one protein scaffold specifically binds the ligand.

In some embodiments of the compositions of the disclosure, the exogenous receptor of (b) comprises a non-naturally occurring receptor. In some embodiments, the CLR is a chimeric antigen receptor (CAR). In some embodiments, the chimeric ligand receptor comprises (a) an ectodomain comprising a ligand recognition region, wherein the ligand recognition region comprises at least scaffold protein; (b) a transmembrane domain, and (c) an endodomain comprising at least one costimulatory domain. In some embodiments, the at least one protein scaffold comprises an antibody, an antibody fragment, a single domain antibody, a single chain antibody, an antibody mimetic, or a Centyrin (referred to herein as a CARTyrin). In some embodiments, the ligand recognition region comprises one or more of an antibody, an antibody fragment, a single domain antibody, a single chain antibody, an antibody mimetic, and a Centyrin. In some embodiments, the single domain antibody comprises or consists of a VHH or a VH (referred to herein as a VCAR). In some embodiments, the single domain antibody comprises or consists of a VHH or a VH comprising human complementarity determining regions (CDRs). In some embodiments, the VH is a recombinant or chimeric protein. In some embodiments, the VH is a recombinant or chimeric human protein. In some embodiments, the antibody mimetic comprises or consists of an affibody, an afflilin, an affimer, an affitin, an alphabody, an anticalin, an avimer, a DARPin, a Fynomer, a Kunitz domain peptide or a monobody. In some embodiments, the Centyrin comprises or consists of a consensus sequence of at least one fibronectin type III (FN3) domain.

In some embodiments of the compositions of the disclosure, the exogenous receptor of (b) comprises a non-naturally occurring receptor. In some embodiments, the CLR is a chimeric antigen receptor (CAR). In some embodiments, the chimeric ligand receptor comprises (a) an ectodomain comprising a ligand recognition region, wherein the ligand recognition region comprises at least scaffold protein; (b) a transmembrane domain, and (c) an endodomain comprising at least one costimulatory domain. In some embodiments, the Centyrin comprises or consists of a consensus sequence of at least one fibronectin type III (FN3) domain. In some embodiments, the at least one fibronectin type III (FN3) domain is derived from a human protein. In some embodiments, the human protein is Tenascin-C. In some embodiments, the consensus sequence comprises

(SEQ ID NO: 18018)
LPAPKNLVVSEVTEDSLRLSWTAPDAAFDSFLIQYQESEKVGEAINLTVP
GSERSYDLTGLKPGTEYTVSIYGVKGGHRSNPLSAEFTT.

In some embodiments, the consensus sequence comprises

(SEQ ID NO: 18019)
MLPAPKNLVVSEVTEDSLRLSWTAPDAAFDSFLIQYQESEKVGEAI
NLTVPGSERSYDLTGLKPGTEYTVSIYGVKGGHRSNPLSAEFTT.

In some embodiments, the consensus sequence is modified at one or more positions within (a) a A-B loop comprising or consisting of the amino acid residues TEDS (SEQ ID NO:18020) at positions 13-16 of the consensus sequence; (b) a B-C loop comprising or consisting of the amino acid residues TAPDAAF (SEQ ID NO:18021) at positions 22-28 of the consensus sequence; (c) a C-D loop comprising or consisting of the amino acid residues SEKVGE (SEQ ID NO:18022) at positions 38-43 of the consensus sequence; (d) a D-E loop comprising or consisting of the amino acid residues GSER (SEQ ID NO:18023) at positions 51-54 of the consensus sequence; (e) a E-F loop comprising or consisting of the amino acid residues GLKPG (SEQ ID NO:18024) at positions 60-64 of the consensus sequence; (0 a F-G loop comprising or consisting of the amino acid residues KGGHRSN (SEQ ID NO:18025) at positions 75-81 of the consensus sequence; or (g) any combination of (a)-(f). In some embodiments, the Centyrin comprises a consensus sequence of at least 5 fibronectin type III (FN3) domains. In some embodiments, the Centyrin comprises a consensus sequence of at least 10 fibronectin type III (FN3) domains. In some embodiments, the Centyrin comprises a consensus sequence of at least 15 fibronectin type III (FN3) domains. In some embodiments, the scaffold binds an antigen with at least one affinity selected from a K_{D} of less than or equal to 10⁻⁹ M, less than or equal to 10⁻¹⁰ M, less than or equal to 10⁻¹¹ M, less than or equal to 10⁻¹² M, less than or equal to 10⁻¹³ M, less than or equal to 10⁻¹⁴ M, and less than or equal to 10⁻¹⁵ M. In some embodiments, the K_{D} is determined by surface plasmon resonance.

### Inducible Promoters

In some embodiments of the compositions of the disclosure, the sequence encoding the inducible promoter of (a) comprises a sequence encoding an NFκB promoter. In some embodiments of the compositions of the disclosure, the sequence encoding the inducible promoter of (a) comprises a sequence encoding an interferon (IFN) promoter or a sequence encoding an interleukin-2 promoter. In some embodiments, the interferon (IFN) promoter is an IFNγ promoter. In some embodiments of the compositions of the disclosure, the inducible promoter is isolated or derived from the promoter of a cytokine or a chemokine. In some embodiments, the cytokine or chemokine comprises IL2, IL3, IL4, IL5, IL6, IL10, IL12, IL13, IL17A/F, IL21, IL22, IL23, transforming growth factor beta (TGFβ), colony stimulating factor 2 (GM-CSF), interferon gamma (IFNγ), Tumor necrosis factor (TNFα), LTα, perforin, Granzyme C (Gzmc), Granzyme B (Gzmb), C-C motif chemokine ligand 5 (CCLS), C-C motif chemokine ligand 4 (Ccl4), C-C motif chemokine ligand 3 (Ccl3), X-C motif chemokine ligand 1 (Xcl1) and LIF interleukin 6 family cytokine (Lif).

In some embodiments of the compositions of the disclosure, the inducible promoter is isolated or derived from the promoter of a gene comprising a surface protein involved in cell differentiation, activation, exhaustion and function. In some embodiments, the gene comprises CD69, CD71, CTLA4, PD-1, TIGIT, LAG3, TIM-3, GITR, MHCII, COX-2, FASL and 4-1BB.

In some embodiments of the compositions of the disclosure, the inducible promoter is isolated or derived from the promoter of a gene involved in CD metabolism and differentiation. In some embodiments of the compositions of the disclosure, the inducible promoter is isolated or derived from the promoter of Nr4a1, Nr4a3, Tnfrsf9 (4-1BB), Sema7a, Zfp3612, Gadd45b, Dusp5, Dusp6 and Neto2.

### Inducible Transgene

In some embodiments, the inducible transgene construct comprises or drives expression of a signaling component downstream of an inhibitory checkpoint signal (as provided, for example, in Tables 1 and 2), a transcription factor (as provided, for example, in Table 3), a cytokine or a cytokine receptor, a chemokine or a chemokine receptor, a cell death or apoptosis receptor/ligand (as provided, for example, in Table 4), a metabolic sensing molecule (as provided, for example, in Table 5), a protein conferring sensitivity to a cancer therapy (as provided, for example, in Table 6 and/or 9), and an oncogene or a tumor suppressor gene (as provided, for example, in Table 7). Exemplary cytokines, cytokine receptors, chemokines and chemokine receptors of the disclosure include, but are not limited to, the cytokines and cytokine receptors as well as chemokines and chemokine receptors Provided in Table 8.

TABLE 9
Exemplary therapeutic proteins (and proteins to enhance CAR-T efficacy).

| Gene Name | Gene Description | Protein SEQ ID NO |
|---|---|---|
| A1BG | Alpha-1-B glycoprotein | SEQ ID NOS: 1-2 |
| A2M | Alpha-2-macroglobulin | SEQ ID NOS: 3-6 |
| A2ML1 | Alpha-2-macroglobulin-like 1 | SEQ ID NOS: 7-12 |
| A4GNT | Alpha-1,4-N-acetylglucosaminyltransferase | SEQ ID NO: 13 |
| AADACL2 | Arylacetamide deacetylase-like 2 | SEQ ID NOS: 14-15 |
| AANAT | Aralkylamine N-acetyltransferase | SEQ ID NOS: 16-19 |
| ABCG1 | ATP-binding cassette, sub-family G | SEQ ID NOS: 20-26 |
| | (WHITE), member 1 | |
| ABHD1 | Abhydrolase domain containing 1 | SEQ ID NOS: 27-31 |
| ABHD10 | Abhydrolase domain containing 10 | SEQ ID NOS: 32-35 |
| ABHD14A | Abhydrolase domain containing 14A | SEQ ID NOS: 36-40 |
| ABHD15 | Abhydrolase domain containing 15 | SEQ ID NO: 41 |
| ABI3BP | ABI family, member 3 (NESH) binding | SEQ ID NOS: 42-63 |
| | protein | |
| AC008641.1 | | SEQ ID NO: 73 |
| AC009133.22 | | SEQ ID NO: 76 |
| AC009491.2 | | SEQ ID NO: 77 |
| AC011513.3 | | SEQ ID NOS: 92-93 |
| AC136352.5 | | SEQ ID NO: 88 |
| AC145212.4 | MaFF-interacting protein | SEQ ID NO: 90 |
| AC233755.1 | | SEQ ID NO: 91 |
| ACACB | Acetyl-CoA carboxylase beta | SEQ ID NOS: 94-100 |
| ACAN | Aggrecan | SEQ ID NOS: 101-108 |
| ACE | Angiotensin I converting enzyme | SEQ ID NOS: 109-121 |
| ACHE | Acetylcholinesterase (Yt blood group) | SEQ ID NOS: 122-134 |
| ACP2 | Acid phosphatase 2, lysosomal | SEQ ID NOS: 135-142 |
| ACP5 | Acid phosphatase 5, tartrate resistant | SEQ ID NOS: 143-151 |
| ACP6 | Acid phosphatase 6, lysophosphatidic | SEQ ID NOS: 152-158 |
| ACPP | Acid phosphatase, prostate | SEQ ID NOS: 163-167 |
| ACR | Acrosin | SEQ ID NOS: 168-169 |
| ACRBP | Acrosin binding protein | SEQ ID NOS: 170-174 |
| ACRV1 | Acrosomal vesicle protein 1 | SEQ ID NOS: 175-178 |
| ACSF2 | Acyl-CoA synthetase family member 2 | SEQ ID NOS: 179-187 |
| ACTL10 | Actin-like 10 | SEQ ID NO: 188 |
| ACVR1 | Activin A receptor, type I | SEQ ID NOS: 189-197 |
| ACVR1C | Activin A receptor, type IC | SEQ ID NOS: 198-201 |
| ACVRL1 | Activin A receptor type II-like 1 | SEQ ID NOS: 202-207 |
| ACYP1 | Acylphosphatase 1, erythrocyte (common) | SEQ ID NOS: 208-213 |
| | type | |
| ACYP2 | Acylphosphatase 2, muscle type | SEQ ID NOS: 214-221 |
| ADAM10 | ADAM metallopeptidase domain 10 | SEQ ID NOS: 230-237 |
| ADAM12 | ADAM metallopeptidase domain 12 | SEQ ID NOS: 238-240 |
| ADAM15 | ADAM metallopeptidase domain 15 | SEQ ID NOS: 241-252 |
| ADAM17 | ADAM metallopeptidase domain 17 | SEQ ID NOS: 253-255 |
| ADAM18 | ADAM metallopeptidase domain 18 | SEQ ID NOS: 256-260 |
| ADAM22 | ADAM metallopeptidase domain 22 | SEQ ID NOS: 261-269 |
| ADAM28 | ADAM metallopeptidase domain 28 | SEQ ID NOS: 270-275 |
| ADAM29 | ADAM metallopeptidase domain 29 | SEQ ID NOS: 276-284 |
| ADAM32 | ADAM metallopeptidase domain 32 | SEQ ID NOS: 285-291 |
| ADAM33 | ADAM metallopeptidase domain 33 | SEQ ID NOS: 292-296 |
| ADAM7 | ADAM metallopeptidase domain 7 | SEQ ID NOS: 297-300 |
| ADAM8 | ADAM metallopeptidase domain 8 | SEQ ID NOS: 301-305 |
| ADAM9 | ADAM metallopeptidase domain 9 | SEQ ID NOS: 306-311 |
| ADAMDEC1 | AD AM-like, decysin 1 | SEQ ID NOS: 312-314 |
| ADAMTS1 | ADAM metallopeptidase with | SEQ ID NOS: 315-318 |
| | thrombospondin type 1 motif, 1 | |
| ADAMTS10 | ADAM metallopeptidase with | SEQ ID NOS: 319-324 |
| | thrombospondin type 1 motif, 10 | |
| ADAMTS12 | ADAM metallopeptidase with | SEQ ID NOS: 325-327 |
| | thrombospondin type 1 motif, 12 | |
| ADAMTS13 | ADAM metallopeptidase with | SEQ ID NOS: 328-335 |
| | thrombospondin type 1 motif, 13 | |
| ADAMTS14 | ADAM metallopeptidase with | SEQ ID NOS: 336-337 |
| | thrombospondin type 1 motif, 14 | |
| ADAMTS15 | ADAM metallopeptidase with | SEQ ID NO: 338 |
| | thrombospondin type 1 motif, 15 | |
| ADAMTS16 | ADAM metallopeptidase with | SEQ ID NOS: 339-340 |
| | thrombospondin type 1 motif, 16 | |
| ADAMTS17 | ADAM metallopeptidase with | SEQ ID NOS: 341-344 |
| | thrombospondin type 1 motif, 17 | |
| ADAMTS18 | ADAM metallopeptidase with | SEQ ID NOS: 345-348 |
| | thrombospondin type 1 motif, 18 | |
| ADAMTS19 | ADAM metallopeptidase with | SEQ ID NOS: 349-352 |
| | thrombospondin type 1 motif, 19 | |
| ADAMTS2 | ADAM metallopeptidase with | SEQ ID NOS: 353-355 |
| | thrombospondin type 1 motif, 2 | |
| ADAMTS20 | ADAM metallopeptidase with | SEQ ID NOS: 356-359 |
| | thrombospondin type 1 motif, 20 | |
| ADAMTS3 | ADAM metallopeptidase with | SEQ ID NOS: 360-361 |
| | thrombospondin type 1 motif, 3 | |
| ADAMTS5 | ADAM metallopeptidase with | SEQ ID NO: 362 |
| | thrombospondin type 1 motif, 5 | |
| ADAMTS6 | ADAM metallopeptidase with | SEQ ID NOS: 363-364 |
| | thrombospondin type 1 motif, 6 | |
| ADAMTS7 | ADAM metallopeptidase with | SEQ ID NO: 365 |
| | thrombospondin type 1 motif, 7 | |
| ADAMTS8 | ADAM metallopeptidase with | SEQ ID NO: 366 |
| | thrombospondin type 1 motif, 8 | |
| ADAMTS9 | ADAM metallopeptidase with | SEQ ID NOS: 367-371 |
| | thrombospondin type 1 motif, 9 | |
| ADAMTSL1 | ADAMTS-like 1 | SEQ ID NOS: 372-382 |
| ADAMTSL2 | ADAMTS-like 2 | SEQ ID NOS: 383-385 |
| ADAMTSL3 | ADAMTS-like 3 | SEQ ID NOS: 386-387 |
| ADAMTSL4 | ADAMTS-like 4 | SEQ ID NOS: 388-391 |
| ADAMTSL5 | ADAMTS-like 5 | SEQ ID NOS: 392-397 |
| ADCK1 | AarF domain containing kinase 1 | SEQ ID NOS: 398-402 |
| ADCYAP1 | Adenylate cyclase activating polypeptide 1 | SEQ ID NOS: 403-404 |
| | (pituitary) | |
| ADCYAP1R1 | Adenylate cyclase activating polypeptide 1 | SEQ ID NOS: 405-411 |
| | (pituitary) receptor type I | |
| ADGRA3 | Adhesion G protein-coupled receptor A3 | SEQ ID NOS: 412-416 |
| ADGRB2 | Adhesion G protein-coupled receptor B2 | SEQ ID NOS: 417-425 |
| ADGRD1 | Adhesion G protein-coupled receptor D1 | SEQ ID NOS: 426-431 |
| ADGRE3 | Adhesion G protein-coupled receptor E3 | SEQ ID NOS: 432-436 |
| ADGRE5 | Adhesion G protein-coupled receptor E5 | SEQ ID NOS: 437-442 |
| ADGRF1 | Adhesion G protein-coupled receptor F1 | SEQ ID NOS: 443-447 |
| ADGRG1 | Adhesion G protein-coupled receptor G1 | SEQ ID NOS: 448-512 |
| ADGRG5 | Adhesion G protein-coupled receptor G5 | SEQ ID NOS: 513-515 |
| ADGRG6 | Adhesion G protein-coupled receptor G6 | SEQ ID NOS: 516-523 |
| ADGRV1 | Adhesion G protein-coupled receptor V1 | SEQ ID NOS: 524-540 |
| ADI1 | Acireductone dioxygenase 1 | SEQ ID NOS: 541-543 |
| ADIG | Adipogenin | SEQ ID NOS: 544-547 |
| ADIPOQ | Adiponectin, C1Q and collagen domain | SEQ ID NOS: 548-549 |
| | containing | |
| ADM | Adrenomedullin | SEQ ID NOS: 550-557 |
| ADM2 | Adrenomedullin 2 | SEQ ID NOS: 558-559 |
| ADM5 | Adrenomedullin 5 (putative) | SEQ ID NO: 560 |
| ADPGK | ADP-dependent glucokinase | SEQ ID NOS: 561-570 |
| ADPRHL2 | ADP-ribosylhydrolase like 2 | SEQ ID NO: 571 |
| AEBP1 | AE binding protein 1 | SEQ ID NOS: 572-579 |
| AFM | Afamin | SEQ ID NO: 584 |
| AFP | Alpha-fetoprotein | SEQ ID NOS: 585-586 |
| AGA | Aspartylglucosaminidase | SEQ ID NOS: 587-589 |
| AGER | Advanced glycosylation end product- | SEQ ID NOS: 590-600 |
| | specific receptor | |
| AGK | Acylglycerol kinase | SEQ ID NOS: 601-606 |
| AGPS | Alkylglycerone phosphate synthase | SEQ ID NOS: 607-610 |
| AGR2 | Anterior gradient 2, protein disulphide | SEQ ID NOS: 611-614 |
| | isomerase family member | |
| AGR3 | Anterior gradient 3, protein disulphide | SEQ ID NOS: 615-617 |
| | isomerase family member | |
| AGRN | Agrin | SEQ ID NOS: 618-621 |
| AGRP | Agouti related neuropeptide | SEQ ID NO: 622 |
| AGT | Angiotensinogen (serpin peptidase inhibitor, | SEQ ID NO: 623 |
| | clade A, member 8) | |
| AGTPBP1 | ATP/GTP binding protein 1 | SEQ ID NOS: 624-627 |
| AGTRAP | Angiotensin II receptor-associated protein | SEQ ID NOS: 628-635 |
| AHCYL2 | Adenosylhomocysteinase-like 2 | SEQ ID NOS: 636-642 |
| AHSG | Alpha-2-HS-glycoprotein | SEQ ID NOS: 643-644 |
| AIG1 | Androgen-induced 1 | SEQ ID NOS: 645-653 |
| AK4 | Adenylate kinase 4 | SEQ ID NOS: 654-657 |
| AKAP10 | A kinase (PRKA) anchor protein 10 | SEQ ID NOS: 658-666 |
| AKR1C1 | Aldo-keto reductase family 1, member C1 | SEQ ID NOS: 667-669 |
| AL356289.1 | | SEQ ID NO: 677 |
| AL589743.1 | | SEQ ID NO: 678 |
| ALAS2 | 5′-aminolevulinate synthase 2 | SEQ ID NOS: 684-691 |
| ALB | Albumin | SEQ ID NOS: 692-701 |
| ALDH9A1 | Aldehyde dehydrogenase 9 family, member | SEQ ID NO: 702 |
| | A1 | |
| ALDOA | Aldolase A, fructose-bisphosphate | SEQ ID NOS: 703-717 |
| ALG1 | ALG1, chitobiosyldiphosphodolichol beta- | SEQ ID NOS: 718-723 |
| | mannosyltransferase | |
| ALG5 | ALG5, dolichyl-phosphate beta- | SEQ ID NOS: 724-725 |
| | glucosyltransferase | |
| ALG9 | ALG9, alpha-1,2-mannosyltransferase | SEQ ID NOS: 726-736 |
| ALKBH1 | AlkB homolog 1, histone H2A dioxygenase | SEQ ID NOS: 746-748 |
| ALKBH5 | AlkB homolog 5, RNA demethylase | SEQ ID NOS: 749-750 |
| ALPI | Alkaline phosphatase, intestinal | SEQ ID NOS: 751-752 |
| ALPL | Alkaline phosphatase, liver/bone/kidney | SEQ ID NOS: 753-757 |
| ALPP | Alkaline phosphatase, placental | SEQ ID NO: 758 |
| ALPPL2 | Alkaline phosphatase, placental-like 2 | SEQ ID NO: 759 |
| AMBN | Ameloblastin (enamel matrix protein) | SEQ ID NOS: 760-762 |
| AMBP | Alpha-1-microglobulin/bikunin precursor | SEQ ID NOS: 763-765 |
| AMELX | Amelogenin, X-linked | SEQ ID NOS: 766-768 |
| AMELY | Amelogenin, Y-linked | SEQ ID NOS: 769-770 |
| AMH | Anti-Mullerian hormone | SEQ ID NO: 771 |
| AMICA1 | Adhesion molecule, interacts with CXADR | SEQ ID NOS: 7348- |
| | antigen 1 | 7356 |
| AMPD1 | Adenosine monophosphate deaminase 1 | SEQ ID NOS: 772-774 |
| AMTN | Amelotin | SEQ ID NOS: 775-776 |
| AMY1A | Amylase, alpha 1A (salivary) | SEQ ID NOS: 777-779 |
| AMY1B | Amylase, alpha 1B (salivary) | SEQ ID NOS: 780-783 |
| AMY1C | Amylase, alpha 1C (salivary) | SEQ ID NO: 784 |
| AMY2A | Amylase, alpha 2A (pancreatic) | SEQ ID NOS: 785-787 |
| AMY2B | Amylase, alpha 2B (pancreatic) | SEQ ID NOS: 788-792 |
| ANG | Angiogenin, ribonuclease, RNase A family, | SEQ ID NOS: 793-794 |
| | 5 | |
| ANGEL1 | Angel homolog 1 (Drosophila) | SEQ ID NOS: 795-798 |
| ANGPT1 | Angiopoietin 1 | SEQ ID NOS: 799-803 |
| ANGPT2 | Angiopoietin 2 | SEQ ID NOS: 804-807 |
| ANGPT4 | Angiopoietin 4 | SEQ ID NO: 808 |
| ANGPTL1 | Angiopoietin-like 1 | SEQ ID NOS: 809-811 |
| ANGPTL2 | Angiopoietin-like 2 | SEQ ID NOS: 812-813 |
| ANGPTL3 | Angiopoietin-like 3 | SEQ ID NO: 814 |
| ANGPTL4 | Angiopoietin-like 4 | SEQ ID NOS: 815-822 |
| ANGPTL5 | Angiopoietin-like 5 | SEQ ID NOS: 823-824 |
| ANGPTL6 | Angiopoietin-like 6 | SEQ ID NOS: 825-827 |
| ANGPTL7 | Angiopoietin-like 7 | SEQ ID NO: 828 |
| ANK1 | Ankyrin 1, erythrocytic | SEQ ID NOS: 833-843 |
| ANKDD1A | Ankyrin repeat and death domain containing | SEQ ID NOS: 844-850 |
| | 1A | |
| ANKRD54 | Ankyrin repeat domain 54 | SEQ ID NOS: 851-859 |
| ANKRD60 | Ankyrin repeat domain 60 | SEQ ID NO: 860 |
| ANO7 | Anoctamin 7 | SEQ ID NOS: 861-864 |
| ANO1 | #N/A | SEQ ID NO: 865 |
| ANTXR1 | Anthrax toxin receptor 1 | SEQ ID NOS: 866-869 |
| AOAH | Acyloxyacyl hydrolase (neutrophil) | SEQ ID NOS: 870-874 |
| AOC1 | Amine oxidase, copper containing 1 | SEQ ID NOS: 875-880 |
| AOC2 | Amine oxidase, copper containing 2 (retina- | SEQ ID NOS: 881-882 |
| | specific) | |
| AOC3 | Amine oxidase, copper containing 3 | SEQ ID NOS: 883-889 |
| AP000721.4 | | SEQ ID NO: 890 |
| APBB1 | Amyloid beta (A4) precursor protein- | SEQ ID NOS: 891-907 |
| | binding, family B, member 1 (Fe65) | |
| APCDD1 | Adenomatosis polyposis coli down- | SEQ ID NOS: 908-913 |
| | regulated 1 | |
| APCS | Amyloid P component, serum | SEQ ID NO: 914 |
| APELA | Apelin receptor early endogenous ligand | SEQ ID NOS: 915-917 |
| APLN | Apelin | SEQ ID NO: 918 |
| APLP2 | Amyloid beta (A4) precursor-like protein 2 | SEQ ID NOS: 919-928 |
| APOA1 | Apolipoprotein A-I | SEQ ID NOS: 929-933 |
| APOA1BP | Apolipoprotein A-I binding protein | SEQ ID NOS: 9177- |
| | | 9179 |
| APOA2 | Apolipoprotein A-II | SEQ ID NOS: 934-942 |
| APOA4 | Apolipoprotein A-IV | SEQ ID NO: 943 |
| APOA5 | Apolipoprotein A-V | SEQ ID NOS: 944-946 |
| APOB | Apolipoprotein B | SEQ ID NOS: 947-948 |
| APOC1 | Apolipoprotein C-I | SEQ ID NOS: 949-957 |
| APOC2 | Apolipoprotein C-II | SEQ ID NOS: 958-962 |
| APOC3 | Apolipoprotein C-III | SEQ ID NOS: 963-966 |
| APOC4 | Apolipoprotein C-IV | SEQ ID NOS: 967-968 |
| APOC4- | APOC4-APOC2 readthrough (NMD | SEQ ID NOS: 969-970 |
| APOC2 | candidate) | |
| APOD | Apolipoprotein D | SEQ ID NOS: 971-974 |
| APOE | Apolipoprotein E | SEQ ID NOS: 975-978 |
| APOF | Apolipoprotein F | SEQ ID NO: 979 |
| APOH | Apolipoprotein H (beta-2-glycoprotein I) | SEQ ID NOS: 980-983 |
| APOL1 | Apolipoprotein L, 1 | SEQ ID NOS: 984-994 |
| APOL3 | Apolipoprotein L, 3 | SEQ ID NOS: 995-1009 |
| APOM | Apolipoprotein M | SEQ ID NOS: 1010- |
| | | 1012 |
| APOOL | Apolipoprotein O-like | SEQ ID NOS: 1013- |
| | | 1015 |
| ARCN1 | Archain 1 | SEQ ID NOS: 1016- |
| | | 1020 |
| ARFIP2 | ADP-ribosylation factor interacting protein | SEQ ID NOS: 1021- |
| | 2 | 1027 |
| ARHGAP36 | Rho GTPase activating protein 36 | SEQ ID NOS: 1028- |
| | | 1033 |
| ARHGAP6 | Rho GTPase activating protein 6 | SEQ ID NOS: 1043- |
| | | 1048 |
| ARHGEF4 | Rho guanine nucleotide exchange factor | SEQ ID NOS: 1049- |
| | (GEF) 4 | 1059 |
| ARL16 | ADP-ribosylation factor-like 16 | SEQ ID NOS: 1060- |
| | | 1068 |
| ARMC5 | Armadillo repeat containing 5 | SEQ ID NOS: 1069- |
| | | 1075 |
| ARNTL | Aryl hydrocarbon receptor nuclear | SEQ ID NOS: 1076- |
| | translocator-like | 1090 |
| ARSA | Arylsulfatase A | SEQ ID NOS: 1091- |
| | | 1096 |
| ARSB | Arylsulfatase B | SEQ ID NOS: 1097- |
| | | 1100 |
| ARSE | Arylsulfatase E (chondrodysplasia punctata | SEQ ID NOS: 1101- |
| | 1) | 1104 |
| ARSG | Arylsulfatase G | SEQ ID NOS: 1105- |
| | | 1108 |
| ARSI | Arylsulfatase family, member I | SEQ ID NOS: 1109- |
| | | 1111 |
| ARSK | Arylsulfatase family, member K | SEQ ID NOS: 1112- |
| | | 1116 |
| ART3 | ADP-ribosyltransferase 3 | SEQ ID NOS: 1117- |
| | | 1124 |
| ART4 | ADP-ribosyltransferase 4 (Dombrock blood | SEQ ID NOS: 1125- |
| | group) | 1128 |
| ART5 | ADP-ribosyltransferase 5 | SEQ ID NOS: 1129- |
| | | 1133 |
| ARTN | Artemin | SEQ ID NOS: 1134- |
| | | 1144 |
| ASAH1 | N-acylsphingosine amidohydrolase (acid | SEQ ID NOS: 1145- |
| | ceramidase) 1 | 1195 |
| ASAH2 | N-acylsphingosine amidohydrolase (non- | SEQ ID NOS: 1196- |
| | lysosomal ceramidase) 2 | 1201 |
| ASCL1 | Achaete-scute family bHLH transcription | SEQ ID NO: 1202 |
| | factor 1 | |
| ASIP | Agouti signaling protein | SEQ ID NOS: 1203- |
| | | 1204 |
| ASPN | Asporin | SEQ ID NOS: 1205- |
| | | 1206 |
| ASTL | Astacin-like metallo-endopeptidase (M12 | SEQ ID NO: 1207 |
| | family) | |
| ATAD5 | ATPase family, AAA domain containing 5 | SEQ ID NOS: 1208- |
| | | 1209 |
| ATAT1 | Alpha tubulin acetyltransferase 1 | SEQ ID NOS: 1210- |
| | | 1215 |
| ATG2A | Autophagy related 2A | SEQ ID NOS: 1216- |
| | | 1218 |
| ATG5 | Autophagy related 5 | SEQ ID NOS: 1219- |
| | | 1227 |
| ATMIN | ATM interactor | SEQ ID NOS: 1228- |
| | | 1231 |
| ATP13A1 | ATPase type 13A1 | SEQ ID NOS: 1232- |
| | | 1234 |
| ATP5F1 | ATP synthase, H+ transporting, | SEQ ID NOS: 1235- |
| | mitochondrial Fo complex, subunit Bl | 1236 |
| ATP6AP1 | ATPase, H+ transporting, lysosomal | SEQ ID NOS: 1237- |
| | accessory protein 1 | 1244 |
| ATP6AP2 | ATPase, H+ transporting, lysosomal | SEQ ID NOS: 1245- |
| | accessory protein 2 | 1267 |
| ATPAF1 | ATP synthase mitochondrial F1 complex | SEQ ID NOS: 1268- |
| | assembly factor 1 | 1278 |
| AUH | AU RNA binding protein/enoyl-CoA | SEQ ID NOS: 1279- |
| | hydratase | 1280 |
| AVP | Arginine vasopressin | SEQ ID NO: 1281 |
| AXIN2 | Axin 2 | SEQ ID NOS: 1282- |
| | | 1289 |
| AZGP1 | Alpha-2-glycoprotein 1, zinc-binding | SEQ ID NOS: 1290- |
| | | 1292 |
| AZU1 | Azurocidin 1 | SEQ ID NOS: 1293- |
| | | 1294 |
| B2M | Beta-2-microglobulin | SEQ ID NOS: 1295- |
| | | 1301 |
| B3GALNT1 | Beta-1,3-N-acetylgalactosaminyltransferase | SEQ ID NOS: 1302- |
| | 1 (globoside blood group) | 1314 |
| B3GALNT2 | Beta-1,3-N-acetylgalactosaminyltransferase | SEQ ID NOS: 1315- |
| | 2 | 1317 |
| B3GALT1 | UDP-Gal:betaGlcNAc beta 1,3- | SEQ ID NO: 1318 |
| | galactosyltransferase, polypeptide 1 | |
| B3GALT4 | UDP-Gal:betaGlcNAc beta 1,3- | SEQ ID NO: 1319 |
| | galactosyltransferase, polypeptide 4 | |
| B3GALT5 | UDP-Gal:betaGlcNAc beta 1,3- | SEQ ID NOS: 1320- |
| | galactosyltransferase, polypeptide 5 | 1324 |
| B3GALT6 | UDP-Gal:betaGal beta 1,3- | SEQ ID NO: 1325 |
| | galactosyltransferase polypeptide 6 | |
| B3GAT3 | Beta-1,3-glucuronyltransferase 3 | SEQ ID NOS: 1326- |
| | | 1330 |
| B3GLCT | Beta 3-glucosyltransferase | SEQ ID NO: 1331 |
| B3GNT3 | UDP-GlcNAc:betaGal beta-1,3-N- | SEQ ID NOS: 1332- |
| | acetylglucosaminyltransferase 3 | 1335 |
| B3GNT4 | UDP-GlcNAc:betaGal beta-1,3-N- | SEQ ID NOS: 1336- |
| | acetylglucosaminyltransferase 4 | 1339 |
| B3GNT6 | UDP-GlcNAc:betaGal beta-1,3-N- | SEQ ID NOS: 1340- |
| | acetylglucosaminyltransferase 6 | 1341 |
| B3GNT7 | UDP-GlcNAc:betaGal beta-1,3-N- | SEQ ID NO: 1342 |
| | acetylglucosaminyltransferase 7 | |
| B3GNT8 | UDP-GlcNAc:betaGal beta-1,3-N- | SEQ ID NO: 1343 |
| | acetylglucosaminyltransferase 8 | |
| B3GNT9 | UDP-GlcNAc:betaGal beta-1,3-N- | SEQ ID NO: 1344 |
| | acetylglucosaminyltransferase 9 | |
| B4GALNT1 | Beta-1,4-N-acetyl-galactosaminyl | SEQ ID NOS: 1345- |
| | transferase 1 | 1356 |
| B4GALNT3 | Beta-1,4-N-acetyl-galactosaminyl | SEQ ID NOS: 1357- |
| | transferase 3 | 1358 |
| B4GALNT4 | Beta-1,4-N-acetyl-galactosaminyl | SEQ ID NOS: 1359- |
| | transferase 4 | 1361 |
| B4GALT4 | UDP-Gal:betaGlcNAc beta 1,4- | SEQ ID NOS: 1362- |
| | galactosyltransferase, polypeptide 4 | 1374 |
| B4GALT5 | UDP-Gal:betaGlcNAc beta 1,4- | SEQ ID NO: 1375 |
| | galactosyltransferase, polypeptide 5 | |
| B4GALT6 | UDP-Gal:betaGlcNAc beta 1,4- | SEQ ID NOS: 1376- |
| | galactosyltransferase, polypeptide 6 | 1379 |
| B4GAT1 | Beta-1,4-glucuronyltransferase 1 | SEQ ID NO: 1380 |
| B9D1 | B9 protein domain 1 | SEQ ID NOS: 1381- |
| | | 1397 |
| BACE2 | Beta-site APP-cleaving enzyme 2 | SEQ ID NOS: 1398- |
| | | 1400 |
| BAGE5 | B melanoma antigen family, member 5 | SEQ ID NO: 1401 |
| BCAM | Basal cell adhesion molecule (Lutheran | SEQ ID NOS: 1402- |
| | blood group) | 1405 |
| BCAN | Brevican | SEQ ID NOS: 1406- |
| | | 1412 |
| BCAP29 | B-cell receptor-associated protein 29 | SEQ ID NOS: 1413- |
| | | 1425 |
| BCAR1 | Breast cancer anti-estrogen resistance 1 | SEQ ID NOS: 1426- |
| | | 1443 |
| BCHE | Butyrylcholinesterase | SEQ ID NOS: 1444- |
| | | 1448 |
| BCKDHB | Branched chain keto acid dehydrogenase | SEQ ID NOS: 1449- |
| | E1, beta polypeptide | 1451 |
| BDNF | Brain-derived neurotrophic factor | SEQ ID NOS: 1452- |
| | | 1469 |
| BGLAP | Bone gamma-carboxyglutamate (gla) | SEQ ID NO: 1470 |
| | protein | |
| BGN | Biglycan | SEQ ID NOS: 1471- |
| | | 1472 |
| BLVRB | Biliverdin reductase B | SEQ ID NOS: 1473- |
| | | 1477 |
| BMP1 | Bone morphogenetic protein 1 | SEQ ID NOS: 1478- |
| | | 1489 |
| BMP10 | Bone morphogenetic protein 10 | SEQ ID NO: 1490 |
| BMP15 | Bone morphogenetic protein 15 | SEQ ID NO: 1491 |
| BMP2 | Bone morphogenetic protein 2 | SEQ ID NO: 1492 |
| BMP3 | Bone morphogenetic protein 3 | SEQ ID NO: 1493 |
| BMP4 | Bone morphogenetic protein 4 | SEQ ID NOS: 1494- |
| | | 1501 |
| BMP6 | Bone morphogenetic protein 6 | SEQ ID NO: 1502 |
| BMP7 | Bone morphogenetic protein 7 | SEQ ID NOS: 1503- |
| | | 1506 |
| BMP8A | Bone morphogenetic protein 8a | SEQ ID NO: 1507 |
| BMP8B | Bone morphogenetic protein 8b | SEQ ID NO: 1508 |
| BMPER | BMP binding endothelial regulator | SEQ ID NOS: 1509- |
| | | 1512 |
| BNC1 | Basonuclin 1 | SEQ ID NOS: 1513- |
| | | 1514 |
| BOC | BOC cell adhesion associated, oncogene | SEQ ID NOS: 1515- |
| | regulated | 1525 |
| BOD1 | Biorientation of chromosomes in cell | SEQ ID NOS: 1526- |
| | division 1 | 1530 |
| BOLA1 | BolA family member 1 | SEQ ID NOS: 1531- |
| | | 1533 |
| BPI | Bactericidal/permeability-increasing protein | SEQ ID NOS: 1534- |
| | | 1537 |
| BPIFA1 | BPI fold containing family A, member 1 | SEQ ID NOS: 1538- |
| | | 1541 |
| BPIFA2 | BPI fold containing family A, member 2 | SEQ ID NOS: 1542- |
| | | 1543 |
| BPIFA3 | BPI fold containing family A, member 3 | SEQ ID NOS: 1544- |
| | | 1545 |
| BPIFB1 | BPI fold containing family B, member 1 | SEQ ID NOS: 1546- |
| | | 1547 |
| BPIFB2 | BPI fold containing family B, member 2 | SEQ ID NO: 1548 |
| BPIFB3 | BPI fold containing family B, member 3 | SEQ ID NO: 1549 |
| BPIFB4 | BPI fold containing family B, member 4 | SEQ ID NOS: 1550- |
| | | 1551 |
| BPIFB6 | BPI fold containing family B, member 6 | SEQ ID NOS: 1552- |
| | | 1553 |
| BPIFC | BPI fold containing family C | SEQ ID NOS: 1554- |
| | | 1557 |
| BRF1 | BRF1, RNA polymerase III transcription | SEQ ID NOS: 1558- |
| | initiation factor 90 kDa subunit | 1573 |
| BRINP1 | Bone morphogenetic protein/retinoic acid | SEQ ID NOS: 1574- |
| | inducible neural-specific 1 | 1575 |
| BRINP2 | Bone morphogenetic protein/retinoic acid | SEQ ID NO: 1576 |
| | inducible neural-specific 2 | |
| BRINP3 | Bone morphogenetic protein/retinoic acid | SEQ ID NOS: 1577- |
| | inducible neural-specific 3 | 1579 |
| BSG | Basigin (Ok blood group) | SEQ ID NOS: 1580- |
| | | 1590 |
| BSPH1 | Binder of sperm protein homolog 1 | SEQ ID NO: 1591 |
| BST1 | Bone marrow stromal cell antigen 1 | SEQ ID NOS: 1592- |
| | | 1596 |
| BTBD17 | BTB (POZ) domain containing 17 | SEQ ID NO: 1597 |
| BTD | Biotinidase | SEQ ID NOS: 1598- |
| | | 1607 |
| BTN2A2 | Butyrophilin, subfamily 2, member A2 | SEQ ID NOS: 1608- |
| | | 1621 |
| BTN3A1 | Butyrophilin, subfamily 3, member A1 | SEQ ID NOS: 1622- |
| | | 1628 |
| BTN3A2 | Butyrophilin, subfamily 3, member A2 | SEQ ID NOS: 1629- |
| | | 1639 |
| BTN3A3 | Butyrophilin, subfamily 3, member A3 | SEQ ID NOS: 1640- |
| | | 1648 |
| C10orf10 | Chromosome 10 open reading frame 10 | SEQ ID NOS: 4169- |
| | | 4170 |
| C10orf99 | Chromosome 10 open reading frame 99 | SEQ ID NO: 1650 |
| C11orf1 | Chromosome 11 open reading frame 1 | SEQ ID NOS: 1651- |
| | | 1655 |
| C11orf24 | Chromosome 11 open reading frame 24 | SEQ ID NOS: 1656- |
| | | 1658 |
| C11orf45 | Chromosome 11 open reading frame 45 | SEQ ID NOS: 1659- |
| | | 1660 |
| C11orf94 | Chromosome 11 open reading frame 94 | SEQ ID NO: 1661 |
| C12orf10 | Chromosome 12 open reading frame 10 | SEQ ID NOS: 1662- |
| | | 1665 |
| C12orf49 | Chromosome 12 open reading frame 49 | SEQ ID NOS: 1666- |
| | | 1669 |
| C12orf73 | Chromosome 12 open reading frame 73 | SEQ ID NOS: 1670- |
| | | 1679 |
| C12orf76 | Chromosome 12 open reading frame 76 | SEQ ID NOS: 1680- |
| | | 1687 |
| C14orf80 | Chromosome 14 open reading frame 80 | SEQ ID NOS: 13083- |
| | | 13096 |
| C14orf93 | Chromosome 14 open reading frame 93 | SEQ ID NOS: 1688- |
| | | 1703 |
| C16orf89 | Chromosome 16 open reading frame 89 | SEQ ID NOS: 1704- |
| | | 1706 |
| C16orf90 | Chromosome 16 open reading frame 90 | SEQ ID NOS: 1707- |
| | | 1708 |
| C17orf67 | Chromosome 17 open reading frame 67 | SEQ ID NO: 1709 |
| C17orf75 | Chromosome 17 open reading frame 75 | SEQ ID NOS: 1710- |
| | | 1718 |
| C17orf99 | Chromosome 17 open reading frame 99 | SEQ ID NOS: 1719- |
| | | 1721 |
| C18orf54 | Chromosome 18 open reading frame 54 | SEQ ID NOS: 1722- |
| | | 1726 |
| C19orf47 | Chromosome 19 open reading frame 47 | SEQ ID NOS: 1727- |
| | | 1734 |
| C19orf70 | Chromosome 19 open reading frame 70 | SEQ ID NOS: 1735- |
| | | 1738 |
| C19orf80 | Chromosome 19 open reading frame 80 | SEQ ID NOS: 829-832 |
| C1GALT1 | Core 1 synthase, glycoprotein-N- | SEQ ID NOS: 1739- |
| | acetylgalactosamine 3-beta- | 1743 |
| | galactosyltransferase 1 | |
| C1orf127 | Chromosome 1 open reading frame 127 | SEQ ID NOS: 1744- |
| | | 1747 |
| C1orf159 | Chromosome 1 open reading frame 159 | SEQ ID NOS: 1748- |
| | | 1760 |
| C1orf198 | Chromosome 1 open reading frame 198 | SEQ ID NOS: 1761- |
| | | 1765 |
| C1orf234 | Chromosome 1 open reading frame 234 | SEQ ID NOS: 13118- |
| | | 13120 |
| C1orf54 | Chromosome 1 open reading frame 54 | SEQ ID NOS: 1766- |
| | | 1768 |
| C1orf56 | Chromosome 1 open reading frame 56 | SEQ ID NO: 1769 |
| C1QA | Complement component 1, q | SEQ ID NOS: 1770- |
| | subcomponent, A chain | 1772 |
| C1QB | Complement component 1, q | SEQ ID NOS: 1773- |
| | subcomponent, B chain | 1776 |
| C1QC | Complement component 1, q | SEQ ID NOS: 1777- |
| | subcomponent, C chain | 1779 |
| C1QL1 | Complement component 1, q | SEQ ID NO: 1780 |
| | subcomponent-like 1 | |
| C1QL2 | Complement component 1, q | SEQ ID NO: 1781 |
| | subcomponent-like 2 | |
| C1QL3 | Complement component 1, q | SEQ ID NOS: 1782- |
| | subcomponent-like 3 | 1783 |
| C1QL4 | Complement component 1, q | SEQ ID NO: 1784 |
| | subcomponent-like 4 | |
| C1QTNF1 | C1q and tumor necrosis factor related | SEQ ID NOS: 1785- |
| | protein 1 | 1794 |
| C1QTNF2 | C1q and tumor necrosis factor related | SEQ ID NO: 1796 |
| | protein 2 | |
| C1QTNF3 | C1q and tumor necrosis factor related | SEQ ID NOS: 1797- |
| | protein 3 | 1798 |
| C1QTNF4 | C1q and tumor necrosis factor related | SEQ ID NOS: 1799- |
| | protein 4 | 1800 |
| C1QTNF5 | C1q and tumor necrosis factor related | SEQ ID NOS: 1801- |
| | protein 5 | 1803 |
| C1QTNF7 | C1q and tumor necrosis factor related | SEQ ID NOS: 1804- |
| | protein 7 | 1808 |
| C1QTNF8 | C1q and tumor necrosis factor related | SEQ ID NOS: 1809- |
| | protein 8 | 1810 |
| C1QTNF9 | C1q and tumor necrosis factor related | SEQ ID NOS: 1811- |
| | protein 9 | 1812 |
| C1QTNF9B | C1q and tumor necrosis factor related | SEQ ID NOS: 1813- |
| | protein 9B | 1815 |
| C1R | Complement component 1, r subcomponent | SEQ ID NOS: 1816- |
| | | 1824 |
| C1RL | Complement component 1, r subcomponent- | SEQ ID NOS: 1825- |
| | like | 1833 |
| C1S | Complement component 1, s subcomponent | SEQ ID NOS: 1834- |
| | | 1843 |
| C2 | Complement component 2 | SEQ ID NOS: 1844- |
| | | 1858 |
| C21orf33 | Chromosome 21 open reading frame 33 | SEQ ID NOS: 1859- |
| | | 1867 |
| C21orf62 | Chromosome 21 open reading frame 62 | SEQ ID NOS: 1868- |
| | | 1871 |
| C22orf15 | Chromosome 22 open reading frame 15 | SEQ ID NOS: 1872- |
| | | 1874 |
| C22orf46 | Chromosome 22 open reading frame 46 | SEQ ID NO: 1875 |
| C2CD2 | C2 calcium-dependent domain containing 2 | SEQ ID NOS: 1876- |
| | | 1878 |
| C2orf40 | Chromosome 2 open reading frame 40 | SEQ ID NOS: 1879- |
| | | 1881 |
| C2orf66 | Chromosome 2 open reading frame 66 | SEQ ID NO: 1882 |
| C2orf69 | Chromosome 2 open reading frame 69 | SEQ ID NO: 1883 |
| C2orf78 | Chromosome 2 open reading frame 78 | SEQ ID NO: 1884 |
| C3 | Complement component 3 | SEQ ID NOS: 1885- |
| | | 1889 |
| C3orf33 | Chromosome 3 open reading frame 33 | SEQ ID NOS: 1890- |
| | | 1894 |
| C3orf58 | Chromosome 3 open reading frame 58 | SEQ ID NOS: 1895- |
| | | 1898 |
| C4A | Complement component 4A (Rodgers blood | SEQ ID NOS: 1899- |
| | group) | 1900 |
| C4B | Complement component 4B (Chido blood | SEQ ID NOS: 1901- |
| | group) | 1902 |
| C4BPA | Complement component 4 binding protein, | SEQ ID NOS: 1903- |
| | alpha | 1905 |
| C4BPB | Complement component 4 binding protein, | SEQ ID NOS: 1906- |
| | beta | 1910 |
| C4orf26 | Chromosome 4 open reading frame 26 | SEQ ID NOS: 9751- |
| | | 9754 |
| C4orf48 | Chromosome 4 open reading frame 48 | SEQ ID NOS: 1911- |
| | | 1912 |
| C5 | Complement component 5 | SEQ ID NO: 1913 |
| C5orf46 | Chromosome 5 open reading frame 46 | SEQ ID NOS: 1914- |
| | | 1915 |
| C6 | Complement component 6 | SEQ ID NOS: 1916- |
| | | 1919 |
| C6orf120 | Chromosome 6 open reading frame 120 | SEQ ID NO: 1920 |
| C6orf15 | Chromosome 6 open reading frame 15 | SEQ ID NO: 1921 |
| C6orf25 | Chromosome 6 open reading frame 25 | SEQ ID NOS: 8832- |
| | | 8839 |
| C6orf58 | Chromosome 6 open reading frame 58 | SEQ ID NO: 1922 |
| C7 | Complement component 7 | SEQ ID NO: 1923 |
| C7orf57 | Chromosome 7 open reading frame 57 | SEQ ID NOS: 1924- |
| | | 1928 |
| C7orf73 | Chromosome 7 open reading frame 73 | SEQ ID NOS: 12924- |
| | | 12925 |
| C8A | Complement component 8, alpha | SEQ ID NO: 1929 |
| | polypeptide | |
| C8B | Complement component 8, beta polypeptide | SEQ ID NOS: 1930- |
| | | 1932 |
| C8G | Complement component 8, gamma | SEQ ID NOS: 1933- |
| | polypeptide | 1934 |
| C9 | Complement component 9 | SEQ ID NO: 1935 |
| C9orf47 | Chromosome 9 open reading frame 47 | SEQ ID NOS: 1936- |
| | | 1938 |
| CA10 | Carbonic anhydrase X | SEQ ID NOS: 1939- |
| | | 1945 |
| CA11 | Carbonic anhydrase XI | SEQ ID NOS: 1946- |
| | | 1947 |
| CA6 | Carbonic anhydrase VI | SEQ ID NOS: 1948- |
| | | 1952 |
| CA9 | Carbonic anhydrase IX | SEQ ID NOS: 1953- |
| | | 1954 |
| CABLES1 | Cdk5 and Abl enzyme substrate 1 | SEQ ID NOS: 1955- |
| | | 1960 |
| CABP1 | Calcium binding protein 1 | SEQ ID NOS: 1961- |
| | | 1964 |
| CACNA2D1 | Calcium channel, voltage-dependent, alpha | SEQ ID NOS: 1965- |
| | 2/delta subunit 1 | 1968 |
| CACNA2D4 | Calcium channel, voltage-dependent, alpha | SEQ ID NOS: 1969- |
| | 2/delta subunit 4 | 1982 |
| CADM3 | Cell adhesion molecule 3 | SEQ ID NOS: 1983- |
| | | 1985 |
| CALCA | Calcitonin-related polypeptide alpha | SEQ ID NOS: 1986- |
| | | 1990 |
| CALCB | Calcitonin-related polypeptide beta | SEQ ID NOS: 1991- |
| | | 1993 |
| CALCR | Calcitonin receptor | SEQ ID NOS: 1994- |
| | | 2000 |
| CALCRL | Calcitonin receptor-like | SEQ ID NOS: 2001- |
| | | 2005 |
| CALR | Calreticulin | SEQ ID NOS: 2011- |
| | | 2014 |
| CALR3 | Calreticulin 3 | SEQ ID NOS: 2015- |
| | | 2016 |
| CALU | Calumenin | SEQ ID NOS: 2017- |
| | | 2022 |
| CAMK2D | Calcium/calmodulin-dependent protein | SEQ ID NOS: 2023- |
| | kinase II delta | 2034 |
| CAMP | Cathelicidin antimicrobial peptide | SEQ ID NO: 2035 |
| CANX | Calnexin | SEQ ID NOS: 2036- |
| | | 2050 |
| CARKD | Carbohydrate kinase domain containing | SEQ ID NOS: 9175- |
| | | 9176 |
| CARM1 | Coactivator-associated arginine | SEQ ID NOS: 2051- |
| | methyltransferase 1 | 2058 |
| CARNS1 | Carnosine synthase 1 | SEQ ID NOS: 2059- |
| | | 2061 |
| CARTPT | CART prepropeptide | SEQ ID NO: 2062 |
| CASQ1 | Calsequestrin 1 (fast-twitch, skeletal | SEQ ID NOS: 2063- |
| | muscle) | 2064 |
| CASQ2 | Calsequestrin 2 (cardiac muscle) | SEQ ID NO: 2065 |
| CATSPERG | Catsper channel auxiliary subunit gamma | SEQ ID NOS: 2066- |
| | | 2073 |
| CBLN1 | Cerebellin 1 precursor | SEQ ID NOS: 2074- |
| | | 2076 |
| CBLN2 | Cerebellin 2 precursor | SEQ ID NOS: 2077- |
| | | 2080 |
| CBLN3 | Cerebellin 3 precursor | SEQ ID NOS: 2081- |
| | | 2082 |
| CBLN4 | Cerebellin 4 precursor | SEQ ID NO: 2083 |
| CCBE1 | Collagen and calcium binding EGF domains | SEQ ID NOS: 2084- |
| | 1 | 2086 |
| CCDC108 | Coiled-coil domain containing 108 | SEQ ID NOS: 2659- |
| | | 2668 |
| CCDC112 | Coiled-coil domain containing 112 | SEQ ID NOS: 2087- |
| | | 2090 |
| CCDC129 | Coiled-coil domain containing 129 | SEQ ID NOS: 2091- |
| | | 2098 |
| CCDC134 | Coiled-coil domain containing 134 | SEQ ID NOS: 2099- |
| | | 2100 |
| CCDC149 | Coiled-coil domain containing 149 | SEQ ID NOS: 2101- |
| | | 2104 |
| CCDC3 | Coiled-coil domain containing 3 | SEQ ID NOS: 2105- |
| | | 2106 |
| CCDC80 | Coiled-coil domain containing 80 | SEQ ID NOS: 2107- |
| | | 2110 |
| CCDC85A | Coiled-coil domain containing 85A | SEQ ID NO: 2111 |
| CCDC88B | Coiled-coil domain containing 88B | SEQ ID NOS: 2112- |
| | | 2114 |
| CCER2 | Coiled-coil glutamate-rich protein 2 | SEQ ID NOS: 2115- |
| | | 2116 |
| CCK | Cholecystokinin | SEQ ID NOS: 2117- |
| | | 2119 |
| CCL1 | Chemokine (C-C motif) ligand 1 | SEQ ID NO: 2120 |
| CCL11 | Chemokine (C-C motif) ligand 11 | SEQ ID NO: 2121 |
| CCL13 | Chemokine (C-C motif) ligand 13 | SEQ ID NOS: 2122- |
| | | 2123 |
| CCL14 | Chemokine (C-C motif) ligand 14 | SEQ ID NOS: 2124- |
| | | 2127 |
| CCL15 | Chemokine (C-C motif) ligand 15 | SEQ ID NOS: 2128- |
| | | 2129 |
| CCL16 | Chemokine (C-C motif) ligand 16 | SEQ ID NOS: 2130- |
| | | 2132 |
| CCL17 | Chemokine (C-C motif) ligand 17 | SEQ ID NOS: 2133- |
| | | 2134 |
| CCL18 | Chemokine (C-C motif) ligand 18 | SEQ ID NO: 2135 |
| | (pulmonary and activation-regulated) | |
| CCL19 | Chemokine (C-C motif) ligand 19 | SEQ ID NOS: 2136- |
| | | 2137 |
| CCL2 | Chemokine (C-C motif) ligand 2 | SEQ ID NOS: 2138- |
| | | 2139 |
| CCL20 | Chemokine (C-C motif) ligand 20 | SEQ ID NOS: 2140- |
| | | 2142 |
| CCL21 | Chemokine (C-C motif) ligand 21 | SEQ ID NOS: 2143- |
| | | 2144 |
| CCL22 | Chemokine (C-C motif) ligand 22 | SEQ ID NO: 2145 |
| CCL23 | Chemokine (C-C motif) ligand 23 | SEQ ID NOS: 2146- |
| | | 2148 |
| CCL24 | Chemokine (C-C motif) ligand 24 | SEQ ID NOS: 2149- |
| | | 2150 |
| CCL25 | Chemokine (C-C motif) ligand 25 | SEQ ID NOS: 2151- |
| | | 2154 |
| CCL26 | Chemokine (C-C motif) ligand 26 | SEQ ID NOS: 2155- |
| | | 2156 |
| CCL27 | Chemokine (C-C motif) ligand 27 | SEQ ID NO: 2157 |
| CCL28 | Chemokine (C-C motif) ligand 28 | SEQ ID NOS: 2158- |
| | | 2160 |
| CCL3 | Chemokine (C-C motif) ligand 3 | SEQ ID NO: 2161 |
| CCL3L3 | Chemokine (C-C motif) ligand 3-like 3 | SEQ ID NO: 2162 |
| CCL4 | Chemokine (C-C motif) ligand 4 | SEQ ID NOS: 2163- |
| | | 2164 |
| CCL4L2 | Chemokine (C-C motif) ligand 4-like 2 | SEQ ID NOS: 2165- |
| | | 2174 |
| CCL5 | Chemokine (C-C motif) ligand 5 | SEQ ID NOS: 2175- |
| | | 2177 |
| CCL7 | Chemokine (C-C motif) ligand 7 | SEQ ID NOS: 2178- |
| | | 2180 |
| CCL8 | Chemokine (C-C motif) ligand 8 | SEQ ID NO: 2181 |
| CCNB1IP1 | Cyclin Bl interacting protein 1, E3 | SEQ ID NOS: 2182- |
| | ubiquitin protein ligase | 2193 |
| CCNL1 | Cyclin L1 | SEQ ID NOS: 2194- |
| | | 2202 |
| CCNL2 | Cyclin L2 | SEQ ID NOS: 2203- |
| | | 2210 |
| CD14 | CD14 molecule | SEQ ID NOS: 2211- |
| | | 2215 |
| CD160 | CD160 molecule | SEQ ID NOS: 2216- |
| | | 2220 |
| CD164 | CD164 molecule, sialomucin | SEQ ID NOS: 2221- |
| | | 2226 |
| CD177 | CD177 molecule | SEQ ID NOS: 2227- |
| | | 2229 |
| CD1E | CD1e molecule | SEQ ID NOS: 2230- |
| | | 2243 |
| CD2 | CD2 molecule | SEQ ID NOS: 2244- |
| | | 2245 |
| CD200 | CD200 molecule | SEQ ID NOS: 2246- |
| | | 2252 |
| CD200R1 | CD200 receptor 1 | SEQ ID NOS: 2253- |
| | | 2257 |
| CD22 | CD22 molecule | SEQ ID NOS: 2258- |
| | | 2275 |
| CD226 | CD226 molecule | SEQ ID NOS: 2276- |
| | | 2283 |
| CD24 | CD24 molecule | SEQ ID NOS: 2284- |
| | | 2290 |
| CD276 | CD276 molecule | SEQ ID NOS: 2291- |
| | | 2306 |
| CD300A | CD300a molecule | SEQ ID NOS: 2307- |
| | | 2311 |
| CD300LB | CD300 molecule-like family member b | SEQ ID NOS: 2312- |
| | | 2313 |
| CD300LF | CD300 molecule-like family member f | SEQ ID NOS: 2314- |
| | | 2322 |
| CD300LG | CD300 molecule-like family member g | SEQ ID NOS: 2323- |
| | | 2328 |
| CD3D | CD3d molecule, delta (CD3-TCR complex) | SEQ ID NOS: 2329- |
| | | 2332 |
| CD4 | CD4 molecule | SEQ ID NOS: 2333- |
| | | 2335 |
| CD40 | CD40 molecule, TNF receptor superfamily | SEQ ID NOS: 2336- |
| | member 5 | 2339 |
| CD44 | CD44 molecule (Indian blood group) | SEQ ID NOS: 2340- |
| | | 2366 |
| CD48 | CD48 molecule | SEQ ID NOS: 2367- |
| | | 2369 |
| CD5 | CD5 molecule | SEQ ID NOS: 2370- |
| | | 2371 |
| CD55 | CD55 molecule, decay accelerating factor | SEQ ID NOS: 2372- |
| | for complement (Cromer blood group) | 2382 |
| CD59 | CD59 molecule, complement regulatory | SEQ ID NOS: 2383- |
| | protein | 2393 |
| CD5L | CD5 molecule-like | SEQ ID NO: 2394 |
| CD6 | CD6 molecule | SEQ ID NOS: 2395- |
| | | 2402 |
| CD68 | CD68 molecule | SEQ ID NOS: 2403- |
| | | 2406 |
| CD7 | CD7 molecule | SEQ ID NOS: 2407- |
| | | 2412 |
| CD79A | CD79a molecule, immunoglobulin- | SEQ ID NOS: 2413- |
| | associated alpha | 2415 |
| CD80 | CD80 molecule | SEQ ID NOS: 2416- |
| | | 2418 |
| CD86 | CD86 molecule | SEQ ID NOS: 2419- |
| | | 2425 |
| CD8A | CD8a molecule | SEQ ID NOS: 2426- |
| | | 2429 |
| CD8B | CD8b molecule | SEQ ID NOS: 2430- |
| | | 2435 |
| CD99 | CD99 molecule | SEQ ID NOS: 2436- |
| | | 2444 |
| CDC23 | Cell division cycle 23 | SEQ ID NOS: 2445- |
| | | 2449 |
| CDC40 | Cell division cycle 40 | SEQ ID NOS: 2450- |
| | | 2452 |
| CDC45 | Cell division cycle 45 | SEQ ID NOS: 2453- |
| | | 2459 |
| CDCP1 | CUB domain containing protein 1 | SEQ ID NOS: 2460- |
| | | 2461 |
| CDCP2 | CUB domain containing protein 2 | SEQ ID NOS: 2462- |
| | | 2463 |
| CDH1 | Cadherin 1, type 1 | SEQ ID NOS: 2464- |
| | | 2471 |
| CDH11 | Cadherin 11, type 2, OB-cadherin | SEQ ID NOS: 2472- |
| | (osteoblast) | 2481 |
| CDH13 | Cadherin 13 | SEQ ID NOS: 2482- |
| | | 2491 |
| CDH17 | Cadherin 17, LI cadherin (liver-intestine) | SEQ ID NOS: 2492- |
| | | 2496 |
| CDH18 | Cadherin 18, type 2 | SEQ ID NOS: 2497- |
| | | 2503 |
| CDH19 | Cadherin 19, type 2 | SEQ ID NOS: 2504- |
| | | 2508 |
| CDH23 | Cadherin-related 23 | SEQ ID NOS: 2509- |
| | | 2524 |
| CDH5 | Cadherin 5, type 2 (vascular endothelium) | SEQ ID NOS: 2525- |
| | | 2532 |
| CDHR1 | Cadherin-related family member 1 | SEQ ID NOS: 2533- |
| | | 2538 |
| CDHR4 | Cadherin-related family member 4 | SEQ ID NOS: 2539- |
| | | 2543 |
| CDHR5 | Cadherin-related family member 5 | SEQ ID NOS: 2544- |
| | | 2550 |
| CDKN2A | Cyclin-dependent kinase inhibitor 2A | SEQ ID NOS: 2551- |
| | | 2561 |
| CDNF | Cerebral dopamine neurotrophic factor | SEQ ID NOS: 2562- |
| | | 2563 |
| CDON | Cell adhesion associated, oncogene | SEQ ID NOS: 2564- |
| | regulated | 2571 |
| CDSN | Corneodesmosin | SEQ ID NO: 2572 |
| CEACAM16 | Carcinoembryonic antigen-related cell | SEQ ID NOS: 2573- |
| | adhesion molecule 16 | 2574 |
| CEACAM18 | Carcinoembryonic antigen-related cell | SEQ ID NO: 2575 |
| | adhesion molecule 18 | |
| CEACAM19 | Carcinoembryonic antigen-related cell | SEQ ID NOS: 2576- |
| | adhesion molecule 19 | 2582 |
| CEACAM5 | Carcinoembryonic antigen-related cell | SEQ ID NOS: 2583- |
| | adhesion molecule 5 | 2590 |
| CEACAM7 | Carcinoembryonic antigen-related cell | SEQ ID NOS: 2591- |
| | adhesion molecule 7 | 2593 |
| CEACAM8 | Carcinoembryonic antigen-related cell | SEQ ID NOS: 2594- |
| | adhesion molecule 8 | 2595 |
| CECR1 | Cat eye syndrome chromosome region, | SEQ ID NOS: 222-229 |
| | candidate 1 | |
| CECR5 | Cat eye syndrome chromosome region, | SEQ ID NOS: 6411- |
| | candidate 5 | 6413 |
| CEL | Carboxyl ester lipase | SEQ ID NO: 2596 |
| CELA2A | Chymotrypsin-like elastase family, member | SEQ ID NO: 2597 |
| | 2A | |
| CELA2B | Chymotrypsin-like elastase family, member | SEQ ID NOS: 2598- |
| | 2B | 2599 |
| CELA3A | Chymotrypsin-like elastase family, member | SEQ ID NOS: 2600- |
| | 3A | 2602 |
| CELA3B | Chymotrypsin-like elastase family, member | SEQ ID NOS: 2603- |
| | 3B | 2605 |
| CEMIP | Cell migration inducing protein, hyaluronan | SEQ ID NOS: 2606- |
| | binding | 2610 |
| CEP89 | Centrosomal protein 89 kDa | SEQ ID NOS: 2611- |
| | | 2616 |
| CER1 | Cerberus 1, DAN family BMP antagonist | SEQ ID NO: 2617 |
| CERCAM | Cerebral endothelial cell adhesion molecule | SEQ ID NOS: 2618- |
| | | 2625 |
| CERS1 | Ceramide synthase 1 | SEQ ID NOS: 2626- |
| | | 2630 |
| CES1 | Carboxylesterase 1 | SEQ ID NOS: 2631- |
| | | 2636 |
| CES3 | Carboxylesterase 3 | SEQ ID NOS: 2637- |
| | | 2641 |
| CES4A | Carboxylesterase 4A | SEQ ID NOS: 2642- |
| | | 2647 |
| CES5A | Carboxylesterase 5A | SEQ ID NOS: 2648- |
| | | 2655 |
| CETP | Cholesteryl ester transfer protein, plasma | SEQ ID NOS: 2656- |
| | | 2658 |
| CFB | Complement factor B | SEQ ID NOS: 2669- |
| | | 2673 |
| CFC1 | Cripto, FRL-1, cryptic family 1 | SEQ ID NOS: 2674- |
| | | 2676 |
| CFC1B | Cripto, FRL-1, cryptic family 1B | SEQ ID NOS: 2677- |
| | | 2679 |
| CFD | Complement factor D (adipsin) | SEQ ID NOS: 2680- |
| | | 2681 |
| CFDP1 | Craniofacial development protein 1 | SEQ ID NOS: 2682- |
| | | 2685 |
| CFH | Complement factor H | SEQ ID NOS: 2686- |
| | | 2688 |
| CFHR1 | Complement factor H-related 1 | SEQ ID NOS: 2689- |
| | | 2690 |
| CFHR2 | Complement factor H-related 2 | SEQ ID NOS: 2691- |
| | | 2692 |
| CFHR3 | Complement factor H-related 3 | SEQ ID NOS: 2693- |
| | | 2697 |
| CFHR4 | Complement factor H-related 4 | SEQ ID NOS: 2698- |
| | | 2701 |
| CFHR5 | Complement factor H-related 5 | SEQ ID NO: 2702 |
| CFI | Complement factor I | SEQ ID NOS: 2703- |
| | | 2707 |
| CFP | Complement factor properdin | SEQ ID NOS: 2708- |
| | | 2711 |
| CGA | Glycoprotein hormones, alpha polypeptide | SEQ ID NOS: 2712- |
| | | 2716 |
| CGB | Chorionic gonadotropin, beta polypeptide | SEQ ID NO: 2721 |
| CGB1 | Chorionic gonadotropin, beta polypeptide 1 | SEQ ID NOS: 2717- |
| | | 2718 |
| CGB2 | Chorionic gonadotropin, beta polypeptide 2 | SEQ ID NOS: 2719- |
| | | 2720 |
| CGB5 | Chorionic gonadotropin, beta polypeptide 5 | SEQ ID NO: 2722 |
| CGB7 | Chorionic gonadotropin, beta polypeptide 7 | SEQ ID NOS: 2723- |
| | | 2725 |
| CGB8 | Chorionic gonadotropin, beta polypeptide 8 | SEQ ID NO: 2726 |
| CGREF1 | Cell growth regulator with EF-hand domain | SEQ ID NOS: 2727- |
| | 1 | 2734 |
| CH507-9B2.3 | | SEQ ID NOS: 5532- |
| | | 5538 |
| CHAD | Chondroadherin | SEQ ID NOS: 2735- |
| | | 2737 |
| CHADL | Chondroadherin-like | SEQ ID NOS: 2738- |
| | | 2740 |
| CHEK2 | Checkpoint kinase 2 | SEQ ID NOS: 2741- |
| | | 2762 |
| CHGA | Chromogranin A | SEQ ID NOS: 2763- |
| | | 2765 |
| CHGB | Chromogranin B | SEQ ID NOS: 2766- |
| | | 2767 |
| CHI3L1 | Chitinase 3-like 1 (cartilage glycoprotein- | SEQ ID NOS: 2768- |
| | 39) | 2769 |
| CHI3L2 | Chitinase 3-like 2 | SEQ ID NOS: 2770- |
| | | 2783 |
| CHIA | Chitinase, acidic | SEQ ID NOS: 2784- |
| | | 2792 |
| CHID1 | Chitinase domain containing 1 | SEQ ID NOS: 2793- |
| | | 2811 |
| CHIT1 | Chitinase 1 (chitotriosidase) | SEQ ID NOS: 2812- |
| | | 2815 |
| CHL1 | Cell adhesion molecule L1-like | SEQ ID NOS: 2816- |
| | | 2824 |
| CHN1 | Chimerin 1 | SEQ ID NOS: 2825- |
| | | 2835 |
| CHPF | Chondroitin polymerizing factor | SEQ ID NOS: 2836- |
| | | 2838 |
| CHPF2 | Chondroitin polymerizing factor 2 | SEQ ID NOS: 2839- |
| | | 2842 |
| CHRD | Chordin | SEQ ID NOS: 2843- |
| | | 2848 |
| CHRDL1 | Chordin-like 1 | SEQ ID NOS: 2849- |
| | | 2853 |
| CHRDL2 | Chordin-like 2 | SEQ ID NOS: 2854- |
| | | 2862 |
| CHRNA2 | Cholinergic receptor, nicotinic, alpha 2 | SEQ ID NOS: 2863- |
| | (neuronal) | 2871 |
| CHRNA5 | Cholinergic receptor, nicotinic, alpha 5 | SEQ ID NOS: 2872- |
| | (neuronal) | 2875 |
| CHRNB1 | Cholinergic receptor, nicotinic, beta 1 | SEQ ID NOS: 2876- |
| | (muscle) | 2881 |
| CHRND | Cholinergic receptor, nicotinic, delta | SEQ ID NOS: 2882- |
| | (muscle) | 2887 |
| CHST1 | Carbohydrate (keratan sulfate Gal-6) | SEQ ID NO: 2888 |
| | sulfotransferase 1 | |
| CHST10 | Carbohydrate sulfotransferase 10 | SEQ ID NOS: 2889- |
| | | 2896 |
| CHST11 | Carbohydrate (chondroitin 4) | SEQ ID NOS: 2897- |
| | sulfotransferase 11 | 2901 |
| CHST13 | Carbohydrate (chondroitin 4) | SEQ ID NOS: 2902- |
| | sulfotransferase 13 | 2903 |
| CHST4 | Carbohydrate (N-acetylglucosamine 6-O) | SEQ ID NOS: 2904- |
| | sulfotransferase 4 | 2905 |
| CHST5 | Carbohydrate (N-acetylglucosamine 6-O) | SEQ ID NOS: 2906- |
| | sulfotransferase 5 | 2907 |
| CHST6 | Carbohydrate (N-acetylglucosamine 6-O) | SEQ ID NOS: 2908- |
| | sulfotransferase 6 | 2909 |
| CHST7 | Carbohydrate (N-acetylglucosamine 6-O) | SEQ ID NO: 2910 |
| | sulfotransferase 7 | |
| CHST8 | Carbohydrate (N-acetylgalactosamine 4-O) | SEQ ID NOS: 2911- |
| | sulfotransferase 8 | 2914 |
| CHSY1 | Chondroitin sulfate synthase 1 | SEQ ID NOS: 2915- |
| | | 2916 |
| CHSY3 | Chondroitin sulfate synthase 3 | SEQ ID NO: 2917 |
| CHTF8 | Chromosome transmission fidelity factor 8 | SEQ ID NOS: 2918- |
| | | 2928 |
| CILP | Cartilage intermediate layer protein, | SEQ ID NO: 2929 |
| | nucleotide pyrophosphohydrolase | |
| CILP2 | Cartilage intermediate layer protein 2 | SEQ ID NOS: 2930- |
| | | 2931 |
| CIRH1A | Cirrhosis, autosomal recessive 1A (cirhin) | SEQ ID NOS: 13974- |
| | | 13983 |
| CKLF | Chemokine-like factor | SEQ ID NOS: 2932- |
| | | 2937 |
| CKMT1A | Creatine kinase, mitochondrial 1A | SEQ ID NOS: 2938- |
| | | 2943 |
| CKMT1B | Creatine kinase, mitochondrial 1B | SEQ ID NOS: 2944- |
| | | 2953 |
| CLCA1 | Chloride channel accessory 1 | SEQ ID NOS: 2954- |
| | | 2955 |
| CLCF1 | Cardiotrophin-like cytokine factor 1 | SEQ ID NOS: 2956- |
| | | 2957 |
| CLDN15 | Claudin 15 | SEQ ID NOS: 2958- |
| | | 2963 |
| CLDN7 | Claudin 7 | SEQ ID NOS: 2964- |
| | | 2970 |
| CLDND1 | Claudin domain containing 1 | SEQ ID NOS: 2971- |
| | | 2996 |
| CLEC11A | C-type lectin domain family 11, member A | SEQ ID NOS: 2997- |
| | | 2999 |
| CLEC16A | C-type lectin domain family 16, member A | SEQ ID NOS: 3000- |
| | | 3005 |
| CLEC18A | C-type lectin domain family 18, member A | SEQ ID NOS: 3006- |
| | | 3011 |
| CLEC18B | C-type lectin domain family 18, member B | SEQ ID NOS: 3012- |
| | | 3015 |
| CLEC18C | C-type lectin domain family 18, member C | SEQ ID NOS: 3016- |
| | | 3022 |
| CLEC19A | C-type lectin domain family 19, member A | SEQ ID NOS: 3023- |
| | | 3026 |
| CLEC2B | C-type lectin domain family 2, member B | SEQ ID NOS: 3027- |
| | | 3028 |
| CLEC3A | C-type lectin domain family 3, member A | SEQ ID NOS: 3029- |
| | | 3030 |
| CLEC3B | C-type lectin domain family 3, member B | SEQ ID NOS: 3031- |
| | | 3032 |
| CLGN | Calmegin | SEQ ID NOS: 3033- |
| | | 3035 |
| CLN5 | Ceroid-lipofuscinosis, neuronal 5 | SEQ ID NOS: 3036- |
| | | 3047 |
| CLPS | Colipase, pancreatic | SEQ ID NOS: 3048- |
| | | 3050 |
| CLPSL1 | Colipase-like 1 | SEQ ID NOS: 3051- |
| | | 3052 |
| CLPSL2 | Colipase-like 2 | SEQ ID NOS: 3053- |
| | | 3054 |
| CLPX | Caseinolytic mitochondrial matrix peptidase | SEQ ID NOS: 3055- |
| | chaperone subunit | 3057 |
| CLSTN3 | Calsyntenin 3 | SEQ ID NOS: 3058- |
| | | 3064 |
| CLU | Clusterin | SEQ ID NOS: 3065- |
| | | 3078 |
| CLUL1 | Clusterin-like 1 (retinal) | SEQ ID NOS: 3079- |
| | | 3086 |
| CMA1 | Chymase 1, mast cell | SEQ ID NOS: 3087- |
| | | 3088 |
| CMPK1 | Cytidine monophosphate (UMP-CMP) | SEQ ID NOS: 3089- |
| | kinase 1, cytosolic | 3092 |
| CNBD1 | Cyclic nucleotide binding domain | SEQ ID NOS: 3093- |
| | containing 1 | 3096 |
| CNDP1 | Carnosine dipeptidase 1 (metallopeptidase | SEQ ID NOS: 3097- |
| | M20 family) | 3099 |
| CNPY2 | Canopy FGF signaling regulator 2 | SEQ ID NOS: 3107- |
| | | 3111 |
| CNPY3 | Canopy FGF signaling regulator 3 | SEQ ID NOS: 3112- |
| | | 3113 |
| CNPY4 | Canopy FGF signaling regulator 4 | SEQ ID NOS: 3114- |
| | | 3116 |
| CNTFR | Ciliary neurotrophic factor receptor | SEQ ID NOS: 3117- |
| | | 3120 |
| CNTN1 | Contactin 1 | SEQ ID NOS: 3121- |
| | | 3130 |
| CNTN2 | Contactin 2 (axonal) | SEQ ID NOS: 3131- |
| | | 3142 |
| CNTN3 | Contactin 3 (plasmacytoma associated) | SEQ ID NO: 3143 |
| CNTN4 | Contactin 4 | SEQ ID NOS: 3144- |
| | | 3152 |
| CNTN5 | Contactin 5 | SEQ ID NOS: 3153- |
| | | 3158 |
| CNTNAP2 | Contactin associated protein-like 2 | SEQ ID NOS: 3159- |
| | | 3162 |
| CNTNAP3 | Contactin associated protein-like 3 | SEQ ID NOS: 3163- |
| | | 3167 |
| CNTNAP3B | Contactin associated protein-like 3B | SEQ ID NOS: 3168- |
| | | 3176 |
| COASY | CoA synthase | SEQ ID NOS: 3177- |
| | | 3186 |
| COCH | Cochlin | SEQ ID NOS: 3187- |
| | | 3198 |
| COG3 | Component of oligomeric golgi complex 3 | SEQ ID NOS: 3199- |
| | | 3202 |
| COL10A1 | Collagen, type X, alpha 1 | SEQ ID NOS: 3203- |
| | | 3206 |
| COL11A1 | Collagen, type XI, alpha 1 | SEQ ID NOS: 3207- |
| | | 3217 |
| COL11A2 | Collagen, type XI, alpha 2 | SEQ ID NOS: 3218- |
| | | 3222 |
| COL12A1 | Collagen, type XII, alpha 1 | SEQ ID NOS: 3223- |
| | | 3230 |
| COL14A1 | Collagen, type XIV, alpha 1 | SEQ ID NOS: 3231- |
| | | 3238 |
| COL15A1 | Collagen, type XV, alpha 1 | SEQ ID NOS: 3239- |
| | | 3240 |
| COL16A1 | Collagen, type XVI, alpha 1 | SEQ ID NOS: 3241- |
| | | 3245 |
| COL18A1 | Collagen, type XVIII, alpha 1 | SEQ ID NOS: 3246- |
| | | 3250 |
| COL19A1 | Collagen, type XIX, alpha 1 | SEQ ID NOS: 3251- |
| | | 3253 |
| COL1A1 | Collagen, type I, alpha 1 | SEQ ID NOS: 3254- |
| | | 3255 |
| COL1A2 | Collagen, type I, alpha 2 | SEQ ID NOS: 3256- |
| | | 3257 |
| COL20A1 | Collagen, type XX, alpha 1 | SEQ ID NOS: 3258- |
| | | 3261 |
| COL21A1 | Collagen, type XXI, alpha 1 | SEQ ID NOS: 3262- |
| | | 3267 |
| COL22A1 | Collagen, type XXII, alpha 1 | SEQ ID NOS: 3268- |
| | | 3270 |
| COL24A1 | Collagen, type XXIV, alpha 1 | SEQ ID NOS: 3271- |
| | | 3274 |
| COL26A1 | Collagen, type XXVI, alpha 1 | SEQ ID NOS: 3275- |
| | | 3276 |
| COL27A1 | Collagen, type XXVII, alpha 1 | SEQ ID NOS: 3277- |
| | | 3279 |
| COL28A1 | Collagen, type XXVIII, alpha 1 | SEQ ID NOS: 3280- |
| | | 3284 |
| COL2A1 | Collagen, type II, alpha 1 | SEQ ID NOS: 3285- |
| | | 3286 |
| COL3A1 | Collagen, type III, alpha 1 | SEQ ID NOS: 3287- |
| | | 3289 |
| COL4A1 | Collagen, type IV, alpha 1 | SEQ ID NOS: 3290- |
| | | 3292 |
| COL4A2 | Collagen, type IV, alpha 2 | SEQ ID NOS: 3293- |
| | | 3295 |
| COL4A3 | Collagen, type IV, alpha 3 (Goodpasture | SEQ ID NOS: 3296- |
| | antigen) | 3299 |
| COL4A4 | Collagen, type IV, alpha 4 | SEQ ID NOS: 3300- |
| | | 3301 |
| COL4A5 | Collagen, type IV, alpha 5 | SEQ ID NOS: 3302- |
| | | 3308 |
| COL4A6 | Collagen, type IV, alpha 6 | SEQ ID NOS: 3309- |
| | | 3314 |
| COL5A1 | Collagen, type V, alpha 1 | SEQ ID NOS: 3315- |
| | | 3317 |
| COL5A2 | Collagen, type V, alpha 2 | SEQ ID NOS: 3318- |
| | | 3319 |
| COL5A3 | Collagen, type V, alpha 3 | SEQ ID NO: 3320 |
| COL6A1 | Collagen, type VI, alpha 1 | SEQ ID NOS: 3321- |
| | | 3322 |
| COL6A2 | Collagen, type VI, alpha 2 | SEQ ID NOS: 3323- |
| | | 3328 |
| COL6A3 | Collagen, type VI, alpha 3 | SEQ ID NOS: 3329- |
| | | 3337 |
| COL6A5 | Collagen, type VI, alpha 5 | SEQ ID NOS: 3338- |
| | | 3342 |
| COL6A6 | Collagen, type VI, alpha 6 | SEQ ID NOS: 3343- |
| | | 3345 |
| COL7A1 | Collagen, type VII, alpha 1 | SEQ ID NOS: 3346- |
| | | 3347 |
| COL8A1 | Collagen, type VIII, alpha 1 | SEQ ID NOS: 3348- |
| | | 3351 |
| COL8A2 | Collagen, type VIII, alpha 2 | SEQ ID NOS: 3352- |
| | | 3354 |
| COL9A1 | Collagen, type IX, alpha 1 | SEQ ID NOS: 3355- |
| | | 3358 |
| COL9A2 | Collagen, type IX, alpha 2 | SEQ ID NOS: 3359- |
| | | 3362 |
| COL9A3 | Collagen, type IX, alpha 3 | SEQ ID NOS: 3363- |
| | | 3364 |
| COLEC10 | Collectin sub-family member 10 (C-type | SEQ ID NO: 3365 |
| | lectin) | |
| COLEC11 | Collectin sub-family member 11 | SEQ ID NOS: 3366- |
| | | 3375 |
| COLGALT1 | Collagen beta(1-O)galactosyltransferase 1 | SEQ ID NOS: 3376- |
| | | 3378 |
| COLGALT2 | Collagen beta(1-O)galactosyltransferase 2 | SEQ ID NOS: 3379- |
| | | 3381 |
| COLQ | Collagen-like tail subunit (single strand of | SEQ ID NOS: 3382- |
| | homotrimer) of asymmetric | 3386 |
| | acetylcholinesterase | |
| COMP | Cartilage oligomeric matrix protein | SEQ ID NOS: 3387- |
| | | 3389 |
| C0PS6 | COP9 signalosome subunit 6 | SEQ ID NOS: 3390- |
| | | 3393 |
| COQ6 | Coenzyme Q6 monooxygenase | SEQ ID NOS: 3394- |
| | | 3401 |
| CORT | Cortistatin | SEQ ID NO: 3402 |
| CP | Ceruloplasmin (ferroxidase) | SEQ ID NOS: 3403- |
| | | 3407 |
| CPA1 | Carboxypeptidase A1 (pancreatic) | SEQ ID NOS: 3408- |
| | | 3412 |
| CPA2 | Carboxypeptidase A2 (pancreatic) | SEQ ID NOS: 3413- |
| | | 3414 |
| CPA3 | Carboxypeptidase A3 (mast cell) | SEQ ID NO: 3415 |
| CPA4 | Carboxypeptidase A4 | SEQ ID NOS: 3416- |
| | | 3421 |
| CPA6 | Carboxypeptidase A6 | SEQ ID NOS: 3422- |
| | | 3424 |
| CPAMD8 | C3 and PZP-like, alpha-2-macroglobulin | SEQ ID NOS: 3425- |
| | domain containing 8 | 3430 |
| CPB1 | Carboxypeptidase B1 (tissue) | SEQ ID NOS: 3431- |
| | | 3435 |
| CPB2 | Carboxypeptidase B2 (plasma) | SEQ ID NOS: 3436- |
| | | 3438 |
| CPE | Carboxypeptidase E | SEQ ID NOS: 3439- |
| | | 3443 |
| CPM | Carboxypeptidase M | SEQ ID NOS: 3444- |
| | | 3453 |
| CPN1 | Carboxypeptidase N, polypeptide 1 | SEQ ID NOS: 3454- |
| | | 3455 |
| CPN2 | Carboxypeptidase N, polypeptide 2 | SEQ ID NOS: 3456- |
| | | 3457 |
| CPO | Carboxypeptidase O | SEQ ID NO: 3458 |
| CPQ | Carboxypeptidase Q | SEQ ID NOS: 3459- |
| | | 3464 |
| CPVL | Carboxypeptidase, vitellogenic-like | SEQ ID NOS: 3465- |
| | | 3475 |
| CPXM1 | Carboxypeptidase X (M14 family), member | SEQ ID NO: 3476 |
| | 1 | |
| CPXM2 | Carboxypeptidase X (M14 family), member | SEQ ID NOS: 3477- |
| | 2 | 3478 |
| CPZ | Carboxypeptidase Z | SEQ ID NOS: 3479- |
| | | 3482 |
| CR1L | Complement component (3b/4b) receptor 1- | SEQ ID NOS: 3483- |
| | like | 3484 |
| CRB2 | Crumbs family member 2 | SEQ ID NOS: 3485- |
| | | 3487 |
| CREG1 | Cellular repressor of E1A-stimulated genes | SEQ ID NO: 3488 |
| | 1 | |
| CREG2 | Cellular repressor of E1A-stimulated genes | SEQ ID NO: 3489 |
| | 2 | |
| CRELD1 | Cysteine-rich with EGF-like domains 1 | SEQ ID NOS: 3490- |
| | | 3495 |
| CRELD2 | Cysteine-rich with EGF-like domains 2 | SEQ ID NOS: 3496- |
| | | 3500 |
| CRH | Corticotropin releasing hormone | SEQ ID NO: 3501 |
| CRHBP | Corticotropin releasing hormone binding | SEQ ID NOS: 3502- |
| | protein | 3503 |
| CRHR1 | Corticotropin releasing hormone receptor 1 | SEQ ID NOS: 3504- |
| | | 3515 |
| CRHR2 | Corticotropin releasing hormone receptor 2 | SEQ ID NOS: 3516- |
| | | 3522 |
| CRISP1 | Cysteine-rich secretory protein 1 | SEQ ID NOS: 3523- |
| | | 3526 |
| CRISP2 | Cysteine-rich secretory protein 2 | SEQ ID NOS: 3527- |
| | | 3529 |
| CRISP3 | Cysteine-rich secretory protein 3 | SEQ ID NOS: 3530- |
| | | 3533 |
| CRISPLD2 | Cysteine-rich secretory protein LCCL | SEQ ID NOS: 3534- |
| | domain containing 2 | 3541 |
| CRLF1 | Cytokine receptor-like factor 1 | SEQ ID NOS: 3542- |
| | | 3543 |
| CRP | C-reactive protein, pentraxin-related | SEQ ID NOS: 3544- |
| | | 3548 |
| CRTAC1 | Cartilage acidic protein 1 | SEQ ID NOS: 3549- |
| | | 3553 |
| CRTAP | Cartilage associated protein | SEQ ID NOS: 3554- |
| | | 3555 |
| CRY2 | Cryptochrome circadian clock 2 | SEQ ID NOS: 3556- |
| | | 3559 |
| CSAD | Cysteine sulfinic acid decarboxylase | SEQ ID NOS: 3560- |
| | | 3572 |
| CSF1 | Colony stimulating factor 1 (macrophage) | SEQ ID NOS: 3573- |
| | | 3580 |
| CSF1R | Colony stimulating factor 1 receptor | SEQ ID NOS: 3581- |
| | | 3585 |
| CSF2 | Colony stimulating factor 2 (granulocyte- | SEQ ID NO: 3586 |
| | macrophage) | |
| CSF2RA | Colony stimulating factor 2 receptor, alpha, | SEQ ID NOS: 3587- |
| | low-affinity (granulocyte-macrophage) | 3598 |
| CSF3 | Colony stimulating factor 3 (granulocyte) | SEQ ID NOS: 3599- |
| | | 3605 |
| CSGALNACT1 | Chondroitin sulfate N- | SEQ ID NOS: 3606- |
| | acetylgalactosaminyltransferase 1 | 3614 |
| CSH1 | Chorionic somatomammotropin hormone 1 | SEQ ID NOS: 3615- |
| | (placental lactogen) | 3618 |
| CSH2 | Chorionic somatomammotropin hormone 2 | SEQ ID NOS: 3619- |
| | | 3623 |
| CSHL1 | Chorionic somatomammotropin hormone- | SEQ ID NOS: 3624- |
| | like 1 | 3630 |
| CSN1S1 | Casein alpha s1 | SEQ ID NOS: 3631- |
| | | 3636 |
| CSN2 | Casein beta | SEQ ID NO: 3637 |
| CSN3 | Casein kappa | SEQ ID NO: 3638 |
| CST1 | Cystatin SN | SEQ ID NOS: 3639- |
| | | 3640 |
| CST11 | Cystatin 11 | SEQ ID NOS: 3641- |
| | | 3642 |
| CST2 | Cystatin SA | SEQ ID NO: 3643 |
| CST3 | Cystatin C | SEQ ID NOS: 3644- |
| | | 3646 |
| CST4 | Cystatin S | SEQ ID NO: 3647 |
| CST5 | Cystatin D | SEQ ID NO: 3648 |
| CST6 | Cystatin E/M | SEQ ID NO: 3649 |
| CST7 | Cystatin F (leukocystatin) | SEQ ID NO: 3650 |
| CST8 | Cystatin 8 (cystatin-related epididymal | SEQ ID NOS: 3651- |
| | specific) | 3652 |
| CST9 | Cystatin 9 (testatin) | SEQ ID NO: 3653 |
| CST9L | Cystatin 9-like | SEQ ID NO: 3654 |
| CSTL1 | Cy statin-like 1 | SEQ ID NOS: 3655- |
| | | 3657 |
| CT55 | Cancer/testis antigen 55 | SEQ ID NOS: 3658- |
| | | 3659 |
| CTB-60B18.6 | | SEQ ID NOS: 74-75 |
| CTBS | Chitobiase, di-N-acetyl- | SEQ ID NOS: 3660- |
| | | 3662 |
| CTD- | | SEQ ID NO: 4160 |
| 2313N18.7 | | |
| CTD- | | SEQ ID NOS: 81-84 |
| 2370N5.3 | | |
| CTGF | Connective tissue growth factor | SEQ ID NO: 3663 |
| CTHRC1 | Collagen triple helix repeat containing 1 | SEQ ID NOS: 3664- |
| | | 3667 |
| CTLA4 | Cytotoxic T-lymphocyte-associated protein | SEQ ID NOS: 3668- |
| | 4 | 3671 |
| CTNS | Cystinosin, lysosomal cystine transporter | SEQ ID NOS: 3672- |
| | | 3679 |
| CTRB1 | Chymotrypsinogen B1 | SEQ ID NOS: 3680- |
| | | 3682 |
| CTRB2 | Chymotrypsinogen B2 | SEQ ID NOS: 3683- |
| | | 3686 |
| CTRC | Chymotrypsin C (caldecrin) | SEQ ID NOS: 3687- |
| | | 3688 |
| CTRL | Chymotrypsin-like | SEQ ID NOS: 3689- |
| | | 3691 |
| CTSA | Cathepsin A | SEQ ID NOS: 3692- |
| | | 3700 |
| CTSB | Cathepsin B | SEQ ID NOS: 3701- |
| | | 3725 |
| CTSC | Cathepsin C | SEQ ID NOS: 3726- |
| | | 3730 |
| CTSD | Cathepsin D | SEQ ID NOS: 3731- |
| | | 3741 |
| CTSE | Cathepsin E | SEQ ID NOS: 3742- |
| | | 3743 |
| CTSF | Cathepsin F | SEQ ID NOS: 3744- |
| | | 3747 |
| CTSG | Cathepsin G | SEQ ID NO: 3748 |
| CTSH | Cathepsin H | SEQ ID NOS: 3749- |
| | | 3754 |
| CTSK | Cathepsin K | SEQ ID NOS: 3755- |
| | | 3756 |
| CTSL | Cathepsin L | SEQ ID NOS: 3757- |
| | | 3759 |
| CTSO | Cathepsin O | SEQ ID NO: 3760 |
| CTSS | Cathepsin S | SEQ ID NOS: 3761- |
| | | 3765 |
| CTSV | Cathepsin V | SEQ ID NOS: 3766- |
| | | 3767 |
| CTSW | Cathepsin W | SEQ ID NOS: 3768- |
| | | 3770 |
| CTSZ | Cathepsin Z | SEQ ID NO: 3771 |
| CUBN | Cubilin (intrinsic factor-cobalamin receptor) | SEQ ID NOS: 3772- |
| | | 3775 |
| CUTA | CutA divalent cation tolerance homolog | SEQ ID NOS: 3776- |
| | (E. coli) | 3785 |
| CX3CL1 | Chemokine (C-X3-C motif) ligand 1 | SEQ ID NOS: 3786- |
| | | 3789 |
| CXADR | Coxsackie virus and adenovirus receptor | SEQ ID NOS: 3790- |
| | | 3794 |
| CXCL1 | Chemokine (C-X-C motif) ligand 1 | SEQ ID NO: 3795 |
| | (melanoma growth stimulating activity, | |
| | alpha) | |
| CXCL10 | Chemokine (C-X-C motif) ligand 10 | SEQ ID NO: 3796 |
| CXCL11 | Chemokine (C-X-C motif) ligand 11 | SEQ ID NOS: 3797- |
| | | 3798 |
| CXCL12 | Chemokine (C-X-C motif) ligand 12 | SEQ ID NOS: 3799- |
| | | 3804 |
| CXCL13 | Chemokine (C-X-C motif) ligand 13 | SEQ ID NO: 3805 |
| CXCL14 | Chemokine (C-X-C motif) ligand 14 | SEQ ID NOS: 3806- |
| | | 3807 |
| CXCL17 | Chemokine (C-X-C motif) ligand 17 | SEQ ID NOS: 3808- |
| | | 3809 |
| CXCL2 | Chemokine (C-X-C motif) ligand 2 | SEQ ID NO: 3810 |
| CXCL3 | Chemokine (C-X-C motif) ligand 3 | SEQ ID NO: 3811 |
| CXCL5 | Chemokine (C-X-C motif) ligand 5 | SEQ ID NO: 3812 |
| CXCL6 | Chemokine (C-X-C motif) ligand 6 | SEQ ID NOS: 3813- |
| | | 3814 |
| CXCL8 | Chemokine (C-X-C motif) ligand 8 | SEQ ID NOS: 3815- |
| | | 3816 |
| CXCL9 | Chemokine (C-X-C motif) ligand 9 | SEQ ID NO: 3817 |
| CXorf36 | Chromosome X open reading frame 36 | SEQ ID NOS: 3818- |
| | | 3819 |
| CYB5D2 | Cytochrome b5 domain containing 2 | SEQ ID NOS: 3820- |
| | | 3823 |
| CYHR1 | Cysteine/histidine-rich 1 | SEQ ID NOS: 3824- |
| | | 3831 |
| CYP17A1 | Cytochrome P450, family 17, subfamily A, | SEQ ID NOS: 3832- |
| | polypeptide 1 | 3836 |
| CYP20A1 | Cytochrome P450, family 20, subfamily A, | SEQ ID NOS: 3837- |
| | polypeptide 1 | 3843 |
| CYP21A2 | Cytochrome P450, family 21, subfamily A, | SEQ ID NOS: 3844- |
| | polypeptide 2 | 3851 |
| CYP26B1 | Cytochrome P450, family 26, subfamily B, | SEQ ID NOS: 3852- |
| | polypeptide 1 | 3856 |
| CYP2A6 | Cytochrome P450, family 2, subfamily A, | SEQ ID NOS: 3857- |
| | polypeptide 6 | 3858 |
| CYP2A7 | Cytochrome P450, family 2, subfamily A, | SEQ ID NOS: 3859- |
| | polypeptide 7 | 3861 |
| CYP2B6 | Cytochrome P450, family 2, subfamily B, | SEQ ID NOS: 3862- |
| | polypeptide 6 | 3865 |
| CYP2C18 | Cytochrome P450, family 2, subfamily C, | SEQ ID NOS: 3866- |
| | polypeptide 18 | 3867 |
| CYP2C19 | Cytochrome P450, family 2, subfamily C, | SEQ ID NOS: 3868- |
| | polypeptide 19 | 3869 |
| CYP2C8 | Cytochrome P450, family 2, subfamily C, | SEQ ID NOS: 3870- |
| | polypeptide 8 | 3877 |
| CYP2C9 | Cytochrome P450, family 2, subfamily C, | SEQ ID NOS: 3878- |
| | polypeptide 9 | 3880 |
| CYP2E1 | Cytochrome P450, family 2, subfamily E, | SEQ ID NOS: 3881- |
| | polypeptide 1 | 3886 |
| CYP2F1 | Cytochrome P450, family 2, subfamily F, | SEQ ID NOS: 3887- |
| | polypeptide 1 | 3890 |
| CYP2J2 | Cytochrome P450, family 2, subfamily J, | SEQ ID NO: 3891 |
| | polypeptide 2 | |
| CYP2R1 | Cytochrome P450, family 2, subfamily R, | SEQ ID NOS: 3892- |
| | polypeptide 1 | 3897 |
| CYP2S1 | Cytochrome P450, family 2, subfamily S, | SEQ ID NOS: 3898- |
| | polypeptide 1 | 3903 |
| CYP2W1 | Cytochrome P450, family 2, subfamily W, | SEQ ID NOS: 3904- |
| | polypeptide 1 | 3906 |
| CYP46A1 | Cytochrome P450, family 46, subfamily A, | SEQ ID NOS: 3907- |
| | polypeptide 1 | 3911 |
| CYP4F11 | Cytochrome P450, family 4, subfamily F, | SEQ ID NOS: 3912- |
| | polypeptide 11 | 3916 |
| CYP4F2 | Cytochrome P450, family 4, subfamily F, | SEQ ID NOS: 3917- |
| | polypeptide 2 | 3921 |
| CYR61 | Cysteine-rich, angiogenic inducer, 61 | SEQ ID NO: 3922 |
| CYTL1 | Cytokine-like 1 | SEQ ID NOS: 3923- |
| | | 3925 |
| D2HGDH | D-2-hydroxyglutarate dehydrogenase | SEQ ID NOS: 3926- |
| | | 3934 |
| DAG1 | Dystroglycan 1 (dystrophin-associated | SEQ ID NOS: 3935- |
| | glycoprotein 1) | 3949 |
| DAND5 | DAN domain family member 5, BMP | SEQ ID NOS: 3950- |
| | antagonist | 3951 |
| DAO | D-amino-acid oxidase | SEQ ID NOS: 3952- |
| | | 3957 |
| DAZAP2 | DAZ associated protein 2 | SEQ ID NOS: 3958- |
| | | 3966 |
| DBH | Dopamine beta-hydroxylase (dopamine | SEQ ID NOS: 3967- |
| | beta-monooxygenase) | 3968 |
| DBNL | Drebrin-like | SEQ ID NOS: 3969- |
| | | 3986 |
| DCD | Dermcidin | SEQ ID NOS: 3987- |
| | | 3989 |
| DCN | Decorin | SEQ ID NOS: 3990- |
| | | 4008 |
| DDIAS | DNA damage-induced apoptosis suppressor | SEQ ID NOS: 4009- |
| | | 4018 |
| DDOST | Dolichyl-diphosphooligosaccharide--protein | SEQ ID NOS: 4019- |
| | glycosyltransferase subunit (non-catalytic) | 4022 |
| DDR1 | Discoidin domain receptor tyrosine kinase 1 | SEQ ID NOS: 4023- |
| | | 4068 |
| DDR2 | Discoidin domain receptor tyrosine kinase 2 | SEQ ID NOS: 4069- |
| | | 4074 |
| DDT | D-dopachrome tautomerase | SEQ ID NOS: 4075- |
| | | 4080 |
| DDX17 | DEAD (Asp-Glu-Ala-Asp) box helicase 17 | SEQ ID NOS: 4081- |
| | | 4085 |
| DDX20 | DEAD (Asp-Glu-Ala-Asp) box polypeptide | SEQ ID NOS: 4086- |
| | 20 | 4088 |
| DDX25 | DEAD (Asp-Glu-Ala-Asp) box helicase 25 | SEQ ID NOS: 4089- |
| | | 4095 |
| DDX28 | DEAD (Asp-Glu-Ala-Asp) box polypeptide | SEQ ID NO: 4096 |
| | 28 | |
| DEAF1 | DEAF1 transcription factor | SEQ ID NOS: 4097- |
| | | 4099 |
| DEF8 | Differentially expressed in FDCP 8 | SEQ ID NOS: 4100- |
| | homolog (mouse) | 4119 |
| DEFA1 | Defensin, alpha 1 | SEQ ID NOS: 4120- |
| | | 4121 |
| DEFA1B | Defensin, alpha 1B | SEQ ID NO: 4122 |
| DEFA3 | Defensin, alpha 3, neutrophil-specific | SEQ ID NO: 4123 |
| DEFA4 | Defensin, alpha 4, corticostatin | SEQ ID NO: 4124 |
| DEFA5 | Defensin, alpha 5, Paneth cell-specific | SEQ ID NO: 4125 |
| DEFA6 | Defensin, alpha 6, Paneth cell-specific | SEQ ID NO: 4126 |
| DEFB1 | Defensin, beta 1 | SEQ ID NO: 4127 |
| DEFB103A | Defensin, beta 103A | SEQ ID NO: 4128 |
| DEFB103B | Defensin, beta 103B | SEQ ID NO: 4129 |
| DEFB104A | Defensin, beta 104A | SEQ ID NO: 4130 |
| DEFB104B | Defensin, beta 104B | SEQ ID NO: 4131 |
| DEFB105A | Defensin, beta 105A | SEQ ID NO: 4132 |
| DEFB105B | Defensin, beta 105B | SEQ ID NO: 4133 |
| DEFB106A | Defensin, beta 106A | SEQ ID NO: 4134 |
| DEFB106B | Defensin, beta 106B | SEQ ID NO: 4135 |
| DEFB107A | Defensin, beta 107A | SEQ ID NO: 4136 |
| DEFB107B | Defensin, beta 107B | SEQ ID NO: 4137 |
| DEFB108B | Defensin, beta 108B | SEQ ID NO: 4138 |
| DEFB110 | Defensin, beta 110 | SEQ ID NOS: 4139- |
| | | 4140 |
| DEFB113 | Defensin, beta 113 | SEQ ID NO: 4141 |
| DEFB114 | Defensin, beta 114 | SEQ ID NO: 4142 |
| DEFB115 | Defensin, beta 115 | SEQ ID NO: 4143 |
| DEFB116 | Defensin, beta 116 | SEQ ID NO: 4144 |
| DEFB118 | Defensin, beta 118 | SEQ ID NO: 4145 |
| DEFB119 | Defensin, beta 119 | SEQ ID NOS: 4146- |
| | | 4148 |
| DEFB121 | Defensin, beta 121 | SEQ ID NO: 4149 |
| DEFB123 | Defensin, beta 123 | SEQ ID NO: 4150 |
| DEFB124 | Defensin, beta 124 | SEQ ID NO: 4151 |
| DEFB125 | Defensin, beta 125 | SEQ ID NO: 4152 |
| DEFB126 | Defensin, beta 126 | SEQ ID NO: 4153 |
| DEFB127 | Defensin, beta 127 | SEQ ID NO: 4154 |
| DEFB128 | Defensin, beta 128 | SEQ ID NO: 4155 |
| DEFB129 | Defensin, beta 129 | SEQ ID NO: 4156 |
| DEFB130 | Defensin, beta 130 | SEQ ID NO: 4157 |
| DEFB131 | Defensin, beta 131 | SEQ ID NO: 4159 |
| DEFB132 | Defensin, beta 132 | SEQ ID NO: 4161 |
| DEFB133 | Defensin, beta 133 | SEQ ID NO: 4162 |
| DEFB134 | Defensin, beta 134 | SEQ ID NOS: 4163- |
| | | 4164 |
| DEFB135 | Defensin, beta 135 | SEQ ID NO: 4165 |
| DEFB136 | Defensin, beta 136 | SEQ ID NO: 4166 |
| DEFB4A | Defensin, beta 4A | SEQ ID NO: 4167 |
| DEFB4B | Defensin, beta 4B | SEQ ID NO: 4168 |
| DFNA5 | Deafness, autosomal dominant 5 | SEQ ID NOS: 6271- |
| | | 6279 |
| DFNB31 | Deafness, autosomal recessive 31 | SEQ ID NOS: 14251- |
| | | 14254 |
| DGCR2 | DiGeorge syndrome critical region gene 2 | SEQ ID NOS: 4171- |
| | | 4174 |
| DHH | Desert hedgehog | SEQ ID NO: 4175 |
| DHRS4 | Dehydrogenase/reductase (SDR family) | SEQ ID NOS: 4176- |
| | member 4 | 4183 |
| DHRS4L2 | Dehydrogenase/reductase (SDR family) | SEQ ID NOS: 4184- |
| | member 4 like 2 | 4193 |
| DHRS7 | Dehydrogenase/reductase (SDR family) | SEQ ID NOS: 4194- |
| | member 7 | 4201 |
| DHRS7C | Dehydrogenase/reductase (SDR family) | SEQ ID NOS: 4202- |
| | member 7C | 4204 |
| DHRS9 | Dehydrogenase/reductase (SDR family) | SEQ ID NOS: 4205- |
| | member 9 | 4212 |
| DHRSX | Dehydrogenase/reductase (SDR family) X- | SEQ ID NOS: 4213- |
| | linked | 4217 |
| DHX29 | DEAH (Asp-Glu-Ala-His) box polypeptide | SEQ ID NOS: 4218- |
| | 29 | 4220 |
| DHX30 | DEAH (Asp-Glu-Ala-His) box helicase 30 | SEQ ID NOS: 4221- |
| | | 4228 |
| DHX8 | DEAH (Asp-Glu-Ala-His) box polypeptide | SEQ ID NOS: 4229- |
| | 8 | 4233 |
| DIO2 | Deiodinase, iodothyronine, type II | SEQ ID NOS: 4234- |
| | | 4243 |
| DIXDC1 | DIX domain containing 1 | SEQ ID NOS: 4244- |
| | | 4247 |
| DKK1 | Dickkopf WNT signaling pathway inhibitor | SEQ ID NO: 4248 |
| | 1 | |
| DKK2 | Dickkopf WNT signaling pathway inhibitor | SEQ ID NOS: 4249- |
| | 2 | 4251 |
| DKK3 | Dickkopf WNT signaling pathway inhibitor | SEQ ID NOS: 4252- |
| | 3 | 4257 |
| DKK4 | Dickkopf WNT signaling pathway inhibitor | SEQ ID NO: 4258 |
| | 4 | |
| DKKL1 | Dickkopf-like 1 | SEQ ID NOS: 4259- |
| | | 4264 |
| DLG4 | Discs, large homolog 4 (Drosophila) | SEQ ID NOS: 4265- |
| | | 4273 |
| DLK1 | Delta-like 1 homolog (Drosophila) | SEQ ID NOS: 4274- |
| | | 4277 |
| DLL1 | Delta-like 1 (Drosophila) | SEQ ID NOS: 4278- |
| | | 4279 |
| DLL3 | Delta-like 3 (Drosophila) | SEQ ID NOS: 4280- |
| | | 4282 |
| DMBT1 | Deleted in malignant brain tumors 1 | SEQ ID NOS: 4283- |
| | | 4289 |
| DMKN | Dermokine | SEQ ID NOS: 4290- |
| | | 4336 |
| DMP1 | Dentin matrix acidic phosphoprotein 1 | SEQ ID NOS: 4337- |
| | | 4338 |
| DMRTA2 | DMRT-like family A2 | SEQ ID NOS: 4339- |
| | | 4340 |
| DNAAF5 | Dynein, axonemal, assembly factor 5 | SEQ ID NOS: 4341- |
| | | 4344 |
| DNAH14 | Dynein, axonemal, heavy chain 14 | SEQ ID NOS: 4345- |
| | | 4359 |
| DNAJB11 | DnaJ (Hsp40) homolog, subfamily B, | SEQ ID NOS: 4360- |
| | member 11 | 4361 |
| DNAJB9 | DnaJ (Hsp40) homolog, subfamily B, | SEQ ID NO: 4362 |
| | member 9 | |
| DNAJC25- | DNAJC25-GNG10 readthrough | SEQ ID NO: 4363 |
| GNG10 | | |
| DNAJC3 | DnaJ (Hsp40) homolog, subfamily C, | SEQ ID NOS: 4364- |
| | member 3 | 4365 |
| DNASE1 | Deoxyribonuclease I | SEQ ID NOS: 4366- |
| | | 4376 |
| DNASE1L1 | Deoxyribonuclease I-like 1 | SEQ ID NOS: 4377- |
| | | 4387 |
| DNASE1L2 | Deoxyribonuclease I-like 2 | SEQ ID NOS: 4388- |
| | | 4393 |
| DNASE1L3 | Deoxyribonuclease I-like 3 | SEQ ID NOS: 4394- |
| | | 4399 |
| DNASE2 | Deoxyribonuclease II, lysosomal | SEQ ID NOS: 4400- |
| | | 4401 |
| DNASE2B | Deoxyribonuclease II beta | SEQ ID NOS: 4402- |
| | | 4403 |
| DPEP1 | Dipeptidase 1 (renal) | SEQ ID NOS: 4404- |
| | | 4408 |
| DPEP2 | Dipeptidase 2 | SEQ ID NOS: 4409- |
| | | 4415 |
| DPEP3 | Dipeptidase 3 | SEQ ID NO: 4416 |
| DPF3 | D4, zinc and double PHD fingers, family 3 | SEQ ID NOS: 4417- |
| | | 4423 |
| DPP4 | Dipeptidyl-peptidase 4 | SEQ ID NOS: 4424- |
| | | 4428 |
| DPP7 | Dipeptidyl-peptidase 7 | SEQ ID NOS: 4429- |
| | | 4434 |
| DPT | Dermatopontin | SEQ ID NO: 4435 |
| DRAXIN | Dorsal inhibitory axon guidance protein | SEQ ID NO: 4436 |
| DSE | Dermatan sulfate epimerase | SEQ ID NOS: 4437- |
| | | 4445 |
| DSG2 | Desmoglein 2 | SEQ ID NOS: 4446- |
| | | 4447 |
| DSPP | Dentin sialophosphoprotein | SEQ ID NOS: 4448- |
| | | 4449 |
| DST | Dystonin | SEQ ID NOS: 4450- |
| | | 4468 |
| DUOX1 | Dual oxidase 1 | SEQ ID NOS: 4469- |
| | | 4473 |
| DYNLT3 | Dynein, light chain, Tctex-type 3 | SEQ ID NOS: 4474- |
| | | 4476 |
| E2F5 | E2F transcription factor 5, p130-binding | SEQ ID NOS: 4477- |
| | | 4483 |
| EBAG9 | Estrogen receptor binding site associated, | SEQ ID NOS: 4484- |
| | antigen, 9 | 4492 |
| EBI3 | Epstein-Barr virus induced 3 | SEQ ID NO: 4493 |
| ECHDC1 | Ethylmalonyl-CoA decarboxylase 1 | SEQ ID NOS: 4494- |
| | | 4512 |
| ECM1 | Extracellular matrix protein 1 | SEQ ID NOS: 4513- |
| | | 4515 |
| ECM2 | Extracellular matrix protein 2, female organ | SEQ ID NOS: 4516- |
| | and adipocyte specific | 4519 |
| ECSIT | ECSIT signalling integrator | SEQ ID NOS: 4520- |
| | | 4531 |
| EDDM3A | Epididymal protein 3A | SEQ ID NO: 4532 |
| EDDM3B | Epididymal protein 3B | SEQ ID NO: 4533 |
| EDEM2 | ER degradation enhancer, mannosidase | SEQ ID NOS: 4534- |
| | alpha-like 2 | 4535 |
| EDEM3 | ER degradation enhancer, mannosidase | SEQ ID NOS: 4536- |
| | alpha-like 3 | 4538 |
| EDIL3 | EGF-like repeats and discoidin I-like | SEQ ID NOS: 4539- |
| | domains 3 | 4540 |
| EDN1 | Endothelin 1 | SEQ ID NO: 4541 |
| EDN2 | Endothelin 2 | SEQ ID NO: 4542 |
| EDN3 | Endothelin 3 | SEQ ID NOS: 4543- |
| | | 4548 |
| EDNRB | Endothelin receptor type B | SEQ ID NOS: 4549- |
| | | 4557 |
| EFEMP1 | EGF containing fibulin-like extracellular | SEQ ID NOS: 4558- |
| | matrix protein 1 | 4568 |
| EFEMP2 | EGF containing fibulin-like extracellular | SEQ ID NOS: 4569- |
| | matrix protein 2 | 4580 |
| EFNA1 | Ephrin-A1 | SEQ ID NOS: 4581- |
| | | 4582 |
| EFNA2 | Ephrin-A2 | SEQ ID NO: 4583 |
| EFNA4 | Ephrin-A4 | SEQ ID NOS: 4584- |
| | | 4586 |
| EGFL6 | EGF-like-domain, multiple 6 | SEQ ID NOS: 4587- |
| | | 4588 |
| EGFL7 | EGF-like-domain, multiple 7 | SEQ ID NOS: 4589- |
| | | 4593 |
| EGFL8 | EGF-like-domain, multiple 8 | SEQ ID NOS: 4594- |
| | | 4596 |
| EGFLAM | EGF-like, fibronectin type III and laminin G | SEQ ID NOS: 4597- |
| | domains | 4605 |
| EGFR | Epidermal growth factor receptor | SEQ ID NOS: 4606- |
| | | 4613 |
| EHBP1 | EH domain binding protein 1 | SEQ ID NOS: 4614- |
| | | 4625 |
| EHF | Ets homologous factor | SEQ ID NOS: 4626- |
| | | 4635 |
| EHMT1 | Euchromatic histone-lysine N- | SEQ ID NOS: 4636- |
| | methyltransferase 1 | 4661 |
| EHMT2 | Euchromatic histone-lysine N- | SEQ ID NOS: 4662- |
| | methyltransferase 2 | 4666 |
| EIF2AK1 | Eukaryotic translation initiation factor 2- | SEQ ID NOS: 4667- |
| | alpha kinase 1 | 4670 |
| ELANE | Elastase, neutrophil expressed | SEQ ID NOS: 4671- |
| | | 4672 |
| ELN | Elastin | SEQ ID NOS: 4673- |
| | | 4695 |
| ELP2 | Elongator acetyltransferase complex subunit | SEQ ID NOS: 4696- |
| | 2 | 4708 |
| ELSPBP1 | Epididymal sperm binding protein 1 | SEQ ID NOS: 4709- |
| | | 4714 |
| EMC1 | ER membrane protein complex subunit 1 | SEQ ID NOS: 4715- |
| | | 4721 |
| EMC10 | ER membrane protein complex subunit 10 | SEQ ID NOS: 4722- |
| | | 4728 |
| EMC9 | ER membrane protein complex subunit 9 | SEQ ID NOS: 4729- |
| | | 4732 |
| EMCN | Endomucin | SEQ ID NOS: 4733- |
| | | 4737 |
| EMID1 | EMI domain containing 1 | SEQ ID NOS: 4738- |
| | | 4744 |
| EMILIN1 | Elastin microfibril interfacer 1 | SEQ ID NOS: 4745- |
| | | 4746 |
| EMILIN2 | Elastin microfibril interfacer 2 | SEQ ID NO: 4747 |
| EMILIN3 | Elastin microfibril interfacer 3 | SEQ ID NO: 4748 |
| ENAM | Enamelin | SEQ ID NO: 4749 |
| ENDOG | Endonuclease G | SEQ ID NO: 4750 |
| ENDOU | Endonuclease, polyU-specific | SEQ ID NOS: 4751- |
| | | 4753 |
| ENHO | Energy homeostasis associated | SEQ ID NO: 4754 |
| ENO4 | Enolase family member 4 | SEQ ID NOS: 4755- |
| | | 4759 |
| ENPP6 | Ectonucleotide | SEQ ID NOS: 4760- |
| | pyrophosphatase/phosphodiesterase 6 | 4761 |
| ENPP7 | Ectonucleotide | SEQ ID NOS: 4762- |
| | pyrophosphatase/phosphodiesterase 7 | 4763 |
| ENTPD5 | Ectonucleoside triphosphate | SEQ ID NOS: 4764- |
| | diphosphohydrolase 5 | 4768 |
| ENTPD8 | Ectonucleoside triphosphate | SEQ ID NOS: 4769- |
| | diphosphohydrolase 8 | 4772 |
| EOGT | EGF domain-specific O-linked N- | SEQ ID NOS: 4773- |
| | acetylglucosamine (GlcNAc) transferase | 4780 |
| EPCAM | Epithelial cell adhesion molecule | SEQ ID NOS: 4781- |
| | | 4784 |
| EPDR1 | Ependymin related 1 | SEQ ID NOS: 4785- |
| | | 4788 |
| EPGN | Epithelial mitogen | SEQ ID NOS: 4789- |
| | | 4797 |
| EPHA10 | EPH receptor A10 | SEQ ID NOS: 4798- |
| | | 4805 |
| EPHA3 | EPH receptor A3 | SEQ ID NOS: 4806- |
| | | 4808 |
| EPHA4 | EPH receptor A4 | SEQ ID NOS: 4809- |
| | | 4818 |
| EPHA7 | EPH receptor A7 | SEQ ID NOS: 4819- |
| | | 4820 |
| EPHA8 | EPH receptor A8 | SEQ ID NOS: 4821- |
| | | 4822 |
| EPHB2 | EPH receptor B2 | SEQ ID NOS: 4823- |
| | | 4827 |
| EPHB4 | EPH receptor B4 | SEQ ID NOS: 4828- |
| | | 4830 |
| EPHX3 | Epoxide hydrolase 3 | SEQ ID NOS: 4831- |
| | | 4834 |
| EPO | Erythropoietin | SEQ ID NO: 4835 |
| EPPIN | Epididymal peptidase inhibitor | SEQ ID NOS: 4836- |
| | | 4838 |
| EPPIN- | EPPIN-WFDC6 readthrough | SEQ ID NO: 4839 |
| WFDC6 | | |
| EPS15 | Epidermal growth factor receptor pathway | SEQ ID NOS: 4840- |
| | substrate 15 | 4842 |
| EPS8L1 | EPS8-like 1 | SEQ ID NOS: 4843- |
| | | 4848 |
| EPX | Eosinophil peroxidase | SEQ ID NO: 4849 |
| EPYC | Epiphycan | SEQ ID NOS: 4850- |
| | | 4851 |
| EQTN | Equatorin, sperm acrosome associated | SEQ ID NOS: 4852- |
| | | 4854 |
| ERAP1 | Endoplasmic reticulum aminopeptidase 1 | SEQ ID NOS: 4855- |
| | | 4859 |
| ERAP2 | Endoplasmic reticulum aminopeptidase 2 | SEQ ID NOS: 4860- |
| | | 4867 |
| ERBB3 | Erb-b2 receptor tyrosine kinase 3 | SEQ ID NOS: 4868- |
| | | 4881 |
| ERLIN1 | ER lipid raft associated 1 | SEQ ID NOS: 4885- |
| | | 4887 |
| ERLIN2 | ER lipid raft associated 2 | SEQ ID NOS: 4888- |
| | | 4896 |
| ERN1 | Endoplasmic reticulum to nucleus signaling | SEQ ID NOS: 4897- |
| | 1 | 4898 |
| ERN2 | Endoplasmic reticulum to nucleus signaling | SEQ ID NOS: 4899- |
| | 2 | 4903 |
| ERO1A | Endoplasmic reticulum oxidoreductase | SEQ ID NOS: 4904- |
| | alpha | 4910 |
| ERO1B | Endoplasmic reticulum oxidoreductase beta | SEQ ID NOS: 4911- |
| | | 4913 |
| ERP27 | Endoplasmic reticulum protein 27 | SEQ ID NOS: 4914- |
| | | 4915 |
| ERP29 | Endoplasmic reticulum protein 29 | SEQ ID NOS: 4916- |
| | | 4919 |
| ERP44 | Endoplasmic reticulum protein 44 | SEQ ID NO: 4920 |
| ERV3-1 | Endogenous retrovirus group 3, member 1 | SEQ ID NO: 4921 |
| ESM1 | Endothelial cell-specific molecule 1 | SEQ ID NOS: 4922- |
| | | 4924 |
| ESRP1 | Epithelial splicing regulatory protein 1 | SEQ ID NOS: 4925- |
| | | 4933 |
| EXOG | Endo/exonuclease (5′-3′), endonuclease G- | SEQ ID NOS: 4934- |
| | like | 4947 |
| EXTL1 | Exostosin-like glycosyltransferase 1 | SEQ ID NO: 4948 |
| EXTL2 | Exostosin-like glycosyltransferase 2 | SEQ ID NOS: 4949- |
| | | 4953 |
| F10 | Coagulation factor X | SEQ ID NOS: 4954- |
| | | 4957 |
| F11 | Coagulation factor XI | SEQ ID NOS: 4958- |
| | | 4962 |
| F12 | Coagulation factor XII (Hageman factor) | SEQ ID NO: 4963 |
| F13B | Coagulation factor XIII, B polypeptide | SEQ ID NO: 4964 |
| F2 | Coagulation factor II (thrombin) | SEQ ID NOS: 4965- |
| | | 4967 |
| F2R | Coagulation factor II (thrombin) receptor | SEQ ID NOS: 4968- |
| | | 4969 |
| F2RL3 | Coagulation factor II (thrombin) receptor- | SEQ ID NOS: 4970- |
| | like 3 | 4971 |
| F5 | Coagulation factor V (proaccelerin, labile | SEQ ID NOS: 4972- |
| | factor) | 4973 |
| F7 | Coagulation factor VII (serum prothrombin | SEQ ID NOS: 4974- |
| | conversion accelerator) | 4977 |
| F8 | Coagulation factor VIII, procoagulant | SEQ ID NOS: 4978- |
| | component | 4983 |
| F9 | Coagulation factor IX | SEQ ID NOS: 4984- |
| | | 4985 |
| FABP6 | Fatty acid binding protein 6, ileal | SEQ ID NOS: 4986- |
| | | 4988 |
| FAM107B | Family with sequence similarity 107, | SEQ ID NOS: 4989- |
| | member B | 5010 |
| FAM131A | Family with sequence similarity 131, | SEQ ID NOS: 5011- |
| | member A | 5019 |
| FAM132A | Family with sequence similarity 132, | SEQ ID NO: 1795 |
| | member A | |
| FAM132B | Family with sequence similarity 132, | SEQ ID NOS: 4882- |
| | member B | 4884 |
| FAM150A | Family with sequence similarity 150, | SEQ ID NOS: 737-738 |
| | member A | |
| FAM150B | Family with sequence similarity 150, | SEQ ID NOS: 739-745 |
| | member B | |
| FAM171A1 | Family with sequence similarity 171, | SEQ ID NOS: 5020- |
| | member A1 | 5021 |
| FAM171B | Family with sequence similarity 171, | SEQ ID NOS: 5022- |
| | member B | 5023 |
| FAM172A | Family with sequence similarity 172, | SEQ ID NOS: 5024- |
| | member A | 5028 |
| FAM175A | Family with sequence similarity 175, | SEQ ID NOS: 64-71 |
| | member A | |
| FAM177A1 | Family with sequence similarity 177, | SEQ ID NOS: 5029- |
| | member A1 | 5038 |
| FAM179B | Family with sequence similarity 179, | SEQ ID NOS: 13628- |
| | member B | 13633 |
| FAM180A | Family with sequence similarity 180, | SEQ ID NOS: 5039- |
| | member A | 5041 |
| FAM189A1 | Family with sequence similarity 189, | SEQ ID NOS: 5042- |
| | member A1 | 5043 |
| FAM198A | Family with sequence similarity 198, | SEQ ID NOS: 5044- |
| | member A | 5046 |
| FAM19A1 | Family with sequence similarity 19 | SEQ ID NOS: 5047- |
| | (chemokine (C-C motif)-like), member A1 | 5049 |
| FAM19A2 | Family with sequence similarity 19 | SEQ ID NOS: 5050- |
| | (chemokine (C-C motif)-like), member A2 | 5057 |
| FAM19A3 | Family with sequence similarity 19 | SEQ ID NOS: 5058- |
| | (chemokine (C-C motif)-like), member A3 | 5059 |
| FAM19A4 | Family with sequence similarity 19 | SEQ ID NOS: 5060- |
| | (chemokine (C-C motif)-like), member A4 | 5062 |
| FAM19A5 | Family with sequence similarity 19 | SEQ ID NOS: 5063- |
| | (chemokine (C-C motif)-like), member A5 | 5066 |
| FAM20A | Family with sequence similarity 20, | SEQ ID NOS: 5067- |
| | member A | 5070 |
| FAM20C | Family with sequence similarity 20, | SEQ ID NO: 5071 |
| | member C | |
| FAM213A | Family with sequence similarity 213, | SEQ ID NOS: 5072- |
| | member A | 5077 |
| FAM26D | Family with sequence similarity 26, | SEQ ID NOS: 2006- |
| | member D | 2010 |
| FAM46B | Family with sequence similarity 46, | SEQ ID NO: 5078 |
| | member B | |
| FAM57A | Family with sequence similarity 57, | SEQ ID NOS: 5079- |
| | member A | 5084 |
| FAM78A | Family with sequence similarity 78, | SEQ ID NOS: 5085- |
| | member A | 5087 |
| FAM96A | Family with sequence similarity 96, | SEQ ID NOS: 5088- |
| | member A | 5092 |
| FAM9B | Family with sequence similarity 9, member | SEQ ID NOS: 5093- |
| | B | 5096 |
| FAP | Fibroblast activation protein, alpha | SEQ ID NOS: 5097- |
| | | 5103 |
| FAS | Fas cell surface death receptor | SEQ ID NOS: 5104- |
| | | 5113 |
| FAT1 | FAT atypical cadherin 1 | SEQ ID NOS: 5114- |
| | | 5120 |
| FBLN1 | Fibulin 1 | SEQ ID NOS: 5121- |
| | | 5133 |
| FBLN2 | Fibulin 2 | SEQ ID NOS: 5134- |
| | | 5139 |
| FBLN5 | Fibulin 5 | SEQ ID NOS: 5140- |
| | | 5145 |
| FBLN7 | Fibulin 7 | SEQ ID NOS: 5146- |
| | | 5151 |
| FBN1 | Fibrillin 1 | SEQ ID NOS: 5152- |
| | | 5155 |
| FBN2 | Fibrillin 2 | SEQ ID NOS: 5156- |
| | | 5161 |
| FBN3 | Fibrillin 3 | SEQ ID NOS: 5162- |
| | | 5166 |
| FBXW7 | F-box and WD repeat domain containing 7, | SEQ ID NOS: 5167- |
| | E3 ubiquitin protein ligase | 5177 |
| FCAR | Fc fragment of IgA receptor | SEQ ID NOS: 5178- |
| | | 5187 |
| FCGBP | Fc fragment of IgG binding protein | SEQ ID NOS: 5188- |
| | | 5190 |
| FCGR1B | Fc fragment of IgG, high affinity Ib, | SEQ ID NOS: 5191- |
| | receptor (CD64) | 5196 |
| FCGR3A | Fc fragment of IgG, low affinity IIIa, | SEQ ID NOS: 5197- |
| | receptor (CD16a) | 5203 |
| FCGRT | Fc fragment of IgG, receptor, transporter, | SEQ ID NOS: 5204- |
| | alpha | 5214 |
| FCMR | Fc fragment of IgM receptor | SEQ ID NOS: 5215- |
| | | 5221 |
| FCN1 | Ficolin (collagen/fibrinogen domain | SEQ ID NOS: 5222- |
| | containing) 1 | 5223 |
| FCN2 | Ficolin (collagen/fibrinogen domain | SEQ ID NOS: 5224- |
| | containing lectin) 2 | 5225 |
| FCN3 | Ficolin (collagen/fibrinogen domain | SEQ ID NOS: 5226- |
| | containing) 3 | 5227 |
| FCRL1 | Fc receptor-like 1 | SEQ ID NOS: 5228- |
| | | 5230 |
| FCRL3 | Fc receptor-like 3 | SEQ ID NOS: 5231- |
| | | 5236 |
| FCRL5 | Fc receptor-like 5 | SEQ ID NOS: 5237- |
| | | 5239 |
| FCRLA | Fc receptor-like A | SEQ ID NOS: 5240- |
| | | 5251 |
| FCRLB | Fc receptor-like B | SEQ ID NOS: 5252- |
| | | 5256 |
| FDCSP | Follicular dendritic cell secreted protein | SEQ ID NO: 5257 |
| FETUB | Fetuin B | SEQ ID NOS: 5258- |
| | | 5264 |
| FGA | Fibrinogen alpha chain | SEQ ID NOS: 5265- |
| | | 5267 |
| FGB | Fibrinogen beta chain | SEQ ID NOS: 5268- |
| | | 5270 |
| FGF10 | Fibroblast growth factor 10 | SEQ ID NOS: 5271- |
| | | 5272 |
| FGF17 | Fibroblast growth factor 17 | SEQ ID NOS: 5273- |
| | | 5274 |
| FGF18 | Fibroblast growth factor 18 | SEQ ID NO: 5275 |
| FGF19 | Fibroblast growth factor 19 | SEQ ID NO: 5276 |
| FGF21 | Fibroblast growth factor 21 | SEQ ID NOS: 5277- |
| | | 5278 |
| FGF22 | Fibroblast growth factor 22 | SEQ ID NOS: 5279- |
| | | 5280 |
| FGF23 | Fibroblast growth factor 23 | SEQ ID NO: 5281 |
| FGF3 | Fibroblast growth factor 3 | SEQ ID NO: 5282 |
| FGF4 | Fibroblast growth factor 4 | SEQ ID NO: 5283 |
| FGF5 | Fibroblast growth factor 5 | SEQ ID NOS: 5284- |
| | | 5286 |
| FGF7 | Fibroblast growth factor 7 | SEQ ID NOS: 5287- |
| | | 5291 |
| FGF8 | Fibroblast growth factor 8 (androgen- | SEQ ID NOS: 5292- |
| | induced) | 5297 |
| FGFBP1 | Fibroblast growth factor binding protein 1 | SEQ ID NO: 5298 |
| FGFBP2 | Fibroblast growth factor binding protein 2 | SEQ ID NO: 5299 |
| FGFBP3 | Fibroblast growth factor binding protein 3 | SEQ ID NO: 5300 |
| FGFR1 | Fibroblast growth factor receptor 1 | SEQ ID NOS: 5301- |
| | | 5322 |
| FGFR2 | Fibroblast growth factor receptor 2 | SEQ ID NOS: 5323- |
| | | 5344 |
| FGFR3 | Fibroblast growth factor receptor 3 | SEQ ID NOS: 5345- |
| | | 5352 |
| FGFR4 | Fibroblast growth factor receptor 4 | SEQ ID NOS: 5353- |
| | | 5362 |
| FGFRL1 | Fibroblast growth factor receptor-like 1 | SEQ ID NOS: 5363- |
| | | 5368 |
| FGG | Fibrinogen gamma chain | SEQ ID NOS: 5369- |
| | | 5374 |
| FGL1 | Fibrinogen-like 1 | SEQ ID NOS: 5375- |
| | | 5381 |
| FGL2 | Fibrinogen-like 2 | SEQ ID NOS: 5382- |
| | | 5383 |
| FHL1 | Four and a half LIM domains 1 | SEQ ID NOS: 5384- |
| | | 5411 |
| FHOD3 | Formin homology 2 domain containing 3 | SEQ ID NOS: 5412- |
| | | 5418 |
| FIBIN | Fin bud initiation factor homolog | SEQ ID NO: 5419 |
| | (zebrafish) | |
| FICD | FIC domain containing | SEQ ID NOS: 5420- |
| | | 5423 |
| FIGF | C-fos induced growth factor (vascular | SEQ ID NO: 14054 |
| | endothelial growth factor D) | |
| FJX1 | Four jointed box 1 | SEQ ID NO: 5424 |
| FKBP10 | FK506 binding protein 10, 65 kDa | SEQ ID NOS: 5425- |
| | | 5430 |
| FKBP11 | FK506 binding protein 11, 19 kDa | SEQ ID NOS: 5431- |
| | | 5437 |
| FKBP14 | FK506 binding protein 14, 22 kDa | SEQ ID NOS: 5438- |
| | | 5440 |
| FKBP2 | FK506 binding protein 2, 13 kDa | SEQ ID NOS: 5441- |
| | | 5444 |
| FKBP7 | FK506 binding protein 7 | SEQ ID NOS: 5445- |
| | | 5450 |
| FKBP9 | FK506 binding protein 9, 63 kDa | SEQ ID NOS: 5451- |
| | | 5454 |
| FLT1 | Fms-related tyrosine kinase 1 | SEQ ID NOS: 5455- |
| | | 5463 |
| FLT4 | Fms-related tyrosine kinase 4 | SEQ ID NOS: 5464- |
| | | 5468 |
| FMO1 | Flavin containing monooxygenase 1 | SEQ ID NOS: 5469- |
| | | 5473 |
| FMO2 | Flavin containing monooxygenase 2 (non- | SEQ ID NOS: 5474- |
| | functional) | 5476 |
| FMO3 | Flavin containing monooxygenase 3 | SEQ ID NOS: 5477- |
| | | 5479 |
| FMO5 | Flavin containing monooxygenase 5 | SEQ ID NOS: 5480- |
| | | 5486 |
| FMOD | Fibromodulin | SEQ ID NO: 5487 |
| FN1 | Fibronectin 1 | SEQ ID NOS: 5488- |
| | | 5500 |
| FNDC1 | Fibronectin type III domain containing 1 | SEQ ID NOS: 5501- |
| | | 5502 |
| FNDC7 | Fibronectin type III domain containing 7 | SEQ ID NOS: 5503- |
| | | 5504 |
| FOCAD | Focadhesin | SEQ ID NOS: 5505- |
| | | 5511 |
| FOLR2 | Folate receptor 2 (fetal) | SEQ ID NOS: 5512- |
| | | 5521 |
| FOLR3 | Folate receptor 3 (gamma) | SEQ ID NOS: 5522- |
| | | 5526 |
| FOXRED2 | FAD-dependent oxidoreductase domain | SEQ ID NOS: 5527- |
| | containing 2 | 5530 |
| FP325331.1 | Uncharacterized protein | SEQ ID NO: 5531 |
| | UNQ6126/PRO20091 | |
| FPGS | Folylpolyglutamate synthase | SEQ ID NOS: 5539- |
| | | 5545 |
| FRAS1 | Fraser extracellular matrix complex subunit | SEQ ID NOS: 5546- |
| | 1 | 5551 |
| FREM1 | FRAS1 related extracellular matrix 1 | SEQ ID NOS: 5552- |
| | | 5556 |
| FREM3 | FRAS1 related extracellular matrix 3 | SEQ ID NO: 5557 |
| FRMPD2 | FERM and PDZ domain containing 2 | SEQ ID NOS: 5558- |
| | | 5561 |
| FRZB | Frizzled-related protein | SEQ ID NO: 5562 |
| FSHB | Follicle stimulating hormone, beta | SEQ ID NOS: 5563- |
| | polypeptide | 5565 |
| FSHR | Follicle stimulating hormone receptor | SEQ ID NOS: 5566- |
| | | 5569 |
| FST | Follistatin | SEQ ID NOS: 5570- |
| | | 5573 |
| FSTL1 | Follistatin-like 1 | SEQ ID NOS: 5574- |
| | | 5577 |
| FSTL3 | Follistatin-like 3 (secreted glycoprotein) | SEQ ID NOS: 5578- |
| | | 5583 |
| FSTL4 | Follistatin-like 4 | SEQ ID NOS: 5584- |
| | | 5586 |
| FSTL5 | Follistatin-like 5 | SEQ ID NOS: 5587- |
| | | 5589 |
| FTCDNL1 | Formiminotransferase cyclodeaminase N- | SEQ ID NOS: 5590- |
| | terminal like | 5593 |
| FUCA1 | Fucosidase, alpha-L- 1, tissue | SEQ ID NO: 5594 |
| FUCA2 | Fucosidase, alpha-L- 2, plasma | SEQ ID NOS: 5595- |
| | | 5596 |
| FURIN | Furin (paired basic amino acid cleaving | SEQ ID NOS: 5597- |
| | enzyme) | 5603 |
| FUT10 | Fucosyltransferase 10 (alpha (1,3) | SEQ ID NOS: 5604- |
| | fucosyltransferase) | 5606 |
| FUT11 | Fucosyltransferase 11 (alpha (1,3) | SEQ ID NOS: 5607- |
| | fucosyltransferase) | 5608 |
| FXN | Frataxin | SEQ ID NOS: 5609- |
| | | 5616 |
| FXR1 | Fragile X mental retardation, autosomal | SEQ ID NOS: 5617- |
| | homolog 1 | 5629 |
| FXYD3 | FXYD domain containing ion transport | SEQ ID NOS: 5630- |
| | regulator 3 | 5642 |
| GABBR1 | Gamma-aminobutyric acid (GABA) B | SEQ ID NOS: 5643- |
| | receptor, 1 | 5654 |
| GABRA1 | Gamma-aminobutyric acid (GABA) A | SEQ ID NOS: 5655- |
| | receptor, alpha 1 | 5670 |
| GABRA2 | Gamma-aminobutyric acid (GABA) A | SEQ ID NOS: 5671- |
| | receptor, alpha 2 | 5685 |
| GABRA5 | Gamma-aminobutyric acid (GABA) A | SEQ ID NOS: 5686- |
| | receptor, alpha 5 | 5694 |
| GABRG3 | Gamma-aminobutyric acid (GABA) A | SEQ ID NOS: 5695- |
| | receptor, gamma 3 | 5700 |
| GABRP | Gamma-aminobutyric acid (GABA) A | SEQ ID NOS: 5701- |
| | receptor, pi | 5709 |
| GAL | Galanin/GMAP prepropeptide | SEQ ID NO: 5710 |
| GAL3ST1 | Galactose-3-O-sulfotransferase 1 | SEQ ID NOS: 5711- |
| | | 5732 |
| GAL3ST2 | Galactose-3-O-sulfotransferase 2 | SEQ ID NO: 5733 |
| GAL3ST3 | Galactose-3-O-sulfotransferase 3 | SEQ ID NOS: 5734- |
| | | 5735 |
| GALC | Galactosylceramidase | SEQ ID NOS: 5736- |
| | | 5745 |
| GALNS | Galactosamine (N-acetyl)-6-sulfatase | SEQ ID NOS: 5746- |
| | | 5751 |
| GALNT10 | Polypeptide N- | SEQ ID NOS: 5752- |
| | acetylgalactosaminyltransferase 10 | 5755 |
| GALNT12 | Polypeptide N- | SEQ ID NOS: 5756- |
| | acetylgalactosaminyltransferase 12 | 5757 |
| GALNT15 | Polypeptide N- | SEQ ID NOS: 5758- |
| | acetylgalactosaminyltransferase 15 | 5761 |
| GALNT2 | Polypeptide N- | SEQ ID NO: 5762 |
| | acetylgalactosaminyltransferase 2 | |
| GALNT6 | Polypeptide N- | SEQ ID NOS: 5763- |
| | acetylgalactosaminyltransferase 6 | 5774 |
| GALNT8 | Polypeptide N- | SEQ ID NOS: 5775- |
| | acetylgalactosaminyltransferase 8 | 5778 |
| GALNTL6 | Polypeptide N- | SEQ ID NOS: 5779- |
| | acetylgalactosaminyltransferase-like 6 | 5782 |
| GALP | Galanin-like peptide | SEQ ID NOS: 5783- |
| | | 5785 |
| GANAB | Glucosidase, alpha; neutral AB | SEQ ID NOS: 5786- |
| | | 5794 |
| GARS | Glycyl-tRNA synthetase | SEQ ID NOS: 5795- |
| | | 5798 |
| GAS1 | Growth arrest-specific 1 | SEQ ID NO: 5799 |
| GAS6 | Growth arrest-specific 6 | SEQ ID NO: 5800 |
| GAST | Gastrin | SEQ ID NO: 5801 |
| GBA | Glucosidase, beta, acid | SEQ ID NOS: 5811- |
| | | 5814 |
| GBGT1 | Globoside alpha-1,3-N- | SEQ ID NOS: 5815- |
| | acetylgalactosaminyltransferase 1 | 5823 |
| GC | Group-specific component (vitamin D | SEQ ID NOS: 5824- |
| | binding protein) | 5828 |
| GCG | Glucagon | SEQ ID NOS: 5829- |
| | | 5830 |
| GCGR | Glucagon receptor | SEQ ID NOS: 5831- |
| | | 5833 |
| GCNT7 | Glucosaminyl (N-acetyl) transferase family | SEQ ID NOS: 5834- |
| | member 7 | 5835 |
| GCSH | Glycine cleavage system protein H | SEQ ID NOS: 5836- |
| | (aminomethyl carrier) | 5844 |
| GDF1 | Growth differentiation factor 1 | SEQ ID NO: 5845 |
| GDF10 | Growth differentiation factor 10 | SEQ ID NO: 5846 |
| GDF11 | Growth differentiation factor 11 | SEQ ID NOS: 5847- |
| | | 5848 |
| GDF15 | Growth differentiation factor 15 | SEQ ID NOS: 5849- |
| | | 5851 |
| GDF2 | Growth differentiation factor 2 | SEQ ID NO: 5852 |
| GDF3 | Growth differentiation factor 3 | SEQ ID NO: 5853 |
| GDF5 | Growth differentiation factor 5 | SEQ ID NOS: 5854- |
| | | 5855 |
| GDF6 | Growth differentiation factor 6 | SEQ ID NOS: 5856- |
| | | 5858 |
| GDF7 | Growth differentiation factor 7 | SEQ ID NO: 5859 |
| GDF9 | Growth differentiation factor 9 | SEQ ID NOS: 5860- |
| | | 5864 |
| GDNF | Glial cell derived neurotrophic factor | SEQ ID NOS: 5865- |
| | | 5872 |
| GFOD2 | Glucose-fructose oxidoreductase domain | SEQ ID NOS: 5873- |
| | containing 2 | 5878 |
| GFPT2 | Glutamine-fructose-6-phosphate | SEQ ID NOS: 5879- |
| | transaminase 2 | 5881 |
| GFRA2 | GDNF family receptor alpha 2 | SEQ ID NOS: 5882- |
| | | 5888 |
| GFRA4 | GDNF family receptor alpha 4 | SEQ ID NOS: 5889- |
| | | 5891 |
| GGA2 | Golgi-associated, gamma adaptin ear | SEQ ID NOS: 5892- |
| | containing, ARF binding protein 2 | 5900 |
| GGH | Gamma-glutamyl hydrolase (conjugase, | SEQ ID NO: 5901 |
| | folylpolygammaglutamyl hydrolase) | |
| GGT1 | Gamma-glutamyltransferase 1 | SEQ ID NOS: 5902- |
| | | 5924 |
| GGT5 | Gamma-glutamyltransferase 5 | SEQ ID NOS: 5925- |
| | | 5929 |
| GH1 | Growth hormone 1 | SEQ ID NOS: 5930- |
| | | 5934 |
| GH2 | Growth hormone 2 | SEQ ID NOS: 5935- |
| | | 5939 |
| GHDC | GH3 domain containing | SEQ ID NOS: 5940- |
| | | 5947 |
| GHRH | Growth hormone releasing hormone | SEQ ID NOS: 5948- |
| | | 5950 |
| GHRHR | Growth hormone releasing hormone | SEQ ID NOS: 5951- |
| | receptor | 5956 |
| GHRL | Ghrelin/obestatin prepropeptide | SEQ ID NOS: 5957- |
| | | 5967 |
| GIF | Gastric intrinsic factor (vitamin B synthesis) | SEQ ID NOS: 5968- |
| | | 5969 |
| GIP | Gastric inhibitory polypeptide | SEQ ID NO: 5970 |
| GKN1 | Gastrokine 1 | SEQ ID NO: 5971 |
| GKN2 | Gastrokine 2 | SEQ ID NOS: 5972- |
| | | 5973 |
| GLA | Galactosidase, alpha | SEQ ID NOS: 5974- |
| | | 5975 |
| GLB1 | Galactosidase, beta 1 | SEQ ID NOS: 5976- |
| | | 5984 |
| GLB1L | Galactosidase, beta 1-like | SEQ ID NOS: 5985- |
| | | 5992 |
| GLB1L2 | Galactosidase, beta 1-like 2 | SEQ ID NOS: 5993- |
| | | 5994 |
| GLCE | Glucuronic acid epimerase | SEQ ID NOS: 5995- |
| | | 5996 |
| GLG1 | Golgi glycoprotein 1 | SEQ ID NOS: 5997- |
| | | 6004 |
| GLIPR1 | GLI pathogenesis-related 1 | SEQ ID NOS: 6005- |
| | | 6008 |
| GLIPR1L1 | GLI pathogenesis-related 1 like 1 | SEQ ID NOS: 6009- |
| | | 6012 |
| GLIS3 | GLIS family zinc finger 3 | SEQ ID NOS: 6013- |
| | | 6021 |
| GLMP | Glycosylated lysosomal membrane protein | SEQ ID NOS: 6022- |
| | | 6030 |
| GLRB | Glycine receptor, beta | SEQ ID NOS: 6031- |
| | | 6036 |
| GLS | Glutaminase | SEQ ID NOS: 6037- |
| | | 6044 |
| GLT6D1 | Glycosyltransferase 6 domain containing 1 | SEQ ID NOS: 6045- |
| | | 6046 |
| GLTPD2 | Glycolipid transfer protein domain | SEQ ID NO: 6047 |
| | containing 2 | |
| GLUD1 | Glutamate dehydrogenase 1 | SEQ ID NO: 6048 |
| GM2A | GM2 ganglioside activator | SEQ ID NOS: 6049- |
| | | 6051 |
| GML | Glycosylphosphatidylinositol anchored | SEQ ID NOS: 6052- |
| | molecule like | 6053 |
| GNAS | GNAS complex locus | SEQ ID NOS: 6054- |
| | | 6075 |
| GNLY | Granulysin | SEQ ID NOS: 6076- |
| | | 6079 |
| GNPTG | N-acetylglucosamine-1-phosphate | SEQ ID NOS: 6080- |
| | transferase, gamma subunit | 6084 |
| GNRH1 | Gonadotropin-releasing hormone 1 | SEQ ID NOS: 6085- |
| | (luteinizing-releasing hormone) | 6086 |
| GNRH2 | Gonadotropin-releasing hormone 2 | SEQ ID NOS: 6087- |
| | | 6090 |
| GNS | Glucosamine (N-acetyl)-6-sulfatase | SEQ ID NOS: 6091- |
| | | 6096 |
| GOLM1 | Golgi membrane protein 1 | SEQ ID NOS: 6097- |
| | | 6101 |
| GORAB | Golgin, RAB6-interacting | SEQ ID NOS: 6102- |
| | | 6104 |
| GOT2 | Glutamic-oxaloacetic transaminase 2, | SEQ ID NOS: 6105- |
| | mitochondrial | 6107 |
| GP2 | Glycoprotein 2 (zymogen granule | SEQ ID NOS: 6108- |
| | membrane) | 6116 |
| GP6 | Glycoprotein VI (platelet) | SEQ ID NOS: 6117- |
| | | 6120 |
| GPC2 | Glypican 2 | SEQ ID NOS: 6121- |
| | | 6122 |
| GPC5 | Glypican 5 | SEQ ID NOS: 6123- |
| | | 6125 |
| GPC6 | Glypican 6 | SEQ ID NOS: 6126- |
| | | 6127 |
| GPD2 | Glycerol-3-phosphate dehydrogenase 2 | SEQ ID NOS: 6128- |
| | (mitochondrial) | 6136 |
| GPER1 | G protein-coupled estrogen receptor 1 | SEQ ID NOS: 6137- |
| | | 6143 |
| GPHA2 | Glycoprotein hormone alpha 2 | SEQ ID NOS: 6144- |
| | | 6146 |
| GPHB5 | Glycoprotein hormone beta 5 | SEQ ID NOS: 6147- |
| | | 6148 |
| GPIHBP1 | Glycosylphosphatidylinositol anchored high | SEQ ID NO: 6149 |
| | density lipoprotein binding protein 1 | |
| GPLD1 | Glycosylphosphatidylinositol specific | SEQ ID NO: 6150 |
| | phospholipase D1 | |
| GPNMB | Glycoprotein (transmembrane) nmb | SEQ ID NOS: 6151- |
| | | 6153 |
| GPR162 | G protein-coupled receptor 162 | SEQ ID NOS: 6154- |
| | | 6157 |
| GPX3 | Glutathione peroxidase 3 | SEQ ID NOS: 6158- |
| | | 6165 |
| GPX4 | Glutathione peroxidase 4 | SEQ ID NOS: 6166- |
| | | 6176 |
| GPX5 | Glutathione peroxidase 5 | SEQ ID NOS: 6177- |
| | | 6178 |
| GPX6 | Glutathione peroxidase 6 | SEQ ID NOS: 6179- |
| | | 6181 |
| GPX7 | Glutathione peroxidase 7 | SEQ ID NO: 6182 |
| GREM1 | Gremlin 1, DAN family BMP antagonist | SEQ ID NOS: 6183- |
| | | 6185 |
| GREM2 | Gremlin 2, DAN family BMP antagonist | SEQ ID NO: 6186 |
| GRHL3 | Grainyhead-like transcription factor 3 | SEQ ID NOS: 6187- |
| | | 6192 |
| GRIA2 | Glutamate receptor, ionotropic, AMPA 2 | SEQ ID NOS: 6193- |
| | | 6204 |
| GRIA3 | Glutamate receptor, ionotropic, AMPA 3 | SEQ ID NOS: 6205- |
| | | 6210 |
| GRIA4 | Glutamate receptor, ionotropic, AMPA 4 | SEQ ID NOS: 6211- |
| | | 6222 |
| GRIK2 | Glutamate receptor, ionotropic, kainate 2 | SEQ ID NOS: 6223- |
| | | 6231 |
| GRIN2B | Glutamate receptor, ionotropic, N-methyl | SEQ ID NOS: 6232- |
| | D-aspartate 2B | 6235 |
| GRM2 | Glutamate receptor, metabotropic 2 | SEQ ID NOS: 6236- |
| | | 6239 |
| GRM3 | Glutamate receptor, metabotropic 3 | SEQ ID NOS: 6240- |
| | | 6244 |
| GRM5 | Glutamate receptor, metabotropic 5 | SEQ ID NOS: 6245- |
| | | 6249 |
| CRN | Granulin | SEQ ID NOS: 6250- |
| | | 6265 |
| GRP | Gastrin-releasing peptide | SEQ ID NOS: 6266- |
| | | 6270 |
| GSG1 | Germ cell associated 1 | SEQ ID NOS: 6280- |
| | | 6288 |
| GSN | Gelsolin | SEQ ID NOS: 6289- |
| | | 6297 |
| GTDC1 | Glycosyltransferase-like domain containing | SEQ ID NOS: 6298- |
| | 1 | 6311 |
| GTPBP10 | GTP-binding protein 10 (putative) | SEQ ID NOS: 6312- |
| | | 6320 |
| GUCA2A | Guanylate cyclase activator 2A (guanylin) | SEQ ID NO: 6321 |
| GUCA2B | Guanylate cyclase activator 2B | SEQ ID NO: 6322 |
| | (uroguanylin) | |
| GUSB | Glucuronidase, beta | SEQ ID NOS: 6323- |
| | | 6327 |
| GVQW1 | GVQW motif containing 1 | SEQ ID NO: 6328 |
| GXYLT1 | Glucoside xylosyltransferase 1 | SEQ ID NOS: 6329- |
| | | 6330 |
| GXYLT2 | Glucoside xylosyltransferase 2 | SEQ ID NOS: 6331- |
| | | 6333 |
| GYLTL1B | Glycosyltransferase-like 1B | SEQ ID NOS: 7702- |
| | | 7707 |
| GYPB | Glycophorin B (MNS blood group) | SEQ ID NOS: 6334- |
| | | 6342 |
| GZMA | Granzyme A (granzyme 1, cytotoxic T- | SEQ ID NO: 6343 |
| | lymphocyte-associated serine esterase 3) | |
| GZMB | Granzyme B (granzyme 2, cytotoxic T- | SEQ ID NOS: 6344- |
| | lymphocyte-associated serine esterase 1) | 6352 |
| GZMH | Granzyme H (cathepsin G-like 2, protein h- | SEQ ID NOS: 6353- |
| | CCPX) | 6355 |
| GZMK | Granzyme K (granzyme 3; tryptase II) | SEQ ID NO: 6356 |
| GZMM | Granzyme M (lymphocyte met-ase 1) | SEQ ID NOS: 6357- |
| | | 6358 |
| H6PD | Hexose-6-phosphate dehydrogenase | SEQ ID NOS: 6359- |
| | (glucose 1-dehydrogenase) | 6360 |
| HABP2 | Hyaluronan binding protein 2 | SEQ ID NOS: 6361- |
| | | 6362 |
| HADHB | Hydroxyacyl-CoA dehydrogenase/3- | SEQ ID NOS: 6363- |
| | ketoacyl-CoA thiolase/enoyl-CoA hydratase | 6369 |
| | (trifunctional protein), beta subunit | |
| HAMP | Hepcidin antimicrobial peptide | SEQ ID NOS: 6370- |
| | | 6371 |
| HAPLN1 | Hyaluronan and proteoglycan link protein 1 | SEQ ID NOS: 6372- |
| | | 6378 |
| HAPLN2 | Hyaluronan and proteoglycan link protein 2 | SEQ ID NOS: 6379- |
| | | 6380 |
| HAPLN3 | Hyaluronan and proteoglycan link protein 3 | SEQ ID NOS: 6381- |
| | | 6384 |
| HAPLN4 | Hyaluronan and proteoglycan link protein 4 | SEQ ID NO: 6385 |
| HARS2 | Histidyl-tRNA synthetase 2, mitochondrial | SEQ ID NOS: 6386- |
| | | 6401 |
| HAVCR1 | Hepatitis A virus cellular receptor 1 | SEQ ID NOS: 6402- |
| | | 6406 |
| HCCS | Holocytochrome c synthase | SEQ ID NOS: 6407- |
| | | 6409 |
| HCRT | Hypocretin (orexin) neuropeptide precursor | SEQ ID NO: 6410 |
| HEATR5A | HEAT repeat containing 5A | SEQ ID NOS: 6414- |
| | | 6420 |
| HEPH | Hephaestin | SEQ ID NOS: 6421- |
| | | 6428 |
| HEXA | Hexosaminidase A (alpha polypeptide) | SEQ ID NOS: 6429- |
| | | 6438 |
| HEXB | Hexosaminidase B (beta polypeptide) | SEQ ID NOS: 6439- |
| | | 6444 |
| HFE2 | Hemochromatosis type 2 (juvenile) | SEQ ID NOS: 6445- |
| | | 6451 |
| HGF | Hepatocyte growth factor (hepapoietin A; | SEQ ID NOS: 6452- |
| | scatter factor) | 6462 |
| HGFAC | HGF activator | SEQ ID NOS: 6463- |
| | | 6464 |
| HHIP | Hedgehog interacting protein | SEQ ID NOS: 6465- |
| | | 6466 |
| HHIPL1 | HHIP-like 1 | SEQ ID NOS: 6467- |
| | | 6468 |
| HHIPL2 | HHIP-like 2 | SEQ ID NO: 6469 |
| HHLA1 | HERV-H LTR-associating 1 | SEQ ID NOS: 6470- |
| | | 6471 |
| HHLA2 | HERV-H LTR-associating 2 | SEQ ID NOS: 6472- |
| | | 6482 |
| HIBADH | 3-hydroxyisobutyrate dehydrogenase | SEQ ID NOS: 6483- |
| | | 6485 |
| HINT2 | Histidine triad nucleotide binding protein 2 | SEQ ID NO: 6486 |
| HLA-A | Major histocompatibility complex, class I, | SEQ ID NOS: 6487- |
| | A | 6491 |
| HLA-C | Major histocompatibility complex, class I, C | SEQ ID NOS: 6492- |
| | | 6496 |
| HLA-DOA | Major histocompatibility complex, class II, | SEQ ID NOS: 6497- |
| | DO alpha | 6498 |
| HLA-DPA1 | Major histocompatibility complex, class II, | SEQ ID NOS: 6499- |
| | DP alpha 1 | 6502 |
| HLA-DQA1 | Major histocompatibility complex, class II, | SEQ ID NOS: 6503- |
| | DQ alpha 1 | 6508 |
| HLA-DQB1 | Major histocompatibility complex, class II, | SEQ ID NOS: 6509- |
| | DQ beta 1 | 6514 |
| HLA-DQB2 | Major histocompatibility complex, class II, | SEQ ID NOS: 6515- |
| | DQ beta 2 | 6518 |
| HMCN1 | Hemicentin 1 | SEQ ID NOS: 6519- |
| | | 6520 |
| HMCN2 | Hemicentin 2 | SEQ ID NOS: 6521- |
| | | 6524 |
| HMGCL | 3-hydroxymethyl-3-methylglutaryl-CoA | SEQ ID NOS: 6525- |
| | lyase | 6528 |
| HMHA1 | Histocompatibility (minor) HA-1 | SEQ ID NOS: 1034- |
| | | 1042 |
| HMSD | Histocompatibility (minor) serpin domain | SEQ ID NOS: 6529- |
| | containing | 6530 |
| HP | Haptoglobin | SEQ ID NOS: 6531- |
| | | 6544 |
| HPR | Haptoglobin-related protein | SEQ ID NOS: 6545- |
| | | 6547 |
| HPSE | Heparanase | SEQ ID NOS: 6548- |
| | | 6554 |
| HPSE2 | Heparanase 2 (inactive) | SEQ ID NOS: 6555- |
| | | 6560 |
| HPX | Hemopexin | SEQ ID NOS: 6561- |
| | | 6562 |
| HRC | Histidine rich calcium binding protein | SEQ ID NOS: 6563- |
| | | 6565 |
| HRG | Histidine-rich glycoprotein | SEQ ID NO: 6566 |
| HRSP12 | Heat-responsive protein 12 | SEQ ID NOS: 11389- |
| | | 11392 |
| HS2ST1 | Heparan sulfate 2-O-sulfotransferase 1 | SEQ ID NOS: 6567- |
| | | 6569 |
| HS3ST1 | Heparan sulfate (glucosamine) 3-O- | SEQ ID NOS: 6570- |
| | sulfotransferase 1 | 6572 |
| HS6ST1 | Heparan sulfate 6-O-sulfotransferase 1 | SEQ ID NO: 6573 |
| HS6ST3 | Heparan sulfate 6-O-sulfotransferase 3 | SEQ ID NOS: 6574- |
| | | 6575 |
| HSD11B1L | Hydroxysteroid (11-beta) dehydrogenase 1- | SEQ ID NOS: 6576- |
| | like | 6594 |
| HSD17B11 | Hydroxysteroid (17-beta) dehydrogenase 11 | SEQ ID NOS: 6595- |
| | | 6596 |
| HSD17B7 | Hydroxysteroid (17-beta) dehydrogenase 7 | SEQ ID NOS: 6597- |
| | | 6601 |
| HSP90B1 | Heat shock protein 90 kDa beta (Grp94), | SEQ ID NOS: 6602- |
| | member 1 | 6607 |
| HSPA13 | Heat shock protein 70 kDa family, member | SEQ ID NO: 6608 |
| | 13 | |
| HSPA5 | Heat shock 70 kDa protein 5 (glucose- | SEQ ID NO: 6609 |
| | regulated protein, 78 kDa) | |
| HSPG2 | Heparan sulfate proteoglycan 2 | SEQ ID NOS: 6610- |
| | | 6614 |
| HTATIP2 | HIV-1 Tat interactive protein 2, 30 kDa | SEQ ID NOS: 6615- |
| | | 6622 |
| HTN1 | Histatin 1 | SEQ ID NOS: 6623- |
| | | 6625 |
| HTN3 | Histatin 3 | SEQ ID NOS: 6626- |
| | | 6628 |
| HTRA1 | HtrA serine peptidase 1 | SEQ ID NOS: 6629- |
| | | 6630 |
| HTRA3 | HtrA serine peptidase 3 | SEQ ID NOS: 6631- |
| | | 6632 |
| HTRA4 | HtrA serine peptidase 4 | SEQ ID NO: 6633 |
| HYAL1 | Hyaluronoglucosaminidase 1 | SEQ ID NOS: 6634- |
| | | 6642 |
| HYAL2 | Hyaluronoglucosaminidase 2 | SEQ ID NOS: 6643- |
| | | 6651 |
| HYAL3 | Hyaluronoglucosaminidase 3 | SEQ ID NOS: 6652- |
| | | 6658 |
| HYOU1 | Hypoxia up-regulated 1 | SEQ ID NOS: 6659- |
| | | 6673 |
| IAPP | Islet amyloid polypeptide | SEQ ID NOS: 6674- |
| | | 6678 |
| IBSP | Integrin-binding sialoprotein | SEQ ID NO: 6679 |
| ICAM1 | Intercellular adhesion molecule 1 | SEQ ID NOS: 6680- |
| | | 6682 |
| ICAM2 | Intercellular adhesion molecule 2 | SEQ ID NOS: 6683- |
| | | 6693 |
| ICAM4 | Intercellular adhesion molecule 4 | SEQ ID NOS: 6694- |
| | (Landsteiner-Wiener blood group) | 6696 |
| ID1 | Inhibitor of DNA binding 1, dominant | SEQ ID NOS: 6697- |
| | negative helix-loop-helix protein | 6698 |
| IDE | Insulin-degrading enzyme | SEQ ID NOS: 6699- |
| | | 6702 |
| IDNK | IdnK, gluconokinase homolog (E. coli) | SEQ ID NOS: 6703- |
| | | 6708 |
| IDS | Iduronate 2-sulfatase | SEQ ID NOS: 6709- |
| | | 6714 |
| IDUA | Iduronidase, alpha-L- | SEQ ID NOS: 6715- |
| | | 6720 |
| IFI27L2 | Interferon, alpha-inducible protein 27-like 2 | SEQ ID NOS: 6721- |
| | | 6722 |
| IFI30 | Interferon, gamma-inducible protein 30 | SEQ ID NOS: 6723- |
| | | 6724 |
| IFNA1 | Interferon, alpha 1 | SEQ ID NO: 6725 |
| IFNA10 | Interferon, alpha 10 | SEQ ID NO: 6726 |
| IFNA13 | Interferon, alpha 13 | SEQ ID NOS: 6727- |
| | | 6728 |
| IFNA14 | Interferon, alpha 14 | SEQ ID NO: 6729 |
| IFNA16 | Interferon, alpha 16 | SEQ ID NO: 6730 |
| IFNA17 | Interferon, alpha 17 | SEQ ID NO: 6731 |
| IFNA2 | Interferon, alpha 2 | SEQ ID NO: 6732 |
| IFNA21 | Interferon, alpha 21 | SEQ ID NO: 6733 |
| IFNA4 | Interferon, alpha 4 | SEQ ID NO: 6734 |
| IFNA5 | Interferon, alpha 5 | SEQ ID NO: 6735 |
| IFNA6 | Interferon, alpha 6 | SEQ ID NOS: 6736- |
| | | 6737 |
| IFNA7 | Interferon, alpha 7 | SEQ ID NO: 6738 |
| IFNA8 | Interferon, alpha 8 | SEQ ID NO: 6739 |
| IFNAR1 | Interferon (alpha, beta and omega) receptor | SEQ ID NOS: 6740- |
| | 1 | 6741 |
| IFNB1 | Interferon, beta 1, fibroblast | SEQ ID NO: 6742 |
| IFNE | Interferon, epsilon | SEQ ID NO: 6743 |
| IFNG | Interferon, gamma | SEQ ID NO: 6744 |
| IFNGR1 | Interferon gamma receptor 1 | SEQ ID NOS: 6745- |
| | | 6755 |
| IFNL1 | Interferon, lambda 1 | SEQ ID NO: 6756 |
| IFNL2 | Interferon, lambda 2 | SEQ ID NO: 6757 |
| IFNL3 | Interferon, lambda 3 | SEQ ID NOS: 6758- |
| | | 6759 |
| IFNLR1 | Interferon, lambda receptor 1 | SEQ ID NOS: 6760- |
| | | 6764 |
| IFNW1 | Interferon, omega 1 | SEQ ID NO: 6765 |
| IGF1 | Insulin-like growth factor 1 (somatomedin | SEQ ID NOS: 6766- |
| | C) | 6771 |
| IGF2 | Insulin-like growth factor 2 | SEQ ID NOS: 6772- |
| | | 6779 |
| IGFALS | Insulin-like growth factor binding protein, | SEQ ID NOS: 6780- |
| | acid labile subunit | 6782 |
| IGFBP1 | Insulin-like growth factor binding protein 1 | SEQ ID NOS: 6783- |
| | | 6785 |
| IGFBP2 | Insulin-like growth factor binding protein 2, | SEQ ID NOS: 6786- |
| | 36 kDa | 6789 |
| IGFBP3 | Insulin-like growth factor binding protein 3 | SEQ ID NOS: 6790- |
| | | 6797 |
| IGFBP4 | Insulin-like growth factor binding protein 4 | SEQ ID NO: 6798 |
| IGFBP5 | Insulin-like growth factor binding protein 5 | SEQ ID NOS: 6799- |
| | | 6800 |
| IGFBP6 | Insulin-like growth factor binding protein 6 | SEQ ID NOS: 6801- |
| | | 6803 |
| IGFBP7 | Insulin-like growth factor binding protein 7 | SEQ ID NOS: 6804- |
| | | 6805 |
| IGFBPL1 | Insulin-like growth factor binding protein- | SEQ ID NO: 6806 |
| | like 1 | |
| IGFL1 | IGF-like family member 1 | SEQ ID NO: 6807 |
| IGFL2 | IGF-like family member 2 | SEQ ID NOS: 6808- |
| | | 6810 |
| IGFL3 | IGF-like family member 3 | SEQ ID NO: 6811 |
| IGFLR1 | IGF-like family receptor 1 | SEQ ID NOS: 6812- |
| | | 6820 |
| IGIP | IgA-inducing protein | SEQ ID NO: 6821 |
| IGLON5 | IgLON family member 5 | SEQ ID NO: 6822 |
| IGSF1 | Immunoglobulin superfamily, member 1 | SEQ ID NOS: 6823- |
| | | 6828 |
| IGSF10 | Immunoglobulin superfamily, member 10 | SEQ ID NOS: 6829- |
| | | 6830 |
| IGSF11 | Immunoglobulin superfamily, member 11 | SEQ ID NOS: 6831- |
| | | 6838 |
| IGSF21 | Immunoglobin superfamily, member 21 | SEQ ID NO: 6839 |
| IGSF8 | Immunoglobulin superfamily, member 8 | SEQ ID NOS: 6840- |
| | | 6843 |
| IGSF9 | Immunoglobulin superfamily, member 9 | SEQ ID NOS: 6844- |
| | | 6846 |
| IHH | Indian hedgehog | SEQ ID NO: 6847 |
| IL10 | Interleukin 10 | SEQ ID NOS: 6848- |
| | | 6849 |
| IL11 | Interleukin 11 | SEQ ID NOS: 6850- |
| | | 6853 |
| IL11RA | Interleukin 11 receptor, alpha | SEQ ID NOS: 6854- |
| | | 6864 |
| IL12B | Interleukin 12B | SEQ ID NO: 6865 |
| IL12RB1 | Interleukin 12 receptor, beta 1 | SEQ ID NOS: 6866- |
| | | 6871 |
| IL12RB2 | Interleukin 12 receptor, beta 2 | SEQ ID NOS: 6872- |
| | | 6876 |
| IL13 | Interleukin 13 | SEQ ID NOS: 6877- |
| | | 6878 |
| IL13RA1 | Interleukin 13 receptor, alpha 1 | SEQ ID NOS: 6879- |
| | | 6880 |
| IL15RA | Interleukin 15 receptor, alpha | SEQ ID NOS: 6881- |
| | | 6898 |
| IL17A | Interleukin 17A | SEQ ID NO: 6899 |
| IL17B | Interleukin 17B | SEQ ID NO: 6900 |
| IL17C | Interleukin 17C | SEQ ID NO: 6901 |
| IL17D | Interleukin 17D | SEQ ID NOS: 6902- |
| | | 6904 |
| IL17F | Interleukin 17F | SEQ ID NO: 6905 |
| IL17RA | Interleukin 17 receptor A | SEQ ID NOS: 6906- |
| | | 6907 |
| IL17RC | Interleukin 17 receptor C | SEQ ID NOS: 6908- |
| | | 6923 |
| IL17RE | Interleukin 17 receptor E | SEQ ID NOS: 6924- |
| | | 6930 |
| IL18BP | Interleukin 18 binding protein | SEQ ID NOS: 6931- |
| | | 6941 |
| IL18R1 | Interleukin 18 receptor 1 | SEQ ID NOS: 6942- |
| | | 6945 |
| IL18RAP | Interleukin 18 receptor accessory protein | SEQ ID NOS: 6946- |
| | | 6948 |
| IL19 | Interleukin 19 | SEQ ID NOS: 6949- |
| | | 6951 |
| IL1R1 | Interleukin 1 receptor, type I | SEQ ID NOS: 6952- |
| | | 6964 |
| IL1R2 | Interleukin 1 receptor, type II | SEQ ID NOS: 6965- |
| | | 6968 |
| IL1RAP | Interleukin 1 receptor accessory protein | SEQ ID NOS: 6969- |
| | | 6982 |
| IL1RL1 | Interleukin 1 receptor-like 1 | SEQ ID NOS: 6983- |
| | | 6988 |
| IL1RL2 | Interleukin 1 receptor-like 2 | SEQ ID NOS: 6989- |
| | | 6991 |
| IL1RN | Interleukin 1 receptor antagonist | SEQ ID NOS: 6992- |
| | | 6996 |
| IL2 | Interleukin 2 | SEQ ID NO: 6997 |
| IL20 | Interleukin 20 | SEQ ID NOS: 6998- |
| | | 7000 |
| IL20RA | Interleukin 20 receptor, alpha | SEQ ID NOS: 7001- |
| | | 7007 |
| IL21 | Interleukin 21 | SEQ ID NOS: 7008- |
| | | 7009 |
| IL22 | Interleukin 22 | SEQ ID NOS: 7010- |
| | | 7011 |
| IL22RA2 | Interleukin 22 receptor, alpha 2 | SEQ ID NOS: 7012- |
| | | 7014 |
| IL23A | Interleukin 23, alpha subunit p19 | SEQ ID NO: 7015 |
| IL24 | Interleukin 24 | SEQ ID NOS: 7016- |
| | | 7021 |
| IL25 | Interleukin 25 | SEQ ID NOS: 7022- |
| | | 7023 |
| IL26 | Interleukin 26 | SEQ ID NO: 7024 |
| IL27 | Interleukin 27 | SEQ ID NOS: 7025- |
| | | 7026 |
| IL2RB | Interleukin 2 receptor, beta | SEQ ID NOS: 7027- |
| | | 7031 |
| IL3 | Interleukin 3 | SEQ ID NO: 7032 |
| IL31 | Interleukin 31 | SEQ ID NO: 7033 |
| IL31RA | Interleukin 31 receptor A | SEQ ID NOS: 7034- |
| | | 7041 |
| IL32 | Interleukin 32 | SEQ ID NOS: 7042- |
| | | 7071 |
| IL34 | Interleukin 34 | SEQ ID NOS: 7072- |
| | | 7075 |
| IL3RA | Interleukin 3 receptor, alpha (low affinity) | SEQ ID NOS: 7076- |
| | | 7078 |
| IL4 | Interleukin 4 | SEQ ID NOS: 7079- |
| | | 7081 |
| IL4I1 | Interleukin 4 induced 1 | SEQ ID NOS: 7082- |
| | | 7089 |
| IL4R | Interleukin 4 receptor | SEQ ID NOS: 7090- |
| | | 7103 |
| IL5 | Interleukin 5 | SEQ ID NOS: 7104- |
| | | 7105 |
| IL5RA | Interleukin 5 receptor, alpha | SEQ ID NOS: 7106- |
| | | 7115 |
| IL6 | Interleukin 6 | SEQ ID NOS: 7116- |
| | | 7122 |
| IL6R | Interleukin 6 receptor | SEQ ID NOS: 7123- |
| | | 7128 |
| IL6ST | Interleukin 6 signal transducer | SEQ ID NOS: 7129- |
| | | 7138 |
| IL7 | Interleukin 7 | SEQ ID NOS: 7139- |
| | | 7146 |
| IL7R | Interleukin 7 receptor | SEQ ID NOS: 7147- |
| | | 7153 |
| IL9 | Interleukin 9 | SEQ ID NO: 7154 |
| ILDR1 | Immunoglobulin-like domain containing | SEQ ID NOS: 7155- |
| | receptor 1 | 7159 |
| ILDR2 | Immunoglobulin-like domain containing | SEQ ID NOS: 7160- |
| | receptor 2 | 7166 |
| IMP4 | IMP4, U3 small nucleolar ribonucleoprotein | SEQ ID NOS: 7167- |
| | | 7172 |
| IMPG1 | Interphotoreceptor matrix proteoglycan 1 | SEQ ID NOS: 7173- |
| | | 7176 |
| INHA | Inhibin, alpha | SEQ ID NO: 7177 |
| INHBA | Inhibin, beta A | SEQ ID NOS: 7178- |
| | | 7180 |
| INHBB | Inhibin, beta B | SEQ ID NO: 7181 |
| INHBC | Inhibin, beta C | SEQ ID NO: 7182 |
| INHBE | Inhibin, beta E | SEQ ID NOS: 7183- |
| | | 7184 |
| INPP5A | Inositol polyphosphate-5-phosphatase A | SEQ ID NOS: 7185- |
| | | 7189 |
| INS | Insulin | SEQ ID NOS: 7190- |
| | | 7194 |
| INS-IGF2 | INS-IGF2 readthrough | SEQ ID NOS: 7195- |
| | | 7196 |
| INSL3 | Insulin-like 3 (Leydig cell) | SEQ ID NOS: 7197- |
| | | 7199 |
| INSL4 | Insulin-like 4 (placenta) | SEQ ID NO: 7200 |
| INSL5 | Insulin-like 5 | SEQ ID NO: 7201 |
| INSL6 | Insulin-like 6 | SEQ ID NO: 7202 |
| INTS3 | Integrator complex subunit 3 | SEQ ID NOS: 7203- |
| | | 7208 |
| IPO11 | Importin 11 | SEQ ID NOS: 7209- |
| | | 7217 |
| IPO9 | Importin 9 | SEQ ID NOS: 7218- |
| | | 7219 |
| IQCF6 | IQ motif containing F6 | SEQ ID NOS: 7220- |
| | | 7221 |
| IRAK3 | Interleukin-1 receptor-associated kinase 3 | SEQ ID NOS: 7222- |
| | | 7224 |
| IRS4 | Insulin receptor substrate 4 | SEQ ID NO: 7225 |
| ISLR | Immunoglobulin superfamily containing | SEQ ID NOS: 7226- |
| | leucine-rich repeat | 7229 |
| ISLR2 | Immunoglobulin superfamily containing | SEQ ID NOS: 7230- |
| | leucine-rich repeat 2 | 7239 |
| ISM1 | Isthmin 1, angiogenesis inhibitor | SEQ ID NO: 7240 |
| ISM2 | Isthmin 2 | SEQ ID NOS: 7241- |
| | | 7246 |
| ITGA4 | Integrin, alpha 4 (antigen CD49D, alpha 4 | SEQ ID NOS: 7247- |
| | subunit of VLA-4 receptor) | 7249 |
| ITGA9 | Integrin, alpha 9 | SEQ ID NOS: 7250- |
| | | 7252 |
| ITGAL | Integrin, alpha L (antigen CD11A (p180), | SEQ ID NOS: 7253- |
| | lymphocyte function-associated antigen 1; | 7262 |
| | alpha polypeptide) | |
| ITGAX | Integrin, alpha X (complement component 3 | SEQ ID NOS: 7263- |
| | receptor 4 subunit) | 7265 |
| ITGB1 | Integrin, beta 1 (fibronectin receptor, beta | SEQ ID NOS: 7266- |
| | polypeptide, antigen CD29 includes MDF2, | 7281 |
| | MSK12) | |
| ITGB2 | Integrin, beta 2 (complement component 3 | SEQ ID NOS: 7282- |
| | receptor 3 and 4 subunit) | 7298 |
| ITGB3 | Integrin, beta 3 (platelet glycoprotein IIIa, | SEQ ID NOS: 7299- |
| | antigen CD61) | 7301 |
| ITGB7 | Integrin, beta 7 | SEQ ID NOS: 7302- |
| | | 7309 |
| ITGBL1 | Integrin, beta-like 1 (with EGF-like repeat | SEQ ID NOS: 7310- |
| | domains) | 7315 |
| ITIH1 | Inter-alpha-trypsin inhibitor heavy chain 1 | SEQ ID NOS: 7316- |
| | | 7321 |
| ITIH2 | Inter-alpha-trypsin inhibitor heavy chain 2 | SEQ ID NOS: 7322- |
| | | 7324 |
| ITIH3 | Inter-alpha-trypsin inhibitor heavy chain 3 | SEQ ID NOS: 7325- |
| | | 7327 |
| ITIH4 | Inter-alpha-trypsin inhibitor heavy chain | SEQ ID NOS: 7328- |
| | family, member 4 | 7331 |
| ITIH5 | Inter-alpha-trypsin inhibitor heavy chain | SEQ ID NOS: 7332- |
| | family, member 5 | 7335 |
| ITIH6 | Inter-alpha-trypsin inhibitor heavy chain | SEQ ID NO: 7336 |
| | family, member 6 | |
| ITLN1 | Intelectin 1 (galactofuranose binding) | SEQ ID NO: 7337 |
| ITLN2 | Intelectin 2 | SEQ ID NO: 7338 |
| IZUMO1R | IZUMO1 receptor, JUNO | SEQ ID NOS: 7339- |
| | | 7340 |
| IZUMO4 | IZUMO family member 4 | SEQ ID NOS: 7341- |
| | | 7347 |
| JCHAIN | Joining chain of multimeric IgA and IgM | SEQ ID NOS: 7357- |
| | | 7362 |
| JMJD8 | Jumonji domain containing 8 | SEQ ID NOS: 7363- |
| | | 7367 |
| JSRP1 | Junctional sarcoplasmic reticulum protein 1 | SEQ ID NO: 7368 |
| KANSL2 | KAT8 regulatory NSL complex subunit 2 | SEQ ID NOS: 7369- |
| | | 7379 |
| KAZALD1 | Kazal-type serine peptidase inhibitor | SEQ ID NO: 7380 |
| | domain 1 | |
| KCNIP3 | Kv channel interacting protein 3, calsenilin | SEQ ID NOS: 7381- |
| | | 7383 |
| KCNK7 | Potassium channel, two pore domain | SEQ ID NOS: 7384- |
| | subfamily K, member 7 | 7389 |
| KCNN4 | Potassium channel, calcium activated | SEQ ID NOS: 7390- |
| | intermediate/small conductance subfamily | 7395 |
| | N alpha, member 4 | |
| KCNU1 | Potassium channel, subfamily U, member 1 | SEQ ID NOS: 7396- |
| | | 7400 |
| KCP | Kielin/chordin-like protein | SEQ ID NOS: 7401- |
| | | 7404 |
| KDELC1 | KDEL (Lys-Asp-Glu-Leu) containing 1 | SEQ ID NO: 7405 |
| KDELC2 | KDEL (Lys-Asp-Glu-Leu) containing 2 | SEQ ID NOS: 7406- |
| | | 7409 |
| KDM1A | Lysine (K)-specific demethylase 1A | SEQ ID NOS: 7410- |
| | | 7413 |
| KDM3B | Lysine (K)-specific demethylase 3B | SEQ ID NOS: 7414- |
| | | 7417 |
| KDM6A | Lysine (K)-specific demethylase 6A | SEQ ID NOS: 7418- |
| | | 7427 |
| KDM7A | Lysine (K)-specific demethylase 7A | SEQ ID NOS: 7428- |
| | | 7429 |
| KDSR | 3-ketodihydrosphingosine reductase | SEQ ID NOS: 7430- |
| | | 7436 |
| KERA | Keratocan | SEQ ID NO: 7437 |
| KIAA0100 | KJAA0100 | SEQ ID NOS: 7438- |
| | | 7443 |
| KIAA0319 | KJAA0319 | SEQ ID NOS: 7444- |
| | | 7449 |
| KIAA1324 | KIAA1324 | SEQ ID NOS: 7450- |
| | | 7458 |
| KIFC2 | Kinesin family member C2 | SEQ ID NOS: 7459- |
| | | 7461 |
| KIR2DL4 | Killer cell immunoglobulin-like receptor, | SEQ ID NOS: 7462- |
| | two domains, long cytoplasmic tail, 4 | 7468 |
| KIR3DX1 | Killer cell immunoglobulin-like receptor, | SEQ ID NOS: 7469- |
| | three domains, X1 | 7473 |
| KIRREL2 | Kin of IRRE like 2 (Drosophila) | SEQ ID NOS: 7474- |
| | | 7478 |
| KISS1 | KiSS-1 metastasis-suppressor | SEQ ID NOS: 7479- |
| | | 7480 |
| KLHL11 | Kelch-like family member 11 | SEQ ID NO: 7481 |
| KLHL22 | Kelch-like family member 22 | SEQ ID NOS: 7482- |
| | | 7488 |
| KLK1 | Kallikrein 1 | SEQ ID NOS: 7489- |
| | | 7490 |
| KLK10 | Kallikrein-related peptidase 10 | SEQ ID NOS: 7491- |
| | | 7495 |
| KLK11 | Kallikrein-related peptidase 11 | SEQ ID NOS: 7496- |
| | | 7504 |
| KLK12 | Kallikrein-related peptidase 12 | SEQ ID NOS: 7505- |
| | | 7511 |
| KLK13 | Kallikrein-related peptidase 13 | SEQ ID NOS: 7512- |
| | | 7520 |
| KLK14 | Kallikrein-related peptidase 14 | SEQ ID NOS: 7521- |
| | | 7522 |
| KLK15 | Kallikrein-related peptidase 15 | SEQ ID NOS: 7523- |
| | | 7527 |
| KLK2 | Kallikrein-related peptidase 2 | SEQ ID NOS: 7528- |
| | | 7540 |
| KLK3 | Kallikrein-related peptidase 3 | SEQ ID NOS: 7541- |
| | | 7552 |
| KLK4 | Kallikrein-related peptidase 4 | SEQ ID NOS: 7553- |
| | | 7557 |
| KLK5 | Kallikrein-related peptidase 5 | SEQ ID NOS: 7558- |
| | | 7561 |
| KLK6 | Kallikrein-related peptidase 6 | SEQ ID NOS: 7562- |
| | | 7568 |
| KLK7 | Kallikrein-related peptidase 7 | SEQ ID NOS: 7569- |
| | | 7573 |
| KLK8 | Kallikrein-related peptidase 8 | SEQ ID NOS: 7574- |
| | | 7581 |
| KLK9 | Kallikrein-related peptidase 9 | SEQ ID NOS: 7582- |
| | | 7583 |
| KLKB1 | Kallikrein B, plasma (Fletcher factor) 1 | SEQ ID NOS: 7584- |
| | | 7588 |
| KNDC1 | Kinase non-catalytic C-lobe domain | SEQ ID NOS: 7593- |
| | (KIND) containing 1 | 7594 |
| KNG1 | Kininogen 1 | SEQ ID NOS: 7595- |
| | | 7599 |
| KRBA2 | KRAB-A domain containing 2 | SEQ ID NOS: 7600- |
| | | 7603 |
| KREMEN2 | Kringle containing transmembrane protein 2 | SEQ ID NOS: 7604- |
| | | 7609 |
| KRTDAP | Keratinocyte differentiation-associated | SEQ ID NOS: 7610- |
| | protein | 7611 |
| L1CAM | L1 cell adhesion molecule | SEQ ID NOS: 7612- |
| | | 7621 |
| L3MBTL2 | L(3)mbt-like 2 (Drosophila) | SEQ ID NOS: 7622- |
| | | 7626 |
| LA16c- | | SEQ ID NO: 72 |
| 380H5.3 | | |
| LACE1 | Lactation elevated 1 | SEQ ID NOS: 580-583 |
| LACRT | Lacritin | SEQ ID NOS: 7627- |
| | | 7629 |
| LACTB | Lactamase, beta | SEQ ID NOS: 7630- |
| | | 7632 |
| LAG3 | Lymphocyte-activation gene 3 | SEQ ID NOS: 7633- |
| | | 7634 |
| LAIR2 | Leukocyte-associated immunoglobulin-like | SEQ ID NOS: 7635- |
| | receptor 2 | 7638 |
| LALBA | Lactalbumin, alpha- | SEQ ID NOS: 7639- |
| | | 7640 |
| LAMA1 | Laminin, alpha 1 | SEQ ID NOS: 7641- |
| | | 7642 |
| LAMA2 | Laminin, alpha 2 | SEQ ID NOS: 7643- |
| | | 7646 |
| LAMA3 | Laminin, alpha 3 | SEQ ID NOS: 7647- |
| | | 7656 |
| LAMA4 | Laminin, alpha 4 | SEQ ID NOS: 7657- |
| | | 7671 |
| LAMA5 | Laminin, alpha 5 | SEQ ID NOS: 7672- |
| | | 7674 |
| LAMB1 | Laminin, beta 1 | SEQ ID NOS: 7675- |
| | | 7679 |
| LAMB2 | Laminin, beta 2 (laminin S) | SEQ ID NOS: 7680- |
| | | 7682 |
| LAMB3 | Laminin, beta 3 | SEQ ID NOS: 7683- |
| | | 7687 |
| LAMB4 | Laminin, beta 4 | SEQ ID NOS: 7688- |
| | | 7691 |
| LAMC1 | Laminin, gamma 1 (formerly LAMB2) | SEQ ID NOS: 7692- |
| | | 7693 |
| LAMC2 | Laminin, gamma 2 | SEQ ID NOS: 7694- |
| | | 7695 |
| LAMC3 | Laminin, gamma 3 | SEQ ID NOS: 7696- |
| | | 7697 |
| LAMP3 | Lysosomal-associated membrane protein 3 | SEQ ID NOS: 7698- |
| | | 7701 |
| LAT | Linker for activation of T cells | SEQ ID NOS: 7708- |
| | | 7717 |
| LAT2 | Linker for activation of T cells family, | SEQ ID NOS: 7718- |
| | member 2 | 7726 |
| LBP | Lipopolysaccharide binding protein | SEQ ID NO: 7727 |
| LCAT | Lecithin-cholesterol acyltransferase | SEQ ID NOS: 7728- |
| | | 7734 |
| LCN1 | Lipocalin 1 | SEQ ID NOS: 7735- |
| | | 7736 |
| LCN10 | Lipocalin 10 | SEQ ID NOS: 7737- |
| | | 7742 |
| LCN12 | Lipocalin 12 | SEQ ID NOS: 7743- |
| | | 7745 |
| LCN15 | Lipocalin 15 | SEQ ID NO: 7746 |
| LCN2 | Lipocalin 2 | SEQ ID NOS: 7747- |
| | | 7749 |
| LCN6 | Lipocalin 6 | SEQ ID NOS: 7750- |
| | | 7751 |
| LCN8 | Lipocalin 8 | SEQ ID NOS: 7752- |
| | | 7753 |
| LCN9 | Lipocalin 9 | SEQ ID NOS: 7754- |
| | | 7755 |
| LCORL | Ligand dependent nuclear receptor | SEQ ID NOS: 7756- |
| | corepressor-like | 7761 |
| LDLR | Low density lipoprotein receptor | SEQ ID NOS: 7762- |
| | | 7770 |
| LDLRAD2 | Low density lipoprotein receptor class A | SEQ ID NOS: 7771- |
| | domain containing 2 | 7772 |
| LEAP2 | Liver expressed antimicrobial peptide 2 | SEQ ID NO: 7773 |
| LECT2 | Leukocyte cell-derived chemotaxin 2 | SEQ ID NOS: 7774- |
| | | 7777 |
| LEFTY1 | Left-right determination factor 1 | SEQ ID NOS: 7778- |
| | | 7779 |
| LEFTY2 | Left-right determination factor 2 | SEQ ID NOS: 7780- |
| | | 7781 |
| LEP | Leptin | SEQ ID NO: 7782 |
| LFNG | LFNG O-fucosylpeptide 3-beta-N- | SEQ ID NOS: 7783- |
| | acetylglucosaminyltransferase | 7788 |
| LGALS3BP | Lectin, galactoside-binding, soluble, 3 | SEQ ID NOS: 7789- |
| | binding protein | 7803 |
| LGI1 | Leucine-rich, glioma inactivated 1 | SEQ ID NOS: 7804- |
| | | 7822 |
| LGI2 | Leucine-rich repeat LGI family, member 2 | SEQ ID NOS: 7823- |
| | | 7824 |
| LGI3 | Leucine-rich repeat LGI family, member 3 | SEQ ID NOS: 7825- |
| | | 7828 |
| LGI4 | Leucine-rich repeat LGI family, member 4 | SEQ ID NOS: 7829- |
| | | 7832 |
| LGMN | Legumain | SEQ ID NOS: 7833- |
| | | 7846 |
| LGR4 | Leucine-rich repeat containing G protein- | SEQ ID NOS: 7847- |
| | coupled receptor 4 | 7849 |
| LHB | Luteinizing hormone beta polypeptide | SEQ ID NO: 7850 |
| LHCGR | Luteinizing hormone/choriogonadotropin | SEQ ID NOS: 7851- |
| | receptor | 7855 |
| LIF | Leukemia inhibitory factor | SEQ ID NOS: 7856- |
| | | 7857 |
| LIFR | Leukemia inhibitory factor receptor alpha | SEQ ID NOS: 7858- |
| | | 7862 |
| LILRA1 | Leukocyte immunoglobulin-like receptor, | SEQ ID NOS: 7863- |
| | subfamily A (with TM domain), member 1 | 7864 |
| LILRA2 | Leukocyte immunoglobulin-like receptor, | SEQ ID NOS: 7865- |
| | subfamily A (with TM domain), member 2 | 7871 |
| LILRB3 | Leukocyte immunoglobulin-like receptor, | SEQ ID NOS: 7872- |
| | subfamily B (with TM and ITIM domains), | 7876 |
| | member 3 | |
| LIME1 | Lek interacting transmembrane adaptor 1 | SEQ ID NOS: 7877- |
| | | 7882 |
| LINGO1 | Leucine rich repeat and Ig domain | SEQ ID NOS: 7883- |
| | containing 1 | 7893 |
| LIPA | Lipase A, lysosomal acid, cholesterol | SEQ ID NOS: 7894- |
| | esterase | 7898 |
| LIPC | Lipase, hepatic | SEQ ID NOS: 7899- |
| | | 7902 |
| LIPF | Lipase, gastric | SEQ ID NOS: 7903- |
| | | 7906 |
| LIPG | Lipase, endothelial | SEQ ID NOS: 7907- |
| | | 7912 |
| LIPH | Lipase, member H | SEQ ID NOS: 7913- |
| | | 7917 |
| LIPK | Lipase, family member K | SEQ ID NO: 7918 |
| LIPM | Lipase, family member M | SEQ ID NOS: 7919- |
| | | 7920 |
| LIPN | Lipase, family member N | SEQ ID NO: 7921 |
| LMAN2 | Lectin, mannose-binding 2 | SEQ ID NOS: 7922- |
| | | 7926 |
| LMNTD1 | Lamin tail domain containing 1 | SEQ ID NOS: 7927- |
| | | 7937 |
| LNX1 | Ligand of numb-protein X 1, E3 ubiquitin | SEQ ID NOS: 7938- |
| | protein ligase | 7944 |
| LOX | Lysyl oxidase | SEQ ID NOS: 7945- |
| | | 7947 |
| LOXL1 | Lysyl oxidase-like 1 | SEQ ID NOS: 7948- |
| | | 7949 |
| LOXL2 | Lysyl oxidase-like 2 | SEQ ID NOS: 7950- |
| | | 7958 |
| LOXL3 | Lysyl oxidase-like 3 | SEQ ID NOS: 7959- |
| | | 7965 |
| LOXL4 | Lysyl oxidase-like 4 | SEQ ID NO: 7966 |
| LPA | Lipoprotein, Lp(a) | SEQ ID NOS: 7967- |
| | | 7969 |
| LPL | Lipoprotein lipase | SEQ ID NOS: 7970- |
| | | 7974 |
| LPO | Lactoperoxidase | SEQ ID NOS: 7975- |
| | | 7981 |
| LRAT | Lecithin retinol acyltransferase | SEQ ID NOS: 7982- |
| | (phosphatidylcholine--retinol O- | 7984 |
| | acyltransferase) | |
| LRCH3 | Leucine-rich repeats and calponin | SEQ ID NOS: 7985- |
| | homology (CH) domain containing 3 | 7993 |
| LRCOL1 | Leucine rich colipase-like 1 | SEQ ID NOS: 7994- |
| | | 7997 |
| LRFN4 | Leucine rich repeat and fibronectin type III | SEQ ID NOS: 7998- |
| | domain containing 4 | 7999 |
| LRFN5 | Leucine rich repeat and fibronectin type III | SEQ ID NOS: 8000- |
| | domain containing 5 | 8002 |
| LRG1 | Leucine-rich alpha-2-glycoprotein 1 | SEQ ID NO: 8003 |
| LRP1 | Low density lipoprotein receptor-related | SEQ ID NOS: 8004- |
| | protein 1 | 8009 |
| LRP11 | Low density lipoprotein receptor-related | SEQ ID NOS: 8010- |
| | protein 11 | 8011 |
| LRP1B | Low density lipoprotein receptor-related | SEQ ID NOS: 8012- |
| | protein 1B | 8015 |
| LRP2 | Low density lipoprotein receptor-related | SEQ ID NOS: 8016- |
| | protein 2 | 8017 |
| LRP4 | Low density lipoprotein receptor-related | SEQ ID NOS: 8018- |
| | protein 4 | 8019 |
| LRPAP1 | Low density lipoprotein receptor-related | SEQ ID NOS: 8020- |
| | protein associated protein 1 | 8021 |
| LRRC17 | Leucine rich repeat containing 17 | SEQ ID NOS: 8022- |
| | | 8024 |
| LRRC32 | Leucine rich repeat containing 32 | SEQ ID NOS: 8025- |
| | | 8028 |
| LRRC3B | Leucine rich repeat containing 3B | SEQ ID NOS: 8029- |
| | | 8033 |
| LRRC4B | Leucine rich repeat containing 4B | SEQ ID NOS: 8034- |
| | | 8036 |
| LRRC70 | Leucine rich repeat containing 70 | SEQ ID NOS: 8037- |
| | | 8038 |
| LRRN3 | Leucine rich repeat neuronal 3 | SEQ ID NOS: 8039- |
| | | 8042 |
| LRRTM1 | Leucine rich repeat transmembrane | SEQ ID NOS: 8043- |
| | neuronal 1 | 8049 |
| LRRTM2 | Leucine rich repeat transmembrane | SEQ ID NOS: 8050- |
| | neuronal 2 | 8052 |
| LRRTM4 | Leucine rich repeat transmembrane | SEQ ID NOS: 8053- |
| | neuronal 4 | 8058 |
| LRTM2 | Leucine-rich repeats and transmembrane | SEQ ID NOS: 8059- |
| | domains 2 | 8063 |
| LSR | Lipolysis stimulated lipoprotein receptor | SEQ ID NOS: 8064- |
| | | 8074 |
| LST1 | Leukocyte specific transcript 1 | SEQ ID NOS: 8075- |
| | | 8092 |
| LTA | Lymphotoxin alpha | SEQ ID NOS: 8093- |
| | | 8094 |
| LTBP1 | Latent transforming growth factor beta | SEQ ID NOS: 8095- |
| | binding protein 1 | 8104 |
| LTBP2 | Latent transforming growth factor beta | SEQ ID NOS: 8105- |
| | binding protein 2 | 8108 |
| LTBP3 | Latent transforming growth factor beta | SEQ ID NOS: 8109- |
| | binding protein 3 | 8121 |
| LTBP4 | Latent transforming growth factor beta | SEQ ID NOS: 8122- |
| | binding protein 4 | 8137 |
| LTBR | Lymphotoxin beta receptor (TNFR | SEQ ID NOS: 8138- |
| | superfamily, member 3) | 8143 |
| LTF | Lactotransferrin | SEQ ID NOS: 8144- |
| | | 8148 |
| LTK | Leukocyte receptor tyrosine kinase | SEQ ID NOS: 8149- |
| | | 8152 |
| LUM | Lumican | SEQ ID NO: 8153 |
| LUZP2 | Leucine zipper protein 2 | SEQ ID NOS: 8154- |
| | | 8157 |
| LVRN | Laeverin | SEQ ID NOS: 8158- |
| | | 8163 |
| LY6E | Lymphocyte antigen 6 complex, locus E | SEQ ID NOS: 8164- |
| | | 8177 |
| LY6G5B | Lymphocyte antigen 6 complex, locus G5B | SEQ ID NOS: 8178- |
| | | 8179 |
| LY6G6D | Lymphocyte antigen 6 complex, locus G6D | SEQ ID NOS: 8180- |
| | | 8181 |
| LY6G6E | Lymphocyte antigen 6 complex, locus G6E | SEQ ID NOS: 8182- |
| | (pseudogene) | 8185 |
| LY6H | Lymphocyte antigen 6 complex, locus H | SEQ ID NOS: 8186- |
| | | 8189 |
| LY6K | Lymphocyte antigen 6 complex, locus K | SEQ ID NOS: 8190- |
| | | 8193 |
| LY86 | Lymphocyte antigen 86 | SEQ ID NOS: 8195- |
| | | 8196 |
| LY96 | Lymphocyte antigen 96 | SEQ ID NOS: 8197- |
| | | 8198 |
| LYG1 | Lysozyme G-like 1 | SEQ ID NOS: 8199- |
| | | 8200 |
| LYG2 | Lysozyme G-like 2 | SEQ ID NOS: 8201- |
| | | 8206 |
| LYNX1 | Ly6/neurotoxin 1 | SEQ ID NOS: 8207- |
| | | 8211 |
| LYPD1 | LY6/PLAUR domain containing 1 | SEQ ID NOS: 8212- |
| | | 8214 |
| LYPD2 | LY6/PLAUR domain containing 2 | SEQ ID NO: 8215 |
| LYPD4 | LY6/PLAUR domain containing 4 | SEQ ID NOS: 8216- |
| | | 8218 |
| LYPD6 | LY6/PLAUR domain containing 6 | SEQ ID NOS: 8219- |
| | | 8223 |
| LYPD6B | LY6/PLAUR domain containing 6B | SEQ ID NOS: 8224- |
| | | 8230 |
| LYPD8 | LY6/PLAUR domain containing 8 | SEQ ID NOS: 8231- |
| | | 8232 |
| LYZ | Lysozyme | SEQ ID NOS: 8233- |
| | | 8235 |
| LYZL4 | Lysozyme-like 4 | SEQ ID NOS: 8236- |
| | | 8237 |
| LYZL6 | Lysozyme-like 6 | SEQ ID NOS: 8238- |
| | | 8240 |
| M6PR | Mannose-6-phosphate receptor (cation | SEQ ID NOS: 8241- |
| | dependent) | 8251 |
| MAD1L1 | MAD1 mitotic arrest deficient-like 1 (yeast) | SEQ ID NOS: 8252- |
| | | 8264 |
| MAG | Myelin associated glycoprotein | SEQ ID NOS: 8265- |
| | | 8270 |
| MAGT1 | Magnesium transporter 1 | SEQ ID NOS: 8271- |
| | | 8274 |
| MALSU1 | Mitochondrial assembly of ribosomal large | SEQ ID NO: 8275 |
| | subunit 1 | |
| MAMDC2 | MAM domain containing 2 | SEQ ID NO: 8276 |
| MAN2B1 | Mannosidase, alpha, class 2B, member 1 | SEQ ID NOS: 8277- |
| | | 8282 |
| MAN2B2 | Mannosidase, alpha, class 2B, member 2 | SEQ ID NOS: 8283- |
| | | 8285 |
| MANBA | Mannosidase, beta A, lysosomal | SEQ ID NOS: 8286- |
| | | 8299 |
| MANEAL | Mannosidase, endo-alpha-like | SEQ ID NOS: 8300- |
| | | 8304 |
| MANF | Mesencephalic astrocyte-derived | SEQ ID NOS: 8305- |
| | neurotrophic factor | 8306 |
| MANSC1 | MANSC domain containing 1 | SEQ ID NOS: 8307- |
| | | 8310 |
| MAP3K9 | Mitogen-activated protein kinase 9 | SEQ ID NOS: 8311- |
| | | 8316 |
| MASP1 | Mannan-binding lectin serine peptidase 1 | SEQ ID NOS: 8317- |
| | (C4/C2 activating component of Ra-reactive | 8324 |
| | factor) | |
| MASP2 | Mannan-binding lectin serine peptidase 2 | SEQ ID NOS: 8325- |
| | | 8326 |
| MATN1 | Matrilin 1, cartilage matrix protein | SEQ ID NO: 8327 |
| MATN2 | Matrilin 2 | SEQ ID NOS: 8328- |
| | | 8340 |
| MATN3 | Matrilin 3 | SEQ ID NOS: 8341- |
| | | 8342 |
| MATN4 | Matrilin 4 | SEQ ID NOS: 8343- |
| | | 8347 |
| MATR3 | Matrin 3 | SEQ ID NOS: 8348- |
| | | 8375 |
| MAU2 | MAU2 sister chromatid cohesion factor | SEQ ID NOS: 8376- |
| | | 8378 |
| MAZ | MYC-associated zinc finger protein (purine- | SEQ ID NOS: 8379- |
| | binding transcription factor) | 8393 |
| MBD6 | Methyl-CpG binding domain protein 6 | SEQ ID NOS: 8394- |
| | | 8405 |
| MBL2 | Mannose-binding lectin (protein C) 2, | SEQ ID NO: 8406 |
| | soluble | |
| MBNL1 | Muscleblind-like splicing regulator 1 | SEQ ID NOS: 8407- |
| | | 8425 |
| MCCC1 | Methylcrotonoyl-CoA carboxylase 1 (alpha) | SEQ ID NOS: 8426- |
| | | 8437 |
| MCCD1 | Mitochondrial coiled-coil domain 1 | SEQ ID NO: 8438 |
| MCEE | Methylmalonyl CoA epimerase | SEQ ID NOS: 8439- |
| | | 8442 |
| MCF2L | MCF.2 cell line derived transforming | SEQ ID NOS: 8443- |
| | sequence-like | 8464 |
| MCFD2 | Multiple coagulation factor deficiency 2 | SEQ ID NOS: 8465- |
| | | 8476 |
| MDFIC | MyoD family inhibitor domain containing | SEQ ID NOS: 8477- |
| | | 8484 |
| MDGA1 | MAM domain containing | SEQ ID NOS: 8485- |
| | glycosylphosphatidylinositol anchor 1 | 8490 |
| MDK | Midkine (neurite growth-promoting factor | SEQ ID NOS: 8491- |
| | 2) | 8500 |
| MED20 | Mediator complex subunit 20 | SEQ ID NOS: 8501- |
| | | 8505 |
| MEGF10 | Multiple EGF-like-domains 10 | SEQ ID NOS: 8506- |
| | | 8509 |
| MEGF6 | Multiple EGF-like-domains 6 | SEQ ID NOS: 8510- |
| | | 8513 |
| MEI1 | Meiotic double-stranded break formation | SEQ ID NOS: 8514- |
| | protein 1 | 8517 |
| MEI4 | Meiotic double-stranded break formation | SEQ ID NO: 8518 |
| | protein 4 | |
| MEIS1 | Meis homeobox 1 | SEQ ID NOS: 8519- |
| | | 8524 |
| MEIS3 | Meis homeobox 3 | SEQ ID NOS: 8525- |
| | | 8534 |
| MEPE | Matrix extracellular phosphoglycoprotein | SEQ ID NOS: 8538- |
| | | 8544 |
| MESDC2 | Mesoderm development candidate 2 | SEQ ID NOS: 8545- |
| | | 8549 |
| MEST | Mesoderm specific transcript | SEQ ID NOS: 8550- |
| | | 8563 |
| MET | MET proto-oncogene, receptor tyrosine | SEQ ID NOS: 8564- |
| | kinase | 8569 |
| METRN | Meteorin, glial cell differentiation regulator | SEQ ID NOS: 8570- |
| | | 8574 |
| METRNL | Meteorin, glial cell differentiation regulator- | SEQ ID NOS: 8575- |
| | like | 8578 |
| METTL17 | Methyltransferase like 17 | SEQ ID NOS: 8579- |
| | | 8589 |
| METTL24 | Methyltransferase like 24 | SEQ ID NO: 8590 |
| METTL7B | Methyltransferase like 7B | SEQ ID NOS: 8591- |
| | | 8592 |
| METTL9 | Methyltransferase like 9 | SEQ ID NOS: 8593- |
| | | 8601 |
| MEX3C | Mex-3 RNA binding family member C | SEQ ID NOS: 8602- |
| | | 8604 |
| MFAP2 | Microfibrillar-associated protein 2 | SEQ ID NOS: 8605- |
| | | 8606 |
| MFAP3 | Microfibrillar-associated protein 3 | SEQ ID NOS: 8607- |
| | | 8611 |
| MFAP3L | Microfibrillar-associated protein 3-like | SEQ ID NOS: 8612- |
| | | 8621 |
| MFAP4 | Microfibrillar-associated protein 4 | SEQ ID NOS: 8622- |
| | | 8624 |
| MFAP5 | Microfibrillar associated protein 5 | SEQ ID NOS: 8625- |
| | | 8635 |
| MFGE8 | Milk fat globule-EGF factor 8 protein | SEQ ID NOS: 8636- |
| | | 8642 |
| MFI2 | Antigen p97 (melanoma associated) | SEQ ID NOS: 8535- |
| | identified by monoclonal antibodies 133.2 | 8537 |
| | and 96.5 | |
| MFNG | MFNG O-fucosylpeptide 3-beta-N- | SEQ ID NOS: 8643- |
| | acetylglucosaminyltransferase | 8650 |
| MGA | MGA, MAX dimerization protein | SEQ ID NOS: 8651- |
| | | 8659 |
| MGAT2 | Mannosyl (alpha-1,6-)-glycoprotein beta- | SEQ ID NO: 8660 |
| | 1,2-N-acetylglucosaminyltransferase | |
| MGAT3 | Mannosyl (beta-1,4-)-glycoprotein beta-1,4- | SEQ ID NOS: 8661- |
| | N-acetylglucosaminyltransferase | 8663 |
| MGAT4A | Mannosyl (alpha-1,3-)-glycoprotein beta- | SEQ ID NOS: 8664- |
| | 1,4-N-acetylglucosaminyltransferase, | 8668 |
| | isozyme A | |
| MGAT4B | Mannosyl (alpha-1,3-)-glycoprotein beta- | SEQ ID NOS: 8669- |
| | 1,4-N-acetylglucosaminyltransferase, | 8679 |
| | isozyme B | |
| MGAT4D | MGAT4 family, member D | SEQ ID NOS: 8680- |
| | | 8685 |
| MGLL | Monoglyceride lipase | SEQ ID NOS: 8686- |
| | | 8695 |
| MGP | Matrix Gla protein | SEQ ID NOS: 8696- |
| | | 8698 |
| MGST2 | Microsomal glutathione S-transferase 2 | SEQ ID NOS: 8699- |
| | | 8702 |
| MIA | Melanoma inhibitory activity | SEQ ID NOS: 8703- |
| | | 8708 |
| MIA2 | Melanoma inhibitory activity 2 | SEQ ID NO: 8709 |
| MIA3 | Melanoma inhibitory activity family, | SEQ ID NOS: 8710- |
| | member 3 | 8714 |
| MICU1 | Mitochondrial calcium uptake 1 | SEQ ID NOS: 8715- |
| | | 8724 |
| MIER1 | Mesoderm induction early response 1, | SEQ ID NOS: 8725- |
| | transcriptional regulator | 8733 |
| MINOS1- | MINOS1-NBL1 readthrough | SEQ ID NOS: 8734- |
| NBL1 | | 8736 |
| MINPP1 | Multiple inositol-polyphosphate | SEQ ID NOS: 8737- |
| | phosphatase 1 | 8739 |
| MLEC | Malectin | SEQ ID NOS: 8740- |
| | | 8743 |
| MLN | Motilin | SEQ ID NOS: 8744- |
| | | 8746 |
| MLXIP | MLX interacting protein | SEQ ID NOS: 8747- |
| | | 8752 |
| MLXIPL | MLX interacting protein-like | SEQ ID NOS: 8753- |
| | | 8760 |
| MMP1 | Matrix metallopeptidase 1 | SEQ ID NO: 8761 |
| MMP10 | Matrix metallopeptidase 10 | SEQ ID NOS: 8762- |
| | | 8763 |
| MMP11 | Matrix metallopeptidase 11 | SEQ ID NOS: 8764- |
| | | 8767 |
| MMP12 | Matrix metallopeptidase 12 | SEQ ID NO: 8768 |
| MMP13 | Matrix metallopeptidase 13 | SEQ ID NOS: 8769- |
| | | 8771 |
| MMP14 | Matrix metallopeptidase 14 (membrane- | SEQ ID NOS: 8772- |
| | inserted) | 8774 |
| MMP17 | Matrix metallopeptidase 17 (membrane- | SEQ ID NOS: 8775- |
| | inserted) | 8782 |
| MMP19 | Matrix metallopeptidase 19 | SEQ ID NOS: 8783- |
| | | 8788 |
| MMP2 | Matrix metallopeptidase 2 | SEQ ID NOS: 8789- |
| | | 8796 |
| MMP20 | Matrix metallopeptidase 20 | SEQ ID NO: 8797 |
| MMP21 | Matrix metallopeptidase 21 | SEQ ID NO: 8798 |
| MMP25 | Matrix metallopeptidase 25 | SEQ ID NOS: 8799- |
| | | 8800 |
| MMP26 | Matrix metallopeptidase 26 | SEQ ID NOS: 8801- |
| | | 8802 |
| MMP27 | Matrix metallopeptidase 27 | SEQ ID NO: 8803 |
| MMP28 | Matrix metallopeptidase 28 | SEQ ID NOS: 8804- |
| | | 8809 |
| MMP3 | Matrix metallopeptidase 3 | SEQ ID NOS: 8810- |
| | | 8812 |
| MMP7 | Matrix metallopeptidase 7 | SEQ ID NO: 8813 |
| MMP8 | Matrix metallopeptidase 8 | SEQ ID NOS: 8814- |
| | | 8819 |
| MMP9 | Matrix metallopeptidase 9 | SEQ ID NO: 8820 |
| MMRN1 | Multimerin 1 | SEQ ID NOS: 8821- |
| | | 8823 |
| MMRN2 | Multimerin 2 | SEQ ID NOS: 8824- |
| | | 8828 |
| MOXD1 | Monooxygenase, DBH-like 1 | SEQ ID NOS: 8829- |
| | | 8831 |
| MPO | Myeloperoxidase | SEQ ID NOS: 8840- |
| | | 8841 |
| MPPED1 | Metallophosphoesterase domain containing | SEQ ID NOS: 8842- |
| | 1 | 8845 |
| MPZL1 | Myelin protein zero-like 1 | SEQ ID NOS: 8846- |
| | | 8850 |
| MR1 | Major histocompatibility complex, class I- | SEQ ID NOS: 8851- |
| | related | 8856 |
| MRPL2 | Mitochondrial ribosomal protein L2 | SEQ ID NOS: 8857- |
| | | 8861 |
| MRPL21 | Mitochondrial ribosomal protein L21 | SEQ ID NOS: 8862- |
| | | 8868 |
| MRPL22 | Mitochondrial ribosomal protein L22 | SEQ ID NOS: 8869- |
| | | 8873 |
| MRPL24 | Mitochondrial ribosomal protein L24 | SEQ ID NOS: 8874- |
| | | 8878 |
| MRPL27 | Mitochondrial ribosomal protein L27 | SEQ ID NOS: 8879- |
| | | 8884 |
| MRPL32 | Mitochondrial ribosomal protein L32 | SEQ ID NOS: 8885- |
| | | 8887 |
| MRPL34 | Mitochondrial ribosomal protein L34 | SEQ ID NOS: 8888- |
| | | 8892 |
| MRPL35 | Mitochondrial ribosomal protein L35 | SEQ ID NOS: 8893- |
| | | 8896 |
| MRPL52 | Mitochondrial ribosomal protein L52 | SEQ ID NOS: 8897- |
| | | 8907 |
| MRPL55 | Mitochondrial ribosomal protein L55 | SEQ ID NOS: 8908- |
| | | 8933 |
| MRPS14 | Mitochondrial ribosomal protein S14 | SEQ ID NOS: 8934- |
| | | 8935 |
| MRPS22 | Mitochondrial ribosomal protein S22 | SEQ ID NOS: 8936- |
| | | 8944 |
| MRPS28 | Mitochondrial ribosomal protein S28 | SEQ ID NOS: 8945- |
| | | 8952 |
| MS4A14 | Membrane-spanning 4-domains, subfamily | SEQ ID NOS: 8953- |
| | A, member 14 | 8963 |
| MS4A3 | Membrane-spanning 4-domains, subfamily | SEQ ID NOS: 8964- |
| | A, member 3 (hematopoietic cell-specific) | 8968 |
| MSH3 | MutS homolog 3 | SEQ ID NO: 8969 |
| MSH5 | MutS homolog 5 | SEQ ID NOS: 8970- |
| | | 8981 |
| MSLN | Mesothelin | SEQ ID NOS: 8982- |
| | | 8989 |
| MSMB | Microseminoprotein, beta- | SEQ ID NOS: 8990- |
| | | 8991 |
| MSRA | Methionine sulfoxide reductase A | SEQ ID NOS: 8992- |
| | | 8999 |
| MSRB2 | Methionine sulfoxide reductase B2 | SEQ ID NOS: 9000- |
| | | 9001 |
| MSRB3 | Methionine sulfoxide reductase B3 | SEQ ID NOS: 9002- |
| | | 9015 |
| MST1 | Macrophage stimulating 1 | SEQ ID NOS: 9016- |
| | | 9017 |
| MSTN | Myostatin | SEQ ID NO: 9018 |
| MT1G | Metallothionein 1G | SEQ ID NOS: 9019- |
| | | 9022 |
| MTHFD2 | Methylenetetrahydrofolate dehydrogenase | SEQ ID NOS: 9023- |
| | (NADP+ dependent) 2, | 9027 |
| | methenyltetrahydrofolate cyclohydrolase | |
| MTMR14 | Myotubularin related protein 14 | SEQ ID NOS: 9028- |
| | | 9038 |
| MTRNR2L11 | MT-RNR2-like 11 (pseudogene) | SEQ ID NO: 9039 |
| MTRR | 5-methyltetrahydrofolate-homocysteine | SEQ ID NOS: 9040- |
| | methyltransferase reductase | 9052 |
| MTTP | Microsomal triglyceride transfer protein | SEQ ID NOS: 9053- |
| | | 9063 |
| MTX2 | Metaxin 2 | SEQ ID NOS: 9064- |
| | | 9068 |
| MUC1 | Mucin 1, cell surface associated | SEQ ID NOS: 9069- |
| | | 9094 |
| MUC13 | Mucin 13, cell surface associated | SEQ ID NOS: 9095- |
| | | 9096 |
| MUC20 | Mucin 20, cell surface associated | SEQ ID NOS: 9097- |
| | | 9101 |
| MUC3A | Mucin 3A, cell surface associated | SEQ ID NOS: 9102- |
| | | 9104 |
| MUC5AC | Mucin 5AC, oligomeric mucus/gel-forming | SEQ ID NO: 9105 |
| MUC5B | Mucin 5B, oligomeric mucus/gel-forming | SEQ ID NOS: 9106- |
| | | 9107 |
| MUC6 | Mucin 6, oligomeric mucus/gel-forming | SEQ ID NOS: 9108- |
| | | 9111 |
| MUC7 | Mucin 7, secreted | SEQ ID NOS: 9112- |
| | | 9115 |
| MUCL1 | Mucin-like 1 | SEQ ID NOS: 9116- |
| | | 9118 |
| MXRA5 | Matrix-remodelling associated 5 | SEQ ID NO: 9119 |
| MXRA7 | Matrix-remodelling associated 7 | SEQ ID NOS: 9120- |
| | | 9126 |
| MYDGF | Myeloid-derived growth factor | SEQ ID NOS: 9127- |
| | | 9129 |
| MYL1 | Myosin, light chain 1, alkali; skeletal, fast | SEQ ID NOS: 9130- |
| | | 9131 |
| MYOC | Myocilin, trabecular meshwork inducible | SEQ ID NOS: 9132- |
| | glucocorticoid response | 9133 |
| MYRFL | Myelin regulatory factor-like | SEQ ID NOS: 9134- |
| | | 9138 |
| MZB1 | Marginal zone B and B1 cell-specific | SEQ ID NOS: 9139- |
| | protein | 9143 |
| N4BP2L2 | NEDD4 binding protein 2-like 2 | SEQ ID NOS: 9144- |
| | | 9149 |
| NAA38 | N(alpha)-acetyltransferase 38, NatC | SEQ ID NOS: 9150- |
| | auxiliary subunit | 9155 |
| NAAA | N-acylethanolamine acid amidase | SEQ ID NOS: 9156- |
| | | 9161 |
| NAGA | N-acetylgalactosaminidase, alpha- | SEQ ID NOS: 9162- |
| | | 9164 |
| NAGLU | N-acetylglucosaminidase, alpha | SEQ ID NOS: 9165- |
| | | 9169 |
| NAGS | N-acetylglutamate synthase | SEQ ID NOS: 9170- |
| | | 9171 |
| NAPSA | Napsin A aspartic peptidase | SEQ ID NOS: 9172- |
| | | 9174 |
| NBL1 | Neuroblastoma 1, DAN family BMP | SEQ ID NOS: 9180- |
| | antagonist | 9193 |
| NCAM1 | Neural cell adhesion molecule 1 | SEQ ID NOS: 9194- |
| | | 9213 |
| NCAN | Neurocan | SEQ ID NOS: 9214- |
| | | 9215 |
| NCBP2-AS2 | NCBP2 antisense RNA 2 (head to head) | SEQ ID NO: 9216 |
| NCSTN | Nicastrin | SEQ ID NOS: 9217- |
| | | 9226 |
| NDNF | Neuron-derived neurotrophic factor | SEQ ID NOS: 9227- |
| | | 9229 |
| NDP | Norrie disease (pseudoglioma) | SEQ ID NOS: 9230- |
| | | 9232 |
| NDUFA10 | NADH dehydrogenase (ubiquinone) 1 alpha | SEQ ID NOS: 9233- |
| | subcomplex, 10, 42 kDa | 9242 |
| NDUFB5 | NADH dehydrogenase (ubiquinone) 1 beta | SEQ ID NOS: 9243- |
| | subcomplex, 5, 16 kDa | 9251 |
| NDUFS8 | NADH dehydrogenase (ubiquinone) Fe—S | SEQ ID NOS: 9252- |
| | protein 8, 23 kDa (NADH-coenzyme Q | 9261 |
| | reductase) | |
| NDUFV1 | NADH dehydrogenase (ubiquinone) | SEQ ID NOS: 9262- |
| | flavoprotein 1, 51 kDa | 9275 |
| NECAB3 | N-terminal EF-hand calcium binding | SEQ ID NOS: 9276- |
| | protein 3 | 9285 |
| NELL1 | Neural EGFL like 1 | SEQ ID NOS: 9289- |
| | | 9292 |
| NELL2 | Neural EGFL like 2 | SEQ ID NOS: 9293- |
| | | 9307 |
| NENF | Neudesin neurotrophic factor | SEQ ID NO: 9308 |
| NETO1 | Neuropilin (NRP) and tolloid (TLL)-like 1 | SEQ ID NOS: 9309- |
| | | 9312 |
| NFASC | Neurofascin | SEQ ID NOS: 9313- |
| | | 9327 |
| NFE2L1 | Nuclear factor, erythroid 2-like 1 | SEQ ID NOS: 9328- |
| | | 9346 |
| NFE2L3 | Nuclear factor, erythroid 2-like 3 | SEQ ID NOS: 9347- |
| | | 9348 |
| NGEF | Neuronal guanine nucleotide exchange | SEQ ID NOS: 9349- |
| | factor | 9354 |
| NGF | Nerve growth factor (beta polypeptide) | SEQ ID NO: 9355 |
| NGLY1 | N-glycanase 1 | SEQ ID NOS: 9356- |
| | | 9362 |
| NGRN | Neugrin, neurite outgrowth associated | SEQ ID NOS: 9363- |
| | | 9364 |
| NHLRC3 | NHL repeat containing 3 | SEQ ID NOS: 9365- |
| | | 9367 |
| NIDI | Nidogen 1 | SEQ ID NOS: 9368- |
| | | 9369 |
| NID2 | Nidogen 2 (osteonidogen) | SEQ ID NOS: 9370- |
| | | 9372 |
| NKG7 | Natural killer cell granule protein 7 | SEQ ID NOS: 9373- |
| | | 9377 |
| NLGN3 | Neuroligin 3 | SEQ ID NOS: 9378- |
| | | 9382 |
| NLGN4Y | Neuroligin 4, Y-linked | SEQ ID NOS: 9383- |
| | | 9389 |
| NLRP5 | NLR family, pyrin domain containing 5 | SEQ ID NOS: 9390- |
| | | 9392 |
| NMB | Neuromedin B | SEQ ID NOS: 9393- |
| | | 9394 |
| NME1 | NME/NM23 nucleoside diphosphate kinase | SEQ ID NOS: 9395- |
| | 1 | 9401 |
| NME1-NME2 | NME1-NME2 readthrough | SEQ ID NOS: 9402- |
| | | 9404 |
| NME3 | NME/NM23 nucleoside diphosphate kinase | SEQ ID NOS: 9405- |
| | 3 | 9409 |
| NMS | Neuromedin S | SEQ ID NO: 9410 |
| NMU | Neuromedin U | SEQ ID NOS: 9411- |
| | | 9414 |
| NOA1 | Nitric oxide associated 1 | SEQ ID NO: 9415 |
| NODAL | Nodal growth differentiation factor | SEQ ID NOS: 9416- |
| | | 9417 |
| NOG | Noggin | SEQ ID NO: 9418 |
| NOMO3 | NODAL modulator 3 | SEQ ID NOS: 9419- |
| | | 9425 |
| NOS1AP | Nitric oxide synthase 1 (neuronal) adaptor | SEQ ID NOS: 9426- |
| | protein | 9430 |
| NOTCH3 | Notch 3 | SEQ ID NOS: 9431- |
| | | 9434 |
| NOTUM | Notum pectinacetylesterase homolog | SEQ ID NOS: 9435- |
| | (Drosophila) | 9437 |
| NOV | Nephroblastoma overexpressed | SEQ ID NO: 9438 |
| NPB | Neuropeptide B | SEQ ID NOS: 9439- |
| | | 9440 |
| NPC2 | Niemann-Pick disease, type C2 | SEQ ID NOS: 9441- |
| | | 9449 |
| NPFF | Neuropeptide FF-amide peptide precursor | SEQ ID NO: 9450 |
| NPFFR2 | Neuropeptide FF receptor 2 | SEQ ID NOS: 9451- |
| | | 9454 |
| NPHS1 | Nephrosis 1, congenital, Finnish type | SEQ ID NOS: 9455- |
| | (nephrin) | 9456 |
| NPNT | Nephronectin | SEQ ID NOS: 9457- |
| | | 9467 |
| NPPA | Natriuretic peptide A | SEQ ID NOS: 9468- |
| | | 9470 |
| NPPB | Natriuretic peptide B | SEQ ID NO: 9471 |
| NPPC | Natriuretic peptide C | SEQ ID NOS: 9472- |
| | | 9473 |
| NPS | Neuropeptide S | SEQ ID NO: 9474 |
| NPTX1 | Neuronal pentraxin I | SEQ ID NO: 9475 |
| NPTX2 | Neuronal pentraxin II | SEQ ID NO: 9476 |
| NPTXR | Neuronal pentraxin receptor | SEQ ID NOS: 9477- |
| | | 9478 |
| NPVF | Neuropeptide VF precursor | SEQ ID NO: 9479 |
| NPW | Neuropeptide W | SEQ ID NOS: 9480- |
| | | 9482 |
| NPY | Neuropeptide Y | SEQ ID NOS: 9483- |
| | | 9485 |
| NQO2 | NAD(P)H dehydrogenase, quinone 2 | SEQ ID NOS: 9486- |
| | | 9494 |
| NRCAM | Neuronal cell adhesion molecule | SEQ ID NOS: 9495- |
| | | 9507 |
| NRG1 | Neuregulin 1 | SEQ ID NOS: 9508- |
| | | 9525 |
| NRN1L | Neuritin 1-like | SEQ ID NOS: 9526- |
| | | 9528 |
| NRP1 | Neuropilin 1 | SEQ ID NOS: 9529- |
| | | 9542 |
| NRP2 | Neuropilin 2 | SEQ ID NOS: 9543- |
| | | 9549 |
| NRTN | Neurturin | SEQ ID NO: 9550 |
| NRXN1 | Neurexin 1 | SEQ ID NOS: 9551- |
| | | 9581 |
| NRXN2 | Neurexin 2 | SEQ ID NOS: 9582- |
| | | 9590 |
| NT5C3A | 5′-nucleotidase, cytosolic IIIA | SEQ ID NOS: 9591- |
| | | 9601 |
| NT5DC3 | 5′-nucleotidase domain containing 3 | SEQ ID NOS: 9602- |
| | | 9604 |
| NT5E | 5′-nucleotidase, ecto (CD73) | SEQ ID NOS: 9605- |
| | | 9609 |
| NTF3 | Neurotrophin 3 | SEQ ID NOS: 9610- |
| | | 9611 |
| NTF4 | Neurotrophin 4 | SEQ ID NOS: 9612- |
| | | 9613 |
| NTM | Neurotrimin | SEQ ID NOS: 9614- |
| | | 9623 |
| NTN1 | Netrin 1 | SEQ ID NOS: 9624- |
| | | 9625 |
| NTN3 | Netrin 3 | SEQ ID NO: 9626 |
| NTN4 | Netrin 4 | SEQ ID NOS: 9627- |
| | | 9631 |
| NTN5 | Netrin 5 | SEQ ID NOS: 9632- |
| | | 9633 |
| NTNG1 | Netrin G1 | SEQ ID NOS: 9634- |
| | | 9640 |
| NTNG2 | Netrin G2 | SEQ ID NOS: 9641- |
| | | 9642 |
| NTS | Neurotensin | SEQ ID NOS: 9643- |
| | | 9644 |
| NUBPL | Nucleotide binding protein-like | SEQ ID NOS: 9645- |
| | | 9651 |
| NUCB1 | Nucleobindin 1 | SEQ ID NOS: 9652- |
| | | 9658 |
| NUCB2 | Nucleobindin 2 | SEQ ID NOS: 9659- |
| | | 9674 |
| NUDT19 | Nudix (nucleoside diphosphate linked | SEQ ID NO: 9675 |
| | moiety X)-type motif 19 | |
| NUDT9 | Nudix (nucleoside diphosphate linked | SEQ ID NOS: 9676- |
| | moiety X)-type motif 9 | 9680 |
| NUP155 | Nucleoporin 155 kDa | SEQ ID NOS: 9681- |
| | | 9684 |
| NUP214 | Nucleoporin 214 kDa | SEQ ID NOS: 9685- |
| | | 9696 |
| NUP85 | Nucleoporin 85 kDa | SEQ ID NOS: 9697- |
| | | 9711 |
| NXPE3 | Neurexophilin and PC-esterase domain | SEQ ID NOS: 9712- |
| | family, member 3 | 9716 |
| NXPE4 | Neurexophilin and PC-esterase domain | SEQ ID NOS: 9717- |
| | family, member 4 | 9718 |
| NXPH1 | Neurexophilin 1 | SEQ ID NOS: 9719- |
| | | 9722 |
| NXPH2 | Neurexophilin 2 | SEQ ID NO: 9723 |
| NXPH3 | Neurexophilin 3 | SEQ ID NOS: 9724- |
| | | 9725 |
| NXPH4 | Neurexophilin 4 | SEQ ID NOS: 9726- |
| | | 9727 |
| NYX | Nyctalopin | SEQ ID NOS: 9728- |
| | | 9729 |
| OAF | Out at first homolog | SEQ ID NOS: 9730- |
| | | 9731 |
| OBP2A | Odorant binding protein 2A | SEQ ID NOS: 9732- |
| | | 9738 |
| OBP2B | Odorant binding protein 2B | SEQ ID NOS: 9739- |
| | | 9742 |
| OC90 | Otoconin 90 | SEQ ID NO: 9743 |
| OCLN | Occludin | SEQ ID NOS: 9744- |
| | | 9746 |
| ODAM | Odontogenic, ameloblast asssociated | SEQ ID NOS: 9747- |
| | | 9750 |
| OGG1 | 8-oxoguanine DNA glvcosylase | SEQ ID NOS: 9755- |
| | | 9768 |
| OGN | Osteoglycin | SEQ ID NOS: 9769- |
| | | 9771 |
| OIT3 | Oncoprotein induced transcript 3 | SEQ ID NOS: 9772- |
| | | 9773 |
| OLFM1 | Olfactomedin 1 | SEQ ID NOS: 9774- |
| | | 9784 |
| OLFM2 | Olfactomedin 2 | SEQ ID NOS: 9785- |
| | | 9788 |
| OLFM3 | Olfactomedin 3 | SEQ ID NOS: 9789- |
| | | 9791 |
| OLFM4 | Olfactomedin 4 | SEQ ID NO: 9792 |
| OLFML1 | Olfactomedin-like 1 | SEQ ID NOS: 9793- |
| | | 9796 |
| OLFML2A | Olfactomedin-like 2A | SEQ ID NOS: 9797- |
| | | 9799 |
| OLFML2B | Olfactomedin-like 2B | SEQ ID NOS: 9800- |
| | | 9804 |
| OLFML3 | Olfactomedin-like 3 | SEQ ID NOS: 9805- |
| | | 9807 |
| OMD | Osteomodulin | SEQ ID NO: 9808 |
| OMG | Oligodendrocyte myelin glycoprotein | SEQ ID NO: 9809 |
| OOSP2 | Oocyte secreted protein 2 | SEQ ID NOS: 9810- |
| | | 9811 |
| OPCML | Opioid binding protein/cell adhesion | SEQ ID NOS: 9812- |
| | molecule-like | 9816 |
| OPTC | Opticin | SEQ ID NOS: 9818- |
| | | 9819 |
| ORAI1 | ORAI calcium release-activated calcium | SEQ ID NO: 9820 |
| | modulator 1 | |
| ORM1 | Orosomucoid 1 | SEQ ID NO: 9821 |
| ORM2 | Orosomucoid 2 | SEQ ID NO: 9822 |
| ORMDL2 | ORMDL sphingolipid biosynthesis | SEQ ID NOS: 9823- |
| | regulator 2 | 9826 |
| OS9 | Osteosarcoma amplified 9, endoplasmic | SEQ ID NOS: 9827- |
| | reticulum lectin | 9841 |
| OSCAR | Osteoclast associated, immunoglobulin-like | SEQ ID NOS: 9842- |
| | receptor | 9852 |
| OSM | Oncostatin M | SEQ ID NOS: 9853- |
| | | 9855 |
| OSMR | Oncostatin M receptor | SEQ ID NOS: 9856- |
| | | 9860 |
| OSTN | Osteocrin | SEQ ID NOS: 9861- |
| | | 9862 |
| OTOA | Otoancorin | SEQ ID NOS: 9863- |
| | | 9868 |
| OTOG | Otogelin | SEQ ID NOS: 9869- |
| | | 9871 |
| OTOGL | Otogelin-like | SEQ ID NOS: 9872- |
| | | 9878 |
| OTOL1 | Otolin 1 | SEQ ID NO: 9879 |
| OTOR | Otoraplin | SEQ ID NO: 9880 |
| OTOS | Otospiralin | SEQ ID NOS: 9881- |
| | | 9882 |
| OVCH1 | Ovochymase 1 | SEQ ID NOS: 9883- |
| | | 9885 |
| OVCH2 | Ovochymase 2 (gene/pseudogene) | SEQ ID NOS: 9886- |
| | | 9887 |
| OVGP1 | Oviductal glycoprotein 1, 120 kDa | SEQ ID NO: 9888 |
| OXCT1 | 3-oxoacid CoA transferase 1 | SEQ ID NOS: 9889- |
| | | 9892 |
| OXCT2 | 3-oxoacid CoA transferase 2 | SEQ ID NO: 9893 |
| OXNAD1 | Oxidoreductase NAD-binding domain | SEQ ID NOS: 9894- |
| | containing 1 | 9900 |
| OXT | Oxytocin/neurophysin I prepropeptide | SEQ ID NO: 9901 |
| P3H1 | Prolyl 3-hydroxylase 1 | SEQ ID NOS: 9902- |
| | | 9906 |
| P3H2 | Prolyl 3-hydroxylase 2 | SEQ ID NOS: 9907- |
| | | 9910 |
| P3H3 | Prolyl 3-hydroxylase 3 | SEQ ID NO: 9911 |
| P3H4 | Prolyl 3-hydroxylase family member 4 | SEQ ID NOS: 9912- |
| | (non-enzymatic) | 9916 |
| P4HA1 | Prolyl 4-hydroxylase, alpha polypeptide I | SEQ ID NOS: 9917- |
| | | 9921 |
| P4HA2 | Prolyl 4-hydroxylase, alpha polypeptide II | SEQ ID NOS: 9922- |
| | | 9936 |
| P4HA3 | Prolyl 4-hydroxylase, alpha polypeptide III | SEQ ID NOS: 9937- |
| | | 9941 |
| P4HB | Prolyl 4-hydroxylase, beta polypeptide | SEQ ID NOS: 9942- |
| | | 9953 |
| PAEP | Progestagen-associated endometrial protein | SEQ ID NOS: 9954- |
| | | 9962 |
| PAM | Peptidylglycine alpha-amidating | SEQ ID NOS: 9963- |
| | monooxygenase | 9976 |
| PAMR1 | Peptidase domain containing associated | SEQ ID NOS: 9977- |
| | with muscle regeneration 1 | 9983 |
| PAPL | Iron/zinc purple acid phosphatase-like | SEQ ID NOS: 159-162 |
| | protein | |
| PAPLN | Papilin, proteoglycan-like sulfated | SEQ ID NOS: 9984- |
| | glycoprotein | 9991 |
| PAPPA | Pregnancy-associated plasma protein A, | SEQ ID NO: 9992 |
| | pappalysin 1 | |
| PAPPA2 | Pappalysin 2 | SEQ ID NOS: 9993- |
| | | 9994 |
| PARP15 | Poly (ADP-ribose) polymerase family, | SEQ ID NOS: 9995- |
| | member 15 | 9998 |
| PARVB | Parvin, beta | SEQ ID NOS: 9999- |
| | | 10003 |
| PATE1 | Prostate and testis expressed 1 | SEQ ID NOS: 10004- |
| | | 10005 |
| PATE2 | Prostate and testis expressed 2 | SEQ ID NOS: 10006- |
| | | 10007 |
| PATE3 | Prostate and testis expressed 3 | SEQ ID NO: 10008 |
| PATE4 | Prostate and testis expressed 4 | SEQ ID NOS: 10009- |
| | | 10010 |
| PATL2 | Protein associated with topoisomerase II | SEQ ID NOS: 10011- |
| | homolog 2 (yeast) | 10016 |
| PAX2 | Paired box 2 | SEQ ID NOS: 10017- |
| | | 10022 |
| PAX4 | Paired box 4 | SEQ ID NOS: 10023- |
| | | 10029 |
| PCCB | Propionyl CoA carboxylase, beta | SEQ ID NOS: 10030- |
| | polypeptide | 10044 |
| PCDH1 | Protocadherin 1 | SEQ ID NOS: 10045- |
| | | 10050 |
| PCDH12 | Protocadherin 12 | SEQ ID NOS: 10051- |
| | | 10052 |
| PCDH15 | Protocadherin-related 15 | SEQ ID NOS: 10053- |
| | | 10086 |
| PCDHA1 | Protocadherin alpha 1 | SEQ ID NOS: 10087- |
| | | 10089 |
| PCDHA10 | Protocadherin alpha 10 | SEQ ID NOS: 10090- |
| | | 10092 |
| PCDHA11 | Protocadherin alpha 11 | SEQ ID NOS: 10093- |
| | | 10095 |
| PCDHA6 | Protocadherin alpha 6 | SEQ ID NOS: 10096- |
| | | 10098 |
| PCDHB12 | Protocadherin beta 12 | SEQ ID NOS: 10099- |
| | | 10101 |
| PCDHGA11 | Protocadherin gamma subfamily A, 11 | SEQ ID NOS: 10102- |
| | | 10104 |
| PCF11 | PCF11 cleavage and polyadenylation factor | SEQ ID NOS: 10105- |
| | subunit | 10109 |
| PCOLCE | Procollagen C-endopeptidase enhancer | SEQ ID NO: 10110 |
| PCOLCE2 | Procollagen C-endopeptidase enhancer 2 | SEQ ID NOS: 10111- |
| | | 10114 |
| PCSK1 | Proprotein convertase subtilisin/kexin type | SEQ ID NOS: 10115- |
| | 1 | 10117 |
| PCSK1N | Proprotein convertase subtilisin/kexin type | SEQ ID NO: 10118 |
| | 1 inhibitor | |
| PCSK2 | Proprotein convertase subtilisin/kexin type | SEQ ID NOS: 10119- |
| | 2 | 10121 |
| PCSK4 | Proprotein convertase subtilisin/kexin type | SEQ ID NOS: 10122- |
| | 4 | 10124 |
| PCSK5 | Proprotein convertase subtilisin/kexin type | SEQ ID NOS: 10125- |
| | 5 | 10129 |
| PCSK9 | Proprotein convertase subtilisin/kexin type | SEQ ID NO: 10130 |
| | 9 | |
| PCYOX1 | Prenylcysteine oxidase 1 | SEQ ID NOS: 10131- |
| | | 10135 |
| PCYOX1L | Prenylcysteine oxidase 1 like | SEQ ID NOS: 10136- |
| | | 10140 |
| PDDC1 | Parkinson disease 7 domain containing 1 | SEQ ID NOS: 5802- |
| | | 5810 |
| PDE11A | Phosphodiesterase 11A | SEQ ID NOS: 10141- |
| | | 10146 |
| PDE2A | Phosphodiesterase 2A, cGMP-stimulated | SEQ ID NOS: 10147- |
| | | 10168 |
| PDE7A | Phosphodiesterase 7A | SEQ ID NOS: 10169- |
| | | 10172 |
| PDF | Peptide deformylase (mitochondrial) | SEQ ID NO: 10173 |
| PDGFA | Platelet-derived growth factor alpha | SEQ ID NOS: 10174- |
| | polypeptide | 10177 |
| PDGFB | Platelet-derived growth factor beta | SEQ ID NOS: 10178- |
| | polypeptide | 10181 |
| PDGFC | Platelet derived growth factor C | SEQ ID NOS: 10182- |
| | | 10185 |
| PDGFD | Platelet derived growth factor D | SEQ ID NOS: 10186- |
| | | 10188 |
| PDGFRA | Platelet-derived growth factor receptor, | SEQ ID NOS: 10189- |
| | alpha polypeptide | 10195 |
| PDGFRB | Platelet-derived growth factor receptor, beta | SEQ ID NOS: 10196- |
| | polypeptide | 10199 |
| PDGFRL | Platelet-derived growth factor receptor-like | SEQ ID NOS: 10200- |
| | | 10201 |
| PDHA1 | Pyruvate dehydrogenase (lipoamide) alpha | SEQ ID NOS: 10202- |
| | 1 | 10210 |
| PDIA2 | Protein disulfide isomerase family A, | SEQ ID NOS: 10211- |
| | member 2 | 10214 |
| PDIA3 | Protein disulfide isomerase family A, | SEQ ID NOS: 10215- |
| | member 3 | 10218 |
| PDIA4 | Protein disulfide isomerase family A, | SEQ ID NOS: 10219- |
| | member 4 | 10220 |
| PDIA5 | Protein disulfide isomerase family A, | SEQ ID NOS: 10221- |
| | member 5 | 10224 |
| PDIA6 | Protein disulfide isomerase family A, | SEQ ID NOS: 10225- |
| | member 6 | 10231 |
| PDILT | Protein disulfide isomerase-like, testis | SEQ ID NOS: 10232- |
| | expressed | 10233 |
| PDYN | Prodynorphin | SEQ ID NOS: 10234- |
| | | 10236 |
| PDZD8 | PDZ domain containing 8 | SEQ ID NO: 10237 |
| PDZRN4 | PDZ domain containing ring finger 4 | SEQ ID NOS: 10238- |
| | | 10240 |
| PEAR1 | Platelet endothelial aggregation receptor 1 | SEQ ID NOS: 10241- |
| | | 10244 |
| PEBP4 | Phosphatidylethanolamine-binding protein 4 | SEQ ID NOS: 10245- |
| | | 10246 |
| PECAM1 | Platelet/endothelial cell adhesion molecule | SEQ ID NOS: 10247- |
| | 1 | 10250 |
| PENK | Proenkephalin | SEQ ID NOS: 10251- |
| | | 10256 |
| PET117 | PET117 homolog | SEQ ID NO: 10257 |
| PF4 | Platelet factor 4 | SEQ ID NO: 10258 |
| PF4V1 | Platelet factor 4 variant 1 | SEQ ID NO: 10259 |
| PFKP | Phosphofructokinase, platelet | SEQ ID NOS: 10260- |
| | | 10268 |
| PFN1 | Profilin 1 | SEQ ID NOS: 10269- |
| | | 10271 |
| PGA3 | Pepsinogen 3, group I (pepsinogen A) | SEQ ID NOS: 10272- |
| | | 10275 |
| PGA4 | Pepsinogen 4, group I (pepsinogen A) | SEQ ID NOS: 10276- |
| | | 10278 |
| PGA5 | Pepsinogen 5, group I (pepsinogen A) | SEQ ID NOS: 10279- |
| | | 10281 |
| PGAM5 | PGAM family member 5, serine/threonine | SEQ ID NOS: 10282- |
| | protein phosphatase, mitochondrial | 10285 |
| PGAP3 | Post-GPI attachment to proteins 3 | SEQ ID NOS: 10286- |
| | | 10293 |
| PGC | Progastricsin (pepsinogen C) | SEQ ID NOS: 10294- |
| | | 10297 |
| PGF | Placental growth factor | SEQ ID NOS: 10298- |
| | | 10301 |
| PGLYRP1 | Peptidoglycan recognition protein 1 | SEQ ID NO: 10302 |
| PGLYRP2 | Peptidoglycan recognition protein 2 | SEQ ID NOS: 10303- |
| | | 10306 |
| PGLYRP3 | Peptidoglycan recognition protein 3 | SEQ ID NO: 10307 |
| PGLYRP4 | Peptidoglycan recognition protein 4 | SEQ ID NOS: 10308- |
| | | 10309 |
| PHACTR1 | Phosphatase and actin regulator 1 | SEQ ID NOS: 10310- |
| | | 10316 |
| PHB | Prohibitin | SEQ ID NOS: 10317- |
| | | 10325 |
| PI15 | Peptidase inhibitor 15 | SEQ ID NOS: 10326- |
| | | 10327 |
| PI3 | Peptidase inhibitor 3, skin-derived | SEQ ID NO: 10328 |
| PIANP | PILR alpha associated neural protein | SEQ ID NOS: 10329- |
| | | 10334 |
| PIGK | Phosphatidylinositol glycan anchor | SEQ ID NOS: 10335- |
| | biosynthesis, class K | 10338 |
| PIGL | Phosphatidylinositol glycan anchor | SEQ ID NOS: 10339- |
| | biosynthesis, class L | 10346 |
| PIGT | Phosphatidylinositol glycan anchor | SEQ ID NOS: 10347- |
| | biosynthesis, class T | 10400 |
| PIGZ | Phosphatidylinositol glycan anchor | SEQ ID NOS: 10401- |
| | biosynthesis, class Z | 10403 |
| PIK3AP1 | Phosphoinositide-3-kinase adaptor protein 1 | SEQ ID NOS: 10404- |
| | | 10406 |
| PIK3IP1 | Phosphoinositide-3-kinase interacting | SEQ ID NOS: 10407- |
| | protein 1 | 10410 |
| PILRA | Paired immunoglobin-like type 2 receptor | SEQ ID NOS: 10411- |
| | alpha | 10415 |
| PILRB | Paired immunoglobin-like type 2 receptor | SEQ ID NOS: 10416- |
| | beta | 10427 |
| PINLYP | Phospholipase A2 inhibitor and | SEQ ID NOS: 10428- |
| | LY6/PLAUR domain containing | 10432 |
| PIP | Prolactin-induced protein | SEQ ID NO: 10433 |
| PIWIL4 | Piwi-like RNA-mediated gene silencing 4 | SEQ ID NOS: 10434- |
| | | 10438 |
| PKDCC | Protein kinase domain containing, | SEQ ID NOS: 10439- |
| | cytoplasmic | 10440 |
| PKHD1 | Polycystic kidney and hepatic disease 1 | SEQ ID NOS: 10441- |
| | (autosomal recessive) | 10442 |
| PLA1A | Phospholipase A1 member A | SEQ ID NOS: 10443- |
| | | 10447 |
| PLA2G10 | Phospholipase A2, group X | SEQ ID NOS: 10448- |
| | | 10449 |
| PLA2G12A | Phospholipase A2, group XIIA | SEQ ID NOS: 10450- |
| | | 10452 |
| PLA2G12B | Phospholipase A2, group XIIB | SEQ ID NO: 10453 |
| PLA2G15 | Phospholipase A2, group XV | SEQ ID NOS: 10454- |
| | | 10461 |
| PLA2G1B | Phospholipase A2, group IB (pancreas) | SEQ ID NOS: 10462- |
| | | 10464 |
| PLA2G2A | Phospholipase A2, group IIA (platelets, | SEQ ID NOS: 10465- |
| | synovial fluid) | 10466 |
| PLA2G2C | Phospholipase A2, group IIC | SEQ ID NOS: 10467- |
| | | 10468 |
| PLA2G2D | Phospholipase A2, group IID | SEQ ID NOS: 10469- |
| | | 10470 |
| PLA2G2E | Phospholipase A2, group IIE | SEQ ID NO: 10471 |
| PLA2G3 | Phospholipase A2, group III | SEQ ID NO: 10472 |
| PLA2G5 | Phospholipase A2, group V | SEQ ID NO: 10473 |
| PLA2G7 | Phospholipase A2, group VII (platelet- | SEQ ID NOS: 10474- |
| | activating factor acetylhydrolase, plasma) | 10475 |
| PLA2R1 | Phospholipase A2 receptor 1, 180 kDa | SEQ ID NOS: 10476- |
| | | 10477 |
| PLAC1 | Placenta-specific 1 | SEQ ID NO: 10478 |
| PLAC9 | Placenta-specific 9 | SEQ ID NOS: 10479- |
| | | 10481 |
| PLAT | Plasminogen activator, tissue | SEQ ID NOS: 10482- |
| | | 10490 |
| PLAU | Plasminogen activator, urokinase | SEQ ID NOS: 10491- |
| | | 10493 |
| PLAUR | Plasminogen activator, urokinase receptor | SEQ ID NOS: 10494- |
| | | 10505 |
| PLBD1 | Phospholipase B domain containing 1 | SEQ ID NOS: 10506- |
| | | 10508 |
| PLBD2 | Phospholipase B domain containing 2 | SEQ ID NOS: 10509- |
| | | 10511 |
| PLG | Plasminogen | SEQ ID NOS: 10512- |
| | | 10514 |
| PLGLB1 | Plasminogen-like B1 | SEQ ID NOS: 10515- |
| | | 10518 |
| PLGLB2 | Plasminogen-like B2 | SEQ ID NOS: 10519- |
| | | 10520 |
| PLOD1 | Procollagen-lysine, 2-oxoglutarate 5- | SEQ ID NOS: 10521- |
| | dioxygenase 1 | 10523 |
| PLOD2 | Procollagen-lysine, 2-oxoglutarate 5- | SEQ ID NOS: 10524- |
| | dioxygenase 2 | 10529 |
| PLOD3 | Procollagen-lysine, 2-oxoglutarate 5- | SEQ ID NOS: 10530- |
| | dioxygenase 3 | 10536 |
| PLTP | Phospholipid transfer protein | SEQ ID NOS: 10537- |
| | | 10541 |
| PLXNA4 | Plexin A4 | SEQ ID NOS: 10542- |
| | | 10545 |
| PLXNB2 | Plexin B2 | SEQ ID NOS: 10546- |
| | | 10554 |
| PM20D1 | Peptidase M20 domain containing 1 | SEQ ID NO: 10555 |
| PMCH | Pro-melanin-concentrating hormone | SEQ ID NO: 10556 |
| PMEL | Premelanosome protein | SEQ ID NOS: 10557- |
| | | 10568 |
| PMEPA1 | Prostate transmembrane protein, androgen | SEQ ID NOS: 10569- |
| | induced 1 | 10575 |
| PNLIP | Pancreatic lipase | SEQ ID NO: 10576 |
| PNLIPRP1 | Pancreatic lipase-related protein 1 | SEQ ID NOS: 10577- |
| | | 10585 |
| PNLIPRP3 | Pancreatic lipase-related protein 3 | SEQ ID NO: 10586 |
| PNOC | Prepronociceptin | SEQ ID NOS: 10587- |
| | | 10589 |
| PNP | Purine nucleoside phosphorylase | SEQ ID NOS: 10590- |
| | | 10593 |
| PNPLA4 | Patatin-like phospholipase domain | SEQ ID NOS: 10594- |
| | containing 4 | 10597 |
| PODNL1 | Podocan-like 1 | SEQ ID NOS: 10598- |
| | | 10609 |
| POFUT1 | Protein O-fucosyltransferase 1 | SEQ ID NOS: 10610- |
| | | 10611 |
| POFUT2 | Protein O-fucosyltransferase 2 | SEQ ID NOS: 10612- |
| | | 10617 |
| POGLUT1 | Protein O-glucosyltransferase 1 | SEQ ID NOS: 10618- |
| | | 10622 |
| POLL | Polymerase (DNA directed), lambda | SEQ ID NOS: 10623- |
| | | 10635 |
| POMC | Proopiomelanocortin | SEQ ID NOS: 10636- |
| | | 10640 |
| POMGNT2 | Protein O-linked mannose N- | SEQ ID NOS: 10641- |
| | acetylglucosaminyltransferase 2 (beta 1,4-) | 10642 |
| PON1 | Paraoxonase 1 | SEQ ID NOS: 10643- |
| | | 10644 |
| PON2 | Paraoxonase 2 | SEQ ID NOS: 10645- |
| | | 10657 |
| PON3 | Paraoxonase 3 | SEQ ID NOS: 10658- |
| | | 10663 |
| POSTN | Periostin, osteoblast specific factor | SEQ ID NOS: 10664- |
| | | 10669 |
| PPBP | Pro-platelet basic protein (chemokine (C-X- | SEQ ID NO: 10670 |
| | C motif) ligand 7) | |
| PPIB | Peptidylprolyl isomerase B (cyclophilin B) | SEQ ID NO: 10671 |
| PPIC | Peptidylprolyl isomerase C (cyclophilin C) | SEQ ID NO: 10672 |
| PPOX | Protoporphyrinogen oxidase | SEQ ID NOS: 10673- |
| | | 10683 |
| PPP1CA | Protein phosphatase 1, catalytic subunit, | SEQ ID NOS: 10684- |
| | alpha isozyme | 10689 |
| PPT1 | Palmitoyl-protein thioesterase 1 | SEQ ID NOS: 10690- |
| | | 10706 |
| PPT2 | Palmitoyl-protein thioesterase 2 | SEQ ID NOS: 10707- |
| | | 10714 |
| PPY | Pancreatic polypeptide | SEQ ID NOS: 10715- |
| | | 10719 |
| PRAC2 | Prostate cancer susceptibility candidate 2 | SEQ ID NOS: 10720- |
| | | 10721 |
| PRADC1 | Protease-associated domain containing 1 | SEQ ID NO: 10722 |
| PRAP1 | Proline-rich acidic protein 1 | SEQ ID NOS: 10723- |
| | | 10724 |
| PRB1 | Proline-rich protein BstNI subfamily 1 | SEQ ID NOS: 10725- |
| | | 10728 |
| PRB2 | Proline-rich protein BstNI subfamily 2 | SEQ ID NOS: 10729- |
| | | 10730 |
| PRB3 | Proline-rich protein BstNI subfamily 3 | SEQ ID NOS: 10731- |
| | | 10732 |
| PRB4 | Proline-rich protein BstNI subfamily 4 | SEQ ID NOS: 10733- |
| | | 10736 |
| PRCD | Progressive rod-cone degeneration | SEQ ID NOS: 10737- |
| | | 10738 |
| PRCP | Prolylcarboxypeptidase (angiotensinase C) | SEQ ID NOS: 10739- |
| | | 10750 |
| PRDM12 | PR domain containing 12 | SEQ ID NO: 10751 |
| PRDX4 | Peroxiredoxin 4 | SEQ ID NOS: 10752- |
| | | 10755 |
| PRELP | Proline/arginine-rich end leucine-rich repeat | SEQ ID NO: 10756 |
| | protein | |
| PRF1 | Perforin 1 (pore forming protein) | SEQ ID NOS: 10757- |
| | | 10759 |
| PRG2 | Proteoglycan 2, bone marrow (natural killer | SEQ ID NOS: 10760- |
| | cell activator, eosinophil granule major | 10762 |
| | basic protein) | |
| PRG3 | Proteoglycan 3 | SEQ ID NO: 10763 |
| PRG4 | Proteoglycan 4 | SEQ ID NOS: 10764- |
| | | 10769 |
| PRH1 | Proline-rich protein HaeIII subfamily 1 | SEQ ID NOS: 10770- |
| | | 10772 |
| PRH2 | Proline-rich protein HaeIII subfamily 2 | SEQ ID NOS: 10773- |
| | | 10774 |
| PRKAG1 | Protein kinase, AMP-activated, gamma 1 | SEQ ID NOS: 10775- |
| | non-catalytic subunit | 10789 |
| PRKCSH | Protein kinase C substrate 80K-H | SEQ ID NOS: 10790- |
| | | 10799 |
| PRKD1 | Protein kinase D1 | SEQ ID NOS: 10800- |
| | | 10805 |
| PRL | Prolactin | SEQ ID NOS: 10806- |
| | | 10808 |
| PRLH | Prolactin releasing hormone | SEQ ID NO: 10809 |
| PRLR | Prolactin receptor | SEQ ID NOS: 10810- |
| | | 10828 |
| PRNP | Prion protein | SEQ ID NOS: 10829- |
| | | 10832 |
| PRNT | Prion protein (testis specific) | SEQ ID NO: 10833 |
| PROC | Protein C (inactivator of coagulation factors | SEQ ID NOS: 10834- |
| | Va and VIIIa) | 10841 |
| PROK1 | Prokineticin 1 | SEQ ID NO: 10842 |
| PROK2 | Prokineticin 2 | SEQ ID NOS: 10843- |
| | | 10844 |
| PROL1 | Proline rich, lacrimal 1 | SEQ ID NO: 9817 |
| PROM1 | Prominin 1 | SEQ ID NOS: 10845- |
| | | 10856 |
| PROS1 | Protein S (alpha) | SEQ ID NOS: 10857- |
| | | 10860 |
| PROZ | Protein Z, vitamin K-dependent plasma | SEQ ID NOS: 10861- |
| | glycoprotein | 10862 |
| PRR27 | Proline rich 27 | SEQ ID NOS: 10863- |
| | | 10866 |
| PRR4 | Proline rich 4 (lacrimal) | SEQ ID NOS: 10867- |
| | | 10869 |
| PRRG2 | Proline rich Gla (G-carboxyglutamic acid) 2 | SEQ ID NOS: 10870- |
| | | 10872 |
| PRRT3 | Proline-rich transmembrane protein 3 | SEQ ID NOS: 10873- |
| | | 10875 |
| PRRT4 | Proline-rich transmembrane protein 4 | SEQ ID NOS: 10876- |
| | | 10882 |
| PRSS1 | Protease, serine, 1 (trypsin 1) | SEQ ID NOS: 10883- |
| | | 10886 |
| PRSS12 | Protease, serine, 12 (neurotrypsin, | SEQ ID NO: 10887 |
| | motopsin) | |
| PRSS16 | Protease, serine, 16 (thymus) | SEQ ID NOS: 10888- |
| | | 10895 |
| PRSS2 | Protease, serine, 2 (trypsin 2) | SEQ ID NOS: 10896- |
| | | 10899 |
| PRSS21 | Protease, serine, 21 (testisin) | SEQ ID NOS: 10900- |
| | | 10905 |
| PRSS22 | Protease, serine, 22 | SEQ ID NOS: 10906- |
| | | 10908 |
| PRSS23 | Protease, serine, 23 | SEQ ID NOS: 10909- |
| | | 10912 |
| PRSS27 | Protease, serine 27 | SEQ ID NOS: 10913- |
| | | 10915 |
| PRSS3 | Protease, serine, 3 | SEQ ID NOS: 10916- |
| | | 10920 |
| PRSS33 | Protease, serine, 33 | SEQ ID NOS: 10921- |
| | | 10924 |
| PRSS35 | Protease, serine, 35 | SEQ ID NO: 10925 |
| PRSS36 | Protease, serine, 36 | SEQ ID NOS: 10926- |
| | | 10929 |
| PRSS37 | Protease, serine, 37 | SEQ ID NOS: 10930- |
| | | 10933 |
| PRSS38 | Protease, serine, 38 | SEQ ID NO: 10934 |
| PRSS42 | Protease, serine, 42 | SEQ ID NOS: 10935- |
| | | 10936 |
| PRSS48 | Protease, serine, 48 | SEQ ID NOS: 10937- |
| | | 10938 |
| PRSS50 | Protease, serine, 50 | SEQ ID NO: 10939 |
| PRSS53 | Protease, serine, 53 | SEQ ID NO: 10940 |
| PRSS54 | Protease, serine, 54 | SEQ ID NOS: 10941- |
| | | 10945 |
| PRSS55 | Protease, serine, 55 | SEQ ID NOS: 10946- |
| | | 10948 |
| PRSS56 | Protease, serine, 56 | SEQ ID NOS: 10949- |
| | | 10950 |
| PRSS57 | Protease, serine, 57 | SEQ ID NOS: 10951- |
| | | 10952 |
| PRSS58 | Protease, serine, 58 | SEQ ID NOS: 10953- |
| | | 10954 |
| PRSS8 | Protease, serine, 8 | SEQ ID NOS: 10955- |
| | | 10958 |
| PRTG | Protogenin | SEQ ID NOS: 10959- |
| | | 10962 |
| PRTN3 | Proteinase 3 | SEQ ID NOS: 10963- |
| | | 10964 |
| PSAP | Prosaposin | SEQ ID NOS: 10965- |
| | | 10968 |
| PSAPL1 | Prosaposin-like 1 (gene/pseudogene) | SEQ ID NO: 10969 |
| PSG1 | Pregnancy specific beta-1-glycoprotein 1 | SEQ ID NOS: 10970- |
| | | 10977 |
| PSG11 | Pregnancy specific beta-1-glycoprotein 11 | SEQ ID NOS: 10978- |
| | | 10982 |
| PSG2 | Pregnancy specific beta-1-glycoprotein 2 | SEQ ID NOS: 10983- |
| | | 10984 |
| PSG3 | Pregnancy specific beta-1-glycoprotein 3 | SEQ ID NOS: 10985- |
| | | 10988 |
| PSG4 | Pregnancy specific beta-1-glycoprotein 4 | SEQ ID NOS: 10989- |
| | | 11000 |
| PSG5 | Pregnancy specific beta-1-glycoprotein 5 | SEQ ID NOS: 11001- |
| | | 11006 |
| PSG6 | Pregnancy specific beta-1-glycoprotein 6 | SEQ ID NOS: 11007- |
| | | 11012 |
| PSG7 | Pregnancy specific beta-1-glycoprotein 7 | SEQ ID NOS: 11013- |
| | (gene/pseudogene) | 11015 |
| PSG8 | Pregnancy specific beta-1-glycoprotein 8 | SEQ ID NOS: 11016- |
| | | 11020 |
| PSG9 | Pregnancy specific beta-1-glycoprotein 9 | SEQ ID NOS: 11021- |
| | | 11028 |
| PSMD1 | Proteasome 26S subunit, non-ATPase 1 | SEQ ID NOS: 11029- |
| | | 11036 |
| PSORS1C2 | Psoriasis susceptibility 1 candidate 2 | SEQ ID NO: 11037 |
| PSPN | Persephin | SEQ ID NOS: 11038- |
| | | 11039 |
| PTGDS | Prostaglandin D2 synthase 21 kDa (brain) | SEQ ID NOS: 11040- |
| | | 11044 |
| PTGIR | Prostaglandin I2 (prostacyclin) receptor (IP) | SEQ ID NOS: 11045- |
| | | 11049 |
| PTGS1 | Prostaglandin-endoperoxide synthase 1 | SEQ ID NOS: 11050- |
| | (prostaglandin G/H synthase and | 11058 |
| | cyclooxygenase) | |
| PTGS2 | Prostaglandin-endoperoxide synthase 2 | SEQ ID NOS: 11059- |
| | (prostaglandin G/H synthase and | 11060 |
| | cyclooxygenase) | |
| PTH | Parathyroid hormone | SEQ ID NOS: 11061- |
| | | 11062 |
| PTH2 | Parathyroid hormone 2 | SEQ ID NO: 11063 |
| PTHLH | Parathyroid hormone-like hormone | SEQ ID NOS: 11064- |
| | | 11072 |
| PTK7 | Protein tyrosine kinase 7 (inactive) | SEQ ID NOS: 11073- |
| | | 11088 |
| PTN | Pleiotrophin | SEQ ID NOS: 11089- |
| | | 11090 |
| PTPRA | Protein tyrosine phosphatase, receptor type, | SEQ ID NOS: 11091- |
| | A | 11098 |
| PTPRB | Protein tyrosine phosphatase, receptor type, | SEQ ID NOS: 11099- |
| | B | 11106 |
| PTPRC | Protein tyrosine phosphatase, receptor type, | SEQ ID NOS: 11107- |
| | C | 11117 |
| PTPRCAP | Protein tyrosine phosphatase, receptor type, | SEQ ID NO: 11118 |
| | C-associated protein | |
| PTPRD | Protein tyrosine phosphatase, receptor type, | SEQ ID NOS: 11119- |
| | D | 11130 |
| PTPRF | Protein tyrosine phosphatase, receptor type, | SEQ ID NOS: 11131- |
| | F | 11138 |
| PTPRJ | Protein tyrosine phosphatase, receptor type, | SEQ ID NOS: 11139- |
| | J | 11144 |
| PTPRO | Protein tyrosine phosphatase, receptor type, | SEQ ID NOS: 11145- |
| | O | 11153 |
| PTPRS | Protein tyrosine phosphatase, receptor type, | SEQ ID NOS: 11154- |
| | S | 11161 |
| PTTG1IP | Pituitary tumor-transforming 1 interacting | SEQ ID NOS: 11162- |
| | protein | 11165 |
| PTX3 | Pentraxin 3, long | SEQ ID NO: 11166 |
| PTX4 | Pentraxin 4, long | SEQ ID NOS: 11167- |
| | | 11169 |
| PVR | Poliovirus receptor | SEQ ID NOS: 11170- |
| | | 11175 |
| PVRL1 | Poliovirus receptor-related 1 (herpesvirus | SEQ ID NOS: 9286- |
| | entry mediator C) | 9288 |
| PXDN | Peroxidasin | SEQ ID NOS: 11176- |
| | | 11180 |
| PXDNL | Peroxidasin-like | SEQ ID NOS: 11181- |
| | | 11183 |
| PXYLP1 | 2-phosphoxylose phosphatase 1 | SEQ ID NOS: 11184- |
| | | 11196 |
| PYY | Peptide YY | SEQ ID NOS: 11197- |
| | | 11198 |
| PZP | Pregnancy-zone protein | SEQ ID NOS: 11199- |
| | | 11200 |
| QPCT | Glutaminyl-peptide cyclotransferase | SEQ ID NOS: 11201- |
| | | 11203 |
| QPRT | Quinolinate phosphoribosyltransferase | SEQ ID NOS: 11204- |
| | | 11205 |
| QRFP | Pyroglutamylated RFamide peptide | SEQ ID NOS: 11206- |
| | | 11207 |
| QSOX1 | Quiescin Q6 sulfhydryl oxidase 1 | SEQ ID NOS: 11208- |
| | | 11211 |
| R3HDML | R3H domain containing-like | SEQ ID NO: 11212 |
| RAB26 | RAB26, member RAS oncogene family | SEQ ID NOS: 11213- |
| | | 11216 |
| RAB36 | RAB36, member RAS oncogene family | SEQ ID NOS: 11217- |
| | | 11219 |
| RAB9B | RAB9B, member RAS oncogene family | SEQ ID NO: 11220 |
| RAET1E | Retinoic acid early transcript 1E | SEQ ID NOS: 11221- |
| | | 11226 |
| RAET1G | Retinoic acid early transcript 1G | SEQ ID NOS: 11227- |
| | | 11229 |
| RAMP2 | Receptor (G protein-coupled) activity | SEQ ID NOS: 11230- |
| | modifying protein 2 | 11234 |
| RAPGEF5 | Rap guanine nucleotide exchange factor | SEQ ID NOS: 11235- |
| | (GEF) 5 | 11241 |
| RARRES1 | Retinoic acid receptor responder (tazarotene | SEQ ID NOS: 11242- |
| | induced) 1 | 11243 |
| RARRES2 | Retinoic acid receptor responder (tazarotene | SEQ ID NOS: 11244- |
| | induced) 2 | 11247 |
| RASA2 | RAS p21 protein activator 2 | SEQ ID NOS: 11248- |
| | | 11250 |
| RBM3 | RNA binding motif (RNP1, RRM) protein 3 | SEQ ID NOS: 11251- |
| | | 11253 |
| RBP3 | Retinol binding protein 3, interstitial | SEQ ID NO: 11254 |
| RBP4 | Retinol binding protein 4, plasma | SEQ ID NOS: 11255- |
| | | 11258 |
| RCN1 | Reticulocalbin 1, EF-hand calcium binding | SEQ ID NOS: 11259- |
| | domain | 11262 |
| RCN2 | Reticulocalbin 2, EF-hand calcium binding | SEQ ID NOS: 11263- |
| | domain | 11266 |
| RCN3 | Reticulocalbin 3, EF-hand calcium binding | SEQ ID NOS: 11267- |
| | domain | 11270 |
| RCOR1 | REST corepressor 1 | SEQ ID NOS: 11271- |
| | | 11272 |
| RDH11 | Retinol dehydrogenase 11 (all-trans/9- | SEQ ID NOS: 11273- |
| | cis/11-cis) | 11280 |
| RDH12 | Retinol dehydrogenase 12 (all-trans/9- | SEQ ID NOS: 11281- |
| | cis/11-cis) | 11282 |
| RDH13 | Retinol dehydrogenase 13 (all-trans/9-cis) | SEQ ID NOS: 11283- |
| | | 11291 |
| RDH5 | Retinol dehydrogenase 5 (11-cis/9-cis) | SEQ ID NOS: 11292- |
| | | 11296 |
| RDH8 | Retinol dehydrogenase 8 (all-trans) | SEQ ID NOS: 11297- |
| | | 11298 |
| REG1A | Regenerating islet-derived 1 alpha | SEQ ID NO: 11299 |
| REG1B | Regenerating islet-derived 1 beta | SEQ ID NOS: 11300- |
| | | 11301 |
| REG3A | Regenerating islet-derived 3 alpha | SEQ ID NOS: 11302- |
| | | 11304 |
| REG3G | Regenerating islet-derived 3 gamma | SEQ ID NOS: 11305- |
| | | 11307 |
| REG4 | Regenerating islet-derived family, member | SEQ ID NOS: 11308- |
| | 4 | 11311 |
| RELN | Reelin | SEQ ID NOS: 11312- |
| | | 11315 |
| RELT | RELT tumor necrosis factor receptor | SEQ ID NOS: 11316- |
| | | 11319 |
| REN | Renin | SEQ ID NOS: 11320- |
| | | 11321 |
| REPIN1 | Replication initiator 1 | SEQ ID NOS: 11322- |
| | | 11335 |
| REPS2 | RALBP1 associated Eps domain containing | SEQ ID NOS: 11336- |
| | 2 | 11337 |
| RET | Ret proto-oncogene | SEQ ID NOS: 11338- |
| | | 11343 |
| RETN | Resistin | SEQ ID NOS: 11344- |
| | | 11346 |
| RETNLB | Resistin like beta | SEQ ID NO: 11347 |
| RETSAT | Retinol saturase (all-trans-retinol 13,14- | SEQ ID NOS: 11348- |
| | reductase) | 11352 |
| RFNG | RFNG O-fucosylpeptide 3-beta-N- | SEQ ID NOS: 11353- |
| | acetylglucosaminyltransferase | 11355 |
| RGCC | Regulator of cell cycle | SEQ ID NO: 11356 |
| RGL4 | Ral guanine nucleotide dissociation | SEQ ID NOS: 11357- |
| | stimulator-like 4 | 11363 |
| RGMA | Repulsive guidance molecule family | SEQ ID NOS: 11364- |
| | member a | 11373 |
| RGMB | Repulsive guidance molecule family | SEQ ID NOS: 11374- |
| | member b | 11375 |
| RHOQ | Ras homolog family member Q | SEQ ID NOS: 11376- |
| | | 11380 |
| RIC3 | RIC3 acetylcholine receptor chaperone | SEQ ID NOS: 11381- |
| | | 11388 |
| RIMS1 | Regulating synaptic membrane exocytosis 1 | SEQ ID NOS: 11393- |
| | | 11408 |
| RIPPLY1 | Ripply transcriptional repressor 1 | SEQ ID NOS: 11409- |
| | | 11410 |
| RLN1 | Relaxin 1 | SEQ ID NO: 11411 |
| RLN2 | Relaxin 2 | SEQ ID NOS: 11412- |
| | | 11413 |
| RLN3 | Relaxin 3 | SEQ ID NOS: 11414- |
| | | 11415 |
| RMDN1 | Regulator of microtubule dynamics 1 | SEQ ID NOS: 11416- |
| | | 11429 |
| RNASE1 | Ribonuclease, RNase A family, 1 | SEQ ID NOS: 11430- |
| | (pancreatic) | 11434 |
| RNASE10 | Ribonuclease, RNase A family, 10 (non- | SEQ ID NOS: 11435- |
| | active) | 11436 |
| RNASE11 | Ribonuclease, RNase A family, 11 (non- | SEQ ID NOS: 11437- |
| | active) | 11447 |
| RNASE12 | Ribonuclease, RNase A family, 12 (non- | SEQ ID NO: 11448 |
| | active) | |
| RNASE13 | Ribonuclease, RNase A family, 13 (non- | SEQ ID NO: 11449 |
| | active) | |
| RNASE2 | Ribonuclease, RNase A family, 2 (liver, | SEQ ID NO: 11450 |
| | eosinophil-derived neurotoxin) | |
| RNASE3 | Ribonuclease, RNase A family, 3 | SEQ ID NO: 11451 |
| RNASE4 | Ribonuclease, RNase A family, 4 | SEQ ID NOS: 11452- |
| | | 11454 |
| RNASE6 | Ribonuclease, RNase A family, k6 | SEQ ID NO: 11455 |
| RNASE7 | Ribonuclease, RNase A family, 7 | SEQ ID NOS: 11456- |
| | | 11457 |
| RNASE8 | Ribonuclease, RNase A family, 8 | SEQ ID NO: 11458 |
| RNASE9 | Ribonuclease, RNase A family, 9 (non- | SEQ ID NOS: 11459- |
| | active) | 11469 |
| RNASEH1 | Ribonuclease H1 | SEQ ID NOS: 11470- |
| | | 11472 |
| RNASET2 | Ribonuclease T2 | SEQ ID NOS: 11473- |
| | | 11480 |
| RNF146 | Ring finger protein 146 | SEQ ID NOS: 11481- |
| | | 11492 |
| RNF148 | Ring finger protein 148 | SEQ ID NOS: 11493- |
| | | 11494 |
| RNF150 | Ring finger protein 150 | SEQ ID NOS: 11495- |
| | | 11499 |
| RNF167 | Ring finger protein 167 | SEQ ID NOS: 11500- |
| | | 11510 |
| RNF220 | Ring finger protein 220 | SEQ ID NOS: 11511- |
| | | 11517 |
| RNF34 | Ring finger protein 34, E3 ubiquitin protein | SEQ ID NOS: 11518- |
| | ligase | 11525 |
| RNLS | Renalase, FAD-dependent amine oxidase | SEQ ID NOS: 11526- |
| | | 11528 |
| RNPEP | Arginyl aminopeptidase (aminopeptidase B) | SEQ ID NOS: 11529- |
| | | 11534 |
| ROR1 | Receptor tyrosine kinase-like orphan | SEQ ID NOS: 11535- |
| | receptor 1 | 11537 |
| RP11- | | SEQ ID NO: 4158 |
| 1236K1.1 | | |
| RP11-14J7.7 | | SEQ ID NOS: 674-675 |
| RP11- | | SEQ ID NOS: 85-87 |
| 196G11.1 | | |
| RP11- | | SEQ ID NO: 683 |
| 350O14.18 | | |
| RP11- | | SEQ ID NO: 8194 |
| 520P18.5 | | |
| RP11- | | SEQ ID NO: 89 |
| 812E19.9 | | |
| RP11- | | SEQ ID NO: 676 |
| 903H12.5 | | |
| RP11- | | SEQ ID NOS: 78-80 |
| 977G19.10 | | |
| RP4-576H24.4 | | SEQ ID NOS: 670-672 |
| RP4-608O15.3 | Complement factor H-related protein 2 | SEQ ID NO: 1649 |
| RPL3 | Ribosomal protein L3 | SEQ ID NOS: 11538- |
| | | 11543 |
| RPLP2 | Ribosomal protein, large, P2 | SEQ ID NOS: 11544- |
| | | 11546 |
| RPN2 | Ribophorin II | SEQ ID NOS: 11547- |
| | | 11553 |
| RPS27L | Ribosomal protein S27-like | SEQ ID NOS: 11554- |
| | | 11559 |
| RQCD1 | RCD1 required for cell differentiation1 | SEQ ID NOS: 3100- |
| | homolog (S. pombe) | 3106 |
| RS1 | Retinoschisin 1 | SEQ ID NO: 11560 |
| RSF1 | Remodeling and spacing factor 1 | SEQ ID NOS: 11561- |
| | | 11567 |
| RSPO1 | R-spondin 1 | SEQ ID NOS: 11568- |
| | | 11571 |
| RSPO2 | R-spondin 2 | SEQ ID NOS: 11572- |
| | | 11579 |
| RSPO3 | R-spondin 3 | SEQ ID NOS: 11580- |
| | | 11581 |
| RSPO4 | R-spondin 4 | SEQ ID NOS: 11582- |
| | | 11583 |
| RSPRY1 | Ring finger and SPRY domain containing 1 | SEQ ID NOS: 11584- |
| | | 11590 |
| RTBDN | Retbindin | SEQ ID NOS: 11591- |
| | | 11603 |
| RTN4RL1 | Reticulon 4 receptor-like 1 | SEQ ID NO: 11604 |
| RTN4RL2 | Reticulon 4 receptor-like 2 | SEQ ID NOS: 11605- |
| | | 11607 |
| SAA1 | Serum amyloid A1 | SEQ ID NOS: 11608- |
| | | 11610 |
| SAA2 | Serum amyloid A2 | SEQ ID NOS: 11611- |
| | | 11616 |
| SAA4 | Serum amyloid A4, constitutive | SEQ ID NO: 11617 |
| SAP30 | Sin3A-associated protein, 30 kDa | SEQ ID NO: 11618 |
| SAR1A | Secretion associated, Ras related GTPase | SEQ ID NOS: 11619- |
| | 1A | 11625 |
| SARAF | Store-operated calcium entry-associated | SEQ ID NOS: 11626- |
| | regulatory factor | 11636 |
| SARM1 | Sterile alpha and TIR motif containing 1 | SEQ ID NOS: 11637- |
| | | 11640 |
| SATB1 | SATB homeobox 1 | SEQ ID NOS: 11641- |
| | | 11653 |
| SAXO2 | Stabilizer of axonemal microtubules 2 | SEQ ID NOS: 11654- |
| | | 11658 |
| SBSN | Suprabasin | SEQ ID NOS: 11659- |
| | | 11661 |
| SBSPON | Somatomedin B and thrombospondin, type | SEQ ID NO: 11662 |
| | 1 domain containing | |
| SCARF1 | Scavenger receptor class F, member 1 | SEQ ID NOS: 11663- |
| | | 11667 |
| SCG2 | Secretogranin II | SEQ ID NOS: 11668- |
| | | 11670 |
| SCG3 | Secretogranin III | SEQ ID NOS: 11671- |
| | | 11673 |
| SCG5 | Secretogranin V | SEQ ID NOS: 11674- |
| | | 11678 |
| SCGB1A1 | Secretoglobin, family 1A, member 1 | SEQ ID NOS: 11679- |
| | (uteroglobin) | 11680 |
| SCGB1C1 | Secretoglobin, family 1C, member 1 | SEQ ID NO: 11681 |
| SCGB1C2 | Secretoglobin, family 1C, member 2 | SEQ ID NO: 11682 |
| SCGB1D1 | Secretoglobin, family 1D, member 1 | SEQ ID NO: 11683 |
| SCGB1D2 | Secretoglobin, family 1D, member 2 | SEQ ID NO: 11684 |
| SCGB1D4 | Secretoglobin, family 1D, member 4 | SEQ ID NO: 11685 |
| SCGB2A1 | Secretoglobin, family 2A, member 1 | SEQ ID NO: 11686 |
| SCGB2A2 | Secretoglobin, family 2A, member 2 | SEQ ID NOS: 11687- |
| | | 11688 |
| SCGB2B2 | Secretoglobin, family 2B, member 2 | SEQ ID NOS: 11689- |
| | | 11690 |
| SCGB3A1 | Secretoglobin, family 3A, member 1 | SEQ ID NO: 11691 |
| SCGB3A2 | Secretoglobin, family 3A, member 2 | SEQ ID NOS: 11692- |
| | | 11693 |
| SCN1B | Sodium channel, voltage gated, type I beta | SEQ ID NOS: 11694- |
| | subunit | 11699 |
| SCN3B | Sodium channel, voltage gated, type III beta | SEQ ID NOS: 11700- |
| | subunit | 11704 |
| SCPEP1 | Serine carboxypeptidase 1 | SEQ ID NOS: 11705- |
| | | 11712 |
| SCRG1 | Stimulator of chondrogenesis 1 | SEQ ID NOS: 11713- |
| | | 11714 |
| SCT | Secretin | SEQ ID NO: 11715 |
| SCUBE1 | Signal peptide, CUB domain, EGF-like 1 | SEQ ID NOS: 11716- |
| | | 11719 |
| SCUBE2 | Signal peptide, CUB domain, EGF-like 2 | SEQ ID NOS: 11720- |
| | | 11726 |
| SCUBE3 | Signal peptide, CUB domain, EGF-like 3 | SEQ ID NO: 11727 |
| SDC1 | Syndecan 1 | SEQ ID NOS: 11728- |
| | | 11732 |
| SDF2 | Stromal cell-derived factor 2 | SEQ ID NOS: 11733- |
| | | 11735 |
| SDF2L1 | Stromal cell-derived factor 2-like 1 | SEQ ID NO: 11736 |
| SDF4 | Stromal cell derived factor 4 | SEQ ID NOS: 11737- |
| | | 11740 |
| SDHAF2 | Succinate dehydrogenase complex assembly | SEQ ID NOS: 11741- |
| | factor 2 | 11748 |
| SDHAF4 | Succinate dehydrogenase complex assembly | SEQ ID NO: 11749 |
| | factor 4 | |
| SDHB | Succinate dehydrogenase complex, subunit | SEQ ID NOS: 11750- |
| | B, iron sulfur (Ip) | 11752 |
| SDHD | Succinate dehydrogenase complex, subunit | SEQ ID NOS: 11753- |
| | D, integral membrane protein | 11762 |
| SEC14L3 | SEC14-like lipid binding 3 | SEQ ID NOS: 11763- |
| | | 11769 |
| SEC16A | SEC16 homolog A, endoplasmic reticulum | SEQ ID NOS: 11770- |
| | export factor | 11776 |
| SEC16B | SEC16 homolog B, endoplasmic reticulum | SEQ ID NOS: 11777- |
| | export factor | 11780 |
| SEC22C | SEC22 homolog C, vesicle trafficking | SEQ ID NOS: 11781- |
| | protein | 11793 |
| SEC31A | SEC31 homolog A, COPII coat complex | SEQ ID NOS: 11794- |
| | component | 11823 |
| SECISBP2 | SECIS binding protein 2 | SEQ ID NOS: 11824- |
| | | 11828 |
| SECTM1 | Secreted and transmembrane 1 | SEQ ID NOS: 11829- |
| | | 11836 |
| SEL1L | Sel-1 suppressor of lin-12-like (C. elegans) | SEQ ID NOS: 11837- |
| | | 11839 |
| SELM | Selenoprotein M | SEQ ID NOS: 11847- |
| | | 11849 |
| SELO | Selenoprotein O | SEQ ID NOS: 11854- |
| | | 11855 |
| SEMA3A | Sema domain, immunoglobulin domain | SEQ ID NOS: 11862- |
| | (Ig), short basic domain, secreted, | 11866 |
| | (semaphorin) 3A | |
| SEMA3B | Sema domain, immunoglobulin domain | SEQ ID NOS: 11867- |
| | (Ig), short basic domain, secreted, | 11873 |
| | (semaphorin) 3B | |
| SEMA3C | Sema domain, immunoglobulin domain | SEQ ID NOS: 11874- |
| | (Ig), short basic domain, secreted, | 11878 |
| | (semaphorin) 3C | |
| SEMA3E | Sema domain, immunoglobulin domain | SEQ ID NOS: 11879- |
| | (Ig), short basic domain, secreted, | 11883 |
| | (semaphorin) 3E | |
| SEMA3F | Sema domain, immunoglobulin domain | SEQ ID NOS: 11884- |
| | (Ig), short basic domain, secreted, | 11890 |
| | (semaphorin) 3F | |
| SEMA3G | Sema domain, immunoglobulin domain | SEQ ID NOS: 11891- |
| | (Ig), short basic domain, secreted, | 11893 |
| | (semaphorin) 3G | |
| SEMA4A | Sema domain, immunoglobulin domain | SEQ ID NOS: 11894- |
| | (Ig), transmembrane domain (TM) and short | 11902 |
| | cytoplasmic domain, (semaphorin) 4A | |
| SEMA4B | Sema domain, immunoglobulin domain | SEQ ID NOS: 11903- |
| | (Ig), transmembrane domain (TM) and short | 11913 |
| | cytoplasmic domain, (semaphorin) 4B | |
| SEMA4C | Sema domain, immunoglobulin domain | SEQ ID NOS: 11914- |
| | (Ig), transmembrane domain (TM) and short | 11916 |
| | cytoplasmic domain, (semaphorin) 4C | |
| SEMA4D | Sema domain, immunoglobulin domain | SEQ ID NOS: 11917- |
| | (Ig), transmembrane domain (TM) and short | 11930 |
| | cytoplasmic domain, (semaphorin) 4D | |
| SEMA4F | Sema domain, immunoglobulin domain | SEQ ID NOS: 11931- |
| | (Ig), transmembrane domain (TM) and short | 11939 |
| | cytoplasmic domain, (semaphorin) 4F | |
| SEMA4G | Sema domain, immunoglobulin domain | SEQ ID NOS: 11940- |
| | (Ig), transmembrane domain (TM) and short | 11947 |
| | cytoplasmic domain, (semaphorin) 4G | |
| SEMA5A | Sema domain, seven thrombospondin | SEQ ID NOS: 11948- |
| | repeats (type 1 and type 1-like), | 11949 |
| | transmembrane domain (TM) and short | |
| | cytoplasmic domain, (semaphorin) 5A | |
| SEMA6A | Sema domain, transmembrane domain | SEQ ID NOS: 11950- |
| | (TM), and cytoplasmic domain, | 11957 |
| | (semaphorin) 6A | |
| SEMA6C | Sema domain, transmembrane domain | SEQ ID NOS: 11958- |
| | (TM), and cytoplasmic domain, | 11963 |
| | (semaphorin) 6C | |
| SEMA6D | Sema domain, transmembrane domain | SEQ ID NOS: 11964- |
| | (TM), and cytoplasmic domain, | 11977 |
| | (semaphorin) 6D | |
| SEMG1 | Semenogelin I | SEQ ID NO: 11978 |
| SEMG2 | Semenogelin II | SEQ ID NO: 11979 |
| SEPN1 | Selenoprotein N, 1 | SEQ ID NOS: 11850- |
| | | 11853 |
| SEPP1 | Selenoprotein P, plasma, 1 | SEQ ID NOS: 11856- |
| | | 11861 |
| SEPT15 | 15 kDa selenoprotein | SEQ ID NOS: 11840- |
| | | 11846 |
| SEPT9 | Septin 9 | SEQ ID NOS: 11980- |
| | | 12016 |
| SERPINA1 | Serpin peptidase inhibitor, clade A (alpha-1 | SEQ ID NOS: 12017- |
| | antiproteinase, antitrypsin), member 1 | 12033 |
| SERPINA10 | Serpin peptidase inhibitor, clade A (alpha-1 | SEQ ID NOS: 12034- |
| | anti proteinase, antitrypsin), member 10 | 12037 |
| SERPINA11 | Serpin peptidase inhibitor, clade A (alpha-1 | SEQ ID NO: 12038 |
| | antiproteinase, antitrypsin), member 11 | |
| SERPINA12 | Serpin peptidase inhibitor, clade A (alpha-1 | SEQ ID NOS: 12039- |
| | anti proteinase, antitrypsin), member 12 | 12040 |
| SERPINA3 | Serpin peptidase inhibitor, clade A (alpha-1 | SEQ ID NOS: 673- |
| | antiproteinase, antitrypsin), member 3 | 12047 |
| SERPINA4 | Serpin peptidase inhibitor, clade A (alpha-1 | SEQ ID NOS: 12048- |
| | antiproteinase, antitrypsin), member 4 | 12050 |
| SERPINA5 | Serpin peptidase inhibitor, clade A (alpha-1 | SEQ ID NOS: 12051- |
| | antiproteinase, antitrypsin), member 5 | 12062 |
| SERPINA6 | Serpin peptidase inhibitor, clade A (alpha-1 | SEQ ID NOS: 12063- |
| | antiproteinase, antitrypsin), member 6 | 12065 |
| SERPINA7 | Serpin peptidase inhibitor, clade A (alpha-1 | SEQ ID NOS: 12066- |
| | antiproteinase, antitrypsin), member 7 | 12067 |
| SERPINA9 | Serpin peptidase inhibitor, clade A (alpha-1 | SEQ ID NOS: 12068- |
| | antiproteinase, antitrypsin), member 9 | 12074 |
| SERPINB2 | Serpin peptidase inhibitor, clade B | SEQ ID NOS: 12075- |
| | (ovalbumin), member 2 | 12079 |
| SERPINC1 | Serpin peptidase inhibitor, clade C | SEQ ID NOS: 12080- |
| | (antithrombin), member 1 | 12081 |
| SERPIND1 | Serpin peptidase inhibitor, clade D (heparin | SEQ ID NOS: 12082- |
| | cofactor), member 1 | 12083 |
| SERPINE1 | Serpin peptidase inhibitor, clade E (nexin. | SEQ ID NO: 12084 |
| | plasminogen activator inhibitor type 1), | |
| | member 1 | |
| SERPINE2 | Serpin peptidase inhibitor, clade E (nexin, | SEQ ID NOS: 12085- |
| | plasminogen activator inhibitor type 1), | 12091 |
| | member 2 | |
| SERPINE3 | Serpin peptidase inhibitor, clade E (nexin, | SEQ ID NOS: 12092- |
| | plasminogen activator inhibitor type 1), | 12095 |
| | member 3 | |
| SERPINF1 | Serpin peptidase inhibitor, clade F (alpha-2 | SEQ ID NOS: 12096- |
| | antiplasmin, pigment epithelium derived | 12104 |
| | factor), member 1 | |
| SERPINF2 | Serpin peptidase inhibitor, clade F (alpha-2 | SEQ ID NOS: 12105- |
| | antiplasmin, pigment epithelium derived | 12109 |
| | factor), member 2 | |
| SERPING1 | Serpin peptidase inhibitor, clade G (C1 | SEQ ID NOS: 12110- |
| | inhibitor), member 1 | 12120 |
| SERPINH1 | Serpin peptidase inhibitor, clade H (heat | SEQ ID NOS: 12121- |
| | shock protein 47), member 1, (collagen | 12135 |
| | binding protein 1) | |
| SERPINI1 | Serpin peptidase inhibitor, clade I | SEQ ID NOS: 12136- |
| | (neuroserpin), member 1 | 12140 |
| SERPINI2 | Serpin peptidase inhibitor, clade I (pancpin), | SEQ ID NOS: 12141- |
| | member 2 | 12147 |
| SETD8 | SET domain containing (lysine | SEQ ID NOS: 7589- |
| | methyltransferase) 8 | 7592 |
| SEZ6L2 | Seizure related 6 homolog (mouse)-like 2 | SEQ ID NOS: 12148- |
| | | 12154 |
| SFRP1 | Secreted frizzled-related protein 1 | SEQ ID NOS: 12155- |
| | | 12156 |
| SFRP2 | Secreted frizzled-related protein 2 | SEQ ID NO: 12157 |
| SFRP4 | Secreted frizzled-related protein 4 | SEQ ID NOS: 12158- |
| | | 12159 |
| SFRP5 | Secreted frizzled-related protein 5 | SEQ ID NO: 12160 |
| SFTA2 | Surfactant associated 2 | SEQ ID NOS: 12161- |
| | | 12162 |
| SFTPA1 | Surfactant protein A1 | SEQ ID NOS: 12163- |
| | | 12167 |
| SFTPA2 | Surfactant protein A2 | SEQ ID NOS: 12168- |
| | | 12172 |
| SFTPB | Surfactant protein B | SEQ ID NOS: 12173- |
| | | 12177 |
| SFTPD | Surfactant protein D | SEQ ID NOS: 12178- |
| | | 12179 |
| SFXN5 | Sideroflexin 5 | SEQ ID NOS: 12180- |
| | | 12184 |
| SGCA | Sarcoglycan, alpha (50 kDa dystrophin- | SEQ ID NOS: 12185- |
| | associated glycoprotein) | 12192 |
| SGSH | N-sulfoglucosamine sulfohydrolase | SEQ ID NOS: 12193- |
| | | 12201 |
| SH3RF3 | SH3 domain containing ring finger 3 | SEQ ID NO: 12202 |
| SHBG | Sex hormone-binding globulin | SEQ ID NOS: 12203- |
| | | 12221 |
| SHE | Src homology 2 domain containing E | SEQ ID NOS: 12222- |
| | | 12224 |
| SHH | Sonic hedgehog | SEQ ID NOS: 12225- |
| | | 12228 |
| SHKBP1 | SH3KBP1 binding protein 1 | SEQ ID NOS: 12229- |
| | | 12244 |
| SIAE | Sialic acid acetylesterase | SEQ ID NOS: 12245- |
| | | 12247 |
| SIDT2 | SID1 transmembrane family, member 2 | SEQ ID NOS: 12248- |
| | | 12257 |
| SIGLEC10 | Sialic acid binding Ig-like lectin 10 | SEQ ID NOS: 12258- |
| | | 12266 |
| SIGLEC6 | Sialic acid binding Ig-like lectin 6 | SEQ ID NOS: 12267- |
| | | 12272 |
| SIGLEC7 | Sialic acid binding Ig-like lectin 7 | SEQ ID NOS: 12273- |
| | | 12277 |
| SIGLECL1 | SIGLEC family like 1 | SEQ ID NOS: 12278- |
| | | 12283 |
| SIGMAR1 | Sigma non-opioid intracellular receptor 1 | SEQ ID NOS: 12284- |
| | | 12287 |
| SIL1 | SIL1 nucleotide exchange factor | SEQ ID NOS: 12288- |
| | | 12296 |
| SIRPB1 | Signal-regulatory protein beta 1 | SEQ ID NOS: 12297- |
| | | 12309 |
| SIRPD | Signal-regulatory protein delta | SEQ ID NOS: 12310- |
| | | 12312 |
| SLAMF1 | Signaling lymphocytic activation molecule | SEQ ID NOS: 12313- |
| | family member 1 | 12315 |
| SLAMF7 | SLAM family member 7 | SEQ ID NOS: 12316- |
| | | 12324 |
| SLC10A3 | Solute carrier family 10, member 3 | SEQ ID NOS: 12325- |
| | | 12329 |
| SLC15A3 | Solute carrier family 15 (oligopeptide | SEQ ID NOS: 12330- |
| | transporter), member 3 | 12335 |
| SLC25A14 | Solute carrier family 25 (mitochondrial | SEQ ID NOS: 12336- |
| | carrier, brain), member 14 | 12342 |
| SLC25A25 | Solute carrier family 25 (mitochondrial | SEQ ID NOS: 12343- |
| | carrier; phosphate carrier), member 25 | 12349 |
| SLC2A5 | Solute carrier family 2 (facilitated | SEQ ID NOS: 12350- |
| | glucose/fructose transporter), member 5 | 12358 |
| SLC35E3 | Solute carrier family 35, member E3 | SEQ ID NOS: 12359- |
| | | 12360 |
| SLC39A10 | Solute carrier family 39 (zinc transporter), | SEQ ID NOS: 12361- |
| | member 10 | 12367 |
| SLC39A14 | Solute carrier family 39 (zinc transporter), | SEQ ID NOS: 12368- |
| | member 14 | 12378 |
| SLC39A4 | Solute carrier family 39 (zinc transporter), | SEQ ID NOS: 12379- |
| | member 4 | 12381 |
| SLC39A5 | Solute carrier family 39 (zinc transporter), | SEQ ID NOS: 12382- |
| | member 5 | 12388 |
| SLC3A1 | Solute carrier family 3 (amino acid | SEQ ID NOS: 12389- |
| | transporter heavy chain), member 1 | 12398 |
| SLC51A | Solute carrier family 51, alpha subunit | SEQ ID NOS: 12399- |
| | | 12403 |
| SLC52A2 | Solute carrier family 52 (riboflavin | SEQ ID NOS: 12404- |
| | transporter), member 2 | 12414 |
| SLC5A6 | Solute carrier family 5 | SEQ ID NOS: 12415- |
| | (sodium/multivitamin and iodide | 12425 |
| | cotransporter), member 6 | |
| SLC6A9 | Solute carrier family 6 (neurotransmitter | SEQ ID NOS: 12426- |
| | transporter, glycine), member 9 | 12433 |
| SLC8A1 | Solute carrier family 8 (sodium/calcium | SEQ ID NOS: 12434- |
| | exchanger), member 1 | 12445 |
| SLC8B1 | Solute carrier family 8 | SEQ ID NOS: 12446- |
| | (sodium/lithium/calcium exchanger), | 12456 |
| | member B1 | |
| SLC9A6 | Solute carrier family 9, subfamily A | SEQ ID NOS: 12457- |
| | (NHE6, cation proton antiporter 6), member | 12468 |
| | 6 | |
| SLCO1A2 | Solute carrier organic anion transporter | SEQ ID NOS: 12469- |
| | family, member 1A2 | 12481 |
| SLIT1 | Slit guidance ligand 1 | SEQ ID NOS: 12482- |
| | | 12485 |
| SLIT2 | Slit guidance ligand 2 | SEQ ID NOS: 12486- |
| | | 12494 |
| SLIT3 | Slit guidance ligand 3 | SEQ ID NOS: 12495- |
| | | 12497 |
| SLITRK3 | SLIT and NTRK-like family, member 3 | SEQ ID NOS: 12498- |
| | | 12500 |
| SLPI | Secretory leukocyte peptidase inhibitor | SEQ ID NO: 12501 |
| SLTM | SAFB-like, transcription modulator | SEQ ID NOS: 12502- |
| | | 12515 |
| SLURP1 | Secreted LY6/PLAUR domain containing 1 | SEQ ID NO: 12516 |
| SMARCA2 | SWI/SNF related, matrix associated, actin | SEQ ID NOS: 12517- |
| | dependent regulator of chromatin, subfamily | 12562 |
| | a, member 2 | |
| SMG6 | SMG6 nonsense mediated mRNA decay | SEQ ID NOS: 12563- |
| | factor | 12574 |
| SMIM7 | Small integral membrane protein 7 | SEQ ID NOS: 12575- |
| | | 12591 |
| SMOC1 | SPARC related modular calcium binding 1 | SEQ ID NOS: 12592- |
| | | 12593 |
| SMOC2 | SPARC related modular calcium binding 2 | SEQ ID NOS: 12594- |
| | | 12598 |
| SMPDL3A | Sphingomyelin phosphodiesterase, acid-like | SEQ ID NOS: 12599- |
| | 3A | 12600 |
| SMPDL3B | Sphingomyelin phosphodiesterase, acid-like | SEQ ID NOS: 12601- |
| | 3B | 12605 |
| SMR3A | Submaxillary gland androgen regulated | SEQ ID NO: 12606 |
| | protein 3A | |
| SMR3B | Submaxillary gland androgen regulated | SEQ ID NOS: 12607- |
| | protein 3B | 12609 |
| SNED1 | Sushi, nidogen and EGF-like domains 1 | SEQ ID NOS: 12610- |
| | | 12616 |
| SNTB1 | Syntrophin, beta 1 (dystrophin-associated | SEQ ID NOS: 12617- |
| | protein A1, 59 kDa, basic component 1) | 12619 |
| SNTB2 | Syntrophin, beta 2 (dystrophin-associated | SEQ ID NOS: 12620- |
| | protein A1, 59 kDa, basic component 2) | 12624 |
| SNX14 | Sorting nexin 14 | SEQ ID NOS: 12625- |
| | | 12636 |
| SOD3 | Superoxide dismutase 3, extracellular | SEQ ID NOS: 12637- |
| | | 12638 |
| SOST | Sclerostin | SEQ ID NO: 12639 |
| SOSTDC1 | Sclerostin domain containing 1 | SEQ ID NOS: 12640- |
| | | 12641 |
| SOWAHA | Sosondowah ankyrin repeat domain family | SEQ ID NO: 12642 |
| | member A | |
| SPACA3 | Sperm acrosome associated 3 | SEQ ID NOS: 12643- |
| | | 12645 |
| SPACA4 | Sperm acrosome associated 4 | SEQ ID NO: 12646 |
| SPACA5 | Sperm acrosome associated 5 | SEQ ID NOS: 12647- |
| | | 12648 |
| SPACA5B | Sperm acrosome associated 5B | SEQ ID NO: 12649 |
| SPACA7 | Sperm acrosome associated 7 | SEQ ID NOS: 12650- |
| | | 12653 |
| SPAG11A | Sperm associated antigen 11A | SEQ ID NOS: 12654- |
| | | 12662 |
| SPAG11B | Sperm associated antigen 11B | SEQ ID NOS: 12663- |
| | | 12671 |
| SPARC | Secreted protein, acidic, cysteine-rich | SEQ ID NOS: 12672- |
| | (osteonectin) | 12676 |
| SPARCL1 | SPARC-like 1 (hevin) | SEQ ID NOS: 12677- |
| | | 12686 |
| SPATA20 | Spermatogenesis associated 20 | SEQ ID NOS: 12687- |
| | | 12700 |
| SPESP1 | Sperm equatorial segment protein 1 | SEQ ID NO: 12701 |
| SPINK1 | Serine peptidase inhibitor, Kazal type 1 | SEQ ID NOS: 12702- |
| | | 12703 |
| SPINK13 | Serine peptidase inhibitor, Kazal type 13 | SEQ ID NOS: 12704- |
| | (putative) | 12706 |
| SPINK14 | Serine peptidase inhibitor, Kazal type 14 | SEQ ID NOS: 12707- |
| | (putative) | 12708 |
| SPINK2 | Serine peptidase inhibitor, Kazal type 2 | SEQ ID NOS: 12709- |
| | (acrosin-trypsin inhibitor) | 12714 |
| SPINK4 | Serine peptidase inhibitor, Kazal type 4 | SEQ ID NOS: 12715- |
| | | 12716 |
| SPINK5 | Serine peptidase inhibitor, Kazal type 5 | SEQ ID NOS: 12717- |
| | | 12722 |
| SPINK6 | Serine peptidase inhibitor, Kazal type 6 | SEQ ID NOS: 12723- |
| | | 12725 |
| SPINK7 | Serine peptidase inhibitor, Kazal type 7 | SEQ ID NOS: 12726- |
| | (putative) | 12727 |
| SPINK8 | Serine peptidase inhibitor, Kazal type 8 | SEQ ID NO: 12728 |
| | (putative) | |
| SPINK9 | Serine peptidase inhibitor, Kazal type 9 | SEQ ID NOS: 12729- |
| | | 12730 |
| SPINT1 | Serine peptidase inhibitor, Kunitz type 1 | SEQ ID NOS: 12731- |
| | | 12738 |
| SPINT2 | Serine peptidase inhibitor, Kunitz type, 2 | SEQ ID NOS: 12739- |
| | | 12746 |
| SPINT3 | Serine peptidase inhibitor, Kunitz type, 3 | SEQ ID NO: 12747 |
| SPINT4 | Serine peptidase inhibitor, Kunitz type 4 | SEQ ID NO: 12748 |
| SPOCK1 | Sparc/osteonectin, cwcv and kazal-like | SEQ ID NOS: 12749- |
| | domains proteoglycan (testican) 1 | 12752 |
| SPOCK2 | Sparc/osteonectin, cwcv and kazal-like | SEQ ID NOS: 12753- |
| | domains proteoglycan (testican) 2 | 12756 |
| SPOCK3 | Sparc/osteonectin, cwcv and kazal-like | SEQ ID NOS: 12757- |
| | domains proteoglycan (testican) 3 | 12782 |
| SPON1 | Spondin 1, extracellular matrix protein | SEQ ID NO: 12783 |
| SPON2 | Spondin 2, extracellular matrix protein | SEQ ID NOS: 12784- |
| | | 12793 |
| SPP1 | Secreted phosphoprotein 1 | SEQ ID NOS: 12794- |
| | | 12798 |
| SPP2 | Secreted phosphoprotein 2, 24 kDa | SEQ ID NOS: 12799- |
| | | 12801 |
| SPRN | Shadow of prion protein homolog | SEQ ID NO: 12802 |
| | (zebrafish) | |
| SPRYD3 | SPRY domain containing 3 | SEQ ID NOS: 12803- |
| | | 12806 |
| SPRYD4 | SPRY domain containing 4 | SEQ ID NO: 12807 |
| SPTY2D1-AS1 | SPTY2D1 antisense RNA 1 | SEQ ID NOS: 12808- |
| | | 12813 |
| SPX | Spexin hormone | SEQ ID NOS: 12814- |
| | | 12815 |
| SRGN | Serglycin | SEQ ID NO: 12816 |
| SRL | Sarealumenin | SEQ ID NOS: 12817- |
| | | 12819 |
| SRP14 | Signal recognition particle 14 kDa | SEQ ID NOS: 12820- |
| | (homologous Alu RNA binding protein) | 12823 |
| SRPX | Sushi-repeat containing protein, X-linked | SEQ ID NOS: 12824- |
| | | 12827 |
| SRPX2 | Sushi-repeat containing protein, X-linked 2 | SEQ ID NOS: 12828- |
| | | 12831 |
| SSC4D | Scavenger receptor cysteine rich family, 4 | SEQ ID NO: 12832 |
| | domains | |
| SSC5D | Scavenger receptor cysteine rich family, 5 | SEQ ID NOS: 12833- |
| | domains | 12836 |
| SSPO | SCO-spondin | SEQ ID NO: 12837 |
| SSR2 | Signal sequence receptor, beta (translocon- | SEQ ID NOS: 12838- |
| | associated protein beta) | 12847 |
| SST | Somatostatin | SEQ ID NO: 12848 |
| ST3GAL1 | ST3 beta-galactoside alpha-2,3- | SEQ ID NOS: 12849- |
| | sialyltransferase 1 | 12856 |
| ST3GAL4 | ST3 beta-galactoside alpha-2,3- | SEQ ID NOS: 12857- |
| | sialyltransferase 4 | 12872 |
| ST6GAL1 | ST6 beta-galactosamide alpha-2,6- | SEQ ID NOS: 12873- |
| | sialyltranferase 1 | 12888 |
| ST6GALNAC2 | ST6 (alpha-N-acetyl-neuraminyl-2,3-beta- | SEQ ID NOS: 12889- |
| | galactosyl-1,3)-N-acetylgalactosaminide | 12893 |
| | alpha-2,6-sialyltransferase 2 | |
| ST6GALNAC5 | ST6 (alpha-N-acetyl-neuraminyl-2,3-beta- | SEQ ID NOS: 12894- |
| | galactosyl-1,3)-N-acetylgalactosaminide | 12895 |
| | alpha-2,6-sialyltransferase 5 | |
| ST6GALNAC6 | ST6 (alpha-N-acetyl-neuraminyl-2,3-beta- | SEQ ID NOS: 12896- |
| | galactosyl-1,3)-N-acetylgalactosaminide | 12903 |
| | alpha-2,6-sialyltransferase 6 | |
| ST8SIA2 | ST8 alpha-N-acetyl-neuraminide alpha-2,8- | SEQ ID NOS: 12904- |
| | sialyltransferase 2 | 12906 |
| ST8SIA4 | ST8 alpha-N-acetyl-neuraminide alpha-2,8- | SEQ ID NOS: 12907- |
| | sialyltransferase 4 | 12909 |
| ST8SIA6 | ST8 alpha-N-acetyl-neuraminide alpha-2,8- | SEQ ID NOS: 12910- |
| | sialyltransferase 6 | 12911 |
| STARD7 | StAR-related lipid transfer (START) | SEQ ID NOS: 12912- |
| | domain containing 7 | 12913 |
| STATH | Statherin | SEQ ID NOS: 12914- |
| | | 12916 |
| STC1 | Stanniocalcin 1 | SEQ ID NOS: 12917- |
| | | 12918 |
| STC2 | Stanniocalcin 2 | SEQ ID NOS: 12919- |
| | | 12921 |
| STMND1 | Stathmin domain containing 1 | SEQ ID NOS: 12922- |
| | | 12923 |
| STOML2 | Stomatin (EPB72)-like 2 | SEQ ID NOS: 12926- |
| | | 12929 |
| STOX1 | Storkhead box 1 | SEQ ID NOS: 12930- |
| | | 12934 |
| STRC | Stereocilin | SEQ ID NOS: 12935- |
| | | 12940 |
| SUCLG1 | Succinate-CoA ligase, alpha subunit | SEQ ID NOS: 12941- |
| | | 12942 |
| SUDS3 | SDS3 homolog, SIN3A corepressor | SEQ ID NO: 12943 |
| | complex component | |
| SULF1 | Sulfatase 1 | SEQ ID NOS: 12944- |
| | | 12954 |
| SULF2 | Sulfatase 2 | SEQ ID NOS: 12955- |
| | | 12959 |
| SUMF1 | Sulfatase modifying factor 1 | SEQ ID NOS: 12960- |
| | | 12964 |
| SUMF2 | Sulfatase modifying factor 2 | SEQ ID NOS: 12965- |
| | | 12978 |
| SUSD1 | Sushi domain containing 1 | SEQ ID NOS: 12979- |
| | | 12984 |
| SUSD5 | Sushi domain containing 5 | SEQ ID NOS: 12985- |
| | | 12986 |
| SVEP1 | Sushi, von Willebrand factor type A, EGF | SEQ ID NOS: 12987- |
| | and pentraxin domain containing 1 | 12989 |
| SWSAP1 | SWIM-type zinc finger 7 associated protein | SEQ ID NO: 12990 |
| | 1 | |
| SYAP1 | Synapse associated protein 1 | SEQ ID NO: 12991 |
| SYCN | Syncollin | SEQ ID NO: 12992 |
| TAC1 | Tachykinin, precursor 1 | SEQ ID NOS: 12993- |
| | | 12995 |
| TAC3 | Tachykinin 3 | SEQ ID NOS: 12996- |
| | | 13005 |
| TAC4 | Tachykinin 4 (hemokinin) | SEQ ID NOS: 13006- |
| | | 13011 |
| TAGLN2 | Transgelin 2 | SEQ ID NOS: 13012- |
| | | 13015 |
| TAPBP | TAP binding protein (tapasin) | SEQ ID NOS: 13016- |
| | | 13021 |
| TAPBPL | TAP binding protein-like | SEQ ID NOS: 13022- |
| | | 13023 |
| TBL2 | Transducin (beta)-like 2 | SEQ ID NOS: 13024- |
| | | 13036 |
| TBX10 | T-box 10 | SEQ ID NO: 13037 |
| TCF12 | Transcription factor 12 | SEQ ID NOS: 13038- |
| | | 13051 |
| TCN1 | Transcobalamin I (vitamin B12 binding | SEQ ID NO: 13052 |
| | protein, R binder family) | |
| TCN2 | Transcobalamin II | SEQ ID NOS: 13053- |
| | | 13056 |
| TCTN1 | Tectonic family member 1 | SEQ ID NOS: 13057- |
| | | 13075 |
| TCTN3 | Tectonic family member 3 | SEQ ID NOS: 13076- |
| | | 13080 |
| TDP2 | Tyrosyl-DNA phosphodiesterase 2 | SEQ ID NOS: 13081- |
| | | 13082 |
| TEK | TEK tyrosine kinase, endothelial | SEQ ID NOS: 13097- |
| | | 13101 |
| TEPP | Testis, prostate and placenta expressed | SEQ ID NOS: 13102- |
| | | 13103 |
| TEX101 | Testis expressed 101 | SEQ ID NOS: 13104- |
| | | 13105 |
| TEX264 | Testis expressed 264 | SEQ ID NOS: 13106- |
| | | 13117 |
| TF | Transferrin | SEQ ID NOS: 13121- |
| | | 13127 |
| TFAM | Transcription factor A, mitochondrial | SEQ ID NOS: 13128- |
| | | 13130 |
| TFF1 | Trefoil factor 1 | SEQ ID NO: 13131 |
| TFF2 | Trefoil factor 2 | SEQ ID NO: 13132 |
| TFF3 | Trefoil factor 3 (intestinal) | SEQ ID NOS: 13133- |
| | | 13135 |
| TFPI | Tissue factor pathway inhibitor (lipoprotein- | SEQ ID NOS: 13136- |
| | associated coagulation inhibitor) | 13145 |
| TFPI2 | Tissue factor pathway inhibitor 2 | SEQ ID NOS: 13146- |
| | | 13147 |
| TG | Thyroglobulin | SEQ ID NOS: 13148- |
| | | 13157 |
| TGFB1 | Transforming growth factor, beta 1 | SEQ ID NOS: 13158- |
| | | 13159 |
| TGFB2 | Transforming growth factor, beta 2 | SEQ ID NOS: 13160- |
| | | 13161 |
| TGFB3 | Transforming growth factor, beta 3 | SEQ ID NOS: 13162- |
| | | 13163 |
| TGFBI | Transforming growth factor, beta-induced, | SEQ ID NOS: 13164- |
| | 68 kDa | 13171 |
| TGFBR1 | Transforming growth factor, beta receptor 1 | SEQ ID NOS: 13172- |
| | | 13181 |
| TGFBR3 | Transforming growth factor, beta receptor | SEQ ID NOS: 13182- |
| | III | 13188 |
| THBS1 | Thrombospondin 1 | SEQ ID NOS: 13189- |
| | | 13190 |
| THBS2 | Thrombospondin 2 | SEQ ID NOS: 13191- |
| | | 13193 |
| THBS3 | Thrombospondin 3 | SEQ ID NOS: 13194- |
| | | 13198 |
| THBS4 | Thrombospondin 4 | SEQ ID NOS: 13199- |
| | | 13200 |
| THOC3 | THO complex 3 | SEQ ID NOS: 13201- |
| | | 13210 |
| THPO | Thrombopoietin | SEQ ID NOS: 13211- |
| | | 13216 |
| THSD4 | Thrombospondin, type I, domain containing | SEQ ID NOS: 13217- |
| | 4 | 13220 |
| THY1 | Thy-1 cell surface antigen | SEQ ID NOS: 13221- |
| | | 13226 |
| TIE1 | Tyrosine kinase with immunoglobulin-like | SEQ ID NOS: 13227- |
| | and EGF-like domains 1 | 13228 |
| TIMMDC1 | Translocase of inner mitochondrial | SEQ ID NOS: 13229- |
| | membrane domain containing 1 | 13236 |
| TIMP1 | TIMP metallopeptidase inhibitor 1 | SEQ ID NOS: 13237- |
| | | 13241 |
| TIMP2 | TIMP metallopeptidase inhibitor 2 | SEQ ID NOS: 13242- |
| | | 13246 |
| TIMP3 | TIMP metallopeptidase inhibitor 3 | SEQ ID NO: 13247 |
| TIMP4 | TIMP metallopeptidase inhibitor 4 | SEQ ID NO: 13248 |
| TINAGL1 | Tubulointerstitial nephritis antigen-like 1 | SEQ ID NOS: 13249- |
| | | 13251 |
| TINF2 | TERF1 (TRF1)-interacting nuclear factor 2 | SEQ ID NOS: 13252- |
| | | 13261 |
| TLL2 | Tolloid-like 2 | SEQ ID NO: 13262 |
| TLR1 | Toll-like receptor 1 | SEQ ID NOS: 13263- |
| | | 13268 |
| TLR3 | Toll-like receptor 3 | SEQ ID NOS: 13269- |
| | | 13271 |
| TM2D2 | TM2 domain containing 2 | SEQ ID NOS: 13272- |
| | | 13277 |
| TM2D3 | TM2 domain containing 3 | SEQ ID NOS: 13278- |
| | | 13285 |
| TM7SF3 | Transmembrane 7 superfamily member 3 | SEQ ID NOS: 13286- |
| | | 13300 |
| TM9SF1 | Transmembrane 9 superfamily member 1 | SEQ ID NOS: 13301- |
| | | 13311 |
| TMCO6 | Transmembrane and coiled-coil domains 6 | SEQ ID NOS: 13312- |
| | | 13319 |
| TMED1 | Transmembrane p24 trafficking protein 1 | SEQ ID NOS: 13320- |
| | | 13326 |
| TMED2 | Transmembrane p24 trafficking protein 2 | SEQ ID NOS: 13327- |
| | | 13329 |
| TMED3 | Transmembrane p24 trafficking protein 3 | SEQ ID NOS: 13330- |
| | | 13333 |
| TMED4 | Transmembrane p24 trafficking protein 4 | SEQ ID NOS: 13334- |
| | | 13336 |
| TMED5 | Transmembrane p24 trafficking protein 5 | SEQ ID NOS: 13337- |
| | | 13340 |
| TMED7 | Transmembrane p24 trafficking protein 7 | SEQ ID NOS: 13341- |
| | | 13342 |
| TMED7- | TMED7-TICAM2 readthrough | SEQ ID NOS: 13343- |
| TICAM2 | | 13344 |
| TMEM108 | Transmembrane protein 108 | SEQ ID NOS: 13345- |
| | | 13353 |
| TMEM116 | Transmembrane protein 116 | SEQ ID NOS: 13354- |
| | | 13365 |
| TMEM119 | Transmembrane protein 119 | SEQ ID NOS: 13366- |
| | | 13369 |
| TMEM155 | Transmembrane protein 155 | SEQ ID NOS: 13370- |
| | | 13373 |
| TMEM168 | Transmembrane protein 168 | SEQ ID NOS: 13374- |
| | | 13379 |
| TMEM178A | Transmembrane protein 178A | SEQ ID NOS: 13380- |
| | | 13381 |
| TMEM179 | Transmembrane protein 179 | SEQ ID NOS: 13382- |
| | | 13387 |
| TMEM196 | Transmembrane protein 196 | SEQ ID NOS: 13388- |
| | | 13392 |
| TMEM199 | Transmembrane protein 199 | SEQ ID NOS: 13393- |
| | | 13396 |
| TMEM205 | Transmembrane protein 205 | SEQ ID NOS: 13397- |
| | | 13410 |
| TMEM213 | Transmembrane protein 213 | SEQ ID NOS: 13411- |
| | | 13414 |
| TMEM25 | Transmembrane protein 25 | SEQ ID NOS: 13415- |
| | | 13431 |
| TMEM30C | Transmembrane protein 30C | SEQ ID NO: 13432 |
| TMEM38B | Transmembrane protein 38B | SEQ ID NOS: 13433- |
| | | 13437 |
| TMEM44 | Transmembrane protein 44 | SEQ ID NOS: 13438- |
| | | 13447 |
| TMEM52 | Transmembrane protein 52 | SEQ ID NOS: 13448- |
| | | 13452 |
| TMEM52B | Transmembrane protein 52B | SEQ ID NOS: 13453- |
| | | 13455 |
| TMEM59 | Transmembrane protein 59 | SEQ ID NOS: 13456- |
| | | 13463 |
| TMEM67 | Transmembrane protein 67 | SEQ ID NOS: 13464- |
| | | 13475 |
| TMEM70 | Transmembrane protein 70 | SEQ ID NOS: 13476- |
| | | 13478 |
| TMEM87A | Transmembrane protein 87A | SEQ ID NOS: 13479- |
| | | 13488 |
| TMEM94 | Transmembrane protein 94 | SEQ ID NOS: 13489- |
| | | 13504 |
| TMEM95 | Transmembrane protein 95 | SEQ ID NOS: 13505- |
| | | 13507 |
| TMIGD1 | Transmembrane and immunoglobulin | SEQ ID NOS: 13508- |
| | domain containing 1 | 13509 |
| TMPRSS12 | Transmembrane (C-terminal) protease, | SEQ ID NOS: 13510- |
| | serine 12 | 13511 |
| TMPRSS5 | Transmembrane protease, serine 5 | SEQ ID NOS: 13512- |
| | | 13523 |
| TMUB1 | Transmembrane and ubiquitin-like domain | SEQ ID NOS: 13524- |
| | containing 1 | 13530 |
| TMX2 | Thioredoxin-related transmembrane protein | SEQ ID NOS: 13531- |
| | 2 | 13538 |
| TMX3 | Thioredoxin-related transmembrane protein | SEQ ID NOS: 13539- |
| | 3 | 13546 |
| TNC | Tenascin C | SEQ ID NOS: 13547- |
| | | 13555 |
| TNFAIP6 | Tumor necrosis factor, alpha-induced | SEQ ID NO: 13556 |
| | protein 6 | |
| TNFRSF11A | Tumor necrosis factor receptor superfamily, | SEQ ID NOS: 13557- |
| | member 11a, NFKB activator | 13561 |
| TNFRSF11B | Tumor necrosis factor receptor superfamily, | SEQ ID NOS: 13562- |
| | member 11b | 13563 |
| TNFRSF12A | Tumor necrosis factor receptor superfamily, | SEQ ID NOS: 13564- |
| | member 12A | 13569 |
| TNFRSF14 | Tumor necrosis factor receptor superfamily, | SEQ ID NOS: 13570- |
| | member 14 | 13576 |
| TNFRSF18 | Tumor necrosis factor receptor superfamily, | SEQ ID NOS: 13577- |
| | member 18 | 13580 |
| TNFRSF1A | Tumor necrosis factor receptor superfamily, | SEQ ID NOS: 13581- |
| | member 1A | 13589 |
| TNFRSF1B | Tumor necrosis factor receptor superfamily, | SEQ ID NOS: 13590- |
| | member 1B | 13591 |
| TNFRSF25 | Tumor necrosis factor receptor superfamily, | SEQ ID NOS: 13592- |
| | member 25 | 13603 |
| TNFRSF6B | Tumor necrosis factor receptor superfamily, | SEQ ID NO: 13604 |
| | member 6b, decoy | |
| TNFSF11 | Tumor necrosis factor (ligand) superfamily, | SEQ ID NOS: 13605- |
| | member 11 | 13609 |
| TNFSF12 | Tumor necrosis factor (ligand) superfamily, | SEQ ID NOS: 13610- |
| | member 12 | 13611 |
| TNFSF12- | TNFSF12-TNFSF13 readthrough | SEQ ID NO: 13612 |
| TNFSF13 | | |
| TNFSF15 | Tumor necrosis factor (ligand) superfamily, | SEQ ID NOS: 13613- |
| | member 15 | 13614 |
| TNN | Tenascin N | SEQ ID NOS: 13615- |
| | | 13617 |
| TNR | Tenascin R | SEQ ID NOS: 13618- |
| | | 13620 |
| TNXB | Tenascin XB | SEQ ID NOS: 13621- |
| | | 13627 |
| TOMM7 | Translocase of outer mitochondrial | SEQ ID NOS: 13634- |
| | membrane 7 homolog (yeast) | 13637 |
| TOP1MT | Topoisomerase (DNA) I, mitochondrial | SEQ ID NOS: 13638- |
| | | 13652 |
| TOR1A | Torsin family 1, member A (torsin A) | SEQ ID NO: 13653 |
| TOR1B | Torsin family 1, member B (torsin B) | SEQ ID NOS: 13654- |
| | | 13655 |
| TOR2A | Torsin family 2, member A | SEQ ID NOS: 13656- |
| | | 13662 |
| TOR3A | Torsin family 3, member A | SEQ ID NOS: 13663- |
| | | 13667 |
| TPD52 | Tumor protein D52 | SEQ ID NOS: 13668- |
| | | 13680 |
| TPO | Thyroid peroxidase | SEQ ID NOS: 13681- |
| | | 13691 |
| TPP1 | Tripeptidyl peptidase I | SEQ ID NOS: 13692- |
| | | 13709 |
| TPSAB1 | Tryptase alpha/beta 1 | SEQ ID NOS: 13710- |
| | | 13712 |
| TPSB2 | Tryptase beta 2 (gene/pseudogene) | SEQ ID NOS: 13713- |
| | | 13715 |
| TPSD1 | Tryptase delta 1 | SEQ ID NOS: 13716- |
| | | 13717 |
| TPST1 | Tyrosylprotein sulfotransferase 1 | SEQ ID NOS: 13718- |
| | | 13720 |
| TPST2 | Tyrosylprotein sulfotransferase 2 | SEQ ID NOS: 13721- |
| | | 13729 |
| TRABD2A | TraB domain containing 2A | SEQ ID NOS: 13730- |
| | | 13732 |
| TRABD2B | TraB domain containing 2B | SEQ ID NO: 13733 |
| TREH | Trehalase (brush-border membrane | SEQ ID NOS: 13734- |
| | glycoprotein) | 13736 |
| TREM1 | Triggering receptor expressed on myeloid | SEQ ID NOS: 13737- |
| | cells 1 | 13740 |
| TREM2 | Triggering receptor expressed on myeloid | SEQ ID NOS: 13741- |
| | cells 2 | 13743 |
| TRH | Thyrotropin-releasing hormone | SEQ ID NOS: 13744- |
| | | 13745 |
| TRIM24 | Tripartite motif containing 24 | SEQ ID NOS: 13746- |
| | | 13747 |
| TRIM28 | Tripartite motif containing 28 | SEQ ID NOS: 13748- |
| | | 13753 |
| TRIO | Trio Rho guanine nucleotide exchange | SEQ ID NOS: 13754- |
| | factor | 13760 |
| TRNP1 | TMF1-regulated nuclear protein 1 | SEQ ID NOS: 13761- |
| | | 13762 |
| TSC22D4 | TSC22 domain family, member 4 | SEQ ID NOS: 13763- |
| | | 13766 |
| TSHB | Thyroid stimulating hormone, beta | SEQ ID NOS: 13767- |
| | | 13768 |
| TSHR | Thyroid stimulating hormone receptor | SEQ ID NOS: 13769- |
| | | 13776 |
| TSKU | Tsukushi, small leucine rich proteoglycan | SEQ ID NOS: 13777- |
| | | 13781 |
| TSLP | Thymic stromal lymphopoietin | SEQ ID NOS: 13782- |
| | | 13784 |
| TSPAN3 | Tetraspanin 3 | SEQ ID NOS: 13785- |
| | | 13790 |
| TSPAN31 | Tetraspanin 31 | SEQ ID NOS: 13791- |
| | | 13797 |
| TSPEAR | Thrombospondin-type laminin G domain | SEQ ID NOS: 13798- |
| | and EAR repeats | 13801 |
| TTC13 | Tetratricopeptide repeat domain 13 | SEQ ID NOS: 13802- |
| | | 13808 |
| TTC19 | Tetratricopeptide repeat domain 19 | SEQ ID NOS: 13809- |
| | | 13814 |
| TTC9B | Tetratricopeptide repeat domain 9B | SEQ ID NO: 13815 |
| TTLL11 | Tubulin tyrosine ligase-like family member | SEQ ID NOS: 13816- |
| | 11 | 13820 |
| TTR | Transthyretin | SEQ ID NOS: 13821- |
| | | 13823 |
| TWSG1 | Twisted gastrulation BMP signaling | SEQ ID NOS: 13824- |
| | modulator 1 | 13826 |
| TXNDC12 | Thioredoxin domain containing 12 | SEQ ID NOS: 13827- |
| | (endoplasmic reticulum) | 13829 |
| TXNDC15 | Thioredoxin domain containing 15 | SEQ ID NOS: 13830- |
| | | 13836 |
| TXNDC5 | Thioredoxin domain containing 5 | SEQ ID NOS: 13837- |
| | (endoplasmic reticulum) | 13838 |
| TXNRD2 | Thioredoxin reductase 2 | SEQ ID NOS: 13839- |
| | | 13851 |
| TYRP1 | Tyrosinase-related protein 1 | SEQ ID NOS: 13852- |
| | | 13854 |
| UBAC2 | UBA domain containing 2 | SEQ ID NOS: 13855- |
| | | 13859 |
| UBALD1 | UBA-like domain containing 1 | SEQ ID NOS: 13860- |
| | | 13868 |
| UBAP2 | Ubiquitin associated protein 2 | SEQ ID NOS: 13869- |
| | | 13875 |
| UBXN8 | UBX domain protein 8 | SEQ ID NOS: 13876- |
| | | 13882 |
| UCMA | Upper zone of growth plate and cartilage | SEQ ID NOS: 13883- |
| | matrix associated | 13884 |
| UCN | Urocortin | SEQ ID NO: 13885 |
| UCN2 | Urocortin 2 | SEQ ID NO: 13886 |
| UCN3 | Urocortin 3 | SEQ ID NO: 13887 |
| UGGT2 | UDP-glucose glycoprotein | SEQ ID NOS: 13888- |
| | glucosyltransferase 2 | 13893 |
| UGT1A10 | UDP glucuronosyltransferase 1 family, | SEQ ID NOS: 13894- |
| | polypeptide A10 | 13895 |
| UGT2A1 | UDP glucuronosyltransferase 2 family, | SEQ ID NOS: 13896- |
| | polypeptide A1, complex locus | 13900 |
| UGT2B11 | UDP glucuronosyltransferase 2 family, | SEQ ID NO: 13901 |
| | polypeptide B11 | |
| UGT2B28 | UDP glucuronosyltransferase 2 family, | SEQ ID NOS: 13902- |
| | polypeptide B28 | 13903 |
| UGT2B4 | UDP glucuronosyltransferase 2 family, | SEQ ID NOS: 13904- |
| | polypeptide B4 | 13907 |
| UGT2B7 | UDP glucuronosyltransferase 2 family, | SEQ ID NOS: 13908- |
| | polypeptide B7 | 13911 |
| UGT3A1 | UDP glycosyltransferase 3 family, | SEQ ID NOS: 13912- |
| | polypeptide A1 | 13917 |
| UGT3A2 | UDP glycosyltransferase 3 family, | SEQ ID NOS: 13918- |
| | polypeptide A2 | 13921 |
| UGT8 | UDP glycosyltransferase 8 | SEQ ID NOS: 13922- |
| | | 13924 |
| ULBP3 | UL16 binding protein 3 | SEQ ID NOS: 13925- |
| | | 13926 |
| UMOD | Uromodulin | SEQ ID NOS: 13927- |
| | | 13938 |
| UNC5C | Unc-5 netrin receptor C | SEQ ID NOS: 13939- |
| | | 13943 |
| UPK3B | Uroplakin 3B | SEQ ID NOS: 13944- |
| | | 13946 |
| USP11 | Ubiquitin specific peptidase 11 | SEQ ID NOS: 13947- |
| | | 13950 |
| USP14 | Ubiquitin specific peptidase 14 (tRNA- | SEQ ID NOS: 13951- |
| | guanine transglycosylase) | 13957 |
| USP3 | Ubiquitin specific peptidase 3 | SEQ ID NOS: 13958- |
| | | 13973 |
| UTS2 | Urotensin 2 | SEQ ID NOS: 13984- |
| | | 13986 |
| UTS2B | Urotensin 2B | SEQ ID NOS: 13987- |
| | | 13992 |
| UTY | Ubiquitously transcribed tetratricopeptide | SEQ ID NOS: 13993- |
| | repeat containing, Y-linked | 14005 |
| UXS1 | UDP-glucuronate decarboxylase 1 | SEQ ID NOS: 14006- |
| | | 14013 |
| VASH1 | Vasohibin 1 | SEQ ID NOS: 14014- |
| | | 14016 |
| VCAN | Versican | SEQ ID NOS: 14017- |
| | | 14023 |
| VEGFA | Vascular endothelial growth factor A | SEQ ID NOS: 14024- |
| | | 14049 |
| VEGFB | Vascular endothelial growth factor B | SEQ ID NOS: 14050- |
| | | 14052 |
| VEGFC | Vascular endothelial growth factor C | SEQ ID NO: 14053 |
| VGF | VGF nerve growth factor inducible | SEQ ID NOS: 14055- |
| | | 14057 |
| VIP | Vasoactive intestinal peptide | SEQ ID NOS: 14058- |
| | | 14060 |
| VIPR2 | Vasoactive intestinal peptide receptor 2 | SEQ ID NOS: 14061- |
| | | 14064 |
| VIT | Vitrin | SEQ ID NOS: 14065- |
| | | 14072 |
| VKORC1 | Vitamin K epoxide reductase complex, | SEQ ID NOS: 14073- |
| | subunit 1 | 14080 |
| VLDLR | Very low density lipoprotein receptor | SEQ ID NOS: 14081- |
| | | 14083 |
| VMO1 | Vitelline membrane outer layer 1 homolog | SEQ ID NOS: 14084- |
| | (chicken) | 14087 |
| VNN1 | Vanin 1 | SEQ ID NO: 14088 |
| VNN2 | Vanin 2 | SEQ ID NOS: 14089- |
| | | 14102 |
| VNN3 | Vanin 3 | SEQ ID NOS: 14103- |
| | | 14114 |
| VOPP1 | Vesicular, overexpressed in cancer, | SEQ ID NOS: 14115- |
| | prosurvival protein 1 | 14127 |
| VPREB1 | Pre-B lymphocyte 1 | SEQ ID NOS: 14128- |
| | | 14129 |
| VPREB3 | Pre-B lymphocyte 3 | SEQ ID NOS: 14130- |
| | | 14131 |
| VPS37B | Vacuolar protein sorting 37 homolog B | SEQ ID NOS: 14132- |
| | (S. cerevisiae) | 14134 |
| VPS51 | Vacuolar protein sorting 51 homolog | SEQ ID NOS: 14135- |
| | (S. cerevisiae) | 14146 |
| VSIG1 | V-set and immunoglobulin domain | SEQ ID NOS: 14147- |
| | containing 1 | 14149 |
| VSIG10 | V-set and immunoglobulin domain | SEQ ID NOS: 14150- |
| | containing 10 | 14151 |
| VSTM1 | V-set and transmembrane domain | SEQ ID NOS: 14152- |
| | containing 1 | 14158 |
| VSTM2A | V-set and transmembrane domain | SEQ ID NOS: 14159- |
| | containing 2A | 14162 |
| VSTM2B | V-set and transmembrane domain | SEQ ID NO: 14163 |
| | containing 2B | |
| VSTM2L | V-set and transmembrane domain | SEQ ID NOS: 14164- |
| | containing 2 like | 14166 |
| VSTM4 | V-set and transmembrane domain | SEQ ID NOS: 14167- |
| | containing 4 | 14168 |
| VTN | Vitronectin | SEQ ID NOS: 14169- |
| | | 14170 |
| VWA1 | Von Willebrand factor A domain containing | SEQ ID NOS: 14171- |
| | 1 | 14174 |
| VWA2 | Von Willebrand factor A domain containing | SEQ ID NOS: 14175- |
| | 2 | 14176 |
| VWA5B2 | Von Willebrand factor A domain containing | SEQ ID NOS: 14177- |
| | 5B2 | 14178 |
| VWA7 | Von Willebrand factor A domain containing | SEQ ID NO: 14179 |
| | 7 | |
| VWC2 | Von Willebrand factor C domain containing | SEQ ID NO: 14180 |
| | 2 | |
| VWC2L | Von Willebrand factor C domain containing | SEQ ID NOS: 14181- |
| | protein 2-like | 14182 |
| VWCE | Von Willebrand factor C and EGF domains | SEQ ID NOS: 14183- |
| | | 14187 |
| VWDE | Von Willebrand factor D and EGF domains | SEQ ID NOS: 14188- |
| | | 14193 |
| VWF | Von Willebrand factor | SEQ ID NOS: 14194- |
| | | 14196 |
| WDR25 | WD repeat domain 25 | SEQ ID NOS: 14197- |
| | | 14203 |
| WDR81 | WD repeat domain 81 | SEQ ID NOS: 14204- |
| | | 14213 |
| WDR90 | WD repeat domain 90 | SEQ ID NOS: 14214- |
| | | 14221 |
| WFDC1 | WAP four-disulfide core domain 1 | SEQ ID NOS: 14222- |
| | | 14224 |
| WFDC10A | WAP four-disulfide core domain 10A | SEQ ID NO: 14225 |
| WFDC10B | WAP four-disulfide core domain 10B | SEQ ID NOS: 14226- |
| | | 14227 |
| WFDC11 | WAP four-disulfide core domain 11 | SEQ ID NOS: 14228- |
| | | 14230 |
| WFDC12 | WAP four-disulfide core domain 12 | SEQ ID NO: 14231 |
| WFDC13 | WAP four-disulfide core domain 13 | SEQ ID NO: 14232 |
| WFDC2 | WAP four-disulfide core domain 2 | SEQ ID NOS: 14233- |
| | | 14237 |
| WFDC3 | WAP four-disulfide core domain 3 | SEQ ID NOS: 14238- |
| | | 14241 |
| WFDC5 | WAP four-disulfide core domain 5 | SEQ ID NOS: 14242- |
| | | 14243 |
| WFDC6 | WAP four-disulfide core domain 6 | SEQ ID NOS: 14244- |
| | | 14245 |
| WFDC8 | WAP four-disulfide core domain 8 | SEQ ID NOS: 14246- |
| | | 14247 |
| WFIKKN1 | WAP, follistatin/kazal, immunoglobulin, | SEQ ID NO: 14248 |
| | kunitz and netrin domain containing 1 | |
| WFIKKN2 | WAP, follistatin/kazal, immunoglobulin, | SEQ ID NOS: 14249- |
| | kunitz and netrin domain containing 2 | 14250 |
| WIF1 | WNT inhibitory factor 1 | SEQ ID NOS: 14255- |
| | | 14257 |
| WISP1 | WNT1 inducible signaling pathway protein | SEQ ID NOS: 14258- |
| | 1 | 14262 |
| WISP2 | WNT1 inducible signaling pathway protein | SEQ ID NOS: 14263- |
| | 2 | 14265 |
| WISP3 | WNT1 inducible signaling pathway protein | SEQ ID NOS: 14266- |
| | 3 | 14273 |
| WNK1 | WNK lysine deficient protein kinase 1 | SEQ ID NOS: 14274- |
| | | 14287 |
| WNT1 | Wingless-type MMTV integration site | SEQ ID NOS: 14288- |
| | family, member 1 | 14289 |
| WNT10B | Wingless-type MMTV integration site | SEQ ID NOS: 14290- |
| | family, member 10B | 14294 |
| WNT11 | Wingless-type MMTV integration site | SEQ ID NOS: 14295- |
| | family, member 11 | 14297 |
| WNT16 | Wingless-type MMTV integration site | SEQ ID NOS: 14298- |
| | family, member 16 | 14299 |
| WNT2 | Wingless-type MMTV integration site | SEQ ID NOS: 14300- |
| | family member 2 | 14302 |
| WNT3 | Wingless-type MMTV integration site | SEQ ID NO: 14303 |
| | family, member 3 | |
| WNT3A | Wingless-type MMTV integration site | SEQ ID NO: 14304 |
| | family, member 3A | |
| WNT5A | Wingless-type MMTV integration site | SEQ ID NOS: 14305- |
| | family, member 5A | 14308 |
| WNT5B | Wingless-type MMTV integration site | SEQ ID NOS: 14309- |
| | family, member 5B | 14315 |
| WNT6 | Wingless-type MMTV integration site | SEQ ID NO: 14316 |
| | family, member 6 | |
| WNT7A | Wingless-type MMTV integration site | SEQ ID NO: 14317 |
| | family, member 7A | |
| WNT7B | Wingless-type MMTV integration site | SEQ ID NOS: 14318- |
| | family, member 7B | 14322 |
| WNT8A | Wingless-type MMTV integration site | SEQ ID NOS: 14323- |
| | family, member 8A | 14326 |
| WNT8B | Wingless-type MMTV integration site | SEQ ID NO: 14327 |
| | family, member 8B | |
| WNT9A | Wingless-type MMTV integration site | SEQ ID NO: 14328 |
| | family, member 9A | |
| WNT9B | Wingless-type MMTV integration site | SEQ ID NOS: 14329- |
| | family, member 9B | 14331 |
| WSB1 | WD repeat and SOCS box containing 1 | SEQ ID NOS: 14332- |
| | | 14341 |
| WSCD1 | WSC domain containing 1 | SEQ ID NOS: 14342- |
| | | 14351 |
| WSCD2 | WSC domain containing 2 | SEQ ID NOS: 14352- |
| | | 14355 |
| XCL1 | Chemokine (C motif) ligand 1 | SEQ ID NO: 14356 |
| XCL2 | Chemokine (C motif) ligand 2 | SEQ ID NO: 14357 |
| XPNPEP2 | X-prolyl aminopeptidase (aminopeptidase | SEQ ID NOS: 14358- |
| | P) 2, membrane-bound | 14359 |
| XXbac- | | SEQ ID NOS: 679- 680 |
| BPG116M5.17 | | |
| XXbac- | | SEQ ID NO: 681 |
| BPG181M17.5 | | |
| XXbac- | | SEQ ID NO: 682 |
| BPG32J3.20 | | |
| XXYLT1 | Xyloside xylosyltransferase 1 | SEQ ID NOS: 14360- |
| | | 14365 |
| XYLT1 | Xylosyltransferase I | SEQ ID NO: 14366 |
| XYLT2 | Xylosyltransferase II | SEQ ID NOS: 14367- |
| | | 14372 |
| ZFYVE21 | Zinc finger, FYVE domain containing 21 | SEQ ID NOS: 14373- |
| | | 14377 |
| ZG16 | Zymogen granule protein 16 | SEQ ID NO: 14378 |
| ZG16B | Zymogen granule protein 16B | SEQ ID NOS: 14379- |
| | | 14382 |
| ZIC4 | Zic family member 4 | SEQ ID NOS: 14383- |
| | | 14391 |
| ZNF207 | Zinc finger protein 207 | SEQ ID NOS: 14392- |
| | | 14402 |
| ZNF26 | Zinc finger protein 26 | SEQ ID NOS: 14403- |
| | | 14406 |
| ZNF34 | Zinc finger protein 34 | SEQ ID NOS: 14407- |
| | | 14410 |
| ZNF419 | Zinc finger protein 419 | SEQIDNOS: 14411- |
| | | 14425 |
| ZNF433 | Zinc finger protein 433 | SEQ ID NOS: 14426- |
| | | 14435 |
| ZNF449 | Zinc finger protein 449 | SEQ ID NOS: 14436- |
| | | 14437 |
| ZNF488 | Zinc finger protein 488 | SEQ ID NOS: 14438- |
| | | 14439 |
| ZNF511 | Zinc finger protein 511 | SEQ ID NOS: 14440- |
| | | 14441 |
| ZNF570 | Zinc finger protein 570 | SEQ ID NOS: 14442- |
| | | 14447 |
| ZNF691 | Zinc finger protein 691 | SEQ ID NOS: 14448- |
| | | 14455 |
| ZNF98 | Zinc finger protein 98 | SEQ ID NOS: 14456- |
| | | 14459 |
| ZPBP | Zona pellucida binding protein | SEQ ID NOS: 14460- |
| | | 14463 |
| ZPBP2 | Zona pellucida binding protein 2 | SEQ ID NOS: 14464- |
| | | 14467 |
| ZSCAN29 | Zinc finger and SCAN domain containing | SEQ ID NOS: 14468- |
| | 29 | 14474 |

### Cas-Clover

The disclosure provides a composition comprising a guide RNA and a fusion protein or a sequence encoding the fusion protein wherein the fusion protein comprises a dCas9 and a Clo051 endonuclease or a nuclease domain thereof.

### Small Cas9 (SaCas9)

The disclosure provides compositions comprising a small, Cas9 (Cas9) operatively-linked to an effector. In certain embodiments, the disclosure provides a fusion protein comprising, consisting essentially of or consisting of a DNA localization component and an effector molecule, wherein the effector comprises a small, Cas9 (Cas9). In certain embodiments, a small Cas9 construct of the disclosure may comprise an effector comprising a type IIS endonuclease.

Amino acid sequence of Staphylococcus aureus Cas9 with an active catalytic site.

```
(SEQ ID NO: 18052)
      1 mkrnyilgld igitsvgygi idyetrdvid agvrlfkean
          vennegrrsk rgarrlkrrr
    61 rhriqrvkkl lfdynlltdh selsginpye arvkglsqkl
          seeefsaall hlakrrgvhn
  121 vneveedtgn elstkeqisr nskaleekyv aelqlerlkk
          dgevrgsinr fktsdyvkea
  181 kqllkvqkay hqldqsfidt yidlletrrt yyegpgegsp
          fgwkdikewy emlmghctyf
  241 peelrsvkya ynadlynaln dlnnlvitrd enekleyyek
          fqiienvfkq kkkptlkqia
  301 keilvneedi kgyrvtstgk peftnlkvyh dikditarke
          iienaelldq iakiltiyqs
  361 sediqeeltn lnseltqeei eqisnlkgyt gthnlslkai
          nlildelwht ndnqiaifnr
  421 lklvpkkvd1 sqqkeipttl vddfilspvv krsfiqsikv
          inaiikkygl pndiiielar
  481 eknskdaqkm inemqkrnrq tnerieeiir ttgkenakyl
          iekiklhdmq egkclyslea
  541 ipledllnnp fnyevdhiip rsysfdnsfn nkvlvkqeen
          skkgnrtpfq ylsssdskis
  601 yetfkkhiln lakgkgrisk tkkeylleer dinrfsvqkd
          finrnlvdtr yatrglmnll
  661 rsyfrvnnld vkvksinggf tsflrrkwkf kkernkgykh
          haedaliian adfifkewkk
  721 ldkakkvmen qmfeekqaes mpeieteqey keifitphqi
          khikdfkdyk yshrvdkkpn
  781 relindtlys trkddkgntl ivnnlnglyd kdndklkkli
          nkspekllmy hhdpqtyqkl
  841 klimeqygde knplykyyee tgnyltkysk kdngpvikki
          kyygnklnah lditddypns
  901 rnkvvklslk pyrfdvyldn gvykfvtvkn ldvikkenyy
          evnskcyeea kklkkisnqa
  961 efiasfynnd likingelyr vigvnndlln rievnmidit
          yreylenmnd krppriikti
1021 asktqsikky stdilgnlye vkskkhpqii kkg
```

Inactivated, Small Cas9 (dSaCas9)

The disclosure provides compositions comprising an inactivated, small, Cas9 (dSaCas9) operatively-linked to an effector. In certain embodiments, the disclosure provides a fusion protein comprising, consisting essentially of or consisting of a DNA localization component and an effector molecule, wherein the effector comprises a small, inactivated Cas9 (dSaCas9). In certain embodiments, a small, inactivated Cas9 (dSaCas9) construct of the disclosure may comprise an effector comprising a type IIS endonuclease.

dSaCas9 Sequence: D10A and N580A mutations (bold, capitalized, and underlined) inactivate the catalytic site.

```
(SEQ ID NO: 18053)
      1 mkrnyilglA igitsvgygi idyetrdvid agvrlfkean
          vennegrrsk rgarrlkrrr
    61 rhriqrvkkl lfdynlltdh selsginpye arvkglsqkl
          seeefsaall hlakrrgvhn
  121 vneveedtgn elstkeqisr nskaleekyv aelqlerlkk
          dgevrgsinr fktsdyvkea
  181 kqllkvqkay hqldqsfidt yidlletrrt yyegpgegsp
          fgwkdikewy emlmghctyf
  241 peelrsvkya ynadlynaln dlnnlvitrd enekleyyek
          fqiienvfkq kkkptlkqia
  301 keilvneedi kgyrvtstgk peftnlkvyh dikditarke
          iienaelldq iakiltiyqs
  361 sediqeeltn lnseltqeei eqisnlkgyt gthnlslkai
          nlildelwht ndnqiaifnr
  421 lklvpkkvd1 sqqkeipttl vddfilspvv krsfiqsikv
          inaiikkygl pndiiielar
  481 eknskdaqkm inemqkrnrq tnerieeiir ttgkenakyl
          iekiklhdmq egkclyslea
  541 ipledllnnp fnyevdhiip rsysfdnsfn nkvlvkqeeA
          skkgnrtpfq ylsssdskis
  601 yetfkkhiln lakgkgrisk tkkeylleer dinrfsvqkd
          finrnlvdtr yatrglmnll
  661 rsyfrvnnld vkvksinggf tsflrrkwkf kkernkgykh
          haedaliian adfifkewkk
  721 ldkakkvmen qmfeekqaes mpeieteqey keifitphqi
          khikdfkdyk yshrvdkkpn
  781 relindtlys trkddkgntl ivnnlnglyd kdndklkkli
          nkspekllmy hhdpqtyqkl
  841 klimeqygde knplykyyee tgnyltkysk kdngpvikki
          kyygnklnah lditddypns
  901 rnkvvklslk pyrfdvyldn gvykfvtvkn ldvikkenyy
          evnskcyeea kklkkisnqa
  961 efiasfynnd likingelyr vigvnndlln rievnmidit
          yreylenmnd krppriikti
1021 asktqsikky stdilgnlye vkskkhpqii kkg
```

Inactivated Cas9 (dCas9)

The disclosure provides compositions comprising an inactivated Cas9 (dCas9) operatively-linked to an effector. In certain embodiments, the disclosure provides a fusion protein comprising, consisting essentially of or consisting of a DNA localization component and an effector molecule, wherein the effector comprises an inactivated Cas9 (dCas9). In certain embodiments, an inactivated Cas9 (dCas9) construct of the disclosure may comprise an effector comprising a type IIS endonuclease.

In certain embodiments, the dCas9 of the disclosure comprises a dCas9 isolated or derived from Staphyloccocus pyogenes. In certain embodiments, the dCas9 comprises a dCas9 with substitutions at positions 10 and 840 of the amino acid sequence of the dCas9 which inactivate the catalytic site. In certain embodiments, these substitutions are D10A and H840A. In certain embodiments, the amino acid sequence of the dCas9 comprises the sequence of:

```
(SEQ ID NO: 18054)
      1 XDKKYSIGLA IGTNSVGWAV ITDEYKVPSK KFKVLGNTDR
          HSIKKNLIGA LLFDSGETAE
    61 ATRLKRTARR RYTRRKNRIC YLQEIFSNEM AKVDDSFFHR
          LEESFLVEED KKHERHPIFG
  121 NIVDEVAYHE KYPTIYHLRK KLVDSTDKAD LRLIYLALAH
          MIKFRGHFLI EGDLNPDNSD
  181 VDKLFIQLVQ TYNQLFEENP INASGVDAKA ILSARLSKSR
          RLENLIAQLP GEKKNGLFGN
  241 LIALSLGLTP NFKSNFDLAE DAKLQLSKDT YDDDLDNLLA
          QIGDQYADLF LAAKNLSDAI
  301 LLSDILRVNT EITKAPLSAS MIKRYDEHHQ DLTLLKALVR
          QQLPEKYKEI FFDQSKNGYA
  361 GYIDGGASQE EFYKFIKPIL EKMDGTEELL VKLNREDLLR
          KQRTFDNGSI PHQIHLGELH
  421 AILRRQEDFY PFLKDNREKI EKILTFRIPY YVGPLARGNS
          RFAWMTRKSE ETITPWNFEE
  481 VVDKGASAQS FIERMTNFDK NLPNEKVLPK HSLLYEYFTV
          YNELTKVKYV TEGMRKPAFL
  541 SGEQKKAIVD LLFKTNRKVT VKQLKEDYFK KIECFDSVEI
          SGVEDRFNAS LGTYHDLLKI
  601 IKDKDFLDNE ENEDILEDIV LTLTLFEDRE MIEERLKTYA
          HLFDDKVMKQ LKRRRYTGWG
  661 RLSRKLINGI RDKQSGKTIL DFLKSDGFAN RNFMQLIHDD
          SLTFKEDIQK AQVSGQGDSL
  721 HEHIANLAGS PAIKKGILQT VKVVDELVKV MGRHKPENIV
          IEMARENQTT QKGQKNSRER
  781 MKRIEEGIKE LGSQILKEHP VENTQLQNEK LYLYYLQNGR
          DMYVDQELDI NRLSDYDVDA
  841 IVPQSFLKDD SIDNKVLTRS DKNRGKSDNV PSEEVVKKMK
          NYWRQLLNAK LITQRKFDNL
  901 TKAERGGLSE LDKAGFIKRQ LVETRQITKH VAQILDSRMN
          TKYDENDKLI REVKVITLKS
  961 KLVSDFRKDF QFYKVREINN YHHAHDAYLN AVVGTALIKK
          YPKLESEFVY GDYKVYDVRK
1021 MIAKSEQEIG KATAKYFFYS NIMNFFKTEI TLANGEIRKR
          PLIETNGETG EIVWDKGRDF
1081 ATVRKVLSMP QVNIVKKTEV QTGGFSKESI LPKRNSDKLI
          ARKKDWDPKK YGGFDSPTVA
1141 YSVLVVAKVE KGKSKKLKSV KELLGITIME RSSFEKNPID
          FLEAKGYKEV KKDLIIKLPK
1201 YSLFELENGR KRMLASAGEL QKGNELALPS KYVNFLYLAS
          HYEKLKGSPE DNEQKQLFVE
1261 QHKHYLDEII EQISEFSKRV ILADANLDKV LSAYNKHRDK
          PIREQAENII HLFTLTNLGA
1321 PAAFKYFDTT IDRKRYTSTK EVLDATLIHQ SITGLYETRI
          DLSQLGGD.
```

In certain embodiments, the amino acid sequence of the dCas9 comprises the sequence of:

```
(SEQ ID NO: 18055)
      1 MDKKYSIGLA IGTNSVGWAV ITDEYKVPSK KFKVLGNTDR
          HSIKKNLIGA LLFDSGETAE
    61 ATRLKRTARR RYTRRKNRIC YLQEIFSNEM AKVDDSFFHR
          LEESFLVEED KKHERHPIFG
  121 NIVDEVAYHE KYPTIYHLRK KLVDSTDKAD LRLIYLALAH
          MIKFRGHFLI EGDLNPDNSD
  181 VDKLFIQLVQ TYNQLFEENP INASGVDAKA ILSARLSKSR
          RLENLIAQLP GEKKNGLFGN
  241 LIALSLGLTP NFKSNFDLAE DAKLQLSKDT YDDDLDNLLA
          QIGDQYADLF LAAKNLSDAI
  301 LLSDILRVNT EITKAPLSAS MIKRYDEHHQ DLTLLKALVR
          QQLPEKYKEI FFDQSKNGYA
  361 GYIDGGASQE EFYKFIKPIL EKMDGTEELL VKLNREDLLR
          KQRTFDNGSI PHQIHLGELH
  421 AILRRQEDFY PFLKDNREKI EKILTFRIPY YVGPLARGNS
          RFAWMTRKSE ETITPWNFEE
  481 VVDKGASAQS FIERMTNFDK NLPNEKVLPK HSLLYEYFTV
          YNELTKVKYV TEGMRKPAFL
  541 SGEQKKAIVD LLFKTNRKVT VKQLKEDYFK KIECFDSVEI
          SGVEDRFNAS LGTYHDLLKI
  601 IKDKDFLDNE ENEDILEDIV LTLTLFEDRE MIEERLKTYA
          HLFDDKVMKQ LKRRRYTGWG
  661 RLSRKLINGI RDKQSGKTIL DFLKSDGFAN RNFMQLIHDD
          SLTFKEDIQK AQVSGQGDSL
  721 HEHIANLAGS PAIKKGILQT VKVVDELVKV MGRHKPENIV
          IEMARENQTT QKGQKNSRER
  781 MKRIEEGIKE LGSQILKEHP VENTQLQNEK LYLYYLQNGR
          DMYVDQELDI NRLSDYDVDA
  841 IVPQSFLKDD SIDNKVLTRS DKNRGKSDNV PSEEVVKKMK
          NYWRQLLNAK LITQRKFDNL
  901 TKAERGGLSE LDKAGFIKRQ LVETRQITKH VAQILDSRMN
          TKYDENDKLI REVKVITLKS
  961 KLVSDFRKDF QFYKVREINN YHHAHDAYLN AVVGTALIKK
          YPKLESEFVY GDYKVYDVRK
1021 MIAKSEQEIG KATAKYFFYS NIMNFFKTEI TLANGEIRKR
          PLIETNGETG EIVWDKGRDF
1081 ATVRKVLSMP QVNIVKKTEV QTGGFSKESI LPKRNSDKLI
          ARKKDWDPKK YGGFDSPTVA
1141 YSVLVVAKVE KGKSKKLKSV KELLGITIME RSSFEKNPID
          FLEAKGYKEV KKDLIIKLPK
1201 YSLFELENGR KRMLASAGEL QKGNELALPS KYVNFLYLAS
          HYEKLKGSPE DNEQKQLFVE
1261 QHKHYLDEII EQISEFSKRV ILADANLDKV LSAYNKHRDK
          PIREQAENII HLFTLTNLGA
1321 PAAFKYFDTT IDRKRYTSTK EVLDATLIHQ SITGLYETRI
          DLSQLGGD.
```

### Clo051 Endonuclease

An exemplary Clo051 nuclease domain may comprise, consist essentially of or consist of, the amino acid sequence of:

(SEQ ID NO: 18056)
EGIKSNISLLKDELRGQISHISHEYLSLIDLAFDSKQNRLFEMKVLE
LLVNEYGFKGRHLGGSRKPDGIVYSTTLEDNFGIIVDTKAYSEGYSL
PISQADEMERYVRENSNRDEEVNPNKWWENFSEEVKKYYFVFISGSF
KGKFEEQLRRLSMTTGVNGSAVNVVNLLLGAEKIRSGEMTIEELERA
MFNNSEFILKY.

### Cas-Clover Fusion Protein

In certain embodiments, an exemplary dCas9-Clo051 fusion protein (embodiment 1) may comprise, consist essentially of or consist of, the amino acid sequence of (Clo051 sequence underlined, linker bold italics, dCas9 sequence (Streptoccocus pyogenes) in italics):

(SEQ ID NO: 18057)
MAPKKKRKVEGIKSNISLLKDELRGQISHISHEYLSLIDLAFDSK
QNRLFEMKVLELLVNEYGFKGRHLGGSRKPDGIVYSTTLEDNFGI
IVDTKAYSEGYSLPISQADEMERYVRENSNRDEEVNPNKWWENFS
EEVKKYYFVFISGSFKGKFEEQLRRLSMTTGVNGSAVNVVNLLLG
AEKIRSGEMTIEELERAMFNNSEFILKY [symbol] DKKYSIGLAIGT
NSVGWAVITDEYKVPSKKFKVLGNTDRHSIKKNLIGALLFDSGET
AEATRLKRTARRRYTRRKNRICYLQEIFSNEMAKVDDSFEHRLEE
SELVEEDKKHERHPIFGNIVDEVAYHEKYPTIYHLRKKLVDSTDK
ADLRLIYLALAHMIKFRGHFLIEGDLNPDNSDVDKLFIQLVQTYN
QLFEENPINASGVDAKAILSARLSKSRRLENLIAQLPGEKKNGLF
GNLIALSLGLTPNEKSNFDLAEDAKLQLSKDTYDDDLDNLLAQIG
DQYADLFLAAKNLSDAILLSDILRVNTEITKAPLSASMIKRYDEH
HQDLTLLKALVRQQLPEKYKEIFFDQSKNGYAGYIDGGASQEEFY
KFIKPILEKMDGTEELLVKLNREDLLRKQRTEDNGSIPHQIHLGE
LHAILRRQEDFYPFLKDNREKIEKILTFRIPYYVGPLARGNSRFA
WMTRKSEETITPWNFEEVVDKGASAQSFIERMTNFDKNLPNEKVL
PKHSLLYEYFTVYNELTKVKYVTEGMRKPAFLSGEQKKAIVDLLF
KTNRKVTVKQLKEDYFKKIECFDSVEISGVEDRFNASLGTYHDLL
KIIKDKDFLDNEENEDILEDIVLTLTLFEDREMIEERLKTYAHLF
DDKVMKQLKRRRYTGWGRLSRKLINGIRDKQSGKTILDFLKSDGF
ANRNFMQLIHDDSLTFKEDIQKAQVSGQGDSLHEHIANLAGSPAI
KKGILQTVKVVDELVKVMGRHKPENIVIEMARENQTTQKGQKNSR
ERMKRIEEGIKELGSQILKEHPVENTQLQNEKLYLYYLQNGRDMY
VDQELDINRLSDYDVDAIVPQSFLKDDSIDNKVLTRSDKNRGKSD
NVPSEEVVKKMKNYWRQLLNAKLITQRKEDNLTKAERGGLSELDK
AGFIKRQLVETRQITKHVAQILDSRMNTKYDENDKLIREVKVITL
KSKLVSDFRKDFQFYKVREINNYHHAHDAYLNAVVGTALIKKYPK
LESEFVYGDYKVYDVRKMIAKSEQEIGKATAKYFFYSNIMNFFKT
EITLANGEIRKRPLIETNGETGEIVWDKGRDFATVRKVLSMPQVN
IVKKTEVQTGGESKESILPKRNSDKLIARKKDWDPKKYGGFDSPT
VAYSVLVVAKVEKGKSKKLKSVKELLGITIMERSSFEKNPIDFLE
AKGYKEVKKDLIIKLPKYSLFELENGRKRMLASAGELQKGNELAL
PSKYVNFLYLASHYEKLKGSPEDNEQKQLFVEQHKHYLDEIIEQI
SEFSKRVILADANLDKVLSAYNKHRDKPIREQAENIIHLFTLTNL
GAPAAFKYFDTTIDRKRYTSTKEVLDATLIHQSITGLYETRIDLS
QLGGDGSPKKKRKVSS.

In certain embodiments, an exemplary dCas9-Clo051 fusion protein (embodiment 1) may comprise, consist essentially of or consist of, the nucleic acid sequence of (dCas9 sequence derived from Streptoccocus pyogenes):

```
(SEQ ID NO: 18054)
      1 atggcaccaa agaagaaaag aaaagtggag ggcatcaagt caaacatcag cctgctgaaa
    61 gacgaactgc ggggacagat tagtcacatc agtcacgagt acctgtcact gattgatctg
  121 gccttcgaca gcaagcagaa tagactgttt gagatgaaag tgctggaact gctggtcaac
  181 gagtatggct tcaagggcag acatctgggc gggtctagga aacctgacgg catcgtgtac
  241 agtaccacac tggaagacaa cttcggaatc attgtcgata ccaaggctta ttccgagggc
  301 tactctctgc caattagtca ggcagatgag atggaaaggt acgtgcgcga aaactcaaat
  361 agggacgagg aagtcaaccc caataagtgg tgggagaatt tcagcgagga agtgaagaaa
  421 tactacttcg tctttatctc aggcagcttc aaagggaagt ttgaggaaca gctgcggaga
  481 ctgtccatga ctaccggggt gaacggatct gctgtcaacg tggtcaatct gctgctgggc
  541 gcagaaaaga tcaggtccgg ggagatgaca attgaggaac tggaacgcgc catgttcaac
  601 aattctgagt ttatcctgaa gtatggaggc gggggaagcg ataagaaata ctccatcgga
  661 ctggccattg gcaccaattc cgtgggctgg gctgtcatca cagacgagta caaggtgcca
  721 agcaagaagt tcaaggtcct ggggaacacc gatcgccaca gtatcaagaa aaatctgatt
  781 ggagccctgc tgttcgactc aggcgagact gctgaagcaa cccgactgaa gcggactgct
  841 aggcgccgat atacccggag aaaaaatcgg atctgctacc tgcaggaaat tttcagcaac
  901 gagatggcca aggtggacga tagtttcttt caccgcctgg aggaatcatt cctggtggag
  961 gaagataaga aacacgagcg gcatcccatc tttggcaaca ttgtggacga agtcgcttat
1021 cacgagaagt accctactat ctatcatctg aggaagaaac tggtggactc caccgataag
1081 gcagacctgc gcctgatcta tctggccctg gctcacatga tcaagttccg ggggcatttt
1141 ctgatcgagg gagatctgaa ccctgacaat tctgatgtgg acaagctgtt catccagctg
1201 gtccagacat acaatcagct gtttgaggaa aacccaatta atgcctcagg cgtggacgca
1261 aaggccatcc tgagcgccag actgtccaaa tctaggcgcc tggaaaacct gatcgctcag
1321 ctgccaggag agaagaaaaa cggcctgttt gggaatctga ttgcactgtc cctgggcctg
1381 acacccaact tcaagtctaa ttttgatctg gccgaggacg ctaagctgca gctgtccaaa
1441 gacacttatg acgatgacct ggataacctg ctggctcaga tcggcgatca gtacgcagac
1501 ctgttcctgg ccgctaagaa tctgagtgac gccatcctgc tgtcagatat tctgcgcgtg
1561 aacacagaga ttactaaggc cccactgagt gcttcaatga tcaaaagata tgacgagcac
1621 catcaggatc tgaccctgct gaaggctctg gtgaggcagc agctgcccga gaaatacaag
1681 gaaatcttct ttgatcagag caagaatgga tacgccggct atattgacgg cggggcttcc
1741 caggaggagt tctacaagtt catcaagccc attctggaaa agatggacgg caccgaggaa
1801 ctgctggtga agctgaatcg ggaggacctg ctgagaaaac agaggacatt tgataacgga
1861 agcatccctc accagattca tctgggcgaa ctgcacgcca tcctgcgacg gcaggaggac
1921 ttctacccat ttctgaagga taaccgcgag aaaatcgaaa agatcctgac cttcagaatc
1981 ccctactatg tggggcctct ggcacgggga aatagtagat ttgcctggat gacaagaaag
2041 tcagaggaaa ctatcacccc ctggaacttc gaggaagtgg tcgataaagg cgctagcgca
2101 cagtccttca ttgaaaggat gacaaatttt gacaagaacc tgccaaatga gaaggtgctg
2161 cccaaacaca gcctgctgta cgaatatttc acagtgtata acgagctgac taaagtgaag
2221 tacgtcaccg aagggatgcg caagcccgca ttcctgtccg gagagcagaa gaaagccatc
2281 gtggacctgc tgtttaagac aaatcggaaa gtgactgtca aacagctgaa ggaagactat
2341 ttcaagaaaa ttgagtgttt cgattcagtg gaaatcagcg gcgtcgagga caggtttaac
2401 gcctccctgg ggacctacca cgatctgctg aagatcatca aggataagga cttcctggac
2461 aacgaggaaa atgaggacat cctggaggac attgtgctga cactgactct gtttgaggat
2521 cgcgaaatga tcgaggaacg actgaagact tatgcccatc tgttcgatga caaagtgatg
2581 aagcagctga aaagaaggcg ctacaccgga tggggacgcc tgagccgaaa actgatcaat
2641 gggattagag acaagcagag cggaaaaact atcctggact ttctgaagtc cgatggcttc
2701 gccaacagga acttcatgca gctgattcac gatgactctc tgaccttcaa ggaggacatc
2761 cagaaagcac aggtgtctgg ccagggggac agtctgcacg agcatatcgc aaacctggcc
2821 ggcagccccg ccatcaagaa agggattctg cagaccgtga aggtggtgga cgaactggtc
2881 aaggtcatgg gacgacacaa acctgagaac atcgtgattg agatggcccg cgaaaatcag
2941 acaactcaga agggccagaa aaacagtcga gaacggatga agagaatcga ggaaggcatc
3001 aaggagctgg ggtcacagat cctgaaggag catcctgtgg aaaacactca gctgcagaat
3061 gagaaactgt atctgtacta tctgcagaat ggacgggata tgtacgtgga ccaggagctg
3121 gatattaaca gactgagtga ttatgacgtg gatgccatcg tccctcagag cttcctgaag
3181 gatgactcca ttgacaacaa ggtgctgacc aggtccgaca agaaccgcgg caaatcagat
3241 aatgtgccaa gcgaggaagt ggtcaagaaa atgaagaact actggaggca gctgctgaat
3301 gccaagctga tcacacagcg gaaatttgat aacctgacta aggcagaaag aggaggcctg
3361 tctgagctgg acaaggccgg cttcatcaag cggcagctgg tggagacaag acagatcact
3421 aagcacgtcg ctcagattct ggatagcaga atgaacacaa agtacgatga aaacgacaag
3481 ctgatcaggg aggtgaaagt cattactctg aaatccaagc tggtgtctga ctttagaaag
3541 gatttccagt tttataaagt cagggagatc aacaactacc accatgctca tgacgcatac
3601 ctgaacgcag tggtcgggac cgccctgatt aagaaatacc ccaagctgga gtccgagttc
3661 gtgtacggag actataaagt gtacgatgtc cggaagatga tcgccaaatc tgagcaggaa
3721 attggcaagg ccaccgctaa gtatttcttt tacagtaaca tcatgaattt ctttaagacc
3781 gaaatcacac tggcaaatgg ggagatcaga aaaaggcctc tgattgagac caacggggag
3841 acaggagaaa tcgtgtggga caagggaagg gattttgcta ccgtgcgcaa agtcctgtcc
3901 atgccccaag tgaatattgt caagaaaact gaagtgcaga ccgggggatt ctctaaggag
3961 agtattctgc ctaagcgaaa ctctgataaa ctgatcgccc ggaagaaaga ctgggacccc
4021 aagaagtatg gcgggttcga ctctccaaca gtggcttaca gtgtcctggt ggtcgcaaag
4081 gtggaaaagg ggaagtccaa gaaactgaag tctgtcaaag agctgctggg aatcactatt
4141 atggaacgca gctccttcga gaagaatcct atcgattttc tggaagccaa gggctataaa
4201 gaggtgaaga aagacctgat cattaagctg ccaaaatact cactgtttga gctggaaaac
4261 ggacgaaagc gaatgctggc aagcgccgga gaactgcaga agggcaatga gctggccctg
4321 ccctccaaat acgtgaactt cctgtatctg gctagccact acgagaaact gaaggggtcc
4381 cctgaggata acgaacagaa gcagctgttt gtggagcagc acaaacatta tctggacgag
4441 atcattgaac agatttcaga gttcagcaag agagtgatcc tggctgacgc aaatctggat
4501 aaagtcctga gcgcatacaa caagcaccga gacaaaccaa tccgggagca ggccgaaaat
4561 atcattcatc tgttcaccct gacaaacctg ggcgcccctg cagccttcaa gtattttgac
4621 accacaatcg atcggaagag atacacttct accaaagagg tgctggatgc taccctgatc
4681 caccagagta ttaccggcct gtatgagaca cgcatcgacc tgtcacagct gggaggcgat
4741 gggagcccca agaaaaagcg gaaggtgtct agttaa
```

In certain embodiments, the nucleic acid sequence encoding a dCas9-Clo051 fusion protein (embodiment 1) of the disclosure may comprise a DNA. In certain embodiments, the nucleic acid sequence encoding a dCas9-Clo051 fusion protein (embodiment 1) of the disclosure may comprise an RNA.

In certain embodiments, an exemplary dCas9-Clo051 fusion protein (embodiment 2) may comprise, consist essentially of or consist of, the amino acid sequence of (Clo051 sequence underlined, linker bold italics, dCas9 sequence (Streptoccocus pyogenes) in italics):

```
(SEQ ID NO: 18059)
      1 MPKKKRKVEG IKSNISLLKD ELRGQISHIS HEYLSLIDLA FDSKQNRLFE MKVLELLVNE
    61 YGFKGRHLGG SRKPDGIVYS TTLEDNFGII VDTKAYSEGY SLPISQADEM ERYVRENSNR
  121 DEEVNPNKWW ENFSEEVKKY YFVFISGSFK GKFEEQLRRL SMTTGVNGSA VNVVNLLLGA
  181 EKIRSGEMTI EELERAMFNN SEFILKY [symbol] DKKYSIGL AIGTNSVGWA VITDEYKVPS
  241 KKFKVLGNTD RHSIKKNLIG ALLFDSGETA EATRLKRTAR RRYTRRKNRI CYLQEIFSNE
  301 MAKVDDSFFH RLEESFLVEE DKKHERHPIF GNIVDEVAYH EKYPTIYHLR KKLVDSTDKA
  361 DLRLIYLALA HMIKFRGHFL IEGDLNPDNS DVDKLFIQLV QTYNQLFEEN PINASGVDAK
  421 AILSARLSKS RRLENLIAQL PGEKKNGLFG NLIALSLGLT PNFKSNFDLA EDAKLQLSKD
  481 TYDDDLDNLL AQIGDQYADL FLAAKNLSDA ILLSDILRVN TEITKAPLSA SMIKRYDEHH
  541 QDLTELKALV RQQLPEKYKE IFFDQSKNGY AGYIDGGASQ EEFYKFIKPI LEKMDGTEEL
  601 LVKLNREDLL RKQRTEDNGS IPHQIHLGEL HAILRRQEDF YPFLKDNREK IEKILTFRIP
  661 YYVGPLARGN SRFAWMTRKS EETITPWNFE EVVDKGASAQ SFIERMTNED KNLPNEKVLP
  721 KHSLLYEYFT VYNELTKVKY VTEGMRKPAF LSGEQKKAIV DLLFKTNRKV TVKQLKEDYF
  781 KKIECFDSVE ISGVEDRFNA SLGTYHDLLK IIKDKDFLDN EENEDILEDI VLTLTLFEDR
  841 EMIEERLKTY AHLFDDKVMK QLKRRRYTGW GRLSRKLING IRDKQSGKTI LDFLKSDGFA
  901 NRNFMQLIHD DSLTFKEDIQ KAQVSGQGDS LHEHIANLAG SPAIKKGILQ TVKVVDELVK
  961 VKGRHKPENI VIEMARENQT TQKGQKNSRE RMKRIEEGIK ELGSQILKEH PVENTQLQNE
1021 KLYLYYLQNG RDMYVDQELD INRLSDYDVD AIVPQSELKD DSIDNKVLTR SDKNRGKSDN
1081 VPSEEVVKKM KNYWRQLLNA KLITQRKEDN LTKAERGGLS ELDKAGFIKR QLVETRQITK
1141 HVAQILDSRM NTKYDENDKL IREVKVITLK SKLVSDFRKD FQFYKVREIN NYHHAHDAYL
1201 NAVVGTALIK KYPKLESEFV YGDYKVYDVR KMIAKSEQEI GKATAKYFFY SNIMNFFKTE
1261 ITLANGEIRK RPLIETNGET GEIVWDKGRD FATVRKVLSM PQVNIVKKTE VQTGGESKES
1321 ILPKRNSDKL IARKKDWDPK KYGGFDSPTV AYSVLVVAKV EKGKSKKLKS VKELLGITIM
1381 ERSSFEKNPI DFLEAKGYKE VKKDLIIKLP KYSLFELENG RKRMLASAGE LQKGNELALP
1441 SKYVNFLYLA SHYEKLKGSP EDNEQKQLEV EQHKHYLDEI IEQISEFSKR VILADANLDK
1501 VLSAYNKHRD KPIREQAENI IHLFTLTNLG APAAFKYFDT TIDRKRYTST KEVLDATLIH
1561 QSITGLYETR IDLSQLGGDG SPKKKRKV.
```

In certain embodiments, an exemplary dCas9-Clo051 fusion protein (embodiment 2) may comprise, consist essentially of or consist of, the nucleic acid sequence of (dCas9 sequence derived from Streptoccocus pyogenes):

```
(SEQ ID NO: 18060)
      1 atgcctaaga agaagcggaa ggtggaaggc atcaaaagca acatctccct cctgaaagac
    61 gaactccggg ggcagattag ccacattagt cacgaatacc tctccctcat cgacctggct
  121 ttcgatagca agcagaacag gctctttgag atgaaagtgc tggaactgct cgtcaatgag
  181 tacgggttca agggtcgaca cctcggcgga tctaggaaac cagacggcat cgtgtatagt
  241 accacactgg aagacaactt tgggatcatt gtggatacca aggcatactc tgagggttat
  301 agtctgccca tttcacaggc cgacgagatg gaacggtacg tgcgcgagaa ctcaaataga
  361 gatgaggaag tcaaccctaa caagtggtgg gagaacttct ctgaggaagt gaagaaatac
  421 tacttcgtct ttatcagcgg gtccttcaag ggtaaatttg aggaacagct caggagactg
  481 agcatgacta ccggcgtgaa tggcagcgcc gtcaacgtgg tcaatctgct cctgggcgct
  541 gaaaagattc ggagcggaga gatgaccatc gaagagctgg agagggcaat gtttaataat
  601 agcgagttta tcctgaaata cggtggcggt ggatccgata aaaagtattc tattggttta
  661 gccatcggca ctaattccgt tggatgggct gtcataaccg atgaatacaa agtaccttca
  721 aagaaattta aggtgttggg gaacacagac cgtcattcga ttaaaaagaa tcttatcggt
  781 gccctcctat tcgatagtgg cgaaacggca gaggcgactc gcctgaaacg aaccgctcgg
  841 agaaggtata cacgtcgcaa gaaccgaata tgttacttac aagaaatttt tagcaatgag
  901 atggccaaag ttgacgattc tttctttcac cgtttggaag agtccttcct tgtcgaagag
  961 gacaagaaac atgaacggca ccccatcttt ggaaacatag tagatgaggt ggcatatcat
1021 gaaaagtacc caacgattta tcacctcaga aaaaagctag ttgactcaac tgataaagcg
1081 gacctgaggt taatctactt ggctcttgcc catatgataa agttccgtgg gcactttctc
1141 attgagggtg atctaaatcc ggacaactcg gatgtcgaca aactgttcat ccagttagta
1201 caaacctata atcagttgtt tgaagagaac cctataaatg caagtggcgt ggatgcgaag
1261 gctattctta gcgcccgcct ctctaaatcc cgacggctag aaaacctgat cgcacaatta
1321 cccggagaga agaaaaatgg gttgttcggt aaccttatag cgctctcact aggcctgaca
1381 ccaaatttta agtcgaactt cgacttagct gaagatgcca aattgcagct tagtaaggac
1441 acgtacgatg acgatctcga caatctactg gcacaaattg gagatcagta tgcggactta
1501 tttttggctg ccaaaaacct tagcgatgca atcctcctat ctgacatact gagagttaat
1561 actgagatta ccaaggcgcc gttatccgct tcaatgatca aaaggtacga tgaacatcac
1621 caagacttga cacttctcaa ggccctagtc cgtcagcaac tgcctgagaa atataaggaa
1681 atattctttg atcagtcgaa aaacgggtac gcaggttata ttgacggcgg agcgagtcaa
1741 gaggaattct acaagtttat caaacccata ttagagaaga tggatgggac ggaagagttg
1801 cttgtaaaac tcaatcgcga agatctactg cgaaagcagc ggactttcga caacggtagc
1861 attccacatc aaatccactt aggcgaattg catgctatac ttagaaggca ggaggatttt
1921 tatccgttcc tcaaagacaa tcgtgaaaag attgagaaaa tcctaacctt tcgcatacct
1981 tactatgtgg gacccctggc ccgagggaac tctcggttcg catggatgac aagaaagtcc
2041 gaagaaacga ttactccatg gaattttgag gaagttgtcg ataaaggtgc gtcagctcaa
2101 tcgttcatcg agaggatgac caactttgac aagaatttac cgaacgaaaa agtattgcct
2161 aagcacagtt tactttacga gtatttcaca gtgtacaatg aactcacgaa agttaagtat
2221 gtcactgagg gcatgcgtaa acccgccttt ctaagcggag aacagaagaa agcaatagta
2281 gatctgttat tcaagaccaa ccgcaaagtg acagttaagc aattgaaaga ggactacttt
2341 aagaaaattg aatgcttcga ttctgtcgag atctccgggg tagaagatcg atttaatgcg
2401 tcacttggta cgtatcatga cctcctaaag ataattaaag ataaggactt cctggataac
2461 gaagagaatg aagatatctt agaagatata gtgttgactc ttaccctctt tgaagatcgg
2521 gaaatgattg aggaaagact aaaaacatac gctcacctgt tcgacgataa ggttatgaaa
2581 cagttaaaga ggcgtcgcta tacgggctgg ggacgattgt cgcggaaact tatcaacggg
2641 ataagagaca agcaaagtgg taaaactatt ctcgattttc taaagagcga cggcttcgcc
2701 aataggaact ttatgcagct gatccatgat gactctttaa ccttcaaaga ggatatacaa
2761 aaggcacagg tttccggaca aggggactca ttgcacgaac atattgcgaa tcttgctggt
2821 tcgccagcca tcaaaaaggg catactccag acagtcaaag tagtggatga gctagttaag
2881 gtcatgggac gtcacaaacc ggaaaacatt gtaatcgaga tggcacgcga aaatcaaacg
2941 actcagaagg ggcaaaaaaa cagtcgagag cggatgaaga gaatagaaga gggtattaaa
3001 gaactgggca gccagatctt aaaggagcat cctgtggaaa atacccaatt gcagaacgag
3061 aaactttacc tctattacct acaaaatgga agggacatgt atgttgatca ggaactggac
3121 ataaaccgtt tatctgatta cgacgtcgat gccattgtac cccaatcctt tttgaaggac
3181 gattcaatcg acaataaagt gcttacacgc tcggataaga accgagggaa aagtgacaat
3241 gttccaagcg aggaagtcgt aaagaaaatg aagaactatt ggcggcagct cctaaatgcg
3301 aaactgataa cgcaaagaaa gttcgataac ttaactaaag ctgagagggg tggcttgtct
3361 gaacttgaca aggccggatt tattaaacgt cagctcgtgg aaacccgcca aatcacaaag
3421 catgttgcac agatactaga ttcccgaatg aatacgaaat acgacgagaa cgataagctg
3481 attcgggaag tcaaagtaat cactttaaag tcaaaattgg tgtcggactt cagaaaggat
3541 tttcaattct ataaagttag ggagataaat aactaccacc atgcgcacga cgcttatctt
3601 aatgccgtcg tagggaccgc actcattaag aaatacccga agctagaaag tgagtttgtg
3661 tatggtgatt acaaagttta tgacgtccgt aagatgatcg cgaaaagcga acaggagata
3721 ggcaaggcta cagccaaata cttcttttat tctaacatta tgaatttctt taagacggaa
3781 atcactctgg caaacggaga gatacgcaaa cgacctttaa ttgaaaccaa tggggagaca
3841 ggtgaaatcg tatgggataa gggccgggac ttcgcgacgg tgagaaaagt tttgtccatg
3901 ccccaagtca acatagtaaa gaaaactgag gtgcagaccg gagggttttc aaaggaatcg
3961 attcttccaa aaaggaatag tgataagctc atcgctcgta aaaaggactg ggacccgaaa
4021 aagtacggtg gcttcgatag ccctacagtt gcctattctg tcctagtagt ggcaaaagtt
4081 gagaagggaa aatccaagaa actgaagtca gtcaaagaat tattggggat aacgattatg
4141 gagcgctcgt cttttgaaaa gaaccccatc gacttccttg aggcgaaagg ttacaaggaa
4201 gtaaaaaagg atctcataat taaactacca aagtatagtc tgtttgagtt agaaaatggc
4261 cgaaaacgga tgttggctag cgccggagag cttcaaaagg ggaacgaact cgcactaccg
4321 tctaaatacg tgaatttcct gtatttagcg tcccattacg agaagttgaa aggttcacct
4381 gaagataacg aacagaagca actttttgtt gagcagcaca aacattatct cgacgaaatc
4441 atagagcaaa tttcggaatt cagtaagaga gtcatcctag ctgatgccaa tctggacaaa
4501 gtattaagcg catacaacaa gcacagggat aaacccatac gtgagcaggc ggaaaatatt
4561 atccatttgt ttactcttac caacctcggc gctccagccg cattcaagta ttttgacaca
4621 acgatagatc gcaaacgata cacttctacc aaggaggtgc tagacgcgac actgattcac
4681 caatccatca cgggattata tgaaactcgg atagatttgt cacagcttgg gggtgacgga
4741 tcccccaaga agaagaggaa agtctga.
```

In certain embodiments, the nucleic acid sequence encoding a dCas9-Clo051 fusion protein (embodiment 2) of the disclosure may comprise a DNA. In certain embodiments, the nucleic acid sequence encoding a dCas9-Clo051 fusion protein (embodiment 2) of the disclosure may comprise an RNA.

### EXAMPLES

### Example 1: Construction and In Vitro Characterization of PSMA5 and PSMA8 CARTyrins

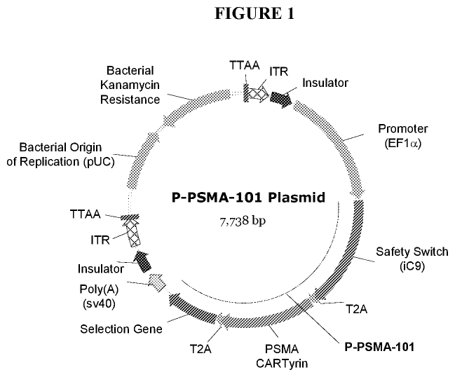

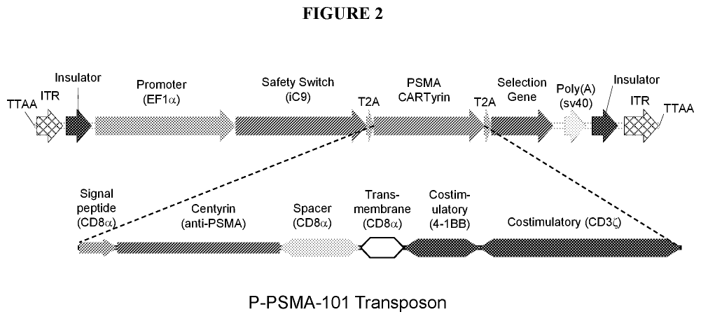

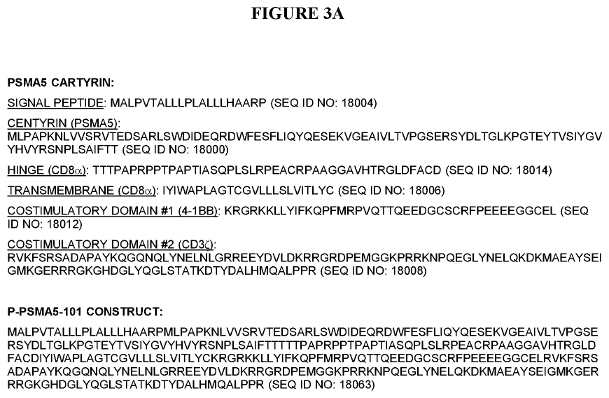

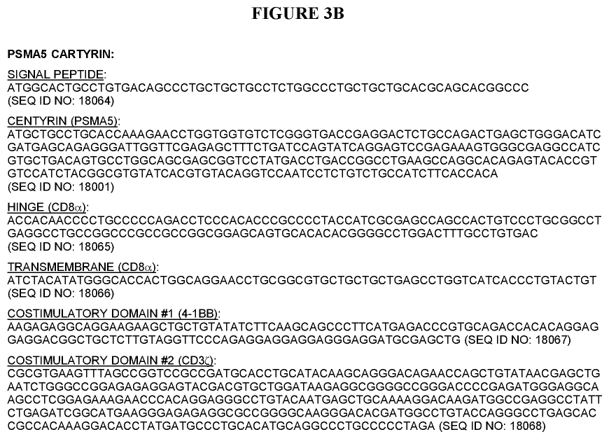

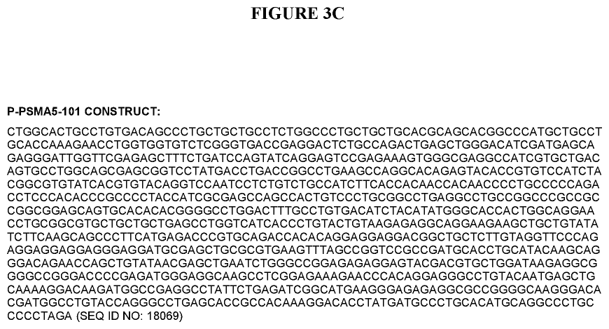

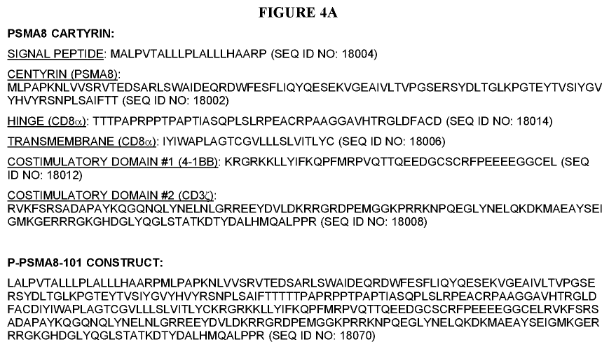

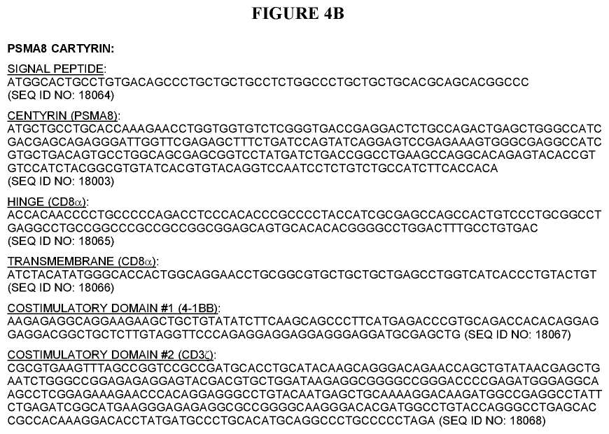

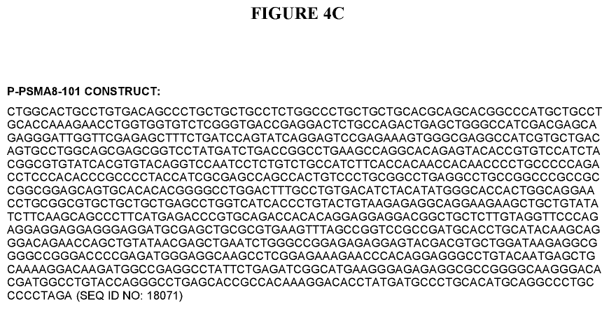

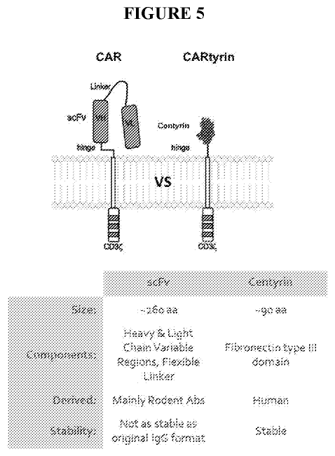

PSMA5 and PSMA8 CARTyrins were constructed as shown in FIGS. 1, 2, 3A-3C and 4A-4C. FIG. 5 depicts the structure of the anti-PSMA CARTyrins.

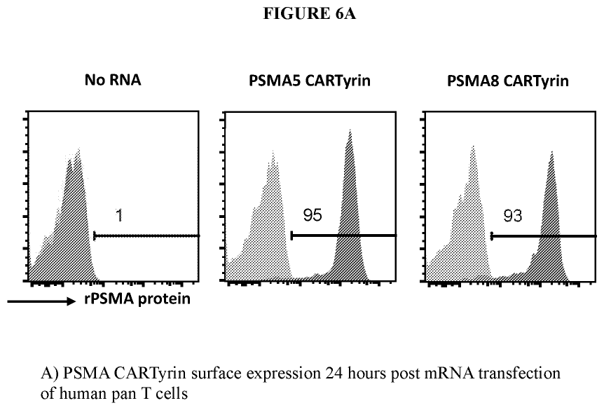

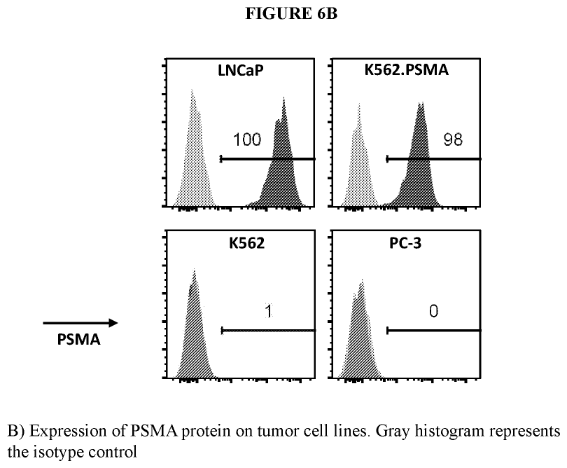

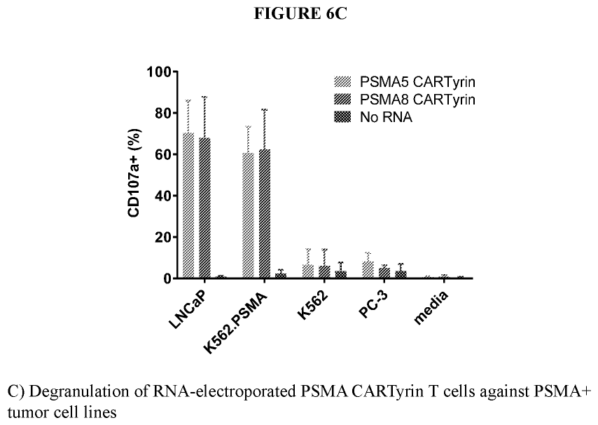

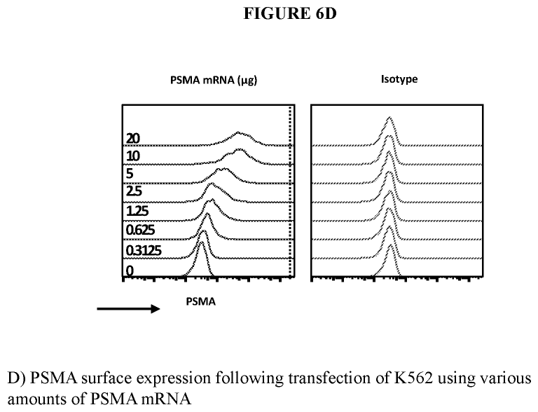

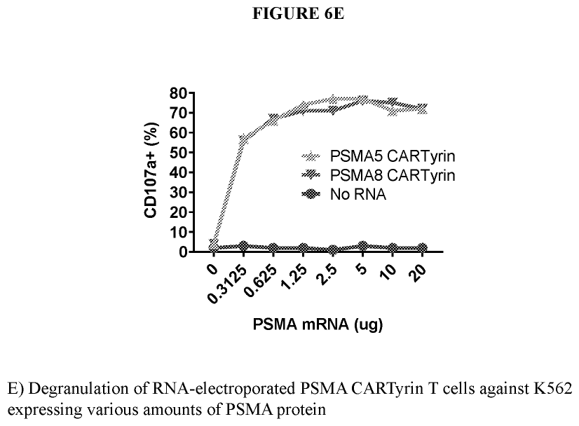

Surface expression of CARTyrins of the disclosure was evaluated by flow cytometry 24 hours following mRNA electroporation of a sequence encoding a PSMA5 or PSMA8 CARTyrin into human pan T cells (FIG. 6A). By flow cytometry, surface PSMA protein expression was detected on LNCaP tumor cell lines and K562 cell lines transfected with PSMA (FIG. 6B). Functionality of CARTyrin-expressing T cells was measured by degranulation against tumor lines. Degranulation of RNA-electroporated PSMA CARTyrin T cells against PSMA+ tumor cell lines was observed (FIG. 6C). PSMA surface protein expression was detected by flow cytometry following transfection of K562 cells lines using increasing amounts of PSMA mRNA (FIG. 6D). Degranulation of RNA-electroporated PSMA CARTyrin T cells against K562 expressing various amounts of PSMA protein was observed (FIG. 6E). Together, these data shows that PSMA5 and PSMA8 CARTyrins can be expressed on the surface of T cells and facilitate cytotoxic function against PSMA+ cell targets.

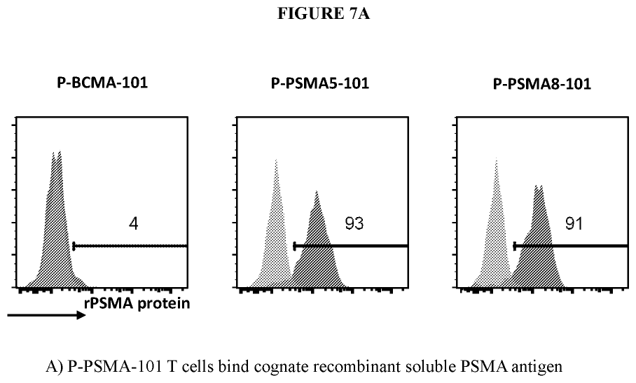

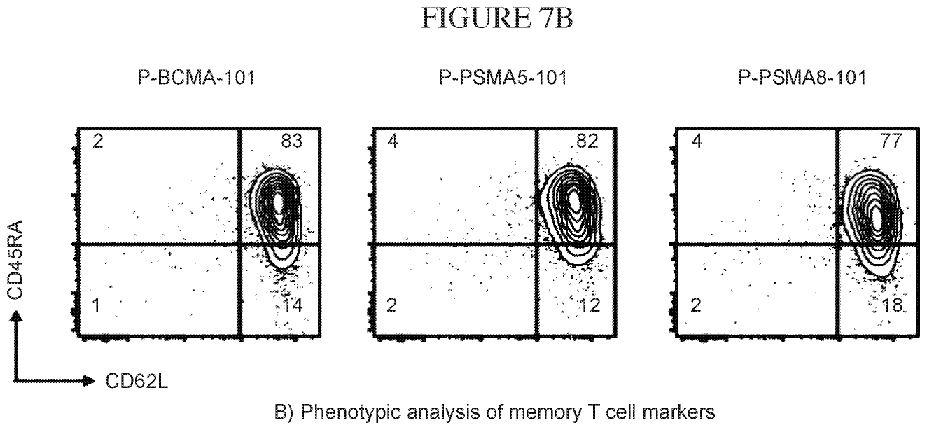

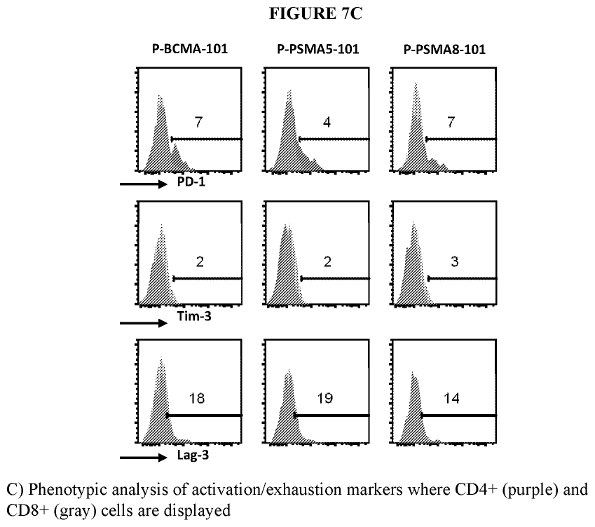

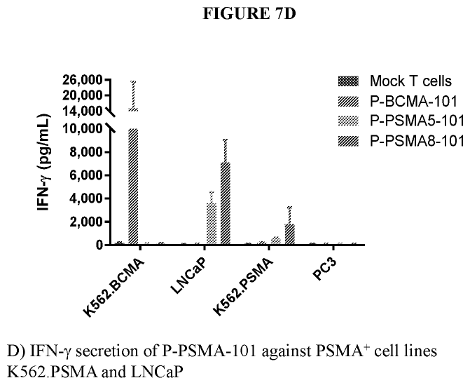

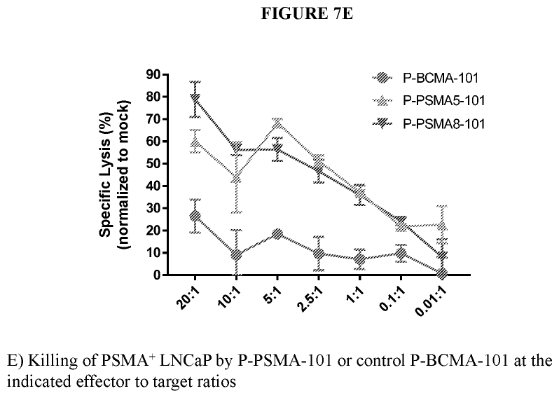

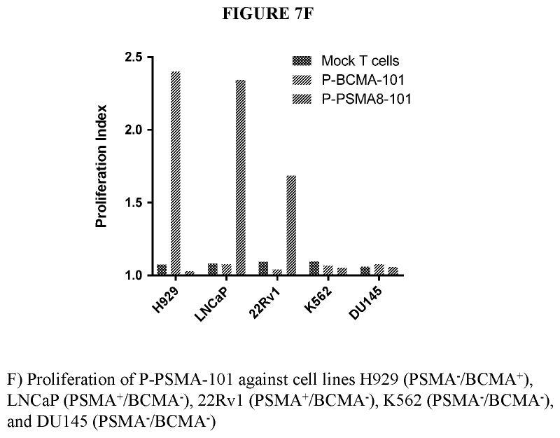

To support in vivo evaluation of PSMA CARTyrins, P-PSMA5-101 and P-PSMA8-101 were constructed using the piggyBac DNA modification system. PSMA CARTyrin was detected on the surface of primary human T cells from a representative donor that were transposed with either P-PSMA5-101 or P-PSMA8-101 plasmids (FIG. 7A). Flow cytometry analysis using surface expression markers showed that T cells expressing PSMA CARTyrin had markers of T stem cell memory phenotype but not activation and/or function a T cell exhaustion phenotype (FIG. 7B-7C). ELISA analysis showed that T cells expressing PSMA CARTyrins caused IFNγ secretion in cells expressing PSMA (LNCaP and K562.PSMA cells), demonstrating effector function of the T cells (FIG. 7D). T cells expressing PSMA CARTyrins showed strong cytotoxic function by a standard killing assay and a cell proliferation assay (FIG. 7E-7F). Together, these data show that surface expression of P-PSMA5-101 and P-PSMA8-101 PSMA CARTyrins on T cells demonstrated cytotoxic function and proliferative capacity in vitro against PSMA+ cell targets. Following this strong performance in vitro, the ability of the PSMA CARTyrins to function in vivo was evaluated.

### Example 2: In Vivo Characterization of PSMA5 and PSMA8 CARTyrins Using a Murine Xenograft Model

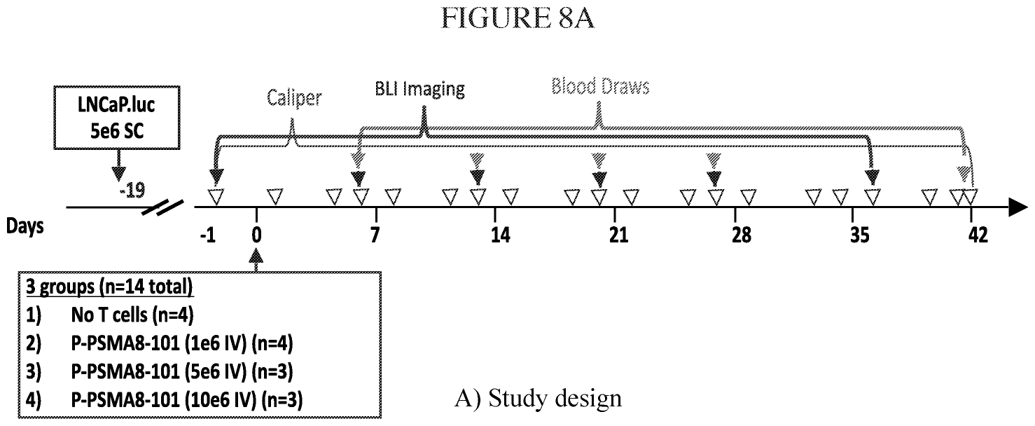

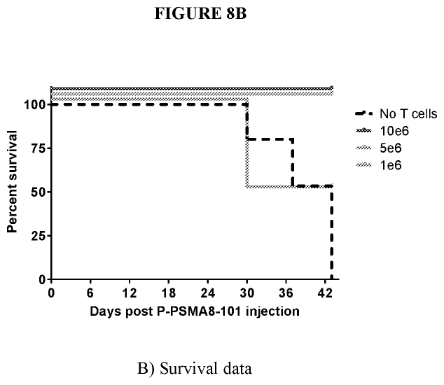

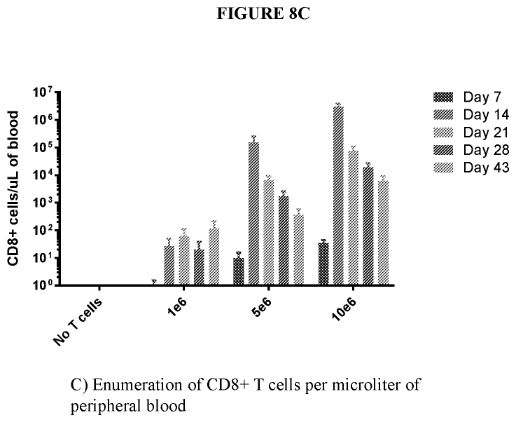

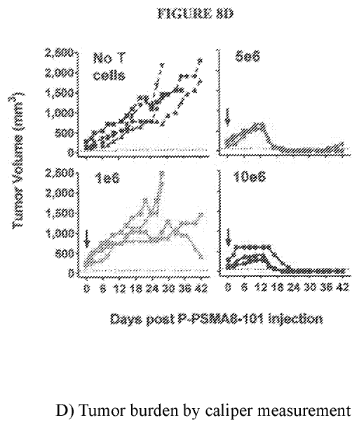

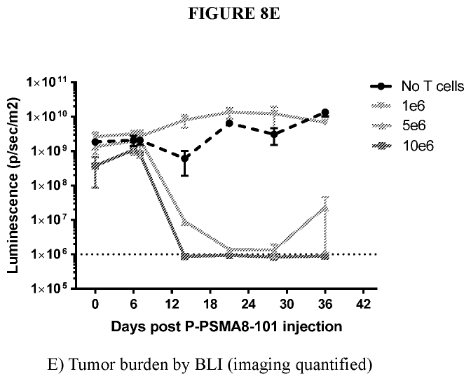

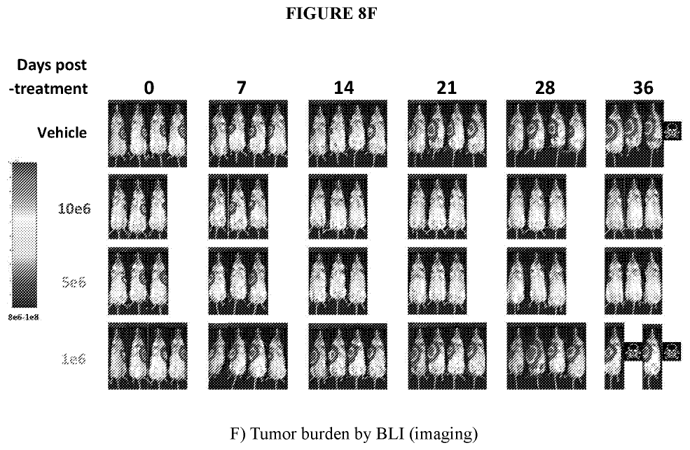

FIG. 8A depicts the treatment schedule for an in vivo study in mice using the P-PSMA8-101 CARTyrin. Anti-tumor activity was evaluated by survival (FIG. 8B), P-PSMA8-101 CD8+ T cell expansion and detection in the blood (FIG. 8C), tumor volume assessment by caliper measurement (FIG. 8D), and bioluminescence of LNCaP tumor (FIG. 8E-F). P-PSMA8-101 at ‘stress’ and standard doses demonstrated significantly enhanced anti-tumor efficacy and survival in comparison to ‘No T cells’ control mice against established SC LNCaP.luc solid tumors in NSG mice. Specifically, there was no survival in control animals, 50% survival in animal treated with an ‘ultra-low’ dose of P-PSMA8-101, and 100% survival in animals treated with either the ‘stress’ or standard dose of P-PSMA8-101. P-PSMA8-101 at the standard dose eliminated established LNCaP tumor in 100% of animals for the duration of the study (42 days post-treatment), while ⅔ of the ‘stress’ dosed animals remained tumor-free. In the peripheral blood, P-PSMA8-101 expanded and gave rise to differentiated effector PSMA8 CARTyrin+ T-cells that were concomitant with a decrease in tumor burden below detectable caliper and bioluminescent imaging limits. P-PSMA8-101 then contracted, yet persisted in the peripheral blood. Together this data demonstrates that animals expressing the PSMA8 CARTyrin demonstrate a reduce tumor burden compared to controls.

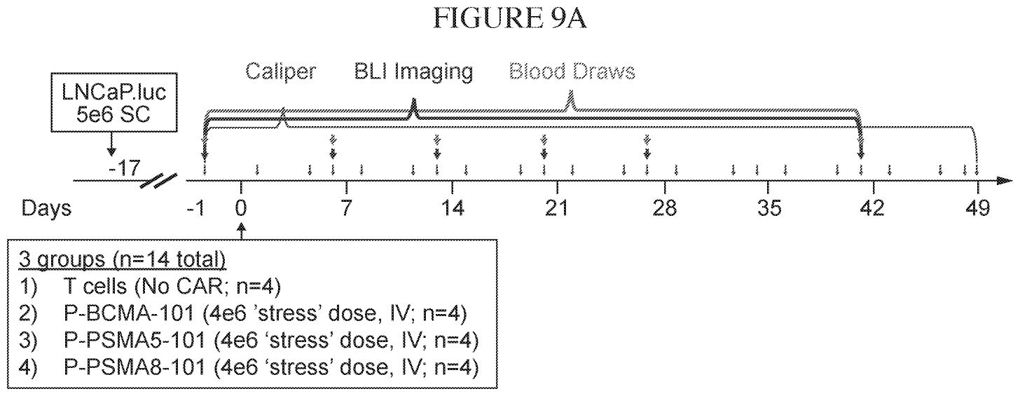

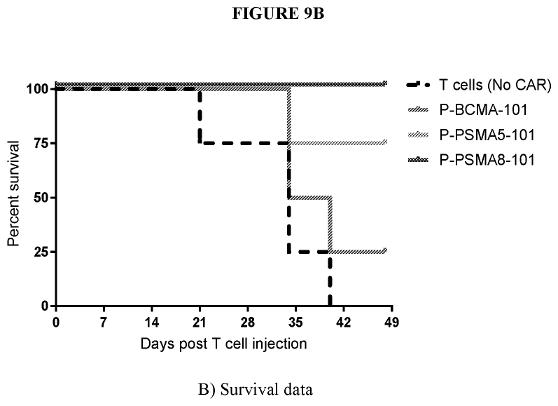

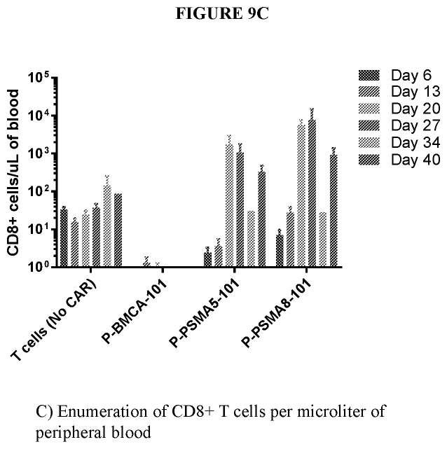

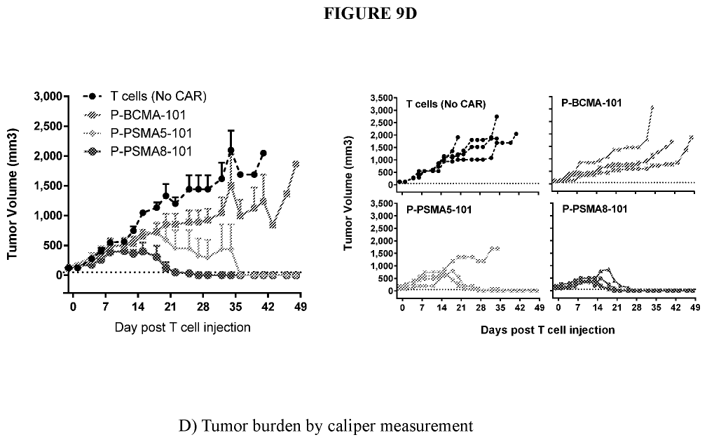

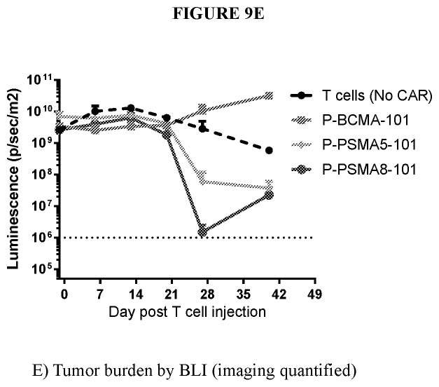

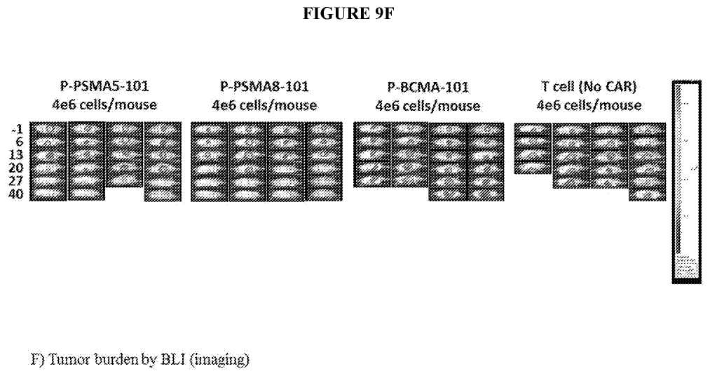

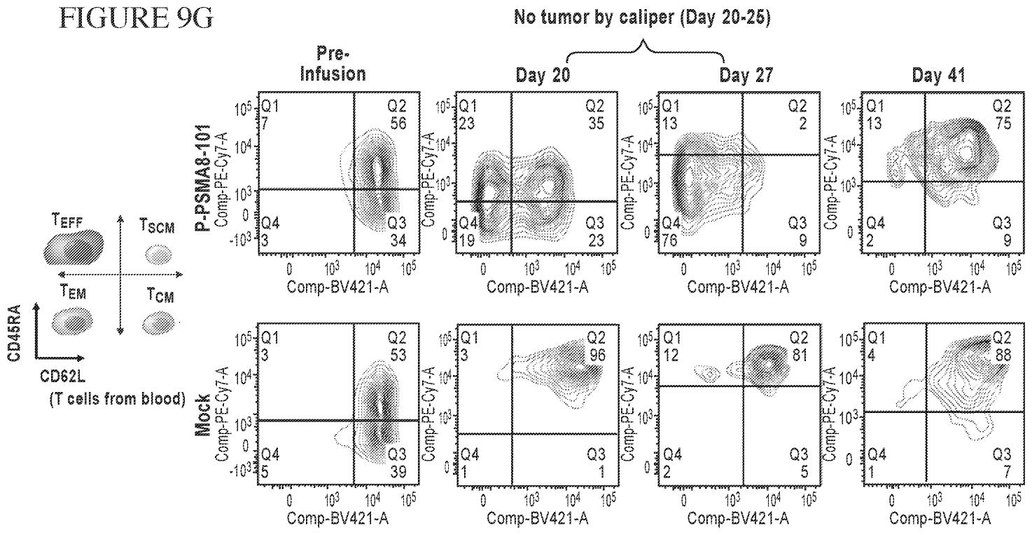

FIG. 9A depicts the treatment schedule for an in vivo study in mice using the P-PSMA5-101 and P-PSMA8-101 CARTyrins at a “stress” dose of (4×10\hat{ }6) using a murine xenograft model. Anti-tumor activity was evaluated by survival (FIG. 9B), CD8+ T cell expansion and detection in the blood (FIG. 9C), tumor volume assessment by caliper measurement (FIG. 9D), and bioluminescence of LNCaP tumor (FIG. 9E-F). P-PSMA5-101 and P-PSMA8-101 at a ‘stress’ dose demonstrated significantly enhanced anti-tumor efficacy and survival in comparison to the T cells (no CAR) control mice against established SC LNCaP.luc solid tumors in NSG mice. Specifically, there was no survival in T cells (no CAR) control animals, 25% survival in the P-BCMA-101 treated group, 75% survival in the P-PSMA5-101 treated group, and 100% survival in animals treated with a ‘stress’ dose of P-PSMA8-101. In the peripheral blood, P-PSMA5-101 and P-PSMA8-101 expanded and gave rise to differentiated effector CARTyrin+ T-cells that were concomitant with a decrease in tumor burden below detectable caliper and bioluminescent imaging limits. These cells then contracted, yet persisted in the peripheral blood.

### Example 3: Expression and Function of PiggyBac Integrated iC9 Safety Switch into Human Pan T-Cells

Human pan T-cells were nucleofected using an Amaxa 4D nucleofector with one of four piggyBac transposons. Modified T cells receiving the “mock” condition were nucleofected with an empty piggyBac transposon. Modified T cells received either a piggyBac transposon containing a therapeutic agent alone (a sequence encoding a CARTyrin) or a piggyBac transposon containing an integrated iC9 sequence and a therapeutic agent (a sequence encoding a CARTyrin).

FIG. 8 provides a schematic diagram of the iC9 safety switch, which contains a ligand binding region, a linker, and a truncated caspase 9 polypeptide. Specifically, the iC9 polypeptide contains a ligand binding region comprising a FK506 binding protein 12 (FKBP12) polypeptide including a substitution of valine (V) for phenylalanine (F) at position 36 (F36V). In certain embodiments, the FKBP12 polypeptide is encoded by an amino acid sequence comprising

(SEQ ID NO: 18026)
GVQVETISPGDGRTFPKRGQTCVVHYTGMLEDGKKVDSSRDRNKP
FKFMLGKQEVIRGWEEGVAQMSVGQRAKLTISPDYAYGATGHPGI
IPPHATLVFDVELLKLE.

In certain embodiments, the FKBP12 polypeptide is encoded by a nucleic acid sequence comprising

(SEQ ID NO: 18027)
GGGGTCCAGGTCGAGACTATTTCACCAGGGGATGGGCGAACATTTC
CAAAAAGGGGCCAGACTTGCGTCGTGCATTACACCGGGATGCTGGA
GGACGGGAAGAAAGTGGACAGCTCCAGGGATCGCAACAAGCCCTTC
AAGTTCATGCTGGGAAAGCAGGAAGTGATCCGAGGATGGGAGGAAG
GCGTGGCACAGATGTCAGTCGGCCAGCGGGCCAAACTGACCATTAG
CCCTGACTACGCTTATGGAGCAACAGGCCACCCAGGGATCATTCCC
CCTCATGCCACCCTGGTCTTCGATGTGGAACTGCTGAAGCTGGAG.

In certain embodiments, the induction agent specific for the ligand binding region may comprise a FK506 binding protein 12 (FKBP12) polypeptide having a substitution of valine (V) for phenylalanine (F) at position 36 (F36V) comprises AP20187 and/or AP1903, both synthetic drugs.

In certain embodiments of the inducible proapoptotic polypeptides, inducible caspase polypeptides or truncated caspase 9 polypeptides of the disclosure, the linker region is encoded by an amino acid comprising GGGGS (SEQ ID NO: 18028) or a nucleic acid sequence comprising GGAGGAGGAGGATCC (SEQ ID NO: 18029). In certain embodiments, the nucleic acid sequence encoding the linker does not comprise a restriction site.

In certain embodiments of the truncated caspase 9 polypeptides of the disclosure, the truncated caspase 9 polypeptide is encoded by an amino acid sequence that does not comprise an arginine (R) at position 87 of the sequence. Alternatively, or in addition, in certain embodiments of the inducible proapoptotic polypeptides, inducible caspase polypeptides or truncated caspase 9 polypeptides of the disclosure, the truncated caspase 9 polypeptide is encoded by an amino acid sequence that does not comprise an alanine (A) at position 282 the sequence. In certain embodiments of the inducible proapoptotic polypeptides, inducible caspase polypeptides or truncated caspase 9 polypeptides of the disclosure, the truncated caspase 9 polypeptide is encoded by an amino acid comprising

(SEQ ID NO: 18030)
GFGDVGALESLRGNADLAYILSMEPCGHCLIINNVNFCRESGLRTRT
GSNIDCEKLRRRFSSLHFMVEVKGDLTAKKMVLALLELAQQDHGALD
CCVVVILSHGCQASHLQFPGAVYGTDGCPVSVEKIVNIFNGTSCPSL
GGKPKLFFIQACGGEQKDHGFEVASTSPEDESPGSNPEPDATPFQEG
LRTFDQLDAISSLPTPSDIFVSYSTFPGFVSWRDPKSGSWYVETLDD
IFEQWAHSEDLQSLLLRVANAVSVKGIYKQMPGCFNFLRKKLFFKTS

or a nucleic acid sequence comprising

(SEQ ID NO: 18031)
GGATTTGGGGACGTGGGGGCCCTGGAGTCTCTGCGAGGAAATGCCGA
TCTGGCTTACATCCTGAGCATGGAACCCTGCGGCCACTGTCTGATCA
TTAACAATGTGAACTTCTGCAGAGAAAGCGGACTGCGAACACGGACT
GGCTCCAATATTGACTGTGAGAAGCTGCGGAGAAGGTTCTCTAGTCT
GCACTTTATGGTCGAAGTGAAAGGGGATCTGACCGCCAAGAAAATGG
TGCTGGCCCTGCTGGAGCTGGCTCAGCAGGACCATGGAGCTCTGGAT
TGCTGCGTGGTCGTGATCCTGTCCCACGGGTGCCAGGCTTCTCATCT
GCAGTTCCCCGGAGCAGTGTACGGAACAGACGGCTGTCCTGTCAGCG
TGGAGAAGATCGTCAACATCTTCAACGGCACTTCTTGCCCTAGTCTG
GGGGGAAAGCCAAAACTGTTCTTTATCCAGGCCTGTGGCGGGGAACA
GAAAGATCACGGCTTCGAGGTGGCCAGCACCAGCCCTGAGGACGAAT
CACCAGGGAGCAACCCTGAACCAGATGCAACTCCATTCCAGGAGGGA
CTGAGGACCTTTGACCAGCTGGATGCTATCTCAAGCCTGCCCACTCC
TAGTGACATTTTCGTGTCTTACAGTACCTTCCCAGGCTTTGTCTCAT
GGCGCGATCCCAAGTCAGGGAGCTGGTACGTGGAGACACTGGACGAC
ATCTTTGAACAGTGGGCCCATTCAGAGGACCTGCAGAGCCTGCTGCT
GCGAGTGGCAAACGCTGTCTCTGTGAAGGGCATCTACAAACAGATGC
CCGGGTGCTTCAATTTTCTGAGAAAGAAACTGTTCTTTAAGACTTCC.

In certain embodiments of the inducible proapoptotic polypeptides, wherein the polypeptide comprises a truncated caspase 9 polypeptide, the inducible proapoptotic polypeptide is encoded by an amino acid sequence comprising

(SEQ ID NO: 18032)
GVQVETISPGDGRTFPKRGQTCVVHYTGMLEDGKKVDSSRDRNKPFK
FMLGKQEVIRGWEEGVAQMSVGQRAKLTISPDYAYGATGHPGIIPPH
ATLVFDVELLKLEGGGGSGFGDVGALESLRGNADLAYILSMEPCGHC
LIINNVNFCRESGLRTRTGSNIDCEKLRRRFSSLHFMVEVKGDLTAK
KMVLALLELAQQDHGALDCCVVVILSHGCQASHLQFPGAVYGTDGCP
VSVEKIVNIFNGTSCPSLGGKPKLFFIQACGGEQKDHGFEVASTSPE
DESPGSNPEPDATPFQEGLRTFDQLDAISSLPTPSDIFVSYSTFPGF
VSWRDPKSGSWYVETLDDIFEQWAHSEDLQSLLLRVANAVSVKGIYK
QMPGCFNFLRKKLFFKTS

or the nucleic acid sequence comprising

(SEQ ID NO: 18033)
ggggtccaggtcgagactatttcaccaggggatgggcgaacatttccaaa
aaggggccagacttgcgtcgtgcattacaccgggatgctggaggacggga
agaaagtggacagctccagggatcgcaacaagcccttcaagttcatgctg
ggaaagcaggaagtgatccgaggatgggaggaaggcgtggcacagatgtc
agtcggccagcgggccaaactgaccattagccctgactacgcttatggag
caacaggccacccagggatcattccccctcatgccaccctggtcttcgat
gtggaactgctgaagctggagggaggaggaggatccggatttggggacgt
gggggccctggagtctctgcgaggaaatgccgatctggcttacatcctga
gcatggaaccctgcggccactgtctgatcattaacaatgtgaacttctgc
agagaaagcggactgcgaacacggactggctccaatattgactgtgagaa
gctgcggagaaggttctctagtctgcactttatggtcgaagtgaaagggg
atctgaccgccaagaaaatggtgctggccctgctggagctggctcagcag
gaccatggagctctggattgctgcgtggtcgtgatcctgtcccacgggtg
ccaggcttctcatctgcagttccccggagcagtgtacggaacagacggct
gtcctgtcagcgtggagaagatcgtcaacatcttcaacggcacttcttgc
cctagtctggggggaaagccaaaactgttctttatccaggcctgtggcgg
ggaacagaaagatcacggcttcgaggtggccagcaccagccctgaggacg
aatcaccagggagcaaccctgaaccagatgcaactccattccaggaggga
ctgaggacctttgaccagctggatgctatctcaagcctgcccactcctag
tgacattacgtgtcttacagtaccttcccaggctttgtctcatggcgcga
tcccaagtcagggagctggtacgtggagacactggacgacatctttgaac
agtgggcccattcagaggacctgcagagcctgctgctgcgagtggcaaac
gctgtctctgtgaagggcatctacaaacagatgcccgggtgcttcaattt
tctgagaaagaaactgttattaagacttcc.

To test the iC9 safety switch, each of the four modified T cells were incubated for 24 hours with 0, 0.1 nM, 1 nM, 10 nM, 100 nM or 1000 nM AP1903 (an induction agent for AP1903). Viability was assessed by flow cytometry using 7-aminoactinomycin D (7-AAD), a fluorescent intercalator, as a marker for cells undergoing apoptosis.

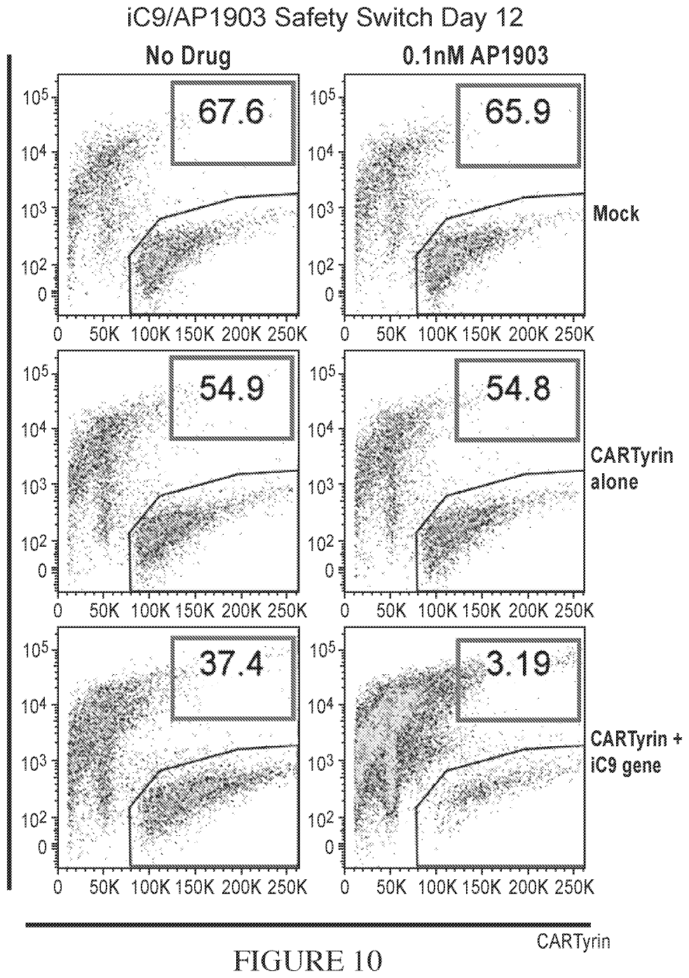

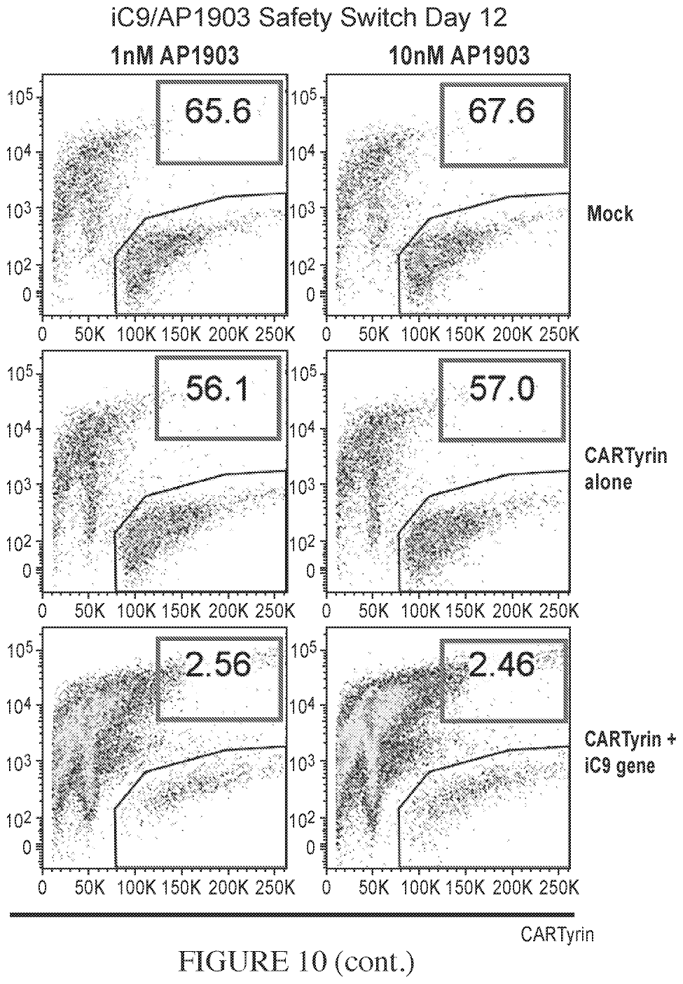

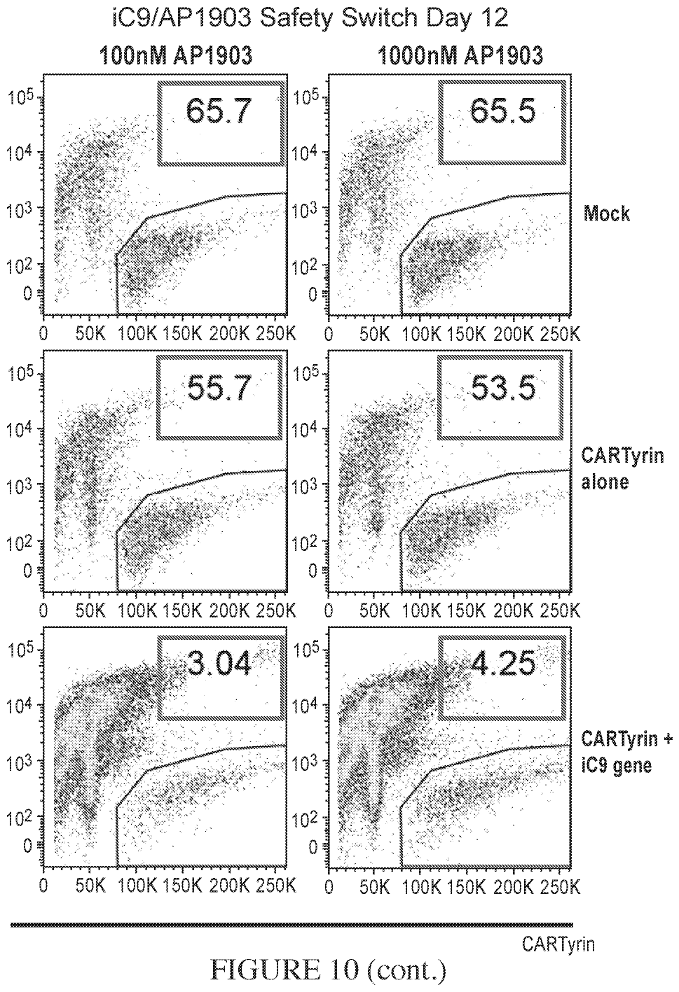

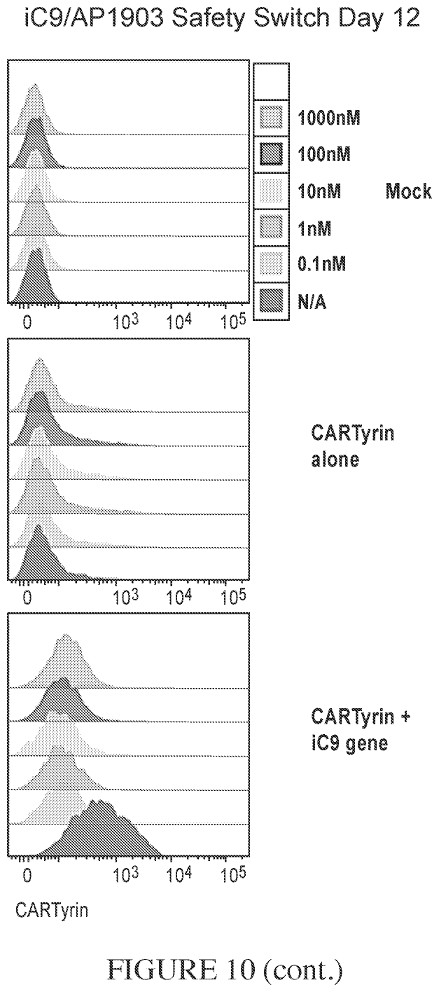

Cell viability was assessed at day 12 (see FIG. 9). The data demonstrate a shift of cell populations from the lower right to the upper left quadrants with increasing concentration of the induction agent in cells containing the iC9 construct; however, this effect is not observed in cells lacking the iC9 construct (those receiving only the CARTyrin), in which cells are evenly distributed among these two areas regardless of the concentration of the induction agent. Moreover, cell viability was assessed at day 19 (see FIG. 10). The data reveal the same trend as shown in FIG. 9 (day 12 post-nucleofection); however, the population shift to the upper left quadrant is more pronounced at this later time point (day 19 post-nucleofection).

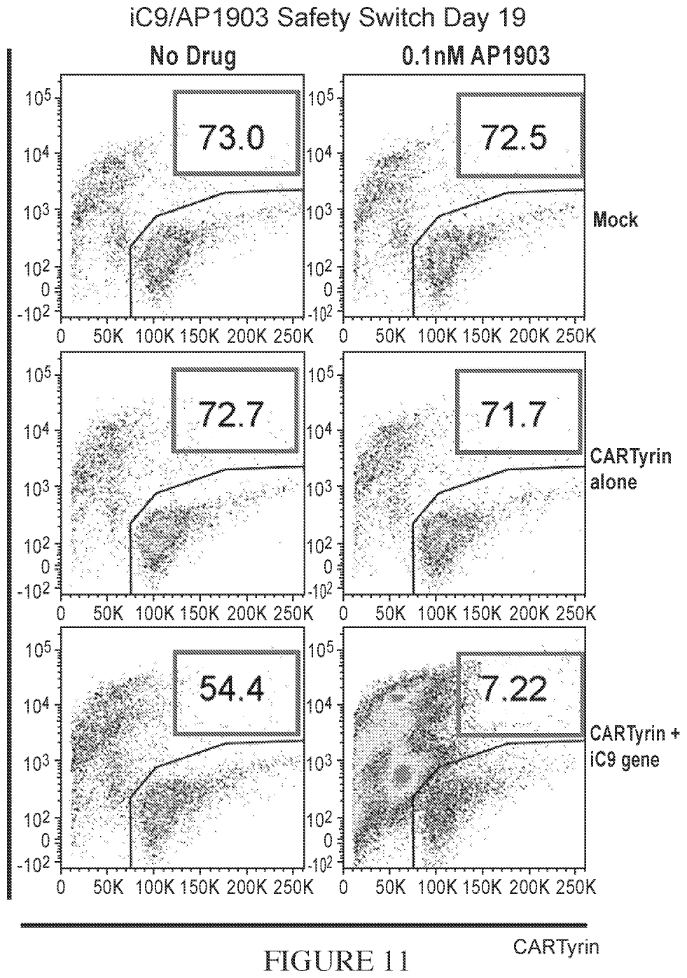

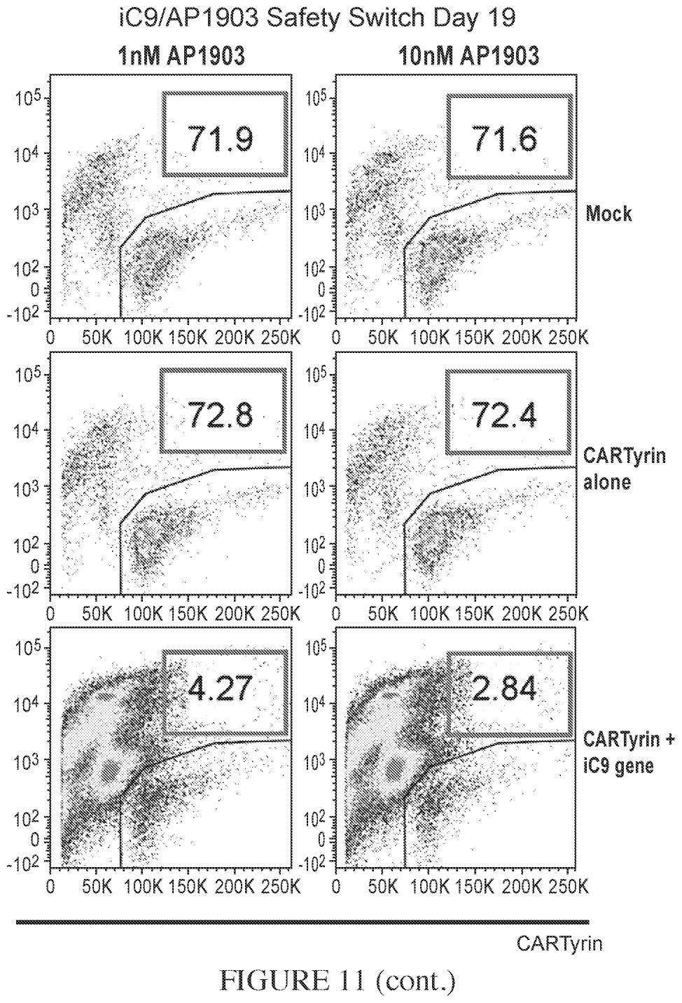

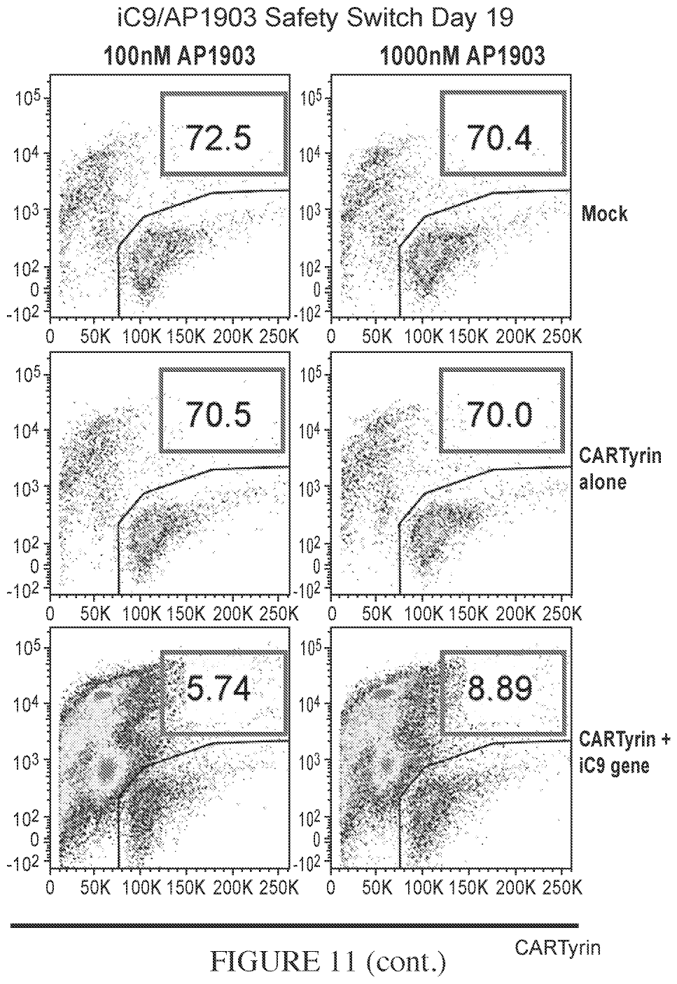

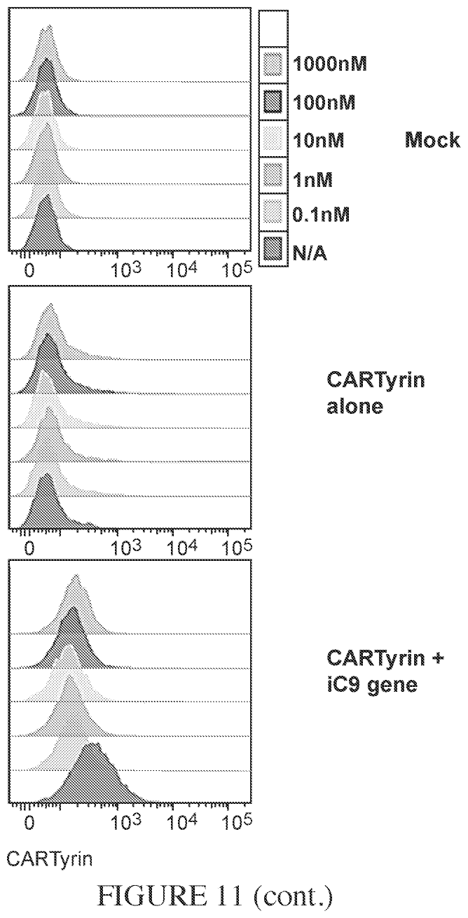

A quantification of the aggregated results was performed and is provided in FIG. 11, showing the significant impact of the iC9 safety switch on the percent cell viability as a function of the concentration of the induction agent (AP1903) of the iC9 switch for each modified cell type at either day 12 (FIG. 9 and left graph) or day 19 (FIG. 10 and right graph). The presence of the iC9 safety switch induces apoptosis in a significant majority of cells by day 12 and the effect is even more dramatic by day 19.

The results of this study show that the iC9 safety switch is extremely effective at eliminating active cells upon contact with an induction agent (e.g. AP1903) because AP1903 induces apoptosis at even the lowest concentrations of the study (0.1 nM). Furthermore, the iC9 safety switch may be functionally expressed as part of a tricistronic vector.

### Example 4: Knock Down Efficiency of Checkpoint Signaling Proteins on Armored T-Cells

To create armored T-cells which have enhanced therapeutic potential, genetic modifications may be made in order to render the T-cells less sensitive to immunologic and/or metabolic checkpoints. One mechanism to produce armored T-cells is to inhibit checkpoint signaling is to knockout various checkpoint receptors. The Cas-CLOVER™ platform was used to target and knockout the checkpoint receptors PD-1, TGFβR2, LAG-3, Tim-3, and CTLA-4 in resting (or quiescent) primary pan T cells. As measured by flow cytometry, gene editing resulted in 30-70% loss of protein expression at the cell surface (FIG. 13). These results show that Cas-CLOVER™ is able to efficiently target the knockout of these genes resulting in loss of target protein expression on the T-cell surface. Knockout efficiency can significantly be increased by further optimization of guide RNA pairs, or by using additional guide RNA pairs targeting the same gene and/or regulators or promoters of the target gene.

### Example 5: Strategies for the Expression of Null or Switch Intracellular Signaling Proteins on Armored T-Cells

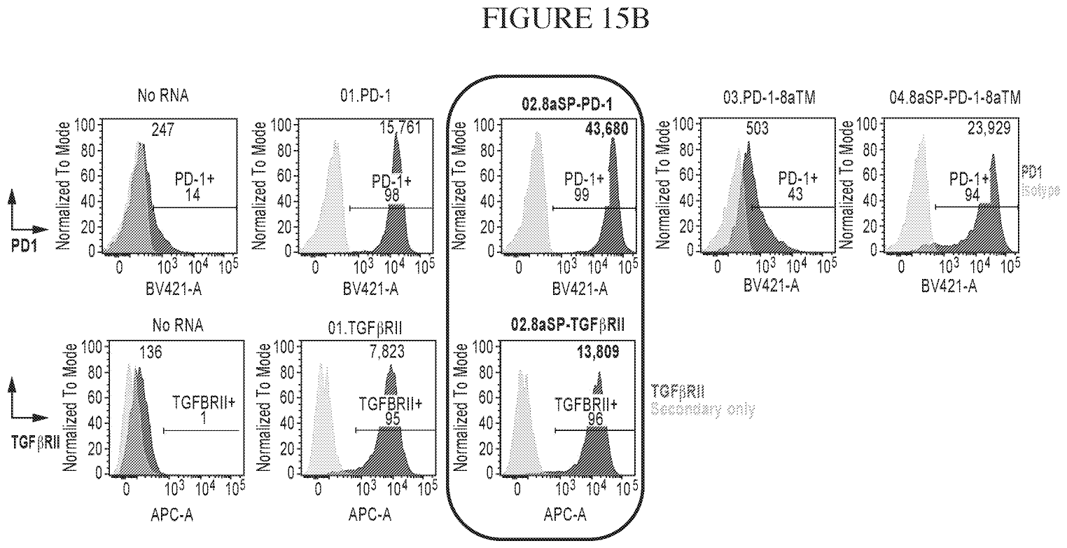

Another strategy to produce armored T-cells is to reduce or inhibit endogenous checkpoint signaling by expressing various modified/chimeric checkpoint receptors that have an altered or absent intracellular signaling domain. Checkpoint signals that could be targeted using this strategy include PD-1 or TGFβRII of T-cells, which bind to the PD-L1 ligand and TGFβ cytokine, respectively. FIG. 14 shows a schematic diagram of various strategies for producing decoy/null/dominant negative receptor (Null receptors) for two different inhibitory receptors (PD-1 (top panel) and TGFβRII (bottom panel)). To design Null receptors, the intracellular domain (ICD) of PD1 or TGFβRII can be mutated (mutated null) or deleted (truncated null). As a result, binding of the cognate ligand(s) of the null receptor does not result in delivery of the checkpoint signal to the T-cells. Furthermore, since the Null receptor competes with wildtype receptors for binding of the endogenous ligand(s), any binding by the Null receptor sequesters endogenous ligand(s) from binding the wildtype receptor. This results in dilution of the overall level of checkpoint signaling effectively delivered to the T-cell, thus, reducing or blocking checkpoint inhibition. FIG. 15 also shows switch receptor design strategies for the inhibitory receptors PD-1 (top panel) and TGFβRII (bottom panel). In switch receptors, wildtype ICD is replaced with the ICD from either an immuno-stimulatory molecule (Co-stimulatory switch) or a different inhibitory molecule (Inhibitory switch). Immuno-stimulatory molecules include but are not limited to CD3z, CD28, 4-1BB and the examples listed in Table 1. Inhibitory molecules include but are not limited to CTLA4, PD1, Lag3 and the examples listed in Tables 1 and 9. In the former case, binding of the endogenous ligand by the modified switch receptor results in the delivery of a positive signal to the T-cells, thereby helping to enhance stimulation of the T-cell, facilitating continuation of tumor targeting and killing. In the latter case, binding of the endogenous ligand by the modified switch receptor results in the delivery of a negative signal to the T-cells, thereby helping to reduce stimulation and activity of the T-cell.

### Example 6: Enhancing Surface Expression of PD1 and TGFβRII Null or Switch Intracellular Signaling Proteins on Armored T-Cells

To create armored T-cells, a number of truncated null receptors expressing alternative signal peptides (SP) and transmembrane domains (TM) were designed and tested for maximal expression on the surface of modified T-cells. FIG. 15A shows schematic diagrams of several null receptor constructs for PD-1 (top) and TGFβRII (bottom). Extracellular domains (ECD) of these proteins were modified such that the wildtype signal peptide (SP) and/or the transmembrane domains (TM) were replaced with that from the human T cell CD8α receptor (red arrows). Each of the six truncated null constructs shown in FIG. 15A were DNA synthesized and then subcloned into an mRNA IVT DNA vector (pRT). High quality mRNA was produced via IVT for each. Transfection of mRNA encoding each of the six molecules was performed using electroporation (EP) delivery into primary human T cells and FACS analysis was performed 24 hours post-EP to evaluate expression level of each construct on the cell surface (FIG. 15B). By flow cytometry, replacement of the WT SP with the alternative CD8a (02.8aSP-PD-1 and 02.8aSP-TGFβRII) resulted in the highest level of expression at the T cell surface. 02.8aSP-PD-1 Null receptor exhibited an MFI of 43,680, which is 177-fold higher than endogenous T cell PD-1 expression and 2.8-fold higher than the WT PD-1 Null receptor. 02.8aSP-TGFβRII Null receptor exhibited an MFI of 13,809, which is 102-fold higher than endogenous T cell TGFβRII expression and 1.8-fold higher than the WT TGFβRII Null receptor. These results show that replacement of wildtype SP with the alternative CD8a SP for both PD1 and TGFβRII inhibitory proteins leads to enhanced surface expression of the Null or Switch receptor. This in turn will maximize checkpoint inhibition or co-stimulation, respectively, upon binding of the endogenous ligand(s).

### Example 7: Design of NF-KB Inducible Vectors for Expression in Modified T-Cells

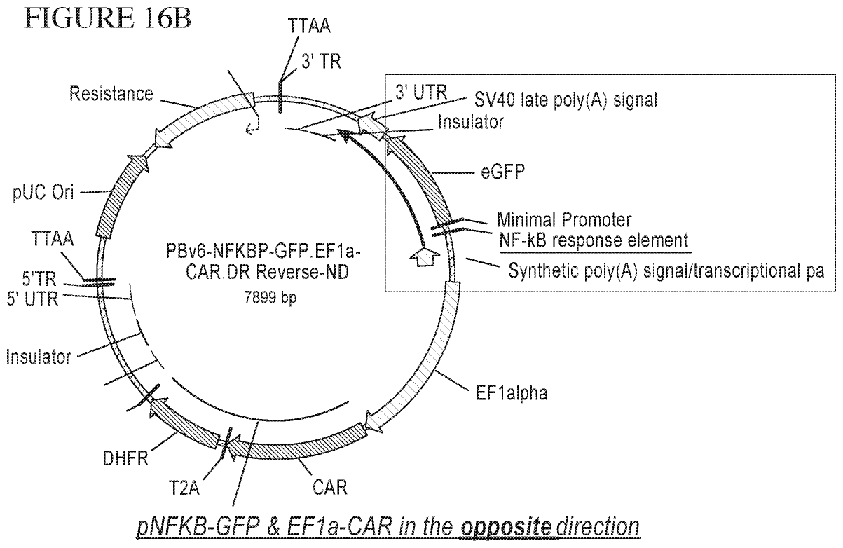

Two T cell activation NF-KB inducible vectors were developed (FIGS. 16A and 16B); one with the gene expression system (GES) in the forward orientation (A) and the other in the complementary direction (B), both preceding the constitutive EF1a promoter. These vectors also direct expression of a CAR molecule and a DHFR selection gene, separated by a T2A sequence. Both the conditional NF-KB inducible system and the EF1a directed genes are a part of a piggyBac transposon which can be permanently integrated into T cells using EP. Once integrated into the genome, the T cells constitutively express the CAR on the membrane surface and the DHFR within the cell, while expression of the NF-KB inducible gene, GFP, will be expressed to the highest level only upon T cell activation.

### Example 8: NF-KB Inducible Vectors for GFP Expression in Modified T-Cells

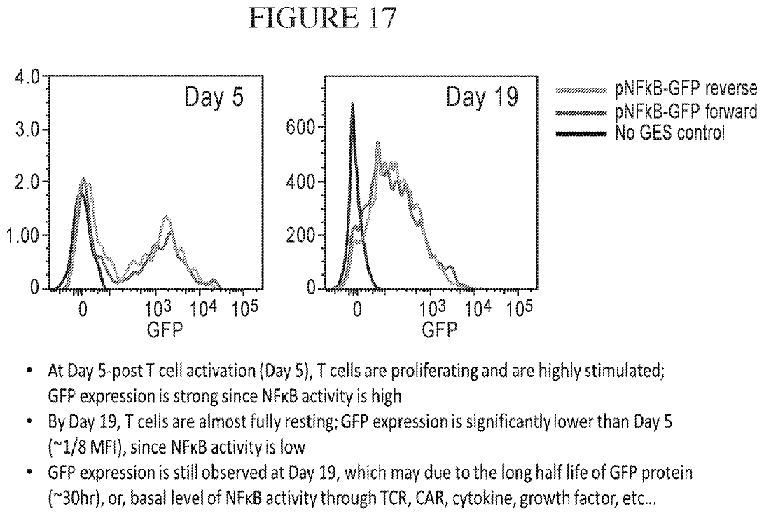

T cells were nucleofected with a piggyBac vector expressing an anti-BCMA CAR and a DHFR mutein gene under control of an EF1a promoter along with the absence (No gene expression system (GES) control) or presence of an NF-KB inducible expression system driving GFP expression in either the forward (pNFKB-GFP forward) or reverse orientation (pNFKB-GFP reverse). Cells were cultured in the presence of methotrexate selection until the cells were almost completely resting (Day 19) and GFP expression was assessed at Day 5 and Day 19. At Day 5, all T cells are proliferating and highly stimulated, with cells harboring the NF-KB inducible expression cassette producing high levels of GFP due to strong NFκB activity (see FIG. 17). The No GES control cells did not express detectable levels of GFP. By Day 19, the GES T cells were almost fully resting and GFP expression was significantly lower than Day 5 (˜⅛ MFI), since NFκB activity is lower. GFP expression is still observed at Day 19, which may due to the long half-life of GFP protein (˜30 hr), or, basal level of NFκB activity through, for example, a TCR, a CAR, a cytokine receptor, or a growth factor receptor signal.

### Example 9: NF-KB Inducible Vectors for Anti-BCMA CAR-Mediated GFP Expression in Modified T-Cells

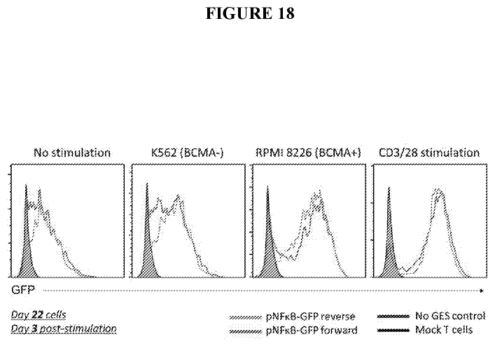

T cells were either unmodified (Mock T cells) or nucleofected with a piggyBac vector expressing an anti-BCMA CAR and a DHFR mutein gene under control of an EF1a promoter along with the absence (No GES control) or presence of an NF-KB inducible expression system driving GFP expression in either the forward (pNFKB-GFP forward) or reverse orientation (pNFKB-GFP reverse). All cells were cultured for 22 days, either with or without methotrexate selection (Mock T cells), until the cells were almost completely resting. Cells were then stimulated for 3 days in the absence (No stimulation) or presence of BCMA-(K562), BMCA+ (RPMI 8226), or positive control anti-CD3 anti-CD28 activation reagent (CD3/28 stimulation). GFP expression was undetectable under all conditions with the No GES control or Mock T cells. However, while pNFKB-GFP forward- and reverse-transposed cells exhibited little GFP expression over the No stimulation control when cultured with BCMA− K562 cells, they both demonstrated dramatic upregulation of gene expression either in the presence of BCMA+ tumor cells or under positive control conditions (FIG. 18). Little difference in GFP expression was observed between the pNFKB-GFP forward- and reverse-transposed cells that were cocultured with BCMA+ tumor cells.

### Example 10: Control of Anti-BCMA CAR-Mediated Expression in Modified T-Cells

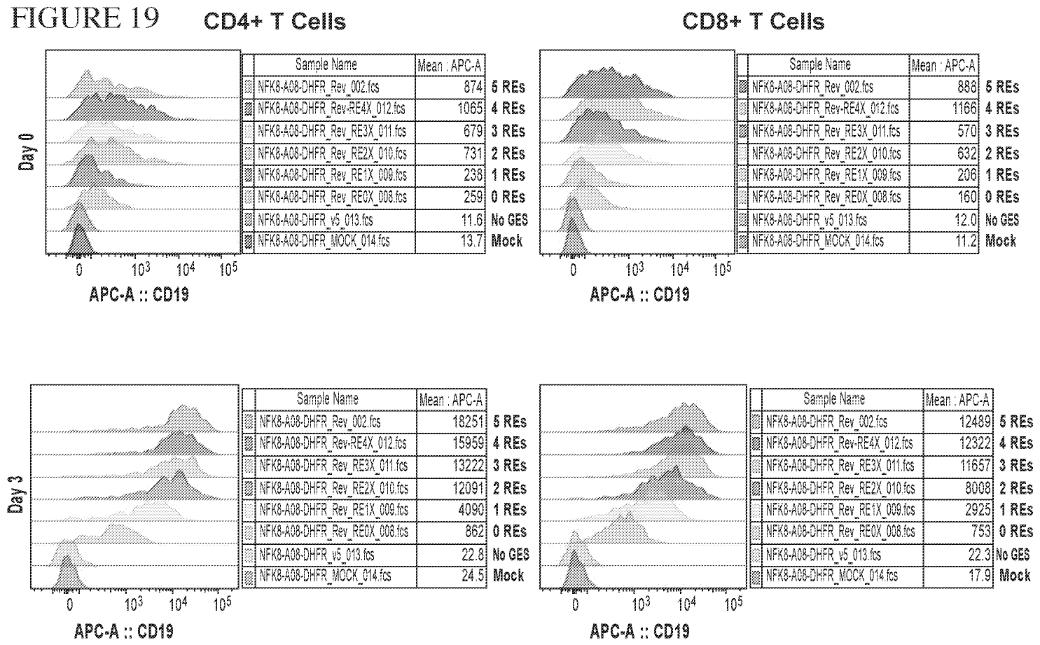

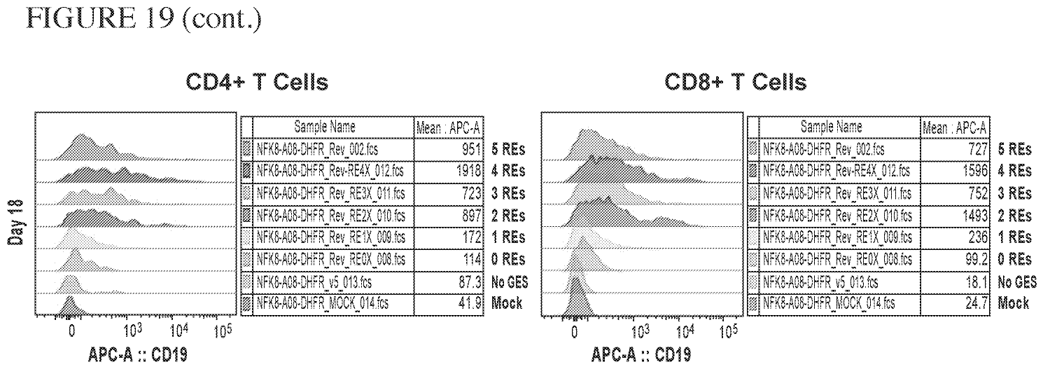

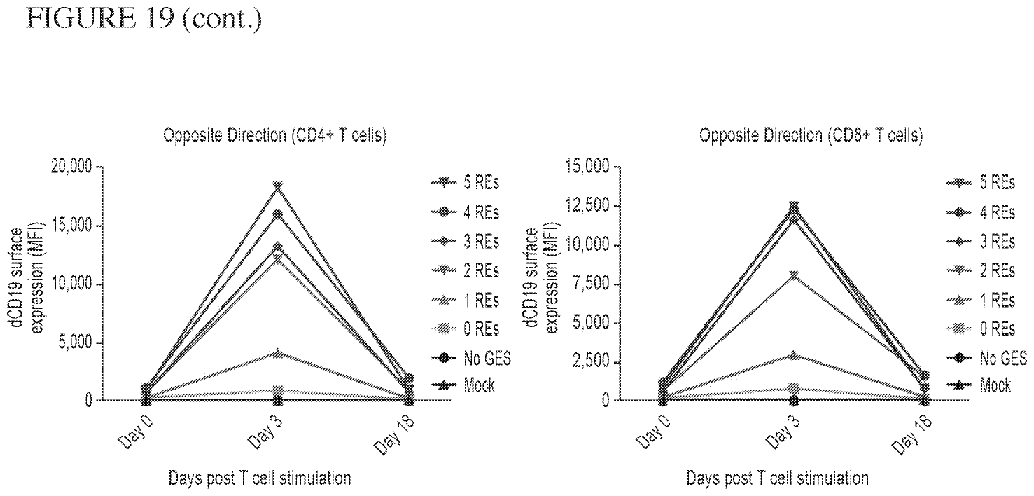

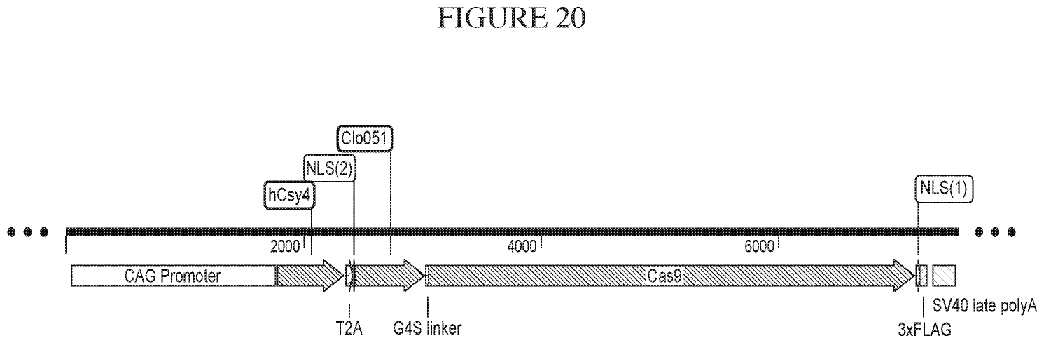

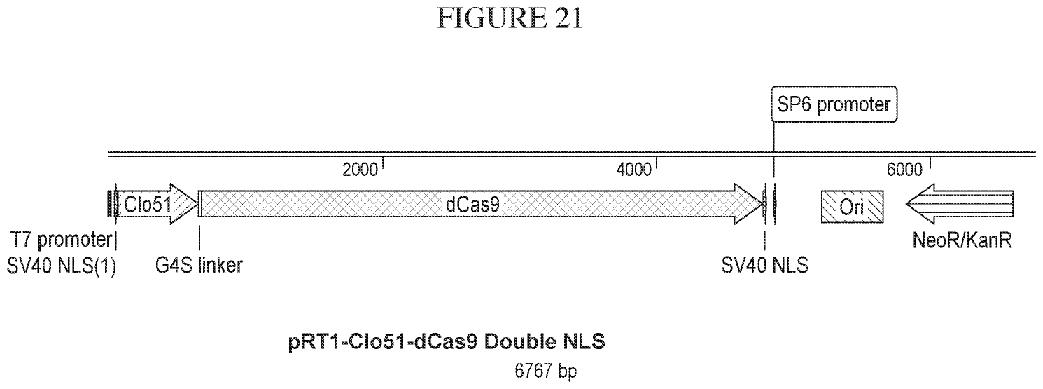

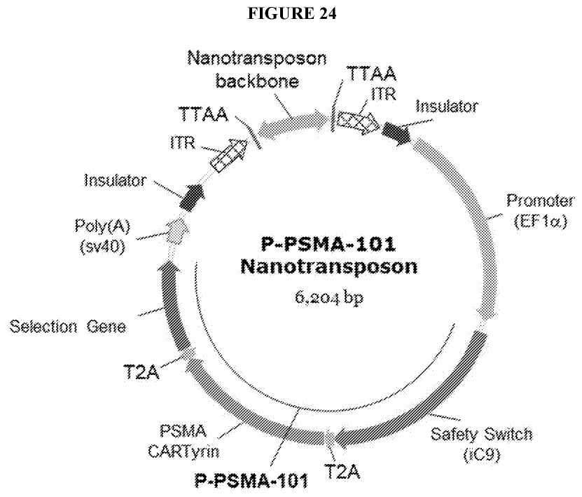

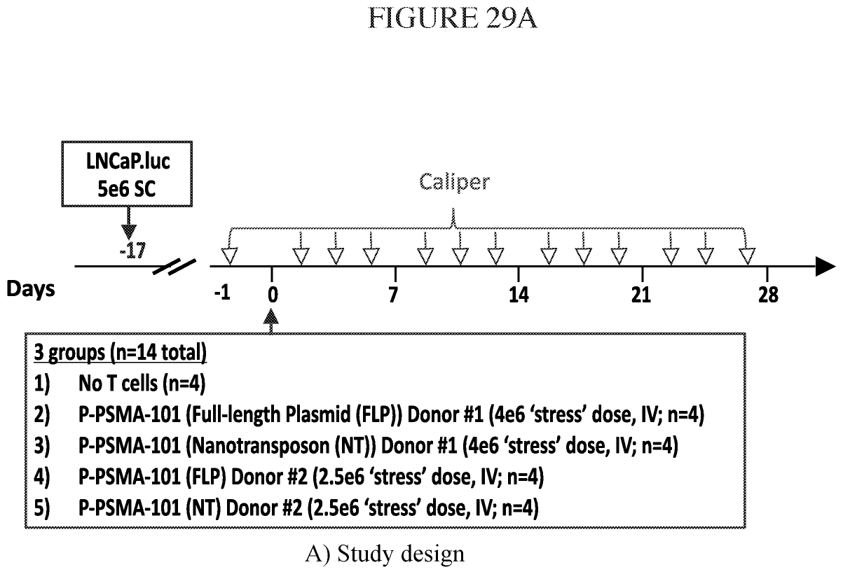

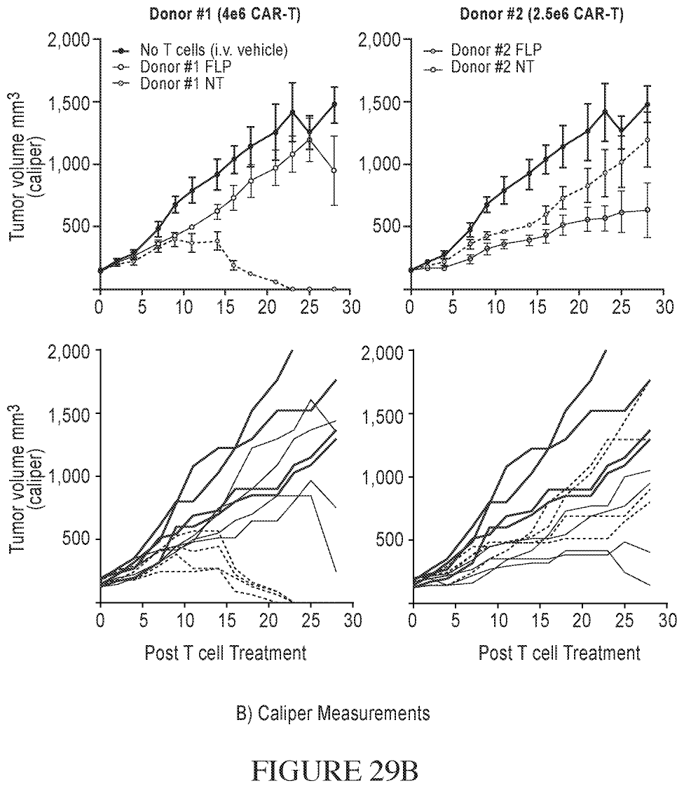

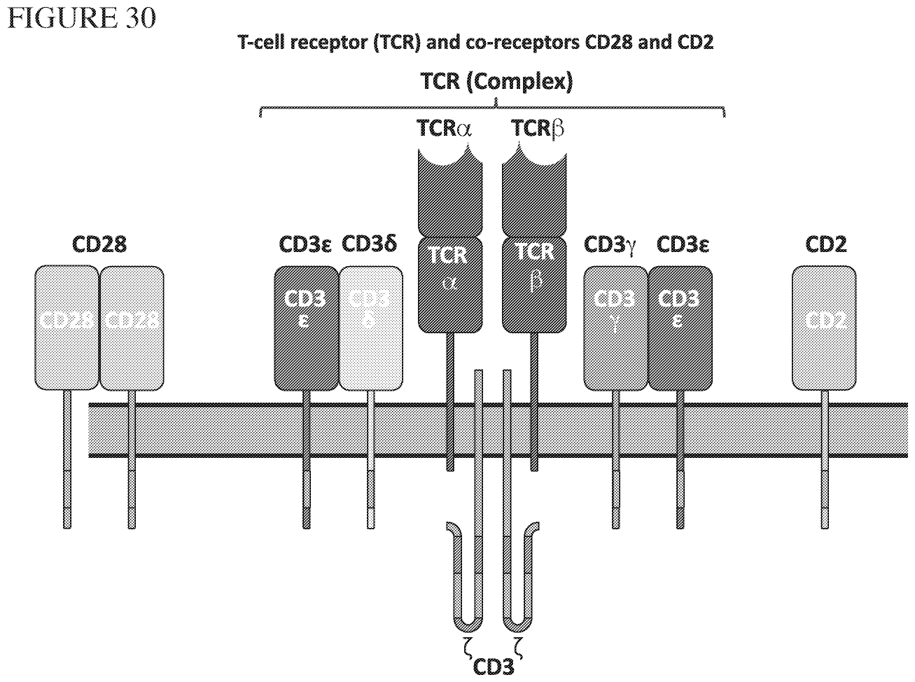

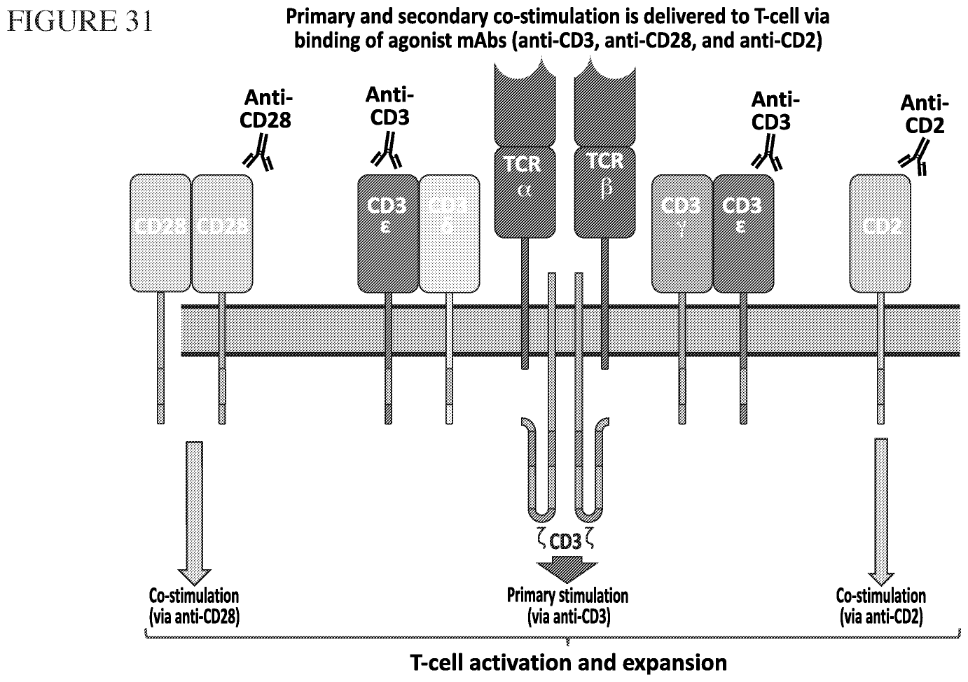

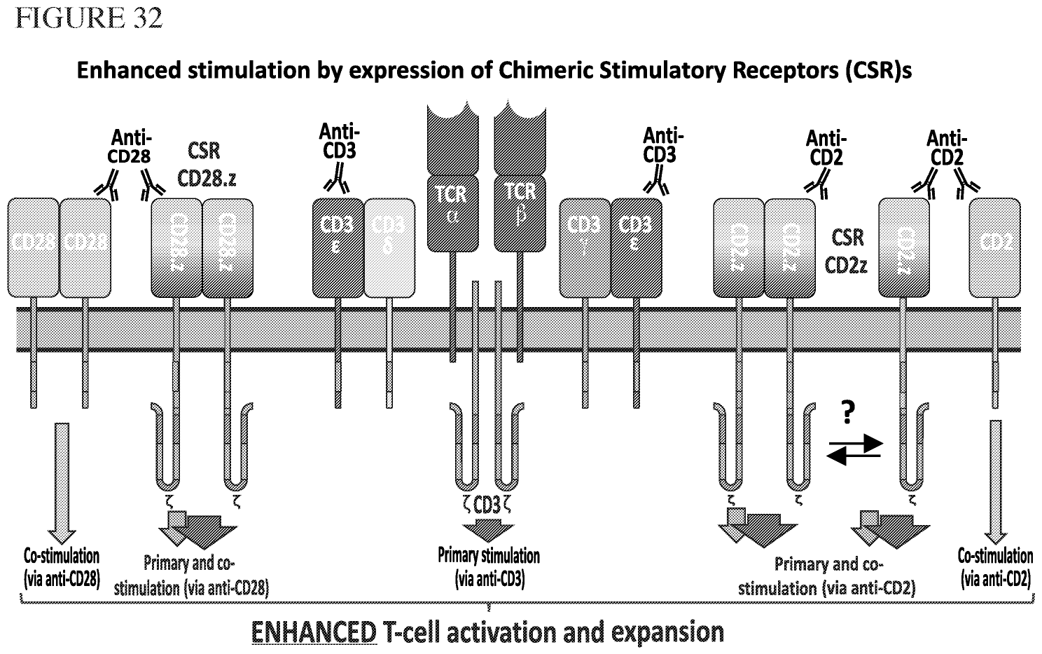

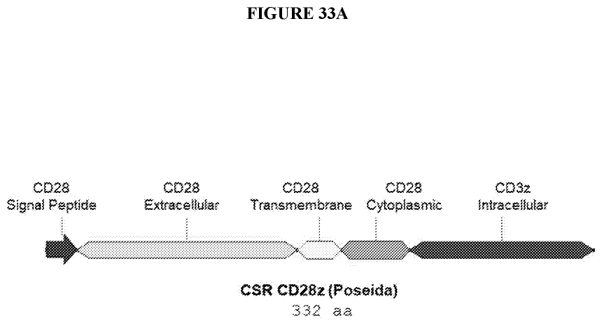

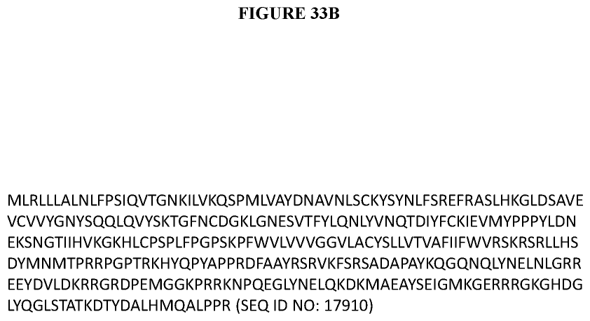

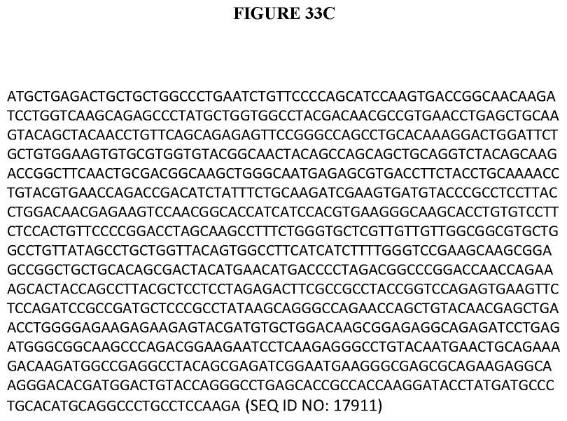

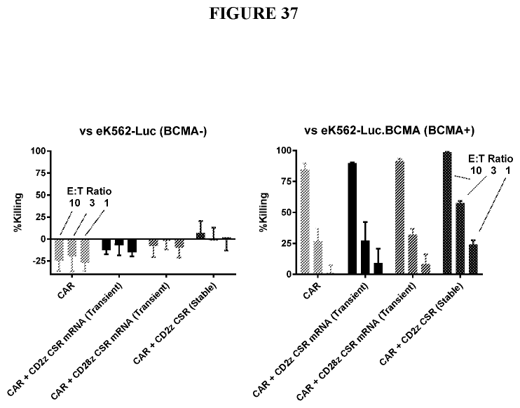

The expression level of inducible gene can be regulated by the number of response elements upstream or preceding the inducible promoter. T cells were nucleofected with a piggyBac vector encoding an anti-BCMA CARTyrin followed by a selection gene, both under control of a human EF1a promoter (FIG. 19). Further, vectors either additionally encoded the conditional NF-KB inducible gene expression system driving expression of a truncated CD19 protein (dCD19) and included a number of NFKB response elements (RE) varying from 0-5, no GES (No GES), or received an electroporation pulse but no piggyBac nucleic acid (Mock). Data are shown for only the GES in the reverse (opposite) direction/orientation. All cells were cultured for 18 days and included selection for piggyBac-modified T cells using methotrexate addition. Cells were then stimulated for 3 days using anti-CD3 anti-CD28 bead activation reagent and dCD19 surface expression was assessed by FACS at Days 0, 3 and 18, and data are shown as FACS histograms and MFI of target protein staining. Surface dCD19 expression was detected at low levels at Day 0 in all T cells transposed with vectors encoding the GES. At 3 days post-stimulation, dramatic upregulation of dCD19 expression was observed for all T cells expressing the GES, with a greater fold increase in surface expression in those with higher numbers of REs. Thus, surface dCD19 expression was directly proportional with the number of REs encoded in the GES. No dCD19 was detected on the surface of T cells that did not harbor the GES: No GES and Mock controls.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

1-273. (canceled)

274. A chimeric antigen receptor (CAR) comprising:
(a) an ectodomain comprising an antigen recognition region, wherein the antigen recognition region comprises at least one Centyrin that specifically binds prostate specific membrane antigen (PSMA);
(b) a transmembrane domain, and
(c) an endodomain comprising at least one costimulatory domain.

275. The CAR of claim 274, wherein the ectodomain of (a) further comprises a signal peptide.

276. The CAR of claim 274, wherein the ectodomain of (a) further comprises a hinge between the antigen recognition region and the transmembrane domain.

277. The CAR of claim 275, wherein the signal peptide comprises an amino acid sequence encoding a human CD2, CD3δ, CD3ε, CD3γ, CD3ζ, CD4, CD8α, CD19, CD28, 4-1BB or GM-CSFR signal peptide.

278. The CAR of claim 277, wherein the signal peptide comprises an amino acid sequence encoding a human CD8α signal peptide.

279. The CAR of claim 278, wherein the human CD8α signal peptide comprises an amino acid sequence of SEQ ID NO: 18004.

280. The CAR of claim 274, wherein the transmembrane domain comprises an amino acid sequence encoding a human CD2, CD3δ, CD3ε, CD3γ, CD3ζ, CD4, CD8α, CD19, CD28, 4-1BB or GM-CSFR transmembrane domain.

281. The CAR of claim 280, wherein the transmembrane domain comprises an amino acid sequence encoding a human CD8α transmembrane domain.

282. The CAR of claim 281, wherein the human CD8α transmembrane domain comprises an amino acid sequence of SEQ ID NO: 18006.

283. The CAR of claim 274, wherein the endodomain comprises a human CD3ζ endodomain.

284. The CAR of claim 274, wherein the at least one costimulatory domain comprises a human 4-1BB, CD28, CD40, ICOS, MyD88, OX-40 intracellular segment, or any combination thereof.

285. The CAR of claim 284, wherein the at least one costimulatory domain comprises a human 4-1BB costimulatory domain.

286. The CAR of claim 283, wherein the human CD3ζ endodomain domain comprises an amino acid sequence of SEQ ID NO: 18008.

287. The CAR of claim 285, wherein the human 4-1BB costimulatory domain comprises an amino acid sequence of SEQ ID NO: 18012.

288. The CAR of claim 276, wherein the hinge comprises an amino acid sequence derived from a human CD8α, IgG4, CD4 sequence or any combination thereof.

289. The CAR of claim 288, wherein the hinge comprises an amino acid sequence derived from a human CD8α.

290. The CAR of claim 289, wherein the sequence derived from a human CD8α comprises an amino acid sequence of SEQ ID NO: 18014.

291. The CAR of claim 274, wherein the at least one Centyrin that specifically binds PSMA comprises an amino acid sequence of SEQ ID NO: 18000.

292. The CAR of claim 274, wherein the at least one Centyrin that specifically binds PSMA is encoded by a nucleic acid sequence comprising SEQ ID NO: 18001.

293. The CAR of claim 274, wherein the at least one Centyrin that specifically binds PSMA comprises an amino acid sequence of SEQ ID NO: 18002.

294. The CAR of claim 274, wherein the at least one Centyrin that specifically binds PSMA is encoded by a nucleic acid sequence comprising SEQ ID NO: 18003.

295. A composition comprising the CAR of claim 274 and at least one pharmaceutically acceptable carrier.

296. A transposon comprising a nucleic acid sequence encoding the CAR of claim 274.

297. A composition comprising the transposon of claim 296.

298. A vector comprising a nucleic acid sequence encoding the CAR of claim 274.

299. A composition comprising the vector of claim 298.

300. A cell comprising the CAR of claim 274.

301. A cell comprising the transposon of claim 296.

302. A cell comprising the vector of claim 298.

303. The cell of claim 300, wherein the cell is an immune cell.

304. The cell of claim 303, wherein the immune cell is a T-cell, a Natural Killer (NK) cell, a Natural Killer (NK)-like cell, a Cytokine Induced Killer (CIK) cell, a hematopoietic progenitor cell, a peripheral blood (PB) derived T cell or an umbilical cord blood (UCB) derived T-cell.

305. The cell of claim 300, wherein the cell is autologous.

306. A composition comprising a population of cells, wherein the plurality of cells of the population comprises the cell of claim 300.

307. A method of producing a population of modified T-cells comprising:
(a) introducing into a plurality of T-cells a composition comprising a nucleic acid sequence encoding the CAR of claim 1 thereby generating a modified T-cell population;
(b) culturing the modified T-cell population under conditions suitable for integration of the nucleic acid sequence encoding the CAR;
(c) expanding and/or selecting at least one cell from the modified T-cell population that expresses the CAR on the cell surface.

308. The CAR of claim 274, comprising the amino acid sequence of SEQ ID NO: 18063.

309. The CAR of claim 274, comprising the amino acid sequence of SEQ ID NO: 18070.

310. A chimeric antigen receptor (CAR) comprising:
(a) an ectodomain comprising a human CD8α signal peptide and an antigen recognition region, wherein the antigen recognition region comprises at least one Centyrin that specifically binds PSMA;
(b) a hinge domain comprising a human CD8α hinge domain;
(c) a transmembrane domain comprising a human CD8α transmembrane domain; and
(d) an endodomain comprising a human 4-1BB costimulatory domain and a human CD3ζ endodomain.

311. The CAR of claim 310, wherein the at least one Centyrin that specifically binds PSMA comprises the amino acid sequence of SEQ ID NO: 18000, wherein the human CD8α signal peptide comprises the amino acid sequence of SEQ ID NO: 18004, wherein the human CD8α hinge domain comprises the amino acid sequence of SEQ ID NO: 18014, wherein the human CD8α transmembrane domain comprises the amino acid sequence of SEQ ID NO: 18006, wherein the human 4-1BB costimulatory domain comprises the amino acid sequence of SEQ ID NO: 18012, and wherein the human CD3ζ endodomain comprises the amino acid sequence of SEQ ID NO: 18008.

312. The CAR of claim 310, wherein the at least one Centyrin that specifically binds PSMA comprises the amino acid sequence of SEQ ID NO: 18002, wherein the human CD8α signal peptide comprises the amino acid sequence of SEQ ID NO: 18004, wherein the human CD8α hinge domain comprises the amino acid sequence of SEQ ID NO: 18014, wherein the human CD8α transmembrane domain comprises the amino acid sequence of SEQ ID NO: 18006, wherein the human 4-1BB costimulatory domain comprises the amino acid sequence of SEQ ID NO: 18012, and wherein the human CD3ζ endodomain comprises the amino acid sequence of SEQ ID NO: 18008.
